(19) United States
(12) Patent Application Publication
(10) Pub. No.: US 2022/0370431 A1
(43) Pub. Date: Nov. 24, 2022

(54) C-MYC MRNA TRANSLATION MODULATORS AND USES THEREOF IN THE TREATMENT OF CANCER

(71) Applicant: Anima Biotech Inc., Bernardsville, NJ (US)

(72) Inventors: David William SHEPPARD, Ramat Gan (IL); Jason Paul TIERNEY, Ramat Gan (IL); Aviad MANDABI, Ramat Gan (IL); Iris ALROY, Ramat Gan (IL); Rina WASSERMANN, Ramat Gan (IL); Yaode WANG, Beijing (CN); Haitang LI, Beijing (IL); Yoni SHEINBERGER, Ramat Gan (IL)

(73) Assignee: Anima Biotech Inc., Bernardsville, NJ (US)

(21) Appl. No.: 17/856,998

(22) Filed: Jul. 3, 2022

Related U.S. Application Data
(63) Continuation-in-part of application No. PCT/US2002/011203, filed on Jan. 5, 2022.

(30) Foreign Application Priority Data
Jan. 5, 2021 (IL) 279972

(51) Int. Cl. A61K 31/454; A61P 35/00; C07D 513/04; A61K 31/437; C07D 513/14; A61K 31/429; A61K 31/5377; A61K 31/541; A61K 31/55; A61K 31/496; C07D 519/00

(52) U.S. Cl. CPC A61K 31/454; A61P 35/00; C07D 513/04; A61K 31/437; C07D 513/14; A61K 31/429; A61K 31/5377; A61K 31/541; A61K 31/55; A61K 31/496; C07D 519/00

(57) ABSTRACT

The present invention relates to novel c-MYC mRNA translation modulators, composition and methods of preparation thereof, and uses thereof in the treatment of cancer.

### CROSS-REFERENCE TO RELATED APPLICATIONS

This Application is a Continuation-In-Part of PCT Application Number PCT/US2022/011203, filed Jan. 5, 2022; which claims priority of Israeli Patent Application serial number 279972, filed Jan. 5, 2021; both of which are herein incorporated by reference in their entirely.

### FIELD OF THE INVENTION

The present invention relates to novel c-MYC mRNA translation modulators, composition and methods of preparation thereof, and uses thereof in the treatment of cancer.

### BACKGROUND OF THE INVENTION

Cancer is the second most common cause of death in the United States, exceeded only by heart disease. In the United States, cancer accounts for 1 of every 4 deaths. The 5-year relative survival rate for all cancer patients diagnosed in 1996-2003 is 66%, up from 50% in 1975-1977 (Cancer Facts & Figures American Cancer Society: Atlanta, Ga. (2008)). The rate of new cancer cases decreased by an average 0.6% per year among men between 2000 and 2009 and stayed the same for women. From 2000 through 2009, death rates from all cancers combined decreased on average 1.8% per year among men and 1.4% per year among women. This improvement in survival reflects progress in diagnosing at an earlier stage and improvements in treatment. Discovering highly effective anticancer agents with low toxicity is a primary goal of cancer research.

The Myc family includes three major members, the proto-oncogene c-Myc (cellular Myelocytomatosis, short Myc), as well as L-myc and N-myc. These three Myc homologs are involved in the early stages of carcinogenesis and metastatic spread in most human cancers. In most types of tumors Myc gene is not mutated or duplicated, but its mRNA and/or protein levels are increased, indicating that in cancer Myc overexpression is induced at the level of transcription, mRNA steady state levels and translation. Indeed, myc gene expression normally depends on growth factor signaling and both myc mRNA and Myc protein have very short half-lives (of 30 and 20 min respectively) [Dang, C. V. (2012). MYC on the path to cancer. Cell 149, 22-35]. In tumor cells however, the cellular levels of Myc become independent from such signaling and regulation, and the resulting exacerbated Myc function drives intracellular and extracellular transcription programs that allow tumors to grow and thrive. However, Myc does not necessarily need to be overexpressed in order for a cancer to be highly dependent upon its activity. A study from Soucek et al. (Nature (2008) 455(7213):679-83) shows that tumors that express c-Myc at endogenous levels exhibit tumor regression upon Myc inhibition via a genetically engineered system. Therefore, treatment with a Myc inhibitor is not necessarily limited to cancers that overexpress Myc. Compounds according to this invention may also be used to regulate the translation of Myc mRNA, wherein the direct target for the compounds is a protein or RNA which regulate Myc mRNA translation, and as such any tumor which is Myc dependent will benefit from the therapeutic utility of these compounds.

Due to its extensive pathogenic significance, MYC is an important anticancer target. Deregulated Myc gene is found in a wide range of human hematological malignancies and solid tumors, especially in breast cancer, ovarian carcinoma, acute myeloid leukemia, chronic myelogenous leukemia, Hodgkin's and Burkitt's lymphoma, diffuse large Bcell lymphoma, prostate cancer, colon cancer, gastric cancer, primary central nervous system lymphoma, glioblastoma, medulloblastoma, melanoma, non-small cell lung carcinoma, germinal center-derived lymphomas, esophageal squamous cell carcinoma, osteosarcoma, bladder cancer, pancreatic cancer and lung adenocarcinoma. Recent studies also indicate that deregulation of c-MYC is related to the occurrence of BRAF V600E thyroid cancers, choroid plexus carcinoma, and colitis-associated cancer. In addition, amplification of the MYC gene was found in a significant number of epithelial ovarian cancer cases. In TCGA datasets, the amplification of Myc occurs in several cancer types, including breast, colorectal, pancreatic, gastric, and uterine cancers.

Although Myc gene is a very important oncogene and considered as a driver in carcinogenesis and MYC protein is a key transcription factor broadly targeting various genes, rational designing a direct Myc inhibitor is still challenging. This is mainly because MYC protein lacks structural regions amenable to therapeutic inhibition by small molecules and is considered an undruggable target [BioDrugs (2019) 33:539-553].

Designing and developing MYC modulators is challenging, primarily because the MYC protein has a disordered structure which lacks a pocket or groove that can act as a binding site for modulators. Interfering with the MYC transcription, blocking the protein-protein interaction (PPI) of MYC and its cofactors, and influencing on signaling pathways related to MYC were used in the past as potential modulatory targets, but failed to be developed as drug candidates. Myc PPI inhibitors failed to show sufficient efficacy in cell-based assays and animal models due to the requirement of high target occupancy to drive efficacy. Modulators of signaling pathways upstream to myc, for example mTOR modulators, failed due lack of target specificity.

Nevertheless, a therapeutic approach to target c-Myc has remained elusive. The absence of a clear ligand-binding domain establishes a formidable obstacle toward direct inhibition, which is a challenging feature shared among many compelling transcriptional targets in cancer. Thus, alternative modalities that target Myc are required, as outlined herein, namely compounds which regulate Myc mRNA translation.

### SUMMARY OF THE INVENTION

This invention provides a compound or its pharmaceutically acceptable salt, stereoisomer, tautomer, hydrate, N-oxide, reverse amide analog, prodrug, isotopic variants (e.g., deuterated analog), PROTAC, pharmaceutical product or any combination thereof, represented by the structure of formula I, II and I(a)-I(i), and by the structures listed in Table 1, as defined herein below. In various embodiments, the compound is a c-MYC mRNA translation modulator. In various embodiments, the compound is a c-MYC mRNA transcription regulator. In various embodiments, the compound is a c-MYC inhibitor. In various embodiments, the compound is any combination of a c-MYC mRNA transcription regulator, c-MYC mRNA transcription regulator and c-MYC inhibitor.

This invention further provides a pharmaceutical composition comprising a compound or its pharmaceutically acceptable salt, stereoisomer, tautomer, hydrate, N-oxide, prodrug, isotopic variants (e.g., deuterated analog), PROTAC, pharmaceutical product or any combination thereof, represented by the structure of formula I, II and I(a)-I(i), and by the structures listed in Table 1, as defined herein below, and a pharmaceutically acceptable carrier.

This invention further provides a method of treating, suppressing, reducing the severity, reducing the risk of developing or inhibiting cancer in a subject, comprising administering a compound represented by the structure of formula I, II and I(a)-I(i), and by the structures listed in Table 1, as defined herein below, to a subject suffering from cancer under conditions effective to treat, suppress, reduce the severity, reduce the risk of developing, or inhibit cancer in said subject.

This invention further provides a method for suppressing, reducing or inhibiting tumor growth in a subject, comprising administering a compound represented by the structure of formula I, II and I(a)-I(i), and by the structures listed in Table 1, as defined herein below, to a subject, under conditions effective to suppress, reduce or inhibit tumor growth in said subject. In some embodiment, the tumor is cancerous. In some embodiment, the subject suffers from cancer.

This invention further provides a method of modulating c-MYC mRNA translation in a cell, comprising contacting a compound represented by the structure of formula I, II and I(a)-I(i) and by the structures listed in Table 1, as defined herein below, with a cell, thereby modulating c-MYC mRNA translation in said cell.

This invention further provides a method of regulating c-MYC mRNA transcription in a cell, comprising contacting a compound represented by the structure of formula I, II and I(a)-I(i) and by the structures listed in Table 1, as defined herein below, with a cell, thereby regulating c-MYC mRNA transcription in said cell.

### BRIEF DESCRIPTION OF THE DRAWINGS

The patent of application file contains at least one drawing executed in color. Copies of this patent or patent application publication with color drawing(s) will be provided by the office upon request and payment of the necessary fee.

FIG. 1 demonstrates how Protein Synthesis Monitoring (PSM) specifically monitors c-Myc synthesis. The assay system comprises human non-small cell lung carcinoma cell line A549, which is expressing high level of c-Myc. Two tRNAs (di-tRNA) which decode one specific glutamine codon and one specific serine codon were transfected with control RNAi or an RNAi directed to c-Myc. The FRET signal specifically monitors c-Myc translation, as the FRET signal in c-Myc siRNA treated cells was inhibited. In blue, cell nuclei stained with DAPI; in yellow, FRET signals from tRNA pair which decodes glutamine-serine di-codons.

FIG. 2 depicts selective regulation of c-Myc translation. The panel demonstrates metabolic labeling in A549 cells, treated with vehicle, general translation inhibitor cycloheximide or anti-c-Myc compound. Treatment with cycloheximide resulted in total inhibition of global protein synthesis, while treatment with tested compound showed no significant effect. In gray, cell nuclei stained with DAPI; in yellow, L-Azidohomoalanine (AHA) metabolic labeling.

FIG. 3 demonstrates that compounds act at the level of mRNA processing/stability. A549 cells were exposed to vehicle, general transcription inhibitor actinomycin D or anti-c-Myc compound. In the upper panel, significant decrease in c-Myc protein level was observed after treatment with either actinomycin D or tested compound. Lower panel shows complete reduction in c-Myc mRNA level as well as transcription sites after treatment with actinomycin D. Treatment with tested compound although reduced c-Myc mRNA levels by 30% without affecting transcription sites. In gray, cell nuclei stained with DAPI; in red, c-Myc protein; in purple, c-Myc mRNA; in yellow, c-Myc transcription sites.

FIG. 4 demonstrates the efficacy of compounds according to this invention in A549 cells.

FIG. 5 demonstrates the in vivo data measured for compound 332. Compound 332 inhibited c-Myc-dependent tumor growth in-vivo. Relative tumor volumes of A549 xenografts in NMRI female nude mice after treatment with compound 3 mg/kg twice a week for 49 days. Error bars represent median±SEM, n=10 mice at each time point and analyzed by one-tailed T-TEST in Prism for *p<0.05

### DETAILED DESCRIPTION OF THE INVENTION

In various embodiments, this invention is directed to a compound represented by the structure of formula (I):

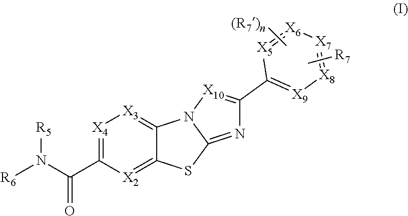

wherein

X₂, X₃, and X₄, are each independently nitrogen or CH;

X₅, X₆, X₇, X₈ and X₉ are each independently nitrogen or carbon atoms;

X₁₀ is N, CH, or C(R) (e.g., C(NH—CH₂-cyclopropyl), C(CH₃), C(cyclopropyl), C(isopropoxy));

R₅ is H or C₁-C₅ linear or branched alkyl (e.g. methyl);

R₆ is H, F, Cl, Br, I, OH, SH, R₈—OH, R₈—SH, —R₈—O—R₁₀ (e.g., CH₂—O—CH₃, (CH₂)₂—O—CH₃(CH₂)₃—O—CH₃, (CH₂)₂—O—CH(CH₃)₂), R₈—S—R₁₀ (e.g., (CH₂)₃—S—(CH₂)₂CH₃), R₈—NHC(O)—R₁₀, —O—R₈—R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-tetrahydrofurane, CH₂-oxa-azaspirodecane, CH₂-azaspiroheptane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₃—N(CH₂CH₃)₂, (CH₂)₃—N(CH(CH₃)₂)₂, (CH₂)₃-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂—C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃-OCH₃, (CH₂)₂-OCH₃, (CH₂)₂-OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclobutyl, cyclohexyl, methoxycyclopropyl, methylcyclobutyl, cyclopropyl, aminomethyl-cyclobutyl, methoxycyclobutyl, 2,3-dihydro-1H-indenol), R₈-(substituted or unsubstituted C₃-C₈ cycloalkyl), substituted or unsubstituted 3-8 membered heterocyclic ring (e.g., piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;

or R₆ and R₅ are joined to form a substituted or unsubstituted 5-8 membered heterocyclic ring (e.g., azepane, piperazine, 2-(piperazin-1-yl)acetamide;

or R₆ is represented by the structure of formula B or Bi:

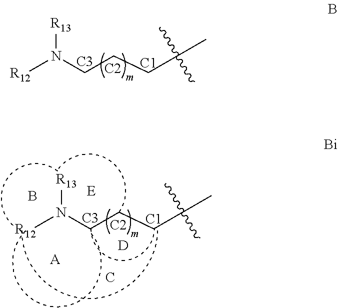

wherein

m is 0 or 1; and
R₁₂ is R₂₀ or C₁-C₅ C(O)-alkyl, and R₁₃ is R₃₀; or
R₁₂ and R₁₃ are both H;
R₁₂ and R₁₃ are each independently H or substituted or unsubstituted C₁-C₅ alkyl (e.g., ethyl, trifluoroethyl);
R₁₂ and C3 are joined to form ring A and R₁₃ is R₃₀; or
R₁₂ and R₁₃ are joined to form ring B; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
   Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine;
   Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
   Ring D is a substituted or unsubstituted C₃-C₈ cycloalkyl (e.g., cyclobutane, cyclohexane);

R₇ is H, F, Cl, Br, I, OH, O—R₂₀, SH, R₈—OH, R₈—SH, SR₁₀, —R₈—O—R₁₀, —R₈—S—R₁₀, R₈—(C₃-C₈ cycloalkyl), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)), C(O)N(R₁₀)(R₁₁) (e.g., C(O)NH(CH₃), C(O)NH(CH₂CH₂OCH₃), C(O)NH(CH₂CH₂OH)), SO₂R, SO₂N(R₁₀)(R₁₁), CH(CF₃)(NH—R₁₀), C₁-C₅ linear or branched, substituted or unsubstituted alkyl (e.g., methylimidazole, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy, ethoxy), optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopropanol, cyclohexyl), substituted or unsubstituted 4-7 membered heterocyclic ring (e.g., morpholine, tetrahydrofuran, tetrahydropyran, oxetane, oxetan-3-ol, pyrrolidine, 1-methylpyrrolidine, pyrrolidin-2-one, pyrrolidinone, imidazole, pyrazole, piperazine, piperidine, piperidine-4-carbonitrile, 4-fluoropiperidine, oxadiazole, triazole, 2-oxopyrrolidine), R₈-(substituted or unsubstituted single, fused or spiro 3-8 membered heterocyclic ring), substituted or unsubstituted aryl, substituted or unsubstituted benzyl;

or R₇ is represented by the structure of formula A:

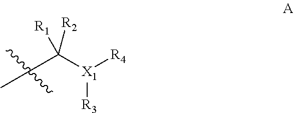

wherein
X₁ is N or O;
R₁ and R₂ are each independently H, F, or CF₃; or
R₁ and R₂ are joined to form ═O or a C₃-C₈ carbocyclic or heterocyclic ring (e.g., cyclopropyl);
R₃ and R₄ are each independently H, Me, substituted or unsubstituted C₁-C₅ alkyl (e.g., methoxyethylene, methylaminoethylene, aminoethylene), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀
R₃ and R₄ are joined to form a 3-8 membered heterocyclic ring (e.g., pyrrolidine, 2-oxopyrrolidine, piperidine, morpholine, piperazine, imidazole);
   wherein if X₁ is O then R₄ is absent;

R₇′ is H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted 3-8 membered heterocyclic ring (e.g., morpholine, pyran, oxetane, pyrrolidine, imidazole, piperazine, piperidine, diaoxazole, 2-oxopyrrolidine), substituted or unsubstituted aryl, substituted or unsubstituted benzyl;
or R₇ and R₇′ are joined to form a 5 or 6 membered substituted or unsubstituted, saturated, unsaturated or aromatic, carbocyclic or heterocyclic ring (e.g., piperidine, pyrrolidine, tetrahydrofuran, tetrahydropyran);

R₂₀ is represented by the following structure:

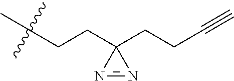 [SMILES: *CCC1(CCC#C)N=N1]

R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

R is H, F, Cl, Br, I, OH, SH, OH, alkoxy, NH(R₁₀), NH—CH₂-cyclopropyl, N(R₁₀)(R₁₁), CF₃, CN, NO₂, COOH, C₁-C₅ linear or branched, substituted or unsubstituted alkyl (e.g., methyl, ethyl, CH₂—OH, CH₂—CH₂—OH, CH₂—CH₂—O—CH₂—CH₂—O—CH₃, CH₂—O—CH₂—CH₂—O—CH₃), C₃-C₈ substituted or unsubstituted cycloalkyl, cyclopropyl, C₁-C₅ linear or branched alkoxy, isopropoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

each R₈ is independently [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂-cyclopropyl, CH₂—CH₂—O—CH₃), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;

or R₁₀ and R₁₁ are joined to form a substituted or unsubstituted 3-8 membered heterocyclic ring (e.g., piperazine, piperidine),

n is an integer between 0 and 4 (e.g., 1, 2);

or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In various embodiments, this invention is directed to a compound represented by the structure of formula I(a):

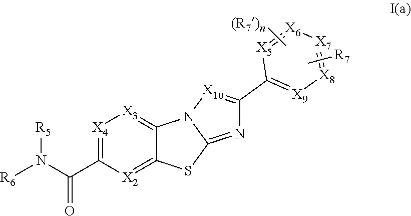

wherein

X₂, X₃, and X₄, are each independently nitrogen or CH;

X₅, X₆, X₇, X₈ and X₉ are each independently nitrogen or carbon atoms;

X₁₀ is N, CH, or C(R) (e.g., C(NH—CH₂-cyclopropyl), C(CH₃), C(cyclopropyl), C(isopropoxy));

R₅ is H or C₁-C₅ linear or branched alkyl (e.g. methyl);

R₆ is H, F, Cl, Br, I, OH, SH, R₈—OH, R₈—SH, —R₈—O—R₁₀ (e.g., CH₂—O—CH₃, (CH₂)₂O—CH₃ (CH₂)₃O—CH₃, (CH₂)₂O—CH(CH₃)₂), R₈—S—R₁₀ (e.g., (CH₂)₃-S—(CH₂)₂CH₃), R₈—NHC(O)—R₁₀, —O—R₈—R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-tetrahydrofurane, CH₂-oxa-azaspirodecane, CH₂-azaspiroheptane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₃—N(CH₂CH₃)₂, (CH₂)₃—N(CH(CH₃)₂)₂, (CH₂)₃-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂—C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃-OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclobutyl, cyclohexyl, methoxycyclopropyl, methylcyclobutyl, cyclopropyl, aminomethyl-cyclobutyl, methoxycyclobutyl, 2,3-dihydro-1H-indenol), R₈-(substituted or unsubstituted C₃-C₈ cycloalkyl), substituted or unsubstituted 3-8 membered heterocyclic ring (e.g., piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;

or R₆ and R₅ are joined to for a substituted or unsubstituted 5-8 membered heterocyclic ring (e.g., azepane, piperazine, 2-(piperazin-1-yl)acetamide;

or R₆ is represented by the structure of formula B or Bi:

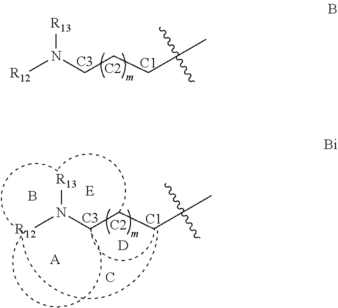

wherein

m is 0 or 1; and
R₁₂ is R₂₀ or C₁-C₅ C(O)-alkyl, and R₁₃ is R₃₀; or
R₁₂ and R₁₃ are both H;
R₁₂ and R₁₃ are each independently H or substituted or unsubstituted C₁-C₅ alkyl (e.g., ethyl, trifluoroethyl);
R₁₂ and C3 are joined to form ring A and R₁₃ is R₃₀; or
R₁₂ and R₁₃ are joined to form ring B; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
   Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
   Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
   Ring D is a substituted or unsubstituted C₃-C₈ cycloalkyl (e.g., cyclobutane, cyclohexane);

R₇ is O—R₂₀, SH, R₈—OH, R₈—SH, SR₁₀, —R₈—O—R₁₀, —R₈—S—R₁₀, R₈—(C₃-C₈ cycloalkyl), CD₃, OCD₃, NO₂, —CH₂CN, —R₈CN, R₈—N(R₁₀)(R₁₁), R₉—R₈—N(R₁₀)(R₁₁), B(OH)₂, —OC(O)CF₃, —OCH₂Ph, NHCO—N(R₁₀)(R₁₁), R₈—C(O)—R₁₀, SO₂N(R₁₀)(R₁₁), CH(CF₃)(NH—R₁₀), C₁-C₅ linear or branched, substituted or unsubstituted alkenyl, C₁-C₅ linear or branched thioalkoxy, C₁-C₅ linear or branched alkoxyalkyl, substituted or unsubstituted 4-7 membered heterocyclic ring (e.g., morpholine, tetrahydrofuran, tetrahydropyran, oxetane, oxetan-3-ol, pyrrolidine, 1-methylpyrrolidine, pyrrolidin-2-one, pyrrolidinone, imidazole, pyrazole, piperazine, piperidine, piperidine-4-carbonitrile, 4-fluoropiperidine, oxadiazole, triazole, pyrazole, 2-oxopyrrolidine), substituted or unsubstituted aryl, substituted or unsubstituted benzyl, R₈-(substituted or unsubstituted single, fused or spiro 3-8 membered heterocyclic ring);

or R₇ is represented by the structure of formula A:

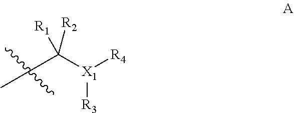

wherein
X₁ is N or O; R₁ and R₂ are each independently H, F, or CF₃; or R₁ and R₂ are joined to form a C₃-C₈ carbocyclic or heterocyclic ring (e.g., cyclopropyl);
R₃ and R₄ are each independently H, Me, substituted or unsubstituted C₁-C₅ alkyl (e.g., methoxyethylene, methylaminoethyl, aminoethyl), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀; or
R₃ and R₄ are joined to form a 3-8 membered heterocyclic ring (e.g., pyrrolidine, 2-oxopyrrolidine, piperidine, morpholine, piperazine, imidazole);
   wherein if X₁ is O then R₄ is absent;

R₇′ is H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted 3-8 membered heterocyclic ring (e.g., morpholine, pyran, oxetane, pyrrolidine, imidazole, piperazine, piperidine, diaoxazole, 2-oxopyrrolidine), substituted or unsubstituted aryl, substituted or unsubstituted benzyl;
or R₇ and R₇′ are joined to form a 5 or 6 membered substituted or unsubstituted, saturated, unsaturated or aromatic, carbocyclic or heterocyclic ring (e.g., piperidine, pyrrolidine, tetrahydrofuran, tetrahydropyran);

R₂₀ is represented by the following structure:

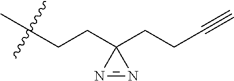 [SMILES: *CCC1(CCC#C)N=N1]

R is H, F, Cl, Br, I, OH, SH, OH, alkoxy, NH(R₁₀), NH—CH₂-cyclopropyl, N(R₁₀)(R₁₁), CF₃, CN, NO₂, COOH, C₁-C₅ linear or branched, substituted or unsubstituted alkyl (e.g., methyl, ethyl, CH₂—OH, CH₂—CH₂—OH, CH₂—CH₂—O—CH₂—CH₂—O—CH₃, CH₂—O—CH₂—CH₂—O—CH₃), C₃-C₈ substituted or unsubstituted cycloalkyl, cyclopropyl, C₁-C₅ linear or branched alkoxy, isopropoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

each R₈ is independently [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂-cyclopropyl, CH₂—CH₂—O—CH₃), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;

or R₁₀ and R₁₁ are joined to form a substituted or unsubstituted 3-8 membered heterocyclic ring (e.g., piperazine, piperidine),

n is an integer between 0 and 4 (e.g., 1, 2);

or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In various embodiments, this invention is directed to a compound represented by the structure of formula I(b):

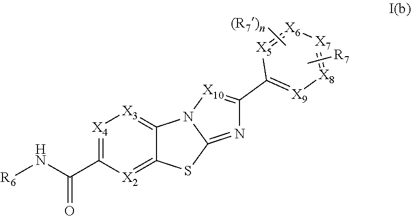

wherein

X₂, X₃, and X₄, are each independently nitrogen or CH;

X₅, X₆, X₇, X₈ and X₉ are each independently nitrogen or carbon atoms;

X₁₀ is N, CH, or C(R) (e.g., C(NH—CH₂-cyclopropyl), C(CH₃), C(cyclopropyl), C(isopropoxy));

R₆ is F, Cl, Br, I, OH, SH, R₈—OH, R₈—SH, —R₈—O—R₁₀ (e.g., CH₂—O—CH₃), R₈—S—R₁₀ (e.g., (CH₂)₃-S—(CH₂)₂CH₃), R₈—NHC(O)—R₁₀, —O—R₈—R₁₀, R₈-(substituted or unsubstituted C₃-C₈ cycloalkyl) (e.g., CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), (CH₂)₃-pyran, CH₂-tetrahydrofurane, CH₂-dioxane, CH₂-methyl-THF, CH₂-tetrahydrofurane, CH₂-oxa-azaspirodecane, CH₂-azaspiroheptane, (CH₂)₃-dimethylpyrazole, CH₂-methyl-azetidine, CH₂-azaspiroheptane, CF₃, CD₃, OCD₃, CN, NO₂, —CH₂CN, —R₈CN, NH₂, NHR, N(R)₂, NH(R₁₀), N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃-OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., methoxycyclopropyl, methylcyclobutyl, cyclopropyl, aminomethyl-cyclobutyl, methoxycyclobutyl, 2,3-dihydro-1H-indenol), substituted or unsubstituted 3-8 membered heterocyclic ring (e.g., trifluoromethyl-oxetane, hydroxy-tetrahydrofurane, 1-methylazepan-2-one, 3-azabicyclo[3.1.0]hexane), substituted or unsubstituted aryl, substituted or unsubstituted benzyl;

or R₆ and R₅ are joined to for a substituted or unsubstituted 5-8 membered heterocyclic ring (e.g., azepane, piperazine, 2-(piperazin-1-yl)acetamide;

or R₆ is represented by the structure of formula B or Bi:

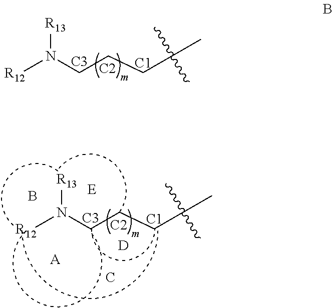

wherein

m is 0 or 1; and
R₁₂ is R₂₀ or C₁-C₅ C(O)-alkyl, and R₁₃ is R₃₀; or
R₁₂ and R₁₃ are both H; or
R₁₂ and C3 are joined to form ring A and R₁₃ is R₃₀; or
R₁₂ and R₁₃ are joined to form a substituted or unsubstituted pyrrolidine ring, piperazine, thiomorpholine 1,1-dioxide, 2-oxa-6-azaspiro[3.3]heptane, pyrazole, imidazole, 2,5-diazabicyclo[2.2.1]heptane or a diazabicyclo[2.2.1]heptane; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
   Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
   Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
   Ring D is a substituted or unsubstituted C₃-C₈ cycloalkyl (e.g., cyclobutane, cyclohexane);

R₇ is H, F, Cl, Br, I, OH, O—R₂₀, SH, R₈—OH, R₈—SH, SR₁₀, —R₈—O—R₁₀, —R₈—S—R₁₀, R₈—(C₃-C₈ cycloalkyl), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)), C(O)N(R₁₀)(R₁₁) (e.g., C(O)NH(CH₃), C(O)NH(CH₂CH₂OCH₃), C(O)NH(CH₂CH₂OH)), SO₂R, SO₂N(R₁₀)(R₁₁), CH(CF₃)(NH—R₁₀), C₁-C₅ linear or branched, substituted or unsubstituted alkyl (e.g., methylimidazole, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopropanol, cyclohexyl), substituted or unsubstituted 4-7 membered heterocyclic ring (e.g., morpholine, tetrahydrofuran, tetrahydropyran, oxetane, oxetan-3-ol, pyrrolidine, 1-methylpyrrolidine, pyrrolidin-2-one, pyrrolidinone, imidazole, pyrazole, piperazine, piperidine, piperidine-4-carbonitrile, 4-fluoropiperidine, oxadiazole, triazole, 2-oxopyrrolidine), R₈-(substituted or unsubstituted single, fused or spiro 3-8 membered heterocyclic ring), substituted or unsubstituted aryl, substituted or unsubstituted benzyl;

or R₇ is represented by the structure of formula A:

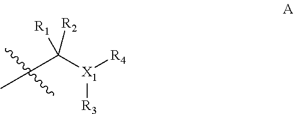

wherein
X₁ is N or O;
R₁ and R₂ are each independently H, F, or CF₃; or
R₁ and R₂ are joined to form ═O or a C₃-C₈ carbocyclic or heterocyclic ring (e.g., cyclopropyl);
R₃ and R₄ are each independently H, Me, substituted or unsubstituted C₁-C₅ alkyl (e.g., methoxyethylene, methylaminoethyl, aminoethyl), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀
R₃ and R₄ are joined to form a 3-8 membered heterocyclic ring (e.g., pyrrolidine, 2-oxopyrrolidine, piperidine, morpholine, piperazine, imidazole);
wherein if X₁ is O then R₄ is absent;

R₇′ is H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted 3-8 membered heterocyclic ring (e.g., morpholine, pyran, oxetane, pyrrolidine, imidazole, piperazine, piperidine, diaoxazole, 2-oxopyrrolidine), substituted or unsubstituted aryl, substituted or unsubstituted benzyl;
or R₇ and R₇′ are joined to form a 5 or 6 membered substituted or unsubstituted, saturated, unsaturated or aromatic, carbocyclic or heterocyclic ring (e.g., piperidine, pyrrolidine, tetrahydrofuran, tetrahydropyran);

R₂₀ is represented by the following structure:

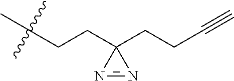 [SMILES: *CCC1(CCC#C)N=N1]

R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

R is H, F, Cl, Br, I, OH, SH, OH, alkoxy, NH(R₁₀), NH—CH₂-cyclopropyl, N(R₁₀)(R₁₁), CF₃, CN, NO₂, COOH, C₁-C₅ linear or branched, substituted or unsubstituted alkyl (e.g., methyl, ethyl, CH₂—OH, CH₂—CH₂—OH, CH₂—CH₂—O—CH₂—CH₂—O—CH₃, CH₂—O—CH₂—CH₂—O—CH₃), C₃-C₈ substituted or unsubstituted cycloalkyl, cyclopropyl, C₁-C₅ linear or branched alkoxy, isopropoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

each R₈ is independently [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂-cyclopropyl, CH₂—CH₂—O—CH₃), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;

or R₁₀ and R₁₁ are joined to form a substituted or unsubstituted 3-8 membered heterocyclic ring (e.g., piperazine, piperidine),

n is an integer between 0 and 4 (e.g., 1, 2);

or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In various embodiments, this invention is directed to a compound represented by the structure of formula I(c):

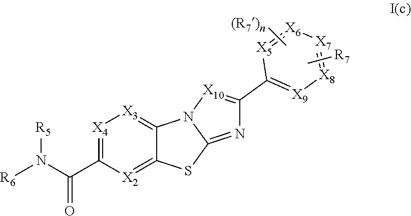

wherein

X₂, X₃, and X₄, are each independently nitrogen or CH;

X₅, X₆, X₇, X₈ and X₉ are each independently nitrogen or carbon atoms;

X₁₀ is N, CH, or C(R) (e.g., C(NH—CH₂-cyclopropyl), C(CH₃), C(cyclopropyl), C(isopropoxy));

R₈ is H or C₁-C₅ linear or branched alkyl (e.g. methyl);

R₆ is H, F, Cl, Br, I, OH, SH, R₈—OH, R₈—SH, —R₈—O—R₁₀ (e.g., CH₂—O—CH₃, (CH₂)₂—O—CH₃ (CH₂)₃—O—CH₃, (CH₂)₂—O—CH(CH₃)₂), R₈—S—R₁₀ (e.g., (CH₂)₃—S—(CH₂)₂CH₃), R₈—NHC(O)—R₁₀, —O—R₈—R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-tetrahydrofurane, CH₂-oxa-azaspirodecane, CH₂-azaspiroheptane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₃—N(CH₂CH₃)₂, (CH₂)₃—N(CH(CH₃)₂)₂, (CH₂)₃-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂—C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclobutyl, cyclohexyl, methoxycyclopropyl, methylcyclobutyl, cyclopropyl, aminomethyl-cyclobutyl, methoxycyclobutyl, 2,3-dihydro-1H-indenol), R₈-(substituted or unsubstituted C₃-C₈ cycloalkyl), substituted or unsubstituted 3-8 membered heterocyclic ring (e.g., piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;

or R₆ and R₅ are joined to form a substituted or unsubstituted 5-8 membered heterocyclic ring (e.g., azepane, piperazine, 2-(piperazin-1-yl)acetamide;

or R₆ is represented by the structure of formula B or Bi:

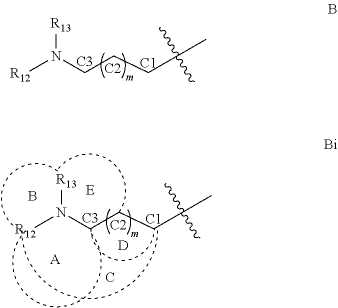

wherein

m is 0 or 1; and
R₁₂ is R₂₀ or C₁-C₅ C(O)-alkyl, and R₁₃ is R₃₀; or
R₁₂ and R₁₃ are both H;
R₁₂ and R₁₃ are each independently H or substituted or unsubstituted C₁-C₅ alkyl (e.g., ethyl, trifluoroethyl);
R₁₂ and C3 are joined to form ring A and R₁₃ is R₃₀; or
R₁₂ and R₁₃ are joined to form ring B; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
   Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
   Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
   Ring D is a substituted or unsubstituted C₃-C₈ cycloalkyl (e.g., cyclobutane, cyclohexane);
R₇ is Br, I, OH, O—R₂₀, SH, R₈—OH, R₈—SH, SR₁₀, —R₈—O—R₁₀, —R₈—S—R₁₀, R₈—(C₃-C₈ cycloalkyl), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)), C(O)N(R₁₀)(R₁₁) (e.g., C(O)NH(CH₃), C(O)NH(CH₂CH₂OCH₃), C(O)NH(CH₂CH₂OH)), SO₂R, SO₂N(R₁₀)(R₁₁), CH(CF₃)(NH—R₁₀), C₁-C₅ linear or branched, substituted or unsubstituted alkyl (e.g., methylimidazole, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched thioalkyl, C₁-C₅ linear or branched thioalkoxy, C₁-C₅ linear or branched alkoxyalkyl, substituted or unsubstituted C₃-C₈ cycloalkyl (e.g., cyclopropyl, cyclopropanol, cyclohexyl), substituted or unsubstituted 4-7 membered heterocyclic ring (e.g., morpholine, tetrahydrofuran, tetrahydropyran, oxetane, oxetan-3-ol, pyrrolidine, 1-methylpyrrolidine, pyrrolidin-2-one, pyrrolidinone, imidazole, pyrazole, piperazine, piperidine, piperidine-4-carbonitrile, 4-fluoropiperidine, oxadiazole, triazole, 2-oxopyrrolidine), R₈-(substituted or unsubstituted single, fused or spiro 3-8 membered heterocyclic ring), substituted or unsubstituted aryl, substituted or unsubstituted benzyl;

or R₇ is represented by the structure of formula A:

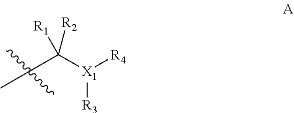

wherein
X₁ is N or O;
R₁ and R₂ are each independently H, F, or CF₃; or
R₁ and R₂ are joined to form ═O or a C₃-C₈ carbocyclic or heterocyclic ring (e.g., cyclopropyl);
R₃ and R₄ are each independently H, Me, substituted or unsubstituted C₁-C₅ alkyl (e.g., methoxyethylene, methylaminoethyl, aminoethyl), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀
R₃ and R₄ are joined to form a 3-8 membered heterocyclic ring (e.g., pyrrolidine, 2-oxopyrrolidine, piperidine, morpholine, piperazine, imidazole);
wherein if X₁ is O then R₄ is absent;
R₇′ is F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted 3-8 membered heterocyclic ring (e.g., morpholine, pyran, oxetane, pyrrolidine, imidazole, piperazine, piperidine, diaoxazole, 2-oxopyrrolidine), substituted or unsubstituted aryl, substituted or unsubstituted benzyl;

wherein R₇′ is different than R₇;
or R₇ and R₇′ are joined to form a 5 or 6 membered substituted or unsubstituted, saturated, unsaturated or aromatic, carbocyclic or heterocyclic ring (e.g., piperidine, pyrrolidine, tetrahydrofuran, tetrahydropyran);

R₂₀ is represented by the following structure:

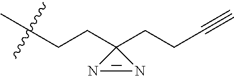 [SMILES: *CCC1(CCC#C)N=N1]

R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

R is H, F, Cl, Br, I, OH, SH, OH, alkoxy, NH(R₁₀), NH—CH₂-cyclopropyl, N(R₁₀)(R₁₁), CF₃, CN, NO₂, COOH, C₁-C₅ linear or branched, substituted or unsubstituted alkyl (e.g., methyl, ethyl, CH₂—OH, CH₂—CH₂—OH, CH₂—CH₂—O—CH₂—CH₂—O—CH₃, CH₂—O—CH₂—CH₂—O—CH₃), C₃-C₈ substituted or unsubstituted cycloalkyl, cyclopropyl, C₁-C₅ linear or branched alkoxy, isopropoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

each R₈ is independently [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂-cyclopropyl, CH₂—CH₂—O—CH₃), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;

or R₁₀ and R₁₁ are joined to form a substituted or unsubstituted 3-8 membered heterocyclic ring (e.g., piperazine, piperidine),

n is an integer between 1 and 4 (e.g., 1, 2);

or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In various embodiments, this invention is directed to a compound represented by the structure of formula I(d):

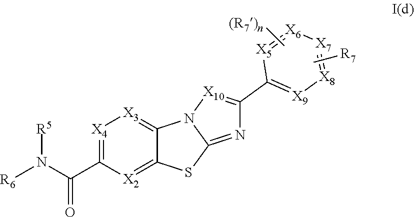

wherein

X₂, X₃, and X₄, are each independently nitrogen or CH;

X₅, X₆, X₇, X₈ and X₉ are each independently nitrogen or carbon atoms;

X₁₀ is N, CH, or C(R) (e.g., C(NH—CH₂-cyclopropyl), C(CH₃), C(cyclopropyl), C(isopropoxy));

wherein at least one of X₂, X₃, X₄, X₅, X₆, X₇, X₈, X₉ or X₁₀ is N;

R₅ is H or C₁-C₅ linear or branched alkyl (e.g. methyl);

R₆ is H, F, Cl, Br, I, OH, SH, R₈—OH, R₈—SH, —R₈—O—R₁₀ (e.g., CH₂—O—CH₃, (CH₂)₂—O—CH₃ (CH₂)₃—O—CH₃, (CH₂)₂—O—CH(CH₃)₂), R₈—S—R₁₀ (e.g., (CH₂)₃—S—(CH₂)₂CH₃), R₈—NHC(O)—R₁₀, —O—R₈—R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-tetrahydrofurane, CH₂-oxa-azaspirodecane, CH₂-azaspiroheptane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₃—N(CH₂CH₃)₂, (CH₂)₃—N(CH(CH₃)₂)₂, (CH₂)₃-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂—C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclobutyl, cyclohexyl, methoxycyclopropyl, methylcyclobutyl, cyclopropyl, aminomethyl-cyclobutyl, methoxycyclobutyl, 2,3-dihydro-1H-indenol), R₈-(substituted or unsubstituted C₃-C₈ cycloalkyl), substituted or unsubstituted 3-8 membered heterocyclic ring (e.g., piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;

or R₆ and R₅ are joined to form a substituted or unsubstituted 5-8 membered heterocyclic ring (e.g., azepane, piperazine, 2-(piperazin-1-yl)acetamide;

or R₆ is represented by the structure of formula B or Bi:

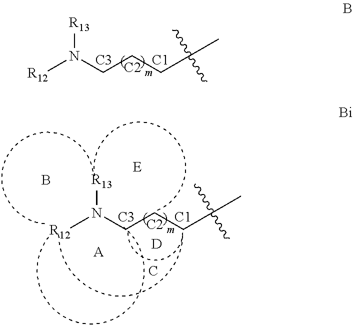

wherein

m is 0 or 1; and
R₁₂ is R₂₀ or C₁-C₅ C(O)-alkyl, and R₁₃ is R₃₀; or
R₁₂ and R₁₃ are both H;
R₁₂ and R₁₃ are each independently H or substituted or unsubstituted C₁-C₅ alkyl (e.g., ethyl, trifluoroethyl);
R₁₂ and C3 are joined to form ring A and R₁₃ is R₃₀; or
R₁₂ and R₁₃ are joined to form ring B; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
   Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
   Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
   Ring D is a substituted or unsubstituted C₃-C₈ cycloalkyl (e.g., cyclobutane, cyclohexane);

R₇ is H, F, Cl, Br, I, OH, O—R₂₀, SH, R₈—OH, R₈—SH, SR₁₀, —R₈—O—R₁₀, —R₈—S—R₁₀, R₈—(C₃-C₈ cycloalkyl), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)), C(O)N(R₁₀)(R₁₁) (e.g., C(O)NH(CH₃), C(O)NH(CH₂CH₂OCH₃), C(O)NH(CH₂CH₂OH)), SO₂R, SO₂N(R₁₀)(R₁₁), CH(CF₃)(NH—R₁₀), C₁-C₅ linear or branched, substituted or unsubstituted alkyl (e.g., methylimidazole, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy, ethoxy), optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopropanol, cyclohexyl), substituted or unsubstituted 4-7 membered heterocyclic ring (e.g., morpholine, tetrahydrofuran, tetrahydropyran, oxetane, oxetan-3-ol, pyrrolidine, 1-methylpyrrolidine, pyrrolidin-2-one, pyrrolidinone, imidazole, pyrazole, piperazine, piperidine, piperidine-4-carbonitrile, 4-fluoropiperidine, oxadiazole, triazole, 2-oxopyrrolidine), R₈-(substituted or unsubstituted single, fused or spiro 3-8 membered heterocyclic ring), substituted or unsubstituted aryl, substituted or unsubstituted benzyl;

or R₇ is represented by the structure of formula A:

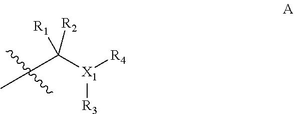

wherein
X₁ is N or O;
R₁ and R₂ are each independently H, F, or CF₃; or
R₁ and R₂ are joined to form ═O or a C₃-C₈ carbocyclic or heterocyclic ring (e.g., cyclopropyl);
R₃ and R₄ are each independently H, Me, substituted or unsubstituted C₁-C₅ alkyl (e.g., methoxyethylene, methylaminoethyl, aminoethyl), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀
R₃ and R₄ are joined to form a 3-8 membered heterocyclic ring (e.g., pyrrolidine, 2-oxopyrrolidine, piperidine, morpholine, piperazine, imidazole);
wherein if X₁ is O then R₄ is absent;
R₇′ is H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted 3-8 membered heterocyclic ring (e.g., morpholine, pyran, oxetane, pyrrolidine, imidazole, piperazine, piperidine, diaoxazole, 2-oxopyrrolidine), substituted or unsubstituted aryl, substituted or unsubstituted benzyl;
or R₇ and R₇′ are joined to form a 5 or 6 membered substituted or unsubstituted, saturated, unsaturated or aromatic, carbocyclic or heterocyclic ring (e.g., piperidine, pyrrolidine, tetrahydrofuran, tetrahydropyran);

R₂₀ is represented by the following structure:

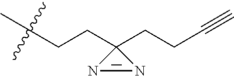 [SMILES: *CCC1(CCC#C)N=N1]

R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

R is H, F, Cl, Br, I, OH, SH, OH, alkoxy, NH(R₁₀), NH—CH₂-cyclopropyl, N(R₁₀)(R₁₁), CF₃, CN, NO₂, COOH, C₁-C₅ linear or branched, substituted or unsubstituted alkyl (e.g., methyl, ethyl, CH₂—OH, CH₂—CH₂—OH, CH₂—CH₂—O—CH₂—CH₂—O—CH₃, CH₂—O—CH₂—CH₂—O—CH₃), C₃-C₈ substituted or unsubstituted cycloalkyl, cyclopropyl, C₁-C₅ linear or branched alkoxy, isopropoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

each R₈ is independently [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂-cyclopropyl, CH₂—CH₂—O—CH₃), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;

or R₁₀ and R₁₁ are joined to form a substituted or unsubstituted 3-8 membered heterocyclic ring (e.g., piperazine, piperidine),

n is an integer between 0 and 4 (e.g., 1, 2);

or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In various embodiments, this invention is directed to a compound represented by the structure of formula I(e):

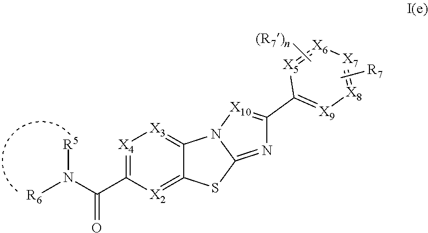

wherein

X₂, X₃, and X₄, are each independently nitrogen or CH;

X₅, X₆, X₇, X₈ and X₉ are each independently nitrogen or carbon atoms;

X₁₀ is N, CH, or C(R) (e.g., C(NH—CH₂-cyclopropyl), C(CH₃), C(cyclopropyl), C(isopropoxy));

R₅ and R₆ are joined to for a substituted or unsubstituted 5-8 membered heterocyclic ring (e.g., azepane, piperazine;

R₇ is H, F, Cl, Br, I, OH, O—R₂₀, SH, R₈—OH, R₈—SH, SR₁₀, —R₈—O—R₁₀, —R₈—S—R₁₀, R₈—(C₃-C₈ cycloalkyl), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)), C(O)N(R₁₀)(R₁₁) (e.g., C(O)NH(CH₃), C(O)NH(CH₂CH₂OCH₃), C(O)NH(CH₂CH₂OH)), SO₂R, SO₂N(R₁₀)(R₁₁), CH(CF₃)(NH—R₁₀), C₁-C₅ linear or branched, substituted or unsubstituted alkyl (e.g., methylimidazole, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy, ethoxy), optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopropanol, cyclohexyl), substituted or unsubstituted 4-7 membered heterocyclic ring (e.g., morpholine, tetrahydrofuran, tetrahydropyran, oxetane, oxetan-3-ol, pyrrolidine, 1-methylpyrrolidine, pyrrolidin-2-one, pyrrolidinone, imidazole, pyrazole, piperazine, piperidine, piperidine-4-carbonitrile, 4-fluoropiperidine, oxadiazole, triazole, 2-oxopyrrolidine), R₈-(substituted or unsubstituted single, fused or spiro 3-8 membered heterocyclic ring), substituted or unsubstituted aryl, substituted or unsubstituted benzyl;

or R₇ is represented by the structure of formula A:

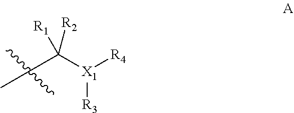

wherein
X₁ is N or O;
R₁ and R₂ are each independently H, F, or CF₃; or
R₁ and R₂ are joined to form ═O or a C₃-C₈ carbocyclic or heterocyclic ring (e.g., cyclopropyl);
R₃ and R₄ are each independently H, Me, substituted or unsubstituted C₁-C₅ alkyl (e.g., methoxyethylene, methylaminoethyl, aminoethyl), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀
R₃ and R₄ are joined to form a 3-8 membered heterocyclic ring (e.g., pyrrolidine, 2-oxopyrrolidine, piperidine, morpholine, piperazine, imidazole);
wherein if X₁ is O then R₄ is absent;
R₇′ is H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted 3-8 membered heterocyclic ring (e.g., morpholine, pyran, oxetane, pyrrolidine, imidazole, piperazine, piperidine, diaoxazole, 2-oxopyrrolidine), substituted or unsubstituted aryl, substituted or unsubstituted benzyl;
or R₇ and R₇′ are joined to form a 5 or 6 membered substituted or unsubstituted, saturated, unsaturated or aromatic, carbocyclic or heterocyclic ring (e.g., piperidine, pyrrolidine, tetrahydrofuran, tetrahydropyran);

R₂₀ is represented by the following structure:

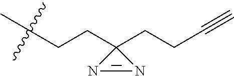 [SMILES: *CCC1(CCC#C)N=N1]

R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

R is H, F, Cl, Br, I, OH, SH, OH, alkoxy, NH(R₁₀), NH—CH₂-cyclopropyl, N(R₁₀)(R₁₁), CF₃, CN, NO₂, COOH, C₁-C₅ linear or branched, substituted or unsubstituted alkyl (e.g., methyl, ethyl, CH₂—OH, CH₂—CH₂—OH, CH₂—CH₂—O—CH₂—CH₂—O—CH₃, CH₂—O—CH₂—CH₂—O—CH₃), C₃-C₈ substituted or unsubstituted cycloalkyl, cyclopropyl, C₁-C₅ linear or branched alkoxy, isopropoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

each R₈ is independently [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂-cyclopropyl, CH₂—CH₂—O—CH₃), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;

or R₁₀ and R₁₁ are joined to form a substituted or unsubstituted 3-8 membered heterocyclic ring (e.g., piperazine, piperidine),

n is an integer between 0 and 4 (e.g., 1, 2);

or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In various embodiments, this invention is directed to a compound represented by the structure of formula I(f):

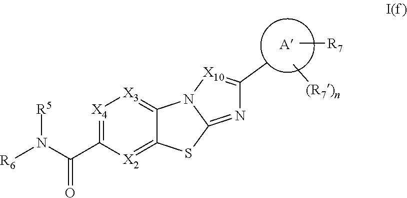

wherein

A′ is a 3-8 membered single or fused, saturated, unsaturated or aromatic carbocyclic or heterocyclic ring (e.g., piperidine, piperazine, isochroman, 1,2,3,4-tetrahydroisoquinoline, indoline, isoindoline, 1,3-dihydroisobenzofuran, 2,3-dihydro-1H-indene, 1,2,3,4-tetrahydronaphthalene);

X₂, X₃, X₄ are each independently nitrogen or CH;

X₁₀ is N, CH, or C(R) (e.g., C(NH—CH₂-cyclopropyl), C(CH₃), C(cyclopropyl), C(isopropoxy));

R₅ is H or C₁-C₅ linear or branched alkyl (e.g. methyl);

R₆ is H, F, Cl, Br, I, OH, SH, R₈—OH, R₈—SH, —R₈—O—R₁₀ (e.g., CH₂—O—CH₃, (CH₂)₂—O—CH₃ (CH₂)₃—O—CH₃, (CH₂)₂—O—CH(CH₃)₂), R₈—S—R₁₀ (e.g., (CH₂)₃—S—(CH₂)₂CH₃), R₈—NHC(O)—R₁₀, —O—R₈—R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-tetrahydrofurane, CH₂-oxa-azaspirodecane, CH₂-azaspiroheptane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₃—N(CH₂CH₃)₂, (CH₂)₃—N(CH(CH₃)₂)₂, (CH₂)₃-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂—C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclobutyl, cyclohexyl, methoxycyclopropyl, methylcyclobutyl, cyclopropyl, aminomethyl-cyclobutyl, methoxycyclobutyl, 2,3-dihydro-1H-indenol), R₈-(substituted or unsubstituted C₃-C₈ cycloalkyl), substituted or unsubstituted 3-8 membered heterocyclic ring (e.g., piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;

or R₆ and R₅ are joined to form a substituted or unsubstituted 5-8 membered heterocyclic ring (e.g., azepane, piperazine, 2-(piperazin-1-yl)acetamide;

or R₆ is represented by the structure of formula B or Bi:

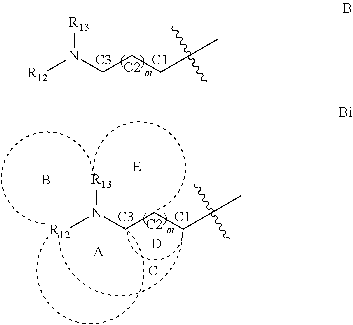

wherein

m is 0 or 1; and
R₁₂ is R₂₀ or C₁-C₅ C(O)-alkyl, and R₁₃ is R₃₀; or
R₁₂ and R₁₃ are both H;
R₁₂ and R₁₃ are each independently H or substituted or unsubstituted C₁-C₅ alkyl (e.g., ethyl, trifluoroethyl);
R₁₂ and C3 are joined to form ring A and R₁₃ is R₃₀; or
R₁₂ and R₁₃ are joined to form ring B; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
   Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
   Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
   Ring D is a substituted or unsubstituted C₃-C₈ cycloalkyl (e.g., cyclobutane, cyclohexane);

R₇ is H, F, Cl, Br, I, OH, O—R₂₀, SH, R₈—OH, R₈—SH, SR₁₀, —R₈—O—R₁₀, —R₈—S—R₁₀, R₈—(C₃-C₈ cycloalkyl), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)), C(O)N(R₁₀)(R₁₁) (e.g., C(O)NH(CH₃), C(O)NH(CH₂CH₂OCH₃), C(O)NH(CH₂CH₂OH)), SO₂R, SO₂N(R₁₀)(R₁₁), CH(CF₃)(NH—R₁₀), C₁-C₅ linear or branched, substituted or unsubstituted alkyl (e.g., methylimidazole, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy, ethoxy), optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclopropanol, cyclohexyl), substituted or unsubstituted 4-7 membered heterocyclic ring (e.g., morpholine, tetrahydrofuran, tetrahydropyran, oxetane, oxetan-3-ol, pyrrolidine, 1-methylpyrrolidine, pyrrolidin-2-one, pyrrolidinone, imidazole, pyrazole, piperazine, piperidine, piperidine-4-carbonitrile, 4-fluoropiperidine, oxadiazole, triazole, 2-oxopyrrolidine), R₈-(substituted or unsubstituted single, fused or spiro 3-8 membered heterocyclic ring), substituted or unsubstituted aryl, substituted or unsubstituted benzyl;

or R₇ is represented by the structure of formula A:

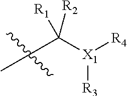

wherein
X₁ is N or O;
R₁ and R₂ are each independently H, F, or CF₃; or
R₁ and R₂ are joined to form ═O or a C₃-C₈ carbocyclic or heterocyclic ring (e.g., cyclopropyl);
R₃ and R₄ are each independently H, Me, substituted or unsubstituted C₁-C₅ alkyl (e.g., methoxyethylene, methylaminoethyl, aminoethyl), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀
R₃ and R₄ are joined to form a 3-8 membered heterocyclic ring (e.g., pyrrolidine, 2-oxopyrrolidine, piperidine, morpholine, piperazine, imidazole);
wherein if X₁ is O then R₄ is absent;

R₇′ is H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted 3-8 membered heterocyclic ring (e.g., morpholine, pyran, oxetane, pyrrolidine, imidazole, piperazine, piperidine, diaoxazole, 2-oxopyrrolidine), substituted or unsubstituted aryl, substituted or unsubstituted benzyl;
or R₇ and R₇′ are joined to form a 5 or 6 membered substituted or unsubstituted, saturated, unsaturated or aromatic, carbocyclic or heterocyclic ring (e.g., piperidine, pyrrolidine, tetrahydrofuran, tetrahydropyran);

R₂₀ is represented by the following structure:

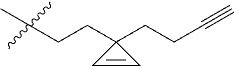 [SMILES: *CCC1(CCC#C)C=C1]

R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

R is H, F, Cl, Br, I, OH, SH, OH, alkoxy, NH(R₁₀), NH—CH₂-cyclopropyl, N(R₁₀)(R₁₁), CF₃, CN, NO₂, COOH, C₁-C₅ linear or branched, substituted or unsubstituted alkyl (e.g., methyl, ethyl, CH₂—OH, CH₂—CH₂—OH, CH₂—CH₂—O—CH₂—CH₂—O—CH₃, CH₂—O—CH₂—CH₂—O—CH₃), C₃-C₈ substituted or unsubstituted cycloalkyl, cyclopropyl, C₁-C₅ linear or branched alkoxy, isopropoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

each R₈ is independently [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂-cyclopropyl, CH₂—CH₂—O—CH₃), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;

or R₁₀ and R₁₁ are joined to form a substituted or unsubstituted 3-8 membered heterocyclic ring (e.g., piperazine, piperidine),

n is an integer between 0 and 4 (e.g., 1, 2);

or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In various embodiments, this invention is directed to a compound represented by the structure of formula I(g):

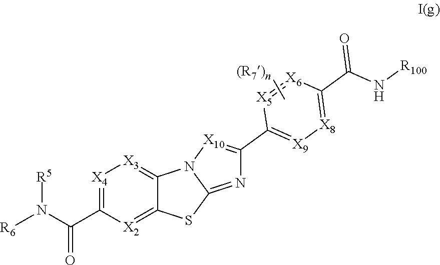

wherein

X₂, X₃, and X₄, are each independently nitrogen or CH;

X₅, X₆, X₇, X₈ and X₉ are each independently nitrogen or carbon atoms;

X₁₀ is N, CH, or C(R) (e.g., C(NH—CH₂-cyclopropyl), C(CH₃), C(cyclopropyl), C(isopropoxy));

R₁₀₀ is a C₁-C₅ linear or branched, substituted or unsubstituted alkyl (e.g., methyl), R₈—OH (e.g., (CH₂)₂—OH), —R₈—O—R₁₀ (e.g., (CH₂)₂—O—CH₃), R₈—N(R₁₀)(R₁₁) (e.g., (e.g., (CH₂)₂-NH(CH₃), (CH₂)₂—NH₂), R₂₀, or a substituted or unsubstituted 3-8 membered heterocyclic ring (e.g., pyrrolidine, piperidine);

R₅ is H or C₁-C₅ linear or branched alkyl (e.g. methyl);

R₆ is H, F, Cl, Br, I, OH, SH, R₈—OH, R₈—SH, —R₈—O—R₁₀ (e.g., CH₂—O—CH₃, (CH₂)₂—O—CH₃ (CH₂)₃—O—CH₃, (CH₂)₂—O—CH(CH₃)₂), R₈—S—R₁₀ (e.g., (CH₂)₃—S—(CH₂)₂CH₃), R₈—NHC(O)—R₁₀, —O—R₈—R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted 3 to 10 membered single, fused or spiro heterocyclic ring) (e.g., (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-tetrahydrofurane, CH₂-oxa-azaspirodecane, CH₂-azaspiroheptane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₃—N(CH₂CH₃)₂, (CH₂)₃—N(CH(CH₃)₂)₂, (CH₂)₃-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₈—C(O)N(R₁₀)(R₁₁) (e.g., (CH₂)₂-C(O)-piperidine), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂-OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclobutyl, cyclohexyl, methoxycyclopropyl, methylcyclobutyl, cyclopropyl, aminomethyl-cyclobutyl, methoxycyclobutyl, 2,3-dihydro-1H-indenol), R₈-(substituted or unsubstituted C₃-C₈ cycloalkyl), substituted or unsubstituted 3-8 membered heterocyclic ring (e.g., piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;

or R₆ and R₅ are joined to for a substituted or unsubstituted 5-8 membered heterocyclic ring (e.g., azepane, piperazine, 2-(piperazin-1-yl)acetamide;

or R₆ is represented by the structure of formula C:

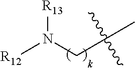

wherein
k is between 1 and 4;
R₁₂ and R₁₃ are each independently H, C₁-C₅ linear or branched, substituted or unsubstituted alkyl (e.g., ethyl, isopropyl), R₂₀, or
R₁₂ and R₁₃ are joined to form a substituted or unsubstituted 4-7 membered heterocyclic ring (e.g., piperidine, piperazine, pyrrolidine, oxa-6-azaspiro[3.3]heptane);

or R₆ is represented by the structure of formula Bi:

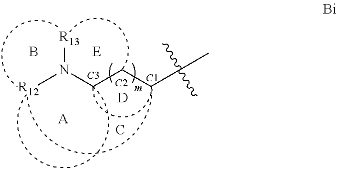

wherein

m is 0 or 1; and
R₁₂ is R₂₀ or C₁-C₅ C(O)-alkyl, and R₁₃ is R₃₀; or
R₁₂ and R₁₃ are both H;
R₁₂ and R₁₃ are each independently H or substituted or unsubstituted C₁-C₅ alkyl (e.g., ethyl, trifluoroethyl);
R₁₂ and C3 are joined to form ring A and R₁₃ is R₃₀; or
R₁₂ and R₁₃ are joined to form ring B; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
   Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
   Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
   Ring D is a substituted or unsubstituted C₃-C₈ cycloalkyl (e.g., cyclobutane, cyclohexane);

R₇′ is H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted 3-8 membered heterocyclic ring (e.g., morpholine, pyran, oxetane, pyrrolidine, imidazole, piperazine, piperidine, diaoxazole, 2-oxopyrrolidine), substituted or unsubstituted aryl, substituted or unsubstituted benzyl;

R₂₀ is represented by the following structure:

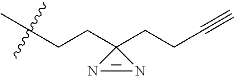 [SMILES: *CCC1(CCC#C)N=N1]

R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

R is H, F, Cl, Br, I, OH, SH, OH, alkoxy, NH(R₁₀), NH—CH₂-cyclopropyl, N(R₁₀)(R₁₁), CF₃, CN, NO₂, COOH, C₁-C₅ linear or branched, substituted or unsubstituted alkyl (e.g., methyl, ethyl, CH₂—OH, CH₂—CH₂—OH, CH₂—CH₂—O—CH₂—CH₂—O—CH₃, CH₂—O—CH₂—CH₂—O—CH₃), C₃-C₈ substituted or unsubstituted cycloalkyl, cyclopropyl, C₁-C₅ linear or branched alkoxy, isopropoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

each R₈ is independently [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂-cyclopropyl, CH₂—CH₂—O—CH₃), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;

or R₁₀ and R₁₁ are joined to form a substituted or unsubstituted 3-8 membered heterocyclic ring (e.g., piperazine, piperidine),

n is an integer between 0 and 4 (e.g., 1, 2);

or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In some embodiments, if R₁₀₀ is methyl and R₅ is H, then R₁₂ and R₁₃ are not both alkyls. In some embodiments, if R₁₀₀ is methyl and R₅ is H, then R₁₂ and R₁₃ cannot be joined to form piperidine.

In various embodiments, this invention is directed to a compound represented by the structure of formula I(h):

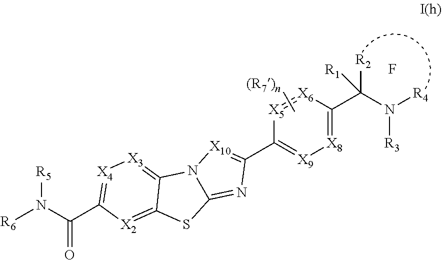

wherein
Ring F is absent or is a substituted or unsubstituted, saturated or unsaturated, 4-8 membered heterocyclic ring (e.g., pyrrolidine, 1-methylpyrrolidine, pyrrolidin-2-one, pyridine, piperidine, imidazole, pyrimidine, triazole, oxadiazole, pyrazole);
R₁ and R₂ are each independently H, F, Cl, Br, I, OH, SH, or CF₃, substituted or unsubstituted C₁-C₅ alkyl, C₁-C₅ linear or branched, or C₃-C₈ cyclic haloalkyl, substituted or unsubstituted C₁-C₅ linear or branched, or C₃-C₈ cyclic alkoxy;
or R₁ and R₂ are joined to form a 3-8 membered carbocyclic or heterocyclic ring (e.g., cyclopropyl);
or R₂ and R₄ are joined to form Ring F as defined above (e.g., pyrrolidine, pyridine, pyrimidine, triazole, oxadiazole, pyrazole), wherein if Ring F is aromatic, then R₁ and/or R₃ are absent;
R₃ and R₄ are each independently H, Me, substituted or unsubstituted C₁-C₅ alkyl (e.g., methoxyethylene, methylaminoethyl, aminoethyl), —R₈—O—R₁₀ (e.g., (CH₂)₂—O—CH₃), R₈—N(R₁₀)(R₁₁) (e.g., (CH₂)₂-NH(CH₃)), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀; or
R₃ and R₄ are joined to form a 3-8 membered heterocyclic ring (e.g., pyrrolidine, pyrrolidone, 2-oxopyrrolidine, piperidine, morpholine, piperazine, imidazole);

X₂, X₃, and X₄, are each independently nitrogen or CH;

X₅, X₆, X₇, X₈ and X₉ are each independently nitrogen or carbon atoms;

X₁₀ is N, CH, or C(R) (e.g., C(NH—CH₂-cyclopropyl), C(CH₃), C(cyclopropyl), C(isopropoxy));

R₅ is H or C₁-C₅ linear or branched alkyl (e.g. methyl);

R₆ is H, F, Cl, Br, I, OH, SH, R₈—OH, R₈—SH, —R₈—O—R₁₀ (e.g., CH₂—O—CH₃, (CH₂)₂O—CH₃ (CH₂)₃O—CH₃, (CH₂)₂O—CH(CH₃)₂), R₈—S—R₁₀ (e.g., (CH₂)₃—S—(CH₂)₂CH₃), R₈—NHC(O)—R₁₀, —O—R₈—R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-pyran, (CH₂)₂-pyrrazole, (CH₂)₂-imidazole, CH₂-tetrahydrofurane, CH₂-dioxane, CH₂-oxetane, (CH₂)₃-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-tetrahydrofurane, CH₂-oxa-azaspirodecane, CH₂-azaspiroheptane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₃—N(CH₂CH₃)₂, (CH₂)₃—N(CH(CH₃)₂)₂, (CH₂)₃-piperidine, (CH₂)₃-4-fluoro-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂-C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclobutyl, cyclohexyl, methoxycyclopropyl, methylcyclobutyl, cyclopropyl, aminomethyl-cyclobutyl, methoxycyclobutyl, 2,3-dihydro-1H-indenol), R₈-(substituted or unsubstituted C₃-C₈ cycloalkyl), substituted or unsubstituted 3-8 membered heterocyclic ring (e.g., piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;

or R₆ and R₅ are joined to for a substituted or unsubstituted 5-8 membered heterocyclic ring (e.g., azepane, piperazine, 2-(piperazin-1-yl)acetamide;

or R₆ is represented by the structure of formula B or Bi:

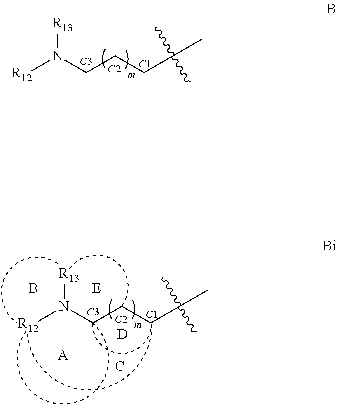

wherein

m is 0 or 1; and
R₁₂ is R₂₀ or C₁-C₅ C(O)-alkyl, and R₁₃ is R₃₀; or
R₁₂ and R₁₃ are both H;
R₁₂ and R₁₃ are each independently H or substituted or unsubstituted C₁-C₅ alkyl (e.g., ethyl, trifluoroethyl);
R₁₂ and C3 are joined to form ring A and R₁₃ is R₃₀; or
R₁₂ and R₁₃ are joined to form ring B; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
   Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
   Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
   Ring D is a substituted or unsubstituted C₃-C₈ cycloalkyl (e.g., cyclobutane, cyclohexane);

R₇′ is H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted 3-8 membered heterocyclic ring (e.g., morpholine, pyran, oxetane, pyrrolidine, imidazole, piperazine, piperidine, diaoxazole, 2-oxopyrrolidine), substituted or unsubstituted aryl, substituted or unsubstituted benzyl;

R₂₀ is represented by the following structure:

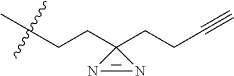 [SMILES: *CCC1(CCC#C)N=N1]

R is H, F, Cl, Br, I, OH, SH, OH, alkoxy, NH(R₁₀), NH—CH₂-cyclopropyl, N(R₁₀)(R₁₁), CF₃, CN, NO₂, COOH, C₁-C₅ linear or branched, substituted or unsubstituted alkyl (e.g., methyl, ethyl, CH₂—OH, CH₂—CH₂—OH, CH₂—CH₂—O—CH₂—CH₂—O—CH₃, CH₂—O—CH₂—CH₂—O—CH₃), C₃-C₈ substituted or unsubstituted cycloalkyl, cyclopropyl, C₁-C₅ linear or branched alkoxy, isopropoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

each R₈ is independently [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂-cyclopropyl, CH₂—CH₂—O—CH₃), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;

or R₁₀ and R₁₁ are joined to form a substituted or unsubstituted 3-8 membered heterocyclic ring (e.g., piperazine, piperidine),

n is an integer between 0 and 4 (e.g., 1, 2);

or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In various embodiments, this invention is directed to a compound represented by the structure of formula I(i):

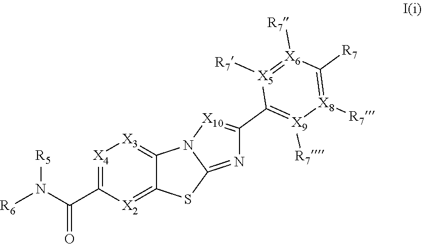

wherein

X₂, X₃, and X₄, are each independently nitrogen or CH;

X₅, X₆, X₇, X₈ and X₉ are each independently nitrogen or carbon atoms;

X₁₀ is N, CH, or C(R) (e.g., C(NH—CH₂-cyclopropyl), C(CH₃), C(cyclopropyl), C(isopropoxy));

R₅ is H or C₁-C₅ linear or branched alkyl (e.g. methyl);

R₆ is H, F, Cl, Br, I, OH, SH, R₈—OH, R₈—SH, —R₈—O—R₁₀ (e.g., CH₂—O—CH₃, (CH₂)₂—O—CH₃ (CH₂)₃—O—CH₃, (CH₂)₂—O—CH(CH₃)₂), R₈—S—R₁₀ (e.g., (CH₂)₃—S—(CH₂)₂CH₃), R₈—NHC(O)—R₁₀, —O—R₈—R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-pyran, (CH₂)₂-pyrrazole, (CH₂)₂-imidazole, CH₂-tetrahydrofurane, CH₂-dioxane, CH₂-oxetane, (CH₂)₃-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-tetrahydrofurane, CH₂-oxa-azaspirodecane, CH₂-azaspiroheptane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₃—N(CH₂CH₃)₂, (CH₂)₃—N(CH(CH₃)₂)₂, (CH₂)₃-piperidine, (CH₂)₃-4-fluoro-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂-C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclobutyl, cyclohexyl, methoxycyclopropyl, methylcyclobutyl, cyclopropyl, aminomethyl-cyclobutyl, methoxycyclobutyl, 2,3-dihydro-1H-indenol), R₈-(substituted or unsubstituted C₃-C₈ cycloalkyl), substituted or unsubstituted 3-8 membered heterocyclic ring (e.g., piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;

or R₆ and R₅ are joined to for a substituted or unsubstituted 5-8 membered heterocyclic ring (e.g., azepane, piperazine, 2-(piperazin-1-yl)acetamide;

or R₆ is represented by the structure of formula B or Bi:

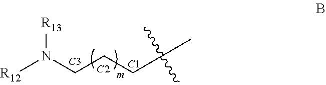

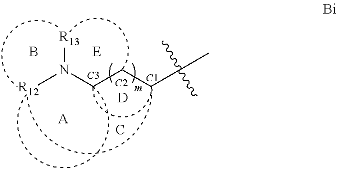

wherein

m is 0 or 1; and
R₁₂ is R₂₀ or C₁-C₅ C(O)-alkyl, and R₁₃ is R₃₀; or
R₁₂ and R₁₃ are both H;
R₁₂ and R₁₃ are each independently H or substituted or unsubstituted C₁-C₅ alkyl (e.g., ethyl, trifluoroethyl);
R₁₂ and C3 are joined to form ring A and R₁₃ is R₃₀; or
R₁₂ and R₁₃ are joined to form ring B; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
   Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
   Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
   Ring D is a substituted or unsubstituted C₃-C₈ cycloalkyl (e.g., cyclobutane, cyclohexane);

R₇, R₇′, R₇″, R₇′″ and R₇″″ are each independently H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted 3-8 membered heterocyclic ring (e.g., morpholine, pyran, oxetane, pyrrolidine, imidazole, piperazine, piperidine, diaoxazole, 2-oxopyrrolidine), substituted or unsubstituted aryl, substituted or unsubstituted benzyl;

with the proviso that at least two of R₇, R₇′, R₇″, R₇′″ and R₇″″ are not H;

or R₇′ and R₇″ are joined to form a 3-8 membered substituted or unsubstituted, saturated, unsaturated or aromatic, carbocyclic or heterocyclic ring (e.g., cyclopentyl, cyclohexyl, piperidine, tetrahydrofuran, tetrahydropyran, pyrrolidine);

or R₇″ and R₇ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);

or R₇ and R₇′″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);

or R₇′″ and R₇″″ are joined to form a 3-8 membered substituted or unsubstituted carbocyclic or heterocyclic ring (e.g., cyclopentyl, cyclohexyl, piperidine, tetrahydrofuran, tetrahydropyran, pyrrolidine);

R₂₀ is represented by the following structure:

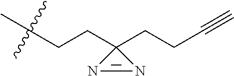 [SMILES: *CCC1(CCC#C)N=N1]

R is H, F, Cl, Br, I, OH, SH, OH, alkoxy, NH(R₁₀), NH—CH₂-cyclopropyl, N(R₁₀)(R₁₁), CF₃, CN, NO₂, COOH, C₁-C₅ linear or branched, substituted or unsubstituted alkyl (e.g., methyl, ethyl, CH₂—OH, CH₂—CH₂—OH, CH₂—CH₂—O—CH₂—CH₂—O—CH₃, CH₂—O—CH₂—CH₂—O—CH₃), C₃-C₈ substituted or unsubstituted cycloalkyl, cyclopropyl, C₁-C₅ linear or branched alkoxy, isopropoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

each R₈ is independently [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂-cyclopropyl, CH₂—CH₂—O—CH₃), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;

or R₁₀ and R₁₁ are joined to form a substituted or unsubstituted 3-8 membered heterocyclic ring (e.g., piperazine, piperidine),

n is an integer between 0 and 4 (e.g., 1, 2);

or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In various embodiments, this invention is directed to a compound represented by the structure of formula (II):

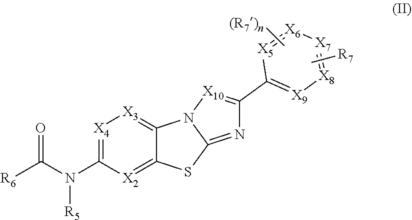

wherein

X₂, X₃, and X₄, are each independently nitrogen or CH;

X₅, X₆, X₇, X₈ and X₉ are each independently nitrogen or carbon atoms;

X₁₀ is N, CH, or C(R) (e.g., C(NH—CH₂-cyclopropyl), C(CH₃), C(cyclopropyl), C(isopropoxy));

R₅ is H or C₁-C₅ linear or branched alkyl (e.g. methyl);

R₆ is H, F, Cl, Br, I, OH, SH, R₈—OH, R₈—SH, —R₈—O—R₁₀ (e.g., CH₂—O—CH₃, (CH₂)₂O—CH₃ (CH₂)₃O—CH₃, (CH₂)₂O—CH(CH₃)₂), R₈—S—R₁₀ (e.g., (CH₂)₃—S—(CH₂)₂CH₃), R₈—NHC(O)—R₁₀, —O—R₈—R₁₀, R₈-(substituted or unsubstituted C₃-C₈ cycloalkyl) (e.g., CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted, saturated, unsaturated or aromatic, single, fused or spiro 3-10 membered heterocyclic ring) (e.g., (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-tetrahydrofurane, CH₂-oxa-azaspirodecane, CH₂-azaspiroheptane, (CH₂)₃-dimethylpyrazole, CH₂-2-oxo-methylpyrrolidine, CH₂-methyl-azetidine, CH₂-azaspiroheptane), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁) (e.g., (CH₂)₃—N(CH₂CH₃)₂, (CH₂)₃—N(CH(CH₃)₂)₂, (CH₂)₃-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃)), R₉—R₈—N(R₁₀)(R₁₁) (e.g., (CH₂)₂—C(O)-piperidine), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl, cyclobutyl, cyclohexyl, methoxycyclopropyl, methylcyclobutyl, cyclopropyl, aminomethyl-cyclobutyl, methoxycyclobutyl, 2,3-dihydro-1H-indenol), R₈-(substituted or unsubstituted C₃-C₈ cycloalkyl), substituted or unsubstituted 3-8 membered heterocyclic ring (e.g., piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), substituted or unsubstituted benzyl;

or R₆ and R₅ are joined to for a substituted or unsubstituted 5-8 membered heterocyclic ring (e.g., azepane, piperazine, 2-(piperazin-1-yl)acetamide;

or R₆ is represented by the structure of formula B or Bi:

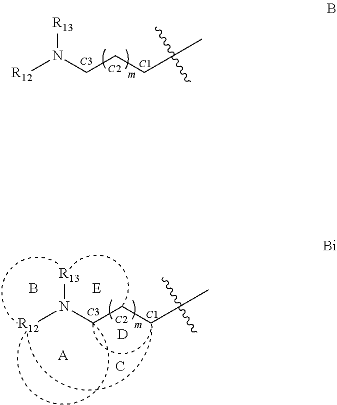

wherein

m is 0 or 1; and
R₁₂ is R₂₀ or C₁-C₅ C(O)-alkyl, and R₁₃ is R₃₀; or
R₁₂ and R₁₃ are both H;
R₁₂ and R₁₃ are each independently H or substituted or unsubstituted C₁-C₅ alkyl (e.g., ethyl, trifluoroethyl);
R₁₂ and C3 are joined to form ring A and R₁₃ is R₃₀; or
R₁₂ and R₁₃ are joined to form ring B; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
   Ring A, C and E are each independently a substituted or unsubstituted single spiro or fused 3-8 membered heterocyclic ring (e.g., A: pyrrolidine, methylpyrrolidine, ethylpyrrolidine); C: piperidine, pyrrolidine, methyl-2-oxopyrrolidine, pyran-pyrrolidine, methyl-azetidine, azabicyclooctane, 2-azabicyclo[2.1.1]hexane, 2-azaspiro[3.3]heptane; E: pyrrolidine, azetidine, ethylpyrrolidine, oxopyrrolidine, methylpiperidine);
   Ring B is a substituted or unsubstituted single, spiro or fused 3-8 membered heterocyclic ring (B: piperidine, piperidin-2-one, 4-fluoropiperidin-2-one, piperidine-4-carbonitrile, 4-fluoropiperidine, 4-fluoro-2-methylpiperidine, methyl-piperidin, fluoropiperidine, difluoropiperidine, pyrrolidine, piperazine, methylpyrrolidine, thiomorpholine 1,1-dioxide, 2-oxa-6-azaspiro[3.3]heptane, methyl-piperazine, dimethyl-pyrazole, imidazole, 2-methyl-2,5-diazabicyclo[2.2.1]heptane, hydroxymethyl-pyrrolidine, diazabicyclo[2.2.1]heptane, 6-fluoro-3-azabicyclo[3.1.1]heptane; and
   Ring D is a substituted or unsubstituted C₃-C₈ cycloalkyl (e.g., cyclobutane, cyclohexane);

R₇ is H, F, Cl, Br, I, OH, O—R₂₀, SH, R₈—OH, R₈—SH, SR₁₀, —R₈—O—R₁₀, —R₈—S—R₁₀, R₈—(C₃-C₈ cycloalkyl), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR (e.g., C(O)NH(CH₃)), C(O)N(R₁₀)(R₁₁) (e.g., C(O)NH(CH₃), C(O)NH(CH₂CH₂OCH₃), C(O)NH(CH₂CH₂OH)), SO₂R, SO₂N(R₁₀)(R₁₁), CH(CF₃)(NH—R₁₀), C₁-C₅ linear or branched, substituted or unsubstituted alkyl (e.g., methylimidazole, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy, ethoxy), optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropanol, cyclohexyl), substituted or unsubstituted 4-7 membered heterocyclic ring (e.g., morpholine, tetrahydrofuran, tetrahydropyran, oxetane, oxetan-3-ol, pyrrolidine, 1-methylpyrrolidine, pyrrolidin-2-one, pyrrolidinone, imidazole, pyrazole, piperazine, piperidine, piperidine-4-carbonitrile, 4-fluoropiperidine, oxadiazole, triazole, 2-oxopyrrolidine), R₈-(substituted or unsubstituted single, fused or spiro 3-8 membered heterocyclic ring), substituted or unsubstituted aryl, substituted or unsubstituted benzyl;

or R₇ is represented by the structure of formula A:

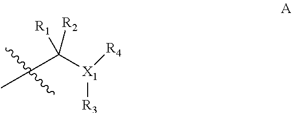

wherein
X₁ is N or O;
R₁ and R₂ are each independently H, F, or CF₃; or
R₁ and R₂ are joined to form ═O or a C₃-C₈ carbocyclic or heterocyclic ring (e.g., cyclopropyl);
R₃ and R₄ are each independently H, Me, substituted or unsubstituted C₁-C₅ alkyl (e.g., methoxyethylene, methylaminoethyl, aminoethyl), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀
R₃ and R₄ are joined to form a 3-8 membered heterocyclic ring (e.g., pyrrolidine, 2-oxopyrrolidine, piperidine, morpholine, piperazine, imidazole);
wherein if X₁ is O then R₄ is absent;

R₇′ is H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted 3-8 membered heterocyclic ring (e.g., morpholine, pyran, oxetane, pyrrolidine, imidazole, piperazine, piperidine, diaoxazole, 2-oxopyrrolidine), substituted or unsubstituted aryl, substituted or unsubstituted benzyl;
or R₇ and R₇′ are joined to form a 5 or 6 membered substituted or unsubstituted, saturated, unsaturated or aromatic, carbocyclic or heterocyclic ring (e.g., piperidine, pyrrolidine, tetrahydrofuran, tetrahydropyran);

R₂₀ is represented by the following structure:

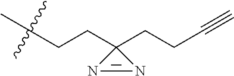 [SMILES: *CCC1(CCC#C)N=N1]

R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

R is H, F, Cl, Br, I, OH, SH, OH, alkoxy, NH(R₁₀), NH—CH₂-cyclopropyl, N(R₁₀)(R₁₁), CF₃, CN, NO₂, COOH, C₁-C₅ linear or branched, substituted or unsubstituted alkyl (e.g., methyl, ethyl, CH₂—OH, CH₂—CH₂—OH, CH₂—CH₂—O—CH₂—CH₂—O—CH₃, CH₂—O—CH₂—CH₂—O—CH₃), C₃-C₈ substituted or unsubstituted cycloalkyl, cyclopropyl, C₁-C₅ linear or branched alkoxy, isopropoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);

each R₈ is independently [CH₂]ₚ
wherein p is between 1 and 10;

R₉ is [CH]_{q}, [C]_{q}
wherein q is between 2 and 10;

R₁₀ and R₁₁ are each independently H, C₁-C₅ substituted or unsubstituted linear or branched alkyl (e.g., methyl, ethyl, CH₂-cyclopropyl, CH₂—CH₂—O—CH₃), C₁-C₅ substituted or unsubstituted linear or branched haloalky (e.g., CH₂CF₃), C₁-C₅ linear or branched alkoxy (e.g., O—CH₃), R₂₀, C(O)R, or S(O)₂R;

or R₁₀ and R₁₁ are joined to form a substituted or unsubstituted 3-8 membered heterocyclic ring (e.g., piperazine, piperidine),

n is an integer between 0 and 4 (e.g., 1, 2);

or its pharmaceutically acceptable salt, stereoisomer, tautomer, hydrate, N-oxide, reverse amide analog, prodrug, isotopic variant (e.g., deuterated analog), PROTAC, pharmaceutical product or any combination thereof.

In some embodiments, X₂ of formula I, II and/or I(a)-I(i) is a nitrogen atom. In other embodiments, X₂ is a CH.

In some embodiments, X₃ of formula I, II and/or I(a)-I(i) is a nitrogen atom. In other embodiments, X₃ is a CH.

In some embodiments, X₄ of formula I, II and/or I(a)-I(i) is a nitrogen atom. In other embodiments, X₄ is a CH.

In some embodiments, X₅ of formula I, II and/or I(a)-I(i) is a nitrogen atom. In other embodiments, X₅ is a carbon atom.

In some embodiments, X₆ of formula I, II and/or I(a)-I(i) is a nitrogen atom. In other embodiments, X₆ is a carbon atom.

In some embodiments, X₇ of formula I, II and/or I(a)-I(i) is a nitrogen atom. In other embodiments, X₇ is a carbon atom.

In some embodiments, X₈ of formula I, II and/or I(a)-I(i) is a nitrogen atom. In other embodiments, X₈ is a carbon atom.

In some embodiments, X₉ of formula I, II and/or I(a)-I(i) is a nitrogen atom. In other embodiments, X₉ is a carbon atom.

In some embodiments, X₁₀ of formula I, II and/or I(a)-I(i) is a nitrogen atom. In other embodiments, X₁₀ is N. In other embodiments, X₁₀ is CH. In other embodiments, X₁₀ is C(R), wherein R is as defined below. In other embodiments, X₁₀ is C(R), wherein R is an alkyl. In other embodiments, X₁₀ is C(R), wherein R is a methyl. In other embodiments, X₁₀ is C(R), wherein R is a cycloalkyl. In other embodiments, X₁₀ is C(R), wherein R is a cyclopropyl. In other embodiments, X₁₀ is C(R), wherein R is a COOH. In other embodiments, X₁₀ is C(R), wherein R is N(H)R₁₀; and R₁₀ is a substituted alkyl. In other embodiments, X₁₀ is C(N(H)(CH₂-cyclopropyl)). In other embodiments, X₁₀ is C(R), wherein R is a substituted alkyl. In other embodiments, X₁₀ is C(R), wherein R is CH₂—OH. In other embodiments, X₁₀ is C(R), wherein R is CH₂—CH₂—OH. In other embodiments, X₁₀ is C(R), wherein R is an alkoxy. In other embodiments, X₁₀ is C(R), wherein R is a isopropoxy.

In some embodiments, at least one of X₂, X₃, X₄, X₅, X₆, X₇, X₈ and X₉ of formula I, II and/or I(a)-I(i) is a nitrogen atom. In some embodiments, at least one of X₂, X₃, X₄, X₅, X₆, X₇, X₈ and X₉ of formula I(d) is a nitrogen atom. In some embodiments, at least one of X₂, X₃, X₄, X₅, X₆, X₇, X₈, X₉ and X₁₀ of formula I(d) is a nitrogen atom.

In some embodiments, R₅ of formula I, II and/or I(a)-I(i) is H. In other embodiments, R₅ is C₁-C₅ linear or branched alkyl. In other embodiments, R₅ is methyl. In other embodiments, R₅ is methyl, ethyl, propyl, isopropyl, butyl, t-butyl, iso-butyl, pentyl, neopentyl; each represents a separate embodiment according to this invention.

In some embodiments, R₅ and R₆ of formula I, II and/or I(a)-I(i) are joined to form a substituted or unsubstituted 5-8 membered heterocyclic ring. In some embodiments, R₅ and R₆ are joined to form a substituted 5-8 membered heterocyclic ring. In some embodiments, R₅ and R₆ are joined to form an unsubstituted 5-8 membered heterocyclic ring. In some embodiments, the heterocyclic ring is azepane, piperazine or 2-(piperazin-1-yl)acetamide; each represents a separate embodiment according to this invention. In some embodiments, the heterocyclic ring is substituted with at least one substitution selected from: F, Cl, Br, I, CF₃, R₂₀, C₁-C₅ linear or branched alkyl, C₁-C₅ linear or branched haloalkyl, OH, alkoxy, R₈—OH (e.g., CH₂—OH), OMe, amide, C(O)N(R)₂, C(O)N(R₁₀)(R₁₁), R₈—C(O)N(R₁₀)(R₁₁), C(O)-pyrrolidine, C(O)-piperidine, N(R)₂, NH(R₁₀), N(R₁₀)(R₁₁), N(CH₃)₂, NH₂, CF₃, aryl, phenyl, heteroaryl, substituted or unsubstituted C₃-C₈ cycloalkyl, cyclobutanol, substituted or unsubstituted 3-8 membered heterocyclic ring, which may be saturated, unsaturated, aromatice, single fused or spiral, pyran, oxetane, piperidine, pyrazole, methyl-pyrrazole, triazole, imidazole, halophenyl, (benzyloxy)phenyl, CN, and NO₂; each is a separate embodiment according to this invention. In some embodiments, the heterocyclic ring of formula I(e) is not substituted with CO₂—R.

In some embodiments, R₆ of formula I, II and/or I(a)-I(i) is H. In other embodiments, R₆ is H, F, Cl, Br, I, OH, SH, R₈—OH, R₈—SH, —R₈—O—R₁₀, CH₂—O—CH₃, (CH₂)₂—O—CH₃ (CH₂)₃—O—CH₃, (CH₂)₂—O—CH(CH₃)₂, R₈—S—R₁₀, (CH₂)₃—S—(CH₂)₂CH₃, R₈—NHC(O)—R₁₀, —O—R₈—R₁₀, R₈-(substituted or unsubstituted C₃-C₈ cycloalkyl), CH₂-cyclopropyl, CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), R₈-(substituted or unsubstituted single, fused or spiro 3-8 membered heterocyclic ring), (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂—methyl-THF, CH₂-ethyl-piperidine, CH₂-tetrahydrofurane, CH₂-oxa-azaspirodecane, CH₂-azaspiroheptane, (CH₂)₃-dimethylpyrazole, CH₂-2-oxo-methylpyrrolidine, CH₂-methyl-azetidine, CH₂-azaspiroheptane, CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), (CH₂)₃—N(CH₂CH₃)₂, (CH₂)₃—N(CH(CH₃)₂)₂, (CH₂)₃-piperidine, (CH₂)₃-4-fluoro- piperidine, (CH₂)₃-piperidine-2-one, (CH₂)₃-4-cyano-piperidine, (CH₂)₃-4-trifluoromethyl-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, (CH₂)₃—N(CH₂CH₃)(CH₂CF₃), R₈—C(O)N(R₁₀)(R₁₁), (CH₂)₂-C(O)-piperidine, R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃, CH(CH₃)C(O)N(CH₃)₂, C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy methoxy, optionally wherein at least one methylene group (CH₂) in the alkoxy is replaced with an oxygen atom, O—(CH₂)₂O—CH₃, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, cyclopropyl, cyclobutyl, cyclohexyl, methoxycyclopropyl, methylcyclobutyl, cyclopropyl, aminomethyl-cyclobutyl, methoxycyclobutyl, 2,3-dihydro-1H-indenol, R₈-(substituted or unsubstituted C₃-C₈ cycloalkyl), substituted or unsubstituted 3-8 membered heterocyclic ring (e.g., piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane), substituted or unsubstituted aryl, substituted or unsubstituted R₈-aryl (e.g., benzyl), or substituted or unsubstituted benzyl; each represents a separate embodiment according to this invention. In some embodiments, R₆ may be further substituted with at least one substitution selected from: F, Cl, Br, I, C₁-C₅ linear or branched alkyl, OH, alkoxy, OMe, amide, C(O)N(R)₂, C(O)-alkyl, C(O)-pyrrolidine, C(O)-piperidine, N(R)₂, NH(R₁₀), N(R₁₀)(R₁₁), N(CH₃)₂, NH₂, CF₃, aryl, phenyl, heteroaryl, substituted or unsubstituted C₃-C₈ cycloalkyl, cyclobutanol, substituted or unsubstituted 3-8 membered heterocyclic ring pyran, oxetane, piperidine, pyrazole, methyl-pyrrazole, triazole, imidazole, halophenyl, (benzyloxy)phenyl, CN, and NO₂; each represents a separate embodiment according to this invention. In some embodiments, R₆ is H. In some embodiments, R₆ is —R₈—O—R₁₀. In some embodiments, R₆ is CH₂—O—CH₃. In some embodiments, R₆ is R₈—S—R₁₀. In some embodiments, R₆ is (CH₂)₃—S—(CH₂)₂CH₃. In some embodiments, R₆ is R₈—NHC(O)—R₁₀. In some embodiments, R₆ is (CH₂)₃—NHC(O)—R₁₀. In some embodiments, R₆ is (CH₂)—NHC(O)—R₁₀. In some embodiments, R₆ is R₈-(substituted or unsubstituted C₃-C₈ cycloalkyl). Examples of R₈-(substituted or unsubstituted C₃-C₈ cycloalkyl) include but not limited to: CH₂-cyclobutanol, CH₂-difluorocyclopropyl, CH₂-methylcyclopropyl, CH₂-dimethylamino-cyclohexyl, (CH₂)₂-cyclopentanole, and CH₂-cyclohexanol; each represents a separate embodiment according to this invention. In some embodiments, R₆ is R₈-(substituted or unsubstituted saturated, unsaturated or aromatic, single, fused or spiro 3-8 membered heterocyclic ring). In some embodiments, R₆ is R₈-(substituted or unsubstituted saturated, single 3-8 membered heterocyclic ring). In some embodiments, R₆ is R₈-(substituted or unsubstituted unsaturated, single 3-8 membered heterocyclic ring). In some embodiments, R₆ is R₈-(substituted or unsubstituted aromatic, single 3-8 membered heterocyclic ring). In some embodiments, R₆ is R₈-(substituted or unsubstituted saturated, fused 3-8 membered heterocyclic ring). In some embodiments, R₆ is R₈-(substituted or unsubstituted unsaturated, fused 3-8 membered heterocyclic ring). In some embodiments, R₆ is R₈-(substituted or unsubstituted aromatic, fused 3-8 membered heterocyclic ring). In some embodiments, R₆ is R₈-(substituted or unsubstituted spiro 3-8 membered heterocyclic ring). Examples of R₈-(substituted or unsubstituted saturated, unsaturated or aromatic, single, fused or spiro 3-8 membered heterocyclic ring) include but not limited to: (CH₂)₃-pyran, (CH₂)₂-pyrrazole, (CH₂)₂-imidazole, CH₂-tetrahydrofurane, CH₂-dioxane, CH₂-oxetane, CH₂-piperidine, CH₂-triazole, CH₂-1-oxa-8-azaspiro[4.5]decane, (CH₂)₃-diazabicyclo[2.2.1]heptane, CH₂-methyl-THF, CH₂-ethyl-piperidine, CH₂-tetrahydrofurane, CH₂-oxa-azaspirodecane, CH₂-azaspiroheptane, (CH₂)₃-dimethylpyrazole, CH₂-2-oxo-methylpyrrolidine, CH₂-methyl-azetidine, and CH₂-azaspiroheptane. In some embodiments, R₆ is NH₂. In some embodiments, R₆ is NHR. In some embodiments, R₆ is N(R)₂. In some embodiments, R₆ is NH(R₁₀). In some embodiments, R₆ is N(R₁₀)(R₁₁). In some embodiments, R₆ is R₈—N(R₁₀)(R₁₁). In some embodiments, R₈—N(R₁₀)(R₁₁) includes but not limited to: (CH₂)₃—N(CH₂CH₃)₂, (CH₂)₃—N(CH(CH₃)₂)₂, (CH₂)₃-piperidine, (CH₂)₄—NH(CH₃), (CH₂)₃—NH—CH₃, (CH₂)₃—NH—CH₂CH₃, (CH₂)₃—N(CH₂CH₃)₂, (CH₂)₃—NH₂, and (CH₂)₃—N(CH₂CH₃)(CH₂CF₃). In some embodiments, R₆ is R₈—C(O)N(R₁₀)(R₁₁) such as (CH₂)₂-C(O)-piperidine. In some embodiments, R₆ is C₁-C₅ linear or branched, substituted or unsubstituted alkyl. Examples of C₁-C₅ linear or branched, substituted or unsubstituted alkyl include but not limited to: CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), CH(CH₃)C(O)N(CH₃)₂, benzyl, methyl, ethyl, and CH₂—OCH₂—CH₂—O—CH₃. In some embodiments, R₆ is substituted or unsubstituted C₃-C₈ cycloalkyl. In some embodiments, substituted or unsubstituted C₃-C₈ cycloalkyl include: cyclopropyl, cyclobutyl, cyclohexyl, methoxycyclopropyl, methylcyclobutyl, cyclopropyl, aminomethyl-cyclobutyl, methoxycyclobutyl and 2,3-dihydro-1H-indeno. In some embodiments, R₆ is R₈-(substituted or unsubstituted C₃-C₈ cycloalkyl). In some embodiments, R₆ is substituted or unsubstituted saturated, unsaturated or aromatic, single, fused or spiro 3-10 membered heterocyclic ring. In some embodiments, the substituted or unsubstituted saturated, unsaturated or aromatic, single, fused or spiro 3-10 membered heterocyclic ring is piperidine, azetidine, pyrrolidine, pyrrolidinone, quinuclidine, tetrahydropyran, azaspiro[3.3]heptane, imidazole, trifluoromethyl-oxetane, hydroxy-tetrahydrofurane, azepan-2-one, azabicyclohexane; each represents a separate embodiment according to this invention. In some embodiments, R₆ is substituted or unsubstituted R₈-aryl, such as benzyl. In some embodiments, R₆ may be further substituted by at least one substitution selected from: F, Cl, Br, I, CF₃, R₂₀, C₁-C₅ linear or branched alkyl, C₁-C₅ linear or branched haloalkyl, OH, alkoxy, R₈—OH (e.g., CH₂—OH), OMe, amide, C(O)N(R)₂, C(O)N(R₁₀)(R₁₁), R₈—C(O)N(R₁₀)(R₁₁), C(O)-pyrrolidine, C(O)-piperidine, N(R)₂, NH(R₁₀), N(R₁₀)(R₁₁), N(CH₃)₂, NH₂, CF₃, aryl, phenyl, heteroaryl, substituted or unsubstituted C₃-C₈ cycloalkyl, cyclobutanol, substituted or unsubstituted 3-8 membered heterocyclic ring, which may be saturated, unsaturated, aromatice, single fused or spiral, pyran, oxetane, piperidine, pyrazole, methyl-pyrrazole, triazole, imidazole, halophenyl, (benzyloxy)phenyl, CN, and NO₂; each is a separate embodiment according to this invention.

In some embodiments, R₆ and R % of formula I, II and/or I(a)-I(i) are joined to form a substituted or unsubstituted saturated, unsaturated or aromatic, single, fused or spiro 5-8 membered heterocyclic ring. In some embodiments, the substituted or unsubstituted saturated, unsaturated or aromatic, single, fused or spiro 5-8 membered heterocyclic ring is azepane, piperazine, or 2-(piperazin-1-yl)acetamide; each represents a separate embodiment according to this invention. In some embodiments, the ring may be further substituted by at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₆ of formula I, II and/or I(a)-I(i) is represented by the structure of formula B:

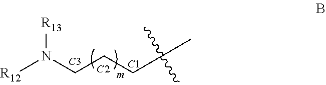

wherein

m is 0 or 1; and
R₁₂ is R₂₀ or C₁-C₅ C(O)-alkyl, and R₁₃ is R₃₀; or
R₁₂ and R₁₃ are both H; or
R₁₂ and R₁₃ are each independently H or substituted or unsubstituted C₁-C₅ alkyl (e.g., ethyl, trifluoroethyl); or
R₁₂ and C3 are joined to form ring A and R₁₃ is R₃₀; or
R₁₂ and R₁₃ are joined to form ring B; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
   Ring A, C and E are each independently a substituted or unsubstituted single spiro or fused 3-8 membered heterocyclic rings;
   Ring B is a substituted or unsubstituted single, spiro or fused 3-8 membered heterocyclic ring; and
   Ring D is a substituted or unsubstituted C₃-C₈ cycloalkyl;

In some embodiments, formula B is represented by formula Bi.

In some embodiments, R₆ of formula I, II and/or I(a)-I(i) is represented by the structure of formula Bi:

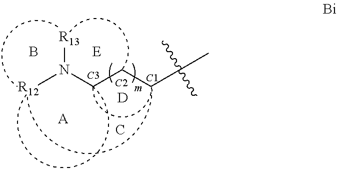

wherein

m is 0 or 1; and
R₁₂ is R₂₀ or C₁-C₅ C(O)-alkyl, and R₁₃ is R₃₀; or
R₁₂ and R₁₃ are both H; or
R₁₂ and R₁₃ are each independently H or substituted or unsubstituted C₁-C₅ alkyl (e.g., ethyl, trifluoroethyl); or
R₁₂ and C3 are joined to form ring A and R₁₃ is R₃₀; or
R₁₂ and R₁₃ are joined to form ring B; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
   Ring A, C and E are each independently a substituted or unsubstituted single spiro or fused 3-8 membered heterocyclic rings;
   Ring B is a substituted or unsubstituted single, spiro or fused 3-8 membered heterocyclic ring; and
   Ring D is a substituted or unsubstituted C₃-C₈ cycloalkyl;

In some embodiments, R₁₂ of formula B and/or Bi is H. In some embodiments, R₁₂ is R₂₀. In other embodiments, R₁₂ is R₃₀. In some embodiments, R₁₂ is C₁-C₅ C(O)-alkyl. In some embodiments, R₁₂ is substituted or unsubstituted C₁-C₅ alkyl. In some embodiments, R₁₂ is unsubstituted C₁-C₅ alkyl. In some embodiments, the alkyl is ethyl. In some embodiments, R₁₂ is substituted C₁-C₅ alkyl. In some embodiments, the alkyl is trifluoroethyl.

In some embodiments, R₁₃ of formula B and/or Bi is H. In other embodiments, R₁₃ is R₃₀. In some embodiments, R₁₃ is substituted or unsubstituted C₁-C₅ alkyl. In some embodiments, R₁₃ is unsubstituted C₁-C₅ alkyl. In some embodiments, the alkyl is ethyl. In some embodiments, R₁₃ is substituted C₁-C₅ alkyl. In some embodiments, the alkyl is trifluoroethyl.

In some embodiments, R₆ of formula I, II and/or I(a)-I(i) is represented by formula B. In some embodiments, R₁₂ of formula B is R₂₀ or C₁-C₅ C(O)-alkyl, and R₁₃ is R₃₀. In some embodiments, R₁₂ and R₁₃ of formula B are both H. In some embodiments, R₁₂ and R₁₃ of formula B are each independently H or substituted or unsubstituted C₁-C₅ alkyl (e.g., ethyl, trifluoroethyl). In some embodiments, R₁₂ and R₁₃ of formula B are each independently H or trifluoroethyl. In some embodiments, R₁₂ and C3 of formula B are joined to form ring A and R₁₃ is R₃₀. In some embodiments, R₁₂ and R₁₃ of formula B are joined to form ring B. In some embodiments, R₁₂ and C1 of formula B are joined to form ring C and R₁₃ is R₃₀. In some embodiments, C1 and C3 of formula B are joined to form ring D and R₁₂ and R₁₃ of formula B are each independently R₃₀. In some embodiments, R₁₃ and C2 of formula B are joined to form ring E, m is 1, and R₁₂ of formula B is R₃₀. In some embodiments, R₁₂ and R₁₃ of formula B are joined to form ring B and C1 and C3 of formula B are joined to form ring D.

In some embodiments, R₆ of formula I, II and/or I(a)-I(h) is represented by formula Bi. In some embodiments, R₁₂ of formula Bi is R₂₀ or C₁-C₅ C(O)-alkyl, and R₁₃ is R₃₀. In some embodiments, R₁₂ and R₁₃ of formula Bi are both H. In some embodiments, R₁₂ and R₁₃ of formula Bi are each independently H or substituted or unsubstituted C₁-C₅ alkyl (e.g., ethyl, trifluoroethyl). In some embodiments, R₁₂ and R₁₃ of formula Bi are each independently H or trifluoroethyl. In some embodiments, R₁₂ and C3 of formula Bi are joined to form ring A and R₁₃ is R₃₀. In some embodiments, R₁₂ and R₁₃ of formula Bi are joined to form ring B. In some embodiments, R₁₂ and C1 of formula Bi are joined to form ring C and R₁₃ is R₃₀. In some embodiments, C1 and C3 of formula Bi are joined to form ring D and R₁₂ and R₁₃ of formula Bi are each independently R₃₀. In some embodiments, R₁₃ and C2 of formula Bi are joined to form ring E, m is 1, and R₁₂ of formula Bi is R₃₀. In some embodiments, R₁₂ and R₁₃ of formula Bi are joined to form ring B and C1 and C3 of formula Bi are joined to form ring D.

In some embodiments, R₆ of formula I(g) is represented by the structure of formula C:

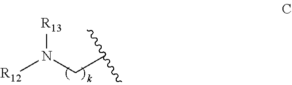

wherein

k is an integer number between 1 and 4;

R₁₂ and R₁₃ are each independently H, C₁-C₅ linear or branched, substituted or unsubstituted alkyl (e.g., ethyl, isopropyl), R₂₀, or

R₁₂ and R₁₃ are joined to form a substituted or unsubstituted 4-7 membered heterocyclic ring (e.g., piperidine, piperazine, pyrrolidine, oxa-6-azaspiro[3.3]heptane).

In some embodiments, k of formula C is 1. In some embodiments, k is 2. In some embodiments, k is 3. In some embodiments, k is 4.

In some embodiments, R₁₂ and R₁₃ of formula C are each independently H, C₁-C₅ linear or branched, substituted or unsubstituted alkyl (e.g., ethyl, isopropyl) or R₂₀; each represents a separate embodiment according to this invention. In some embodiments, R₁₂ and R₁₃ of formula C are both ethyls. In some embodiments, R₁₂ and R₁₃ of formula C are both isopropyls. In some embodiments, R₁₂ and R₁₃ of formula C are both alkyls.

In some embodiments, R₁₂ and R₁₃ of formula C are joined to form a substituted or unsubstituted 4-7 membered heterocyclic ring. In some embodiments, R₁₂ and R₁₃ of formula C are joined to form a piperidine, piperazine, pyrrolidine, oxa-6-azaspiro[3.3]heptane; each represents a separate embodiment according to this invention. In some embodiments the heterocyclic ring maybe further substituted with at least one substitution as defined herein for heterocyclic rings.

In some embodiments, R₆ of formula I(b) is represented by formula Bi and/or B and

R₁₂ of formula Bi and/or B is R₂₀ or C₁-C₅ C(O)-alkyl, and R₁₃ of formula Bi and/or B is R₃₀; or

R₁₂ and R₁₃ are both H, or

R₁₂ and R₁₃ are each independently H or trifluoroethyl; or

R₁₂ and C3 are joined to form ring A and R₁₃ is R₃₀; or

R₁₂ and R₁₃ are joined to form a substituted or unsubstituted pyrrolidine ring, piperazine, thiomorpholine 1,1-dioxide 2-oxa-6-azaspiro[3.3]heptane, pyrazole, imidazole, 2,5-diazabicyclo[2.2.1]heptane or a diazabicyclo[2.2.1]heptane; or

R₁₂ and C1 are joined to form ring C and R₁₃ is R₃₀; or

C3 are joined to form ring D and R₁₂ and R₁₃ are each independently R₃₀; or

R₁₃ and C2 are joined to form ring E, m is 1, and R₁₂ is R₃₀; or

R₁₂ and R₁₃ are joined to form ring B and C1 and C3 are joined to form ring D.

In some embodiments, R₆ of formula I(b) is represented by formula Bi and/or B and

R₁₂ of formula Bi and/or B is R₂₀ or C₁-C₅ C(O)-alkyl, and R₁₃ of formula Bi and/or B is R₃₀; or

R₁₂ and C3 are joined to form ring A and R₁₃ is R₃₀; or

R₁₂ and R₁₃ are joined to form a substituted or unsubstituted pyrrolidine ring, piperazine, thiomorpholine 1,1-dioxide 2-oxa-6-azaspiro[3.3]heptane, pyrazole, imidazole, 2,5-diazabicyclo[2.2.1]heptane or a diazabicyclo[2.2.1]heptane; or

R₁₂ and C1 are joined to form ring C and R₁₃ is R₃₀; or

C3 are joined to form ring D and R₁₂ and R₁₃ are each independently R₃₀; or

R₁₃ and C2 are joined to form ring E, m is 1, and R₁₂ is R₃₀; or

R₁₂ and R₁₃ are joined to form ring B and C1 and C3 are joined to form ring D.

In some embodiments, ring A of formula Bi, is a substituted or unsubstituted single spiro or fused 3-8 membered heterocyclic ring. In some embodiments, ring A, is an unsubstituted single 3-8 membered heterocyclic ring. In some embodiments, ring A, is an unsubstituted spiro 3-8 membered heterocyclic ring. In some embodiments, ring A, is an unsubstituted fused 3-8 membered heterocyclic ring. In some embodiments, ring A, is a substituted single 3-8 membered heterocyclic ring. In some embodiments, ring A, is a substituted spiro 3-8 membered heterocyclic ring. In some embodiments, ring A, is a substituted fused 3-8 membered heterocyclic ring. In some embodiments, ring A is: pyrrolidine, methylpyrrolidine, ethylpyrrolidine, 2-oxopyrrolidine, piperidine, methylpiperidine, methyl-2-oxopyrrolidine, pyran-azetidine, methyl-azetidine, azabicyclooctane, 2-azabicyclo[2.1.1]hexane, or 2-azaspiro[3.3]heptane; each represents a separate embodiment according to this invention. In some embodiments, ring A is: pyrrolidine, methylpyrrolidine, or ethylpyrrolidine; each represents a separate embodiment according to this invention.

In some embodiments, ring B of formula Bi, is a substituted or unsubstituted single spiro or fused 3-8 membered heterocyclic ring. In some embodiments, ring B, is an unsubstituted single 3-8 membered heterocyclic ring. In some embodiments, ring B, is an unsubstituted spiro 3-8 membered heterocyclic ring. In some embodiments, ring B, is an unsubstituted fused 3-8 membered heterocyclic ring. In some embodiments, ring B, is a substituted single 3-8 membered heterocyclic ring. In some embodiments, ring B, is a substituted spiro 3-8 membered heterocyclic ring. In some embodiments, ring B, is a substituted fused 3-8 membered heterocyclic ring. In some embodiments, ring B is: pyrrolidine, methylpyrrolidine, ethylpyrrolidine, 2-oxopyrrolidine, hydroxymethyl-pyrrolidine, piperidine, piperidin-2-one, 4-fluoropiperidin-2-one, piperidine-4-carbonitrile, methylpiperidine, fluoropiperidine, 4-fluoropiperidine, 4-fluoro-2-methylpiperidine, difluoropiperidine, piperazine, methyl-piperazine, dimethyl-pyrazole, methyl-2-oxopyrrolidine, pyran-, azetidine, methyl-azetidine, imidazole, azabicyclooctane, 2-azabicyclo[2.1.1]hexane, or 2-azaspiro[3.3]heptane, diazabicyclo[2.2.1]heptane, 2-methyl-2,5-diazabicyclo[2.2.1]heptane, thiomorpholine, or 1,1-dioxide-2-oxa-6-azaspiro[3.3]heptane; each represents a separate embodiment according to this invention. In some embodiments, ring B is: piperidine, methyl-piperidin, fluoropiperidine, difluoropiperidine, pyrrolidine, piperazine, methylpyrrolidine, thiomorpholine, methyl-piperazine, dimethyl-pyrazole, imidazole, 2-methyl-2,5-diazabicyclo[2.2.1]heptane, 1,1-dioxide-2-oxa-6-azaspiro[3.3]heptane, hydroxymethyl-pyrrolidine or diazabicyclo[2.2.1]heptane, 6-fluoro-3-azabicyclo[3.1.1]heptane; each represents a separate embodiment according to this invention.

In some embodiments, ring C of formula Bi, is a substituted or unsubstituted single spiro or fused 3-8 membered heterocyclic ring. In some embodiments, ring C, is an unsubstituted single 3-8 membered heterocyclic ring. In some embodiments, ring C, is an unsubstituted spiro 3-8 membered heterocyclic ring. In some embodiments, ring C, is an unsubstituted fused 3-8 membered heterocyclic ring. In some embodiments, ring C, is a substituted single 3-8 membered heterocyclic ring. In some embodiments, ring C, is a substituted spiro 3-8 membered heterocyclic ring. In some embodiments, ring C, is a substituted fused 3-8 membered heterocyclic ring. In some embodiments, ring C is: pyrrolidine, methylpyrrolidine, ethylpyrrolidine, 2-oxopyrrolidine, piperidine, methylpiperidine, methyl-2-oxopyrrolidine, pyran-azetidine, methyl-azetidine, azabicyclooctane, 2-azabicyclo[2.1.1]hexane, or 2-azaspiro[3.3]heptane; each represents a separate embodiment according to this invention. In some embodiments, ring C is: piperidine, pyrrolidine, methyl-2-oxopyrrolidine, pyran-pyrrolidine, methyl-azetidine, azabicyclooctane, 2-azabicyclo[2.1.1]hexane, or 2-azaspiro[3.3]heptane; each represents a separate embodiment according to this invention.

In some embodiments, ring D of formula Bi, is a substituted or unsubstituted C₃-C₈ cycloalkyl. In some embodiments, ring D, is a substituted C₃-C₈ cycloalkyl. In some embodiments, ring D, is an unsubstituted C₃-C₈ cycloalkyl. In some embodiments, ring D is cyclopropane, cyclobutene, cyclopentane, cyclohexane or cycloheptane; each represents a separate embodiment according to this invention.

In some embodiments, ring E of formula Bi, is a substituted or unsubstituted single spiro or fused 3-8 membered heterocyclic ring. In some embodiments, ring E, is an unsubstituted single 3-8 membered heterocyclic ring. In some embodiments, ring E, is an unsubstituted spiro 3-8 membered heterocyclic ring. In some embodiments, ring E, is an unsubstituted fused 3-8 membered heterocyclic ring. In some embodiments, ring E, is a substituted single 3-8 membered heterocyclic ring. In some embodiments, ring E, is a substituted spiro 3-8 membered heterocyclic ring. In some embodiments, ring E, is a substituted fused 3-8 membered heterocyclic ring. In some embodiments, ring E is: pyrrolidine, methylpyrrolidine, ethylpyrrolidine, 2-oxopyrrolidine, piperidine, methylpiperidine, methyl-2-oxopyrrolidine, pyran-azetidine, methyl-azetidine, azabicyclooctane, 2-azabicyclo[2.1.1]hexane, or 2-azaspiro[3.3]heptane; each represents a separate embodiment according to this invention. In some embodiments, ring E is: pyrrolidine, azetidine, ethylpyrrolidine, oxopyrrolidine, or methylpiperidine; each represents a separate embodiment according to this invention.

In some embodiments, R₆ of formula I(b) is F, Cl, Br, I, OH, SH, R₈—OH, R₈—SH, —R₈—O—R₁₀ (e.g., CH₂—O—CH₃), R₈—S—R₁₀ (e.g., (CH₂)₃—S—(CH₂)₂CH₃), R₈—NHC(O)—R₁₀, —O—R₈—R₁₀, R₈-(substituted or unsubstituted C₃-C₈ cycloalkyl) (e.g., CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), (CH₂)₃-pyran, CH₂-tetrahydrofurane, CH₂-dioxane, CH₂-methyl-THF, CH₂-tetrahydrofurane, CH₂-oxa-azaspirodecane, CH₂-azaspiroheptane, (CH₂)₃-dimethylpyrazole, CH₂-methyl-azetidine, CH₂-azaspiroheptane, CF₃, CD₃, OCD₃, CN, NO₂, —CH₂CN, —R₈CN, NH₂, NHR, N(R)₂, NH(R₁₀), N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), benzyl, methyl, ethyl, CH₂—OCH₂—CH₂—O—CH₃), C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy (e.g. methoxy, O—(CH₂)₂O—CH₃), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., methoxycyclopropyl, methylcyclobutyl, cyclopropyl, aminomethyl-cyclobutyl, methoxycyclobutyl, 2,3-dihydro-1H-indenol), substituted or unsubstituted 3-8 membered heterocyclic ring (e.g., trifluoromethyl-oxetane, hydroxy-tetrahydrofurane, 1-methylazepan-2-one, 3-azabicyclo[3.1.0]hexane), substituted or unsubstituted aryl, or substituted or unsubstituted benzyl; each represents a separate embodiment according to this invention. In some embodiments, R₆ may be further substituted with at least one substitution selected from: F, Cl, Br, I, C₁-C₅ linear or branched alkyl, OH, alkoxy (e.g., OMe), amide (e.g., C(O)N(R)₂), C(O)-alkyl, C(O)-pyrrolidine, C(O)-piperidine, N(R)₂ (e.g., N(CH₃)₂, NH₂), NH(R₁₀), N(R₁₀)(R₁₁), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN, and NO₂; each represents a separate embodiment according to this invention.

In some embodiments, R₆ of formula I(b) is —R₈—O—R₁₀. In some embodiments, —R₈—O—R₁₀ is CH₂—O—CH₃. In some embodiments, R₆ is R₈—S—R₁₀. In some embodiments, R₈—S—R₁₀ is (CH₂)₃—S—(CH₂)₂CH₃. In some embodiments, R₆ is R₈—NHC(O)—R₁₀. In some embodiments, R₆ is R₈-(substituted or unsubstituted C₃-C₈ cycloalkyl). In some embodiments, the R₈-(substituted or unsubstituted C₃-C₈ cycloalkyl) is CH₂-cyclobutanol, CH₂-difluorocyclopropyl, CH₂-methylcyclopropyl, CH₂-dimethylamino-cyclohexyl, (CH₂)₂-cyclopentanole, CH₂-cyclohexanol), (CH₂)₃-pyran, CH₂-tetrahydrofurane, CH₂-dioxane, CH₂-methyl-THF, CH₂-tetrahydrofurane, CH₂-oxa-azaspirodecane, CH₂-azaspiroheptane, (CH₂)₃-dimethylpyrazole, CH₂-methyl-azetidine, or CH₂-azaspiroheptane; each represents a separate embodiment according to this invention. In some embodiments, R₆ is C₁-C₅ linear or branched, substituted or unsubstituted alkyl. In some embodiments, R₆ is C₁-C₅ linear or branched, substituted alkyl. In some embodiments, the substituted alkyl is CH(CH₃)CH₂OCH₃, CH(CH₃)CH₂NH₂, CH(CH₃)C(O)N(CH₃)₂, CH₂—CH(OH)Ph, (CH₂)₃N(H)CH₂CH₃, CH(CH₃)(CH₂)₂OH, CH(CH₂OH)(CH₂CH₃), (CH₂)₃—OCH₃, (CH₂)₂—OCH₃, (CH₂)₂—OCH(CH₃)₂, CH(CH₂OH)(CH₂CH(CH₃)₂), CH₂CH(CH₃)(OCH₃), CH₂CH(N(CH₃)₂)(CH₂CH₃), CH₂—OCH₂—CH₂—O—CH₃ or benzyl; each represents a separate embodiment according to this invention. In some embodiments, R₆ is C₁-C₅ linear or branched, unsubstituted alkyl. In some embodiments, the unsubstituted alkyl is methyl, ethyl, propyl, isopropyl, butyl, isobutyl, tert-butyl, pentyl, or neopentyl; each represents a separate embodiment according to this invention. In some embodiments, R₆ is substituted or unsubstituted C₃-C₈ cycloalkyl. In some embodiments, R₆ is substituted C₃-C₈ cycloalkyl. In some embodiments, the substituted cycloalkyl is methoxycyclopropyl, methylcyclobutyl, aminomethyl-cyclobutyl, or methoxycyclobutyl, 2,3-dihydro-1H-indenol; each represents a separate embodiment according to this invention. In some embodiments, R₆ is unsubstituted C₃-C₈ cycloalkyl. In some embodiments, the unsubstituted cycloalkyl is cyclopropyl, cyclobutyl, cyclopentyl, cyclohexyl, cycloheptyl or cyclooctyl; each represents a separate embodiment according to this invention. In some embodiments, R₆ is substituted or unsubstituted 3-8 membered heterocyclic ring. In some embodiments, the substituted heterocyclic ring trifluoromethyl-oxetane, hydroxy-tetrahydrofurane, 1-methylazepan-2-one, or 3-azabicyclo[3.1.0]hexane; each represents a separate embodiment according to this invention.

In some embodiments, R₇ of formula I, II, I(a)-I(f) and/or I(i) is H, F, Cl, Br, I, OH, O—R₂₀, SH, R₈—OH, R₈—SH, SR₁₀, —R₈—O—R₁₀, —R₈—S—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted −47 membered heterocyclic ring, substituted or unsubstituted aryl, or substituted or unsubstituted benzyl; each represents a separate embodiment according to this invention. In some embodiments, R₇ is further substituted with at least one substitution selected from: F, Cl, Br, I, C₁-C₅ linear or branched alkyl, OH, alkoxy (e.g., OMe), amide (e.g., C(O)N(R)₂), C(O)-alkyl, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₇ of formula I, II, I(b), I(d)-I(f) and/or I(i) is H. In some embodiments, R₇ is F. In some embodiments, R₁₇ is Cl. In some embodiments, R₇ is Br. In some embodiments, R₇ is I. In some embodiments, R₇ is OH. In some embodiments, R₇ is O—R₂₀. In some embodiments, R₇ is CF₃. In some embodiments, R₁₇ is CN. In some embodiments, R₇ is NH₂. In some embodiments, R₇ is NHR. In some embodiments, R₇ is N(R)₂. In some embodiments, R₇ is NH(R₁₀). In some embodiments, R₇ is N(R₁₀)(R₁₁). In some embodiments, R₇ is NHC(O)—R₁₀. In some embodiments, R₇ is COOH. In some embodiments, R₇ is —C(O)Ph. In some embodiments, R₇ is C(O)O—R₁₀. In some embodiments, R₇ is C(O)H. In some embodiments, R₇ is C(O)—R₁₀. In some embodiments, R₇ is C₁-C₅ linear or branched C(O)-haloalkyl. In some embodiments, R₇ is —C(O)NH₂. In some embodiments, R₇ is C(O)NHR. In some embodiments, C(O)NHR is C(O)NH(CH₃). In some embodiments, R₇ is C(O)N(R₁₀)(R₁₁). In some embodiments, C(O)N(R₁₀)(R₁₁) is C(O)NH(CH₃), C(O)NH(CH₂CH₂OCH₃), or C(O)NH(CH₂CH₂OH); each represents a separate embodiment according to this invention. In some embodiments, R₇ is SO₂R. In some embodiments, R₇ is C₁-C₅ linear or branched, substituted or unsubstituted alkyl. In some embodiments, the alkyl is methylimidazole, methyl, ethyl, propyl, isopropyl, butyl, isobutyl, tert-butyl, pentyl, neopentyl or hexyl; each represents a separate embodiment according to this invention. In some embodiments, R₇ is C₁-C₅ linear or branched, or C₃-C₈ cyclic haloalkyl. In some embodiments, R₇ is C₁-C₅ linear haloalkyl. In some embodiments, the haloalkyl is CHF₂. In some embodiments, R₇ is C₁-C₅ branched haloalkyl. In some embodiments, R₁₇ is C₃-C₈ cyclic haloalkyl. In some embodiments, R₁₇ is C₁-C₅ linear or branched, or C₃-C₈ cyclic alkoxy optionally wherein at least one methylene group (CH₂) in the alkoxy is replaced with an oxygen atom. In some embodiments, R₇ is C₁-C₅ linear alkoxy. In some embodiments, the alkoxy is methoxy. In some embodiments, the alkoxy is ethoxy. In some embodiments, R₇ is C₁-C₅ branched alkoxy. In some embodiments, R₇ is C₃-C₈ cyclic alkoxy. In some embodiments, R₇ is C₁-C₅ linear or branched thioalkyl. In some embodiments, R₇ is C₁-C₅ linear or branched haloalkoxy. In some embodiments, R₇ is C₁-C₅ linear haloalkoxy. In some embodiments, R₇ is C₁-C₅ branched haloalkoxy. In some embodiments, R₇ is C₁-C₅ linear or branched alkoxyalkyl. In some embodiments, R₇ is substituted or unsubstituted C₃-C₈ cycloalkyl. In some embodiments, the cycloalkyl is cyclopropyl, cyclopropanol, cyclobutyl, cyclopentyl, cyclohexyl, cycloheptyl or cyclooctyl; each represents a separate embodiment according to this invention. In some embodiments, R₇ is substituted or unsubstituted 4-7 membered heterocyclic ring. In some embodiments, R₇ is unsubstituted 4-7 membered heterocyclic ring. In some embodiments, R₇ is substituted 4-7 membered heterocyclic ring. In some embodiments, the heterocyclic ring is morpholine, tetrahydrofuran, tetrahydropyran, oxetane, oxetan-3-ol, pyrrolidine, 1-methylpyrrolidine, pyrrolidin-2-one, pyrrolidinone, imidazole, pyrazole, piperazine, piperidine, piperidine-4-carbonitrile, 4-fluoropiperidine, oxadiazole, triazole, or 2-oxopyrrolidine; each represents a separate embodiment according to this invention. In some embodiments, R₇ is R₈-(substituted or unsubstituted single, fused or spiro 3-8 membered heterocyclic ring). In some embodiments, R₇ is R₈-(unsubstituted single 3-8 membered heterocyclic ring). In some embodiments, R₇ is R₈-(unsubstituted fused 3-8 membered heterocyclic ring). In some embodiments, R₇ is R₈-(unsubstituted spiro 3-8 membered heterocyclic ring). In some embodiments, R₁₇ is R₈-(substituted single 3-8 membered heterocyclic ring). In some embodiments, R₇ is R₈-(substituted fused 3-8 membered heterocyclic ring). In some embodiments, R₇ is R₈-(substituted spiro 3-8 membered heterocyclic ring). In some embodiments, the heterocyclic ring may be saturated. In some embodiments, the heterocyclic ring may be unsaturated. In some embodiments, the hetrocyclic ring may be aromatic. In some embodiments, R₇ is substituted or unsubstituted aryl. In some embodiments, R₇ is phenyl. In some embodiments, R₇ may be further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₇ of formula I(a) is O—R₂₀. In some embodiments, R₇ is substituted or unsubstituted 4-7 membered heterocyclic ring. In some embodiments, R₇ is unsubstituted 4-7 membered heterocyclic ring. In some embodiments, R₇ is substituted 4-7 membered heterocyclic ring. In some embodiments, the heterocyclic ring is morpholine, pyran, oxetane, pyrrolidine, imidazole, piperazine, piperidine, diaoxazole, triazole, or 2-oxopyrrolidine; each represents a separate embodiment according to this invention. In some embodiments, R₇ is substituted or unsubstituted aryl. In some embodiments, R₁₇ is phenyl. In some embodiments, R₁₇ may be further substituted with at least one substitution selected from F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₇ of formula I(c) is not H, F, Cl, C₁-C₅ linear or branched, or C₃-C₈ cyclic alkoxy, C₁-C₅ linear or branched haloalkoxy or C₁-C₅ linear or branched, substituted or unsubstituted alkyl.

In some embodiments, R₇ of formula I, II, I(a)-I(f) and/or I(i) is represented by the structure of formula A:

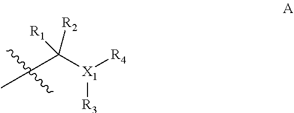

wherein
X₁ is N or O;
R₁ and R₂ are each independently H, F, or CF₃; or R₁ and R₂ are joined to form ═O or a C₃-C₈ carbocyclic or heterocyclic ring (e.g., cyclopropyl);
R₃ and R₄ are each independently H, Me, substituted or unsubstituted C₁-C₅ alkyl (e.g., methoxyethyl, methylaminoethyl, aminoethyl), substituted or unsubstituted C₃-C₈ cycloalkyl (e.g., cyclopropyl), substituted or unsubstituted 5-6 membered heterocyclic ring (e.g., pyrrolidine, methylpyrrolidine, piperidine), or R₂₀; or R₃ and R₄ are joined to form a 3-8 membered heterocyclic ring (e.g., pyrrolidine, 2-oxopyrrolidine, piperidine, morpholine, piperazine);
wherein if X₁ is O then R₄ is absent;

In some embodiments, X₁ of formula A is N. In other embodiments X₁ is O.

In some embodiments, R₁ of formula A is H. In other embodiments R₁ is F. In other embodiments R₁ is CF₃.

In some embodiments, R₂ of formula A is H. In other embodiments R₂ is F. In other embodiments R₂ is CF₃.

In some embodiments, R₁ and R₂ of formula A are joined to form ═O. In other embodiments, R₁ and R₂ are joined to form a C₃-C₈ carbocyclic or heterocyclic ring. In other embodiments, R₁ and R₂ are joined to form a C₃-C₈ carbocyclic ring. In some embodiments, the carbocyclic ring is cyclopropyl. In other embodiments, R₁ and R₂ are joined to form a 3-8 membered heterocyclic ring.

In some embodiments, R₁ and R₂ of formula A of formula I(a), are not joined to form ═O.

In some embodiments, R₃ of formula A is H. In some embodiments, R₃ is methyl. In some embodiments, R₃ is substituted or unsubstituted C₁-C₅ alkyl. In some embodiments, the alkyl is methoxyethylene, methylaminoethylene, aminoethylene; each represents a separate embodiment according to this invention. In some embodiments, R₃ is substituted or unsubstituted C₃-C₈ cycloalkyl. In some embodiments, the cycloalkyl is cyclopropyl. In some embodiments, R₃ is substituted or unsubstituted 5-7 membered heterocyclic ring. In some embodiments, the heterocyclic ring is pyrrolidine, methylpyrrolidine, or piperidine; each represents a separate embodiment according to this invention. In some embodiments, R₃ is R₂₀ as defined hereinbelow.

In some embodiments, R₄ of formula A is H. In some embodiments, R₄ is methyl. In some embodiments, R₄ is substituted or unsubstituted C₁-C₅ alkyl. In some embodiments, the alkyl is methoxyethylene, methylaminoethylene, aminoethylene; each represents a separate embodiment according to this invention. In some embodiments, R₄ is substituted or unsubstituted C₃-C₈ cycloalkyl. In some embodiments, the cycloalkyl is cyclopropyl. In some embodiments, R₄ is substituted or unsubstituted 5-7 membered heterocyclic ring. In some embodiments, the heterocyclic ring is pyrrolidine, methylpyrrolidine, or piperidine; each represents a separate embodiment according to this invention. In some embodiments, R₄ is R₂₀ as defined hereinbelow.

In some embodiments, R₃ and R₄ of formula A are joined to form a 3-8 membered heterocyclic ring. In some embodiments, the heterocyclic ring is imidazole, pyrrolidine, 2-oxopyrrolidine, piperidine, morpholine, or piperazine; each represents a separate embodiment according to this invention.

In some embodiments, if X₁ of formula A is O then R₄ is absent.

In some embodiments, R₇ of formula I(a) is O—R₂₀, substituted or unsubstituted 4-7 membered heterocyclic ring (e.g., morpholine, pyran, oxetane, pyrrolidine, imidazole, piperazine, piperidine, diaoxazole, triazole, 2-oxopyrrolidine), or substituted or unsubstituted aryl. In some embodiments, R₇ of formula I(a) is represented by formula A, wherein X₁, R₁, R₂, R₃ and R₄ are as defined above except that R₁ and R₂ cannot be joined to form ═O.

In some embodiments, R₇′ of formula I(c) is not H.

In some embodiments, R₇′ of formula I, II, I(a)-I(b) and/or I(d)-I(i) is H. In some embodiments, R₇′ of formula I, II and/or I(a)-I(i) is F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(Rn), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted 3-8 membered heterocyclic ring, substituted or unsubstituted aryl, or substituted or unsubstituted benzyl; each represents a separate embodiment according to this invention. In some embodiments, R₇′ is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₇′ of formula I, II and/or I(a)-I(i) is H. In some embodiments, R₇′ is F. In some embodiments, R₇′ is Cl. In some embodiments, R₇′ is Br. In some embodiments, R₇′ is I. In some embodiments, R₇′ is CF₃. In some embodiments, R₇′ is C₁-C₅ linear or branched, substituted or unsubstituted alkyl. In some embodiments, R₇′ is C₁-C₅ linear or branched unsubstituted alkyl. In some embodiments, the alkyl is isopropyl, methyl, ethyl; each represents a separate embodiment according to this invention. In some embodiments, R₇′ is C₁-C₅ linear or branched substituted alkyl. In some embodiments, R₇′ is isopropyl. In some embodiments, R₇′ is methyl. In some embodiments, R₇′ is ethyl. In some embodiments, R₇′ is C₁-C₅ linear or branched, or C₃-C₈ cyclic haloalkyl. In some embodiments, R₇′ is C₁-C₅ linear or branched haloalkyl. In some embodiments, the haloalkyl is CHF₂. In some embodiments, R₇′ is C₃-C₈ cyclic haloalkyl. In some embodiments, R₇′ is substituted or unsubstituted C₃-C₈ cycloalkyl. In some embodiments, the cycloalkyl is cyclopropyl.

In some embodiments, R₇ and R₇′ of formula I, II and/or I(a)-I(f) are joined to form a 5 or 6 membered substituted or unsubstituted, saturated, unsaturated or aromatic, carbocyclic or heterocyclic ring. In some embodiments, R₇ and R₇′ are joined to form a 5 membered unsubstituted saturated or unsaturated carbocyclic ring. In some embodiments, R₇ and R₇′ are joined to form 6 membered unsubstituted saturated or unsaturated carbocyclic ring. In some embodiments, R₇ and R₇′ are joined to form a 5 membered substituted saturated or unsaturated carbocyclic ring. In some embodiments, R₇ and R₇′ are joined to form 6 membered substituted saturated or unsaturated carbocyclic ring. In some embodiments, R₇ and R₇′ are joined to form a 6 membered substituted or unsubstituted, aromatic, carbocyclic ring. In some embodiments, R₇ and R₇′ are joined to form a 5 or 6 membered substituted or unsubstituted, aromatic, heterocyclic ring. In some embodiments, R₇ and R₇′ are joined to form a 5 or 6 membered substituted or unsubstituted, heterocyclic ring. In some embodiments, R₇ and R₇′ are joined to form a 6 membered substituted or unsubstituted, heterocyclic ring. In some embodiments, R₇ and R₇′ are joined to form a piperidine. In some embodiments, R₇ and R₇′ are joined to form a tetrahydropyran.

In some embodiments, R₇ and R₇′ are joined to form a 5 membered substituted or unsubstituted, heterocyclic ring. In some embodiments, R₇ and R₇′ are joined to form a pyrrolidine. In some embodiments, R₇ and R₇′ are joined to form a tetrahydrofuran.

In some embodiments, R₇ and R₇′ of formula I(c) are different. In some embodiments, R₇ and R₇′ of formula I(c) are not H, F, Cl, C₁-C₅ linear or branched, or C₃-C₈ cyclic alkoxy, C₁-C₅ linear or branched haloalkoxy or C₁-C₅ linear or branched, substituted or unsubstituted alkyl; each represents a separate embodiment according to this invention.

In some embodiments, R₇″ of formula I(i) is H. In some embodiments, R₇″ of formula I(i) is F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted 3-8 membered heterocyclic ring, substituted or unsubstituted aryl, or substituted or unsubstituted benzyl; each represents a separate embodiment according to this invention. In some embodiments, R₇″ is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₇″ of formula I(i) is H. In some embodiments, R₇″ is F. In some embodiments, R₇″ is Cl. In some embodiments, R₇″ is Br. In some embodiments, R₇″ is I. In some embodiments, R₇″ is CF₃. In some embodiments, R₇″ is C₁-C₅ linear or branched, substituted or unsubstituted alkyl. In some embodiments, R₇′″ is C₁-C₅ linear or branched unsubstituted alkyl. In some embodiments, the alkyl is isopropyl, methyl, ethyl; each represents a separate embodiment according to this invention. In some embodiments, R₇″ is C₁-C₅ linear or branched substituted alkyl. In some embodiments, R₇″ is isopropyl. In some embodiments, R₇″ is methyl. In some embodiments, R₇″ is ethyl. In some embodiments, R₇″ is C₁-C₅ linear or branched, or C₃-C₈ cyclic haloalkyl. In some embodiments, R₇″ is C₁-C₅ linear or branched haloalkyl. In some embodiments, the haloalkyl is CHF₂. In some embodiments, R₇″ is C₃-C₈ cyclic haloalkyl. In some embodiments, R₇″ is substituted or unsubstituted C₃-C₈ cycloalkyl. In some embodiments, the cycloalkyl is cyclopropyl.

In some embodiments, R₇′″ of formula I(i) is H. In some embodiments, R₇′″ of formula I(i) is F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted 3-8 membered heterocyclic ring, substituted or unsubstituted aryl, or substituted or unsubstituted benzyl; each represents a separate embodiment according to this invention. In some embodiments, R₇′″ is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₇′″ of formula I(i) is H. In some embodiments, R₇′″ is F. In some embodiments, R₇′″ is Cl. In some embodiments, R₇′″ is Br. In some embodiments, R₇′″ is I. In some embodiments, R₇′″ is CF₃. In some embodiments, R₇′″ is C₁-C₅ linear or branched, substituted or unsubstituted alkyl. In some embodiments, R₇′″ is C₁-C₅ linear or branched unsubstituted alkyl. In some embodiments, the alkyl is isopropyl, methyl, ethyl; each represents a separate embodiment according to this invention. In some embodiments, R₇′″ is C₁-C₅ linear or branched substituted alkyl. In some embodiments, R₇′″ is isopropyl. In some embodiments, R₇′″ is methyl. In some embodiments, R₇′″ is ethyl. In some embodiments, R₇′″ is C₁-C₅ linear or branched, or C₃-C₈ cyclic haloalkyl. In some embodiments, R₇′″ is C₁-C₅ linear or branched haloalkyl. In some embodiments, the haloalkyl is CHF₂. In some embodiments, R₇′″ is C₃-C₈ cyclic haloalkyl. In some embodiments, R₇′″ is substituted or unsubstituted C₃-C₈ cycloalkyl. In some embodiments, the cycloalkyl is cyclopropyl.

In some embodiments, R₇″″ of formula I(i) is H. In some embodiments, R₇″″ of formula I(i) is F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted 3-8 membered heterocyclic ring, substituted or unsubstituted aryl, or substituted or unsubstituted benzyl; each represents a separate embodiment according to this invention. In some embodiments, R₇″″ is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₇″″ of formula I(i) is H. In some embodiments, R₇″″ is F. In some embodiments, R₇″″ is Cl. In some embodiments, R₇″″ is Br. In some embodiments, R₇″″ is I. In some embodiments, R₇″″ is CF₃. In some embodiments, R₇″″ is C₁-C₅ linear or branched, substituted or unsubstituted alkyl. In some embodiments, R₇″″ is C₁-C₅ linear or branched unsubstituted alkyl. In some embodiments, the alkyl is isopropyl, methyl, ethyl; each represents a separate embodiment according to this invention. In some embodiments, R₇″″ is C₁-C₅ linear or branched substituted alkyl.

In some embodiments, R₇″″ is isopropyl. I'n some embodiments, R₇″″ is methyl. In some embodiments, R₇″″ is ethyl. In some embodiments, R₇″″ is C₁-C₅ linear or branched, or C₃-C₈ cyclic haloalkyl. In some embodiments, R₇″″ is C₁-C₅ linear or branched haloalkyl. In some embodiments, the haloalkyl is CHF₂. In some embodiments, R₇″″ is C₃-C₈ cyclic haloalkyl. In some embodiments, R₇″″ is substituted or unsubstituted C₃-C₈ cycloalkyl. In some embodiments, the cycloalkyl is cyclopropyl.

In some embodiments, R₇′ and R₇″ of formula I(i) are joined to form a 3-8 membered substituted or unsubstituted, saturated, unsaturated or aromatic, carbocyclic or heterocyclic ring. In some embodiments, R₇′ and R₇″ are joined to form a 5 membered unsubstituted saturated or unsaturated carbocyclic ring. In some embodiments, R₇′ and R₇″ are joined to form a cyclopentane. In some embodiments, R₇′ and R₇″ are joined to form 6 membered unsubstituted saturated or unsaturated carbocyclic ring. In some embodiments, R₇′ and R₇″ are joined to form a cyclohexane. In some embodiments, R₇′ and R₇″ are joined to form a 5 membered substituted saturated or unsaturated carbocyclic ring. In some embodiments, R₇′ and R₇″ are joined to form 6 membered substituted saturated or unsaturated carbocyclic ring. In some embodiments, R₇′ and R₇″ are joined to form a 6 membered substituted or unsubstituted, aromatic, carbocyclic ring. In some embodiments, R₇′ and R₇″ are joined to form a 5 or 6 membered substituted or unsubstituted, aromatic, heterocyclic ring. In some embodiments, R₇′ and R₇″ are joined to form a 5 or 6 membered substituted or unsubstituted, heterocyclic ring. In some embodiments, R₇′ and R₇″ are joined to form a 6 membered substituted or unsubstituted, heterocyclic ring. In some embodiments, R₇′ and R₇″ are joined to form a piperidine. In some embodiments, R₇′ and R₇″ are joined to form a tetrahydropyran. In some embodiments, R₇′ and R₇″ are joined to form a 5 membered substituted or unsubstituted, heterocyclic ring. In some embodiments, R₇′ and R₇″ are joined to form a tetrahydrofuran. In some embodiments, R₇′ and R₇″ are joined to form a pyrrolidine.

In some embodiments, R₇′ and R₇″ of formula I(i) are different. In some embodiments, R₇′ and R₇″ of formula I(i) are not H, F, Cl, C₁-C₅ linear or branched, or C₃-C₈ cyclic alkoxy, C₁-C₅ linear or branched haloalkoxy or C₁-C₅ linear or branched, substituted or unsubstituted alkyl; each represents a separate embodiment according to this invention.

In some embodiments, R₇″ and R₇ of formula I(i) are joined to form a 3-8 membered substituted or unsubstituted, saturated, unsaturated or aromatic, carbocyclic or heterocyclic ring. In some embodiments, R₇″ and R₇ are joined to form a 5 membered unsubstituted saturated or unsaturated carbocyclic ring. In some embodiments, R₇″ and R₇ are joined to form a cyclopentane. In some embodiments, R₇″ and R₇ are joined to form 6 membered unsubstituted saturated or unsaturated carbocyclic ring. In some embodiments, R₇″ and R₇ are joined to form a cyclohexane. In some embodiments, R₇″ and R₇ are joined to form a 5 membered substituted saturated or unsaturated carbocyclic ring. In some embodiments, R₇″ and R₇ are joined to form 6 membered substituted saturated or unsaturated carbocyclic ring. In some embodiments, R₇′ and R₇″ are joined to form a 6 membered substituted or unsubstituted, aromatic, carbocyclic ring. In some embodiments, R₇″ and R₇ are joined to form a 5 or 6 membered substituted or unsubstituted, aromatic, heterocyclic ring. In some embodiments, R₇″ and R₇ are joined to form a 5 or 6 membered substituted or unsubstituted, heterocyclic ring. In some embodiments, R₇″ and R₇ are joined to form a 6 membered substituted or unsubstituted, heterocyclic ring. In some embodiments, R₇″ and R₇ are joined to form a piperidine. In some embodiments, R₇″ and R₇ are joined to form a tetrahydropyran. In some embodiments, R₇″ and R₇ are joined to form a 5 membered substituted or unsubstituted, heterocyclic ring. In some embodiments, R₇″ and R₇ are joined to form a tetrahydrofuran. In some embodiments, R₇″ and R₇ are joined to form a pyrrolidine.

In some embodiments, R₇″ and R₇ of formula I(i) are different. In some embodiments, R₇″ and R₇ of formula I(i) are not H, F, Cl, C₁-C₅ linear or branched, or C₃-C₈ cyclic alkoxy, C₁-C₅ linear or branched haloalkoxy or C₁-C₅ linear or branched, substituted or unsubstituted alkyl; each represents a separate embodiment according to this invention.

In some embodiments, R₇ and R₇′″ of formula I(i) are joined to form a 3-8 membered substituted or unsubstituted, saturated, unsaturated or aromatic, carbocyclic or heterocyclic ring. In some embodiments, R₇ and R₇′″ are joined to form a 5 membered unsubstituted saturated or unsaturated carbocyclic ring. In some embodiments, R₇ and R₇′″ are joined to form 6 membered unsubstituted saturated or unsaturated carbocyclic ring. In some embodiments, R₇ and R₇′″ are joined to form a 5 membered substituted saturated or unsaturated carbocyclic ring. In some embodiments, R₇ and R₇′″ are joined to form 6 membered substituted saturated or unsaturated carbocyclic ring. In some embodiments, R₇ and R₇′″ are joined to form a 6 membered substituted or unsubstituted, aromatic, carbocyclic ring.

In some embodiments, R₇ and R₇′″ are joined to form a 5 or 6 membered substituted or unsubstituted, aromatic, heterocyclic ring. In some embodiments, R₇ and R₇′″ are joined to form a 5 or 6 membered substituted or unsubstituted, heterocyclic ring. In some embodiments, R₇ and R₇′″ are joined to form a 6 membered substituted or unsubstituted, heterocyclic ring. In some embodiments, R₇ and R₇′″ are joined to form a piperidine. In some embodiments, R₇ and R₇′″ are joined to form a tetrahydrofuran. In some embodiments, R₇ and R₇′″ are joined to form a tetrahydropyran. In some embodiments, R₇ and R₇′″ are joined to form a 5 membered substituted or unsubstituted, heterocyclic ring. In some embodiments, R₇ and R₇′″ are joined to form a pyrrolidine. In some embodiments, R₇ and R₇′″ are joined to form a cyclopentane. In some embodiments, R₇ and R₇′″ are joined to form a cyclohexane.

In some embodiments, R₇ and R₇′″ of formula I(i) are different. In some embodiments, R₇ and R₇′″ of formula I(i) are not H, F, Cl, C₁-C₅ linear or branched, or C₃-C₈ cyclic alkoxy, C₁-C₅ linear or branched haloalkoxy or C₁-C₅ linear or branched, substituted or unsubstituted alkyl; each represents a separate embodiment according to this invention.

In some embodiments, R₇′″ and R₇″″ of formula I(i) are joined to form a 3-8 membered substituted or unsubstituted, saturated, unsaturated or aromatic, carbocyclic or heterocyclic ring. In some embodiments, R₇′″ and R₇″″ are joined to form a 5 membered unsubstituted saturated or unsaturated carbocyclic ring. In some embodiments, R₇′″ and R₇″″ are joined to form 6 membered unsubstituted saturated or unsaturated carbocyclic ring. In some embodiments, R₇′″ and R₇″″ are joined to form a 5 membered substituted saturated or unsaturated carbocyclic ring. In some embodiments, R₇′″ and R₇″″ are joined to form 6 membered substituted saturated or unsaturated carbocyclic ring. In some embodiments, R₇′″ and R₇″″ are joined to form a 6 membered substituted or unsubstituted, aromatic, carbocyclic ring. In some embodiments, R₇′″ and R₇″″ are joined to form a 5 or 6 membered substituted or unsubstituted, aromatic, heterocyclic ring. In some embodiments, R₇′″ and R₇″″ are joined to form a 5 or 6 membered substituted or unsubstituted, heterocyclic ring. In some embodiments, R₇′″ and R₇″″ are joined to form a 6 membered substituted or unsubstituted, heterocyclic ring. In some embodiments, R₇′″ and R₇″″ are joined to form a piperidine. In some embodiments, R₇″ and R₇″″ are joined to form a tetrahydrofuran. In some embodiments, R₇′″ and R₇″″ are joined to form a tetrahydropyran. In some embodiments, R₇′″ and R₇″″ are joined to form a 5 membered substituted or unsubstituted, heterocyclic ring. In some embodiments, R₇′″ and R₇″″ are joined to form a pyrrolidine. In some embodiments, R₇′″ and R₇″″ are joined to form a cyclopentane. In some embodiments, R₇′″ and R₇″″ are joined to form a cyclohexane.

In some embodiments, R₇′″ and R₇″ of formula I(i) are different. In some embodiments, R₇′″ and R₇″″ of formula I(i) are not H, F, Cl, C₁-C₅ linear or branched, or C₃-C₈ cyclic alkoxy, C₁-C₅ linear or branched haloalkoxy or C₁-C₅ linear or branched, substituted or unsubstituted alkyl; each represents a separate embodiment according to this invention.

In some embodiments, at least two of R₇, R₇′, R₇″, R₇′″ and R₇″″ are not H.

In some embodiments, R₃₀ of formula I, II and/or I(a)-I(i) is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl, C₁-C₅ linear or branched alkoxy, C₁-C₅ linear or branched haloalkyl, R₈-aryl, —R₈—O—R₈—O—R₁₀, —R₈—O—R₁₀, —R₈-R₁₀, substituted or unsubstituted aryl, or substituted or unsubstituted heteroaryl; each represents a separate embodiment according to this invention. In some embodiments, R₃₀ is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention. In some embodiments, R₃₀ is H. In some embodiments, R₃₀ is R₂₀.

In some embodiments, R of formula I, II and/or I(a)-I(i) is H, F, Cl, Br, I, OH, SH, OH, alkoxy, NH(R₁₀), N(R₁₀)(R₁₁), CF₃, CN, NO₂, COOH, C₁-C₅ linear or branched, substituted or unsubstituted alkyl, C₁-C₅ linear or branched alkoxy, C₁-C₅ linear or branched haloalkyl, R₈-aryl, —R₈—O—R₈—O—R₁₀, —R₈—O—R₁₀, —R₈-R₁₀, substituted or unsubstituted aryl, or substituted or unsubstituted heteroaryl; each represents a separate embodiment according to this invention. In some embodiments, R is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention. In some embodiments, R is H. In some embodiments, R is NH(R₁₀). In some embodiments, R is NH—CH₂-cyclopropyl. In some embodiments, R is C₁-C₅ linear or branched, substituted or unsubstituted alkyl. In some embodiments, R is methyl. In some embodiments, R is ethyl. In some embodiments, R is propyl. In some embodiments, R is isopropyl. In some embodiments, R is butyl. In some embodiments, R is substituted alkyl. In some embodiments, R is CH₂—OH. In some embodiments, R is CH₂—CH₂—OH. In some embodiments, R is C₃-C₈ substituted or unsubstituted cycloalkyl. In some embodiments, R is cyclopropyl. In some embodiments, R is C₁-C₅ linear or branched alkoxy. In some embodiments, R is methoxy. In some embodiments, R is ethoxy. In some embodiments, R is propoxy. In some embodiments, R is isopropoxy. In some embodiments, R is COOH.

In various embodiments, each R₈ of compound of formula I, II and/or I(a)-I(i) is independently CH₂. In some embodiments, R₈ is CH₂CH₂. In some embodiments, R₈ is CH₂CH₂CH₂. In some embodiments, R₈ is CH₂CH₂CH₂CH₂.

In some embodiments, p of formula I, II and/or I(a)-I(i) is 1. In other embodiments, p is 2. In other embodiments, p is 3. In some embodiments, p is 4. In some embodiments, p is 5. In some embodiments, p is between 1 and 3. In some embodiments, p is between 1 and 5. In some embodiments, p is between 1 and 10.

In some embodiments, R₉ of formula I, II and/or I(a)-I(i) is C≡C. In some embodiments, R₉ is C≡C—C≡C. In some embodiments, R₉ is CH═CH. In some embodiments, R₉ is CH═CH—CH═CH.

In some embodiments, q of formula I, II and/or I(a)-I(i) is 2. In some embodiments, q is 4. In some embodiments, q is 6. In some embodiments, q is 8. In some embodiments, q is between 2 and 6.

In some embodiments, R₁₀ of formula I, II and/or I(a)-I(i) is H, C₁-C₅ substituted or unsubstituted linear or branched alkyl (e.g., methyl, ethyl, CH₂-cyclopropyl, CH₂—CH₂—O—CH₃), C₁-C₅ substituted or unsubstituted linear or branched haloalky, CH₂CF₃, C₁-C₅ linear or branched alkoxy (e.g., O—CH₃), R₂₀, C(O)R, or S(O)₂R; each represents a separate embodiment according to this invention. In some embodiments, R₁₀ is H. In some embodiments, R₁₀ is C₁-C₅ substituted or unsubstituted linear or branched alkyl. In some embodiments, R₁₀ is C₁-C₅ unsubstituted linear or branched alkyl. In other embodiments, R₁₀ is CH₃. In other embodiments, R₁₀ is CH₂CH₃. In other embodiments, R₁₀ is CH₂CH₂CH₃. In some embodiments, R₁₀ is isopropyl. In some embodiments, R₁₀ is butyl. In some embodiments, R₁₀ is isobutyl. In some embodiments, R₁₀ is t-butyl. In some embodiments, R₁₀ is pentyl. In some embodiments, R₁₀ is isopentyl. In some embodiments, R₁₀ is neopentyl. In some embodiments, R₁₀ is benzyl. In some embodiments, R₁₀ is C₁-C₅ substituted linear or branched alkyl. In other embodiments, R₁₀ is CH₂—CH₂—O—CH₃. In other embodiments, R₁₀ is CH₂CF₃. In other embodiments, R₁₀ is C₁-C₅ substituted or unsubstituted linear or branched haloalkyl. In other embodiments, R₁₀ is C₁-C₅ linear or branched alkoxy. In other embodiments, R₁₀ is O—CH₃. In other embodiments, R₁₀ is R₂₀. In other embodiments, R₁₀ is C(O)R. In other embodiments, R₁₀ is S(O)₂R. In some embodiments, R₁₀ is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₁₁ of formula I, II and/or I(a)-I(i) is H, C₁-C₅ substituted or unsubstituted linear or branched alkyl (e.g., methyl, ethyl, CH₂—CH₂—O—CH₃, CH₂CF₃, C₁-C₅ linear or branched alkoxy (e.g., O—CH₃), C(O)R, or S(O)₂R; each represents a separate embodiment according to this invention. In some embodiments, R₁₁ is H. In some embodiments, R₁₁ is C₁-C₅ substituted or unsubstituted linear or branched alkyl. In some embodiments, R₁₁ is C₁-C₅ unsubstituted linear or branched alkyl. In other embodiments, R₁₁ is CH₃. In other embodiments, R₁₁ is CH₂CH₃. In other embodiments, R₁₁ is CH₂CH₂CH₃. In some embodiments, R₁₁ is isopropyl. In some embodiments, Ru is butyl. In some embodiments, R₁₁ is isobutyl. In some embodiments, R₁₁ is t-butyl. In some embodiments, R₁₁ is pentyl. In some embodiments, R₁₁ is isopentyl. In some embodiments, R₁₁ is neopentyl. In some embodiments, R₁₁ is benzyl. In some embodiments, R₁₁ is C₁-C₅ substituted linear or branched alkyl. In other embodiments, R₁₁ is CH₂—CH₂—O—CH₃. In other embodiments, R₁₁ is CH₂CF₃. In other embodiments, Ru is C₁-C₅ substituted or unsubstituted linear or branched haloalkyl. In other embodiments, Ru is C₁-C₅ linear or branched alkoxy. In other embodiments, Ru is O—CH₃. In other embodiments, Ru is R₂₀. In other embodiments, R₁₁ is C(O)R. In other embodiments, R₁₁ is S(O)₂R. In some embodiments, R₁₁ is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₁₀ and R₁₁ of formula I, II and/or I(a)-I(i) are joined to form a substituted or unsubstituted 3-8 membered heterocyclic ring. In other embodiments, R₁₀ and R₁₁ are joined to form a piperazine ring. In other embodiments, R₁₀ and R₁₁ are joined to form a piperidine ring. In some embodiments, substitutions include: F, Cl, Br, I, C₁-C₅ linear or branched alkyl, OH, alkoxy, OMe, amide, C(O)N(R)₂, C(O)-pyrrolidine, C(O)-piperidine, N(R)₂, NH(R₁₀), N(R₁₀)(R₁₁), N(CH₃)₂, NH₂, CF₃, aryl, phenyl, heteroaryl, substituted or unsubstituted C₃-C₈ cycloalkyl, cyclobutanol, substituted or unsubstituted 3-8 membered heterocyclic ring pyran, oxetane, piperidine, pyrazole, methyl-pyrrazole, triazole, imidazole, halophenyl, (benzyloxy)phenyl, CN, and NO₂; each represents a separate embodiment according to this invention.

In some embodiments, n of formula I, II, I(a)-I(b) and/or I(d)-I(i) is an integer between 0 and 4. In some embodiments, n of formula I(c) is an integer between 1 and 4. In some embodiments, n of formula I, II, I(a)-I(b) and/or I(d)-I(i) is 0. In some embodiments, n of formula I, II, and/or I(a)-I(i) is 1. In some embodiments, n of formula I, II, and/or I(a)-I(i) is 2. In some embodiments, n of formula I, II, and/or I(a)-I(i) is 3. In some embodiments, n of formula I, II, and/or I(a)-I(h) is 4. In some embodiments, n of formula I, II, and/or I(a)-I(h) is 1 or 2.

In some embodiments, A′ of formula I(f) is a 3-8 membered single or fused saturated, unsaturated or aromatic heterocyclic ring. In some embodiments, A′ is a 3-8 membered single heterocyclic ring. In some embodiments, A′ is a fused 4-10 membered heterocyclic ring. In some embodiments, A′ is a single aromatic 3-8 membered heterocyclic ring. In some embodiments, A′ is a fused aromatic 3-10 membered heterocyclic ring. In some embodiments, A′ is piperidine. In some embodiments, A′ is piperazine. In some embodiments, A′ is morpholine. In some embodiments, A′ is a pyridinyl. In other embodiments, A′ is 2-pyridinyl. In other embodiments, A′ is 3-pyridinyl. In other embodiments, A′ is 4-pyridinyl. In other embodiments, A′ is pyrimidine. In other embodiments, A′ is pyridazine. In other embodiments, A′ is pyrazine. In other embodiments, A′ is pyrazole. In other embodiments, A′ is benzothiazolyl. In other embodiments, A′ is benzimidazolyl. In other embodiments, A′ is quinolinyl. In other embodiments, A′ is isoquinolinyl. In other embodiments, A′ is indolyl. In other embodiments, A′ is indenyl. In other embodiments, A′ is benzofuran-2(3H)-one. In other embodiments, A′ is benzo[d][1,3]dioxole. In other embodiments, A′ is tetrahydrothiophenel, 1-dioxide. In other embodiments, A′ is thiazole. In other embodiments, A′ is benzimidazole. In others embodiment, A′ is piperidine. In other embodiments, A′ is imidazole. In other embodiments, A′ is thiophene. In other embodiments, A′ is isoquinoline. In other embodiments, A′ is indole. In other embodiments, A′ is 1,3-dihydroisobenzofuran. In other embodiments, A′ is benzofuran. In other embodiments, A′ is tetrahydro-2H-pyran. In other embodiments, A′ is isothiazolyl. In other embodiments, A′ is thiadiazolyl. In other embodiments, A′ is triazolyl. In other embodiments, A′ is thiazolyl. In other embodiments, A′ is oxazolyl. In other embodiments, A′ is isoxazolyl. In other embodiments, A′ is pyrrolyl. In other embodiments, A′ is furanyl. In other embodiments, A′ is oxadiazolyl. In other embodiments, A′ is oxadiazolyl. In other embodiments, A′ is 1,2,3-, 1,2,4-, 1,2,5- or 1,3,4-oxadiazolyl; each is a separate embodiment according to this invention. In other embodiments, A′ is tetrahydrofuranyl. In other embodiments, A′ is oxazolonyl. In other embodiments, A′ is oxazolidonyl. In other embodiments, A′ is thiazolonyl. In other embodiments, A′ is isothiazolinonyl. In other embodiments, A′ is isoxazolidinonyl. In other embodiments, A′ is imidazolidinonyl. In other embodiments, A′ is pyrazolonyl. In other embodiments, A′ is 2H-pyrrol-2-onyl. In other embodiments, A′ is furanonyl. In other embodiments, A′ is thiophenonyl. In other embodiments, A′ is thiane 1,1 dioxide. In other embodiments, A′ is triazolopyrimidine. In other embodiments, A′ is 3H-[1,2,3]triazolo[4,5-d]pyrimidine, 1H-[1,2,3]triazolo[4,5-d]pyrimidine, [1,2,4]triazolo[4,3-c]pyrimidine, [1,2,4]triazolo[4,3-a]pyrimidine, [1,2,3]triazolo[1,5-a]pyrimidine, [1,2,3]triazolo[1,5-c]pyrimidine, [1,2,4]triazolo[1,5-a]pyrimidine or [1,2,4]triazolo[1,5-c]pyrimidine; each is a separate embodiment according to this invention. In other embodiments, A′ is 6,7-dihydro-5H-pyrazolo[5,1-b][1,3]oxazine. In other embodiments, A′ is 1,2,3,4-tetrahydronaphthalene. In other embodiments, A′ is chroman. In other embodiments, A′ is isochroman. In other embodiments, A′ is 1,2,3,4-tetrahydroquinoline. In other embodiments, A′ is 1,2,3,4-tetrahydroisoquinoline. In other embodiments, A′ is 2,3-dihydro-1H-indene. In other embodiments, A′ is 2,3-dihydrobenzofuran. In other embodiments, A′ is 1,3-dihydroisobenzofuran. In other embodiments, A′ is isoindoline. In other embodiments, A′ is indoline.

In some embodiments, R₁₀₀ of formula I(g) is H, C₁-C₅ substituted or unsubstituted linear or branched alkyl (e.g., methyl), R₈—OH (e.g., (CH₂)₂—OH), —R₈—O—R₁₀ (e.g., (CH₂)₂O—CH₃), R₈—N(R₁₀)(R₁₁) (e.g., (e.g., (CH₂)₂-NH(CH₃), (CH₂)₂-NH₂), R₂₀, or a substituted or unsubstituted 3-8 membered heterocyclic ring (e.g., pyrrolidine, piperidine); each represents a separate embodiment according to this invention. In some embodiments, R₁₀₀ is H. In some embodiments, R₁₀₀ is C₁-C₅ substituted or unsubstituted linear or branched alkyl. In some embodiments, R₁₀₀ is C₁-C₅ unsubstituted linear or branched alkyl. In other embodiments, R₁₀₀ is CH₃. In other embodiments, R₁₀₀ is CH₂CH₃. In other embodiments, R₁₀₀ is CH₂CH₂CH₃. In some embodiments, R₁₀₀ is isopropyl. In some embodiments, R₁₀₀ is butyl. In some embodiments, R₁₀₀ is isobutyl. In some embodiments, R₁₀₀ is t-butyl. In some embodiments, R₁₀₀ is pentyl. In some embodiments, R₁₀₀ is isopentyl. In some embodiments, R₁₀₀ is neopentyl. In some embodiments, R₁₀₀ is benzyl. In some embodiments, R₁₀₀ is C₁-C₅ substituted linear or branched alkyl. In other embodiments, R₁₀₀ is CH₂—CH₂—O—CH₃. In other embodiments, R₁₀₀ is CH₂—CH₂—OH. In other embodiments, R₁₀₀ is R₈—OH. In other embodiments, R₁₀₀ is (CH₂)₂—OH. In other embodiments, R₁₀₀ is —R₈—O—R₁₀. In other embodiments, R₁₀₀ is (CH₂)₂—O—CH₃. In other embodiments, R₁₀₀ is R₈—N(R₁₀)(R₁₁). In other embodiments, R₁₀₀ is (CH₂)₂-NH(CH₃). In other embodiments, R₁₀₀ is (CH₂)₂-NH₂. In other embodiments, R₁₀₀ is R₂₀ as defined hereinabove. In other embodiments, R₁₀₀ is a substituted or unsubstituted 3-8 membered heterocyclic ring. In other embodiments, R₁₀₀ is pyrrolidine. In other embodiments, R₁₀₀ is piperidine. In other embodiments, R₁₀₀ is C₁-C₅ substituted or unsubstituted linear or branched haloalkyl. In other embodiments, R₁₀₀ is C₁-C₅ linear or branched alkoxy. In other embodiments, R₁₀₀ is O—CH₃. In other embodiments, R₁₀₀ is C(O)R. In other embodiments, R₁₀₀ is S(O)₂R. In some embodiments, R₁₀₀ is further substituted with at least one substitution selected from: F, Cl, Br, I, C₁-C₅ linear or branched alkyl, OH, alkoxy (e.g., OMe), amide (e.g., C(O)N(R)₂, C(O)-pyrrolidine, C(O)-piperidine, N(R)₂ NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

In some embodiments, R₁ of formula I(h) or A is H. In other embodiments R₁ is F. In other embodiments R₁ is CF₃. In other embodiments R₁ is Cl. In other embodiments R₁ is Br. In other embodiments R₁ is I. In other embodiments R₁ is OH. In other embodiments R₁ is SH. In other embodiments R₁ is substituted or unsubstituted C₁-C₅ alkyl. In other embodiments R₁ is C₁-C₅ linear or branched, or C₃-C₈ cyclic haloalkyl. In other embodiments R₁ is substituted or unsubstituted C₁-C₅ linear or branched, or C₃-C₈ cyclic alkoxy.

In some embodiments, R₂ of formula I(h) or A is H. In other embodiments R₂ is F. In other embodiments R₂ is CF₃. In other embodiments R₂ is Cl. In other embodiments R₂ is Br. In other embodiments R₂ is I. In other embodiments R₂ is OH. In other embodiments R₂ is SH. In other embodiments R₂ is substituted or unsubstituted C₁-C₅ alkyl. In other embodiments R₂ is C₁-C₅ linear or branched, or C₃-C₈ cyclic haloalkyl. In other embodiments R₂ is substituted or unsubstituted C₁-C₅ linear or branched, or C₃-C₈ cyclic alkoxy.

In some embodiments, R₁ and R₂ of formula I(h) or A are joined to form a 3-8 membered carbocyclic or heterocyclic ring. In other embodiments, R₁ and R₂ are joined to form a 3-8 membered carbocyclic ring. In some embodiments, the carbocyclic ring is cyclopropyl. In other embodiments, R₁ and R₂ are joined to form a 3-8 membered heterocyclic ring.

In some embodiments, R₃ of formula I(h) or A is H. In some embodiments, R₃ is methyl. In some embodiments, R₃ is substituted or unsubstituted C₁-C₅ alkyl. In some embodiments, the alkyl is methoxyethylene, methylaminoethylene, aminoethylene; each represents a separate embodiment according to this invention. In some embodiments, R₃ is —R₈—O—R₁₀. In some embodiments, R₃ is (CH₂)₂—O—CH₃. In some embodiments, R₃ is R₈—N(R₁₀)(R₁₁). In some embodiments, R₃ is (CH₂)₂—NH(CH₃)). In some embodiments, R₃ is substituted or unsubstituted C₃-C₈ cycloalkyl. In some embodiments, the cycloalkyl is cyclopropyl. In some embodiments, R₃ is substituted or unsubstituted 5-7 membered heterocyclic ring. In some embodiments, R₃ is pyrrolidine. In some embodiments, R₃ is methylpyrrolidine. In some embodiments, R₃ is piperidine. In some embodiments, R₃ is R₂₀ as defined hereinbelow.

In some embodiments, R₄ of formula I(h) or A is H. In some embodiments, R₄ is methyl. In some embodiments, R₄ is substituted or unsubstituted C₁-C₅ alkyl. In some embodiments, the alkyl is methoxyethylene, methylaminoethylene, aminoethylene; each represents a separate embodiment according to this invention. In some embodiments, R₄ is —R₈—O—R₁₀. In some embodiments, R₄ is (CH₂)₂—O—CH₃. In some embodiments, R₄ is R₈—N(R₁₀)(R₁₁). In some embodiments, R₄ is (CH₂)₂—NH(CH₃)). In some embodiments, R₄ is substituted or unsubstituted C₃-C₈ cycloalkyl. In some embodiments, the cycloalkyl is cyclopropyl. In some embodiments, R₄ is substituted or unsubstituted 5-7 membered heterocyclic ring. In some embodiments, R₄ is pyrrolidine. In some embodiments, R₄ is methylpyrrolidine. In some embodiments, R₄ is piperidine. In some embodiments, R₄ is R₂₀ as defined hereinbelow.

In some embodiments, R₂ and R₄ of formula I(h) or A are joined to form Ring F as defined hereinbelow. In some embodiments, R₂ and R₄ are joined to form a substituted or unsubstituted, saturated or unsaturated, 4-8 membered heterocyclic ring. In some embodiments, R₂ and R₄ are joined to form a substituted or unsubstituted, unsaturated, 4-8 membered heterocyclic ring. In some embodiments, R₂ and R₄ are joined to form pyrrolidine, 1-methylpyrrolidine, pyrrolidin-2-one, pyridine, piperidine, imidazole, pyrimidine, triazole, oxadiazole, pyrazole; each represents a separate embodiment according to this invention. In some embodiments, if Ring F is aromatic, then R₁ is absent. In some embodiments, if Ring F is aromatic, then R₃ is absent. In some embodiments, if Ring F is aromatic, then R₁ and/or R₃ are absent.

In some embodiments, R₃ and R₄ of formula I(h) or A are joined to form a 3-8 membered heterocyclic ring. In some embodiments, the heterocyclic ring is pyrrolidine, pyrrolidone, 2-oxopyrrolidine, piperidine, morpholine, piperazine, imidazole; each represents a separate embodiment according to this invention.

In some embodiments, Ring F of formula I(h) is absent. In some embodiments, Ring F is a substituted or unsubstituted, saturated or unsaturated, 4-8 membered heterocyclic ring. In some embodiments, Ring F is a substituted, saturated, 4-8 membered heterocyclic ring. In some embodiments, Ring F is a substituted unsaturated, 4-8 membered heterocyclic ring. In some embodiments, Ring F is an unsubstituted, saturated, 4-8 membered heterocyclic ring. In some embodiments, Ring F is an unsubstituted, unsaturated, 4-8 membered heterocyclic ring. In some embodiments, Ring F is pyrrolidine. In some embodiments, Ring F is pyrrolidine-2-one. In some embodiments, Ring F is piperidine. In some embodiments, Ring F is piperazine. In some embodiments, Ring F is morpholine. In some embodiments, Ring F is a pyridinyl. In other embodiments, Ring F is 2-pyridinyl. In other embodiments, Ring F is pyrimidine. In other embodiments, Ring F is imidazole. In other embodiments, Ring F is pyridazine. In other embodiments, Ring F is pyrazine. In other embodiments, Ring F is pyrazole. In other embodiments, Ring F is thiazole. In other embodiments, Ring F is isothiazolyl. In other embodiments, Ring F is thiadiazolyl. In other embodiments, Ring F is triazolyl. In other embodiments, Ring F is thiazolyl. In other embodiments, Ring F is oxazolyl. In other embodiments, Ring F is isoxazolyl. In other embodiments, Ring F is pyrrolyl. In other embodiments, Ring F is oxadiazolyl. In other embodiments, Ring F is 1,2,3-, 1,2,4-, 1,2,5- or 1,3,4-oxadiazolyl; each is a separate embodiment according to this invention. In other embodiments, Ring F is oxazolonyl. In other embodiments, Ring F is oxazolidonyl. In other embodiments, Ring F is thiazolonyl. In other embodiments, Ring F is isothiazolinonyl. In other embodiments, Ring F is isoxazolidinonyl. In other embodiments, Ring F is imidazolidinonyl. In other embodiments, Ring F is pyrazolonyl. In other embodiments, Ring F is 2H-pyrrol-2-onyl. In other embodiments, Ring F is triazolopyrimidine. In other embodiments, Ring F is 3H-[1,2,3]triazolo[4,5-d]pyrimidine, 1H-[1,2,3]triazolo[4,5-d]pyrimidine, [1,2,4]triazolo[4,3-c]pyrimidine, [1,2,4]triazolo[4,3-a]pyrimidine, [1,2,3]triazolo[1,5-a]pyrimidine, [1,2,3]triazolo[1,5-c]pyrimidine, [1,2,4]triazolo[,5-a]pyrimidine or [1,2,4]triazolo[1,5-c]pyrimidine; each is a separate embodiment according to this invention. In other embodiments, Ring F is 6,7-dihydro-5H-pyrazolo[5,1-b][1,3]oxazine.

In various embodiments, this invention is directed to the compounds presented in Table 1, pharmaceutical compositions and/or method of use thereof, each represents a separate embodiment according to this invention:

TABLE 1

| Compound | | |
|---|---|---|
| No. | Structure | Compound Name |
| 101 | 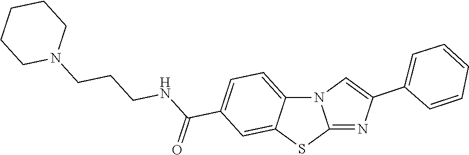 [SMILES: O=C(NCCCN1CCCCC1)c1ccc2c(c1)sc1nc(-c3ccccc3)cn12] | 2-phenyl-N-(3-(piperidin-1- yl)propyl)benzo[d]imidazo[2, 1-b]thiazole-7-carboxamide |
| 102 | 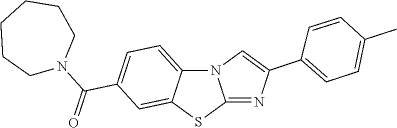 [SMILES: Cc1ccc(-c2cn3c(n2)sc2cc(C(=O)N4CCCCCC4)ccc23)cc1] | Azepan-1-yl(2-(p- tolyl)benzo[d]imidazo[2,1- b]thiazol-7-Amethanone |
| 103 | 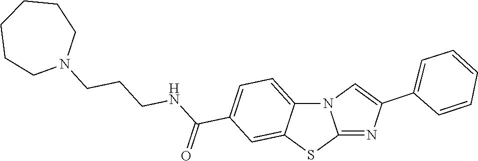 [SMILES: O=C(NCCCN1CCCCCC1)c1ccc2c(c1)sc1nc(-c3ccccc3)cn12] | N-(3-(azepan-1-yl)propyl)-2- phenylbenzo[d]imidazo[2,1- b]thiazole-7-carboxamide |
| 104 | 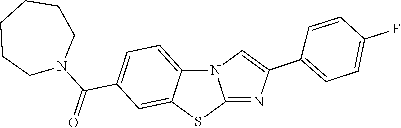 [SMILES: O=C(c1ccc2c(c1)sc1nc(-c3ccc(F)cc3)cn12)N1CCCCCC1] | azepan-1-yl(2-(4- fluorophenyl)benzo[d] imidazo[2,1-b]thiazol-7- yl)methanone |
| 105 | 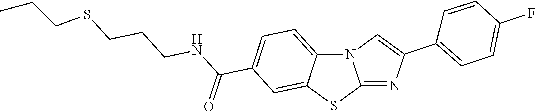 [SMILES: CCCSCCCNC(=O)c1ccc2c(c1)sc1nc(-c3ccc(F)cc3)cn12] | 2-(4-fluorophenyl)-N-(3- (propylthio)propyl)benzo[d] imidazo[2,1-b]thiazole-7- carboxamide |
| 106 | 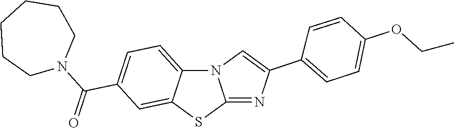 [SMILES: CCOc1ccc(-c2cn3c(n2)sc2cc(C(=O)N4CCCCCC4)ccc23)cc1] | azepan-1-yl(2-(4- ethoxyphenyl)benzo[d] imidazo[2,1-b]thiazol-7- yl)methanone |
| 107 | 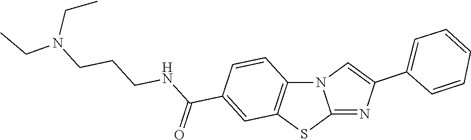 [SMILES: CCN(CC)CCCNC(=O)c1ccc2c(c1)sc1nc(-c3ccccc3)cn12] | N-(3-(diethylamino)propyl)- 2-phenylbenzo[d]imidazo [2,1-b]thiazole-7- carboxamide |
| 108 | 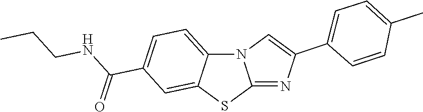 [SMILES: CCCNC(=O)c1ccc2c(c1)sc1nc(-c3ccc(C)cc3)cn12] | N-propyl-2-(p- tolyl)benzo[d]imidazo[2,1- b]thiazole-7-carboxamide |
| 109 | 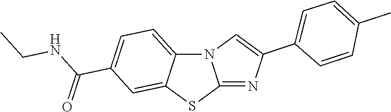 [SMILES: CCNC(=O)c1ccc2c(c1)sc1nc(-c3ccc(C)cc3)cn12] | N-ethyl-2-(p- tolyl)benzo[d]imidazo[2,1- b]thiazole-7-carboxamide |
| 110 | 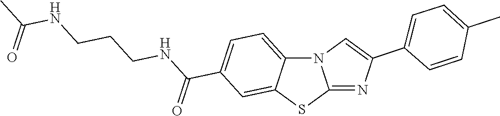 [SMILES: CC(=O)NCCCNC(=O)c1ccc2c(c1)sc1nc(-c3ccc(C)cc3)cn12] | N-(3-acetamidopropyl)-2- (p-tolyl)benzo[d]imidazo[2,1- b]thiazole-7-carboxamide |
| 111 | 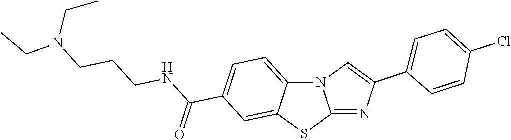 [SMILES: CCN(CC)CCCNC(=O)c1ccc2c(c1)sc1nc(-c3ccc(Cl)cc3)cn12] | 2-(4-chlorophenyl)-N-(3- (diethylamino)propyl)benzo [d]imidazo[2,1-b]thiazole-7- carboxamide |
| 114 | 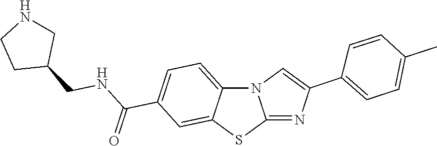 [SMILES: Cc1ccc(-c2cn3c(n2)sc2cc(C(=O)NC[C@H]4CCNC4)ccc23)cc1] | (S)-N-(pyrrolidin-3- ylmethyl)-2-(p- tolyl)benzo[d]imidazo[2,1- b]thiazole-7-carboxamide formate |
| 115 | 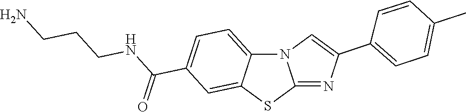 [SMILES: Cc1ccc(-c2cn3c(n2)sc2cc(C(=O)NCCCN)ccc23)cc1] | N-(3-aminopropyl)-2-(p- tolyl)benzo[d]imidazo[2,1- b]thiazole-7-carboxamide |
| 116 | 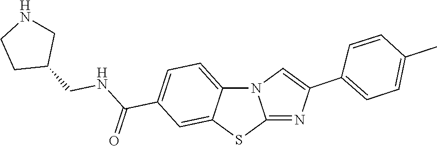 [SMILES: Cc1ccc(-c2cn3c(n2)sc2cc(C(=O)NC[C@@H]4CCNC4)ccc23)cc1] | (R)-N-(pyrrolidin-3- ylmethyl)-2-(p- tolyl)benzo[d]imidazo [2,1-b]thiazole-7- carboxamide formate |
| 117 | 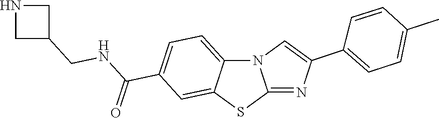 [SMILES: Cc1ccc(-c2cn3c(n2)sc2cc(C(=O)NCC4CNC4)ccc23)cc1] | N-(azetidin-3-ylmethyl)-2- (p-tolyl)benzo[d]imidazo [2,1-b]thiazole-7- carboxamide |
| 118 | 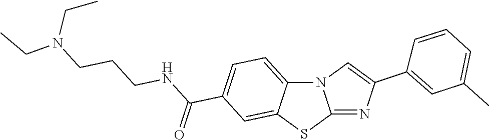 [SMILES: CCN(CC)CCCNC(=O)c1ccc2c(c1)sc1nc(-c3cccc(C)c3)cn12] | N-(3-(diethylamino)propyl)- 2-(m-tolyl)benzo[d]imidazo [2,1-b]thiazole-7-carboxamide |
| 119 | 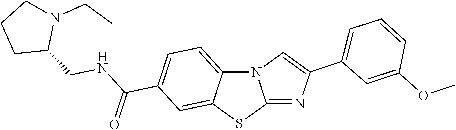 [SMILES: CCN1CCC[C@H]1CNC(=O)c1ccc2c(c1)sc1nc(-c3cccc(OC)c3)cn12] | (S)-N-((1-ethylpyrrolidin-2- yl)methyl)-2-(3- methoxyphenyl)benzo[d] imidazo[2,1-b]thiazole-7- carboxamide |
| 122 | 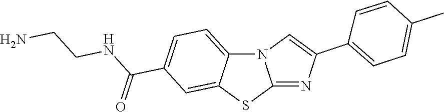 [SMILES: Cc1ccc(-c2cn3c(n2)sc2cc(C(=O)NCCN)ccc23)cc1] | N-(2-aminoethyl)-2-(p- tolyl)benzo[d]imidazo[2,1- b]thiazole-7-carboxamide |
| 123 | 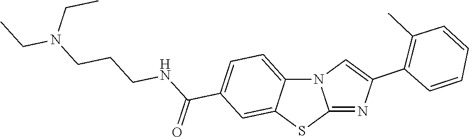 [SMILES: CCN(CC)CCCNC(=O)c1ccc2c(c1)sc1nc(-c3ccccc3C)cn12] | N-(3-(diethylamino)propyl)- 2-(o-tolyl)benzo[d]imidazo [2,1-b]thiazole-7- carboxamide |
| 124 | 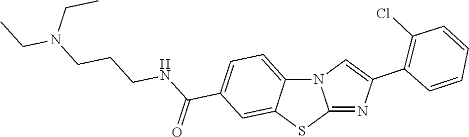 [SMILES: CCN(CC)CCCNC(=O)c1ccc2c(c1)sc1nc(-c3ccccc3Cl)cn12] | 2-(2-ehlorophenyl)-N-(3- (diethylamino)propyl)benzo [d]imidazo[2,1-b]thiazole-7- carboxamide |
| 125 | 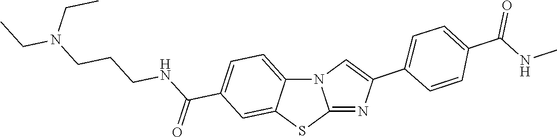 [SMILES: CCN(CC)CCCNC(=O)c1ccc2c(c1)sc1nc(-c3ccc(C(=O)NC)cc3)cn12] | N-(3-(diethylamino)propyl)- 2-(4- (methylcarbamoyl)phenyl) benzo[d]imidazo[2,1- b]thiazole-7-carboxamide |
| 126 | 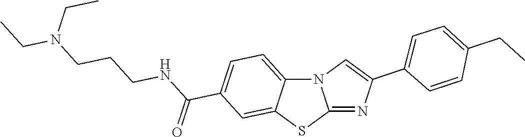 [SMILES: CCc1ccc(-c2cn3c(n2)sc2cc(C(=O)NCCCN(CC)CC)ccc23)cc1] | N-(3-(diethylamino)propyl)- 2-(4- ethylphenyl)benzo[d] imidazo[2,1-b]thiazole-7- carboxamide |
| 127 | 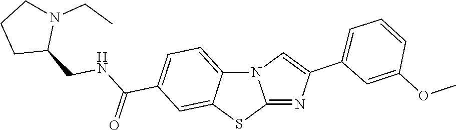 [SMILES: CCN1CCC[C@@H]1CNC(=O)c1ccc2c(c1)sc1nc(-c3cccc(OC)c3)cn12] | (R)-N-((1-ethylpyrrolidin-2- yl)methyl)-2-(3- methoxyphenyl)benzo[d] imidazo[2,1-b]thiazole-7- carboxamide |
| 129 | 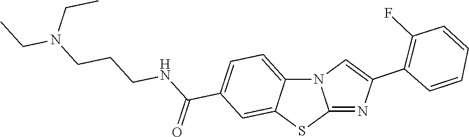 [SMILES: CCN(CC)CCCNC(=O)c1ccc2c(c1)sc1nc(-c3ccccc3F)cn12] | N-(3-(diethylamino)propyl)- 2-(2-fluorophenyl)benzo[d] imidazo[2,1-b]thiazole-7- carboxamide |
| 130 | 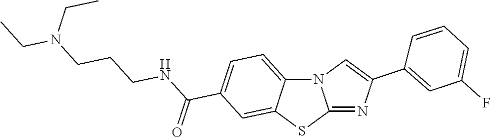 [SMILES: CCN(CC)CCCNC(=O)c1ccc2c(c1)sc1nc(-c3cccc(F)c3)cn12] | N-(3-(diethylamino)propyl)- 2-(3- fluorophenyl)benzo[d] imidazo[2,1-b]thiazole-7- carboxamide |
| 131 | 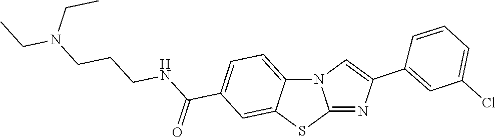 [SMILES: CCN(CC)CCCNC(=O)c1ccc2c(c1)sc1nc(-c3cccc(Cl)c3)cn12] | 2-(3-chlorophenyl)-N-(3- (diethylamino)propyl)benzo [d]imidazo[2,1-b]thiazole-7- carboxamide |
| 132 | 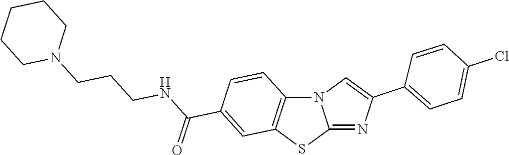 [SMILES: O=C(NCCCN1CCCCC1)c1ccc2c(c1)sc1nc(-c3ccc(Cl)cc3)cn12] | 2-(4-chlorophenyl)-N-(3- (piperidin-1-yl)propyl) benzo[d]imidazo[2,1- b]thiazole-7-carboxamide |
| 133 | 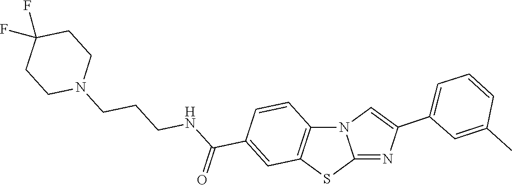 [SMILES: Cc1cccc(-c2cn3c(n2)sc2cc(C(=O)NCCCN4CCC(F)(F)CC4)ccc23)c1] | N-(3-(4,4-difluoropiperidin- 1-yl)propyl)-2-(m- tolyl)benzo[d]imidazo[2,1- b]thiazole-7-carboxamide |
| 134 | 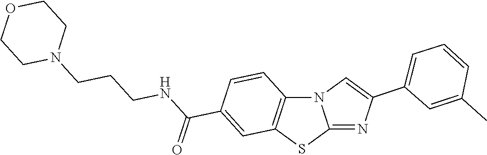 [SMILES: Cc1cccc(-c2cn3c(n2)sc2cc(C(=O)NCCCN4CCOCC4)ccc23)c1] | N-(3-morpholinopropyl)-2- (m-tolyl)benzo[d]imidazo[2,1- b]thiazole-7-carboxamide |
| 135 | 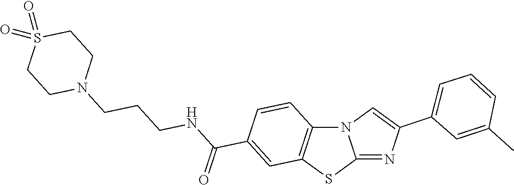 [SMILES: Cc1cccc(-c2cn3c(n2)sc2cc(C(=O)NCCCN4CCS(=O)(=O)CC4)ccc23)c1] | N-(3-(1,1-dioxidothiomorpholino) propyl)-2-(m- tolyl)benzo[d]imidazo[2,1- b]thiazole-7-carboxamide |
| 136 | 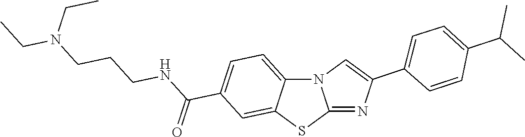 [SMILES: CCN(CC)CCCNC(=O)c1ccc2c(c1)sc1nc(-c3ccc(C(C)C)cc3)cn12] | N-(3-(diethylamino)propyl)- 2-(4- isopropylphenyl)benzo[d] imidazo[2,1-b]thiazole-7- carboxamide formate |
| 137 | 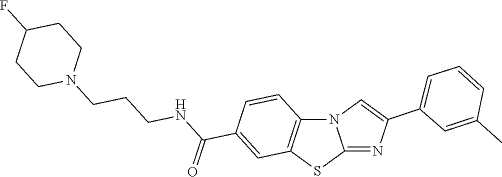 [SMILES: Cc1cccc(-c2cn3c(n2)sc2cc(C(=O)NCCCN4CCC(F)CC4)ccc23)c1] | N-(3-(4-fluoropiperidin-1- yl)propyl)-2-(m- tolyl)benzo[d]imidazo[2,1- b]thiazole-7-carboxamide |
| 138 | 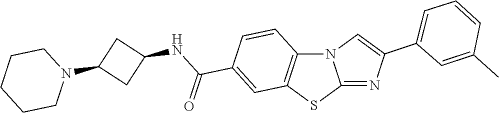 [SMILES: Cc1cccc(-c2cn3c(n2)sc2cc(C(=O)N[C@H]4C[C@@H](N5CCCCC5)C4)ccc23)c1] | N-((1s,3s)-3-(piperidin-1- yl)cyclobutyl)-2-(m- tolyl)benzo[d]imidazo[2,1- b]thiazole-7-carboxamide |
| 139 | 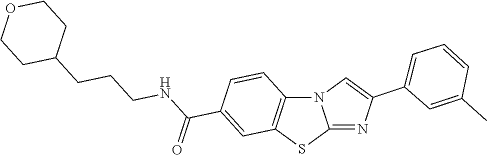 [SMILES: Cc1cccc(-c2cn3c(n2)sc2cc(C(=O)NCCCC4CCOCC4)ccc23)c1] | N-(3-(tetrahydro-2H-pyran- 4-yl)propyl)-2-(m- tolyl)benzo[d]imidazo[2,1- b]thiazole-7-carboxamide |
| 140 | 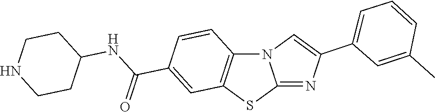 [SMILES: Cc1cccc(-c2cn3c(n2)sc2cc(C(=O)NC4CCNCC4)ccc23)c1] | N-(piperidin-4-yl)-2-(m- tolyl)benzo[d]imidazo[2,1- b]thiazole-7-carboxamide |
| 141 | 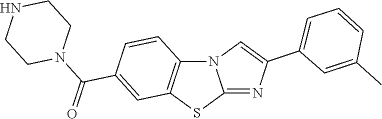 [SMILES: Cc1cccc(-c2cn3c(n2)sc2cc(C(=O)N4CCNCC4)ccc23)c1] | piperazin-1-yl(2-(m- tolyl)benzo[d]imidazo[2,1- b]thiazol-7-yl)methanone |
| 142 | 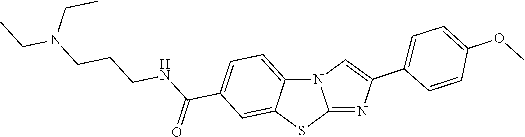 [SMILES: CCN(CC)CCCNC(=O)c1ccc2c(c1)sc1nc(-c3ccc(OC)cc3)cn12] | N-(3-(diethylamino)propyl)- 2-(4- methoxyphenyl)benzo[d] imidazo[2,1-b]thiazole-7- carboxamide |
| 143 | 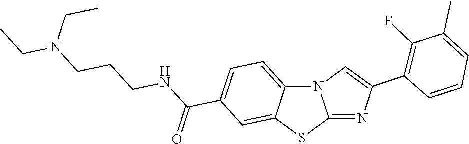 [SMILES: CCN(CC)CCCNC(=O)c1ccc2c(c1)sc1nc(-c3cccc(C)c3F)cn12] | N-(3-(diethylamino)propyl)- 2-(2-fluoro-3- methylphenyl)benzo[d] imidazo[2,1-b]thiazole-7- carboxamide |
| 144 | 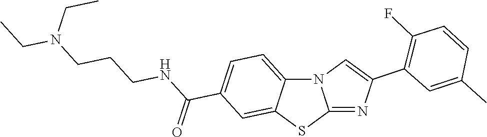 [SMILES: CCN(CC)CCCNC(=O)c1ccc2c(c1)sc1nc(-c3cc(C)ccc3F)cn12] | N-(3-(diethylamino)propyl)- 2-(2-fluoro-5- methylphenyl)benzo[d] imidazo[2,1-b]thiazole-7- carboxamide |
| 145 | 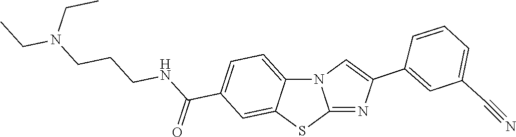 [SMILES: CCN(CC)CCCNC(=O)c1ccc2c(c1)sc1nc(-c3cccc(C#N)c3)cn12] | 2-(3-cyanophenyl)-N-(3- (diethylamino)propyl)benzo [d]imidazo[2,1-b]thiazole-7- carboxamide |
| 149 | 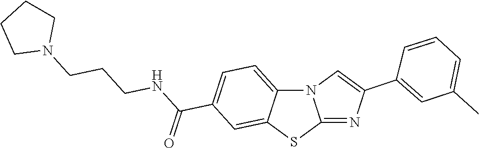 [SMILES: Cc1cccc(-c2cn3c(n2)sc2cc(C(=O)NCCCN4CCCC4)ccc23)c1] | N-(3-(pyrrolidin-1- yl)propyl)-2-(m- tolyl)benzo[d]imidazo[2,1- b]thiazole-7-carboxamide |
| 150 | 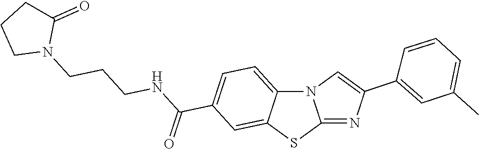 [SMILES: Cc1cccc(-c2cn3c(n2)sc2cc(C(=O)NCCCN4CCCC4=O)ccc23)c1] | N-(3-(2-oxopyrrolidin-1- yl)propyl)-2-(m- tolyl)benzo[d]imidazo[2,1- b]thiazole-7-carboxamide |
| 151 | 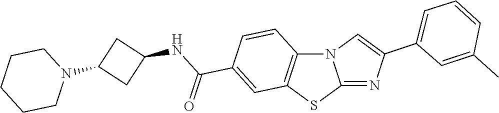 [SMILES: Cc1cccc(-c2cn3c(n2)sc2cc(C(=O)N[C@H]4C[C@H](N5CCCCC5)C4)ccc23)c1] | N-((1r,3r)-3-(piperidin-1- yl)cyclobutyl)-2-(m- tolyl)benzo[d]imidazo[2,1- b]thiazole-7-carboxamide |
| 152 | 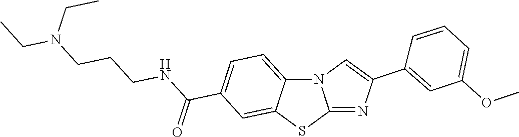 [SMILES: CCN(CC)CCCNC(=O)c1ccc2c(c1)sc1nc(-c3cccc(OC)c3)cn12] | N-(3-(diethylamino)propyl)- 2-(3- methoxyphenyl)benzo[d] imidazo[2,1-b]thiazole-7- carboxamide |
| 153 | 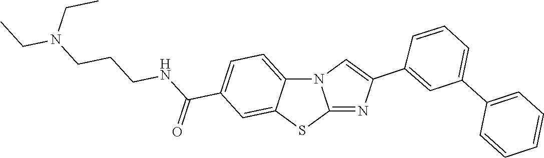 [SMILES: CCN(CC)CCCNC(=O)c1ccc2c(c1)sc1nc(-c3cccc(-c4ccccc4)c3)cn12] | 2-([1,1′-biphenyl]-3-yl)-N- (3-(diethylamino)propyl)benzo [d]imidazo[2,1-b]thiazole-7- carboxamide |
| 154 | 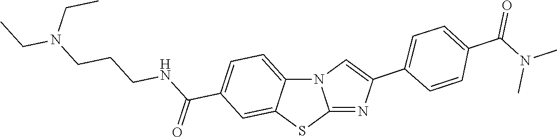 [SMILES: CCN(CC)CCCNC(=O)c1ccc2c(c1)sc1nc(-c3ccc(C(=O)N(C)C)cc3)cn12] | N-(3-(diethylamino)propyl)- 2-(4- (dimethylcarbamoyl)phenyl) benzo[d]imidazo[2,1- b]thiazole-7-carboxamide formate |
| 155 | 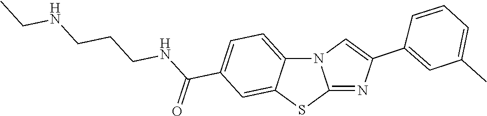 [SMILES: CCNCCCNC(=O)c1ccc2c(c1)sc1nc(-c3cccc(C)c3)cn12] | N-(3-(ethylamino)propyl)-2- tolyl)benzo[d]imidazo[2,1- b]thiazole-7-carboxamide |
| 156 | 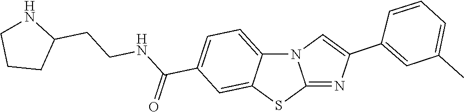 [SMILES: Cc1cccc(-c2cn3c(n2)sc2cc(C(=O)NCCC4CCCN4)ccc23)c1] | N-(2-(pyrrolidin-2-yl)ethyl)- 2-(m- tolyl)benzo[d]imidazo[2,1- b]thiazole-7-carboxamide |
| 157 | 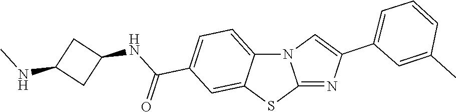 | N-((1s,3s)-3- (methylamino)cyclobutyl)-2- (m-tolyl)benzo[d]imidazo[2,1- b]thiazole-7-carboxamide formate |
| 158 | 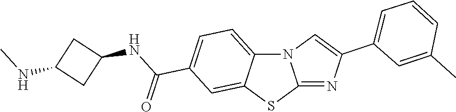 | N-((1r,3r)-3- (methylamino)cyclobutyl)-2- (m- tolyl)benzo[d]imidazo[2,1- b]thiazole-7-carboxamide formate |
| 159 | 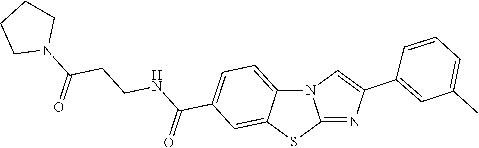 [SMILES: Cc1cccc(-c2cn3c(n2)sc2cc(C(=O)NCCC(=O)N4CCCC4)ccc23)c1] | N-(3-oxo-3-(pyrrolidin-1- yl)propyl)-2-(m- tolyl)benzo[d]imidazo[2,1- b]thiazole-7-carboxamide |
| 160 | 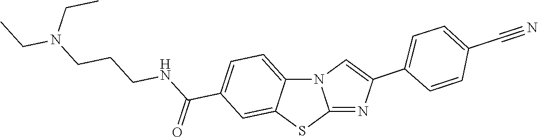 [SMILES: CCN(CC)CCCNC(=O)c1ccc2c(c1)sc1nc(-c3ccc(C#N)cc3)cn12] | 2-(4-cyanophenyl)-N-(3- (diethylamino)propyl)benzo [d]imidazo[2,1-b]thiazole-7- carboxamide |
| 161 | 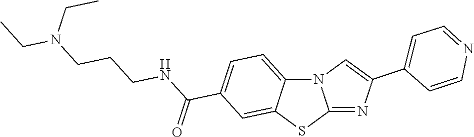 [SMILES: CCN(CC)CCCNC(=O)c1ccc2c(c1)sc1nc(-c3ccncc3)cn12] | N-(3-(diethylamino)propyl)- 2-(pyridin-4- yl)benzo[d]imidazo[2,1- b]thiazole-7-carboxamide |
| 162 | 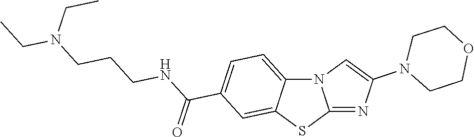 [SMILES: CCN(CC)CCCNC(=O)c1ccc2c(c1)sc1nc(N3CCOCC3)cn12] | N-(3-(diethylamino)propyl)- 2- morpholinobenzo[d]imidazo [2,1-b]thiazole-7- carboxamide |
| 163 | 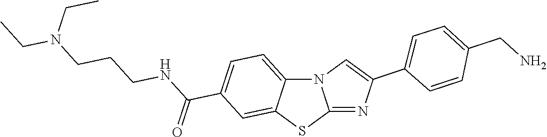 [SMILES: CCN(CC)CCCNC(=O)c1ccc2c(c1)sc1nc(-c3ccc(CN)cc3)cn12] | 2-(4-(aminomethyl)phenyl)- N-(3- (diethylamino)propyl)benzo [d]imidazo[2,1-b]thiazole-7- carboxamide |
| 164 | 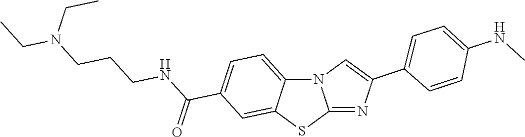 [SMILES: CCN(CC)CCCNC(=O)c1ccc2c(c1)sc1nc(-c3ccc(NC)cc3)cn12] | N-(3-(diethylamino)propyl)- 2-(4- (methylamino)phenyl)benzo [d]imidazo[2,1-b]thiazole-7- carboxamide |
| 165 | 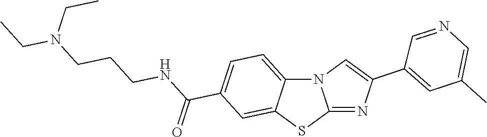 [SMILES: CCN(CC)CCCNC(=O)c1ccc2c(c1)sc1nc(-c3cncc(C)c3)cn12] | N-(3-(diethylamino)propyl)- 2-(5-methylpyridin-3- yl)benzo[d]imidazo[2,1- b]thiazole-7-carboxamide |
| 166 | 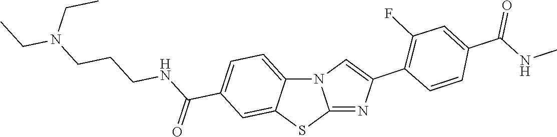 [SMILES: CCN(CC)CCCNC(=O)c1ccc2c(c1)sc1nc(-c3ccc(C(=O)NC)cc3F)cn12] | N-(3-(diethylamino)propyl)- 2-(2-fluoro-4- (methylcarbamoyl)phenyl) benzo[d]imidazo[2,1- b]thiazole-7-carboxamide |
| 167 | 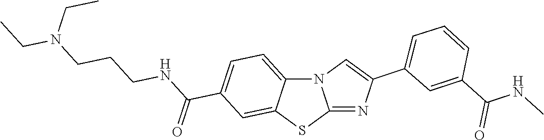 [SMILES: CCN(CC)CCCNC(=O)c1ccc2c(c1)sc1nc(-c3cccc(C(=O)NC)c3)cn12] | N-(3-(diethylamino)propyl)- 2-(3- (methylcarbamoyl)phenyl) benzo[d]imidazo[2,1- b]thiazole-7-carboxamide |
| 168 | 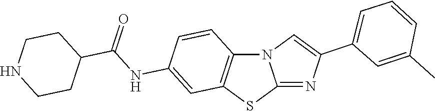 [SMILES: Cc1cccc(-c2cn3c(n2)sc2cc(NC(=O)C4CCNCC4)ccc23)c1] | N-(2-(m- tolyl)benzo[d]imidazo[2,1- b]thiazol-7-yl)piperidine-4- carboxamide |
| 169 | 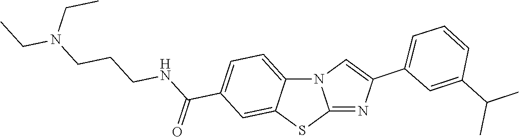 [SMILES: CCN(CC)CCCNC(=O)c1ccc2c(c1)sc1nc(-c3cccc(C(C)C)c3)cn12] | N-(3-(diethylamino)propyl)- 2-(3- isopropylphenyl)benzo[d] imidazo[2,1-b]thiazole-7- carboxamide |
| 170 | 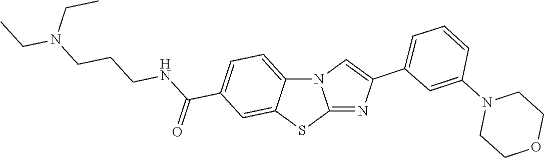 [SMILES: CCN(CC)CCCNC(=O)c1ccc2c(c1)sc1nc(-c3cccc(N4CCOCC4)c3)cn12] | N-(3-(diethylamino)propyl)- 2-(3- morpholinophenyl)benzo[d] imidazo[2,1-b]thiazole-7- carboxamide |
| 171 | 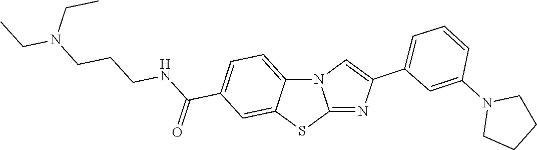 [SMILES: CCN(CC)CCCNC(=O)c1ccc2c(c1)sc1nc(-c3cccc(N4CCCC4)c3)cn12] | N-(3-(diethylamino)propyl)- 2-(3-(pyrrolidin-1- yl)phenyl)benzo[d]imidazo [2,1-b]thiazole-7-carboxamide |
| 172 | 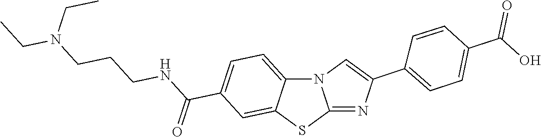 [SMILES: CCN(CC)CCCNC(=O)c1ccc2c(c1)sc1nc(-c3ccc(C(=O)O)cc3)cn12] | 4-(7-((3- (diethylamino)propyl) carbamoyl)benzo[d]imidazo [2,1-b]thiazol-2-yl)benzoic acid |
| 173 | 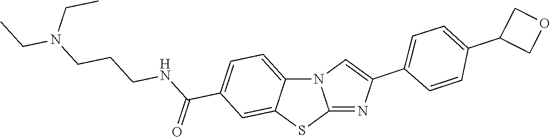 [SMILES: CCN(CC)CCCNC(=O)c1ccc2c(c1)sc1nc(-c3ccc(C4COC4)cc3)cn12] | N-(3-(diethylamino)propyl)- 2-(4-(oxetan-3- yl)phenyl)benzo[d]imidazo [2,1-b]thiazole-7-carboxamide |
| 174 | 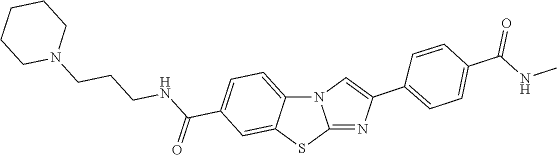 [SMILES: CNC(=O)c1ccc(-c2cn3c(n2)sc2cc(C(=O)NCCCN4CCCCC4)ccc23)cc1] | 2-(4- (methylcarbamoyl)phenyl)- N-(3-(piperidin-1- yl)propyl)benzo[d]imidazo [2,1-b]thiazole-7-carboxamide |
| 175 | 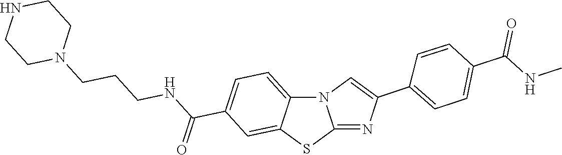 [SMILES: CNC(=O)c1ccc(-c2cn3c(n2)sc2cc(C(=O)NCCCN4CCNCC4)ccc23)cc1] | 2-(4- (methylcarbamoyl)phenyl)- N-(3-(piperazin-1- yl)propyl)benzo[d]imidazo [2,1-b]thiazole-7-carboxamide |
| 176 | 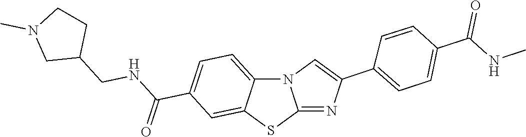 | 2-(4- (methylcarbamoyl)phenyl)- N-((1-methylpyrrolidin-3- yl)methyl)benzo[d]imidazo [2,1-b]thiazole-7-carboxamide |
| 177 | 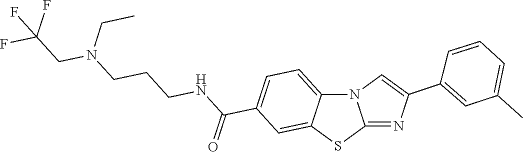 [SMILES: CCN(CCCNC(=O)c1ccc2c(c1)sc1nc(-c3cccc(C)c3)cn12)CC(F)(F)F] | N-(3-(ethyl(2,2,2- trifluoroethyl)amino)propyl)- 2-(m- tolyl)benzo[d]imidazo[2,1- b]thiazole-7-carboxamide |
| 178 | 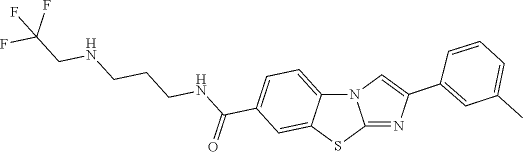 [SMILES: Cc1cccc(-c2cn3c(n2)sc2cc(C(=O)NCCCNCC(F)(F)F)ccc23)c1] | 2-(m-tolyl)-N-(3-((2,2,2- trifluoroethyl)amino)propyl) benzo[d]imidazo[2,1- b]thiazole-7-carboxamide |
| 179 | 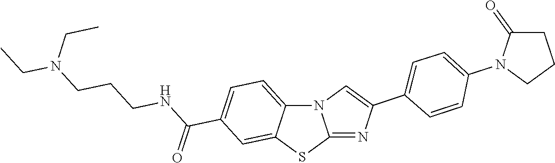 [SMILES: CCN(CC)CCCNC(=O)c1ccc2c(c1)sc1nc(-c3ccc(N4CCCC4=O)cc3)cn12] | N-(3-(diethylamino)propyl)- 2-(4-(2-oxopyrrolidin-1- yl)phenyl)benzo[d]imidazo [2,1-b]thiazole-7-carboxamide |
| 180 | 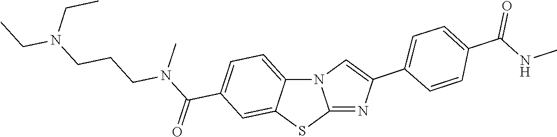 [SMILES: CCN(CC)CCCN(C)C(=O)c1ccc2c(c1)sc1nc(-c3ccc(C(=O)NC)cc3)cn12] | N-(3-(diethylamino)propyl)- N-methyl-2-(4- (methylcarbamoyl)phenyl) benzo[d]imidazo[2,1- b]thiazole-7-carboxamide |
| 181 | 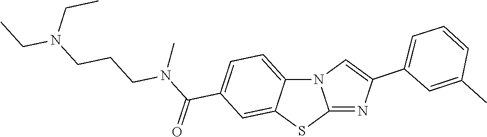 [SMILES: CCN(CC)CCCN(C)C(=O)c1ccc2c(c1)sc1nc(-c3cccc(C)c3)cn12] | N-(3-(diethylamino)propyl)- N-methyl-2-(m- tolyl)benzo[d]imidazo[2,1- b]thiazole-7-carboxamide |
| 182 | 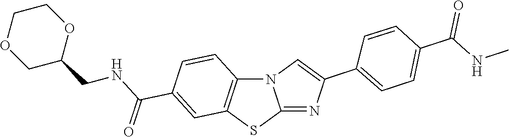 [SMILES: CNC(=O)c1ccc(-c2cn3c(n2)sc2cc(C(=O)NC[C@H]4COCCO4)ccc23)cc1] | (S)-N-((1,4-dioxan-2- yl)methyl)-2-(4- (methylcarbamoyl)phenyl) benzo[d]imidazo[2,1- b]thiazole-7-carboxamide |
| 183 | 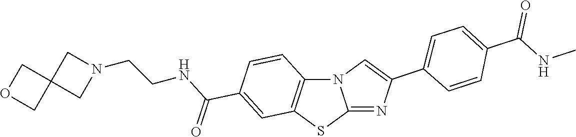 [SMILES: CNC(=O)c1ccc(-c2cn3c(n2)sc2cc(C(=O)NCCN4CC5(COC5)C4)ccc23)cc1] | N-(2-(2-oxa-6- azaspiro[3.3]heptan-6- yl)ethyl)-2-(4- (methylcarbamoyl)phenyl) benzo[d]imidazo[2,1- b]thiazole-7-carboxamide |
| 184 | 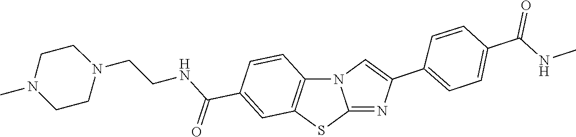 | 2-(4- (methylcarbamoyl)phenyl)- N-(2-(4-methylpiperazin-1- yl)ethyl)benzo[d]imidazo [2,1-b]thiazole-7-carboxamide |
| 185 | 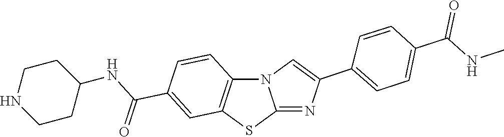 [SMILES: CNC(=O)c1ccc(-c2cn3c(n2)sc2cc(C(=O)NC4CCNCC4)ccc23)cc1] | 2-(4- (methylcarbamoyl)phenyl)- N-(piperidin-4- yl)benzo[d]imidazo[2,1- b]thiazole-7-carboxamide |
| 186 | 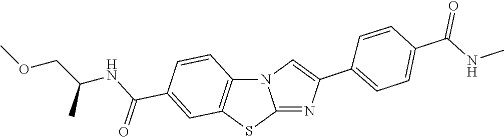 | (S)-N-(1-methoxypropan-2- (methylcarbamoyl)phenyl) benzo[d]imidazo[2,1- b]thiazole-7-carboxamide |
| 187 | 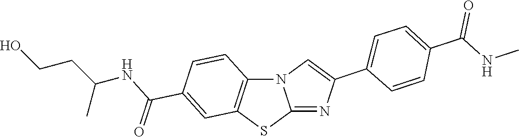 [SMILES: CNC(=O)c1ccc(-c2cn3c(n2)sc2cc(C(=O)NC(C)CCO)ccc23)cc1] | N-(4-hydroxybutan-2-yl)-2- (4- (methylcarbamoyl)phenyl) benzo[d]imidazo[2,1- b]thiazole-7-carboxamide |
| 188 | 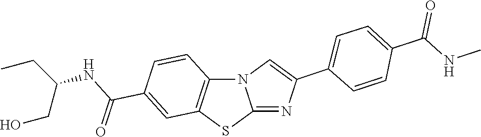 [SMILES: CC[C@@H](CO)NC(=O)c1ccc2c(c1)sc1nc(-c3ccc(C(=O)NC)cc3)cn12] | (S)-N-(1-hydroxybutan-2-yl)- 2-(4- (methylcarbamoyl)phenyl) benzo[d]imidazo[2,1- b]thiazole-7-carboxamide |
| 189 | 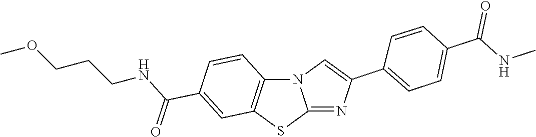 | N-(3-methoxypropyl)-2-(4- (methylcarbamoyl)phenyl) benzo[d]imidazo[2,1- b]thiazole-7-carboxamide |
| 190 | 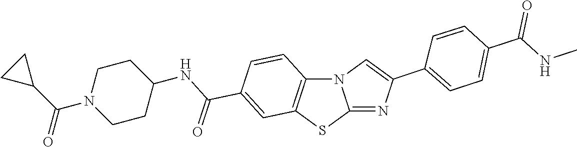 [SMILES: CNC(=O)c1ccc(-c2cn3c(n2)sc2cc(C(=O)NC4CCN(C(=O)C5CC5)CC4)ccc23)cc1] | N-(1- (cyclopropanecarbonyl) piperidin-4-yl)-2-(4- (methylcarbamoyl)phenyl) benzo[d]imidazo[2,1- b]thiazole-7-carboxamide |
| 191 | 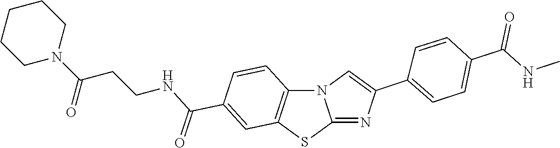 [SMILES: CNC(=O)c1ccc(-c2cn3c(n2)sc2cc(C(=O)NCCC(=O)N4CCCCC4)ccc23)cc1] | 2-(4- (methylcarbamoyl)phenyl)- N-(3-oxo-3-(piperidin-1- yl)propyl)benzo[d]imidazo [2,1-b]thiazole-7-carboxamide |
| 192 | 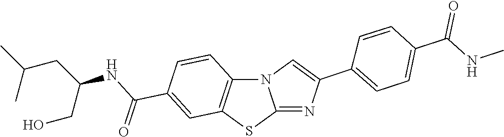 [SMILES: CNC(=O)c1ccc(-c2cn3c(n2)sc2cc(C(=O)N[C@@H](CO)CC(C)C)ccc23)cc1] | (R)-N-(1-hydroxy-4- methylpentan-2-yl)-2-(4- (methylcarbamoyl)phenyl) benzo[d]imidazo[2,1- b]thiazole-7-carboxamide |
| 193 | 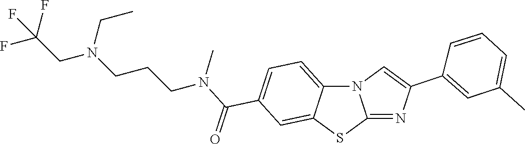 [SMILES: CCN(CCCN(C)C(=O)c1ccc2c(c1)sc1nc(-c3cccc(C)c3)cn12)CC(F)(F)F] | N-(3-(ethyl(2,2,2- trifluoroethyl)amino)propyl) N-methyl-2-(m- tolyl)benzo[d]imidazo[2,1- b]thiazole-7-carboxamide |
| 194 | 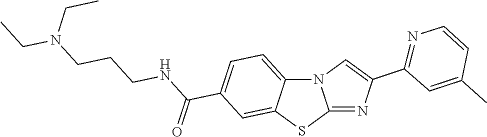 [SMILES: CCN(CC)CCCNC(=O)c1ccc2c(c1)sc1nc(-c3cc(C)ccn3)cn12] | N-(3-(diethylamino)propyl)- 2-(4-methylpyridin-2- yl)benzo[d]imidazo[2,1- b]thiazole-7-carboxamide |
| 195 | 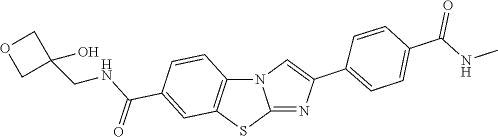 [SMILES: CNC(=O)c1ccc(-c2cn3c(n2)sc2cc(C(=O)NCC4(O)COC4)ccc23)cc1] | N-((3-hydroxyoxetan-3- yl)methyl)-2-(4- (methylcarbamoyl)phenyl) benzo[d]imidazo[2,1- b]thiazole-7-carboxamide |
| 196 | 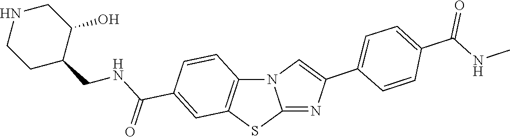 [SMILES: CNC(=O)c1ccc(-c2cn3c(n2)sc2cc(C(=O)NC[C@H]4CCNC[C@@H]4O)ccc23)cc1] | N-(((3R,4R)-3- hydroxypiperidin-4- yl)methyl)-2-(4- (methylcarbamoyl)phenyl) benzo[d]imidazo[2,1- b]thiazole-7-carboxamide |
| 197 | 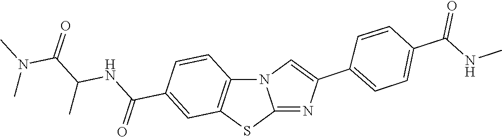 | N-(1-(dimethylamino)-1- oxopropan-2-yl)-2-(4- (methylcarbamoyl)phenyl) benzo[d]imidazo[2,1- b]thiazole-7-carboxamide |
| 198 | 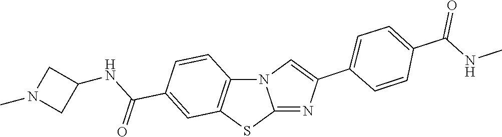 | N-(1-methylazetidin-3-yl)-2- (4- (methylcarbamoyl)phenyl) benzo[d]imidazo[2,1- b]thiazole-7-carboxamide |
| 199 | 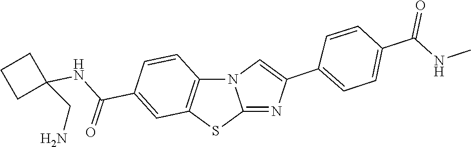 [SMILES: CNC(=O)c1ccc(-c2cn3c(n2)sc2cc(C(=O)NC4(CN)CCC4)ccc23)cc1] | N-(1- (aminomethyl)cyclobutyl)-2- (4- (methylcarbamoyl)phenyl) benzo[d]imidazo[2,1- b]thiazole-7-carboxamide |
| 200 | 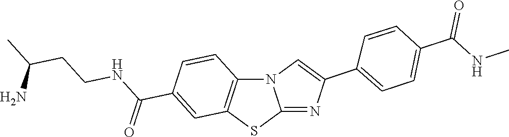 [SMILES: CNC(=O)c1ccc(-c2cn3c(n2)sc2cc(C(=O)NCC[C@H](C)N)ccc23)cc1] | (S)-N-(3-aminobutyl)-2-(4- (methylcarbamoyl)phenyl) benzo[d]imidazo[2,1- b]thiazole-7-carboxamide |
| 201 | 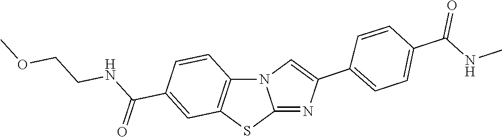 | N-(2-methoxyethyl)-2-(4- (methylcarbamoyl)phenyl) benzo[d]imidazo[2,1- b]thiazole-7-carboxamide |
| 202 | 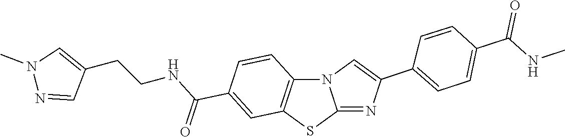 | N-(2-(1-methyl-1H-pyrazol- 4-yl)ethyl)-2-(4- (methylcarbamoyl)phenyl) benzo[d]imidazo[2,1- b]thiazole-7-carboxamide |
| 203 | 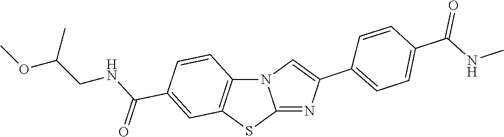 | N-(2-methoxypropyl)-2-(4- (methylcarbamoyl)phenyl) benzo[d]imidazo[2,1- b]thiazole-7-carboxamide |
| 204 | 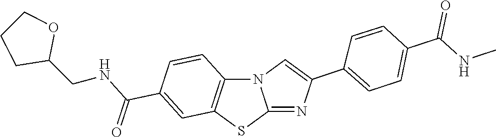 [SMILES: CNC(=O)c1ccc(-c2cn3c(n2)sc2cc(C(=O)NCC4CCCO4)ccc23)cc1] | 2-(4- (methylcarbamoyl)phenyl)- N-((tetrahydrofuran-2- yl)methyl)benzo[d]imidazo [2,1-b]thiazole-7-carboxamide |
| 205 | 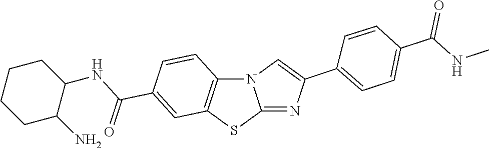 [SMILES: CNC(=O)c1ccc(-c2cn3c(n2)sc2cc(C(=O)NC4CCCCC4N)ccc23)cc1] | N-(2-aminocyclohexyl)-2-(4- (methylcarbamoyl)phenyl) benzo[d]imidazo[2,1- b]thiazole-7-carboxamide |
| 206 | 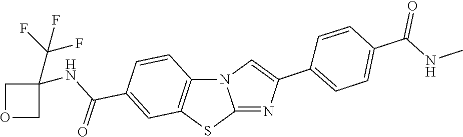 [SMILES: CNC(=O)c1ccc(-c2cn3c(n2)sc2cc(C(=O)NC4(C(F)(F)F)COC4)ccc23)cc1] | 2-(4- (methylcarbamoyl)phenyl)- N-(3- (trifluoromethyl)oxetan-3- yl)benzo[d]imidazo[2,1- b]thiazole-7-carboxamide |
| 207 | 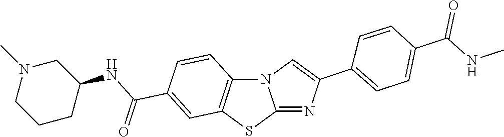 | (S)-2-(4- (methylcarbamoyl)phenyl)- N-(1-methylpiperidin-3- yl)benzo[d]imidazo[2,1- b]thiazole-7-carboxamide |
| 208 | 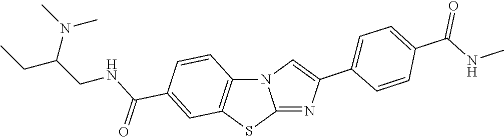 [SMILES: CCC(CNC(=O)c1ccc2c(c1)sc1nc(-c3ccc(C(=O)NC)cc3)cn12)N(C)C] | N-(2-(dimethylamino)butyl)- 2-(4- (methylcarbamoyl)phenyl) benzo[d]imidazo[2,1- b]thiazole-7-carboxamide |
| 209 | 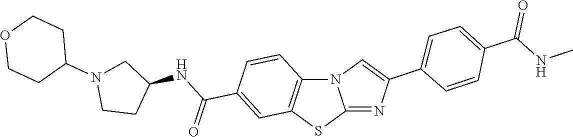 [SMILES: CNC(=O)c1ccc(-c2cn3c(n2)sc2cc(C(=O)N[C@H]4CCN(C5CCOCC5)C4)ccc23)cc1] | (S)-2-(4- (methylcarbamoyl)phenyl)- N-(1-(tetrahydro-2H-pyran- 4-yl)pyrrolidin-3- yl)benzo[d]imidazo[2,1- b]thiazole-7-carboxamide |
| 210 | 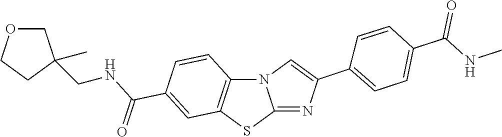 [SMILES: CNC(=O)c1ccc(-c2cn3c(n2)sc2cc(C(=O)NCC4(C)CCOC4)ccc23)cc1] | 2-(4- (methylcarbamoyl)phenyl)- N-((3- methyltetrahydrofuran-3- yl)methyl)benzo[d]imidazo [2,1-b]thiazole-7-carboxamide |
| 211 | 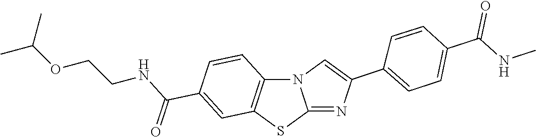 [SMILES: CNC(=O)c1ccc(-c2cn3c(n2)sc2cc(C(=O)NCCOC(C)C)ccc23)cc1] | N-(2-isopropalyethyl)-2-(4- (methylcarbamoyl)phenyl) benzo[d]imidazo[2,1- b]thiazole-7-carboxamide |
| 212 | 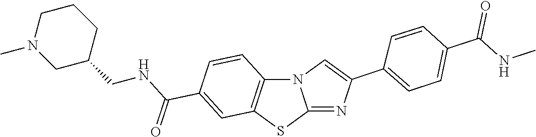 | (R)-2-(4- (methylcarbamoyl)phenyl)- N-((1-methylpiperidin-3- yl)methyl)benzo[d]imidazo [2,1-b]thiazole-7-carboxamide |
| 213 | 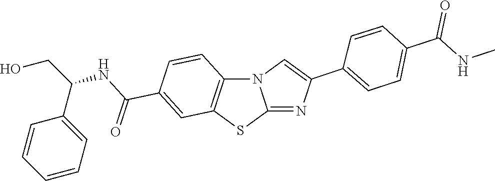 [SMILES: CNC(=O)c1ccc(-c2cn3c(n2)sc2cc(C(=O)N[C@@H](CO)c4ccccc4)ccc23)cc1] | (R)-N-(2-hydroxy-1- phenylethyl)-2-(4- (methylcarbamoyl)phenyl) benzo[d]imidazo[2,1- b]thiazole-7-carboxamide |
| 214 | 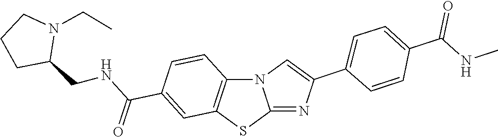 [SMILES: CCN1CCC[C@@H]1CNC(=O)c1ccc2c(c1)sc1nc(-c3ccc(C(=O)NC)cc3)cn12] | (R)-N-((1-ethylpyrrolidin-2- yl)methyl)-2-(4- (methylcarbamoyl)phenyl) benzo[d]imidazo[2,1- b]thiazole-7-carboxamide |
| 215 | 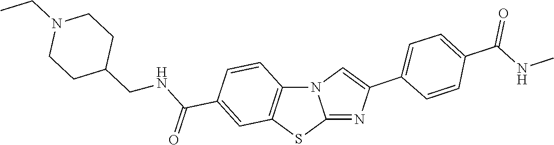 [SMILES: CCN1CCC(CNC(=O)c2ccc3c(c2)sc2nc(-c4ccc(C(=O)NC)cc4)cn23)CC1] | N-((1-ethylpiperidin-4- yl)methyl)-2-(4- (methylcarbamoyl)phenyl) benzo[d]imidazo[2,1- b]thiazole-7-carboxamide |
| 216 | 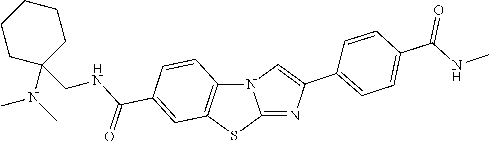 | N-((1- (dimethylamino)cyclohexyl) methyl)-2-(4- (methylcarbamoyl)phenyl) benzo[d]imidazo[2,1- b]thiazole-7-carboxamide |
| 217 | 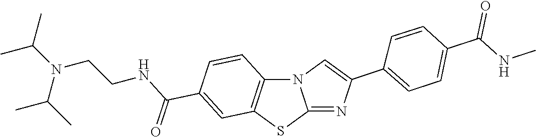 [SMILES: CNC(=O)c1ccc(-c2cn3c(n2)sc2cc(C(=O)NCCN(C(C)C)C(C)C)ccc23)cc1] | N-(2- (diisopropylamino)ethyl)-2- (4- (methylcarbamoyl)phenyl) benzo[d]imidazo[2,1- b]thiazole-7-carboxamide |
| 218 | 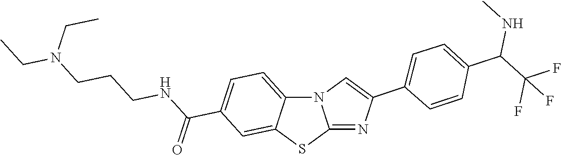 [SMILES: CCN(CC)CCCNC(=O)c1ccc2c(c1)sc1nc(-c3ccc(C(NC)C(F)(F)F)cc3)cn12] | N-(3-(diethylamino)propyl)- 2-(4-(2,2,2-trifluoro-1- (methylamino)ethyl)phenyl) benzo[d]imidazo[2,1- b]thiazole-7-carboxamide |
| 219 | 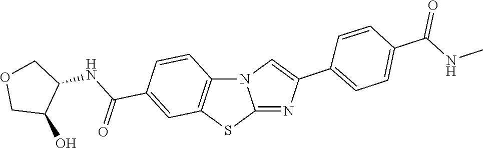 [SMILES: CNC(=O)c1ccc(-c2cn3c(n2)sc2cc(C(=O)N[C@H]4COC[C@@H]4O)ccc23)cc1] | N-((3S,4R)-4- hydroxytetrahydrofuran-3- yl)-2-(4- (methylcarbamoyl)phenyl) benzo[d]imidazo[2,1- b]thiazole-7-carboxamide |
| 220 | 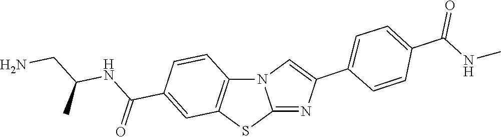 [SMILES: CNC(=O)c1ccc(-c2cn3c(n2)sc2cc(C(=O)N[C@@H](C)CN)ccc23)cc1] | (S)-N-(1-aminopropan-2-yl)- 2-(4- (methylcarbamoyl)phenyl) benzo[d]imidazo[2,1- b]thiazole-7-carboxamide |
| 221 | 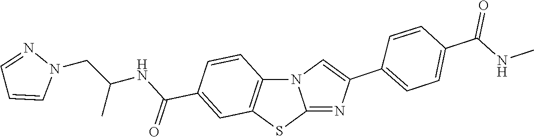 [SMILES: CNC(=O)c1ccc(-c2cn3c(n2)sc2cc(C(=O)NC(C)Cn4cccn4)ccc23)cc1] | N-(1-(1H-pyrazol-1- yl)propan-2-yl)-2-(4- (methylcarbamoyl)phenyl) benzo[d]imidazo[2,1- b]thiazole-7-carboxamide |
| 222 | 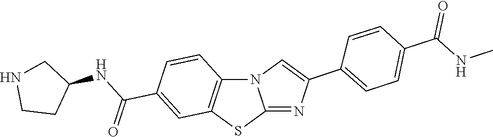 [SMILES: CNC(=O)c1ccc(-c2cn3c(n2)sc2cc(C(=O)N[C@H]4CCNC4)ccc23)cc1] | (S)-2-(4- (methylcarbamoyl)phenyl)- N-(pyrrolidin-3- yl)benzo[d]imidazo[2,1- b]thiazole-7-carboxamide |
| 223 | 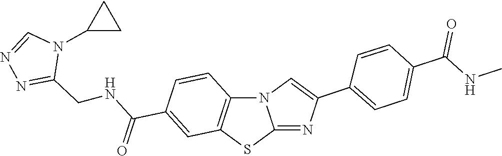 [SMILES: CNC(=O)c1ccc(-c2cn3c(n2)sc2cc(C(=O)NCc4nncn4C4CC4)ccc23)cc1] | N-((4-cyclopropyl-4H-1,2,4- triazol-3-yl)methyl)-2-(4- (methylcarbamoyl)phenyl) benzo[d]imidazo[2,1- b]thiazole-7-carboxamide |
| 224 | 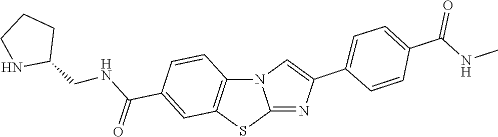 [SMILES: CNC(=O)c1ccc(-c2cn3c(n2)sc2cc(C(=O)NC[C@H]4CCCN4)ccc23)cc1] | (R)-2-(4- (methylcarbamoyl)phenyl)- N-(pyrrolidin-2- ylmethyl)benzo[d]imidazo[2, 1-b]thiazole-7-carboxamide |
| 225 | 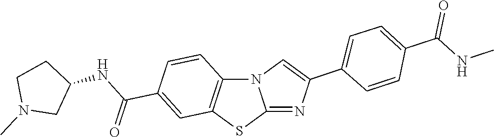 | (S)-2-(4- (methylcarbamoyl)phenyl)- N-(1-methylpyrrolidin-3- yl)benzo[d]imidazo[2,1- b]thiazole-7-carboxamide |
| 226 | 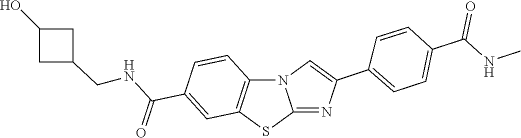 [SMILES: CNC(=O)c1ccc(-c2cn3c(n2)sc2cc(C(=O)NCC4CC(O)C4)ccc23)cc1] | N-((3- hydroxycyclobutyl)methyl)-2- (4- (methylcarbamoyl)phenyl) benzo[d]imidazo[2,1- b]thiazole-7-carboxamide |
| 227 | 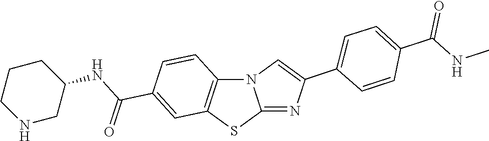 [SMILES: CNC(=O)c1ccc(-c2cn3c(n2)sc2cc(C(=O)N[C@H]4CCCNC4)ccc23)cc1] | (S)-2-(4- (methylcarbamoyl)phenyl)- N-(piperidin-3-yl)benzo[d] imidazo[2,1-b]thiazole-7- carboxamide |
| 228 | 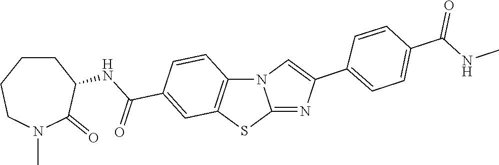 [SMILES: CNC(=O)c1ccc(-c2cn3c(n2)sc2cc(C(=O)N[C@H]4CCCCN(C)C4=O)ccc23)cc1] | (S)-N-(1-methyl-2- oxoazepan-3-yl)-2-(4- (methylcarbamoyl)phenyl) benzo[d]imidazo[2,1- b]thiazole-7-carboxamide |
| 229 | 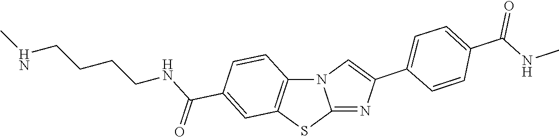 | N-(4-(methylamino)butyl)-2- (4- (methylcarbamoyl)phenyl) benzo[d]imidazo[2,1- b]thiazole-7-carboxamide |
| 230 | 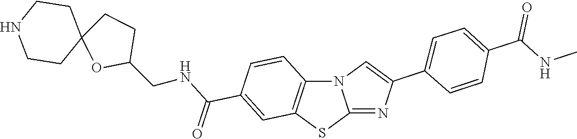 [SMILES: CNC(=O)c1ccc(-c2cn3c(n2)sc2cc(C(=O)NCC4CCC5(CCNCC5)O4)ccc23)cc1] | N-((1-oxa-8- azaspiro[4.5]decan-2- yl)methyl)-2-(4- (methylcarbamoyl)phenyl) benzo[d]imidazo[2,1- b]thiazole-7-carboxamide |
| 231 | 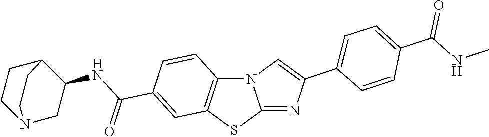 [SMILES: CNC(=O)c1ccc(-c2cn3c(n2)sc2cc(C(=O)N[C@H]4CN5CCC4CC5)ccc23)cc1] | (R)-2-(4- (methylcarbamoyl)phenyl)- N-(quinuelidin-3- yl)benzo[d]imidazo[2,1- b]thiazole-7-carboxamide |
| 232 | 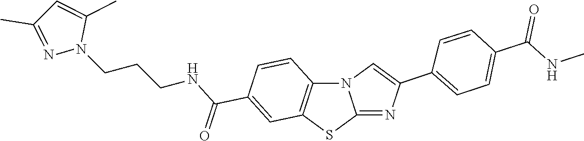 | N-(3-(3,5-dimethyl-1H- pyrazol-1-yl)propyl)-2-(4- (methylcarbamoyl)phenyl) benzo[d]imidazo[2,1- b]thiazole-7-carboxamide |
| 233 | 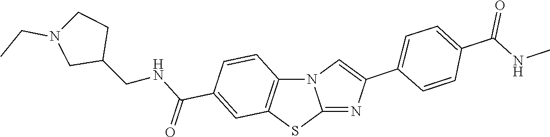 [SMILES: CCN1CCC(CNC(=O)c2ccc3c(c2)sc2nc(-c4ccc(C(=O)NC)cc4)cn23)C1] | N-((1-ethylpyrrolidin-3- yl)methyl)-2-(4- (methylcarbamoyl)phenyl) benzo[d]imidazo[2,1- b]thiazole-7-carboxamide |
| 234 | 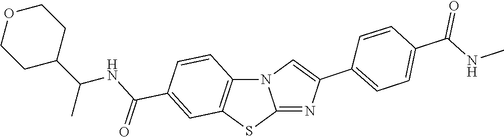 [SMILES: CNC(=O)c1ccc(-c2cn3c(n2)sc2cc(C(=O)NC(C)C4CCOCC4)ccc23)cc1] | 2-(4-(methylcarbamoyl)phenyl)- N-(1-(tetrahydro-2H-pyran- 4-yl)ethyl)benzo[d]imidazo [2,1-b]thiazole-7-carboxamide |
| 235 | 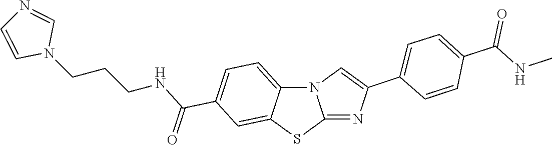 [SMILES: CNC(=O)c1ccc(-c2cn3c(n2)sc2cc(C(=O)NCCCn4ccnc4)ccc23)cc1] | N-(3-(1H-imidazol-1- yl)propyl)-2-(4- (methylcarbamoyl)phenyl) benzo[d]imidazo[2,1- b]thiazole-7-carboxamide |
| 236 | 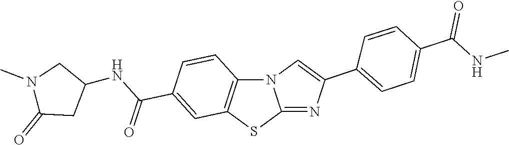 | N-(1-methyl-5-oxopyrrolidin- 3-yl)-2-(4- (methylcarbamoyl)phenyl) benzo[d]imidazo[2,1- b]thiazole-7-carboxamide |
| 237 | 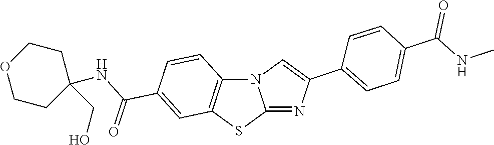 [SMILES: CNC(=O)c1ccc(-c2cn3c(n2)sc2cc(C(=O)NC4(CO)CCOCC4)ccc23)cc1] | N-(4- (hydroxymethyl)tetrahydro- 2H-pyran-4-yl)-2-(4- (methylcarbamoyl)phenyl) benzo[d]imidazo[2,1- b]thiazole-7-carboxamide |
| 238 | 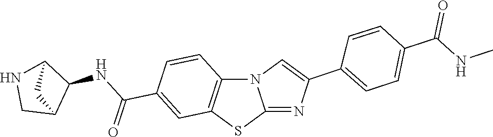 [SMILES: CNC(=O)c1ccc(-c2cn3c(n2)sc2cc(C(=O)N[C@H]4[C@H]5CN[C@@H]4C5)ccc23)cc1] | N-((1R,4R,5S)-2- azabicyclo[2.1.1]hexan-5- (methylcarbamoyl)phenyl) benzo[d]imidazo[2,1- b]thiazole-7-carboxamide |
| 239 | 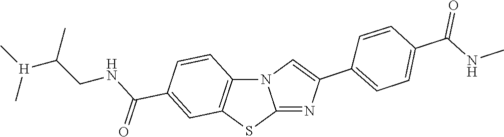 | N-(2- (dimethylamino)propyl)-2- (4- (methylcarbamoyl)phenyl) benzo[d]imidazo[2,1-b] thiazole-7-carboxamide |
| 240 | 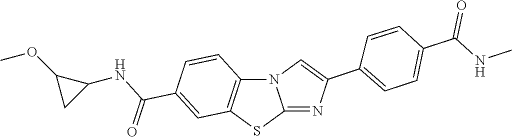 | N-(2-methoxycyclopropyl)-2- (4-(methylcarbamoyl)phenyl) benzo[d]imidazo[2,1-b] thiazole-7-carboxamide |
| 241 | 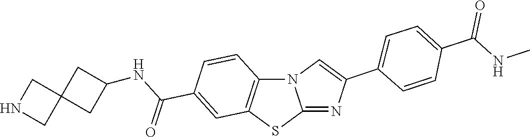 [SMILES: CNC(=O)c1ccc(-c2cn3c(n2)sc2cc(C(=O)NC4CC5(CNC5)C4)ccc23)cc1] | 2-(4- (methylcarbamoyl)phenyl)- N-(2-azaspiro[3.3]heptan-6- yl)benzo[d]imidazo[2,1-b] thiazole-7-carboxamide |
| 242 | 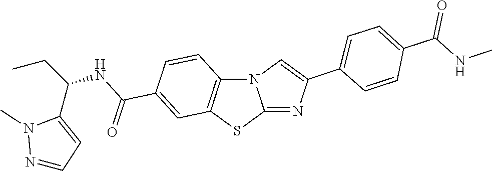 | (S)-N-(1-(1-methyl-1H- pyrazol-5-yl)propyl)-2-(4- (methylcarbamoyl)phenyl) benzo[d]imidazo[2,1-b] thiazole-7-carboxamide |
| 243 | 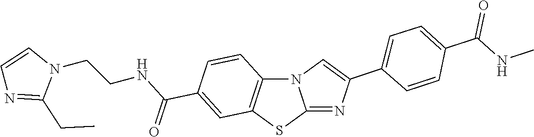 [SMILES: CCc1nccn1CCNC(=O)c1ccc2c(c1)sc1nc(-c3ccc(C(=O)NC)cc3)cn12] | N-(2-(2-ethyl-1H-imidazol-1- yl)ethyl)-2-(4- (methylcarbamoyl)phenyl) benzo[d]imidazo[2,1-b] thiazole-7-carboxamide |
| 244 | 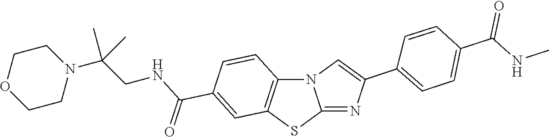 [SMILES: CNC(=O)c1ccc(-c2cn3c(n2)sc2cc(C(=O)NCC(C)(C)N4CCOCC4)ccc23)cc1] | N-(2-methyl-2- morpholinopropyl)-2-(4- (methylcarbamoyl)phenyl) benzo[d]imidazo[2,1-b] thiazole-7-carboxamide |
| 245 | 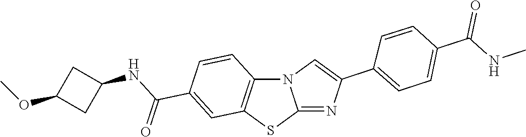 | N-((1s,3s)-3- methoxycyclobutyl)-2-(4- (methylcarbamoyl)phenyl) benzo[d]imidazo[2,1-b] thiazole-7-carboxamide |
| 246 | 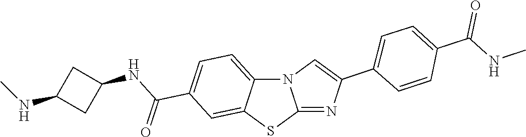 | N-((1s,3s)-3- (methylamino)cyclobutyl)-2- (4- (methylcarbamoyl)phenyl) benzo[d]imidazo[2,1-b] thiazole-7-carboxamide |
| 247 | 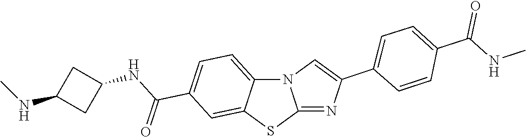 | N-((1r,3r)-3- (methylamino)cyclobutyl)-2- (4- (methylcarbamoyl)phenyl) benzo[d]imidazo[2,1-b] thiazole-7-carboxamide |
| 248 | 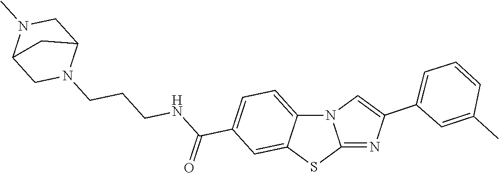 | N-(3-(5-methyl-2,5- diazabicyclo[2.2.1]heptan-2- yl)propyl)-2-(m- tolyl)benzo[d]imidazo[2,1- b]thiazole-7-carboxamide |
| 249 | 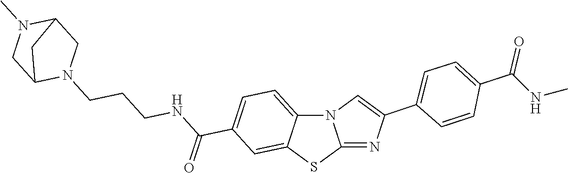 | N-(3-(5-methyl-2,5- diazabicyclo[2.2.1]heptan-2- yl)propyl)-2-(4- (methylcarbamoyl)phenyl) benzo[d]imidazo[2,1- b]thiazole-7-carboxamide |
| 250 | 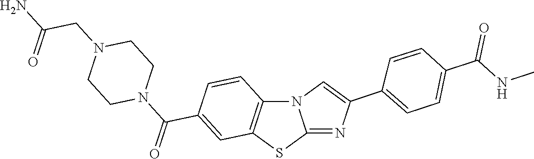 [SMILES: CNC(=O)c1ccc(-c2cn3c(n2)sc2cc(C(=O)N4CCN(CC(N)=O)CC4)ccc23)cc1] | 4-(7-(4-(2-amino-2- oxoethyl)piperazine-1- carbonyl)benzo[d]imidazo [2,1-b]thiazol-2-yl)-N- methylbenzamide |
| 251 | 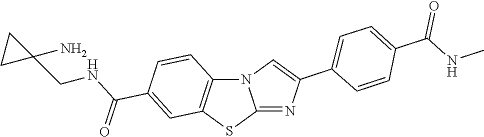 [SMILES: CNC(=O)c1ccc(-c2cn3c(n2)sc2cc(C(=O)NCC4(N)CC4)ccc23)cc1] | N-((1- aminocyclopropyl)methyl)-2- (4- (methylcarbamoyl)phenyl) benzo[d]imidazo[2,1- b]thiazole-7-carboxamide |
| 252 | 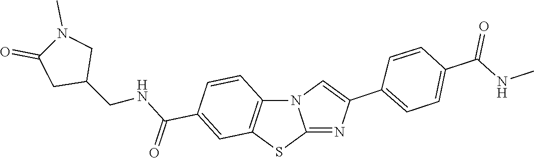 [SMILES: CNC(=O)c1ccc(-c2cn3c(n2)sc2cc(C(=O)NCC4CC(=O)N(C)C4)ccc23)cc1] | N-((1-methyl-5- oxopyrrolidin-3-yl)methyl)- 2-(4- (methylcarbamoyl)phenyl) benzo[d]imidazo[2,1- b]thiazole-7-carboxamide |
| 253 | 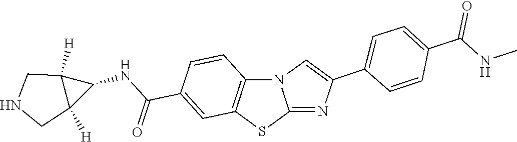 [SMILES: CNC(=O)c1ccc(-c2cn3c(n2)sc2cc(C(=O)N[C@H]4[C@@H]5CNC[C@@H]54)ccc23)cc1] | N-((1R,5S,6s)-3- azabicyclo[3.1.0]hexan-6- yl)-2-(4- (methylcarbamoyl)phenyl) benzo[d]imidazo[2,1- b]thiazole-7-carboxamide |
| 254 | 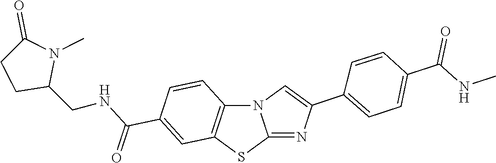 [SMILES: CNC(=O)c1ccc(-c2cn3c(n2)sc2cc(C(=O)NCC4CCC(=O)N4C)ccc23)cc1] | N-((1-methyl-5- oxopyrrolidin-2-yl)methyl) 2-(4- (methylcarbamoyl)phenyl) benzo[d]imidazo[2,1- b]thiazole-7-carboxamide |
| 255 | 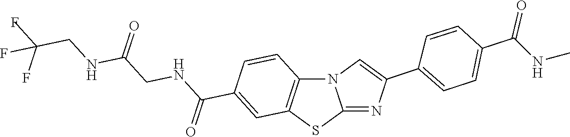 | 2-(4- (methylcarbamoyl)phenyl)- N-(2-oxo-2-((2,2,2- trifluoroethyl)amino)ethyl) benzo[d]imidazo[2,1- b]thiazole-7-carboxamide |
| 256 | 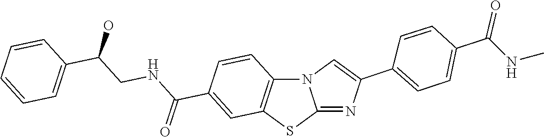 [SMILES: CNC(=O)c1ccc(-c2cn3c(n2)sc2cc(C(=O)NC[C@H](O)c4ccccc4)ccc23)cc1] | (R)-N-(2-hydroxy-2- phenylethyl)-2-(4- (methylcarbamoyl)phenyl) benzo[d]imidazo[2,1- b]thiazole-7-carboxamide |
| 257 | 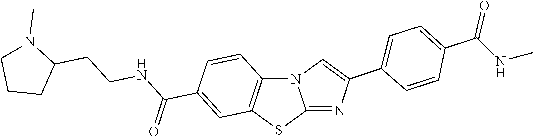 [SMILES: CNC(=O)c1ccc(-c2cn3c(n2)sc2cc(C(=O)NCCC4CCCN4C)ccc23)cc1] | 2-(4- (methylcarbamoyl)phenyl)- N-(2-(1-methylpyrrolidin-2- yl)ethyl)benzo[d]imidazo [2,1-b]thiazole-7-carboxamide |
| 258 | 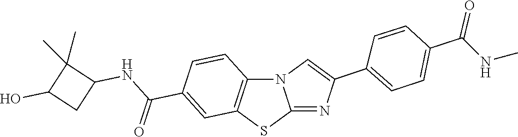 [SMILES: CNC(=O)c1ccc(-c2cn3c(n2)sc2cc(C(=O)NC4CC(O)C4(C)C)ccc23)cc1] | N-(3-hydroxy-2,2- dimethylcyclobutyl)-2-(4- (methylcarbamoyl)phenyl) benzo[d]imidazo[2,1- b]thiazole-7-carboxamide |
| 259 | 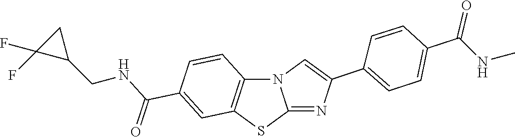 [SMILES: CNC(=O)c1ccc(-c2cn3c(n2)sc2cc(C(=O)NCC4CC4(F)F)ccc23)cc1] | N-((2,2- difluorocyclopropyl)methyl)- 2-(4- (methylcarbamoyl)phenyl) benzo[d]imidazo[2,1- b]thiazole-7-carboxamide |
| 260 | 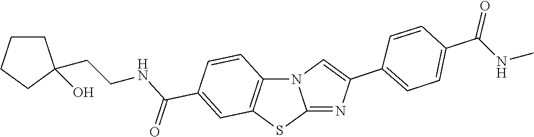 [SMILES: CNC(=O)c1ccc(-c2cn3c(n2)sc2cc(C(=O)NCCC4(O)CCCC4)ccc23)cc1] | N-(2-(1- hydroxycyclopentyl)ethyl)-2- (4-(methylcarbamoyl)phenyl) benzo[d]imidazo[2,1- b]thiazole-7-carboxamide |
| 261 | 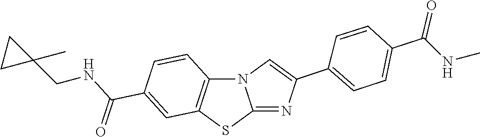 [SMILES: CNC(=O)c1ccc(-c2cn3c(n2)sc2cc(C(=O)NCC4(C)CC4)ccc23)cc1] | 2-(4- (methylcarbamoyl)phenyl) N-((1- methylcyclopropyl)methyl) benzo[d]imidazo[2,1- b]thiazole-7-carboxamide |
| 262 | 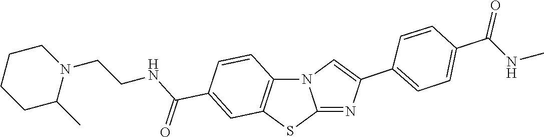 [SMILES: CNC(=O)c1ccc(-c2cn3c(n2)sc2cc(C(=O)NCCN4CCCCC4C)ccc23)cc1] | 2-(4- (methylcarbamoyl)phenyl) N-(2-(2-methylpiperidin-1- yl)ethyl)benzo[d]imidazo [2,1-b]thiazole-7-carboxamide |
| 263 | 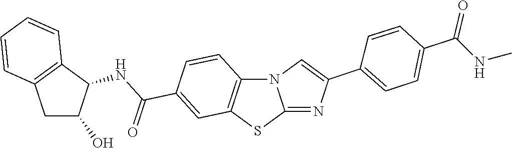 [SMILES: CNC(=O)c1ccc(-c2cn3c(n2)sc2cc(C(=O)N[C@H]4c5ccccc5C[C@H]4O)ccc23)cc1] | N-((1S,2R)-2-hydroxy-2,3- dihydro-1H-inden-1-yl)-2-(4- (methylcarbamoyl)phenyl) benzo[d]imidazo[2,1- b]thiazole-7-carboxamide |
| 264 | 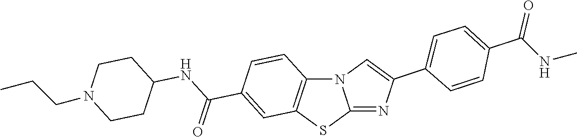 [SMILES: CCCN1CCC(NC(=O)c2ccc3c(c2)sc2nc(-c4ccc(C(=O)NC)cc4)cn23)CC1] | 2-(4- (methylcarbamoyl)phenyl)- N-(1-propylpiperidin-4- yl)benzo[d]imidazo[2,1- b]thiazole-7-carboxamide |
| 265 | 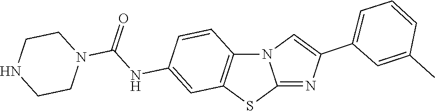 [SMILES: Cc1cccc(-c2cn3c(n2)sc2cc(NC(=O)N4CCNCC4)ccc23)c1] | N-(2-(m- tolyl)benzo[d]imidazo[2,1- b]thiazol-7-yl)piperazine-1- carboxamide |
| 266 | 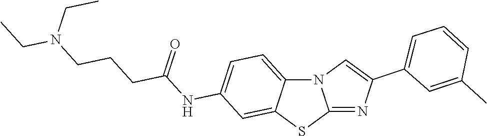 [SMILES: CCN(CC)CCCC(=O)Nc1ccc2c(c1)sc1nc(-c3cccc(C)c3)cn12] | 4-(diethylamino)-N-(2-(m-tolyl) benzo[d]imidazo[2,1- b]thiazol-7-yl)butanamide |
| 267 | 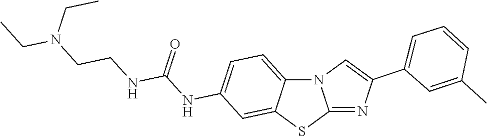 [SMILES: CCN(CC)CCNC(=O)Nc1ccc2c(c1)sc1nc(-c3cccc(C)c3)cn12] | 1-(2-(diethylamino)ethyl)-3- (2-(m- tolyl)benzo[d]imidazo[2,1- b]thiazol-7-yl)urea |
| 268 | 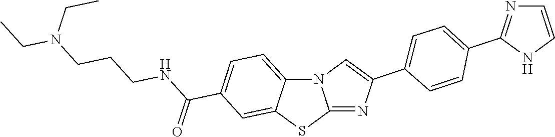 [SMILES: CCN(CC)CCCNC(=O)c1ccc2c(c1)sc1nc(-c3ccc(-c4ncc[nH]4)cc3)cn12] | 2-(4-(1H-imidazol-2- yl)phenyl)-N-(3- (diethylamino)propyl) benzo[d]imidazo[2,1-b] thiazole-7-carboxamide |
| 269 | 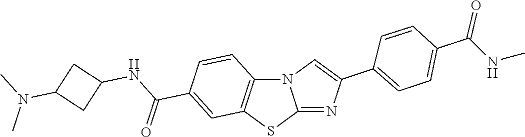 | N-(3- (dimethylamino)cyclobutyl)- 2-(4- (methylcarbamoyl)phenyl) benzo[d]imidazo[2,1- b]thiazole-7-carboxamide |
| 270 | 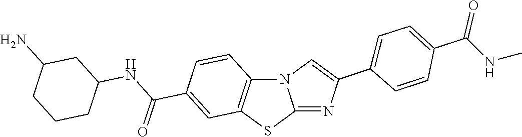 [SMILES: CNC(=O)c1ccc(-c2cn3c(n2)sc2cc(C(=O)NC4CCCC(N)C4)ccc23)cc1] | N-(3-aminocyclohexyl)-2-(4- (methylcarbamoyl)phenyl) benzo[d]imidazo[2,1- b]thiazole-7-carboxamide |
| 271 | 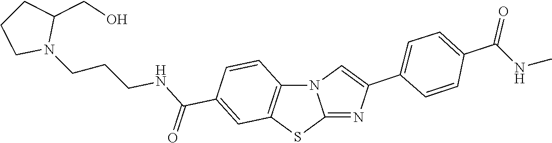 [SMILES: CNC(=O)c1ccc(-c2cn3c(n2)sc2cc(C(=O)NCCCN4CCCC4CO)ccc23)cc1] | N-(3-(2- (hydroxymethyl)pyrrolidin-1- yl)propyl)-2-(4- (methylcarbamoyl)phenyl) benzo[d]imidazo[2,1- b]thiazole-7-carboxamide |
| 272 | 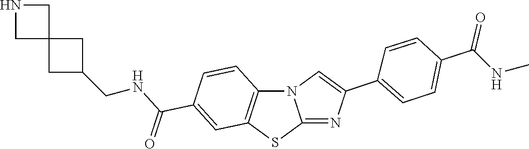 [SMILES: CNC(=O)c1ccc(-c2cn3c(n2)sc2cc(C(=O)NCC4CC5(CNC5)C4)ccc23)cc1] | N-((2-azaspiro[3.3]heptan- 6-yl)methyl)-2-(4- (methylcarbamoyl)phenyl) benzo[d]imidazo[2,1- b]thiazole-7-carboxamide |
| 273 | 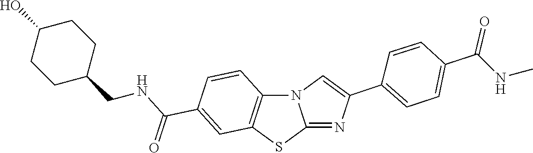 [SMILES: CNC(=O)c1ccc(-c2cn3c(n2)sc2cc(C(=O)NC[C@H]4CC[C@H](O)CC4)ccc23)cc1] | N-(((1r,4r)-4- hydroxycyclohexyl)methyl)- 2-(4- (methylcarbamoyl)phenyl) benzo[d]imidazo[2,1- b]thiazole-7-carboxamide |
| 274 | 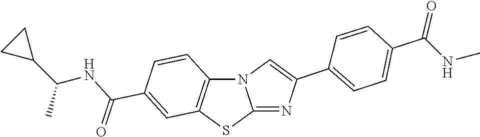 [SMILES: CNC(=O)c1ccc(-c2cn3c(n2)sc2cc(C(=O)N[C@H](C)C4CC4)ccc23)cc1] | (R)-N-(1-cyclopropylethyl)- 2-(4- (methylcarbamoyl)phenyl) benzo[d]imidazo[2,1- b]thiazole-7-carboxamide |
| 275 | 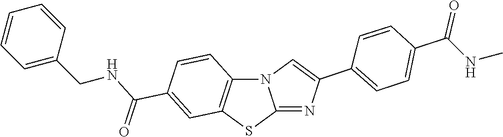 [SMILES: CNC(=O)c1ccc(-c2cn3c(n2)sc2cc(C(=O)NCc4ccccc4)ccc23)cc1] | N-benzyl-2-(4- (methylcarbamoyl)phenyl) benzo[d]imidazo[2,1- b]thiazole-7-carboxamide |
| 276 | 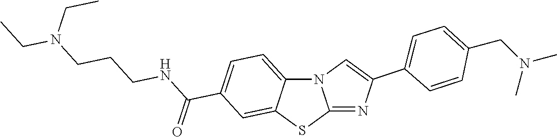 [SMILES: CCN(CC)CCCNC(=O)c1ccc2c(c1)sc1nc(-c3ccc(CN(C)C)cc3)cn12] | N-(3-(diethylamino)propyl)- 2-(4- ((dimethylamino)methyl) phenyl)benzo[d]imidazo[2,1- b]thiazole-7-carboxamide |
| 277 | 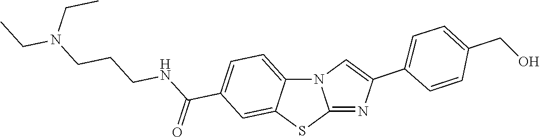 [SMILES: CCN(CC)CCCNC(=O)c1ccc2c(c1)sc1nc(-c3ccc(CO)cc3)cn12] | N-(3-(diethylamino)propyl)- 2-(4-(hydroxymethyl)phenyl) benzo[d]imidazo[2,1-b] thiazole-7-carboxamide |
| 278 | 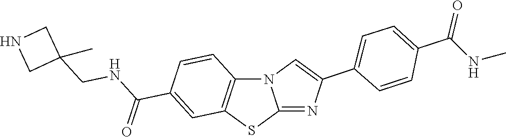 [SMILES: CNC(=O)c1ccc(-c2cn3c(n2)sc2cc(C(=O)NCC4(C)CNC4)ccc23)cc1] | N-((3-methylazetidin-3- yl)methyl)-2-(4- (methylcarbamoyl)phenyl) benzo[d]imidazo[2,1- b]thiazole-7-carboxamide |
| 279 | 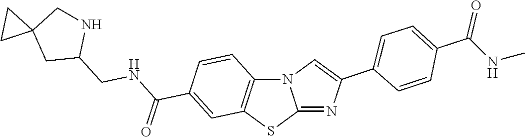 [SMILES: CNC(=O)c1ccc(-c2cn3c(n2)sc2cc(C(=O)NCC4CC5(CC5)CN4)ccc23)cc1] | N-((5-azaspiro[2.4]heptan- 6-yl)methyl)-2-(4- (methylcarbamoyl)phenyl) benzo[d]imidazo[2,1- b]thiazole-7-carboxamide |
| 280 | 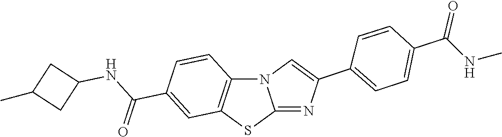 | 2-(4- (methylcarbamoyl)phenyl)- N-(3- methylcyclobutyl)benzo[d] imidazo[2,1-b]thiazole-7- carboxamide |
| 281 | 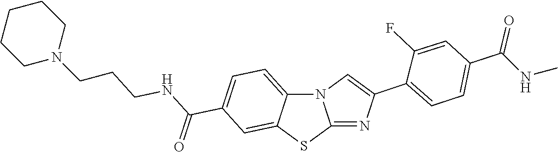 [SMILES: CNC(=O)c1ccc(-c2cn3c(n2)sc2cc(C(=O)NCCCN4CCCCC4)ccc23)c(F)c1] | 2-(2-fluoro-4- (methylcarbamoyl)phenyl)- N-(3-(piperidin-1- yl)propyl)benzo[d]imidazo [2,1-b]thiazole-7- carboxamide |
| 282 | 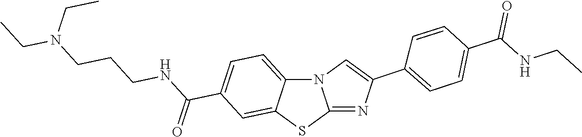 [SMILES: CCNC(=O)c1ccc(-c2cn3c(n2)sc2cc(C(=O)NCCCN(CC)CC)ccc23)cc1] | N-(3-(diethylamino)propyl)- 2-(4- (ethylcarbamoyl)phenyl) benzo[d]imidazo[2,1-b]thiazole- 7-carboxamide |
| 283 | 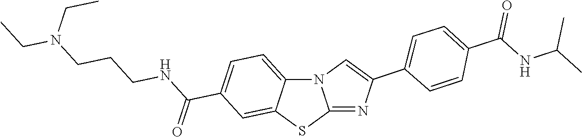 [SMILES: CCN(CC)CCCNC(=O)c1ccc2c(c1)sc1nc(-c3ccc(C(=O)NC(C)C)cc3)cn12] | N-(3-(diethylamino)propyl)- 2-(4- (isopropylcarbamoyl)phenyl) benzo[d]imidazo[2,1- b]thiazole-7-carboxamide |
| 284 | 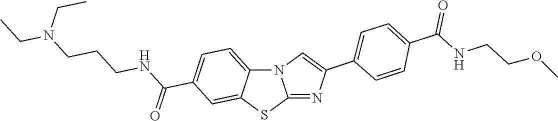 [SMILES: CCN(CC)CCCNC(=O)c1ccc2c(c1)sc1nc(-c3ccc(C(=O)NCCOC)cc3)cn12] | N-(3-(diethylamino)propyl)- 2-(4-((2- methoxyethyl)carbamoyl) phenyl)benzo[d]imidazo[2,1- b]thiazole-7-carboxamide |
| 285 | 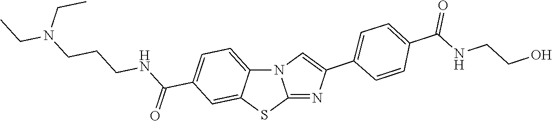 [SMILES: CCN(CC)CCCNC(=O)c1ccc2c(c1)sc1nc(-c3ccc(C(=O)NCCO)cc3)cn12] | N-(3-(diethylamino)propyl)- 2-(4-((2- hydroxyethyl)carbamoyl) phenyl)benzo[d]imidazo[2,1- b]thiazole-7-carboxamide |
| 286 | 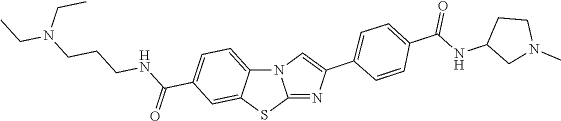 [SMILES: CCN(CC)CCCNC(=O)c1ccc2c(c1)sc1nc(-c3ccc(C(=O)NC4CCN(C)C4)cc3)cn12] | N-(3-(diethylamino)propyl)- 2-(4-((1-methylpyrrolidin-3- yl)carbamoyl)phenyl)benzo [d]imidazo[2,1-b]thiazole-7- carboxamide |
| 287 | 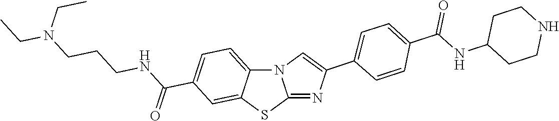 [SMILES: CCN(CC)CCCNC(=O)c1ccc2c(c1)sc1nc(-c3ccc(C(=O)NC4CCNCC4)cc3)cn12] | N-(3-(diethylamino)propyl)- 2-(4-(piperidin-4- ylcarbamoyl)phenyl)benzo [d]imidazo[2,1-b]thiazole-7- carboxamide |
| 288 | 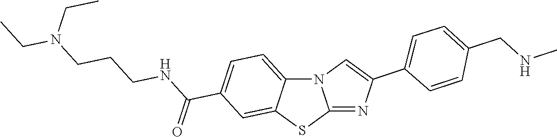 [SMILES: CCN(CC)CCCNC(=O)c1ccc2c(c1)sc1nc(-c3ccc(CNC)cc3)cn12] | N-(3-(diethylamino)propyl)- 2-(4- ((methylamino)methyl)phenyl) benzo[d]imidazo[2,1- b]thiazole-7-carboxamide |
| 289 | 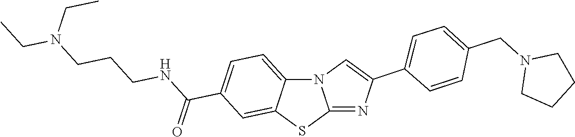 [SMILES: CCN(CC)CCCNC(=O)c1ccc2c(c1)sc1nc(-c3ccc(CN4CCCC4)cc3)cn12] | N-(3-(diethylamino)propyl)- 2-(4-(pyrrolidin-1- ylmethyl)phenyl)benzo[d] imidazo[2,1-b]thiazole-7- carboxamide |
| 290 | 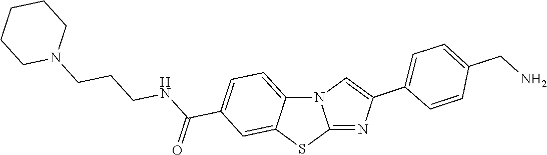 [SMILES: NCc1ccc(-c2cn3c(n2)sc2cc(C(=O)NCCCN4CCCCC4)ccc23)cc1] | 2-(4-(aminomethyl)phenyl)- N-(3-(piperidin-1- yl)propyl)benzo[d]imidazo [2,1-b]thiazole-7-carboxamide |
| 291 | 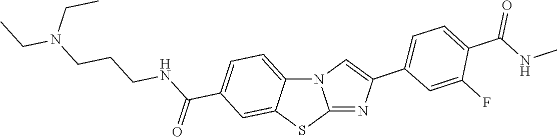 [SMILES: CCN(CC)CCCNC(=O)c1ccc2c(c1)sc1nc(-c3ccc(C(=O)NC)c(F)c3)cn12] | N-(3-(diethylamino)propyl)- 2-(3-fluoro-4- (methylcarbamoyl)phenyl) benzo[dlimidazo[2,1-b] thiazole-7-carboxamide |
| 292 | 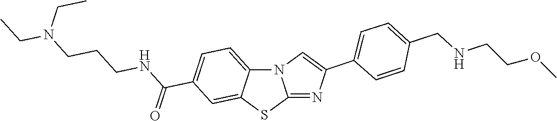 [SMILES: CCN(CC)CCCNC(=O)c1ccc2c(c1)sc1nc(-c3ccc(CNCCOC)cc3)cn12] | N-(3-(diethylamino)propyl)- 2-(4-(((2- methoxyethyl)amino)methyl) phenyl)benzo[d]imidazo[2,1- b]thiazole-7-carboxamide |
| 293 | 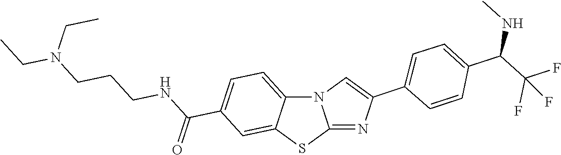 [SMILES: CCN(CC)CCCNC(=O)c1ccc2c(c1)sc1nc(-c3ccc([C@@H](NC)C(F)(F)F)cc3)cn12] | (R)-N-(3- (diethylamino)propyl)-2-(4- (2,2,2-trifluoro-1- (methylamino)ethyl)phenyl) benzo[d]imidazo[2,1- b]thiazole-7-carboxamide |
| 294 | 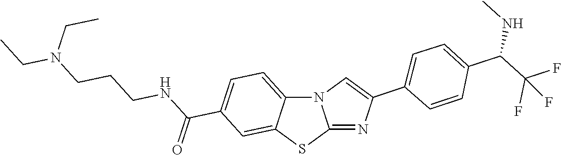 [SMILES: CCN(CC)CCCNC(=O)c1ccc2c(c1)sc1nc(-c3ccc([C@H](NC)C(F)(F)F)cc3)cn12] | (S)-N-(3- (diethylamino)propyl)-2-(4- (2,2,2-trifluoro-1- (methylamino)ethyl)phenyl) benzo[d]imidazo[2,1- b]thiazole-7-carboxamide |
| 295 | 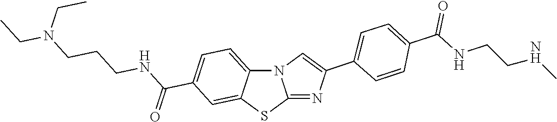 [SMILES: CCN(CC)CCCNC(=O)c1ccc2c(c1)sc1nc(-c3ccc(C(=O)NCCNC)cc3)cn12] | N-(3-(diethylamino)propyl)- 2-(4-((2- (methylamino)ethyl) carbamoyl)phenyl)benzo[d] imidazo[2,1-b]thiazole-7- carboxamide |
| 296 | 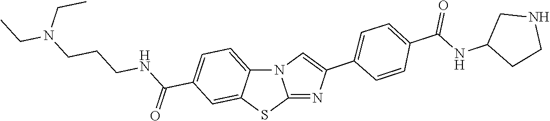 [SMILES: CCN(CC)CCCNC(=O)c1ccc2c(c1)sc1nc(-c3ccc(C(=O)NC4CCNC4)cc3)cn12] | N-(3-(diethylamino)propyl)- 2-(4-(pyrrolidin-3- ylcarbamoyl)phenyl)benzo[d] imidazo[2,1-b]thiazole-7- carboxamide |
| 297 | 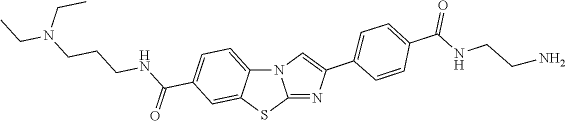 [SMILES: CCN(CC)CCCNC(=O)c1ccc2c(c1)sc1nc(-c3ccc(C(=O)NCCN)cc3)cn12] | 2-(4-((2- aminoethyl)carbamoyl) phenyl)-N-(3- (diethylamino)propyl)benzo [d]imidazo[2,1-b]thiazole-7- carboxamide |
| 298 | 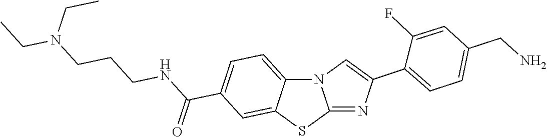 [SMILES: CCN(CC)CCCNC(=O)c1ccc2c(c1)sc1nc(-c3ccc(CN)cc3F)cn12] | 2-(4-(aminomethyl)-2- fluorophenyl)-N-(3- (diethylamino)propyl)benzo [d]imidazo[2,1-b]thiazole-7- carboxamide |
| 299 | 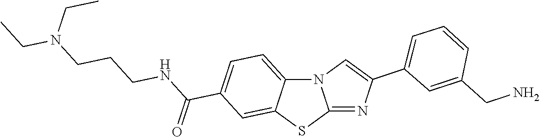 [SMILES: CCN(CC)CCCNC(=O)c1ccc2c(c1)sc1nc(-c3cccc(CN)c3)cn12] | 2-(3-(aminomethyl)phenyl)- N-(3-(diethylamino)propyl) benzo[d]imidazo[2,1-b] thiazole-7-carboxamide |
| 300 | 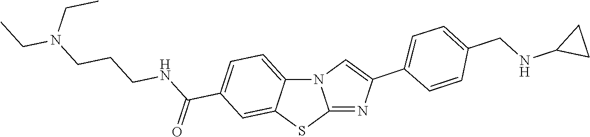 [SMILES: CCN(CC)CCCNC(=O)c1ccc2c(c1)sc1nc(-c3ccc(CNC4CC4)cc3)cn12] | 2-(4- ((cyclopropylamino)methyl) phenyl)-N-(3- (diethylamino)propyl)benzo [d]imidazo[2,1-b]thiazole-7- carboxamide |
| 301 | 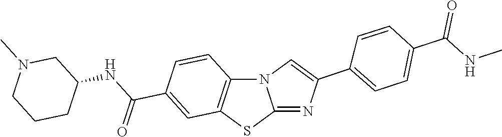 | (R)-2-(4- (methylcarbamoyl)phenyl)- N-(1-methylpiperidin-3- yl)benzo[d]imidazo[2,1- b]thiazole-7-carboxamide |
| 302 | 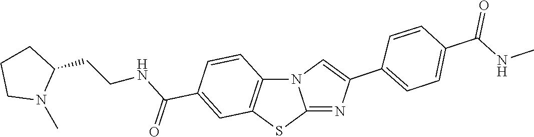 [SMILES: CNC(=O)c1ccc(-c2cn3c(n2)sc2cc(C(=O)NCC[C@H]4CCCN4C)ccc23)cc1] | (R)-2-(4- (methylcarbamoyl)phenyl)- N-(2-(1-methylpyrrolidin-2- yl)ethyl)benzo[d]imidazo [2,1-b]thiazole-7-carboxamide |
| 303 | 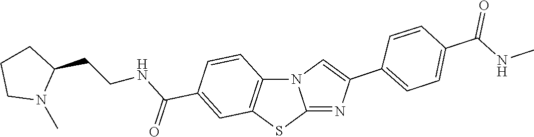 [SMILES: CNC(=O)c1ccc(-c2cn3c(n2)sc2cc(C(=O)NCC[C@@H]4CCCN4C)ccc23)cc1] | (S)-2-(4- (methylcarbamoyl)phenyl)- N-(2-(1-methylpyrrolidin-2- yl)ethyl)benzo[d]imidazo [2,1-b]thiazole-7-carboxamide |
| 304 | 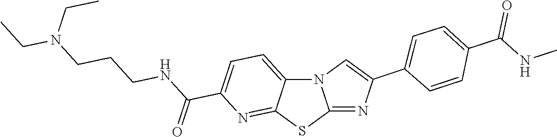 [SMILES: CCN(CC)CCCNC(=O)c1ccc2c(n1)sc1nc(-c3ccc(C(=O)NC)cc3)cn12] | N-(3-(diethylamino)propyl)- 2-(4- (methylcarbamoyl)phenyl) imidazo[2′,1′:2,3]thiazolo[5,4- b]pyridine-7-carboxamide |
| 305 | 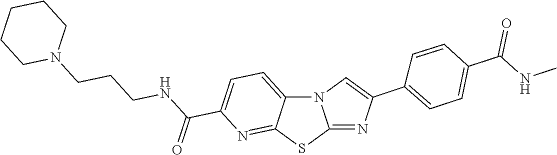 [SMILES: CNC(=O)c1ccc(-c2cn3c(n2)sc2nc(C(=O)NCCCN4CCCCC4)ccc23)cc1] | 2-(4- (methylcarbamoyl)phenyl)- N-(3-(piperidin-1- yl)propyl)imidazo[2′,1′:2,3] thiazolo[5,4-b]pyridine-7- carboxamide |
| 306 | 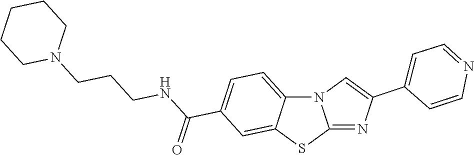 [SMILES: O=C(NCCCN1CCCCC1)c1ccc2c(c1)sc1nc(-c3ccncc3)cn12] | N-(3-(piperidin-1-yl)propyl)- 2-(pyridin-4- yl)benzo[d]imidazo[2,1- b]thiazole-7-carboxamide |
| 307 | 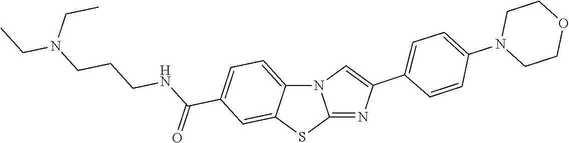 [SMILES: CCN(CC)CCCNC(=O)c1ccc2c(c1)sc1nc(-c3ccc(N4CCOCC4)cc3)cn12] | N-(3-(diethylamino)propyl)- 2-(4- morpholinophenyl)benzo[d] imidazo[2,1-b]thiazole-7- carboxamide |
| 308 | 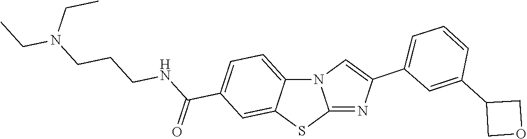 [SMILES: CCN(CC)CCCNC(=O)c1ccc2c(c1)sc1nc(-c3cccc(C4COC4)c3)cn12] | N-(3-(diethylamino)propyl)- 2-(3-(oxetan-3- yl)phenyl)benzo[d]imidazo [2,1-b]thiazole-7-carboxamide |
| 309 | 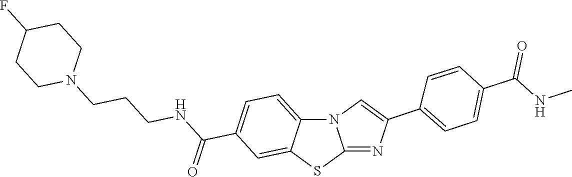 [SMILES: CNC(=O)c1ccc(-c2cn3c(n2)sc2cc(C(=O)NCCCN4CCC(F)CC4)ccc23)cc1] | N-(3-(4-fluoropiperidin-1- yl)propyl)-2-(4- (methylcarbamoyl)phenyl) benzo[d]imidazo[2,1- b]thiazole-7-carboxamide |
| 310 | 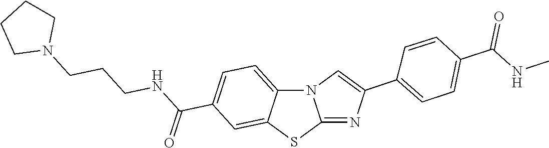 [SMILES: CNC(=O)c1ccc(-c2cn3c(n2)sc2cc(C(=O)NCCCN4CCCC4)ccc23)cc1] | 2-(4- (methylcarbamoyl)phenyl)- N-(3-(pyrrolidin-1- yl)propyl)benzo[d]imidazo [2,1-b]thiazole-7-carboxamide |
| 311 | 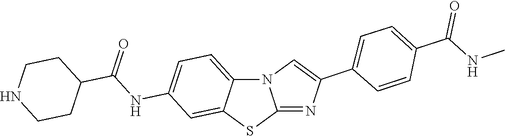 [SMILES: CNC(=O)c1ccc(-c2cn3c(n2)sc2cc(NC(=O)C4CCNCC4)ccc23)cc1] | (N-(2-(4-methylcarbamoyl) phenyl)benzo[d]imidazo [2,1-b]thiazol- 7-yl)piperidine-4- carboxamide |
| 312 | 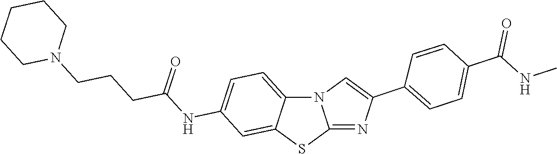 [SMILES: CNC(=O)c1ccc(-c2cn3c(n2)sc2cc(NC(=O)CCCN4CCCCC4)ccc23)cc1] | N-methyl-4-(7-(4-(piperidin- 1-yl)butanamido)benzo[d] imidazo[2,1-b]thiazol-2- yl)benzamide |
| 313 | 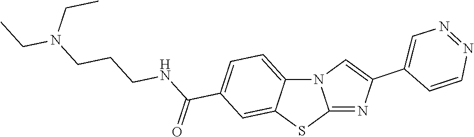 [SMILES: CCN(CC)CCCNC(=O)c1ccc2c(c1)sc1nc(-c3ccnnc3)cn12] | N-(3-(diethylamino)propyl)- 2-(pyridazin-4- yl)benzo[d]imidazo[2,1- b]thiazole-7-carboxamide |
| 314 | 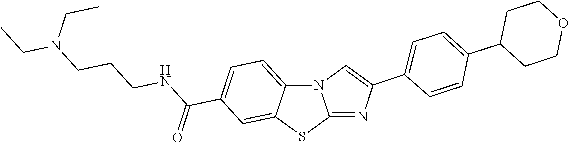 [SMILES: CCN(CC)CCCNC(=O)c1ccc2c(c1)sc1nc(-c3ccc(C4CCOCC4)cc3)cn12] | N-(3-(diethylamino)propyl)- 2-(4-(tetrahydro-2H-pyran- 4- yl)phenyl)benzo[d]imidazo [2,1-b]thiazole-7-carboxamide |
| 315 | 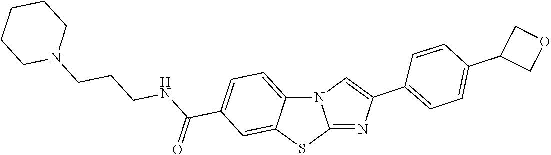 [SMILES: O=C(NCCCN1CCCCC1)c1ccc2c(c1)sc1nc(-c3ccc(C4COC4)cc3)cn12] | 2-(4-(oxetan-3-yl)phenyl)-N- (3-(piperidin-1- yl)propyl)benzo[d]imidazo [2,1-b]thiazole-7-carboxamide |
| 316 | 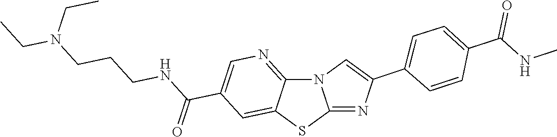 [SMILES: CCN(CC)CCCNC(=O)c1cnc2c(c1)sc1nc(-c3ccc(C(=O)NC)cc3)cn12] | N-(3-(diethylamino)propyl)- 2-(4- (methylcarbamoyl)phenyl) imidazo[2′,1′:2,3]thiazolo[4,5- b]pyridine-7-carboxamide |
| 317 | 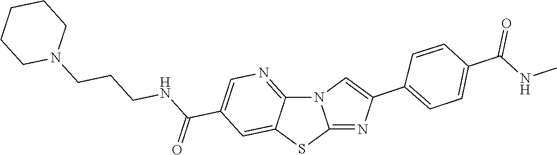 [SMILES: CNC(=O)c1ccc(-c2cn3c(n2)sc2cc(C(=O)NCCCN4CCCCC4)cnc23)cc1] | 2-(4- (methylcarbamoyl)phenyl)- N-(3-(piperidin-1- yl)propyl)imidazo[2′,1′:2,3] thiazolo[4,5-b]pyridine-7- carboxamide |
| 318 | 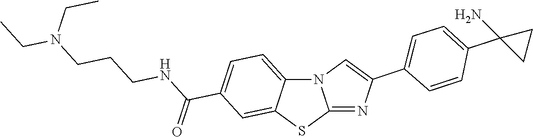 [SMILES: CCN(CC)CCCNC(=O)c1ccc2c(c1)sc1nc(-c3ccc(C4(N)CC4)cc3)cn12] | 2-(4-(1- aminocyclopropyl)phenyl)- N-(3- (diethylamino)propyl)benzo [d]imidazo[2,1-b]thiazole-7- carboxamide |
| 319 | 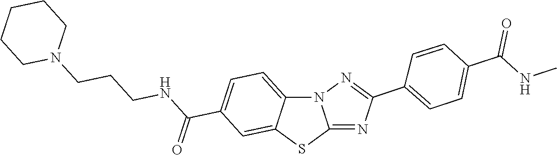 [SMILES: CNC(=O)c1ccc(-c2nc3sc4cc(C(=O)NCCCN5CCCCC5)ccc4n3n2)cc1] | 2-(4- (methylcarbamoyl)phenyl)- N-(3-(piperidin-1- yl)propyl)benzo[4,5]thiazolo [3,2-b][1,2,4]triazole-6- carboxamide |
| 320 | 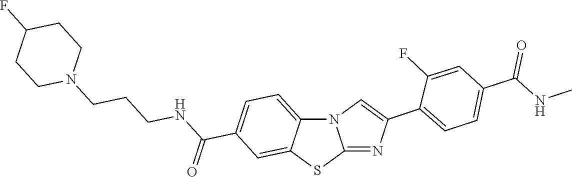 [SMILES: CNC(=O)c1ccc(-c2cn3c(n2)sc2cc(C(=O)NCCCN4CCC(F)CC4)ccc23)c(F)c1] | 2-(2-fluoro-4- (methylcarbamoyl)phenyl)- N-(3-(4-fluoropiperidin-1- yl)propyl)benzo[d]imidazo [2,1-b]thiazole-7-carboxamide |
| 321 | 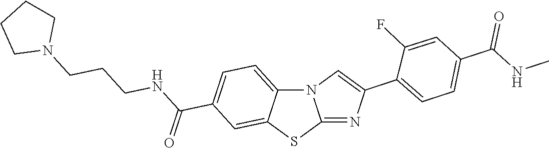 [SMILES: CNC(=O)c1ccc(-c2cn3c(n2)sc2cc(C(=O)NCCCN4CCCC4)ccc23)c(F)c1] | 2-(2-fluoro-4- (methylcarbamoyl)phenyl)- N-(3-(pyrrolidin-1- yl)propyl)benzo[d]imidazo [2,1-b]thiazole-7-carboxamide |
| 322 | 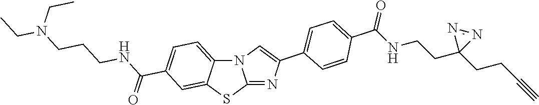 [SMILES: C#CCCC1(CCNC(=O)c2ccc(-c3cn4c(n3)sc3cc(C(=O)NCCCN(CC)CC)ccc34)cc2)N=N1] | 2-(4-((2-(3-(but-3-yn-1-yl)- 3H-diazirin-3- yl)ethyl)carbamoyl)phenyl)- N-(3-(diethylamino)propyl) benzo[d]imidazo[2,1-b] thiazole-7-carboxamide |
| 323 | 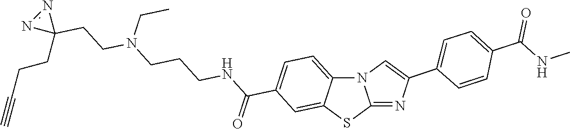 [SMILES: C#CCCC1(CCN(CC)CCCNC(=O)c2ccc3c(c2)sc2nc(-c4ccc(C(=O)NC)cc4)cn23)N=N1] | N-(3-((2-(3-(but-3-yn-1- yl)-3H-diazirin-3- yl)ethyl)(ethyl)amino) propyl)-2-(4- (methylcarbamoyl)phenyl) benzo[d]imidazo[2,1- b]thiazole-7-carboxamide |
| 324 | 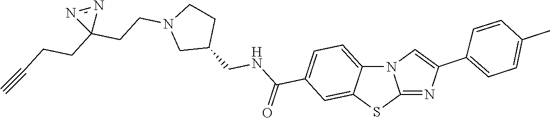 [SMILES: C#CCCC1(CCN2CC[C@H](CNC(=O)c3ccc4c(c3)sc3nc(-c5ccc(C)cc5)cn34)C2)N=N1] | (R)-N-((1-(2-(3-(but-3-yn-1- yl)-3H-diazirin-3- yl)ethyl)pyrrolidin-3- yl)methyl)-2-(p- tolyl)benzo[d]imidazo[2,1- b]thiazole-7-carboxamide |
| 325 | 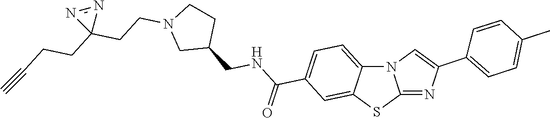 [SMILES: C#CCCC1(CCN2CC[C@@H](CNC(=O)c3ccc4c(c3)sc3nc(-c5ccc(C)cc5)cn34)C2)N=N1] | (S)-N-((1-(2-(3-(but-3-yn-1- yl)-3H-diazirin-3- yl)ethyl)pyrrolidin-3- yl)methyl)-2-(p- tolyl)benzo[d]imidazo[2,1- b]thiazole-7-carboxamide |
| 326 | 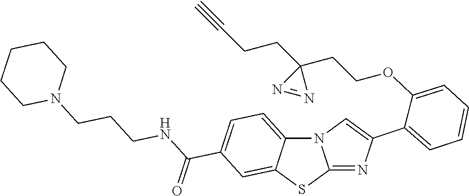 [SMILES: C#CCCC1(CCOc2ccccc2-c2cn3c(n2)sc2cc(C(=O)NCCCN4CCCCC4)ccc23)N=N1] | 2-(2-(2-(3-(but-3-yn-1-yl)- 3H-diazirin-3- yl)ethoxy)phenyl)-N-(3- (piperidin-1- yl)propyl)benzo[d]imidazo [2,1-b]thiazole-7-carboxamide |
| 327 | 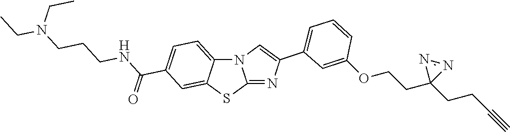 [SMILES: C#CCCC1(CCOc2cccc(-c3cn4c(n3)sc3cc(C(=O)NCCCN(CC)CC)ccc34)c2)N=N1] | 2-(3-(2-(3-(but-3-yn-1-yl)- 3H-diazirin-3- yl)ethoxy)phenyl)-N-(3- (diethylamino)propyl)benzo [d]imidazo[2,1-b]thiazole-7- carboxamide |
| 328 | 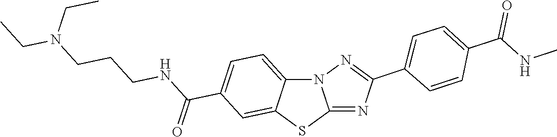 [SMILES: CCN(CC)CCCNC(=O)c1ccc2c(c1)sc1nc(-c3ccc(C(=O)NC)cc3)nn12] | N-(3-(diethylamino)propyl)- 2-(4- (methylcarbamoyl)phenyl) benzo[4,5]thiazolo[3,2- b][1,2,4]triazole-6- carboxamide |
| 329 | 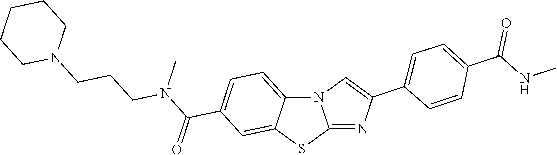 [SMILES: CNC(=O)c1ccc(-c2cn3c(n2)sc2cc(C(=O)N(C)CCCN4CCCCC4)ccc23)cc1] | N-methyl-2-(4- (methylcarbamoyl)phenyl)- N-(3-(piperidin-1- yl)propyl)benzo[d]imidazo [2,1-b]thiazole-7-carboxamide |
| 330 | 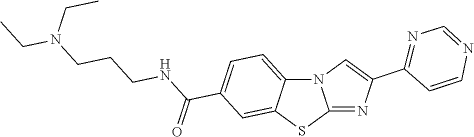 [SMILES: CCN(CC)CCCNC(=O)c1ccc2c(c1)sc1nc(-c3ccncn3)cn12] | N-(3-(diethylamino)propyl)- 2-(pyrimidin-4- yl)benzo[d]imidazo[2,1- b]thiazole-7-carboxamide |
| 331 | 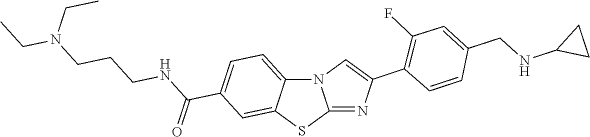 [SMILES: CCN(CC)CCCNC(=O)c1ccc2c(c1)sc1nc(-c3ccc(CNC4CC4)cc3F)cn12] | 2-(4- ((cyclopropylamino)methyl)- 2-fluorophenyl)-N-(3- (diethylamino)propyl)benzo [d]imidazo[2,1-b]thiazole-7- carboxamide |
| 332 | 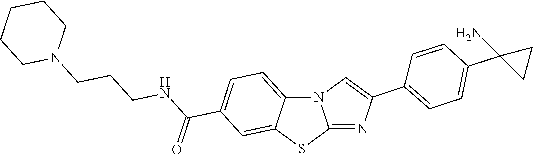 [SMILES: NC1(c2ccc(-c3cn4c(n3)sc3cc(C(=O)NCCCN5CCCCC5)ccc34)cc2)CC1] | 2-(4-(1- aminocyclopropyl)phenyl)- N-(3-(piperidin-1- yl)propyl)benzo[d]imidazo [2,1-b]thiazole-7-carboxamide |
| 333 | 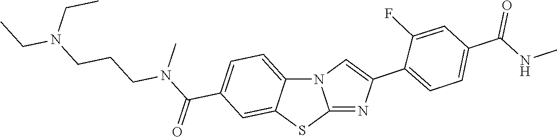 [SMILES: CCN(CC)CCCN(C)C(=O)c1ccc2c(c1)sc1nc(-c3ccc(C(=O)NC)cc3F)cn12] | N-(3-(diethylamino)propyl)- 2-(2-fluoro-4- (methylcarbamoyl)phenyl)- N- methylbenzo[d]imidazo[2,1- b]thiazole-7-carboxamide |
| 334 | 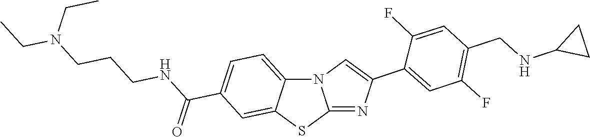 [SMILES: CCN(CC)CCCNC(=O)c1ccc2c(c1)sc1nc(-c3cc(F)c(CNC4CC4)cc3F)cn12] | 2-(4- ((cyclopropylamino)methyl)- 2,5-difluorophenyl)-N-(3- (diethylamino)propyl)benzo [d]imidazo[2,1-b]thiazole-7- carboxamide |
| 335 | 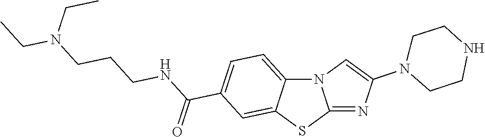 [SMILES: CCN(CC)CCCNC(=O)c1ccc2c(c1)sc1nc(N3CCNCC3)cn12] | N-3-(diethlamino)propyl)- 2-(piperazin-1- yl)benzo[d]imidazo[2,1- b]thiazole-7-carboxamide |
| 336 | 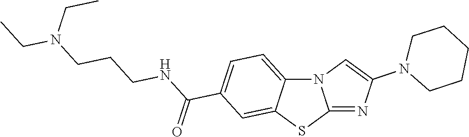 [SMILES: CCN(CC)CCCNC(=O)c1ccc2c(c1)sc1nc(N3CCCCC3)cn12] | N-(3-(diethylamino)propyl)- 2-(piperidin-1- yl)benzo[d]imidazo[2,1- b]thiazole-7-carboxamide |
| 337 | 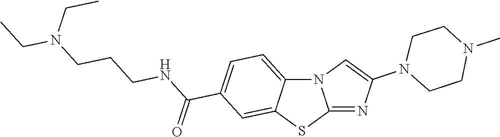 [SMILES: CCN(CC)CCCNC(=O)c1ccc2c(c1)sc1nc(N3CCN(C)CC3)cn12] | N-(3-(diethylamino)propyl)- 2-(4-methylpiperazin-1- yl)benzo[d]imidazo[2,1- b]thiazole-7-carboxamide |
| 338 | 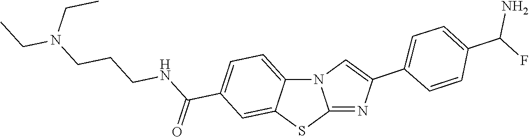 [SMILES: CCN(CC)CCCNC(=O)c1ccc2c(c1)sc1nc(-c3ccc(C(N)F)cc3)cn12] | 2-(4- (aminofluoromethyl)phenyl)- N-(3- (diethylamino)propyl)benzo [d]imidazo[2,1-b]thiazole-7- carboxamide |
| 339 | 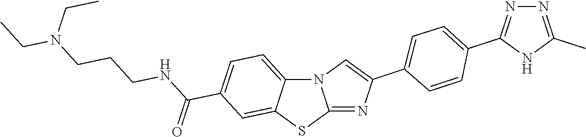 [SMILES: CCN(CC)CCCNC(=O)c1ccc2c(c1)sc1nc(-c3ccc(-c4nnc(C)[nH]4)cc3)cn12] | N-(3-(diethylamino)propyl)- 2-(4-(5-methyl-4H-1,2,4- triazol-3- yl)phenyl)benzo[d]imidazo [2,1-b]thiazole-7-carboxamide |
| 340 | 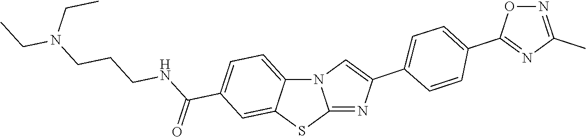 [SMILES: CCN(CC)CCCNC(=O)c1ccc2c(c1)sc1nc(-c3ccc(-c4nc(C)no4)cc3)cn12] | N-(3-(diethylamino)propyl)- 2-(4-(3-methyl-1,2,4- oxadiazol-5- yl)phenyl)benzo[d]imidazo [2,1-b]thiazole-7-carboxamide |
| 341 | 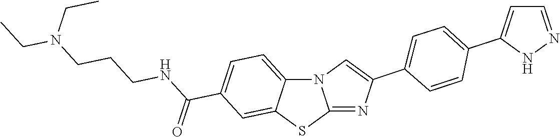 [SMILES: CCN(CC)CCCNC(=O)c1ccc2c(c1)sc1nc(-c3ccc(-c4ccn[nH]4)cc3)cn12] | 2-(4-(1H-pyrazol-5- yl)phenyl)-N-(3- (diethylamino)propyl)benzo [d]imidazo[2,1-b]thiazole-7- carboxamide |
| 342 | 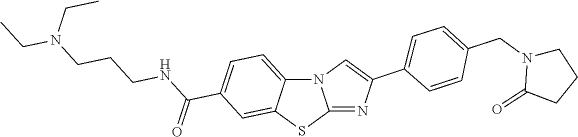 [SMILES: CCN(CC)CCCNC(=O)c1ccc2c(c1)sc1nc(-c3ccc(CN4CCCC4=O)cc3)cn12] | N-(3-(diethylamino)propyl)- 2-(4-((2-oxopyrrolidin-1- yl)methyl)phenyl)benzo[d] imidazo[2,1-b]thiazole-7- carboxamide |
| 343 | 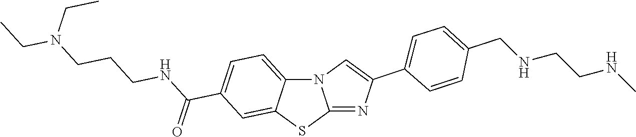 [SMILES: CCN(CC)CCCNC(=O)c1ccc2c(c1)sc1nc(-c3ccc(CNCCNC)cc3)cn12] | N-(3-(diethylamino)propyl)- 2-(4-(((2- (methylamino)ethyl)amino) methyl)phenyl)benzo[d] imidazo[2,1-b]thiazole-7- carboxamide |
| 344 | 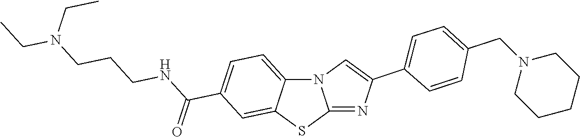 [SMILES: CCN(CC)CCCNC(=O)c1ccc2c(c1)sc1nc(-c3ccc(CN4CCCCC4)cc3)cn12] | N-(3-(diethylamino)propyl)- 2-(4-(piperidin-1- ylmethyl)phenyl)benzo[d] imidazo[2,1-b]thiazole-7- carboxamide |
| 345 | 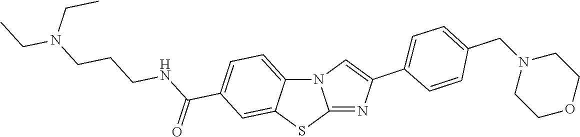 [SMILES: CCN(CC)CCCNC(=O)c1ccc2c(c1)sc1nc(-c3ccc(CN4CCOCC4)cc3)cn12] | N-(3-(diethylamino)propyl)- 2-(4- (morpholinomethyl)phenyl) benzo[d]imidazo[2,1- b]thiazole-7-carboxamide |
| 346 | 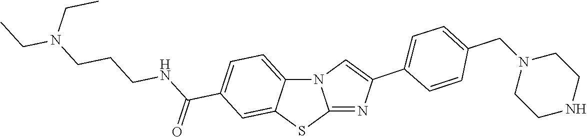 [SMILES: CCN(CC)CCCNC(=O)c1ccc2c(c1)sc1nc(-c3ccc(CN4CCNCC4)cc3)cn12] | N-(3-(diethylamino)propyl)- 2-(4-(piperazin-1- ylmethyl)phenyl)benzo[d] imidazo[2,1-b]thiazole-7- carboxamide |
| 347 | 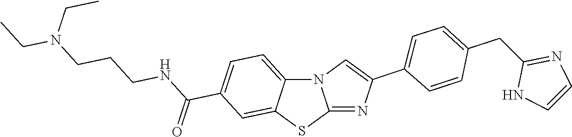 [SMILES: CCN(CC)CCCNC(=O)c1ccc2c(c1)sc1nc(-c3ccc(Cc4ncc[nH]4)cc3)cn12] | 2-(4-((1H-imidazol-2- yl)methyl)phenyl)-N-(3- (diethylamino)propyl)benzo [d]imidazo[2,1-b]thiazole-7- carboxamide |
| 348 | 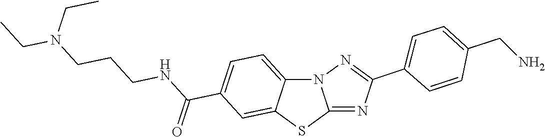 [SMILES: CCN(CC)CCCNC(=O)c1ccc2c(c1)sc1nc(-c3ccc(CN)cc3)nn12] | 2-(4-(aminomethyl)phenyl)- N-(3- (diethylamino)propyl)benzo [4,5]thiazolo[3,2- b][1,2,4]triazole-6- carboxamide |
| 349 | 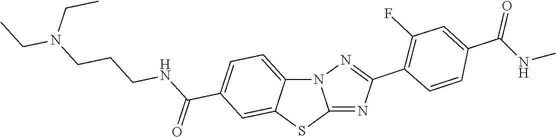 [SMILES: CCN(CC)CCCNC(=O)c1ccc2c(c1)sc1nc(-c3ccc(C(=O)NC)cc3F)nn12] | N-(3-(diethylamino)propyl)- 2-(2-fluoro-4- (methylcarbamoyl)phenyl) benzo[4,5]thiazolo[3,2-b] [1,2,4]triazole-6- carboxamide |
| 350 | 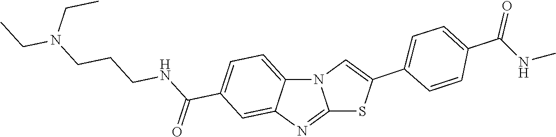 [SMILES: CCN(CC)CCCNC(=O)c1ccc2c(c1)nc1sc(-c3ccc(C(=O)NC)cc3)cn12] | N-(3-(diethylamino)propyl)- 2-(4- (methylcarbamoyl)phenyl) benzo[4,5]imidazo[2,1-b] thiazole-7-carboxamide |
| 351 | 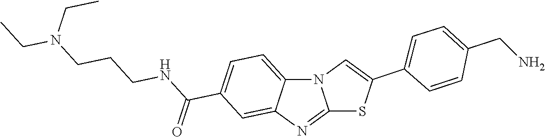 [SMILES: CCN(CC)CCCNC(=O)c1ccc2c(c1)nc1sc(-c3ccc(CN)cc3)cn12] | 2-(4-(aminomethyl)phenyl)- N-(3- (diethylamino)propyl)benzo [4,5]imidazo[2,1-b]thiazole- 7-carboxamide |
| 352 | 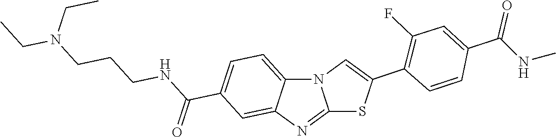 [SMILES: CCN(CC)CCCNC(=O)c1ccc2c(c1)nc1sc(-c3ccc(C(=O)NC)cc3F)cn12] | N-(3-(diethylamino)propyl)- 2-(2-fluoro-4- (methylcarbamoyl)phenyl) benzo[4,5]imidazo[2,1- b]thiazole-7-carboxamide |
| 353 | 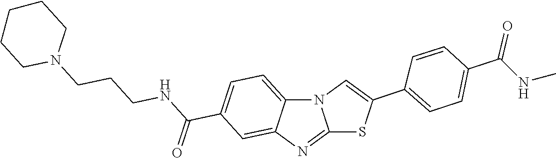 [SMILES: CNC(=O)c1ccc(-c2cn3c(nc4cc(C(=O)NCCCN5CCCCC5)ccc43)s2)cc1] | 2-(4- (methylcarbamoyl)phenyl) N-(3-(piperidin-1- yl)propyl)benzo[4,5]imidazo [2,1-b]thiazole-7- carboxamide |
| 354 | 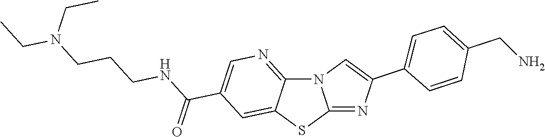 [SMILES: CCN(CC)CCCNC(=O)c1cnc2c(c1)sc1nc(-c3ccc(CN)cc3)cn12] | 2-(4-(aminomethyl)phenyl)- N-(3- (diethylamino)propyl)imidazo [2′,1′:2,3]thiazolo[4,5-b] pyridine-7-carboxamide |
| 355 | 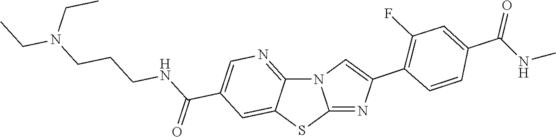 [SMILES: CCN(CC)CCCNC(=O)c1cnc2c(c1)sc1nc(-c3ccc(C(=O)NC)cc3F)cn12] | N-(3-(diethylamino)propyl)- 2-(2-fluoro-4- (methylcarbamoyl)phenyl) imidazo[2′,1′:2,3]thiazolo[4,5- b]pyridine-7-carboxamide |
| 356 | 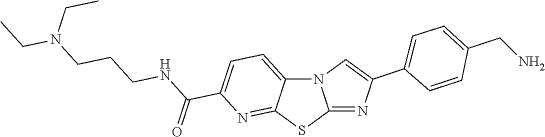 [SMILES: CCN(CC)CCCNC(=O)c1ccc2c(n1)sc1nc(-c3ccc(CN)cc3)cn12] | 2-(4-(aminomethyl)phenyl)- N-(3- (diethylamino)propyl) imidazo[2′,1′:2,3]thiazolo[5,4- b]pyridine-7-carboxamide |
| 357 | 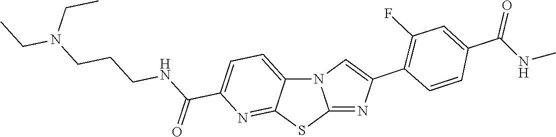 [SMILES: CCN(CC)CCCNC(=O)c1ccc2c(n1)sc1nc(-c3ccc(C(=O)NC)cc3F)cn12] | N-(3-(diethylamino)propyl)- 2-(2-fluoro-4- (methylcarbamoyl)phenyl) imidazo[2′,1′:2,3]thiazolo[5,4- b]pyridine-7-carboxamide |
| 358 | 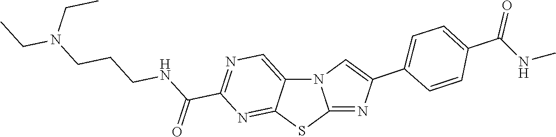 [SMILES: CCN(CC)CCCNC(=O)c1ncc2c(n1)sc1nc(-c3ccc(C(=O)NC)cc3)cn12] | N-(3-(diethylamino)propyl)- 7-(4- (methylcarbamoyl)phenyl) imidazo[2′,1′:2,3]thiazolo[5,4- d]pyrimidine-2-carboxamide |
| 359 | 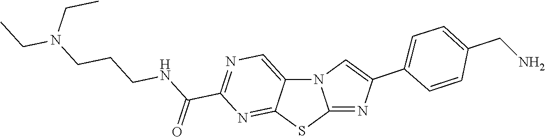 [SMILES: CCN(CC)CCCNC(=O)c1ncc2c(n1)sc1nc(-c3ccc(CN)cc3)cn12] | 7-(4-(aminomethyl)phenyl)- N-(3- (diethylamino)propyl)imidazo [2′,1′:2,3]thiazolo[5,4-d] pyrimidine-2-carboxamide |
| 360 | 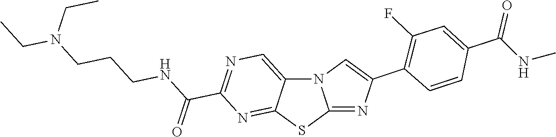 [SMILES: CCN(CC)CCCNC(=O)c1ncc2c(n1)sc1nc(-c3ccc(C(=O)NC)cc3F)cn12] | N-(3-(diethylamino)propyl)- 7-(2-fluoro-4- (methylcarbamoyl)phenyl) imidazo[2′,1′:2,3]thiazolo[5,4- d]pyrimidine-2-carboxamide |
| 361 | 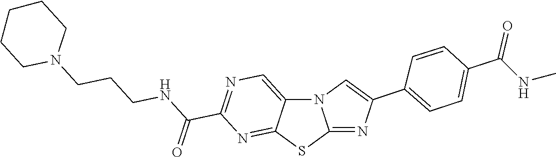 [SMILES: CNC(=O)c1ccc(-c2cn3c(n2)sc2nc(C(=O)NCCCN4CCCCC4)ncc23)cc1] | 7-(4- (methylcarbamoyl)phenyl)- N-(3-(piperidin-1- yl)propyl)imidazo[2′,1′:2,3] thiazolo[5,4-d]pyrimidine-2- carboxamide |
| 362 | 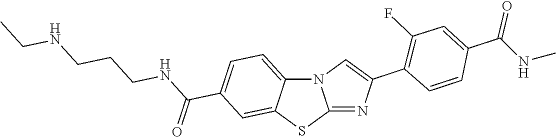 [SMILES: CCNCCCNC(=O)c1ccc2c(c1)sc1nc(-c3ccc(C(=O)NC)cc3F)cn12] | N-(3-(ethylamino)propyl)-2- (2-fluoro-4- (methylcarbamoyl)phenyl) benzo[d]limidazo[2,1- b]thiazole-7-carboxamide |
| 363 | 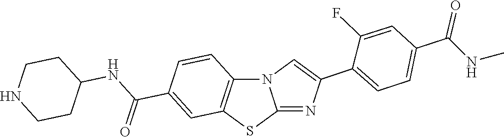 [SMILES: CNC(=O)c1ccc(-c2cn3c(n2)sc2cc(C(=O)NC4CCNCC4)ccc23)c(F)c1] | 2-(2-fluoro-4- (methylcarbamoyl)phenyl)- N-(piperidin-4- yl)benzo[d]imidazo[2,1- b]thiazole-7-carboxamide |
| 364 | 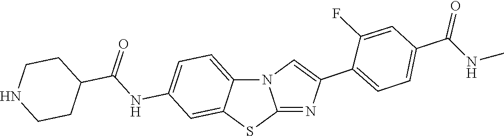 [SMILES: CNC(=O)c1ccc(-c2cn3c(n2)sc2cc(NC(=O)C4CCNCC4)ccc23)c(F)c1] | N-(2-(2-fluoro-4- (methylcarbamoyl)phenyl) benzo[d]imidazo[2,1-b]thiazol- 7-yl)piperidine-4- carboxamide |
| 365 | 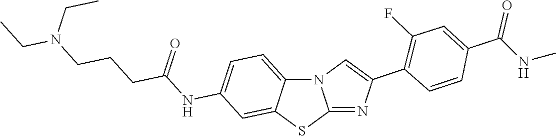 [SMILES: CCN(CC)CCCC(=O)Nc1ccc2c(c1)sc1nc(-c3ccc(C(=O)NC)cc3F)cn12] | 4-(7-(4- (diethylamino)butanamido) benzo[d]imidazo[2,1- b]thiazol-2-yl)-3-fluoro-N- methylbenzamide |
| 366 | 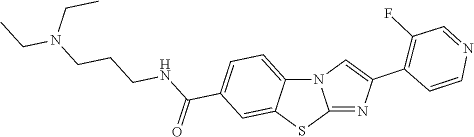 [SMILES: CCN(CC)CCCNC(=O)c1ccc2c(c1)sc1nc(-c3ccncc3F)cn12] | N-(3-(diethylamino)propyl)- 2-(3-fluoropyridin-4- yl)benzo[d]imidazo[2,1- b]thiazole-7-carboxamide |
| 367 | 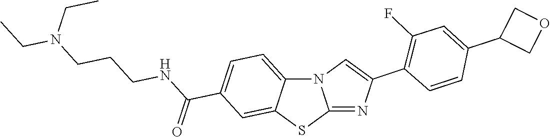 [SMILES: CCN(CC)CCCNC(=O)c1ccc2c(c1)sc1nc(-c3ccc(C4COC4)cc3F)cn12] | N-(3-(diethylamino)propyl)- 2-(2-fluoro-4-(oxetan-3- yl)phenyl)benzo[d]imidazo [2,1-b]thiazole-7-carboxamide |
| 368 | 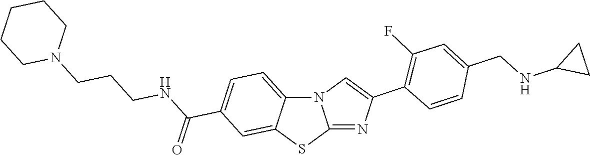 [SMILES: O=C(NCCCN1CCCCC1)c1ccc2c(c1)sc1nc(-c3ccc(CNC4CC4)cc3F)cn12] | 2-(4- ((cyclopropylamino)methyl)- 2-fluorophenyl)-N-(3- (piperidin-1- yl)propyl)benzo[d]imidazo [2,1-b]thiazole-7-carboxamide |
| 369 | 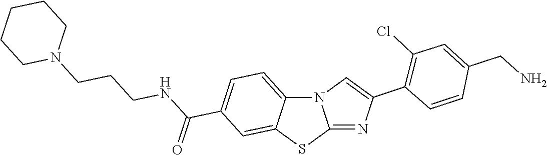 [SMILES: NCc1ccc(-c2cn3c(n2)sc2cc(C(=O)NCCCN4CCCCC4)ccc23)c(Cl)c1] | 2-(4-(aminomethyl)-2- chlorophenyl)-N-(3- (piperidin-1- yl)propyl)benzo[d]imidazo [2,1-b]thiazole-7-carboxamide |
| 370 | 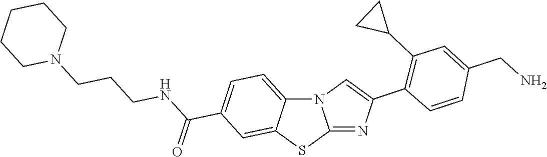 [SMILES: NCc1ccc(-c2cn3c(n2)sc2cc(C(=O)NCCCN4CCCCC4)ccc23)c(C2CC2)c1] | 2-(4-(aminomethyl)-2- cyclopropylphenyl)-N-(3- (piperidin-1- yl)propyl)benzo[d]imidazo [2,1-b]thiazole-7-carboxamide |
| 371 | 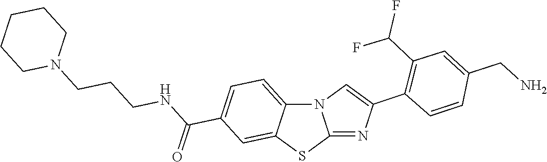 [SMILES: NCc1ccc(-c2cn3c(n2)sc2cc(C(=O)NCCCN4CCCCC4)ccc23)c(C(F)F)c1] | 2-(4-(aminomethyl)-2- (difluoromethyl)phenyl)-N- (3-(piperidin-1- yl)propyl)benzo[d]imidazo [2,1-b]thiazole-7-carboxamide |
| 372 | 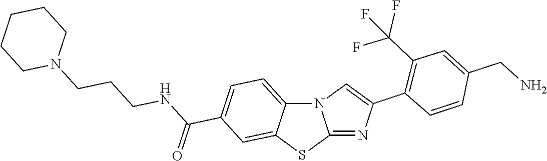 [SMILES: NCc1ccc(-c2cn3c(n2)sc2cc(C(=O)NCCCN4CCCCC4)ccc23)c(C(F)(F)F)c1] | 2-(4-(aminomethyl)-2- (trifluoromethyl)phenyl)-N- (3-(piperidin-1- yl)propyl)benzo[d]imidazo [2,1-b]thiazole-7-carboxamide |
| 373 | 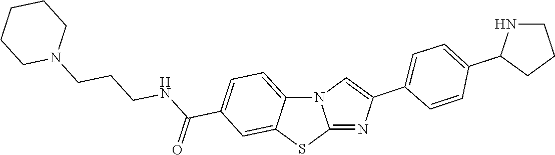 [SMILES: O=C(NCCCN1CCCCC1)c1ccc2c(c1)sc1nc(-c3ccc(C4CCCN4)cc3)cn12] | N-(3-(piperidin-1-yl)propyl)- 2-(4-(pyrrolidin-2- yl)phenyl)benzo[d]imidazo [2,1-b]thiazole-7-carboxamide |
| 374 | 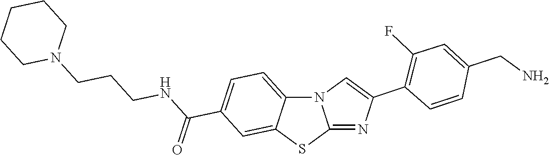 [SMILES: NCc1ccc(-c2cn3c(n2)sc2cc(C(=O)NCCCN4CCCCC4)ccc23)c(F)c1] | 2-(4-(aminomethyl)-2- fluorophenyl)-N-(3- (piperidin-1- yl)propyl)benzo[d]imidazo [2,1-b]thiazole-7-carboxamide |
| 375 | 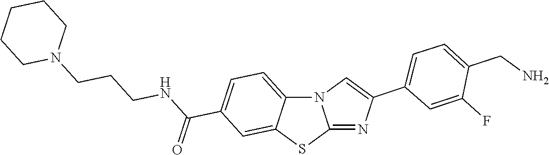 [SMILES: NCc1ccc(-c2cn3c(n2)sc2cc(C(=O)NCCCN4CCCCC4)ccc23)cc1F] | 2-(4-(aminomethyl)-3- fluorophenyl)-N-(3- (piperidin-1- yl)propyl)benzo[d]imidazo [2,1-b]thiazole-7-carboxamide |
| 376 | 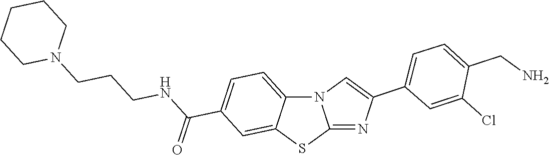 [SMILES: NCc1ccc(-c2cn3c(n2)sc2cc(C(=O)NCCCN4CCCCC4)ccc23)cc1Cl] | 2-(4-(aminomethyl)-3- chlorophenyl)-N-(3- (piperidin-1- yl)propyl)benzo[d]imidazo [2,1-b]thiazole-7-carboxamide |
| 377 | 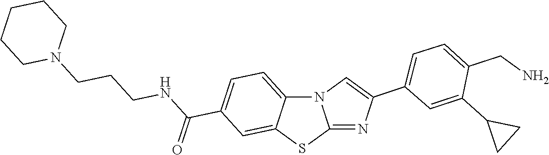 [SMILES: NCc1ccc(-c2cn3c(n2)sc2cc(C(=O)NCCCN4CCCCC4)ccc23)cc1C1CC1] | 2-(4-(aminomethyl)-3- cyclopropylphenyl)-N- (3-(piperidin-1- yl)propyl)benzo[d]imidazo [2,1-b]thiazole-7-carboxamide |
| 378 | 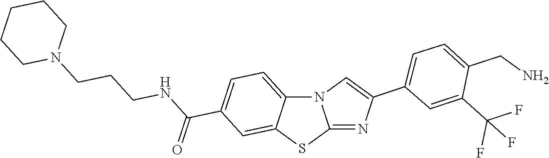 [SMILES: NCc1ccc(-c2cn3c(n2)sc2cc(C(=O)NCCCN4CCCCC4)ccc23)cc1C(F)(F)F] | 2-(4-(aminomethyl)-3- (trifluoromethyl)phenyl)- N-(3-(piperidin-1- yl)propyl)benzo[d]imidazo [2,1-b]thiazole-7-carboxamide |
| 379 | 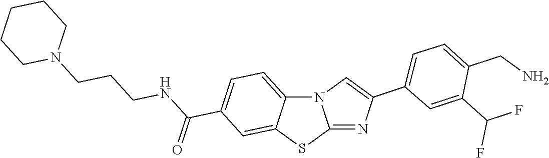 [SMILES: NCc1ccc(-c2cn3c(n2)sc2cc(C(=O)NCCCN4CCCCC4)ccc23)cc1C(F)F] | 2-(4-(aminomethyl)-3- (difluoromethyl)phenyl)-N- (3-(piperidin-1- yl)propyl)benzo[d]imidazo [2,1-b]thiazole-7-carboxamide |
| 380 | 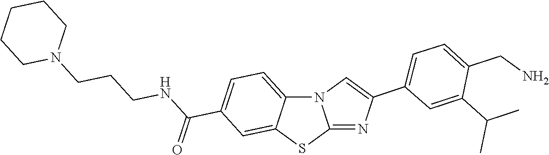 [SMILES: CC(C)c1cc(-c2cn3c(n2)sc2cc(C(=O)NCCCN4CCCCC4)ccc23)ccc1CN] | 2-(4-(aminomethyl)-3- isopropylphenyl)-N-(3- (piperidin-1- yl)propyl)benzo[d]imidazo [2,1-b]thiazole-7-carboxamide |
| 381 | 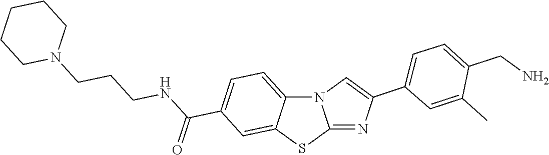 [SMILES: Cc1cc(-c2cn3c(n2)sc2cc(C(=O)NCCCN4CCCCC4)ccc23)ccc1CN] | 2-(4-(aminomethyl)-3- methylphenyl)-N-(3- (piperidin-1- yl)propyl)benzo[d]imidazo [2,1-b]thiazole-7-carboxamide |
| 382 | 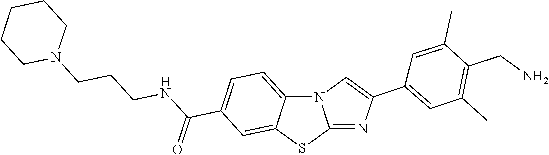 [SMILES: Cc1cc(-c2cn3c(n2)sc2cc(C(=O)NCCCN4CCCCC4)ccc23)cc(C)c1CN] | 2-(4-(aminomethyl)-3,5- dimethylphenyl)-N-(3- (piperidin-1- yl)propyl)benzo[d]imidazo [2,1-b]thiazole-7-carboxamide |
| 383 | 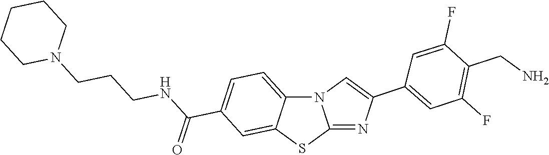 [SMILES: NCc1c(F)cc(-c2cn3c(n2)sc2cc(C(=O)NCCCN4CCCCC4)ccc23)cc1F] | 2-(4-(aminomethyl)-3,5- difluorophenyl)-N-(3- (piperidin-1- yl)propyl)benzo[d]imidazo [2,1-b]thiazole-7-carboxamide |
| 384 | 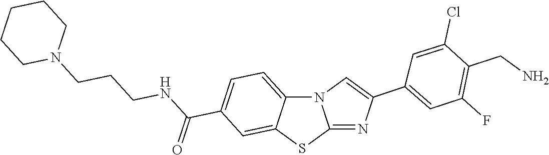 [SMILES: NCc1c(F)cc(-c2cn3c(n2)sc2cc(C(=O)NCCCN4CCCCC4)ccc23)cc1Cl] | 2-(4-(aminomethyl)-3- chloro-5-fluorophenyl)-N-(3- (piperidin-1- yl)propyl)benzo[d]imidazo [2,1-b]thiazole-7-carboxamide |
| 385 | 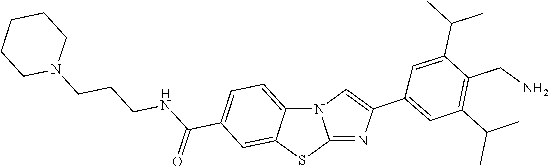 [SMILES: CC(C)c1cc(-c2cn3c(n2)sc2cc(C(=O)NCCCN4CCCCC4)ccc23)cc(C(C)C)c1CN] | 2-(4-(aminomethyl)-3,5- diisopropylphenyl)-N-(3- (piperidin-1- yl)propyl)benzo[d]imidazo [2,1-b]thiazole-7-carboxamide |
| 386 | 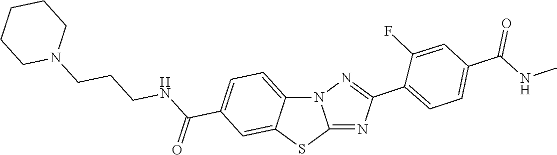 [SMILES: CNC(=O)c1ccc(-c2nc3sc4cc(C(=O)NCCCN5CCCCC5)ccc4n3n2)c(F)c1] | 2-(2-fluoro-4- (methylcarbamoyl)phenyl)- N-(3-(piperidin-1- yl)propyl)benzo[4,5]thiazolo [3,2-b][1,2,4]triazole-6- carboxamide |
| 387 | 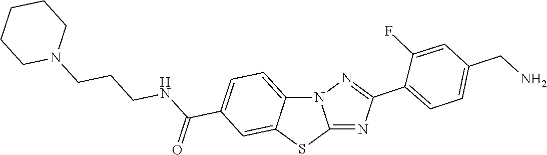 [SMILES: NCc1ccc(-c2nc3sc4cc(C(=O)NCCCN5CCCCC5)ccc4n3n2)c(F)c1] | 2-(4-(aminomethyl)-2- fluorophenyl)-N-(3- (piperidin-1- yl)propyl)benzo[4,5]thiazolo [3,2-b][1,2,4]triazole-6- carboxamide |
| 388 | 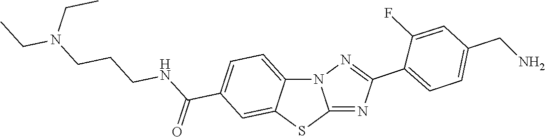 [SMILES: CCN(CC)CCCNC(=O)c1ccc2c(c1)sc1nc(-c3ccc(CN)cc3F)nn12] | 2-(4-(aminomethyl)-2- fluorophenyl)-N-(3- (diethylamino)propyl) benzo[4,5]thiazolo[3,2- b][1,2,4]triazole-6- carboxamide |
| 389 | 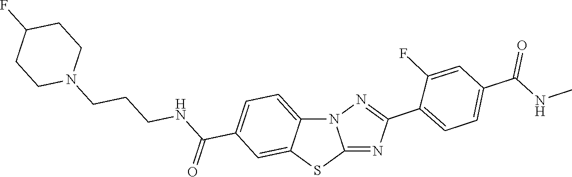 [SMILES: CNC(=O)c1ccc(-c2nc3sc4cc(C(=O)NCCCN5CCC(F)CC5)ccc4n3n2)c(F)c1] | 2-(2-fluoro-4- (methylcarbamoyl)phenyl)- N-(3-(4-fluoropiperidin-1- yl)propyl)benzo[4,5]thiazolo [3,2-b][1,2,4]triazole-6- carboxamide |
| 390 | 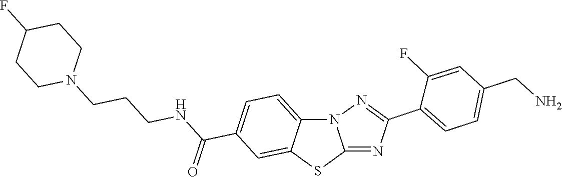 [SMILES: NCc1ccc(-c2nc3sc4cc(C(=O)NCCCN5CCC(F)CC5)ccc4n3n2)c(F)c1] | 2-(4-(aminomethyl)-2- fluorophenyl)-N-(3-(4- fluoropiperidin-1- yl)propyl)benzo[4,5]thiazolo [3,2-b][1,2,4]triazole-6- carboxamide |
| 391 | 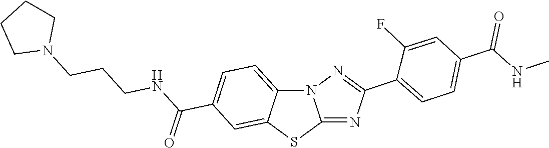 [SMILES: CNC(=O)c1ccc(-c2nc3sc4cc(C(=O)NCCCN5CCCC5)ccc4n3n2)c(F)c1] | 2-(2-fluoro-4- (methylcarbamoyl)phenyl)- N-(3-(pyrrolidin-1- yl)propyl)benzo[4,5]thiazolo [3,2-b][1,2,4]triazole-6- carboxamide |
| 392 | 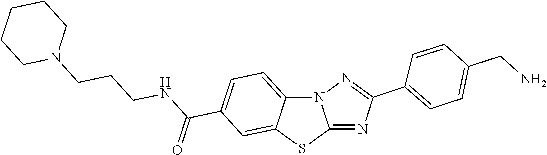 [SMILES: NCc1ccc(-c2nc3sc4cc(C(=O)NCCCN5CCCCC5)ccc4n3n2)cc1] | 2-(4-(aminomethyl)phenyl)- N-(3-(piperidin-1- yl)propyl)benzo[4,5]thiazolo [3,2-b][1,2,4]triazole-6- carboxamide |
| 393 | 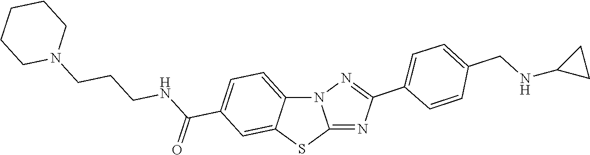 [SMILES: O=C(NCCCN1CCCCC1)c1ccc2c(c1)sc1nc(-c3ccc(CNC4CC4)cc3)nn12] | 2-(4- ((cyclopropylamino)methyl) phenyl)-N-(3-(piperidin-1- yl)propyl)benzo[4,5]thiazolo [3,2-b][1,2,4]triazole-6- carboxamide |
| 394 | 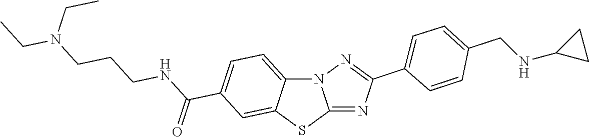 [SMILES: CCN(CC)CCCNC(=O)c1ccc2c(c1)sc1nc(-c3ccc(CNC4CC4)cc3)nn12] | 2-(4- ((cyclopropylamino)methyl) phenyl)-N-(3- (diethylamino)propyl)benzo [4,5]thiazolo[3,2- b][1,2,4]triazole-6- carboxamide |
| 395 | 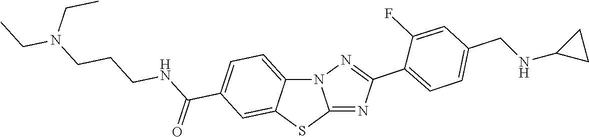 [SMILES: CCN(CC)CCCNC(=O)c1ccc2c(c1)sc1nc(-c3ccc(CNC4CC4)cc3F)nn12] | 2-(4- ((cyclopropylamino)methyl)- 2-fluorophenyl)-N-(3- (diethylamino)propyl)benzo [4,5]thiazolo[3,2- b][1,2,4]triazole-6- carboxamide |
| 396 | 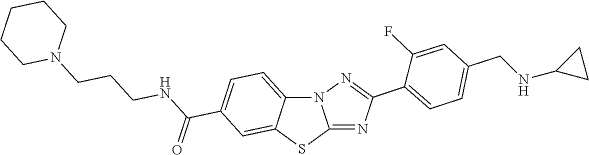 [SMILES: O=C(NCCCN1CCCCC1)c1ccc2c(c1)sc1nc(-c3ccc(CNC4CC4)cc3F)nn12] | 2-(4- ((cyclopropylamino)methyl)- 2-fluorophenyl)-N-(3- (piperidin-1- yl)propyl)benzo[4,5]thiazolo [3,2-b][1,2,4]triazole-6- carboxamide |
| 397 | 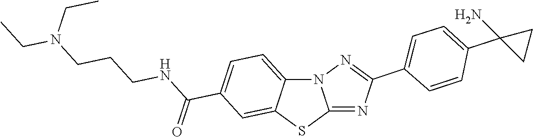 [SMILES: CCN(CC)CCCNC(=O)c1ccc2c(c1)sc1nc(-c3ccc(C4(N)CC4)cc3)nn12] | 2-(4-(1- aminocyclopropyl)phenyl)- N-(3- (diethylamino)propyl)benzo [4,5]thiazolo[3,2- b][1,2,4]triazole-6- carboxamide |
| 398 | 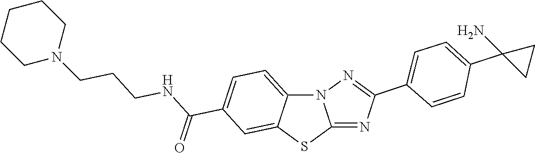 [SMILES: NC1(c2ccc(-c3nc4sc5cc(C(=O)NCCCN6CCCCC6)ccc5n4n3)cc2)CC1] | 2-(4-(1- aminocyclopropyl)phenyl)- N-(3-(piperidin-1- yl)propyl)benzo[4,5]thiazolo [3,2-b][1,2,4]triazole-6- carboxamide |
| 399 | 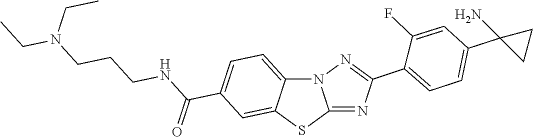 [SMILES: CCN(CC)CCCNC(=O)c1ccc2c(c1)sc1nc(-c3ccc(C4(N)CC4)cc3F)nn12] | 2-(4-(1-aminocyclopropyl)- 2-fluorophenyl)-N-(3- (diethylamino)propyl)benzo [4,5]thiazolo[3,2- b][1,2,4]triazole-6- carboxamide |
| 400 | 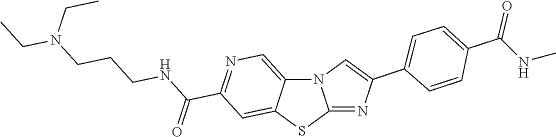 [SMILES: CCN(CC)CCCNC(=O)c1cc2sc3nc(-c4ccc(C(=O)NC)cc4)cn3c2cn1] | N-(3-(diethylamino)propyl)- 2-(4- (methylcarbamoyl)phenyl) imidazo[2′,1′:2,3]thiazolo[4,5- c]pyridine-7-carboxamide |
| 401 | 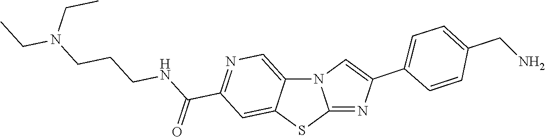 [SMILES: CCN(CC)CCCNC(=O)c1cc2sc3nc(-c4ccc(CN)cc4)cn3c2cn1] | 2-(4-(aminomethyl)phenyl)- N-(3- (diethylamino)propyl) imidazo[2′,1′:2,3]thiazolo[4,5- c]pyridine-7-carboxamide |
| 402 | 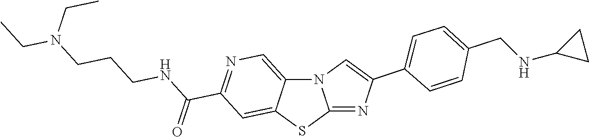 [SMILES: CCN(CC)CCCNC(=O)c1cc2sc3nc(-c4ccc(CNC5CC5)cc4)cn3c2cn1] | 2-(4- ((cyclopropylamino)methyl) phenyl)-N-(3- (diethylamino)propyl) imidazo[2′,1′:2,3]thiazolo [4,5-c]pyridine-7-carboxamide |
| 403 | 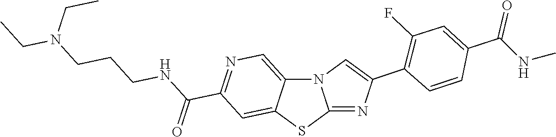 [SMILES: CCN(CC)CCCNC(=O)c1cc2sc3nc(-c4ccc(C(=O)NC)cc4F)cn3c2cn1] | N-(3-(diethylamino)propyl)- 2-(2-fluoro-4- (methylcarbamoyl)phenyl) imidazo[2′,1′:2,3]thiazolo[4,5- c]pyridine-7-carboxamide |
| 404 | 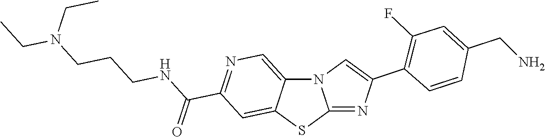 [SMILES: CCN(CC)CCCNC(=O)c1cc2sc3nc(-c4ccc(CN)cc4F)cn3c2cn1] | 2-(4-(aminomethyl)-2- fluorophenyl)-N-(3- (diethylamino)propyl) imidazo[2′,1′:2,3]thiazolo[4,5- c]pyridine-7-carboxamide |
| 405 | 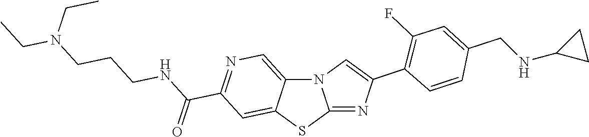 [SMILES: CCN(CC)CCCNC(=O)c1cc2sc3nc(-c4ccc(CNC5CC5)cc4F)cn3c2cn1] | 2-(4- ((cyclopropylamino)methyl)- 2-fluorophenyl)-N-(3- (diethylamino)propyl) imidazo[2′1′:2,3]thiazolo[4,5- c]pyridine-7-carboxamide |
| 406 | 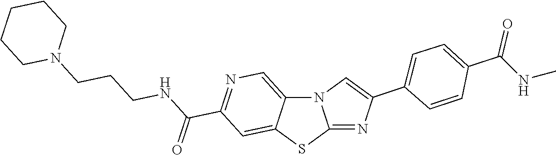 [SMILES: CNC(=O)c1ccc(-c2cn3c(n2)sc2cc(C(=O)NCCCN4CCCCC4)ncc23)cc1] | 2-(4- (methylcarbamoyl)phenyl)- N-(3-(piperidin-1- yl)propyl)imidazo[2′,1′:2,3] thiazolo[4,5-c]pyridine-7- carboxamide |
| 407 | 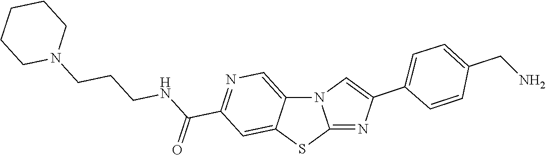 [SMILES: NCc1ccc(-c2cn3c(n2)sc2cc(C(=O)NCCCN4CCCCC4)ncc23)cc1] | 2-(4-(aminomethyl)phenyl)- N-(3-(piperidin-1- yl)propyl)imidazo[2′,1′:2,3] thiazolo[4,5-c]pyridine-7- carboxamide |
| 408 | 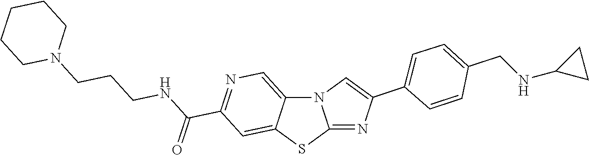 [SMILES: O=C(NCCCN1CCCCC1)c1cc2sc3nc(-c4ccc(CNC5CC5)cc4)cn3c2cn1] | 2-(4- ((cyclopropylamino)methyl) phenyl)-N-(3-(piperidin-1- yl)propyl)imidazo[2′,1′:2,3] thiazolo[4,5-c]pyridine-7- carboxamide |
| 409 | 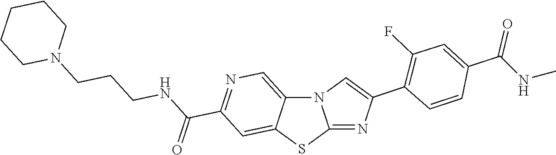 [SMILES: CNC(=O)c1ccc(-c2cn3c(n2)sc2cc(C(=O)NCCCN4CCCCC4)ncc23)c(F)c1] | 2-(2-fluoro-4- (methylcarbamoyl)phenyl)- N-(3-(piperidin-1- yl)propyl)imidazo[2′,1′:2,3] thiazolo[4,5-c]pyridine-7- carboxamide |
| 410 | 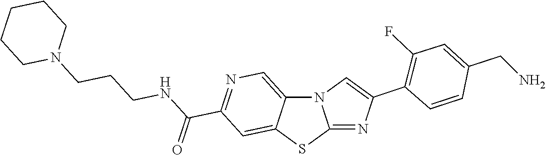 [SMILES: NCc1ccc(-c2cn3c(n2)sc2cc(C(=O)NCCCN4CCCCC4)ncc23)c(F)c1] | 2-(4-(aminomethyl)-2- fluorophenyl)-N-(3- (piperidin-1- yl)propyl)imidazo[2′,1′:2,3] thiazolo[4,5-c]pyridine-7- carboxamide |
| 411 | 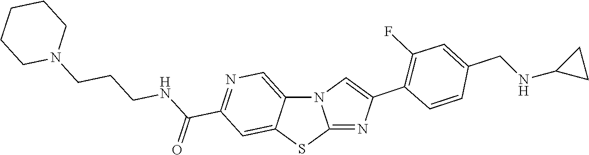 [SMILES: O=C(NCCCN1CCCCC1)c1cc2sc3nc(-c4ccc(CNC5CC5)cc4F)cn3c2cn1] | 2-(4- ((cyclopropylamino)methyl)- 2-fluorophenyl)-N-(3- (piperidin-1- yl)propyl)imidazo[2′,1′:2,3] thiazolo[4,5-c]pyridine-7- carboxamide |
| 412 | 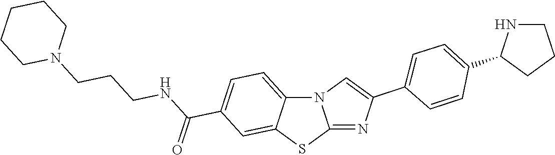 [SMILES: O=C(NCCCN1CCCCC1)c1ccc2c(c1)sc1nc(-c3ccc([C@H]4CCCN4)cc3)cn12] | (R)-N-(3-(piperidin-1- yl)propyl)-2-(4-(pyrrolidin- 2- yl)phenyl)benzo[d]imidazo [2,1-b]thiazole-7-carboxamide |
| 413 | 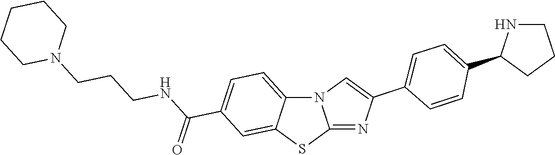 [SMILES: O=C(NCCCN1CCCCC1)c1ccc2c(c1)sc1nc(-c3ccc([C@@H]4CCCN4)cc3)cn12] | (S)-N-(3-(piperidin-1- yl)propyl)-2-(4-(pyrrolidin- 2- yl)phenyl)benzo[d]imidazo [2,1-b]thiazole-7-carboxamide |
| 414 | 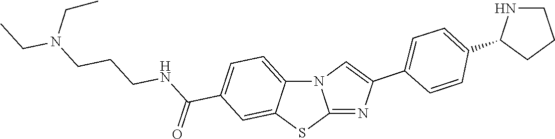 [SMILES: CCN(CC)CCCNC(=O)c1ccc2c(c1)sc1nc(-c3ccc([C@H]4CCCN4)cc3)cn12] | (R)-N-(3- (diethylamino)propyl)-2-(4- (pyrrolidin-2- yl)phenyl)benzo[d]imidazo [2,1-b]thiazole-7-carboxamide |
| 415 | 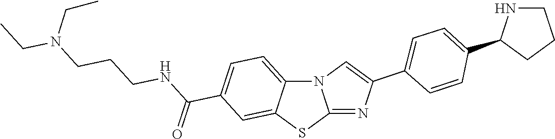 [SMILES: CCN(CC)CCCNC(=O)c1ccc2c(c1)sc1nc(-c3ccc([C@@H]4CCCN4)cc3)cn12] | (S)-N-(3- (diethylamino)propyl)-2-(4- (pyrrolidin-2- yl)phenyl)benzo[d]imidazo [2,1-b]thiazole-7-carboxamide |
| 416 | 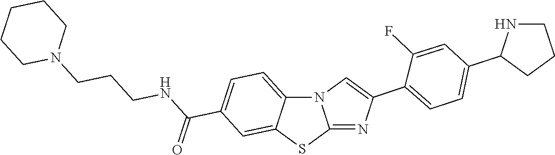 [SMILES: O=C(NCCCN1CCCCC1)c1ccc2c(c1)sc1nc(-c3ccc(C4CCCN4)cc3F)cn12] | 2-(2-fluoro-4-(pyrrolidin-2- yl)phenyl)-N-(3-(piperidin-1- yl)propyl)benzo[d]imidazo [2,1-b]thiazole-7-carboxamide |
| 417 | 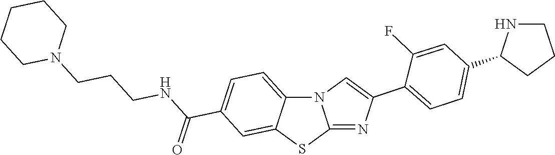 [SMILES: O=C(NCCCN1CCCCC1)c1ccc2c(c1)sc1nc(-c3ccc([C@H]4CCCN4)cc3F)cn12] | (R)-2-(2-fluoro-4- (pyrrolidin-2-yl)phenyl)-N- (3-(piperidin-1- yl)propyl)benzo[d]imidazo [2,1-b]thiazole-7-carboxamide |
| 418 | 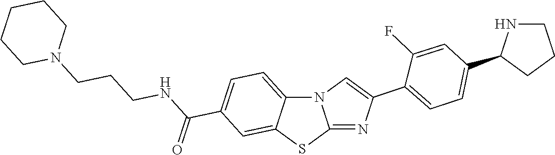 [SMILES: O=C(NCCCN1CCCCC1)c1ccc2c(c1)sc1nc(-c3ccc([C@@H]4CCCN4)cc3F)cn12] | (S)-2-(2-fluoro-4-(pyrrolidin- 2-yl)phenyl)-N-(3-(piperidin- 1- yl)propyl)benzo[d]imidazo [2,1-b]thiazole-7-carboxamide |
| 419 | 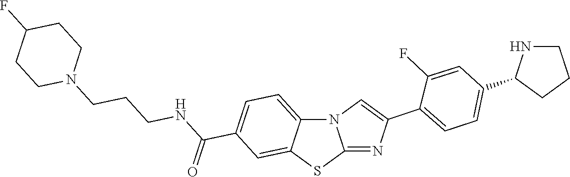 [SMILES: O=C(NCCCN1CCC(F)CC1)c1ccc2c(c1)sc1nc(-c3ccc([C@H]4CCCN4)cc3F)cn12] | (R)-2-(2-fluoro-4- (pyrrolidin-2-yl)phenyl)-N- (3-(4-fluoropiperidin-1- yl)propyl)benzo[d]imidazo [2,1-b]thiazole-7-carboxamide |
| 420 | 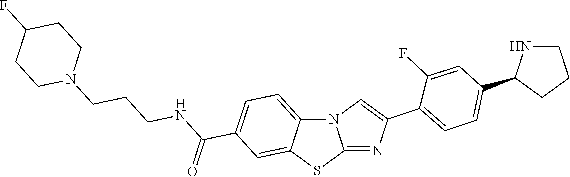 [SMILES: O=C(NCCCN1CCC(F)CC1)c1ccc2c(c1)sc1nc(-c3ccc([C@@H]4CCCN4)cc3F)cn12] | (S)-2-(2-fluoro-4-(pyrrolidin- 2-yl)phenyl)-N-(3-(4- fluoropiperidin-1- yl)propyl)benzo[d]imidazo [2,1-b]thiazole-7-carboxamide |
| 421 | 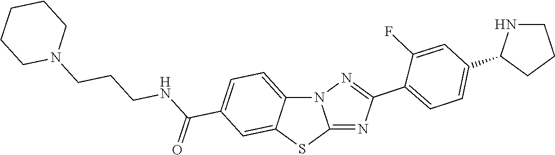 [SMILES: O=C(NCCCN1CCCCC1)c1ccc2c(c1)sc1nc(-c3ccc([C@H]4CCCN4)cc3F)nn12] | (R)-2-(2-fluoro-4- (pyrrolidin-2-yl)phenyl)-N- (3-(piperidin-1- yl)propyl)benzo[4,5]thiazolo [3,2-b][1,2,4]triazole-6- carboxamide |
| 422 | 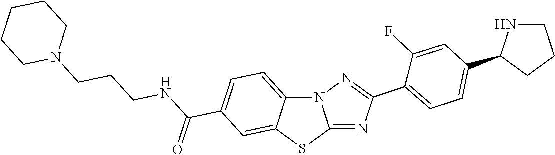 [SMILES: O=C(NCCCN1CCCCC1)c1ccc2c(c1)sc1nc(-c3ccc([C@@H]4CCCN4)cc3F)nn12] | (S)-2-(2-fluoro-4-(pyrrolidin- 2-yl)phenyl)-N-(3-(piperidin- 1- yl)propyl)benzo[4,5]thiazolo [3,2-b][1,2,4]triazole-6- carboxamide |
| 423S | 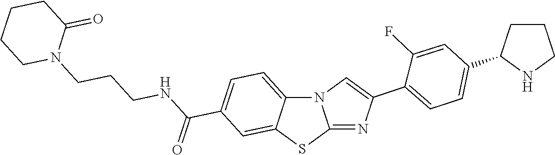 [SMILES: O=C(NCCCN1CCCCC1=O)c1ccc2c(c1)sc1nc(-c3ccc([C@@H]4CCCN4)cc3F)cn12] | (S)-2-(2-fluoro-4-(pyrrolidin- 2-yl)phenyl)-N-(3-(2- oxopiperidin-1- yl)propyl)benzo[d]imidazo [2,1-b]othiazole-7-carboxamide |
| 423R | 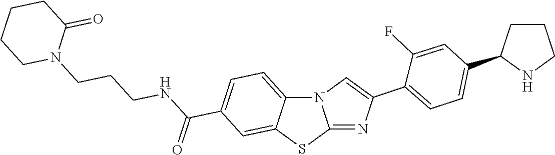 [SMILES: O=C(NCCCN1CCCCC1=O)c1ccc2c(c1)sc1nc(-c3ccc([C@H]4CCCN4)cc3F)cn12] | (R)-2-(2-fluoro-4- (pyrrolidin-2-yl)phenyl)-N- (3-(2-oxopiperidin-1- yl)propyl)benzo[d]imidazo [2,1-b]thiazole-7-carboxamide |
| 424 | 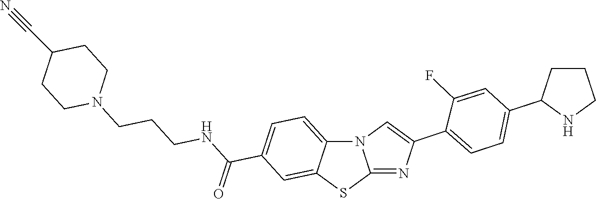 [SMILES: N#CC1CCN(CCCNC(=O)c2ccc3c(c2)sc2nc(-c4ccc(C5CCCN5)cc4F)cn23)CC1] | N-(3-(4-cyanopiperidin-1- yl)propyl)-2-(2-fluoro-4- (pyrrolidin-2- yl)phenyl)benzo[d]imidazo [2,1-b]thiazole-7-carboxamide |
| 425 | 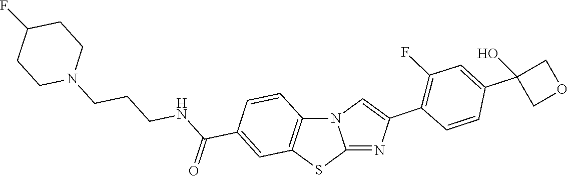 [SMILES: O=C(NCCCN1CCC(F)CC1)c1ccc2c(c1)sc1nc(-c3ccc(C4(O)COC4)cc3F)cn12] | 2-(2-fluoro-4-(3- hydroxyoxetan-3-yl)phenyl)- N-(3-(4-fluoropiperidin-1- yl)propyl)benzo[dlimidazo [2,1-b]thiazole-7-carboxamide |
| 426 | 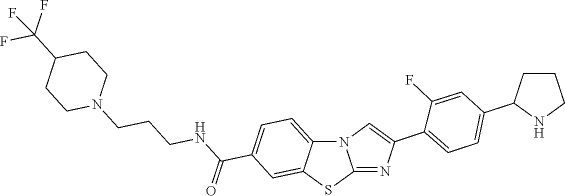 [SMILES: O=C(NCCCN1CCC(C(F)(F)F)CC1)c1ccc2c(c1)sc1nc(-c3ccc(C4CCCN4)cc3F)cn12] | 2-(2-fluoro-4-(pyrrolidin-2- yl)phenyl)-N-(3-(4- (trifluoromethyl)piperidin-1- yl)propyl)benzo[d]imidazo [2,1-b]thiazole-7-carboxamide |
| 427S | 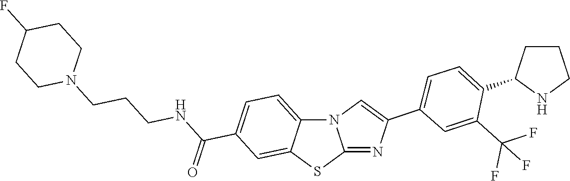 [SMILES: O=C(NCCCN1CCC(F)CC1)c1ccc2c(c1)sc1nc(-c3ccc([C@@H]4CCCN4)c(C(F)(F)F)c3)cn12] | (S)-N-(3-(4-fluoropiperidin- 1-yl)propyl)-2-(4- (pyrrolidin-2-yl)-3- (trifluoromethyl)phenyl)benzo [d]imidazo[2,1-b]thiazole- 7-carboxamide |
| 427R | 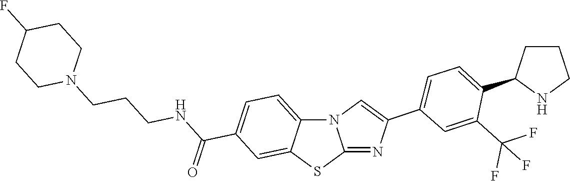 [SMILES: O=C(NCCCN1CCC(F)CC1)c1ccc2c(c1)sc1nc(-c3ccc([C@H]4CCCN4)c(C(F)(F)F)c3)cn12] | (R)-N-(3-(4-fluoropiperidin- 1-yl)propyl)-2-(4- (pyrrolidin-2-yl)-3- (trifluoromethyl)phenyl)benzo [d]imidazo[2,1-b]thiazole- 7-carboxamide |
| 428S | 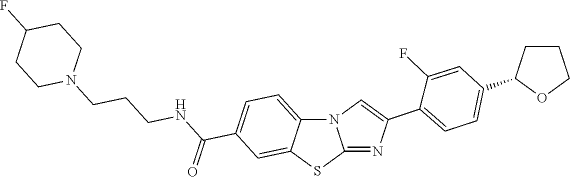 [SMILES: O=C(NCCCN1CCC(F)CC1)c1ccc2c(c1)sc1nc(-c3ccc([C@@H]4CCCO4)cc3F)cn12] | (S)-2-(2-fluoro-4- (tetrahydrofuran-2- yl)phenyl)-N-(3-(4- fluoropiperidin-1- yl)propyl)benzo[d]imidazo [2,1-b]thiazole-7-carboxamide |
| 428R | 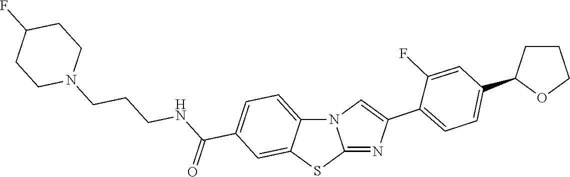 [SMILES: O=C(NCCCN1CCC(F)CC1)c1ccc2c(c1)sc1nc(-c3ccc([C@H]4CCCO4)cc3F)cn12] | (R)-2-(2-fluoro-4- (tetrahydrofuran-2- yl)phenyl)-N-(3-(4- fluoropiperidin-1- yl)propyl)benzo[d]imidazo [2,1-b]thiazole-7-carboxamide |
| 429S | 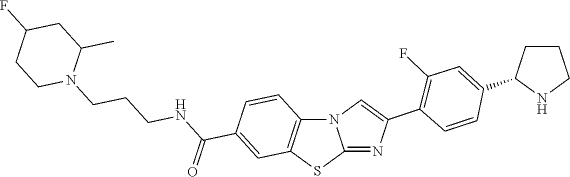 [SMILES: CC1CC(F)CCN1CCCNC(=O)c1ccc2c(c1)sc1nc(-c3ccc([C@@H]4CCCN4)cc3F)cn12] | N-(3-(4-fluoro-2- methylpiperidin-1-yl)propyl)- 2-(2-fluoro-4-((S)-pyrrolidin- 2- yl)phenyl)benzo[d]imidazo [2,1-b]thiazole-7-carboxamide |
| 429R | 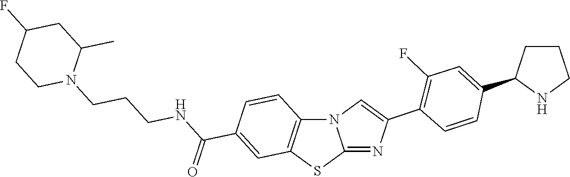 [SMILES: CC1CC(F)CCN1CCCNC(=O)c1ccc2c(c1)sc1nc(-c3ccc([C@H]4CCCN4)cc3F)cn12] | N-(3-(4-fluoro-2- methylpiperidin-1-yl)propyl)- 2-(2-fluoro-4-((R)- pyrrolidin-2- yl)phenyl)benzo[d]imidazo [2,1-b]thiazole-7-carboxamide |
| 430S | 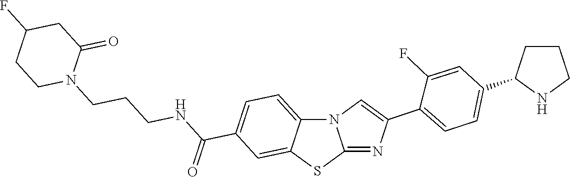 [SMILES: O=C(NCCCN1CCC(F)CC1=O)c1ccc2c(c1)sc1nc(-c3ccc([C@@H]4CCCN4)cc3F)cn12] | N-(3-(4-fluoro-2- oxopiperidin-1-yl)propyl)-2- (2-fluoro-4-((S)-pyrrolidin-2- yl)phenyl)benzo[d]imidazo [2,1-b]thiazole-7-carboxamide |
| 430R | 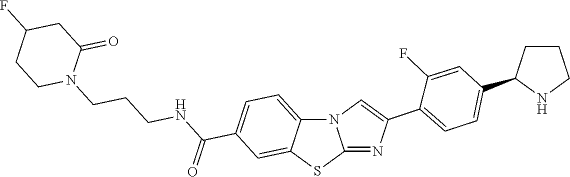 [SMILES: O=C(NCCCN1CCC(F)CC1=O)c1ccc2c(c1)sc1nc(-c3ccc([C@H]4CCCN4)cc3F)cn12] | N-(3-(4-fluoro-2- oxopiperidin-1-yl)propyl)-2- (2-fluoro-4-((R)-pyrrolidin- 2- yl)phenyl)benzo[d]imidazo [2,1-b]thiazole-7-carboxamide |
| 431S | 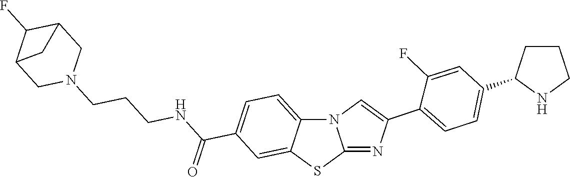 [SMILES: O=C(NCCCN1CC2CC(C1)C2F)c1ccc2c(c1)sc1nc(-c3ccc([C@@H]4CCCN4)cc3F)cn12] | N-(3-(6-fluoro-3- azabicyclo[3.1.1]heptan-3- yl)propyl)-2-(2-fluoro-4-((S)- pyrrolidin-2- yl)phenyl)benzo[d]imidazo [2,1-b]thiazole-7-carboxamide |
| 431R | 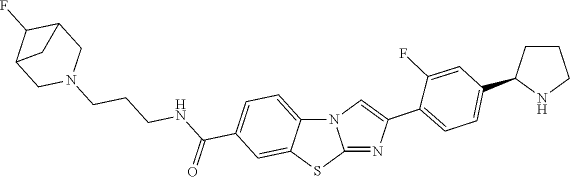 [SMILES: O=C(NCCCN1CC2CC(C1)C2F)c1ccc2c(c1)sc1nc(-c3ccc([C@H]4CCCN4)cc3F)cn12] | N-(3-(6-fluoro-3- azabicyclo[3.1.1]heptan-3- yl)propyl)-2-(2-fluoro-4- ((R)-pyrrolidin-2- yl)phenyl)benzo[d]imidazo [2,1-b]thiazole-7-carboxamide |
| 432S | 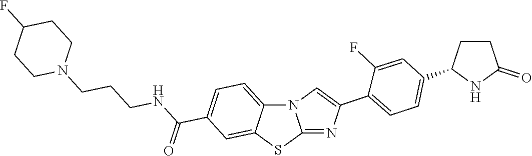 [SMILES: O=C1CC[C@@H](c2ccc(-c3cn4c(n3)sc3cc(C(=O)NCCCN5CCC(F)CC5)ccc34)c(F)c2)N1] | (S)-2-(2-fluoro-4-(5- oxopyrrolidin-2-yl)phenyl)- N-(3-(4-fluoropiperidin-1- yl)propyl)benzo[d]imidazo [2,1-b]thiazole-7-carboxamide |
| 432R | 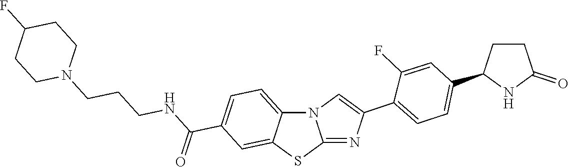 [SMILES: O=C1CC[C@H](c2ccc(-c3cn4c(n3)sc3cc(C(=O)NCCCN5CCC(F)CC5)ccc34)c(F)c2)N1] | (R)-2-(2-fluoro-4-(5- oxopyrrolidin-2-yl)phenyl)- N-(3-(4-fluoropiperidin-1- yl)propyl)benzo[d]imidazo [2-,1-b]thiazole-7- carboxamide |
| 433S | 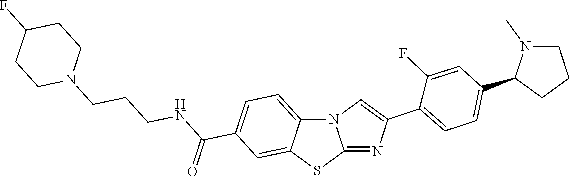 [SMILES: CN1CCC[C@H]1c1ccc(-c2cn3c(n2)sc2cc(C(=O)NCCCN4CCC(F)CC4)ccc23)c(F)c1] | (S)-2-(2-fluoro-4-(1- methylpyrrolidin-2- yl)phenyl)-N-(3-(4- fluoropiperidin-1- yl)propyl)benzo[d]imidazo [2,1-b]thiazole-7- carboxamide |
| 433R | 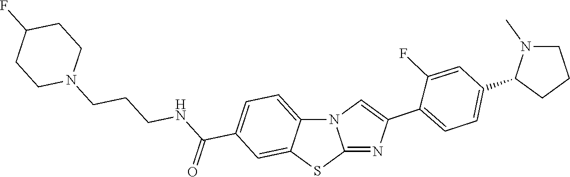 [SMILES: CN1CCC[C@@H]1c1ccc(-c2cn3c(n2)sc2cc(C(=O)NCCCN4CCC(F)CC4)ccc23)c(F)c1] | (R)-2-(2-fluoro-4-(1- methylpyrrolidin-2- yl)phenyl)-N-(3-(4- fluoropiperidin-1- yl)propyl)benzo[d]imidazo [2,1-b]thiazole-7- carboxamide |
| 434S | 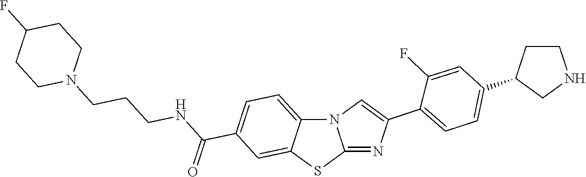 [SMILES: O=C(NCCCN1CCC(F)CC1)c1ccc2c(c1)sc1nc(-c3ccc([C@@H]4CCNC4)cc3F)cn12] | (S)-2-(2-fluoro-4-(pyrrolidin- 3-yl)phenyl)-N-(3-(4- fluoropiperidin-1- yl)propyl)benzo[d]imidazo [2,1-b]thiazole-7-carboxamide |
| 434R | 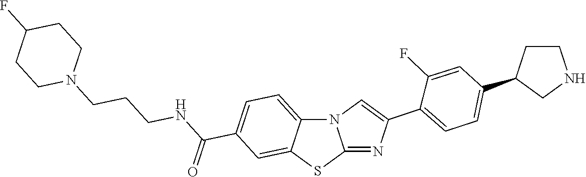 [SMILES: O=C(NCCCN1CCC(F)CC1)c1ccc2c(c1)sc1nc(-c3ccc([C@H]4CCNC4)cc3F)cn12] | (R)-2-(2-fluoro-4-(pyrrolidin- 3-yl)phenyl)-N-(3-(4- fluoropiperidin-1- yl)propyl)benzo[d]imidazo [2,1-b]thiazole-7-carboxamide |
| 435S | 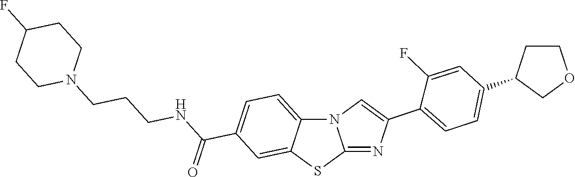 [SMILES: O=C(NCCCN1CCC(F)CC1)c1ccc2c(c1)sc1nc(-c3ccc([C@@H]4CCOC4)cc3F)cn12] | (S)-2-(2-fluoro-4- (tetrahydrofuran-3- yl)phenyl)-N-(3-(4- fluoropiperidin-1- yl)propyl)benzo[d]imidazo [2,1-b]thiazole-7-carboxamide |
| 435R | 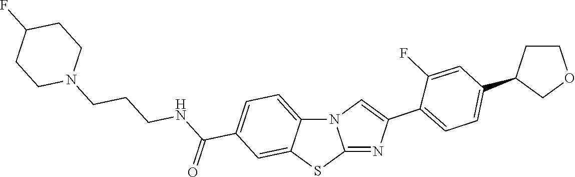 [SMILES: O=C(NCCCN1CCC(F)CC1)c1ccc2c(c1)sc1nc(-c3ccc([C@H]4CCOC4)cc3F)cn12] | (R)-2-(2-fluoro-4- (tetrahydrofuran-3- yl)phenyl)-N-(3-(4- fluoropiperidin-1- yl)propyl)benzo[d]imidazo [2,1-b]thiazole-7-carboxamide |
| 436S | 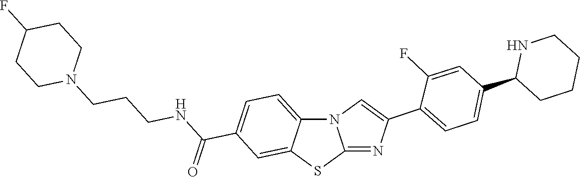 [SMILES: O=C(NCCCN1CCC(F)CC1)c1ccc2c(c1)sc1nc(-c3ccc([C@@H]4CCCCN4)cc3F)cn12] | (S)-2-(2-fluoro-4-(piperidin- 2-yl)phenyl)-N-(3-(4- fluoropiperidin-1- yl)propyl)benzo[d]imidazo [2,1-b]thiazole-7-carboxamide |
| 436R | 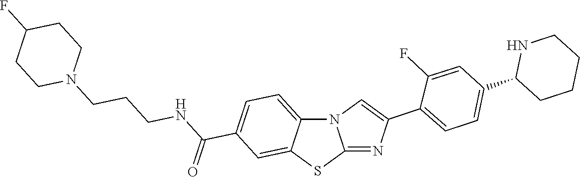 [SMILES: O=C(NCCCN1CCC(F)CC1)c1ccc2c(c1)sc1nc(-c3ccc([C@H]4CCCCN4)cc3F)cn12] | (R)-2-(2-fluoro-4-(piperidin- 2-yl)phenyl)-N-(3-(4- fluoropiperidin-1- yl)propyl)benzo[d]imidazo [2,1-b]thiazole-7-carboxamide |
| 437S | 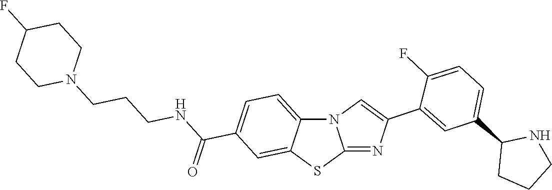 [SMILES: O=C(NCCCN1CCC(F)CC1)c1ccc2c(c1)sc1nc(-c3cc([C@@H]4CCCN4)ccc3F)cn12] | (S)-2-(2-fluoro-5-(pyrrolidin- 2-yl)phenyl)-N-(3-(4- fluoropiperidin-1- yl)propyl)benzo[d]imidazo [2,1-b]thiazole-7-carboxamide |
| 437R | 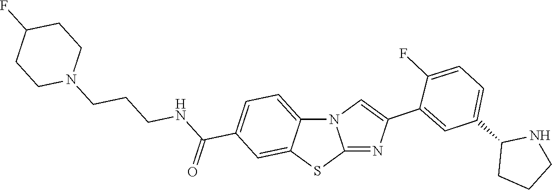 [SMILES: O=C(NCCCN1CCC(F)CC1)c1ccc2c(c1)sc1nc(-c3cc([C@H]4CCCN4)ccc3F)cn12] | (R)-2-(2-fluoro-5- (pyrrolidin-2-yl)phenyl)-N- (3-(4-fluoropiperidin-1- yl)propyl)benzo[d]imidazo [2,1-b]thiazole-7-carboxamide |
| 438S | 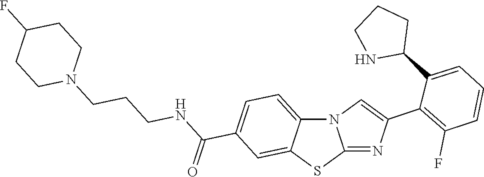 [SMILES: O=C(NCCCN1CCC(F)CC1)c1ccc2c(c1)sc1nc(-c3c(F)cccc3[C@@H]3CCCN3)cn12] | (S)-2-(2-fluoro-6-(pyrrolidin- 2-yl)phenyl)-N-(3-(4- fluoropiperidin-1- yl)propyl)benzo[d]imidazo [2,1-b]thiazole-7-carboxamide |
| 438R | 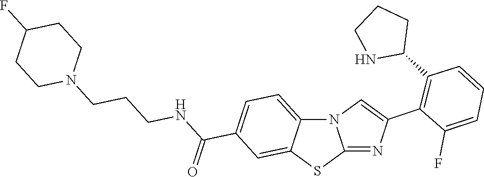 [SMILES: O=C(NCCCN1CCC(F)CC1)c1ccc2c(c1)sc1nc(-c3c(F)cccc3[C@H]3CCCN3)cn12] | (R)-2-(2-fluoro-6- (pyrrolidin-2-yl)phenyl)-N- (3-(4-fluoropiperidin-1- yl)propyl)benzo[d]imidazo [2,1-b]thiazole-7-carboxamide |
| 439S | 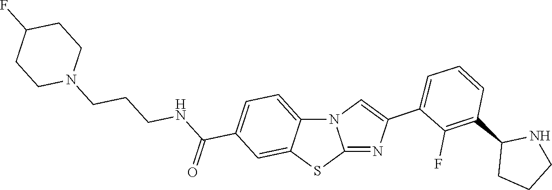 [SMILES: O=C(NCCCN1CCC(F)CC1)c1ccc2c(c1)sc1nc(-c3cccc([C@@H]4CCCN4)c3F)cn12] | (S)-2-(2-fluoro-3-(pyrrolidin- 2-yl)phenyl)-N-(3-(4- fluoropiperidin-1-yl)propyl) benzo[d]imidazo[2,1- b]thiazole-7-carboxamide |
| 439R | 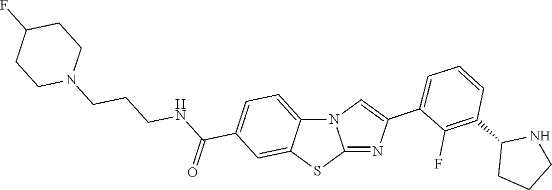 [SMILES: O=C(NCCCN1CCC(F)CC1)c1ccc2c(c1)sc1nc(-c3cccc([C@H]4CCCN4)c3F)cn12] | (R)-2-(2-fluoro-3- (pyrrolidin-2-yl)phenyl)-N- (3-(4-fluoropiperidin-1- yl)propyl)benzo[d]imidazo [2,1-b]thiazole-7-carboxamide |
| 440 | 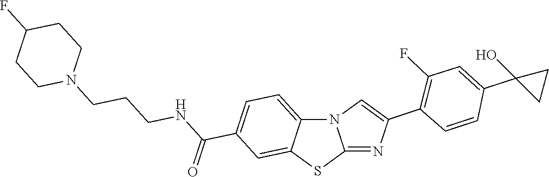 [SMILES: O=C(NCCCN1CCC(F)CC1)c1ccc2c(c1)sc1nc(-c3ccc(C4(O)CC4)cc3F)cn12] | 2-(2-fluoro-4-(1- hydroxycyclopropyl)phenyl)- N-(3-(4-fluoropiperidin-1- yl)propyl)benzo[d]imidazo [2,1-b]thiazole-7-carboxamide |
| 441 | 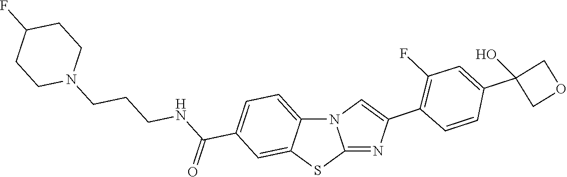 [SMILES: O=C(NCCCN1CCC(F)CC1)c1ccc2c(c1)sc1nc(-c3ccc(C4(O)COC4)cc3F)cn12] | 2-(2-fluoro-4-(3- hydroxyoxetan-3-yl)phenyl)- N-(3-(4-fluoropiperidin-1- yl)propyl)benzo[d]imidazo [2,1-b]thiazole-7-carboxamide |
| 442 | 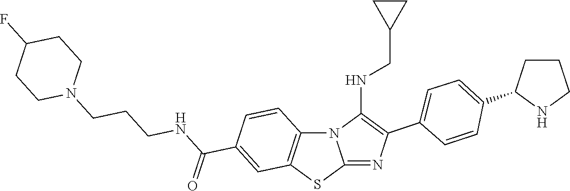 [SMILES: O=C(NCCCN1CCC(F)CC1)c1ccc2c(c1)sc1nc(-c3ccc([C@@H]4CCCN4)cc3)c(NCC3CC3)n12] | (S)-3- ((cyclopropylmethyl)amino)- N-(3-(4-fluoropiperidin-1- yl)propyl)-2-(4-(pyrrolidin- 2- yl)phenyl)benzo[d]imidazo [2,1-b]thiazole-7-carboxamide |
| 443 | 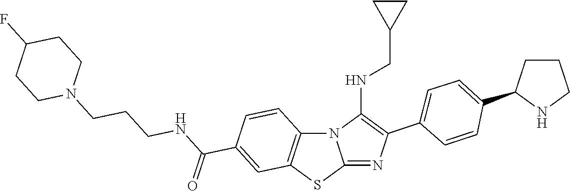 [SMILES: O=C(NCCCN1CCC(F)CC1)c1ccc2c(c1)sc1nc(-c3ccc([C@H]4CCCN4)cc3)c(NCC3CC3)n12] | (R)-3- ((cyclopropylmethyl)amino)- N-(3-(4-fluoropiperidin-1- yl)propyl)-2-(4-(pyrrolidin- 2- yl)phenyl)benzo[d]imidazo [2,1-b]thiazole-7-carboxamide |
| 444 | 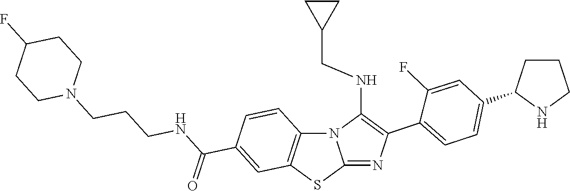 [SMILES: O=C(NCCCN1CCC(F)CC1)c1ccc2c(c1)sc1nc(-c3ccc([C@@H]4CCCN4)cc3F)c(NCC3CC3)n12] | (S)-3- ((cyclopropylmethyl)amino)- 2-(2-fluoro-4-(pyrrolidin-2- yl)phenyl)-N-(3-(4- fluoropiperidin-1- yl)propyl)benzo[d]imidazo [2,1-b]thiazole-7-carboxamide |
| 445 | 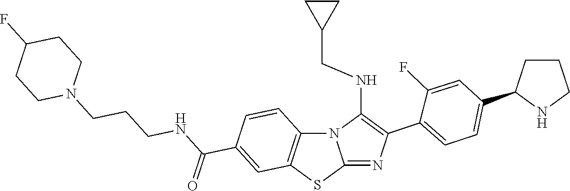 [SMILES: O=C(NCCCN1CCC(F)CC1)c1ccc2c(c1)sc1nc(-c3ccc([C@H]4CCCN4)cc3F)c(NCC3CC3)n12] | (R)-3- ((cyclopropylmethyl)amino)- 2-(2-fluoro-4-(pyrrolidin-2- yl)phenyl)-N-(3-(4- fluoropiperidin-1- yl)propyl)benzo[d]imidazo [2,1-b]thiazole-7-carboxamide |
| 446 | 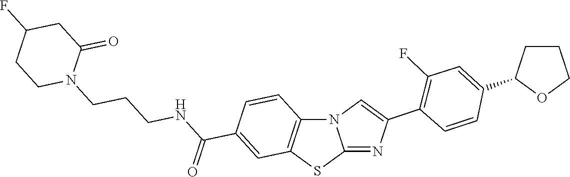 [SMILES: O=C(NCCCN1CCC(F)CC1=O)c1ccc2c(c1)sc1nc(-c3ccc([C@@H]4CCCO4)cc3F)cn12] | N-(3-(4-fluoro-2- oxopiperidin-1-yl)propyl)-2- (2-fluoro-4-((S)- tetrahydrofuran-2- yl)phenyl)benzo[d]imidazo [2,1-b]thiazole-7-carboxamide |
| 447 | 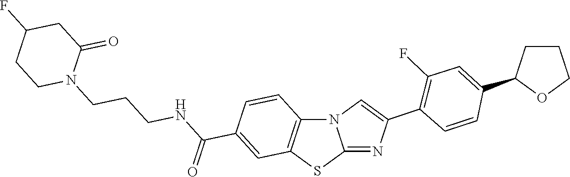 [SMILES: O=C(NCCCN1CCC(F)CC1=O)c1ccc2c(c1)sc1nc(-c3ccc([C@H]4CCCO4)cc3F)cn12] | N-(3-(4-fluoro-2- oxopiperidin-1-yl)propyl)-2- (2-fluoro-4-((S)- tetrahydrofuran-2- yl)phenyl)benzo[d]imidazo [2,1-b]thiazole-7-carboxamide |
| 448 | 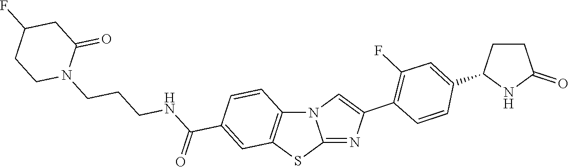 [SMILES: O=C1CC[C@@H](c2ccc(-c3cn4c(n3)sc3cc(C(=O)NCCCN5CCC(F)CC5=O)ccc34)c(F)c2)N1] | N-(3-(4-fluoro-2- oxopiperidin-1-yl)propyl)-2- (2-fluoro-4-((S)-5- oxopyrrolidin-2- yl)phenyl)benzo[d]imidazo [2,1-b]thiazole-7-carboxamide |
| 449 | 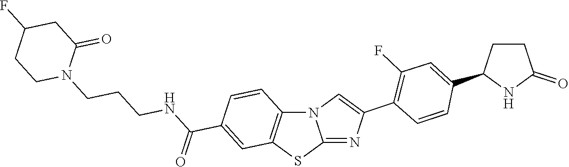 [SMILES: O=C1CC[C@H](c2ccc(-c3cn4c(n3)sc3cc(C(=O)NCCCN5CCC(F)CC5=O)ccc34)c(F)c2)N1] | N-(3-(4-fluoro-2- oxopiperidin-1-yl)propyl)-2- (2-fluoro-4-((R)-5- oxopyrrolidin-2- yl)phenyl)benzo[d]imidazo [2,1-b]thiazole-7-carboxamide |
| 450S | 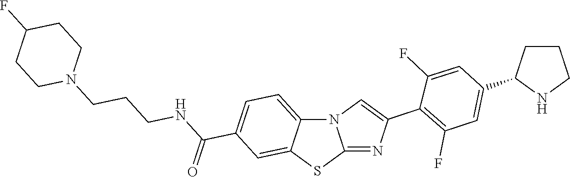 [SMILES: O=C(NCCCN1CCC(F)CC1)c1ccc2c(c1)sc1nc(-c3c(F)cc([C@@H]4CCCN4)cc3F)cn12] | (S)-2-(2,6-difluoro-4- (pyrrolidin-2-yl)phenyl)-N- (3-(4-fluoropiperidin-1- yl)propyl)benzo[d]imidazo [2,1-b]thiazole-7-carboxamide |
| 450R | 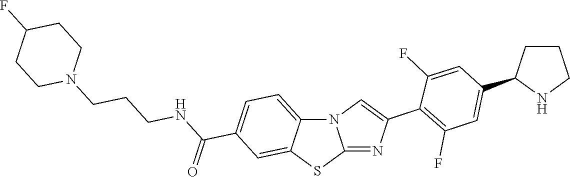 [SMILES: O=C(NCCCN1CCC(F)CC1)c1ccc2c(c1)sc1nc(-c3c(F)cc([C@H]4CCCN4)cc3F)cn12] | (R)-2-(2,6-difluoro-4- (pyrrolidin-2-yl)phenyl)-N- (3-(4-fluoropiperidin-1- yl)propyl)benzo[d]imidazo [2,1-b]thiazole-7-carboxamide |
| 451S | 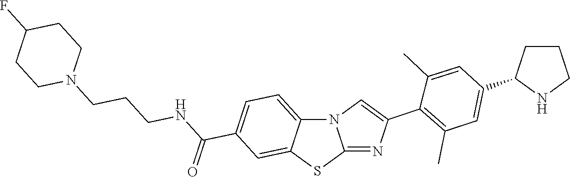 [SMILES: Cc1cc([C@@H]2CCCN2)cc(C)c1-c1cn2c(n1)sc1cc(C(=O)NCCCN3CCC(F)CC3)ccc12] | (S)-2-(2,6-dimethyl-4- (pyrrolidin-2-yl)phenyl)-N- (3-(4-fluoropiperidin-1- yl)propyl)benzo[d]imidazo [2,1-b]thiazole-7-carboxamide |
| 451R | 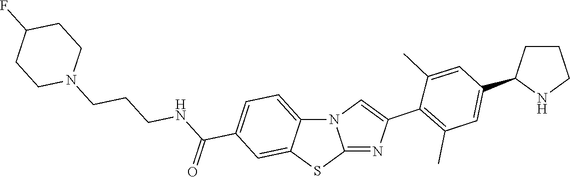 [SMILES: Cc1cc([C@H]2CCCN2)cc(C)c1-c1cn2c(n1)sc1cc(C(=O)NCCCN3CCC(F)CC3)ccc12] | (R)-2-(2,6-dimethyl-4- (pyrrolidin-2-yl)phenyl)-N- (3-(4-fluoropiperidin-1- yl)propyl)benzo[d]imidazo [2,1-b]thiazole-7-carboxamide |
| 452S | 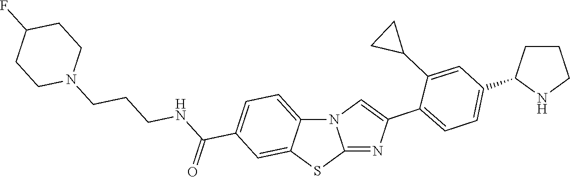 [SMILES: O=C(NCCCN1CCC(F)CC1)c1ccc2c(c1)sc1nc(-c3ccc([C@@H]4CCCN4)cc3C3CC3)cn12] | (S)-2-(2-cyclopropyl-4- (pyrrolidin-2-yl)phenyl)-N- (3-(4-fluoropiperidin-1- yl)propyl)benzo[d]imidazo [2,1-b]thiazole-7-carboxamide |
| 452R | 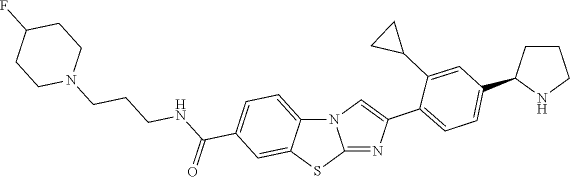 [SMILES: O=C(NCCCN1CCC(F)CC1)c1ccc2c(c1)sc1nc(-c3ccc([C@H]4CCCN4)cc3C3CC3)cn12] | (R)-2-(2-cyclopropyl-4- (pyrrolidin-2-yl)phenyl)-N- (3-(4-fluoropiperidin-1- yl)propyl)benzo[d]imidazo [2,1-b]thiazole-7-carboxamide |
| 453S | 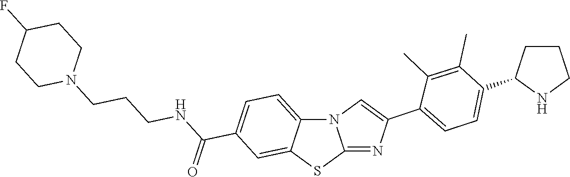 [SMILES: Cc1c(-c2cn3c(n2)sc2cc(C(=O)NCCCN4CCC(F)CC4)ccc23)ccc([C@@H]2CCCN2)c1C] | (S)-2-(2,3-dimethyl-4- (pyrrolidin-2-yl)phenyl)-N- (3-(4-fluoropiperidin-1- yl)propyl)benzo[d]imidazo [2,1-b]thiazole-7-carboxamide |
| 453R | 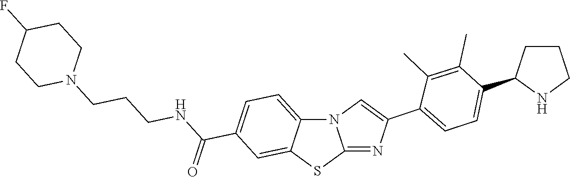 [SMILES: Cc1c(-c2cn3c(n2)sc2cc(C(=O)NCCCN4CCC(F)CC4)ccc23)ccc([C@H]2CCCN2)c1C] | (R)-2-(2,3-dimethyl-4- (pyrrolidin-2-yl)phenyl)-N- (3-(4-fluoropiperidin-1- yl)propyl)benzo[d]imidazo [2,1-b]thiazole-7-carboxamide |
| 454 | 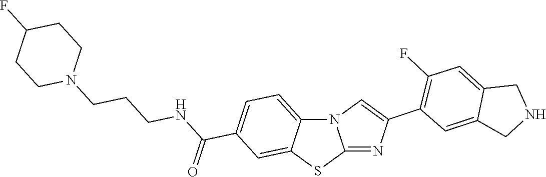 [SMILES: O=C(NCCCN1CCC(F)CC1)c1ccc2c(c1)sc1nc(-c3cc4c(cc3F)CNC4)cn12] | 2-(6-fluoroisoindolin-5-yl)- N-(3-(4-fluoropiperidin-1- yl)propyl)benzo[d]imidazo [2,1-b]thiazole-7-carboxamide |
| 455 | 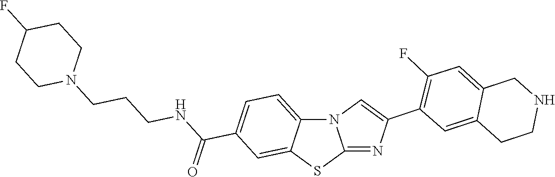 [SMILES: O=C(NCCCN1CCC(F)CC1)c1ccc2c(c1)sc1nc(-c3cc4c(cc3F)CNCC4)cn12] | 2-(7-fluoro-1,2,3,4- tetrahydroisoquinolin-6-yl)- N-(3-(4-fluoropiperidin-1- yl)propyl)benzo[d]imidazo [2,1-b]thiazole-7-carboxamide |
| 456 | 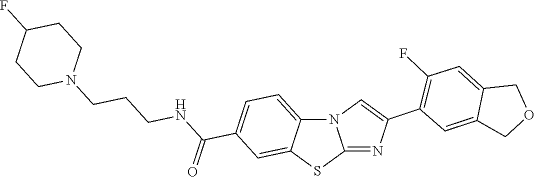 [SMILES: O=C(NCCCN1CCC(F)CC1)c1ccc2c(c1)sc1nc(-c3cc4c(cc3F)COC4)cn12] | 2-(6-fluoro-1,3- dihydroisobenzofuran-5-yl)- N-(3-(4-fluoropiperidin-1- yl)propyl)benzo[d]imidazo [2,1-b]thiazole-7-carboxamide |
| 457 | 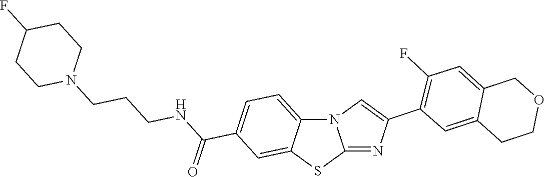 [SMILES: O=C(NCCCN1CCC(F)CC1)c1ccc2c(c1)sc1nc(-c3cc4c(cc3F)COCC4)cn12] | 2-(7-fluoroisochroman-6-yl)- N-(3-(4-fluoropiperidin-1- yl)propyl)benzo[d]imidazo [2,1-b]thiazole-7-carboxamide |
| 458 | 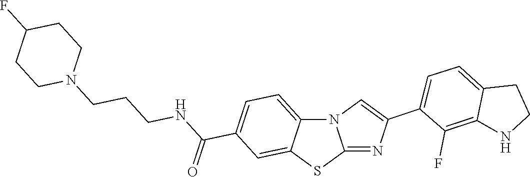 [SMILES: O=C(NCCCN1CCC(F)CC1)c1ccc2c(c1)sc1nc(-c3ccc4c(c3F)NCC4)cn12] | 2-(7-fluoroindolin-6-yl)-N- (3-(4-fluoropiperidin-1- yl)propyl)benzo[d]imidazo [2,1-b]thiazole-7-carboxamide |
| 459 | 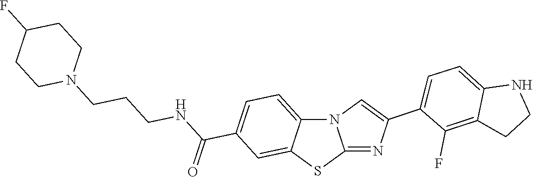 [SMILES: O=C(NCCCN1CCC(F)CC1)c1ccc2c(c1)sc1nc(-c3ccc4c(c3F)CCN4)cn12] | 2-(4-fluoroindolin-5-yl)-N- (3-(4-fluoropiperidin-1- yl)propyl)benzo[d]imidazo [2,1-b]thiazole-7-carboxamide |
| 460S | 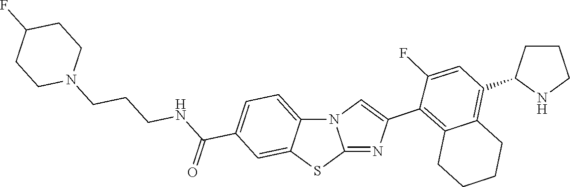 [SMILES: O=C(NCCCN1CCC(F)CC1)c1ccc2c(c1)sc1nc(-c3c(F)cc([C@@H]4CCCN4)c4c3CCCC4)cn12] | (S)-2-(2-fluoro-4-(pyrrolidin- 2-yl)-5,6,7,8- tetrahydronaphthalen-1-yl)- N-(3-(4-fluoropiperidin-1- yl)propyl)benzo[d]imidazo [2,1-b]thiazole-7-carboxamide |
| 460R | 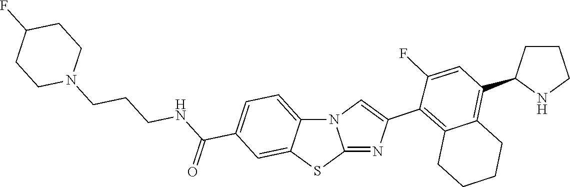 [SMILES: O=C(NCCCN1CCC(F)CC1)c1ccc2c(c1)sc1nc(-c3c(F)cc([C@H]4CCCN4)c4c3CCCC4)cn12] | (R)-2-(2-fluoro-4- (pyrrolidin-2-yl)-5,6,7,8- tetrahydronaphthalen-1-yl)- N-(3-(4-fluoropiperidin-1- yl)propyl)benzo[d]imidazo [2,1-b]thiazole-7-carboxamide |
| 461S | 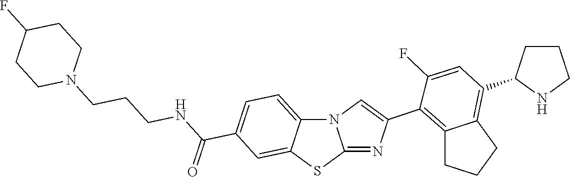 [SMILES: O=C(NCCCN1CCC(F)CC1)c1ccc2c(c1)sc1nc(-c3c(F)cc([C@@H]4CCCN4)c4c3CCC4)cn12] | (S)-2-(5-fluoro-7-(pyrrolidin- 2-yl)-2,3-dihydro-1H-inden- 4-yl)-N-(3-(4- fluoropiperidin-1- yl)propyl)benzo[d]imidazo [2,1-b]thiazole-7-carboxamide |
| 461R | 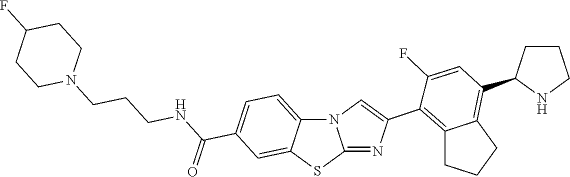 [SMILES: O=C(NCCCN1CCC(F)CC1)c1ccc2c(c1)sc1nc(-c3c(F)cc([C@H]4CCCN4)c4c3CCC4)cn12] | (R)-2-(5-fluoro-7- (pyrrolidin-2-yl)-2,3- dihydro-1H-inden-4-yl)-N- (3-(4-fluoropiperidin-1- yl)propyl)benzo[d]imidazo [2,1-b]thiazole-7-carboxamide |
| 462S | 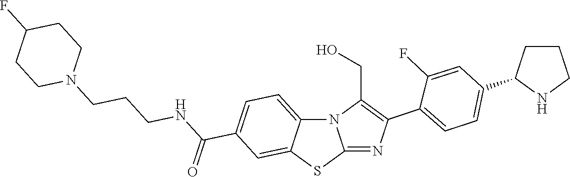 [SMILES: O=C(NCCCN1CCC(F)CC1)c1ccc2c(c1)sc1nc(-c3ccc([C@@H]4CCCN4)cc3F)c(CO)n12] | (S)-2-(2-fluoro-4-(pyrrolidin- 2-yl)phenyl)-N-(3-(4- fluoropiperidin-1-yl)propyl)- 3- (hydroxymethyl)benzo[d] imidazo[2,1-b]thiazole-7- carboxamide |
| 462R | 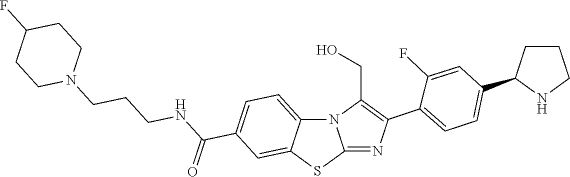 [SMILES: O=C(NCCCN1CCC(F)CC1)c1ccc2c(c1)sc1nc(-c3ccc([C@H]4CCCN4)cc3F)c(CO)n12] | (R)-2-(2-fluoro-4- (pyrrolidin-2-yl)phenyl)-N- (3-(4-fluoropiperidin-1- yl)propyl)-3- (hydroxymethyl)benzo[d] imidazo[2,1-b]thiazole-7- carboxamide |
| 463S | 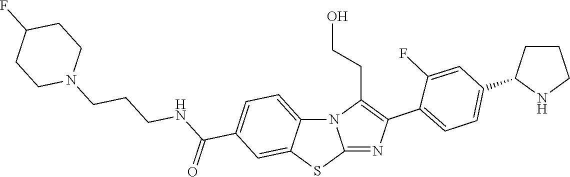 [SMILES: O=C(NCCCN1CCC(F)CC1)c1ccc2c(c1)sc1nc(-c3ccc([C@@H]4CCCN4)cc3F)c(CCO)n12] | (S)-2-(2-fluoro-4-(pyrrolidin- 2-yl)phenyl)-N-(3-(4- fluoropiperidin-1-yl)propyl)- 3-(2- hydroxyethyl)benzo[d] imidazo[2,1-b]thiazole- 7-carboxamide |
| 463R | 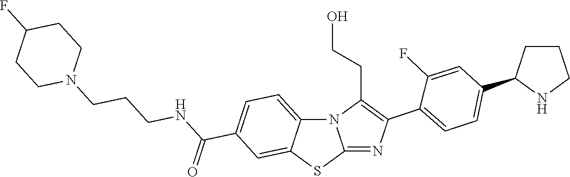 [SMILES: O=C(NCCCN1CCC(F)CC1)c1ccc2c(c1)sc1nc(-c3ccc([C@H]4CCCN4)cc3F)c(CCO)n12] | (R)-2-(2-fluoro-4- (pyrrolidin-2-yl)phenyl)-N- (3-(4-fluoropiperidin-1- yl)propyl)-3-(2- hydroxyethyl)benzo[d] imidazo[2,1-b]thiazole- 7-carboxamide |
| 464S | 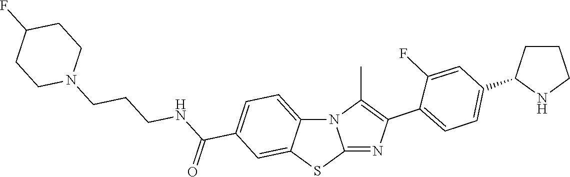 [SMILES: Cc1c(-c2ccc([C@@H]3CCCN3)cc2F)nc2sc3cc(C(=O)NCCCN4CCC(F)CC4)ccc3n12] | (S)-2-(2-fluoro-4-(pyrrolidin- 2-yl)phenyl)-N-(3-(4- fluoropiperidin-1-yl)propyl)- 3- methylbenzo[d]imidazo [2,1-b]thiazole-7-carboxamide |
| 464R | 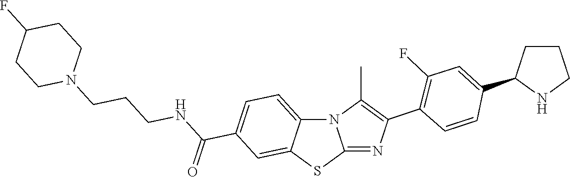 [SMILES: Cc1c(-c2ccc([C@H]3CCCN3)cc2F)nc2sc3cc(C(=O)NCCCN4CCC(F)CC4)ccc3n12] | (R)-2-(2-fluoro-4- (pyrrolidin-2-yl)phenyl)-N- (3-(4-fluoropiperidin-1- yl)propyl)-3- methylbenzo[d]imidazo [2,1-b]thiazole-7-carboxamide |
| 465 | 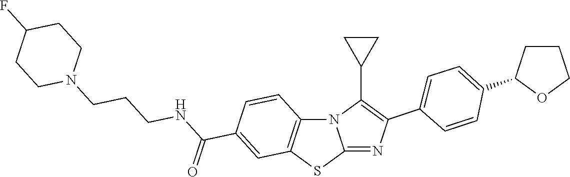 [SMILES: O=C(NCCCN1CCC(F)CC1)c1ccc2c(c1)sc1nc(-c3ccc([C@@H]4CCCO4)cc3)c(C3CC3)n12] | (S)-3-cyclopropyl-N-(3-(4- fluoropiperidin-1-yl)propyl)- 2-(4-(tetrahydrofuran-2- yl)phenyl)benzo[d]imidazo [2,1-b]thiazole-7-carboxamide |
| 466 | 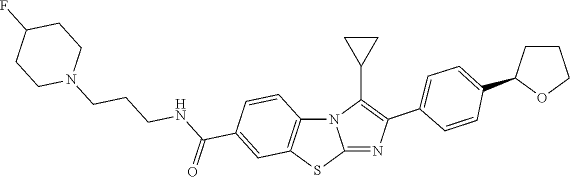 [SMILES: O=C(NCCCN1CCC(F)CC1)c1ccc2c(c1)sc1nc(-c3ccc([C@H]4CCCO4)cc3)c(C3CC3)n12] | (R)-3-cyclopropyl-N-(3-(4- fluoropiperidin-1-yl)propyl)- 2-(4-(tetrahydrofuran-2- yl)phenyl)benzo[d]imidazo [2,1-b]thiazole-7-carboxamide |
| 467 | 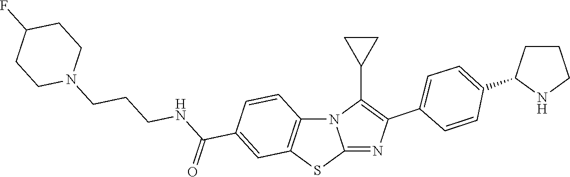 [SMILES: O=C(NCCCN1CCC(F)CC1)c1ccc2c(c1)sc1nc(-c3ccc([C@@H]4CCCN4)cc3)c(C3CC3)n12] | (S)-3-cyclopropyl-N-(3-(4- fluoropiperidin-1-yl)propyl)- 2-(4-(pyrrolidin-2- yl)phenyl)benzo[d]imidazo [2,1-b]thiazole-7-carboxamide |
| 468 | 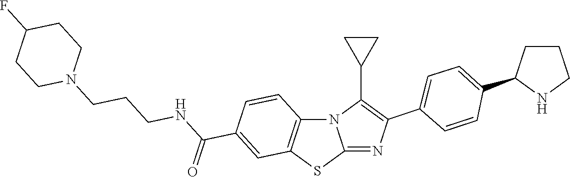 [SMILES: O=C(NCCCN1CCC(F)CC1)c1ccc2c(c1)sc1nc(-c3ccc([C@H]4CCCN4)cc3)c(C3CC3)n12] | (R)-3-cyclopropyl-N-(3-(4- fluoropiperidin-1-yl)propyl)- 2-(4-(pyrrolidin-2- yl)phenyl)benzo[d]imidazo [2,1-b]thiazole-7-carboxamide |
| 469S | 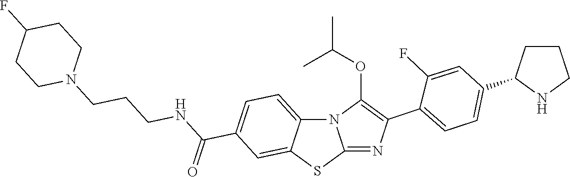 [SMILES: CC(C)Oc1c(-c2ccc([C@@H]3CCCN3)cc2F)nc2sc3cc(C(=O)NCCCN4CCC(F)CC4)ccc3n12] | (S)-2-(2-fluoro-4-(pyrrolidin- 2-yl)phenyl)-N-(3-(4- fluoropiperidin-1-yl)propyl)- 3- isopropoxybenzo[d]imidazo [2,1-b]thiazole-7- carboxamide |
| 469R | 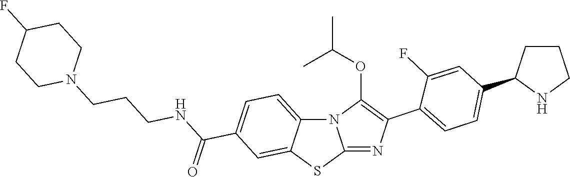 [SMILES: CC(C)Oc1c(-c2ccc([C@H]3CCCN3)cc2F)nc2sc3cc(C(=O)NCCCN4CCC(F)CC4)ccc3n12] | (R)-2-(2-fluoro-4- (pyrrolidin-2-yl)phenyl)-N- (3-(4-fluoropiperidin-1- yl)propyl)-3- isopropoxybenzo[d]imidazo [2,1-b]thiazole-7- carboxamide |
| 470S | 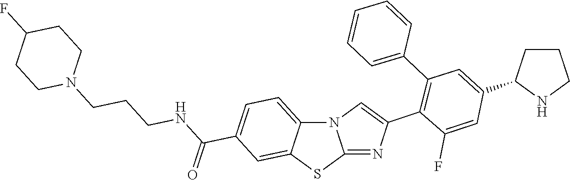 [SMILES: O=C(NCCCN1CCC(F)CC1)c1ccc2c(c1)sc1nc(-c3c(F)cc([C@@H]4CCCN4)cc3-c3ccccc3)cn12] | (S)-2-(3-fluoro-5-(pyrrolidin- 2-yl)-[1,1′-biphenyl]-2-yl)-N- (3-(4-fluoropiperidin-1- yl)propyl)benzo[d]imidazo [2,1-b]thiazole-7-carboxamide |
| 470R | 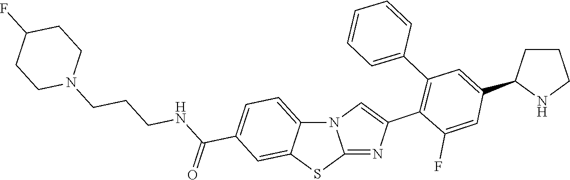 [SMILES: O=C(NCCCN1CCC(F)CC1)c1ccc2c(c1)sc1nc(-c3c(F)cc([C@H]4CCCN4)cc3-c3ccccc3)cn12] | (R)-2-(3-fluoro-5-(pyrrolidin- 2-yl)-[1,1′-biphenyl]-2-yl)-N- (3-(4-fluoropiperidin-1- yl)propyl)benzo[d]imidazo [2,1-b]thiazole-7-carboxamide |
| 471S | 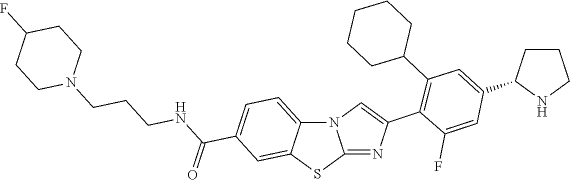 [SMILES: O=C(NCCCN1CCC(F)CC1)c1ccc2c(c1)sc1nc(-c3c(F)cc([C@@H]4CCCN4)cc3C3CCCCC3)cn12] | (S)-2-(2-cyclohexyl-6-fluoro- 4-(pyrrolidin-2-yl)phenyl)-N- (3-(4-fluoropiperidin-1- yl)propyl)benzo[d]imidazo [2,1-b]thiazole-7-carboxamide |
| 471R | 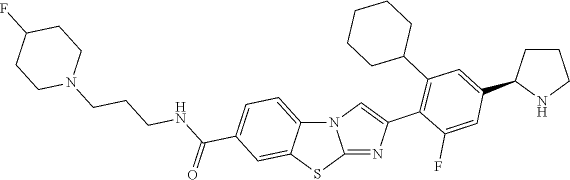 [SMILES: O=C(NCCCN1CCC(F)CC1)c1ccc2c(c1)sc1nc(-c3c(F)cc([C@H]4CCCN4)cc3C3CCCCC3)cn12] | (R)-2-(2-cyclohexyl-6-fluoro- 4-(pyrrolidin-2-yl)phenyl)-N- (3-(4-fluoropiperidin-1- yl)propyl)benzo[d]imidazo [2,1-b]thiazole-7-carboxamide |
| 472S | 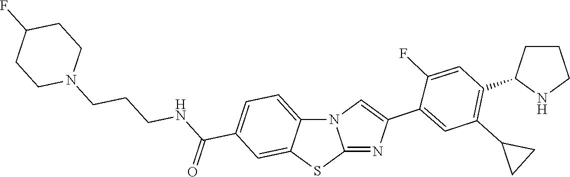 [SMILES: O=C(NCCCN1CCC(F)CC1)c1ccc2c(c1)sc1nc(-c3cc(C4CC4)c([C@@H]4CCCN4)cc3F)cn12] | (S)-2-(5-cyclopropyl-2- fluoro-4-(pyrrolidin-2- yl)phenyl)-N-(3-(4- fluoropiperidin-1- yl)propyl)benzo[d]imidazo [2,1-b]thiazole-7-carboxamide |
| 472R | 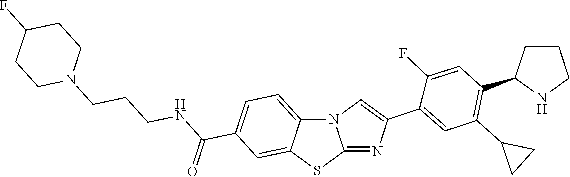 [SMILES: O=C(NCCCN1CCC(F)CC1)c1ccc2c(c1)sc1nc(-c3cc(C4CC4)c([C@H]4CCCN4)cc3F)cn12] | (R)-2-(5-cyclopropyl-2- fluoro-4-(pyrrolidin-2- yl)phenyl)-N-(3-(4- fluoropiperidin-1- yl)propyl)benzo[d]imidazo [2,1-b]thiazole-7-carboxamide |
| 473S | 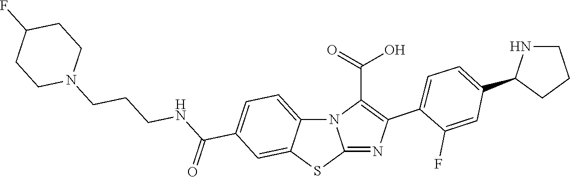 [SMILES: O=C(NCCCN1CCC(F)CC1)c1ccc2c(c1)sc1nc(-c3ccc([C@@H]4CCCN4)cc3F)c(C(=O)O)n12] | (S)-2-(2-fluoro-4-(pyrrolidin- 2-yl)phenyl)-7-((3-(4- fluoropiperidin-1- yl)propyl)carbamoyl)benzo [d]imidazo[2,1-b]thiazole-3- carboxylic acid |
| 473R | 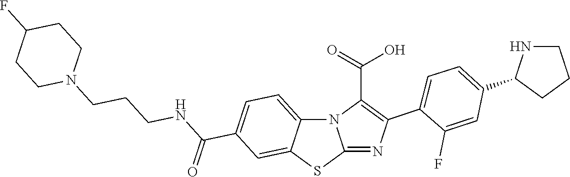 [SMILES: O=C(NCCCN1CCC(F)CC1)c1ccc2c(c1)sc1nc(-c3ccc([C@H]4CCCN4)cc3F)c(C(=O)O)n12] | (R)-2-(2-fluoro-4- (pyrrolidin-2-yl)phenyl)-7- ((3-(4-fluoropiperidin-1- yl)propyl)carbamoyl)benzo [d]imidazo[2,1-b]thiazole-3- carboxylic acid |

It is well understood that in structures presented in this invention wherein the carbon atom has less than 4 bonds, H atoms are present to complete the valence of the carbon. It is well understood that in structures presented in this invention wherein the nitrogen atom has less than 3 bonds, H atoms are present to complete the valence of the nitrogen.

In some embodiments, this invention is directed to the compounds listed hereinabove, pharmaceutical compositions and/or method of use thereof, wherein the compound is pharmaceutically acceptable salt, stereoisomer, tautomer, hydrate, N-oxide, reverse amide analog, prodrug, isotopic variant (deuterated analog), PROTAC, pharmaceutical product or any combination thereof. In some embodiments, the compounds are c-MYC mRNA translation modulators. In some embodiments, the compounds are c-MYC mRNA translation inhibitors. In some embodiments, the compounds are c-MYC inhibitors. In various embodiments, the compounds are a c-MYC mRNA transcription regulators. In various embodiments, the compounds are any combination of c-MYC mLRNA transcription regulators, c-MYC mRNA transcription regulators and c-MYC inhibitors.

As used herein, the term “alkyl” can be any straight- or branched-chain alkyl group containing up to about 30 carbons unless otherwise specified. In various embodiments, an alkyl includes C₁-C₅ carbons. In some embodiments, an alkyl includes C₁-C₆ carbons. In some embodiments, an alkyl includes C₁-C₅ carbons. In some embodiments, an alkyl includes C₁-C₅ carbons. In some embodiments, an alkyl includes C₁-C₁₀ carbons. In some embodiments, an alkyl is a C₁-C₁₂ carbons. In some embodiments, an alkyl is a C₁-C₂₀ carbons. In some embodiments, branched alkyl is an alkyl substituted by alkyl side chains of 1 to 5 carbons. In various embodiments, the alkyl group may be unsubstituted. In some embodiments, the alkyl group may be substituted by a halogen, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHCO-alkyl, —C(O)Ph, C(O)O-alkyl, C(O)H, —C(O)NH₂ or any combination thereof.

The alkyl group can be a sole substituent, or it can be a component of a larger substituent, such as in an alkoxy, alkoxyalkyl, haloalkyl, arylalkyl, alkylamino, dialkylamino, alkylamido, alkylurea, etc. Preferred alkyl groups are methyl, ethyl, and propyl, and thus halomethyl, dihalomethyl, trihalomethyl, haloethyl, dihaloethyl, trihaloethyl, halopropyl, dihalopropyl, trihalopropyl, methoxy, ethoxy, propoxy, arylmethyl, arylethyl, arylpropyl, methylamino, ethylamino, propylamino, dimethylamino, diethylamino, methylamido, acetamido, propylamido, halomethylamido, haloethylamido, halopropylamido, methyl-urea, ethyl-urea, propyl-urea, 2, 3, or 4-CH₂—C₆H₄—Cl, C(OH)(CH₃)(Ph), etc.

As used herein, the term “aryl” refers to any aromatic ring that is directly bonded to another group and can be either substituted or unsubstituted. The aryl group can be a sole substituent, or the aryl group can be a component of a larger substituent, such as in an arylalkyl, arylamino, arylamido, etc. In some embodiments, the term aryl according to this invention, includes also heteroaryl. Exemplary aryl groups include, without limitation, phenyl, tolyl, xylyl, furanyl, naphthyl, pyridinyl, pyrimidinyl, pyridazinyl, pyrazinyl, triazinyl, thiazolyl, oxazolyl, isooxazolyl, pyrazolyl, imidazolyl, thiophene-yl, pyrrolyl, indolyl, phenylmethyl, phenylethyl, phenylamino, phenylamido, 3-methyl-4H-1,2,4-triazolyl, oxadiazolyl, 5-methyl-1,2,4-oxadiazolyl, isothiazolyl, thiadiazolyl, triazolyl, etc. Substitutions include but are not limited to: F, Cl, Br, I, C₁-C₅ linear or branched alkyl, C₁-C₅ linear or branched haloalkyl, C₁-C₅ linear or branched alkoxy, C₁-C₅ linear or branched haloalkoxy, CF₃, phenyl, halophenyl, CN, NO₂, —CH₂CN, NH₂, NH-alkyl, N(alkyl)₂, hydroxyl, —OC(O)CF₃, —OCH₂Ph, —NHCO-alkyl, COOH, —C(O)Ph, C(O)O-alkyl, C(O)H, —C(O)NH₂ or any combination thereof.

As used herein, the term “alkoxy” refers to an ether group substituted by an alkyl group as defined above. Alkoxy refers both to linear and to branched alkoxy groups. Nonlimiting examples of alkoxy groups are methoxy, ethoxy, propoxy, iso-propoxy, tert-butoxy.

As used herein, the term “aminoalkyl” refers to an amine group substituted by an alkyl group as defined above. Aminoalkyl refers to monoalkylamine, dialkylamine or trialkylamine. Nonlimiting examples of aminoalkyl groups are —N(Me)₂, —NHMe, —NH₃.

A “haloalkyl” group refers, in some embodiments, to an alkyl group as defined above, which is substituted by one or more halogen atoms, e.g. by F, Cl, Br or I. The term “haloalkyl” include but is not limited to fluoroalkyl, i.e., to an alkyl group bearing at least one fluorine atom. Nonlimiting examples of haloalkyl groups are CF₃, CF₂CF₃, CF₂CH₃, CH₂CF₃, CF₂CH₂CH₃, CH₂CH₂CF₃, CF₂CH(CH₃)₂ and CF(CH₃)—CH(CH₃)₂.

A “halophenyl” group refers, in some embodiments, to a phenyl substitutent which is substituted by one or more halogen atoms, e.g. by F, Cl, Br or I. In one embodiment, the halophenyl is 4-chlorophenyl.

An “alkoxyalkyl” group refers, in some embodiments, to an alkyl group as defined above, which is substituted by alkoxy group as defined above, e.g. by methoxy, ethoxy, propoxy, i-propoxy, t-butoxy etc. Nonlimiting examples of alkoxyalkyl groups are —CH₂—O—CH₃, —CH₂—O—CH(CH₃)₂, —CH₂—O—C(CH₃)₃, —CH₂—CH₂—O—CH₃, —CH₂—CH₂—O—CH(CH₃)₂, —CH₂—CH₂—O—C(CH₃)₃.

A “cycloalkyl” or “carbocyclic” group refers, in various embodiments, to a ring structure comprising carbon atoms as ring atoms, which may be either saturated or unsaturated, substituted or unsubstituted, single or fused. In some embodiments the cycloalkyl is a 3-10 membered ring. In some embodiments the cycloalkyl is a 3-12 membered ring. In some embodiments the cycloalkyl is a 6 membered ring. In some embodiments the cycloalkyl is a 5-7 membered ring. In some embodiments the cycloalkyl is a 3-8 membered ring. In some embodiments, the cycloalkyl group may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHCO-alkyl, —C(O)Ph, C(O)O-alkyl, C(O)H, —C(O)NH₂ or any combination thereof. In some embodiments, the cycloalkyl ring may be fused to another saturated or unsaturated cycloalkyl or heterocyclic 3-8 membered ring. In some embodiments, the cycloalkyl ring is a saturated ring. In some embodiments, the cycloalkyl ring is an unsaturated ring. Non limiting examples of a cycloalkyl group comprise cyclohexyl, cyclohexenyl, cyclopropyl, cyclopropenyl, cyclopentyl, cyclopentenyl, cyclopentadienyl, cyclobutyl, cyclobutenyl, cycloctyl, cycloctadienyl (COD), cycloctaene (COE) etc.

A “heterocycle” or “heterocyclic” group refers, in various embodiments, to a ring structure comprising in addition to carbon atoms, sulfur, oxygen, nitrogen or any combination thereof, as part of the ring. A “heteroaromatic ring” refers in various embodiments, to an aromatic ring structure comprising in addition to carbon atoms, sulfur, oxygen, nitrogen or any combination thereof, as part of the ring. In some embodiments the heterocycle or heteroaromatic ring is a 3-10 membered ring. In some embodiments the heterocycle or heteroaromatic ring is a 3-12 membered ring. In some embodiments the heterocycle or heteroaromatic ring is a 6 membered ring. In some embodiments the heterocycle or heteroaromatic ring is a 5-7 membered ring. In some embodiments the heterocycle or heteroaromatic ring is a 3-8 membered ring. In some embodiments, the heterocycle group or heteroaromatic ring may be unsubstituted or substituted by a halogen, alkyl, haloalkyl, hydroxyl, alkoxy, carbonyl, amido, alkylamido, dialkylamido, cyano, nitro, CO₂H, amino, alkylamino, dialkylamino, carboxyl, thio, thioalkyl, C₁-C₅ linear or branched haloalkoxy, CF₃, phenyl, halophenyl, (benzyloxy)phenyl, —CH₂CN, NH₂, NH-alkyl, N(alkyl)₂, —OC(O)CF₃, —OCH₂Ph, —NHCO-alkyl, —C(O)Ph, C(O)O-alkyl, C(O)H, —C(O)NH₂ or any combination thereof. In some embodiments, the heterocycle ring or heteroaromatic ring may be fused to another saturated or unsaturated cycloalkyl or heterocyclic 3-8 membered ring. In some embodiments, the heterocyclic ring is a saturated ring. In some embodiments, the heterocyclic ring is an unsaturated ring. Non limiting examples of a heterocyclic ring or heteroaromatic ring systems comprise pyridine, piperidine, morpholine, piperazine, thiophene, pyrrole, benzodioxole, benzofuran-2(3H)-one, benzo[d][1,3]dioxole, indole, oxazole, isoxazole, imidazole and 1-methylimidazole, furane, triazole, pyrimidine, pyrazine, oxacyclobutane (1 or 2-oxacyclobutane), naphthalene, tetrahydrothiophene 1,1-dioxide, thiazole, benzimidazole, piperidine, 1-methylpiperidine, isoquinoline, 1,3-dihydroisobenzofuran, benzofuran, 3-methyl-4H-1,2,4-triazole, oxadiazolyl, 5-methyl-1,2,4-oxadiazole, pyrazole, isothiazole, thiadiazole, tetrahydrofurane, oxazolone, oxazolidone, thiazolone, isothiazolinone, isoxazolidinone, imidazolidinone, pyrazolone, 2H-pyrrol-2-one, furanone, thiophenone, thiane 1,1-dioxide, triazolopyrimidine, 6,7-dihydro-5H-pyrazolo[5,1-b][1,3]oxazine or indole.

In some embodiments, “heterocyclic ring” according to this invention refers to substituted or unsubstituted, 3 to 8 membered, saturated, unsaturated or aromatic, single, fused or spiro rings, which comprise at least one heteroatom selected from: N, O or S. In some embodiments, the heterocyclic ring may be substituted, unsubstituted, saturated, unsaturated, aromatic, single, fused or spiro ring; each represent a separate embodiment according to this invention. The heterocyclic ring(s) may be 3-10; 3-9; 3-8; 3-7; 3-6; 3-5; 4-6; 4-7; 4-8; 4-9; 5-6; 5-7; 5-8; 5-10 or 5-9 membered ring(s); each represents a separate embodiment according to this invention. Examples of heterocyclic rings include, but to limited to: pyran, tetrahydropyran, pyrrazole, imidazole, furan, tetrahydrofuran, dioxane, oxetane, azetidine, pyridine, pyridazine, pyrimidine, piperidine, piperazine, triazole, oxadiazole, tetrahydrofuran (THF), piperidine, tetrahydrofurane, morpholine, thiomorpholine 1,1-dioxide, oxa-azaspirodecane, azaspiroheptane, 5-azaspiro[2.4]heptane, 2-azaspiro[3.3]heptane, oxa-azaspiroheptane, 2-oxa-6-azaspiro[3.3]heptane pyrrol, pyrrolidine, pyrrolidine-2-one, 2-oxo-pyrrolidine, pyrrolidinone, quinuclidine, oxetane, azepane, azepan-2-one, azabicyclohexane, 2-azabicyclo[2.1.1]hexane, 3-azabicyclo[3.1.0]hexane, 1-oxa-8-azaspiro[4.5]decane, diazabicyclo[2.2.1]heptane, 2,5-diazabicyclo[2.2.1]heptane, thiomorpholine 1,1-dioxide. In some embodiments, the heterocyclic ring may be further substituted with at least one group selected from: F, Cl, Br, I, CF₃, R₂₀ as defined hereinbelow, C₁-C₅ linear or branched alkyl (e.g., methyl, ethyl, propyl), alkyleneamine (e.g., CH₂—NH₂), C₁-C₅ linear or branched haloalkyl, OH, alkoxy (e.g., OCH₃), alkylene-OH (e.g., CH₂—OH), amide, alkylene-amide (e.g., CH₂—C(O)NH₂), C(O)-heterocyclic ring, amine (e.g., NH₂), alkylamine (e.g., NH(CH₃)), dialkylamine (e.g., N(CH₃)₂), CF₃, aryl, phenyl, halophenyl, heteroaryl, C₃-C₈ cycloalkyl (e.g., cyclopropyl), saturated, unsaturated, aromatic, single fused or spiral 3-8 membered heterocyclic ring, CN, and NO₂; each is a separate embodiment according to this invention.

In some embodiments, “single or fused saturated, unsaturated or aromatic heterocyclic ring” or “saturated, unsaturated, aromatic, single, fused or spiro heterocyclic ring” can be any such ring(s), which comprise at least one heteroatom selected from: N, O or S, including but not limited to: pyridinyl, (2-, 3-, and 4-pyridinyl), quinolinyl, pyrimidinyl, pyridazinyl, pyrazinyl, triazinyl, tetrazinyl, thiazolyl, isothiazolyl, oxazolyl, isoxazolyl, imidazolyl, 1-methylimidazole, pyrazolyl, pyrrolyl, furanyl, thiophene-yl, quinolinyl, isoquinolinyl, 2,3-dihydroindenyl, indenyl, tetrahydronaphthyl, 3,4-dihydro-2H-benzo[b][1,4]dioxepine, benzodioxolyl, benzo[d][1,3]dioxole, tetrahydronaphthyl, indolyl, 1H-indole, isoindolyl, anthracenyl, benzimidazolyl, 2,3-dihydro-1H-benzo[d]imidazolyl, indazolyl, 2H-indazole, triazolyl, 4,5,6,7-tetrahydro-2H-indazole, 3H-indol-3-one, purinyl, benzoxazolyl, 1,3-benzoxazolyl, benzisoxazolyl, benzothiazolyl, 1,3-benzothiazole, 4,5,6,7-tetrahydro-1,3-benzothiazole, quinazolinyl, quinoxalinyl, 1,2,3,4-tetrahydroquinoxaline, 1-(pyridin-1(2H)-yl)ethanone, cinnolinyl, phthalazinyl, quinolinyl, isoquinolinyl, acridinyl, benzofuranyl, 1-benzofuran, isobenzofuranyl, benzofuran-2(3H)-one, benzothiophenyl, benzoxadiazole, benzo[c][1,2,5]oxadiazolyl, benzo[c]thiophenyl, benzodioxolyl, thiadiazolyl, [1,3]oxazolo[4,5-b]pyridine, 1,2,3-, 1,2,4-, 1,2,5- or 1,3,4-oxadiazolyl, imidazo[2,1-b][1,3]thiazole, 4H,5H,6H-cyclopenta[d][1,3]thiazole, 5H,6H,7H,8H-imidazo[1,2-a]pyridine, 7-oxo-6H,7H-[1,3]thiazolo[4,5-d]pyrimidine, [1,3]thiazolo[5,4-b]pyridine, 2H,3H-imidazo[2,1-b][1,3]thiazole, thieno[3,2-d]pyrimidin-4(3H)-one, 4-oxo-4H-thieno[3,2-d][1,3]thiazin, imidazo[1,2-a]pyridine, 1H-imidazo[4,5-b]pyridine, 1H-imidazo[4,5-c]pyridine, 3H-imidazo[4,5-c]pyridine, pyrazolo[1,5-a]pyridine, imidazo[1,2-a]pyrazine, imidazo[1,2-a]pyrimidine, 1H-pyrrolo[2,3-b]pyridine, pyrido[2,3-b]pyrazine, pyrido[2,3-b]pyrazin-3(4H)-one, 4H-thieno[3,2-b]pyrrole, quinoxalin-2(1H)-one, 1H-pyrrolo[3,2-b]pyridine, 7H-pyrrolo[2,3-d]pyrimidine, oxazolo[5,4-b]pyridine, thiazolo[5,4-b]pyridine, thieno[3,2-c]pyridine, 3-methyl-4H-1,2,4-triazole, 5-methyl-1,2,4-oxadiazole etc. In some embodiments, the heterocyclic ring according to this invention includes: pyran, tetrahydropyran, pyrrazole, imidazole, furan, tetrahydrofuran, dioxane, oxetane, azetidine, pyridine, pyridazine, pyrimidine, piperidine, piperazine, triazole, oxadiazole, tetrahydrofuran (THF), piperidine, tetrahydrofurane, morpholine, thiomorpholine 1,1-dioxide, oxa-azaspirodecane, azaspiroheptane, 5-azaspiro[2.4]heptane, 2-azaspiro[3.3]heptane, oxa-azaspiroheptane, pyrrol, pyrrolidine, pyrrolidine-2-one, 2-oxo-pyrrolidine, pyrrolidinone, quinuclidine, oxetane, azepane, azepan-2-one, azabicyclohexane, 2-azabicyclo[2.1.1]hexane, 3-azabicyclo[3.1.0]hexane, 1-oxa-8-azaspiro[4.5]decane, and/or diazabicyclo[2.2.1]heptane; each represent a separate embodiment according to this invention. In some embodiments, the heterocyclic ring may be further substituted with at least one group selected from: F, Cl, Br, I, CF₃, R₂₀ as defined hereinbelow, C₁-C₅ linear or branched alkyl (e.g., methyl, ethyl, propyl), alkyleneamine (e.g., CH₂—NH₂), C₁-C₅ linear or branched haloalkyl, OH, alkoxy (e.g., OCH₃), alkylene-OH (e.g., CH₂—OH), amide, alkylene-amide (e.g., CH₂—C(O)NH₂), C(O)-heterocyclic ring, amine (e.g., NH₂), alkylamine (e.g., NH(CH₃)), dialkylamine (e.g., N(CH₃)₂), CF₃, aryl, phenyl, halophenyl, heteroaryl, C₃-C₈ cycloalkyl (e.g., cyclopropyl), saturated, unsaturated, aromatice, single fused or spiral 3-8 membered heterocyclic ring, CN, and NO₂; each is a separate embodiment according to this invention.

In various embodiments, this invention provides a compound of this invention or its isomer, metabolite, pharmaceutically acceptable salt, pharmaceutical product, tautomer, hydrate, N-oxide, reverse amide analog, prodrug, isotopic variant (deuterated analog), PROTAC, polymorph, or crystal or combinations thereof. In various embodiments, this invention provides an isomer of the compound of this invention. In some embodiments, this invention provides a metabolite of the compound of this invention. In some embodiments, this invention provides a pharmaceutically acceptable salt of the compound of this invention. In some embodiments, this invention provides a pharmaceutical product of the compound of this invention. In some embodiments, this invention provides a tautomer of the compound of this invention. In some embodiments, this invention provides a hydrate of the compound of this invention. In some embodiments, this invention provides an N-oxide of the compound of this invention. In some embodiments, this invention provides a reverse amide analog of the compound of this invention. In some embodiments, “reverse amide analog” refers to acyclic amides or amides of acyclic amines. In some embodiments, this invention provides a prodrug of the compound of this invention. In some embodiments, this invention provides an isotopic variant (including but not limited to deuterated analog) of the compound of this invention. In some embodiments, this invention provides a PROTAC (Proteolysis targeting chimera) of the compound of this invention. In some embodiments, this invention provides a polymorph of the compound of this invention. In some embodiments, this invention provides a crystal of the compound of this invention. In some embodiments, this invention provides composition comprising a compound of this invention, as described herein, or, In some embodiments, a combination of an isomer, metabolite, pharmaceutically acceptable salt, pharmaceutical product, tautomer, hydrate, N-oxide, reverse amide analog, prodrug, isotopic variant (deuterated analog), PROTAC, polymorph, or crystal of the compound of this invention.

In various embodiments, the term “isomer” includes, but is not limited to, stereoisomers including optical isomers and analogs, structural isomers and analogs, conformational isomers and analogs, and the like. In some embodiments, the isomer is a stereoisomer. In another embodiment, the isomer is an optical isomer.

Certain compounds of the present invention may exist in particular geometric or stereoisomeric forms. The present invention contemplates all such compounds, including cis- and trans-isomers, R- and S-enantiomers, diastereomers, the racemic mixtures thereof, and other mixtures thereof, as falling within the scope of the invention. Additional asymmetric carbon atoms may be present in a substituent such as an alkyl group. All such isomers, as well as mixtures thereof, are included in this invention.

In various embodiments, this invention encompasses the use of various stereoisomers of the compounds of the invention. It will be appreciated by those skilled in the art that the compounds of the present invention may contain at least one chiral center. Accordingly, the compounds used in the methods of the present invention may exist in, and be isolated in, optically-active or racemic forms. The compounds according to this invention may further exist as stereoisomers which may be also optically-active isomers (e.g., enantiomers such as (R) or (S)), as enantiomerically enriched mixtures, racemic mixtures, or as single diastereomers, diastereomeric mixtures, or any other stereoisomers, including but not limited to: (R)(R), (R)(S), (S)(S), (S)(R), (R)(R)(R), (R)(R)(S), (R)(S)(R), (S)(R)(R), (R)(S)(S), (S)(R)(S), (S)(S)(R) or (S)(S)(S) stereoisomers. Some compounds may also exhibit polymorphism. It is to be understood that the present invention encompasses any racemic, optically-active, polymorphic, or stereoisomeric form, or mixtures thereof, which form possesses properties useful in the treatment of the various conditions described herein.

It is well known in the art how to prepare optically active forms (for example, by resolution of the racemic form by recrystallization techniques, by synthesis from optically-active starting materials, by chiral synthesis, or by chromatographic separation using a chiral stationary phase).

The compounds of the present invention can also be present in the form of a racemic mixture, containing substantially equivalent amounts of stereoisomers. In some embodiments, the compounds of the present invention can be prepared or otherwise isolated, using known procedures, to obtain a stereoisomer substantially free of its corresponding stereoisomer (i.e., substantially pure). By substantially pure, it is intended that a stereoisomer is at least about 80% pure, more preferably at least about 95% pure, even more preferably at least about 98% pure, most preferably at least about 99% pure.

Compounds of the present invention can also be in the form of a hydrate, which means that the compound further includes a stoichiometric or non-stoichiometric amount of water bound by non-covalent intermolecular forces.

As used herein, when some chemical functional group (e.g., alkyl or aryl) is said to be “substituted”, it is herein defined that one or more substitutions are possible. In some embodiments, the term “substituted” according to this invention, refers to but is not limited to at least one group selected from: halogen, C₁-C₅ linear or branched alkyl, OH, C₁-C₅ linear or branched alkyl-OH (e.g., C(CH₃)₂CH₂—OH, CH₂CH₂—OH), alkoxy (e.g., OMe), amide (e.g., C(O)N(R)₂, C(O)-pyrrolidine, C(O)-piperidine, N(R)₂, NH(R₁₀), N(R₁₀)(R₁₁), (e.g., N(CH₃)₂, NH₂), CF₃, aryl, phenyl, heteroaryl, substituted or unsubstituted C₃-C₈ cycloalkyl (e.g., cyclobutanol), substituted or unsubstituted 3-8 membered heterocyclic ring (e.g. pyran, oxetane, piperidine, pyrazole, methyl-pyrrazole, triazole, imidazole), halophenyl, (benzyloxy)phenyl, CN and NO₂; each represents a separate embodiment according to this invention.

Compounds of the present invention may exist in the form of one or more of the possible tautomers and depending on the conditions it may be possible to separate some or all of the tautomers into individual and distinct entities. It is to be understood that all of the possible tautomers, including all additional enol and keto tautomers and/or isomers are hereby covered. For example, the following tautomers, but not limited to these, are included:

Tautomerization of the Imidazole Ring

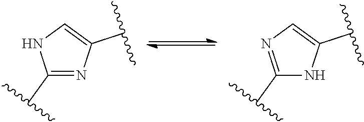 [SMILES: *c1c[nH]c(*)n1 ; *c1cnc(*)[nH]1]

Tautomerization of the Pyrazolone Ring:

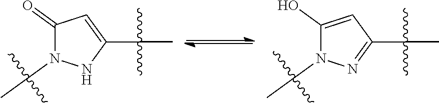 [SMILES: *c1cc(=O)n(*)[nH]1 ; *c1cc(O)n(*)n1]

The invention includes “pharmaceutically acceptable salts” of the compounds of this invention, which may be produced, by reaction of a compound of this invention with an acid or base.

Certain compounds, particularly those possessing acid or basic groups, can also be in the form of a salt, preferably a pharmaceutically acceptable salt. The term “pharmaceutically acceptable salt” refers to those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

Suitable pharmaceutically acceptable salts of amines of compounds the compounds of this invention may be prepared from an inorganic acid or from an organic acid. In various embodiments, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphate, sulfates, sulfamates, sulfanilates, sulfonic acids (alkylsulfonates, arylsulfonates, halogen substituted alkylsulfonates, halogen substituted arylsulfonates), sulfonates and thiocyanates.

In various embodiments, examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, nitrates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

In various embodiments, examples of inorganic salts of carboxylic acids or hydroxyls may be selected from ammonium, alkali metals to include lithium, sodium, potassium, cesium; alkaline earth metals to include calcium, magnesium, aluminium; zinc, barium, cholines, quaternary ammoniums.

In some embodiments, examples of organic salts of carboxylic acids or hydroxyl may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglamines, N-methyl-D-glucamines, N,N′-dibenzylethylenediamines, nicotinamides, organic amines, ornithines, pyridines, picolies, piperazines, procain, tris(hydroxymethyl)methylamines, triethylamines, triethanolamines, trimethylamines, tromethamines and ureas.

In various embodiments, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of a existing salt for another ion or suitable ion-exchange resin.

### Pharmaceutical Composition

Another aspect of the present invention relates to a pharmaceutical composition including a pharmaceutically acceptable carrier and a compound according to the aspects of the present invention. The pharmaceutical composition can contain one or more of the above-identified compounds of the present invention. Typically, the pharmaceutical composition of the present invention will include a compound of the present invention or its pharmaceutically acceptable salt, as well as a pharmaceutically acceptable carrier. The term “pharmaceutically acceptable carrier” refers to any suitable adjuvants, carriers, excipients, or stabilizers, and can be in solid or liquid form such as, tablets, capsules, powders, solutions, suspensions, or emulsions.

Typically, the composition will contain from about 0.01 to 99 percent, preferably from about 20 to 75 percent of active compound(s), together with the adjuvants, carriers and/or excipients. While individual needs may vary, determination of optimal ranges of effective amounts of each component is within the skill of the art. Typical dosages comprise about 0.01 to about 100 mg/kg body wt. The preferred dosages comprise about 0.1 to about 100 mg/kg body wt. The most preferred dosages comprise about 1 to about 100 mg/kg body wt. Treatment regimen for the administration of the compounds of the present invention can also be determined readily by those with ordinary skill in art. That is, the frequency of administration and size of the dose can be established by routine optimization, preferably while minimizing any side effects.

The solid unit dosage forms can be of the conventional type. The solid form can be a capsule and the like, such as an ordinary gelatin type containing the compounds of the present invention and a carrier, for example, lubricants and inert fillers such as, lactose, sucrose, or cornstarch. In some embodiments, these compounds are tabulated with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it can contain, in addition to materials of the above type, a liquid carrier such as a fatty oil.

Various other materials may be present as coatings or to modify the physical form of the dosage unit. For instance, tablets can be coated with shellac, sugar, or both. A syrup can contain, in addition to active ingredient, sucrose as a sweetening agent, methyl and propylparabens as preservatives, a dye, and flavoring such as cherry or orange flavor.

For oral therapeutic administration, these active compounds can be incorporated with excipients and used in the form of tablets, capsules, elixirs, suspensions, syrups, and the like. Such compositions and preparations should contain at least 0.1% of active compound. The percentage of the compound in these compositions can, of course, be varied and can conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 mg and 800 mg of active compound.

The active compounds of the present invention may be orally administered, for example, with an inert diluent, or with an assimilable edible carrier, or they can be enclosed in hard- or soft-shell capsules, or they can be compressed into tablets, or they can be incorporated directly with the food of the diet.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds or pharmaceutical compositions of the present invention may also be administered in injectable dosages by solution or suspension of these materials in a physiologically acceptable diluent with a pharmaceutical adjuvant, carrier or excipient. Such adjuvants, carriers and/or excipients include, but are not limited to, sterile liquids, such as water and oils, with or without the addition of a surfactant and other pharmaceutically and physiologically acceptable components. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

These active compounds may also be administered parenterally. Solutions or suspensions of these active compounds can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

In various embodiments, the compounds of this invention are administered in combination with an anti-cancer therapy. Examples of such therapies include but are not limited to: chemotherapy, immunotherapy, radiotherapy, biological therapy, surgical intervention, and combinations thereof. In various embodiments, the compound is administered in combination with an anti-cancer agent by administering the compounds as herein described, alone or in combination with other agents.

When administering the compounds of the present invention, they can be administered systemically or, alternatively, they can be administered directly to a specific site where cancer is present. Thus, administering can be accomplished in any manner effective for delivering the compounds or the pharmaceutical compositions to the cancerous cells. Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

### Biological Activity

In various embodiments, the invention provides compounds and compositions, including any embodiment described herein, for use in any of the methods of this invention. In various embodiments, use of a compound of this invention or a composition comprising the same, will have utility in inhibiting, suppressing, enhancing, or stimulating a desired response in a subject, as will be understood by one skilled in the art. In some embodiments, the compositions may further comprise additional active ingredients, whose activity is useful for the particular application for which the compound of this invention is being administered.

The invention relates to the treatment, inhibition, and reduction of cancer, employing the use of a compound according to this invention or a pharmaceutically acceptable salt thereof. Accordingly, in various embodiments, this invention is directed to a method of treating, suppressing, reducing the severity, reducing the risk of developing or inhibiting cancer in a subject, comprising administering a compound according to this invention, to a subject suffering from cancer under conditions effective to treat, suppress, reduce the severity, reduce the risk of developing, or inhibit cancer in said subject. In some embodiments, the compound is a c-MYC mRNA translation modulator. In some embodiments, the compound is a c-MYC mRNA translation inhibitor. In some embodiments, the compound is a c-MYC inhibitor. In some embodiments, the compound is a c-MYC mRNA transcription regulator. In some embodiments, the compound is any combination of a c-MYC mRNA transcription regulator, a c-MYC mRNA transcription regulator and a c-MYC inhibitor. In some embodiments, the compound is any one of the compounds listed in Table 1; each compound represents a separate embodiment according to this invention. In some embodiments, the cancer is early cancer. In some embodiments, the cancer is advanced cancer. In some embodiments, the cancer is invasive cancer. In some embodiments, the cancer is metastatic cancer. In some embodiments, the cancer is drug resistant cancer.

In some embodiments, the cancer is selected from the following list: bladder cancer (urothelial carcinoma), myelodysplasia, breast cancer, cervix cancer, endometrium cancer, esophagus cancer, head and neck cancer (squamous cell carcinoma), kidney cancer (e.g., renal cell carcinoma, clear cell renal cell carcinoma), liver cancer (hepatocellular carcinoma), lung cancer (e.g., metastatic, non-small cell, NSCLC, squamous cell carcinoma, small cell (SCLC)), metastatic cancer (e.g., to brain), nasopharynx cancer, solid tumor cancer, stomach cancer, adrenocortical carcinoma, Glioblastoma multiforme, acute myeloid leukemia, chronic lymphocytic leukemia, lymphoma (e.g., Hodgkin's (classical), diffuse large B-cell, primary central nervous system), malignant melanoma, uveal melanoma, meningioma, multiple myeloma, breast cancer, metastatic breast cancer, anus cancer (e.g. squamous cell), biliary cancer, bladder cancer, muscle invasive urothelial carcinoma, colorectal cancer, metastatic colorectal cancer, fallopian tube cancer, gastroesophageal junction cancer (e.g., adenocarcinoma), larynx cancer (e.g., squamous cell), merkel cell cancer, mouth cancer, ovary cancer (e.g., epithelial), pancreas cancer (e.g., adenocarcinoma, metastatic), penis cancer (e.g., squamous cell carcinoma), peritoneum cancer, prostate cancer (e.g., castration-resistant, metastatic), rectum cancer, skin cancer (e.g., basal cell carcinoma, squamous cell carcinoma), small intestine cancer (e.g., adenocarcinoma), testic cancer, thymus cancer, anaplastic thyroid cancer, cholangiocarcinoma, chordoma, cutaneous T-cell lymphoma, digestive-gastrointestinal cancer, familial pheochromocytoma-paraganglioma, Glioma, HTLV-1-associated adult T-cell leukemia-lymphoma, hematologic-blood cancer, hepatitis C (HCV), papillomaviral respiratory Infection, uterine leiomyosarcoma, acute lymphocytic leukemia, chronic myeloid leukemia, T-cell Lymphoma, follicular lymphoma, primary mediastinal large B-cell lymphoma, diffuse large B-cell testicular lymphoma, melanoma, malignant mesothelioma, pleural mesothelioma, mycosis fungoides, neuroendocrine cancer, oral epithelial dysplasia, Sarcoma, severe sepsis, sezary syndrome, smoldering myeloma, soft tissue sarcoma, nasal natural killer (NK) cell T-cell lymphoma, peripheral T-cell lymphoma.

In some embodiments, the cancer is selected from a list including but not limited to: breast cancer, ovarian carcinoma, acute myeloid leukemia, chronic myelogenous leukemia, Hodgkin's and Burkitt's lymphoma, diffuse large Bcell lymphoma, prostate cancer, colon cancer, gastric cancer, primary central nervous system lymphoma, glioblastoma, medulloblastoma, melanoma, non-small cell lung carcinoma, germinal center-derived lymphomas, esophageal squamous cell carcinoma, osteosarcoma, bladder cancer, pancreatic cancer, lung adenocarcinoma, BRAF V600E thyroid cancer, choroid plexus carcinoma, colitis-associated cancer, epithelial ovarian cancer, colorectal cancer, pancreatic cancer and uterine cancer.

In some embodiments, the cancer may be selected from solid tumors and non-solid tumors.

In various embodiments, this invention is directed to a method for suppressing, reducing or inhibiting tumor growth in a subject, comprising administering a compound of this invention, to a subject under conditions effective to suppress, reduce or inhibit tumor growth in said subject.

In some embodiments, the tumor may be a solid tumor or a non-solid tumor.

In some embodiments, the solid tumor cancer is selected from a list including but not limited to: breast cancer, ovarian carcinoma, prostate cancer, colon cancer, gastric cancer, glioblastoma, medulloblastoma, melanoma, non-small cell lung carcinoma, esophageal squamous cell carcinoma, osteosarcoma, bladder cancer, pancreatic cancer, lung adenocarcinoma, BRAF V600E thyroid cancer, choroid plexus carcinoma, colitis-associated cancer, epithelial ovarian cancer, colorectal cancer, pancreatic cancer and uterine cancer.

In some embodiments, the non-solid tumors include but not limited to: hematological malignancies including leukemia, lymphoma or myeloma and inherited cancers such as retinoblastoma and Wilm's tumor.

In some embodiments, the non-solid tumor cancer is selected from a list including but not limited to: acute myeloid leukemia, chronic myelogenous leukemia, Hodgkin's and Burkitt's lymphoma, diffuse large Bcell lymphoma, primary central nervous system lymphoma, glioblastoma, medulloblastoma, germinal center-derived lymphomas, myeloma, retinoblastoma and Wilm's tumor.

Therefore, and in various embodiments, this invention is directed to a method of treating, suppressing, reducing the severity, reducing the risk of developing or inhibiting cancer comprising administering a compound of this invention to a subject suffering from cancer under conditions effective to treat, suppress, reduce the severity, reduce the risk of developing, or inhibit the cancer. In some embodiments, the cancer is early cancer. In some embodiments, the cancer is advanced cancer. In some embodiments, the cancer is invasive cancer. In some embodiments, the cancer is metastatic cancer. In some embodiments, the cancer is drug resistant cancer. In some embodiments, the compound is a c-MYC mRNA translation modulator. In some embodiments, the compound is a c-MYC mRNA translation inhibitor. In some embodiments, the compound is a c-MYC mRNA transcription regulator.

In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting breast cancer comprising administering a compound of this invention to a subject suffering from breast cancer under conditions effective to treat, suppress, reduce the severity, reduce the risk of developing, or inhibit the breast cancer. In some embodiments, the breast cancer is early breast cancer. In some embodiments, the breast cancer is advanced breast cancer. In some embodiments, the breast cancer is invasive breast cancer. In some embodiments, the breast cancer is metastatic breast cancer. In some embodiments, the breast cancer is drug resistant breast cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting ovarian carcinoma comprising administering a compound of this invention to a subject suffering from ovarian carcinoma under conditions effective to treat, suppress, reduce the severity, reduce the risk of developing, or inhibit the ovarian carcinoma. In some embodiments, the ovarian carcinoma is early ovarian carcinoma. In some embodiments, the ovarian carcinoma is advanced ovarian carcinoma. In some embodiments, the ovarian carcinoma is invasive ovarian carcinoma. In some embodiments, the ovarian carcinoma is metastatic ovarian carcinoma. In some embodiments, the ovarian carcinoma is drug resistant ovarian carcinoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting acute myeloid leukemia comprising administering a compound of this invention to a subject suffering from acute myeloid leukemia under conditions effective to treat, suppress, reduce the severity, reduce the risk of developing, or inhibit the acute myeloid leukemia. In some embodiments, the acute myeloid leukemia is early acute myeloid leukemia. In some embodiments, the acute myeloid leukemia is advanced acute myeloid leukemia. In some embodiments, the acute myeloid leukemia is invasive acute myeloid leukemia. In some embodiments, the acute myeloid leukemia is metastatic acute myeloid leukemia. In some embodiments, the acute myeloid leukemia is drug resistant acute myeloid leukemia. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting chronic myelogenous leukemia comprising administering a compound of this invention to a subject suffering from chronic myelogenous leukemia under conditions effective to treat, suppress, reduce the severity, reduce the risk of developing, or inhibit the chronic myelogenous leukemia. In some embodiments, the chronic myelogenous leukemia is early chronic myelogenous leukemia. In some embodiments, the chronic myelogenous leukemia is advanced chronic myelogenous leukemia. In some embodiments, the chronic myelogenous leukemia is invasive chronic myelogenous leukemia. In some embodiments, the chronic myelogenous leukemia is metastatic chronic myelogenous leukemia. In some embodiments, the chronic myelogenous leukemia is drug resistant chronic myelogenous leukemia. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting Hodgkin's and/or Burkitt's lymphoma comprising administering a compound of this invention to a subject suffering from Hodgkin's and/or Burkitt's lymphoma under conditions effective to treat, suppress, reduce the severity, reduce the risk of developing, or inhibit the Hodgkin's and/or Burkitt's lymphoma. In some embodiments, the Hodgkin's and/or Burkitt's lymphoma is early Hodgkin's and/or Burkitt's lymphoma. In some embodiments, the Hodgkin's and/or Burkitt's lymphoma is advanced Hodgkin's and/or Burkitt's lymphoma. In some embodiments, the Hodgkin's and/or Burkitt's lymphoma is invasive Hodgkin's and/or Burkitt's lymphoma. In some embodiments, the cancer is metastatic Hodgkin's and/or Burkitt's lymphoma. In some embodiments, the Hodgkin's and/or Burkitt's lymphoma is drug resistant Hodgkin's and/or Burkitt's lymphoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting diffuse large Bcell lymphoma comprising administering a compound of this invention to a subject suffering from diffuse large Bcell lymphoma under conditions effective to treat, suppress, reduce the severity, reduce the risk of developing, or inhibit the diffuse large Bcell lymphoma. In some embodiments, the diffuse large Bcell lymphoma is early diffuse large Bcell lymphoma. In some embodiments, the diffuse large Bcell lymphoma is advanced diffuse large Bcell lymphoma. In some embodiments, the diffuse large Bcell lymphoma is invasive diffuse large Bcell lymphoma. In some embodiments, the diffuse large Bcell lymphoma is metastatic diffuse large Bcell lymphoma. In some embodiments, the diffuse large Bcell lymphoma is drug resistant diffuse large Bcell lymphoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting prostate cancer comprising administering a compound of this invention to a subject suffering from prostate cancer under conditions effective to treat, suppress, reduce the severity, reduce the risk of developing, or inhibit the prostate cancer. In some embodiments, the prostate cancer is early prostate cancer. In some embodiments, the prostate cancer is advanced prostate cancer. In some embodiments, the prostate cancer is invasive prostate cancer. In some embodiments, the prostate cancer is metastatic prostate cancer. In some embodiments, the prostate cancer is drug resistant prostate cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting colon cancer comprising administering a compound of this invention to a subject suffering from colon cancer under conditions effective to treat, suppress, reduce the severity, reduce the risk of developing, or inhibit the colon cancer. In some embodiments, the colon cancer is early colon cancer. In some embodiments, the colon cancer is advanced colon cancer. In some embodiments, the colon cancer is invasive colon cancer. In some embodiments, the colon cancer is metastatic colon cancer. In some embodiments, the colon cancer is drug resistant colon cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting gastric cancer comprising administering a compound of this invention to a subject suffering from gastric cancer under conditions effective to treat, suppress, reduce the severity, reduce the risk of developing, or inhibit the gastric cancer. In some embodiments, the gastric cancer is early gastric cancer. In some embodiments, the gastric cancer is advanced gastric cancer. In some embodiments, the gastric cancer is invasive gastric cancer. In some embodiments, the gastric cancer is metastatic gastric cancer. In some embodiments, the gastric cancer is drug resistant gastric cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting lymphoma comprising administering a compound of this invention to a subject suffering from lymphoma under conditions effective to treat, suppress, reduce the severity, reduce the risk of developing, or inhibit the lymphoma. In some embodiments, the lymphoma is early lymphoma. In some embodiments, the lymphoma is advanced lymphoma. In some embodiments, the lymphoma is invasive lymphoma. In some embodiments, the lymphoma is metastatic lymphoma. In some embodiments, the lymphoma is drug resistant lymphoma. In some embodiments, the lymphoma is primary central nervous system lymphoma. In some embodiments, the lymphoma is germinal center-derived lymphoma. In some embodiments, the lymphoma is Hodgkin's lymphoma. In some embodiments, the lymphoma is Burkitt's lymphoma. In some embodiments, the lymphoma is diffuse large B-cell lymphoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting glioblastoma comprising administering a compound of this invention to a subject suffering from glioblastoma under conditions effective to treat, suppress, reduce the severity, reduce the risk of developing, or inhibit the glioblastoma. In some embodiments, the glioblastoma is early glioblastoma. In some embodiments, the glioblastoma is advanced glioblastoma. In some embodiments, the glioblastoma is invasive glioblastoma. In some embodiments, the glioblastoma is metastatic glioblastoma. In some embodiments, the glioblastoma is drug resistant glioblastoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting medulloblastoma comprising administering a compound of this invention to a subject suffering from medulloblastoma under conditions effective to treat, suppress, reduce the severity, reduce the risk of developing, or inhibit the medulloblastoma. In some embodiments, the medulloblastoma is early medulloblastoma. In some embodiments, the medulloblastoma is advanced medulloblastoma. In some embodiments, the medulloblastoma is invasive medulloblastoma. In some embodiments, the medulloblastoma is metastatic medulloblastoma. In some embodiments, the medulloblastoma is drug resistant medulloblastoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting melanoma comprising administering a compound of this invention to a subject suffering from melanoma under conditions effective to treat, suppress, reduce the severity, reduce the risk of developing, or inhibit the melanoma. In some embodiments, the melanoma is early melanoma. In some embodiments, the melanoma is advanced melanoma. In some embodiments, the melanoma is invasive melanoma. In some embodiments, the melanoma is metastatic melanoma. In some embodiments, the melanoma is drug resistant melanoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting non-small cell lung carcinoma comprising administering a compound of this invention to a subject suffering from non-small cell lung carcinoma under conditions effective to treat, suppress, reduce the severity, reduce the risk of developing, or inhibit the non-small cell lung carcinoma. In some embodiments, the non-small cell lung carcinoma is early non-small cell lung carcinoma. In some embodiments, the non-small cell lung carcinoma is advanced non-small cell lung carcinoma. In some embodiments, the non-small cell lung carcinoma is invasive non-small cell lung carcinoma. In some embodiments, the non-small cell lung carcinoma is metastatic non-small cell lung carcinoma. In some embodiments, the non-small cell lung carcinoma is drug resistant non-small cell lung carcinoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting esophageal squamous cell carcinoma comprising administering a compound of this invention to a subject suffering from esophageal squamous cell carcinoma under conditions effective to treat, suppress, reduce the severity, reduce the risk of developing, or inhibit the esophageal squamous cell carcinoma. In some embodiments, the esophageal squamous cell carcinoma is early esophageal squamous cell carcinoma. In some embodiments, the esophageal squamous cell carcinoma is advanced esophageal squamous cell carcinoma. In some embodiments, the esophageal squamous cell carcinoma is invasive esophageal squamous cell carcinoma. In some embodiments, the esophageal squamous cell carcinoma is metastatic esophageal squamous cell carcinoma. In some embodiments, the esophageal squamous cell carcinoma is drug resistant esophageal squamous cell carcinoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting osteosarcoma comprising administering a compound of this invention to a subject suffering from osteosarcoma under conditions effective to treat, suppress, reduce the severity, reduce the risk of developing, or inhibit the osteosarcoma. In some embodiments, the osteosarcoma is early osteosarcoma. In some embodiments, the osteosarcoma is advanced osteosarcoma. In some embodiments, the osteosarcoma is invasive osteosarcoma. In some embodiments, the osteosarcoma is metastatic osteosarcoma. In some embodiments, the osteosarcoma is drug resistant osteosarcoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting bladder cancer comprising administering a compound of this invention to a subject suffering from bladder cancer under conditions effective to treat, suppress, reduce the severity, reduce the risk of developing, or inhibit the bladder cancer. In some embodiments, the bladder cancer is early bladder cancer. In some embodiments, the bladder cancer is advanced bladder cancer. In some embodiments, the bladder cancer is invasive bladder cancer. In some embodiments, the bladder cancer is metastatic bladder cancer. In some embodiments, the bladder cancer is drug resistant bladder cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting pancreatic cancer comprising administering a compound of this invention to a subject suffering from pancreatic cancer under conditions effective to treat, suppress, reduce the severity, reduce the risk of developing, or inhibit the pancreatic cancer. In some embodiments, the pancreatic cancer is early pancreatic cancer. In some embodiments, the pancreatic cancer is advanced pancreatic cancer. In some embodiments, the pancreatic cancer is invasive pancreatic cancer. In some embodiments, the pancreatic cancer is metastatic pancreatic cancer. In some embodiments, the pancreatic cancer is drug resistant pancreatic cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting lung adenocarcinoma comprising administering a compound of this invention to a subject suffering from lung adenocarcinoma under conditions effective to treat, suppress, reduce the severity, reduce the risk of developing, or inhibit the lung adenocarcinoma. In some embodiments, the lung adenocarcinoma is early lung adenocarcinoma. In some embodiments, the lung adenocarcinoma is advanced lung adenocarcinoma. In some embodiments, the lung adenocarcinoma is invasive lung adenocarcinoma. In some embodiments, the lung adenocarcinoma is metastatic lung adenocarcinoma. In some embodiments, the lung adenocarcinoma is drug resistant lung adenocarcinoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting thyroid cancer comprising administering a compound of this invention to a subject suffering from thyroid cancer under conditions effective to treat, suppress, reduce the severity, reduce the risk of developing, or inhibit the thyroid cancer. In some embodiments, the thyroid cancer is early thyroid cancer. In some embodiments, the thyroid cancer is advanced thyroid cancer. In some embodiments, the thyroid cancer is invasive thyroid cancer. In some embodiments, the thyroid cancer is metastatic thyroid cancer. In some embodiments, the thyroid cancer is drug resistant thyroid cancer. In some embodiments, the thyroid cancer is BRAF V600E thyroid cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting choroid plexus carcinoma comprising administering a compound of this invention to a subject suffering from choroid plexus carcinoma under conditions effective to treat, suppress, reduce the severity, reduce the risk of developing, or inhibit the choroid plexus carcinoma. In some embodiments, the choroid plexus carcinoma is early choroid plexus carcinoma. In some embodiments, the choroid plexus carcinoma is advanced choroid plexus carcinoma. In some embodiments, the choroid plexus carcinoma is invasive choroid plexus carcinoma. In some embodiments, the choroid plexus carcinoma is metastatic choroid plexus carcinoma. In some embodiments, the choroid plexus carcinoma is drug resistant choroid plexus carcinoma. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting colitis-associated cancer comprising administering a compound of this invention to a subject suffering from colitis-associated cancer under conditions effective to treat, suppress, reduce the severity, reduce the risk of developing, or inhibit the colitis-associated cancer. In some embodiments, the colitis-associated cancer is early colitis-associated cancer. In some embodiments, the colitis-associated cancer is advanced colitis-associated cancer. In some embodiments, the colitis-associated cancer is invasive colitis-associated cancer. In some embodiments, the colitis-associated cancer is metastatic colitis-associated cancer. In some embodiments, the cancer is drug resistant colitis-associated cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting ovarian cancer comprising administering a compound of this invention to a subject suffering from ovarian cancer under conditions effective to treat, suppress, reduce the severity, reduce the risk of developing, or inhibit the ovarian cancer. In some embodiments, the ovarian cancer is early ovarian cancer. In some embodiments, the ovarian cancer is advanced ovarian cancer. In some embodiments, the ovarian cancer is invasive ovarian cancer. In some embodiments, the ovarian cancer is metastatic ovarian cancer. In some embodiments, the ovarian cancer is drug resistant ovarian cancer. In some embodiments, the ovarian cancer is epithelial ovarian cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting colorectal cancer comprising administering a compound of this invention to a subject suffering from colorectal cancer under conditions effective to treat, suppress, reduce the severity, reduce the risk of developing, or inhibit the colorectal cancer. In some embodiments, the colorectal cancer is early colorectal cancer. In some embodiments, the colorectal cancer is advanced colorectal cancer. In some embodiments, the colorectal cancer is invasive colorectal cancer. In some embodiments, the colorectal cancer is metastatic colorectal cancer. In some embodiments, the colorectal cancer is drug resistant colorectal cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention is directed to a method of treating, suppressing, reducing the severity, reducing the risk of developing or inhibiting uterine cancer comprising administering a compound of this invention to a subject suffering from uterine cancer under conditions effective to treat, suppress, reduce the severity, reduce the risk of developing, or inhibit the uterine cancer. In some embodiments, the uterine cancer is early uterine cancer. In some embodiments, the uterine cancer is advanced uterine cancer. In some embodiments, the uterine cancer is invasive uterine cancer. In some embodiments, the uterine cancer is metastatic uterine cancer. In some embodiments, the uterine cancer is drug resistant uterine cancer. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the compound is any one of the compounds listed in Table 1; each compound represents a separate embodiment according to this invention.

In various embodiments, this invention provides methods for increasing the survival of a subject suffering from metastatic cancer comprising the step of administering to said subject a compound of this invention and/or an isomer, metabolite, pharmaceutically acceptable salt, pharmaceutical product, tautomer, hydrate, N-oxide, reverse amide analog, prodrug, isotopic variant (e.g., deuterated analog), PROTAC, polymorph, or crystal of said compound, or any combination thereof. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the cancer is breast cancer, ovarian carcinoma, acute myeloid leukemia, chronic myelogenous leukemia, Hodgkin's and Burkitt's lymphoma, diffuse large Bcell lymphoma, prostate cancer, colon cancer, gastric cancer, primary central nervous system lymphoma, glioblastoma, medulloblastoma, melanoma, non-small cell lung carcinoma, germinal center-derived lymphomas, esophageal squamous cell carcinoma, osteosarcoma, bladder cancer, pancreatic cancer, lung adenocarcinoma, thyroid cancer, choroid plexus carcinoma, colitis-associated cancer, colorectal cancer, or uterine cancer; each represents a separate embodiment according to this invention.

In various embodiments, this invention provides methods for treating, suppressing, reducing the severity, reducing the risk, or inhibiting advanced cancer comprising the step of administering to said subject a compound of this invention and/or an isomer, metabolite, pharmaceutically acceptable salt, pharmaceutical product, tautomer, hydrate, N-oxide, reverse amide analog, prodrug, isotopic variant (e.g., deuterated analog), PROTAC, polymorph, or crystal of said compound, or any combination thereof. In some embodiments, the compound is a c-MYC mRNA translation modulator. In some embodiments, the compound is a c-MYC mRNA translation inhibitor. In some embodiments, the compound is a c-MYC mRNA transcription regulator. In some embodiments, the compound is selective to c-MYC. In some embodiments, the compound reduces the amount of c-Myc protein in a cell. In some embodiments, the cancer is breast cancer, ovarian carcinoma, acute myeloid leukemia, chronic myelogenous leukemia, Hodgkin's and Burkitt's lymphoma, diffuse large Bcell lymphoma, prostate cancer, colon cancer, gastric cancer, primary central nervous system lymphoma, glioblastoma, medulloblastoma, melanoma, non-small cell lung carcinoma, germinal center-derived lymphomas, esophageal squamous cell carcinoma, osteosarcoma, bladder cancer, pancreatic cancer, lung adenocarcinoma, thyroid cancer, choroid plexus carcinoma, colitis-associated cancer, colorectal cancer, or uterine cancer; each represents a separate embodiment according to this invention.

The compounds of the present invention are useful in the treatment, reducing the severity, reducing the risk, or inhibition of cancer, metastatic cancer, advanced cancer, drug resistant cancer, and various forms of cancer. In a preferred embodiment the cancer is breast cancer, ovarian carcinoma, acute myeloid leukemia, chronic myelogenous leukemia, Hodgkin's and Burkitt's lymphoma, diffuse large Bcell lymphoma, prostate cancer, colon cancer, gastric cancer, primary central nervous system lymphoma, glioblastoma, medulloblastoma, melanoma, non-small cell lung carcinoma, germinal center-derived lymphomas, esophageal squamous cell carcinoma, osteosarcoma, bladder cancer, pancreatic cancer, lung adenocarcinoma, thyroid cancer, choroid plexus carcinoma, colitis-associated cancer, colorectal cancer, or uterine cancer; each represents a separate embodiment according to this invention. Based upon their believed mode of action, it is believed that other forms of cancer will likewise be treatable or preventable upon administration of the compounds or compositions of the present invention to a patient. Preferred compounds of the present invention are selectively disruptive to cancer cells, causing ablation of cancer cells but preferably not normal cells. Significantly, harm to normal cells is minimized because the cancer cells are susceptible to disruption at much lower concentrations of the compounds of the present invention.

In various embodiments, other types of cancers that may be treatable with the c-MYC mRNA translation modulators according to this invention include: adrenocortical carcinoma, anal cancer, bladder cancer, brain tumor, brain stem tumor, breast cancer, glioma, cerebellar astrocytoma, cerebral astrocytoma, ependymoma, medulloblastoma, supratentorial primitive neuroectodermal, pineal tumors, hypothalamic glioma, carcinoid tumor, carcinoma, cervical cancer, colon cancer, central nervous system (CNS) cancer, endometrial cancer, esophageal cancer, extrahepatic bile duct cancer, Ewing's family of tumors (Pnet), extracranial germ cell tumor, eye cancer, intraocular melanoma, gallbladder cancer, gastric cancer, germ cell tumor, extragonadal, gestational trophoblastic tumor, head and neck cancer, hypopharyngeal cancer, islet cell carcinoma, laryngeal cancer, leukemia, acute lymphoblastic, leukemia, oral cavity cancer, liver cancer, lung cancer, non-small cell lung cancer, small cell, lymphoma, AIDS-related lymphoma, central nervous system (primary), lymphoma, cutaneous T-cell, lymphoma, Hodgkin's disease, non-Hodgkin's disease, malignant mesothelioma, melanoma, Merkel cell carcinoma, metastatic squamous carcinoma, multiple myeloma, plasma cell neoplasms, mycosis fungoides, myelodysplastic syndrome, myeloproliferative disorders, nasopharyngeal cancer, neuroblastoma, oropharyngeal cancer, osteosarcoma, ovarian cancer, ovarian epithelial cancer, ovarian germ cell tumor, ovarian low malignant potential tumor, pancreatic cancer, exocrine, pancreatic cancer, islet cell carcinoma, paranasal sinus and nasal cavity cancer, parathyroid cancer, penile cancer, pheochromocytoma cancer, pituitary cancer, plasma cell neoplasm, prostate cancer, rhabdomyosarcoma, rectal cancer, renal cancer, renal cell cancer, salivary gland cancer, Sezary syndrome, skin cancer, cutaneous T-cell lymphoma, skin cancer, Kaposi's sarcoma, skin cancer, melanoma, small intestine cancer, soft tissue sarcoma, soft tissue sarcoma, testicular cancer, thymoma, malignant, thyroid cancer, urethral cancer, uterine cancer, sarcoma, unusual cancer of childhood, vaginal cancer, vulvar cancer, Wilms' tumor, hepatocellular cancer, hematological cancer or any combination thereof. In some embodiments the cancer is invasive. In some embodiments the cancer is metastatic cancer. In some embodiments the cancer is advanced cancer. In some embodiments the cancer is drug resistant cancer.

In various embodiments “metastatic cancer” refers to a cancer that spread (metastasized) from its original site to another area of the body. Virtually all cancers have the potential to spread. Whether metastases develop depends on the complex interaction of many tumor cell factors, including the type of cancer, the degree of maturity (differentiation) of the tumor cells, the location and how long the cancer has been present, as well as other incompletely understood factors. Metastases spread in three ways-by local extension from the tumor to the surrounding tissues, through the bloodstream to distant sites or through the lymphatic system to neighboring or distant lymph nodes. Each kind of cancer may have a typical route of spread. The tumor is called by the primary site (ex. breast cancer that has spread to the brain is called metastatic breast cancer to the brain).

In various embodiments “drug-resistant cancer” refers to cancer cells that acquire resistance to chemotherapy. Cancer cells can acquire resistance to chemotherapy by a range of mechanisms, including the mutation or overexpression of the drug target, inactivation of the drug, or elimination of the drug from the cell. Tumors that recur after an initial response to chemotherapy may be resistant to multiple drugs (they are multidrug resistant). In the conventional view of drug resistance, one or several cells in the tumor population acquire genetic changes that confer drug resistance. Accordingly, the reasons for drug resistance, inter alia, are: a) some of the cells that are not killed by the chemotherapy mutate (change) and become resistant to the drug. Once they multiply, there may be more resistant cells than cells that are sensitive to the chemotherapy; b) Gene amplification. A cancer cell may produce hundreds of copies of a particular gene. This gene triggers an overproduction of protein that renders the anticancer drug ineffective; c) cancer cells may pump the drug out of the cell as fast as it is going in using a molecule called p-glycoprotein; d) cancer cells may stop taking in the drugs because the protein that transports the drug across the cell wall stops working; e) the cancer cells may learn how to repair the DNA breaks caused by some anti-cancer drugs; f) cancer cells may develop a mechanism that inactivates the drug. One major contributor to multidrug resistance is overexpression of P-glycoprotein (P-gp). This protein is a clinically important transporter protein belonging to the ATP-binding cassette family of cell membrane transporters. It can pump substrates including anticancer drugs out of tumor cells through an ATP-dependent mechanism; g) Cells and tumors with activating RAS mutations are relatively resistant to most anti-cancer agents. Thus, the resistance to anticancer agents used in chemotherapy is the main cause of treatment failure in malignant disorders, provoking tumors to become resistant. Drug resistance is the major cause of cancer chemotherapy failure.

In various embodiments “resistant cancer” refers to drug-resistant cancer as described herein above. In some embodiments “resistant cancer” refers to cancer cells that acquire resistance to any treatment such as chemotherapy, radiotherapy or biological therapy.

In various embodiments, this invention is directed to treating, suppressing, reducing the severity, reducing the risk, or inhibiting cancer in a subject, wherein the subject has been previously treated with chemotherapy, radiotherapy or biological therapy.

In various embodiments “Chemotherapy” refers to chemical treatment for cancer such as drugs that kill cancer cells directly. Such drugs are referred as “anti-cancer” drugs or “antineoplastics.” Today's therapy uses more than 100 drugs to treat cancer. To cure a specific cancer. Chemotherapy is used to control tumor growth when cure is not possible; to shrink tumors before surgery or radiation therapy; to relieve symptoms (such as pain); and to destroy microscopic cancer cells that may be present after the known tumor is removed by surgery (called adjuvant therapy). Adjuvant therapy is given to prevent a possible cancer reoccurrence.

In various embodiments, “Radiotherapy” (also referred herein as “Radiation therapy”) refers to high energy x-rays and similar rays (such as electrons) to treat disease. Many people with cancer will have radiotherapy as part of their treatment. This can be given either as external radiotherapy from outside the body using x-rays or from within the body as internal radiotherapy. Radiotherapy works by destroying the cancer cells in the treated area. Although normal cells can also be damaged by the radiotherapy, they can usually repair themselves. Radiotherapy treatment can cure some cancers and can also reduce the chance of a cancer coming back after surgery. It may be used to reduce cancer symptoms.

In various embodiments “Biological therapy” refers to substances that occur naturally in the body to destroy cancer cells. There are several types of treatment including: monoclonal antibodies, cancer growth inhibitors, vaccines and gene therapy. Biological therapy is also known as immunotherapy.

When the compounds or pharmaceutical compositions of the present invention are administered to treat, suppress, reduce the severity, reduce the risk, or inhibit a cancerous condition, the pharmaceutical composition can also contain, or can be administered in conjunction with, other therapeutic agents or treatment regimen presently known or hereafter developed for the treatment of various types of cancer. Examples of other therapeutic agents or treatment regimen include, without limitation, radiation therapy, immunotherapy, chemotherapy, surgical intervention, and combinations thereof.

In various embodiments, the compound according to this invention, is administered in combination with an anti-cancer therapy. Examples of such therapies include but are not limited to: chemotherapy, immunotherapy, radiotherapy, biological therapy, surgical intervention, and combinations thereof.

In various embodiments, the compound is administered in combination with an anti-cancer agent by administering the compounds as herein described, alone or in combination with other agents.

In various embodiments, the composition for cancer treatment of the present invention can be used together with existing chemotherapy drugs or be made as a mixture with them. Such a chemotherapy drug includes, for example, alkylating agents, nitrosourea agents, antimetabolites, antitumor antibiotics, alkaloids derived from plant, topoisomerase inhibitors, hormone therapy medicines, hormone antagonists, aromatase inhibitors, P-glycoprotein inhibitors, platinum complex derivatives, other immunotherapeutic drugs, and other anticancer agents. Further, they can be used together with hypoleukocytosis (neutrophil) medicines that are cancer treatment adjuvant, thrombopenia medicines, antiemetic drugs, and cancer pain medicines for patient's QOL recovery or be made as a mixture with them.

In various embodiments, this invention provides a method of modulating c-MYC mRNA translation in a cell, comprising contacting a compound represented by the structure of formula I, II and/or I(a)-I(f) and/or by the structures listed in Table 1, as defined herein above, with a cell, thereby modulating c-MYC mRNA translation in said cell. In some embodiments, the method is carried out by regulating c-MYC mRNA splicing. In some embodiments, the method is carried out by inclusion or exclusion of untranslated region or alternative usage of exons. In some embodiments, the method is carried out by regulation of c-MYC mRNA modifications. In some embodiments, the method is carried out by regulation of the interaction of RNA binding protein with c-MYC mRNA thereby changing mRNA localization. In some embodiments, the method is carried out by regulating c-MYC mRNA localization in the cytoplasm. In some embodiments, the method is carried out by regulating ribosomes or ribosome accessory factor to c-MYC mRNA. In some embodiments, the method is carried out by reducing the amount of c-MYC protein in the cell.

This invention further provides a method of regulating c-MYC mRNA transcription in a cell, comprising contacting a compound represented by the structure of formula I, II and/or I(a)-I(f) and/or by the structures listed in Table 1, as defined herein above, with a cell, thereby regulating c-MYC mRNA transcription in said cell. In some embodiments, the method is carried out by regulating c-MYC mRNA splicing. In some embodiments, the method is carried out by inclusion or exclusion of untranslated region or alternative usage of exons. In some embodiments, the method is carried out by regulation of c-MYC mRNA modifications. In some embodiments, the method is carried out by regulation of the interaction of RNA binding protein with c-MYC mRNA thereby changing mRNA localization. In some embodiments, the method is carried out by regulating c-MYC mRNA localization in the cytoplasm. In some embodiments, the method is carried out by regulating ribosomes or ribosome accessory factor to c-MYC mRNA. In some embodiments, the method is carried out by reducing the amount of c-MYC protein in the cell.

In various embodiments, this invention is directed to a method of destroying a cancerous cell comprising providing a compound of this invention and contacting the cancerous cell with the compound under conditions effective to destroy the contacted cancerous cell. According to various embodiments of destroying the cancerous cells, the cells to be destroyed can be located either in vivo or ex vivo (i.e., in culture).

A still further aspect of the present invention relates to a method of treating or preventing a cancerous condition that includes providing a compound of the present invention and then administering an effective amount of the compound to a patient in a manner effective to treat or prevent a cancerous condition.

According to one embodiment, the patient to be treated is characterized by the presence of a precancerous condition, and the administering of the compound is effective to prevent development of the precancerous condition into the cancerous condition. This can occur by destroying the precancerous cell prior to or concurrent with its further development into a cancerous state.

According to other embodiments, the patient to be treated is characterized by the presence of a cancerous condition, and the administering of the compound is effective either to cause regression of the cancerous condition or to inhibit growth of the cancerous condition, i.e., stopping its growth altogether or reducing its rate of growth. This preferably occurs by destroying cancer cells, regardless of their location in the patient body. That is, whether the cancer cells are located at a primary tumor site or whether the cancer cells have metastasized and created secondary tumors within the patient body.

As used herein, subject or patient refers to any mammalian patient, including without limitation, humans and other primates, dogs, cats, horses, cows, sheep, pigs, rats, mice, and other rodents. In various embodiments, the subject is male. In some embodiments, the subject is female. In some embodiments, while the methods as described herein may be useful for treating either males or females.

When administering the compounds of the present invention, they can be administered systemically or, alternatively, they can be administered directly to a specific site where cancer cells or precancerous cells are present. Thus, administering can be accomplished in any manner effective for delivering the compounds or the pharmaceutical compositions to the cancer cells or precancerous cells.

Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

The following examples are presented in order to more fully illustrate the preferred embodiments of the invention. They should in no way, however, be construed as limiting the broad scope of the invention.

### EXAMPLES

### Example 1

### General Synthetic Details for Compounds of the Invention (Schemes 1-22)

### General Methods

All reagents were commercial grade and were used as received without further purification, unless otherwise specified. Reagent grade solvents were used in all cases, unless otherwise specified. Thin layer chromatography was carried out using pre-coated silica gel F-254 plates (thickness 0.25 mm). ¹H-NMR and ¹⁹F-NMR spectra were recorded on a Bruker Bruker Avance 400 MHz or Avance III 400 MHz spectrometer. The chemical shifts are expressed in ppm using the residual solvent as internal standard. Splitting patterns are designated as s (singlet), d (doublet), dd (doublet of doublets), t (triplet), dt (doublet of triplets), q (quartet), m (multiplet) and br s (broad singlet).

### Abbreviations

AcOH Acetic acid
amphos Bis(di-tert-butyl(4-dimethylaminophenyl)phosphine
Boc tert-Butyloxycarbonyl
BuLi n-butyllithium
t-BuLi tert-butyllithium
DBU 1,8-Diazabicyclo[5.4.0]undec-7-ene
dppb 1,4-Bis(diphenylphosphino)butane
dppf 1,1′-Bis(diphenylphosphino)ferrocene

### DCM Dichloromethane

### DCE 1,2-Dichloroethane

DIBAL-H Diisobutylaluminum hydride

### DIPEA N,N-Diisopropylethylamine

### DMF N,N-Dimethylformamide

### DMA N,N-Dimethylacetamide

### DMAP 4-Dimethylaminopyridine

### DME 1,2-Dimethoxyethane

### DMSO Dimethylsulfonamide

DPPA Diphenyl phosphoryl azide
DTBF 1,1′-Bis(di-tert-butylphosphino)ferrocene
EDC.HCl N-(3-Dimethylaminopropyl)-N′-ethylcarbodiimide hydrochloride
HATU [O-(7-Azabenzotriazol-1-yl)-N,N,N′,N′-tetramethyluronium-hexafluorphosphat]
HPLC High performance liquid chromatography
MsCl Methanesulfonyl chloride

### NBS N-Bromosuccinimide

POBr₃ Phosphorus(V) oxybromide
Py-HBr₃ Pyridinium tribromide

### SEM 2-(Trimethylsilyl)ethoxymethyl

T3P Propylphosphonic anhydride
TBAF Tetrabutylammonium fluoride
TCFH N,N,N,N-tetramethylchloroformamidinium hexafluorophosphate
TFA Trifluoroacetic acid

### THF Tetrahydrofuran

TMS-OTf Trimethylsilyl trifluoromethanesulfonate

### General Synthesis of Compounds of the Invention

### Synthesis of benzo[d]imidazo[2,1-b]thiazole Compounds, Structure I

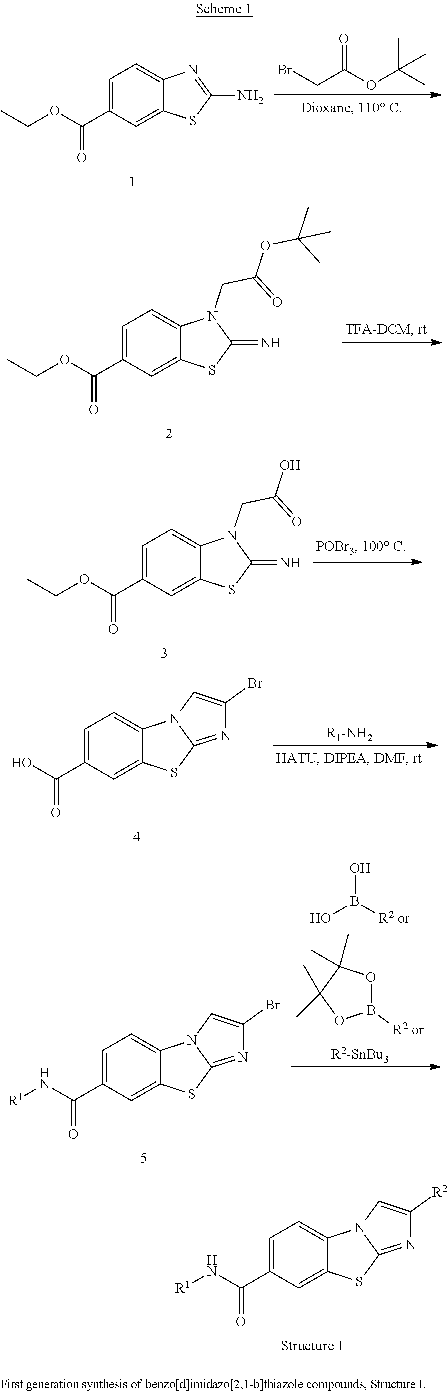

The first step of the synthesis involves alkylation of ethyl 2-aminobenzothiazole-6-carboxylate 1 with tert-butyl bromoacetate at elevated temperature affording alkylated intermediate 2. The tert-butyl group was removed using a mixture of TFA-DCM to generate the carboxylic acid intermediate 3. Treatment of the carboxylic acid intermediate 3 with phosphorus(V) oxybromide at elevated temperature results in intramolecular cyclization to form the benzo[d]imidazo[2,1-b]thiazole intermediate 4. The acid moiety of the left-hand side (LHS) of intermediate 4 was elaborated to the amides, by HATU mediated coupling with a variety of amines affording the amide intermediates 5. The final step of the synthetic sequence involves palladium catalyzed cross-coupling to introduce an aryl/heteroaryl component at the bromo substituent of the heterocyclic intermediate 5. Cross-coupling partners to introduce R² include various boronic acid/esters (Suzuki-Miyaura coupling) or various organostannane reagents (Stille coupling) to furnish the final compounds with various right-hand sides (RHS), Structure I.

### Synthesis of benzo[d]imidazo[2,1-b]thiazole Compounds, Structure II

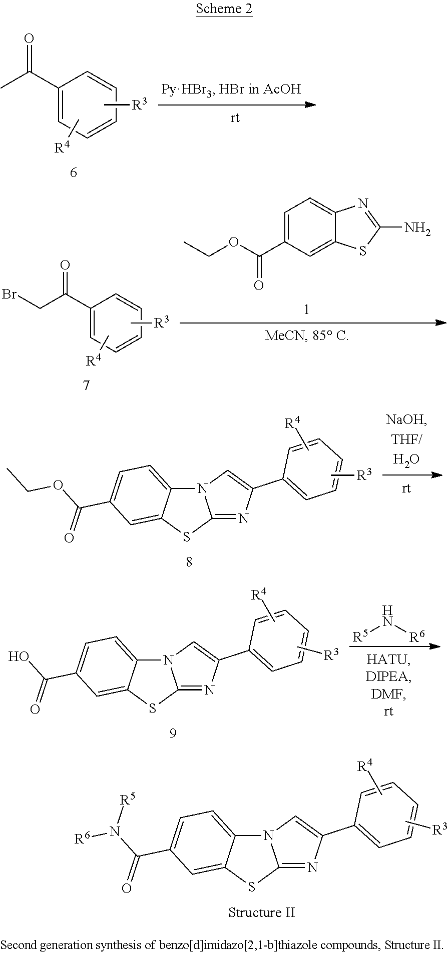

The first step of the synthesis involves bromination of the a-carbonyl position of various substituted aryl methyl ketones 6, using pyridinium tribromide in the presence of HBr in acetic acid affording substituted phenacyl bromide intermediates 7. These intermediates 7 facilitate ready diversification of the right-hand side (RHS) of the final compounds, Structure II. Intermediate 7 undergoes a alkylation reaction followed by intramolecular cyclization with ethyl 2-aminobenzothiazole-6-carboxylate 1 at elevated temperature to from ester benzo[d]imidazo[2,1-b]thiazole intermediate 8. Hydrolysis of ester intermediate 8 with sodium hydroxide in water/THF mixture affords acid intermediate 9. The final step involves an amide coupling of various primary\secondary amines with acid intermediate 9, using HATU as a coupling reagent delivering the final compounds with various left-hand side (LHS) amides, Structure II.

### Alternative Synthesis of benzo[d]imidazo[2,1-b]thiazole Compounds, Structure II

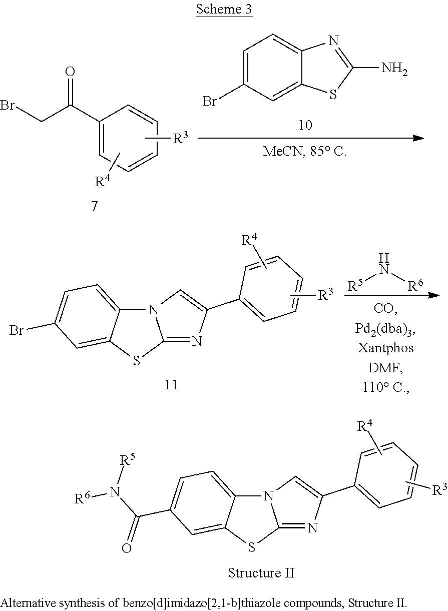

The first step involves a “one-pot” alkylation and intramolecular cyclization reaction between substituted phenacyl bromide intermediates 7 (as in Scheme 2) and 2-amino-6-bromobenzothiazole 10 at elevated temperature affording 7-bromo-2-aryl-lbenzo[d]imidazo[2,1-b]thiazole intermediates 11. The bromo heterocyclic intermediate 11 is employed as the key starting material for the final palladium-catalyzed aminocarbonylation reaction at elevated temperature. Various primary\secondary amines are used in this final palladium-catalyzed aminocarbonylation reaction to provide a variety of left-hand side (LHS) amides, Structure II.

### Synthesis of Reverse amide benzo[d]imidazo[2,1-b]thiazole Compounds, Structure III

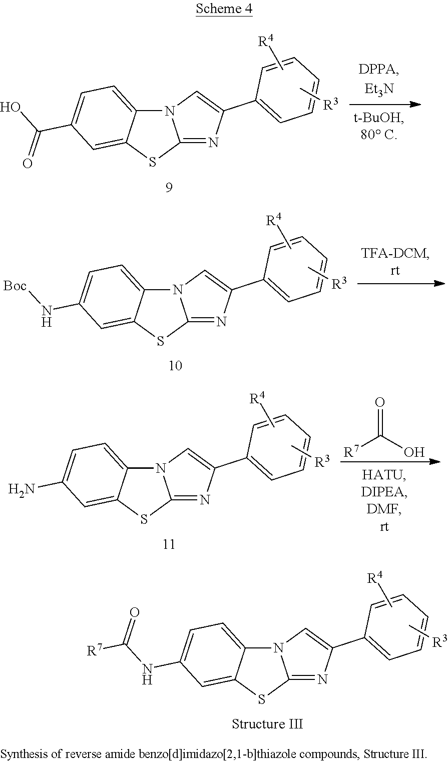

The first step of the synthesis proceeds via a Curtius Rearrangement, using diphenyl phosphoryl azide (DPPA) and tert-butanol in the presence of triethylamine at elevated temperature affording N-Boc amine intermediate 10. N-Boc deprotection of intermediate 10 using a mixture of TFA in DCM enabled ready access to the 7-amino-2-aryl-lbenzo[d]imidazo[2,1-b]thiazole intermediate 11. The final step involves amide coupling of the amine intermediate 11 with a variety of carboxylic acids, using HATU as a coupling reagent to furnish the desired left-hand side (LHS) reverse amides, Structure III.

### Synthesis of 4-(methylcarbamoyl)phenyl)benzo[4,5]imidazo[2,1-b]thiazole-7-carboxamide Compounds, Structure IV

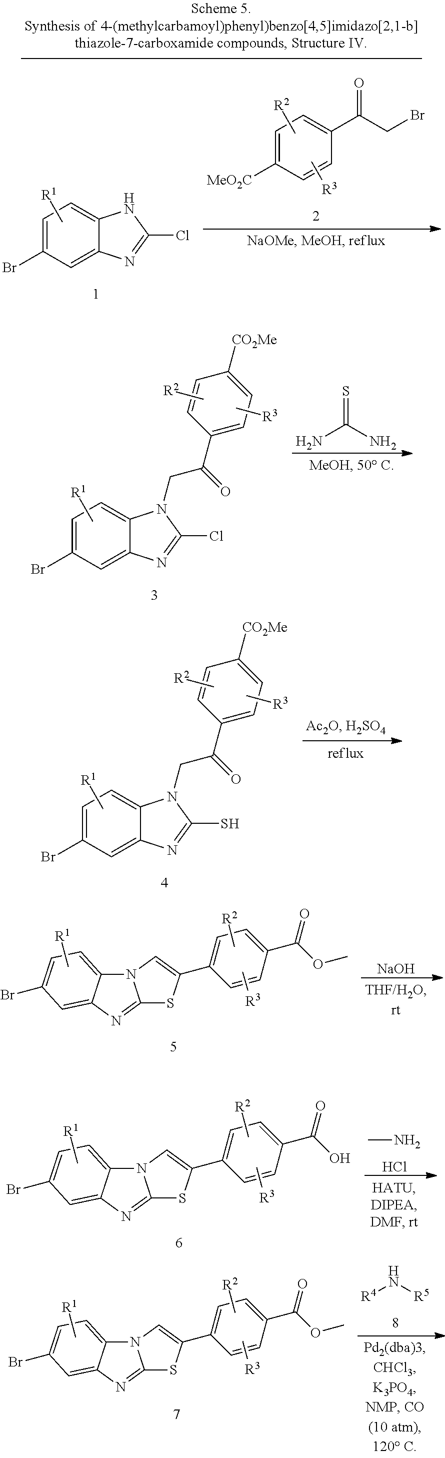

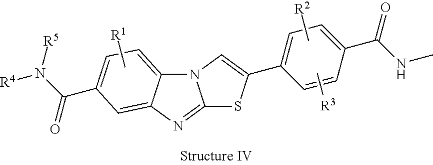

The first step of the synthesis involves alkylation of the R¹ substituted 5-bromo-2-chloro-1H-benzo[d]imidazole 1 with substituted phenacyl bromides 2 affording the N-alkylated intermediates 3. The thiol moiety is introduced by reaction of the 2-chlorobenzimidazole intermediate 3 with thiourea at elevated temperature to form intermediate 4. The third step involves “one pot” acetylation and intramolecular cyclization, using acetic anhydride and sulfuric acid to generate the tricyclic benzo[4,5]imidazo[2,1-b]thiazole ester intermediate 5. Hydrolysis of the methyl ester intermediate 5 using sodium hydroxide in a water/THF mixture gave carboxylic acid intermediate 6. Amide coupling reaction between carboxylic acid intermediate 6 and methylamine hydrochloride, using HATU as a coupling reagent affords the important methylamide intermediate 7. The bromo heteroaryl moiety of intermediate 7 is used in the final palladium-catalyzed aminocarbonylation reaction at elevated temperature with a variety of primary/secondary amines to deliver the final left-hand side (LHS) amides, Structure IV.

### Synthesis of 4-(methylcarbamoyl)phenyl)benzo[4,5]thiazolo[3,2-b][1,2,4]triazole-6-carboxamide Compounds, Structure V

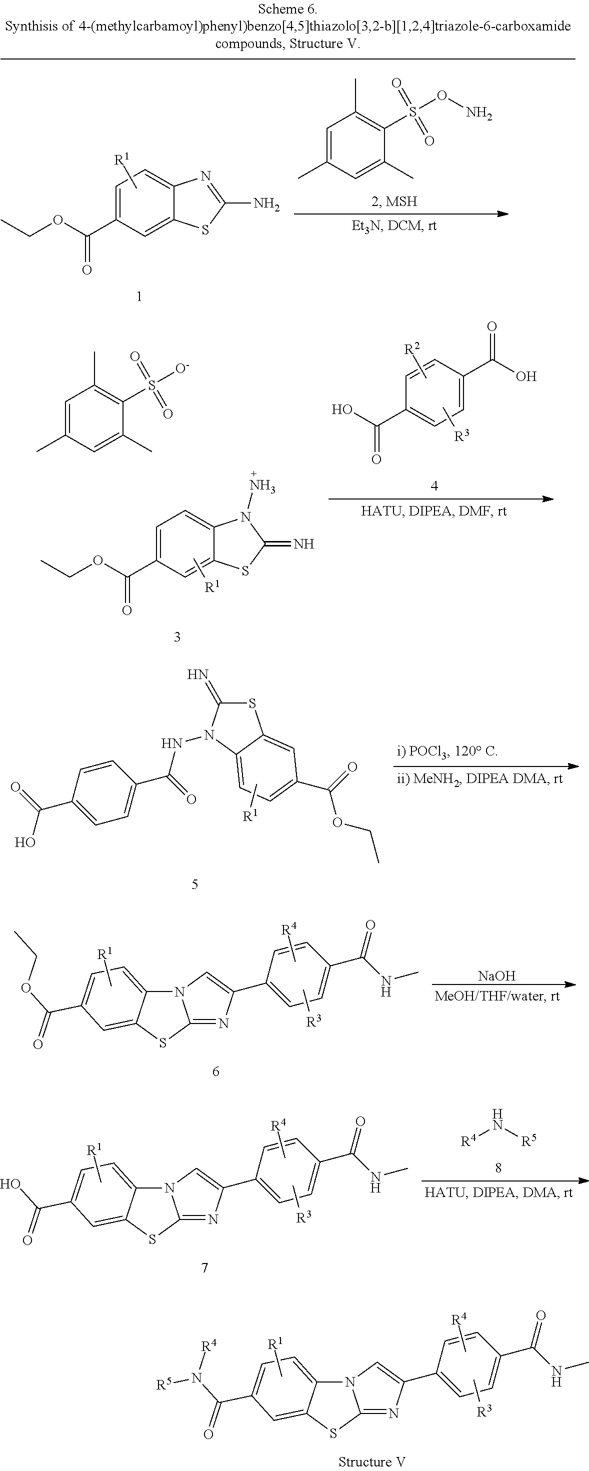

The first step of the synthesis involves electrophilic amination reaction of ethyl 2-aminobenzothiazole-6-carboxylate 1 with O-(2,4,6-trimethylbenzenesulfonyl)hydroxylamine (MSH) 2 in DCM affording the salt intermediate 3. The salt intermediate 3 undergoes an amide coupling reaction with various terephthalic acids 4, using HATU to provide the mono acylated intermediate 5.

Intermediate 5 then undergoes a two-step sequence involving intramolecular cyclization and amidation, using phosphorus(V) oxychloride at elevated temperature followed by treatment with methylamine under basic conditions to afford the 4-(methylcarbamoyl)phenyl)benzo[4,5]thiazolo[3,2-b][1,2,4]triazole intermediate 6. Hydrolysis of the ethyl ester moiety of intermediate 6, using sodium hydroxide in water/THF/MeOH mixture provides the carboxylic acid intermediate 7. The final step involves amide coupling of the carboxylic acid intermediate 7 with a variety of primary/secondary amines, using HATU as a coupling reagent to furnish the final left-hand side (LHS) amides, Structure V.

### Synthesis of 4-(methylcarbamoyl)phenyl)imidazo[2′,1′:2,3]thiazolo[4,5-b]pyridine-7-carboxamide Compounds, Structure VI

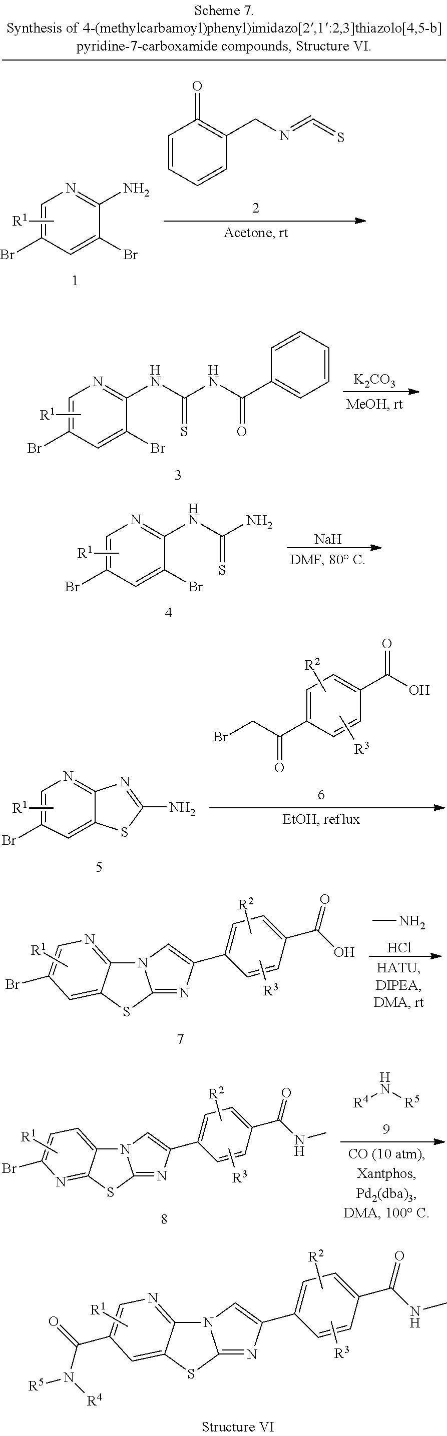

The first step of the synthesis involves reaction of benzoyl isothiocyanate 2 and 2-amino-3,5-dibromopyridine 1 in acetone affording benzoyl thiourea intermediate 3. Base-mediated methanolysis of the benzoyl thiourea intermediate 3 provides thiourea intermediate 4. Subsequently, intramolecular cyclization of thiourea intermediate 4 employing sodium hydride in DMF at elevated temperature furnishes the 6-bromothiazolo[4,5-b]pyridin-2-amine intermediate 5. Step four of the synthesis involves alkylation of the amino moiety of intermediate 5 with 4-carboxylic acid substituted phenacyl bromides 6 followed by intramolecular cyclization in refluxing ethanol to form the imidazothiazolo[4,5-b]pyridine benzoic acid intermediate 7. Amide coupling reaction of the benzoic acid intermediate 7 with methylamine hydrochloride using HATU as the coupling reagent affords the methylamide intermediate 8. In the final step, the 7-bromo heteroaryl moiety of intermediate 8 undergoes a palladium-catalyzed aminocarbonylation reaction at elevated temperature, using various primary/secondary amines to furnish the desired 4-(methylcarbamoyl)phenyl)imidazo[2′,1′:2,3]thiazolo[4,5-b]pyridine-7-carboxamide compounds, Structure VI.

### Synthesis of 4-(methylcarbamoyl)phenyl)imidazo[2′,1′:2,3]thiazolo[5,4-b]pyridine-7-carboxamide Compounds, Structure VII

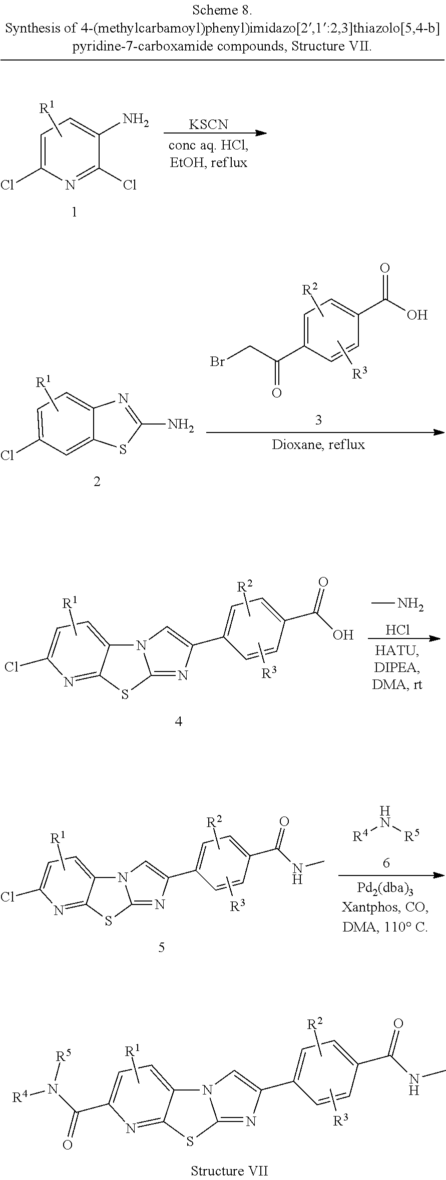

The first step of the synthesis involves reaction of potassium thiocyanate and substituted 2,6-dichloro-3-pyridinamine 1 in refluxing ethanol, in the presence of concentrated aqueous hydrochloric acid affording the 5-chlorothiazolo[5,4-b]pyridin-2-amine intermediate 2. The second step involves alkylation of the amino moiety of intermediate 2 with 4-carboxylic acid substituted phenacyl bromides 3 followed by intramolecular cyclization in refluxing dioxane to form the imidazothiazolo[5,4-b]pyridine benzoic acid intermediate 4. Amide coupling reaction of the benzoic acid intermediate 4 with methylamine hydrochloride, using HATU as the coupling reagent affords the methylamide intermediate 5. In the final step, the 7-chloro heteroaryl moiety of intermediate 5 undergoes a palladium-catalyzed aminocarbonylation reaction at elevated temperature, using various primary/secondary amines to furnish the desired 4-(methylcarbamoyl)phenyl)imidazo[2′,1′:2,3]thiazolo[5,4-b]pyridine-7-carboxamide compounds, Structure VII.

### Synthesis of 4-(methylcarbamoyl)phenyl)imidazo[2′,1′:2,3]thiazolo[5,4-d]pyrimidine-2-carboxamide Compounds, Structure VIII

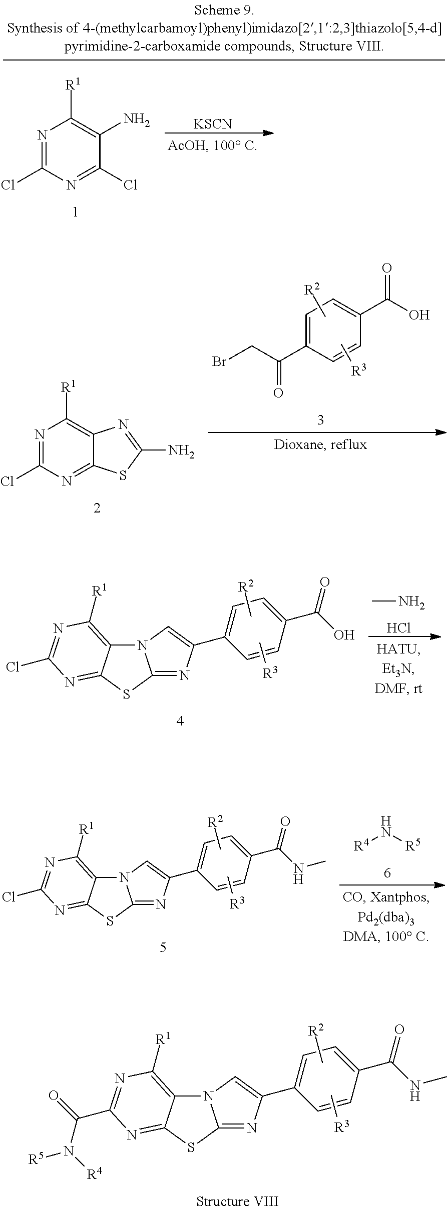

The first step of the synthesis involves reaction of potassium thiocyanate with a 6-substituted 2,4-dichloropyrimidin-5-amine 1 in acetic acid at elevated temperature affording the 5-chlorothiazolo[5,4-d]pyrimidin-2-amine intermediate 2. The second step involves alkylation of the amino moiety of intermediate 2 with 4-carboxylic acid substituted phenacyl bromides 3 followed by intramolecular cyclization in refluxing dioxane to generate the imidazo[2′,1′:2,3]thiazolo[5,4-d]pyrimidin-7-yl)benzoic acid intermediate 4. Amide coupling reaction of the benzoic acid intermediate 4 with methylamine hydrochloride, using HATU as the coupling reagent affords the methylamide intermediate 5. In the final step, the 2-chloroimidazolo moiety of intermediate 5 undergoes a palladium-catalyzed aminocarbonylation reaction at elevated temperature, using various primary/secondary amines to deliver the desired 4-(methylcarbamoyl)phenyl)imidazo[2′,1′:2,3]thiazolo[5,4-d]pyrimidine-2-carboxamide compounds, Structure VIII.

### Synthesis of 4-(methylcarbamoyl)phenyl)imidazo[2′,1′:2,3]thiazolo[4,5-c]pyridine-7-carboxamide Compounds, Structure IX

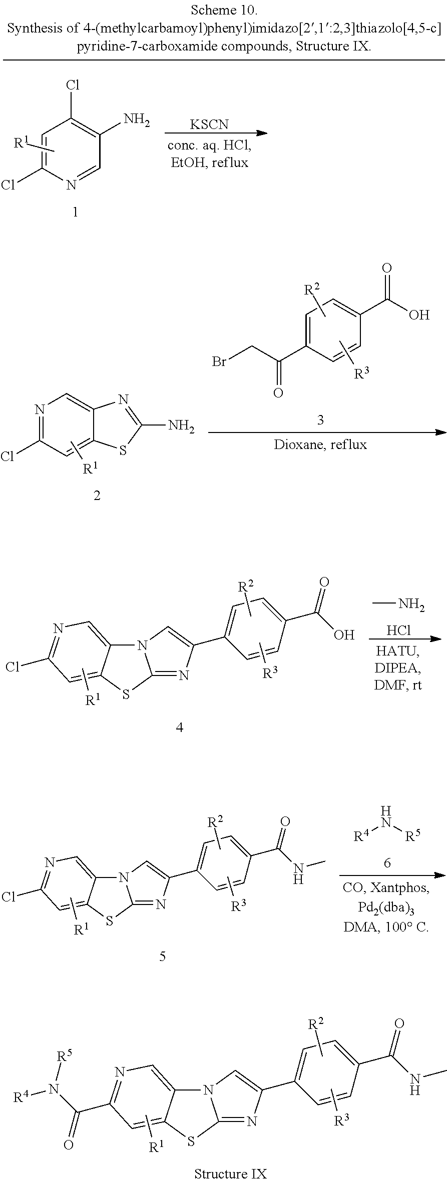

The first step of the synthesis involves reaction of potassium thiocyanate with a substituted 4,6-dichloropyridin-3-amine 1 in refluxing ethanol, in the presence of concentrated aqueous hydrochloric acid affording the 6-chlorothiazolo[4,5-c]pyridin-2-amine intermediate 2. The second step involves alkylation of the amino moiety of intermediate 2 with 4-carboxylic acid substituted phenacyl bromides 3 followed by intramolecular cyclization in refluxing dioxane to generate the imidazo[2′,1′:2,3]thiazolo[4,5-c]pyridin-2-yl)benzoic acid intermediate 4. Amide coupling reaction of the benzoic acid intermediate 4 with methylamine hydrochloride, using HATU as the coupling reagent affords the methylamide intermediate 5. In the final step, the 7-chloro heteroaryl moiety of intermediate 5 undergoes a palladium-catalyzed aminocarbonylation reaction at elevated temperature, using various primary/secondary amines to deliver the desired 4-(methylcarbamoyl)phenyl)imidazo[2′,1′:2,3]thiazolo[4,5-c]pyridine-7-carboxamide compounds, Structure IX.

### First Generation Synthesis of 4-(aminomethyl)phenyl)benzo[4,5]imidazo[2,1-b]thiazole-7-carboxamide Compounds, Structure X

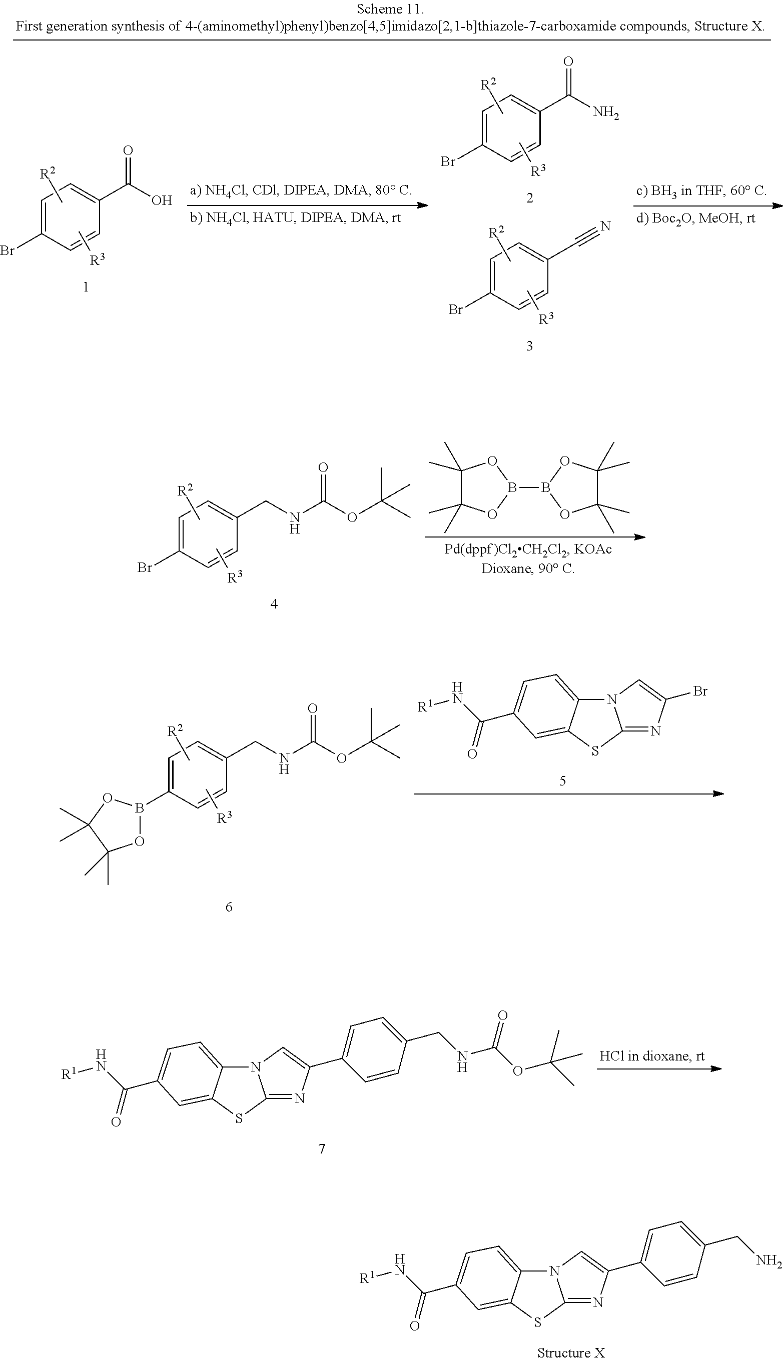

The first step of the synthesis involved primary amide formation from substituted aryl carboxylic acids 1. This was achieved using ammonium chloride and coupling reagents such as CDI or HATU to afford primary amide intermediates 2 and nitrile intermediates 3. Reduction of mixtures of 2 or 3 using borane in THF at elevated temperatures and subsequent protecting group strategy afforded intermediates 4. Palladium-mediated, Miyaura borylation of aryl bromide intermediates 4 gave the desired aryl boronic ester intermediates 6. Intermediates 6 were readily diversified with intermediates 5 to give protected final compounds 7. Acid mediated deprotection of 7 delivered Structure X.

### Second Generation Synthesis of 4-(aminomethyl)phenyl)benzo[4,5]imidazo[2,1-b]thiazole-7-carboxamide Compounds, Structure X

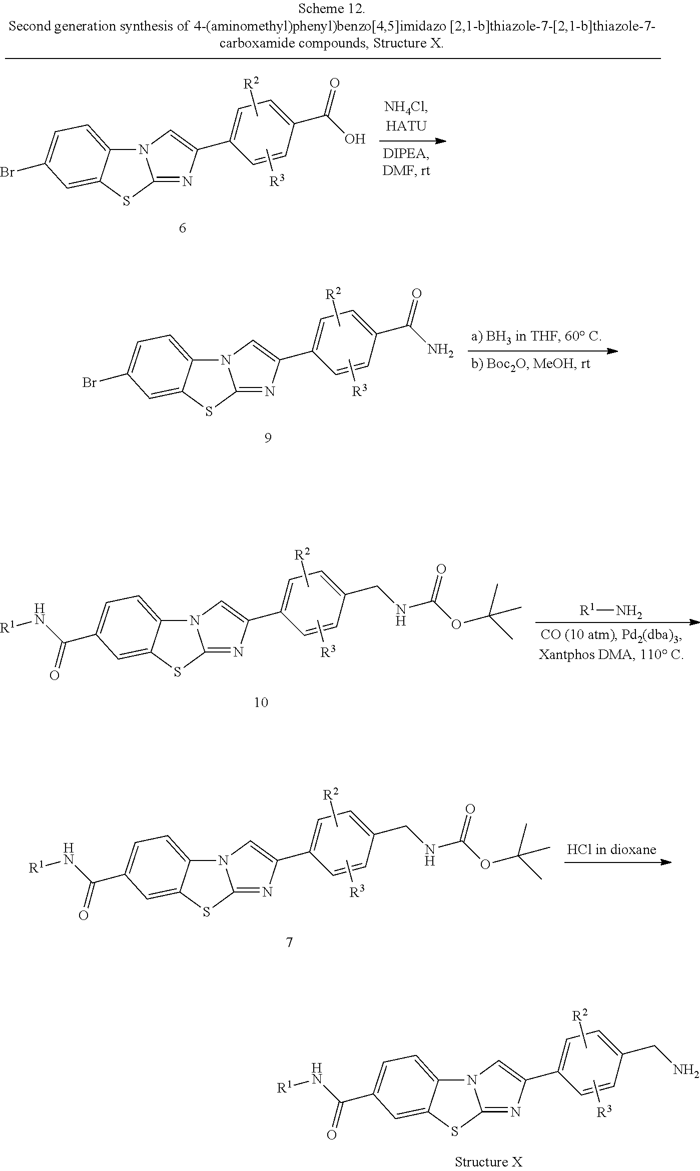

An alternate synthetic sequence involved palladium-catalyzed Suzuki-Miyaura cross-coupling to introduce an aryl/heteroaryl component at the bromo substituted heterocyclic intermediates 5 to generate intermediates 7. The final step of the synthetic sequence involved acid mediated N-Boc deprotection of intermediates 7.

The first step of the synthesis involved primary amide formation from substituted aryl carboxylic acids 6 (as in Scheme 5). This was achieved using ammonium chloride and coupling reagents such as CDI or HATU to afford primary amide intermediates 9. Reduction of intermediates 9 using borane in THF at elevated temperatures and subsequent protecting group strategy afforded intermediates 10. Intermediates 10 were subjected to palladium-catalyzed aminocarbonylation with the desired amine (as in Scheme 3) at elevated temperature to provide intermediates 7. Acid mediated deprotection of intermediates 7 gave final compounds, Structure X.

### Synthesis of 4-(substituted aminomethyl)phenyl)benzo[4,5]imidazo[2,1-b]thiazole-7-carboxamide Compounds, Structure XI

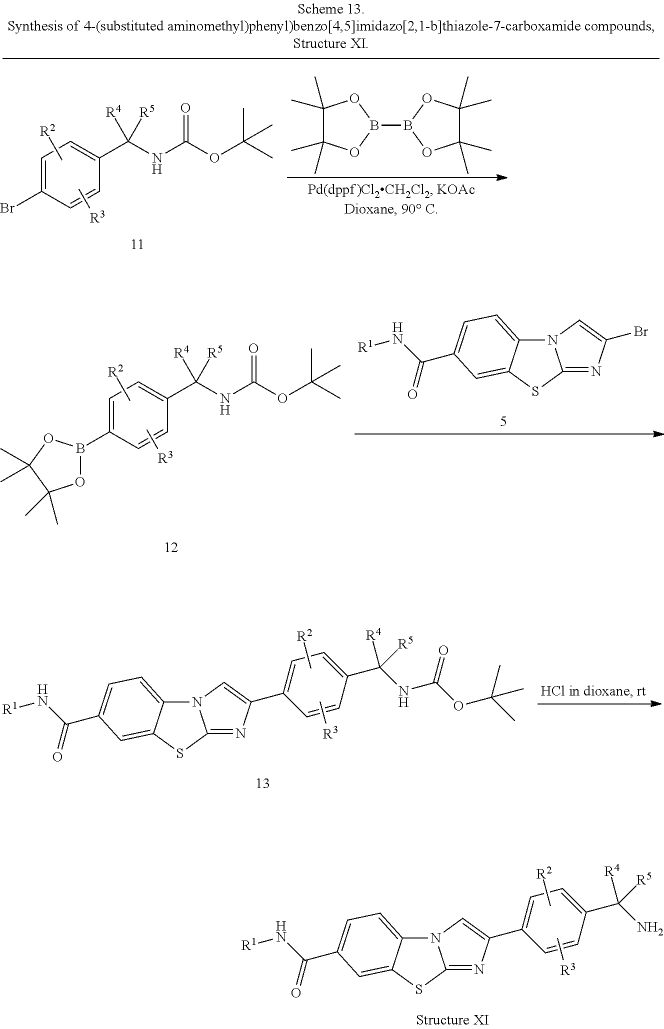

The first step of the synthesis involved palladium-mediated, Miyaura borylation of aryl bromide intermediates 11 to give desired aryl boronic ester intermediates 12. Intermediates 12 undergo palladium-mediated Suzuki-Miyaura cross-coupling, followed by acid mediated N-Boc deprotection reaction to generate the final compounds, Structure XI.

### Synthesis of 4-(N-substituted aminomethyl)phenyl)benzo[4,5]imidazo[2,1-b]thiazole-7-carboxamide Compounds, Structure XII

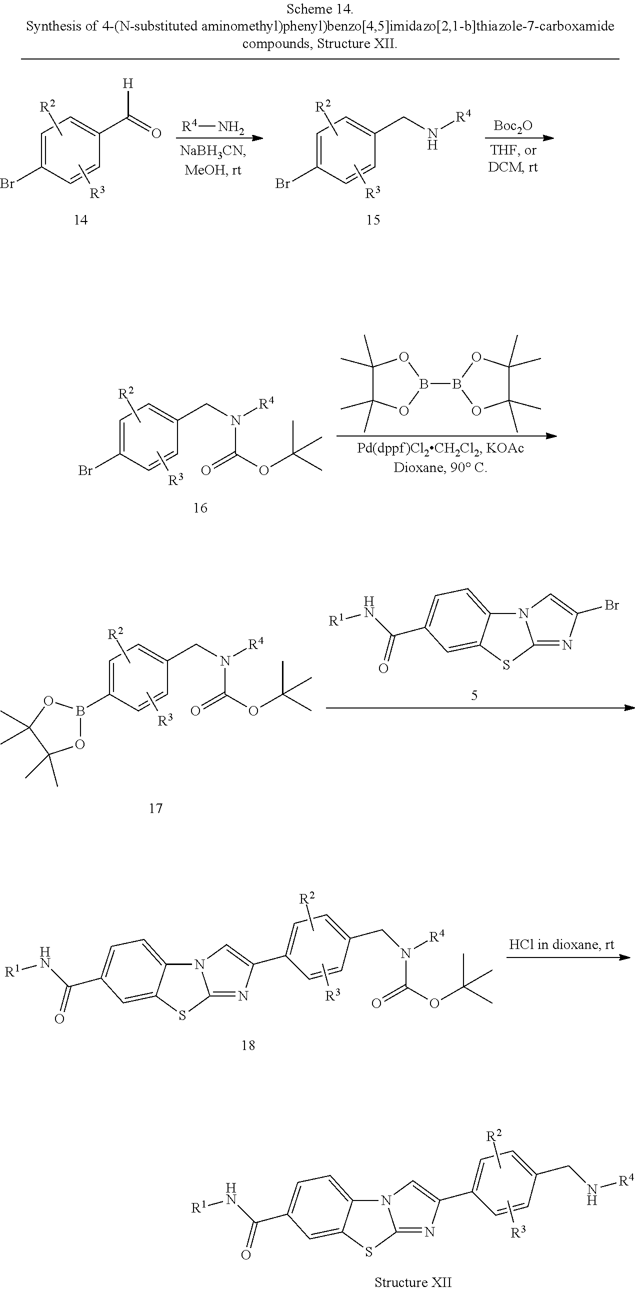

The first step of the synthesis involved reductive amination of aldehyde intermediates 14 with various amines to generate intermediates 15. Intermediates 15 were subsequently protected to give intermediates 16. Intermediates 16 undergo the same synthetic procedure as outlined in Scheme 11 to generate the final compounds, Structure XII.

### Synthesis of 2-(4-(pyrrolidin-2-yl)phenyl)benzo[d]imidazo[2,1-b]thiazole-7-carboxamide Compounds, Structure XIII

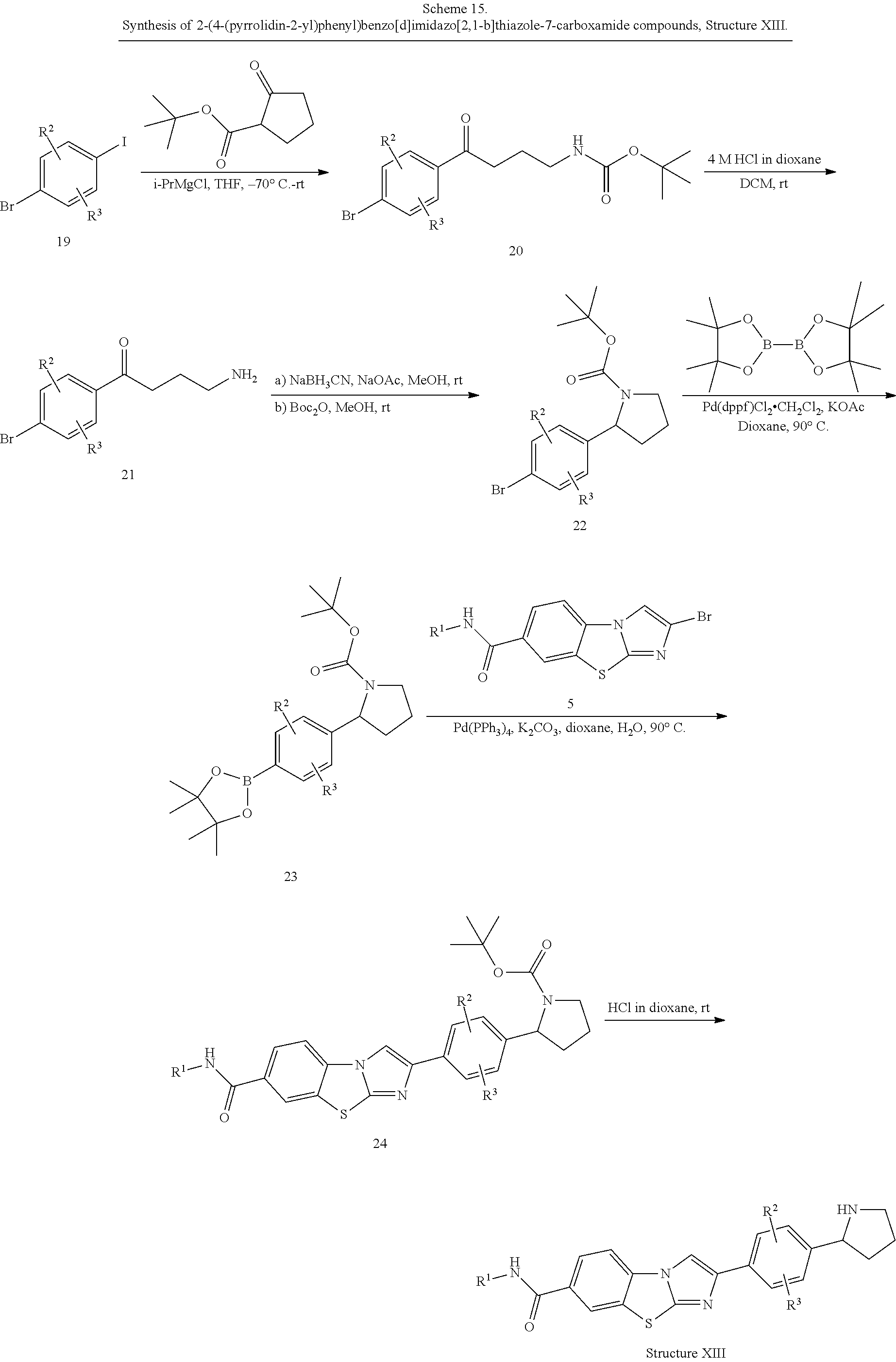

The first step of the synthesis involved Grignard reagent formation of substituted aryl iodide intermediates 19. The resulting Grignard reagents were reacted with tert-butyl 2-oxopyrrolidine-1-carboxylate to give N-Boc aryl ketone intermediates 20. Intermediates 20 are deprotected under acidic conditions to generate intermediates 21. Intermediates 16 undergo the same synthetic procedure as outlined in Scheme 11 to generate final compounds, Structure XIII.

If required, intermediates 24 were separated by chiral HPLC/SFC to generate two enantiomers. The resulting intermediates were deprotected using acidic conditions, to generate the enantiomers of Structure XIII.

### Synthesis of benzo[d]imidazo[2,1-b]thiazole Compounds, Structure IXV

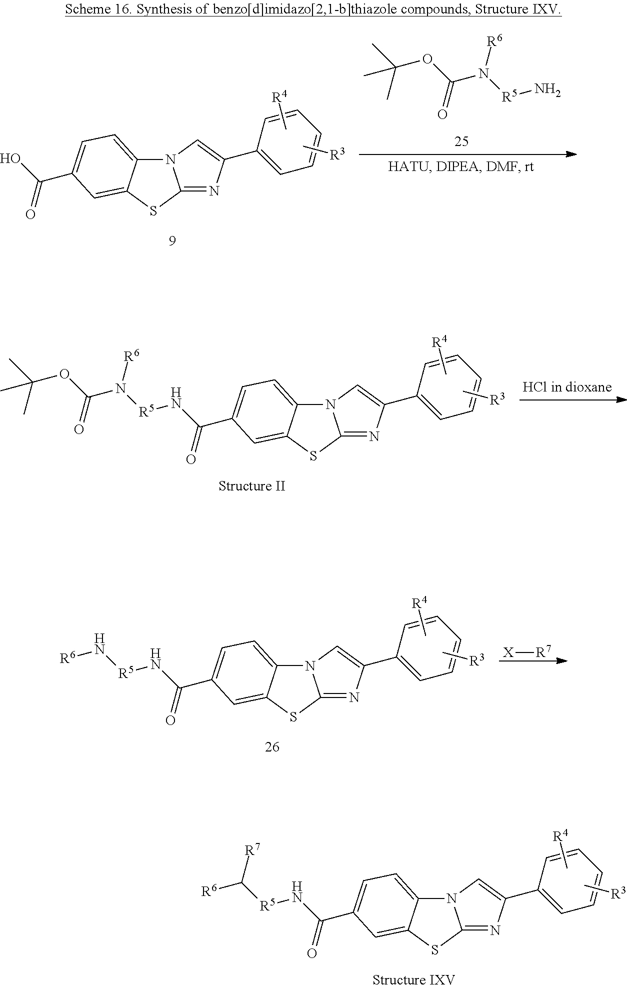

The first step of the synthesis involved amide formation from substituted aryl carboxylic acids 1. Coupling was achieved using reagents such as CDI or HATU and a diverse selection of primary and secondary amines to afford Structure II (as in Scheme 3). Deprotection of Structure II was achieved via acidic conditions (as in Scheme 11) to generate intermediates 26. The final step of the synthesis involved alkylation of intermediates 26 with a variety of alkyl halides to give final compounds of Structure IXV.

### Synthesis of Reverse amide benzo[d]imidazo[2,1-b]thiazole Compounds, Structure XV

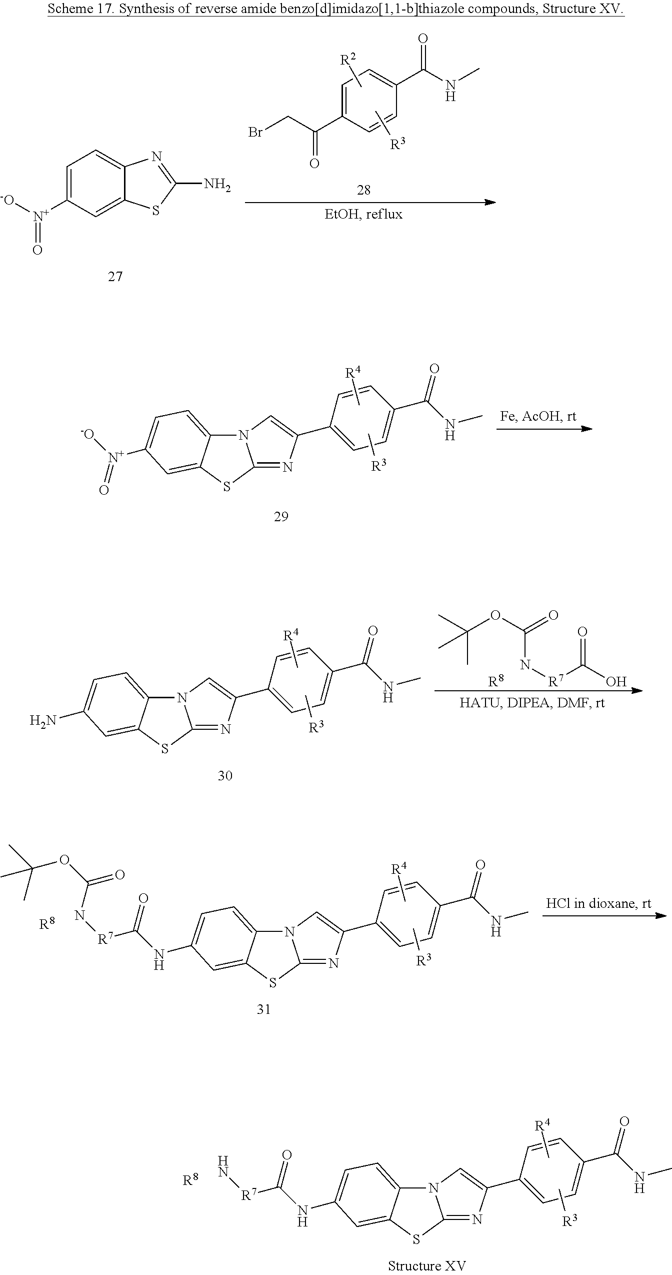

The first step involved a one-pot alkylation, intramolecular cyclization reaction between substituted alpha-bromo ketone intermediates 28 and 6-nitrobenzo[d]thiazol-2-amine 27 at elevated temperature affording intermediates 29. The nitro group was reduced using a mixture of iron in acetic acid to afford intermediates 30. Intermediates 30 were subjected to HATU mediated amide coupling with a variety of carboxylic acids to give intermediates 31. Acid mediated deprotection generated final compounds, Structure XV.

### Alternative Synthesis of 7-nitro-2-aryl-lbenzo[d]imidazo[2,1-b]thiazole Intermediate 29

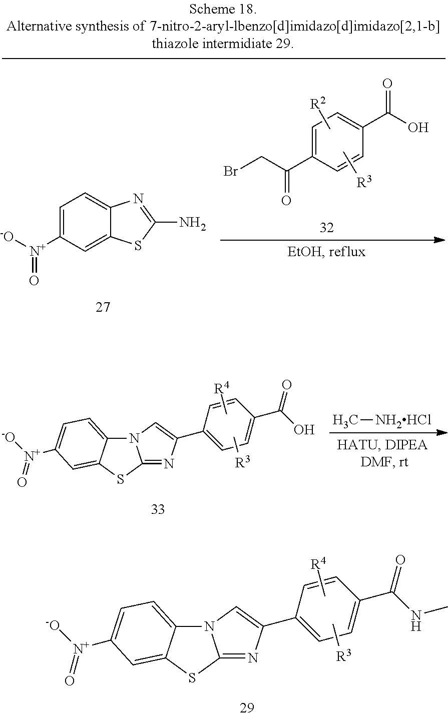

The first step involved a one-pot alkylation, intramolecular cyclization reaction between substituted alpha-bromo ketone intermediates 32 and 6-nitrobenzo[d]thiazol-2-amine 27 at elevated temperature affording intermediates 33. Intermediates 33 were subjected to HATU mediated amide coupling with a methylamine hydrochloride to give intermediate 29.

### Alternative Synthesis of 6-chlorothiazolo[4,5-c]pyridin-2-amine Intermediate 2

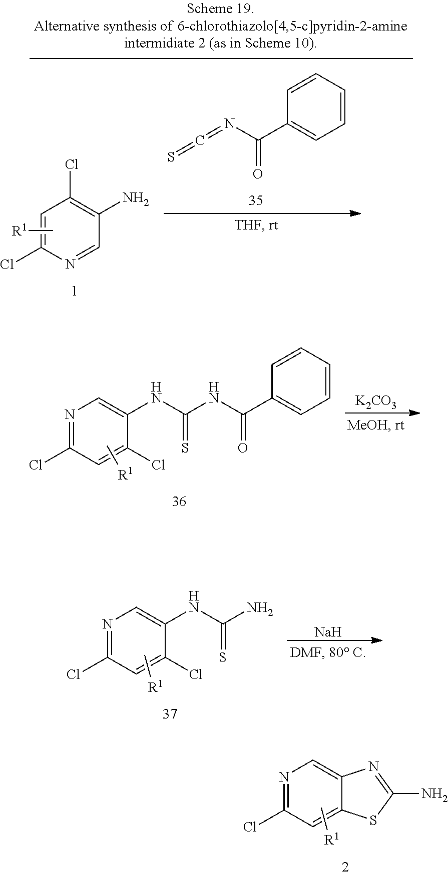

The first step of the synthesis involves reaction of benzoyl isothiocyanate 35 with substituted 4,6-dichloropyridin-3-amines 1 in THF to generate intermediates 36. Base-mediated deprotection of intermediates 36 provided thiourea intermediates 37. Intermediates 37 were subjected to intramolecular cyclization mediated by sodium hydride in DMF at elevated temperature to afford intermediates 2 (as in Scheme 10).

### Synthesis of 4-(substituted aminomethyl)phenyl)benzo[4,5]thiazolo[3,2-b][1,2,4]triazole-6-carboxamide Compounds, Structure XVI

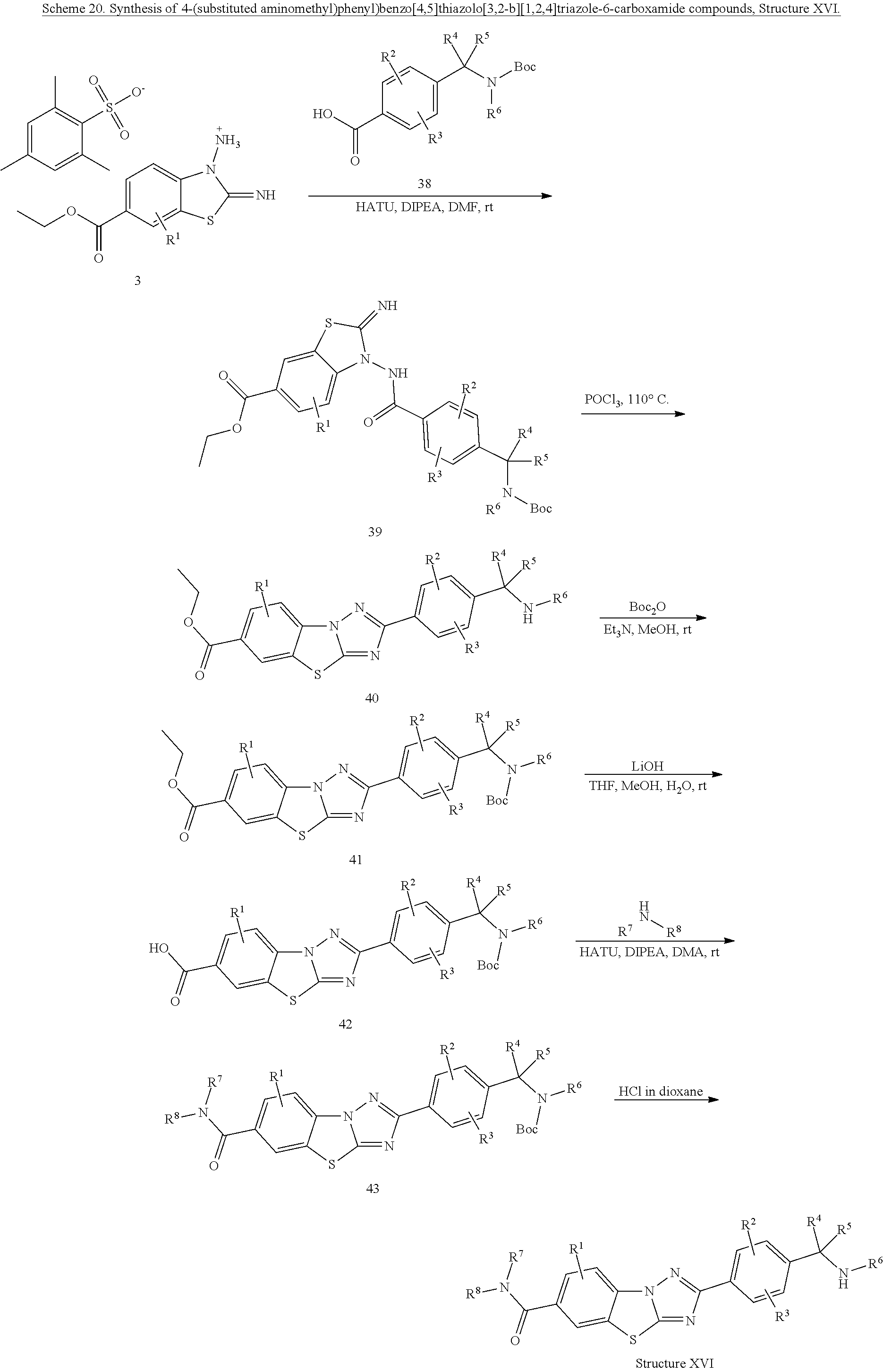

The first step of the synthesis intermediates 3 were subjected to HATU mediated amide coupling with a variety of carboxylic acids to give intermediates 39. Intermediates 39 were subjected to intramolecular cyclization, using phosphorus(V) oxychloride at elevated temperature to generate intermediates 40. Intermediates 40 were then subsequently treated with Boc₂O under basic conditions to give intermediates 41. Hydrolysis of ester intermediates 41 with lithium hydroxide in a mixture of water/THF/MeOH afforded carboxylic acid intermediates 42. Intermediates 42 were subjected to HATU mediated amide coupling with a diverse range of primary/secondary amines, to generate intermediates 43. Acid mediated deprotection reaction gave compounds, Structure XVI.

If required, intermediates 43 were separated by chiral HPLC/SFC to generate two enantiomers. The resulting intermediates were deprotected using acidic conditions, to generate the enantiomers of Structure XVI.

### Second Generation Synthesis of Intermediates 41

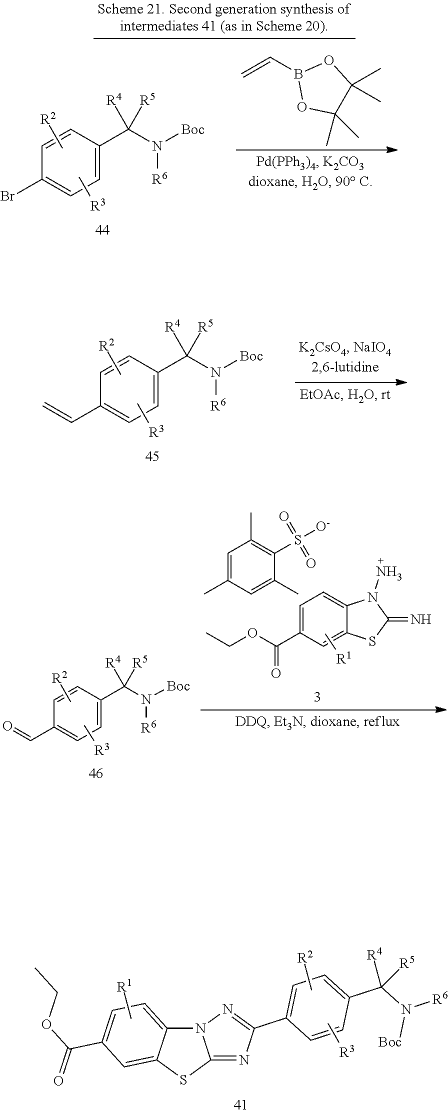

The first step of the synthesis involved a palladium-mediated Suzuki-Miyaura coupling reaction to introduce a vinyl substituent on intermediate 44 to generate intermediate 45. Intermediate 45 is subjected to oxidation to generate aldehyde intermediates 46. The final step of the synthesis involved an oxidative intermolecular cyclization between intermediates 46 and intermediate 3 to give ester intermediates 41.

### Alternative Synthesis of Intermediates 46

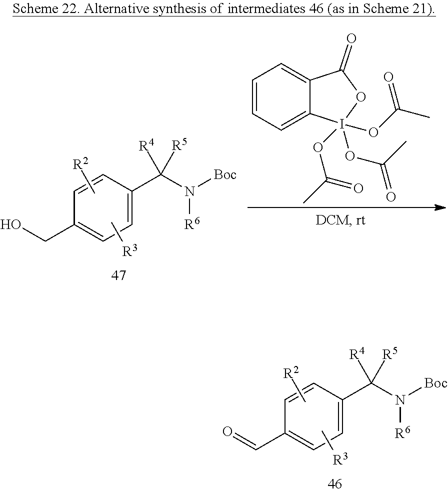

This step of the synthesis involved oxidation of benzyl alcohol intermediates 47 using Dess-Martin periodinane or other oxidants to generate aldehyde intermediates 46.

### Example 2

### General Synthetic Details for Additional Compounds of the Invention (Schemes 23-30)

### General Synthesis of Additional Compounds of the Invention

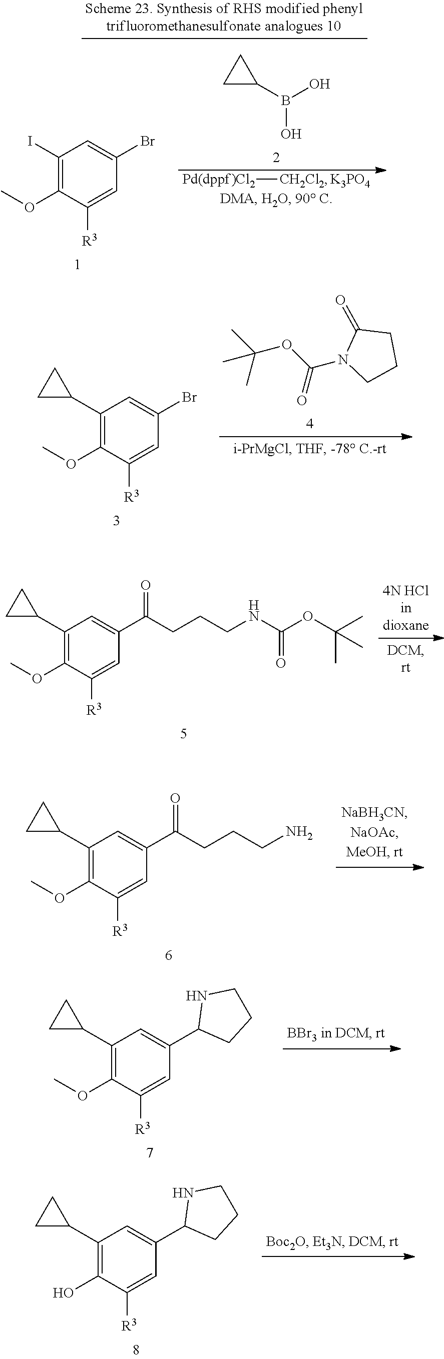

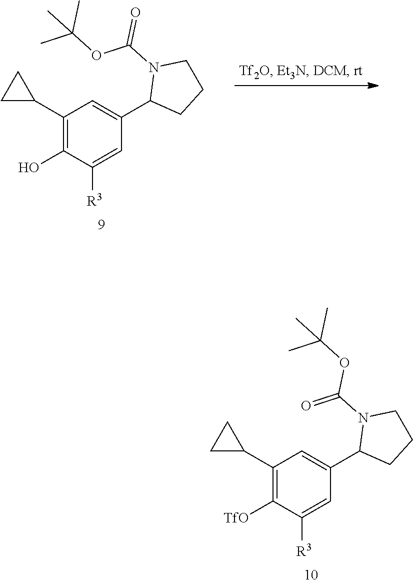

The first step of the synthesis involved a Suzuki coupling reaction between substituted iodobenzenes 1 and cyclopropylboronic acid 2 to afford intermediates 3. Halogen magnesium exchange of intermediates 3 by isopropylmagnesium bromide at a lower temperature formed new aryl magnesium reagents, which were treated with tert-butyl 2-oxopyrrolidine-1-carboxylate 4 to generate aryl alkyl ketones 5. Subsequently the N-Boc group was removed under acidic conditions to give amine intermediates 6 as hydrochloride salts or free bases. Intermediates 6 were subjected to an intramolecular reductive amination reaction to generate intermediates 7. The methoxy protecting groups of aryl methyl ethers were removed using boron tribromide to give amino poly-substituted phenols 8. Intermediates 8 were subjected to amino group protection with Boc groups to generate intermediates 9. In the final step the hydroxyl groups were converted to the corresponding trifluoromethanesulfonates by treatment with trifluoromethanesulfonic anhydride under basic conditions to give elaborated intermediates 10 (Scheme 23).

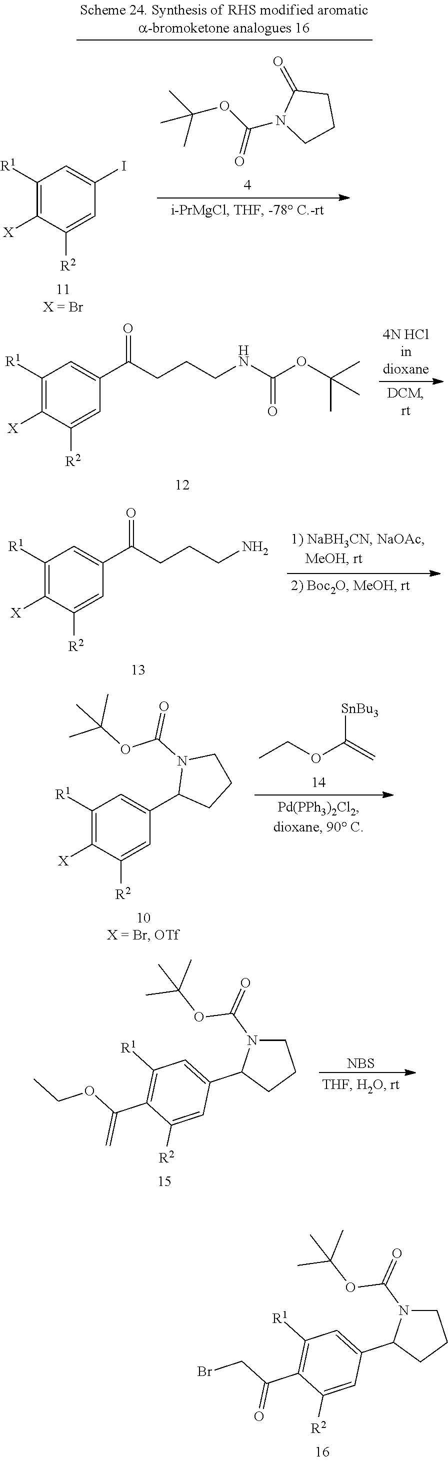

The first 3 steps were conducted using similar methodology the corresponding steps described in Scheme 23 to generate intermediates 10. Subsequently a 1-ethoxyvinyl group was introduced via Stille coupling of intermediates 10 with tributyl(1-ethoxyvinyl)stannane 14 affording intermediates 15. Intermediates 15 were treated with NBS in THF and water to give the corresponding aromatic a-bromoketones 16 (Scheme 24).

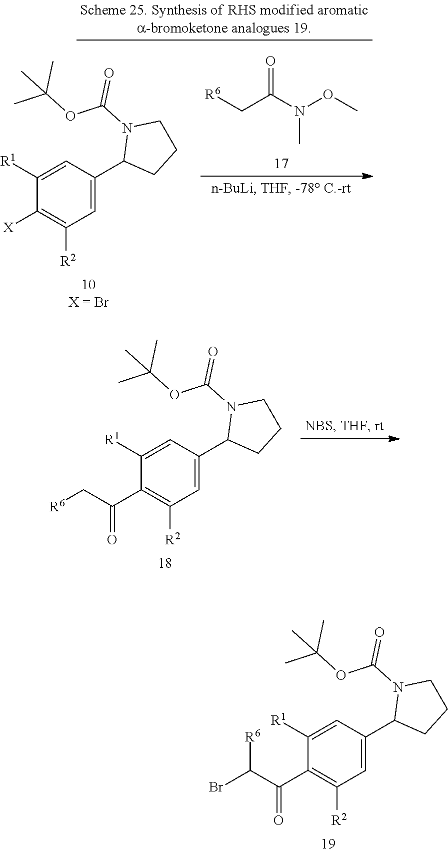

The corresponding step involved lithium halogen exchange of intermediates 10 with n-butyl lithium, followed by quenching with substituted Weinreb amides 17 forming a-substituted aromatic ketone intermediates 18. Subsequent bromination at a-position of ketone intermediates 18 with brominating reagents such as NBS generated modified a-bromoketones 19 (Scheme 25).

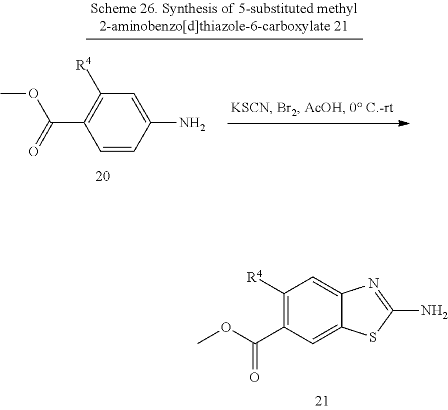

Intermediates 20 were treated with potassium thiocyanate and bromine in acetic acid to generate corresponding 2-aminobenzo[d]thiazoles 21 (Scheme 26).

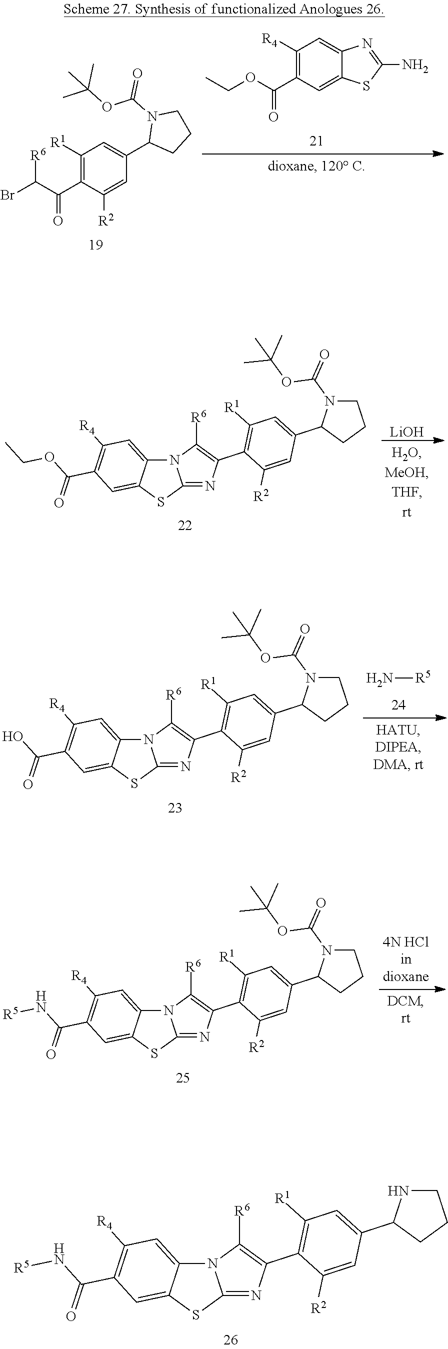

The initial step involved a cyclization reaction between modified a-bromoketone analogues 19 and corresponding 2-aminobenzo[d]thiazole 21 affording substituted tricyclic benzo[d]imidazo[2,1-b]thiazole intermediates 22. The carboxylic esters were hydrolyzed under basic conditions such as aqueous lithium hydroxide to afford carboxylic acids 23. Subsequent condensation reaction of intermediates 23 with corresponding amines afforded N-Boc protected amides 25. Intermediates 25 were deprotected under acidic conditions such as hydrogen chloride in dioxane solution to generate final compounds 26 (Scheme 27).

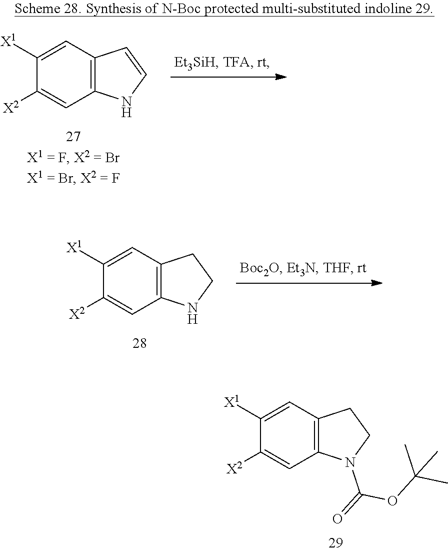

Substituted indoles 27 were reduced with triethylsilane under acidic conditions to afford indolines 28, which were Boc protected to generate corresponding intermediates 29 (Scheme 28).

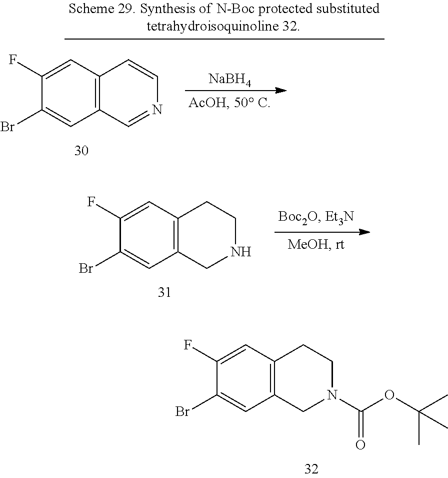 [SMILES: Fc1cc2ccncc2cc1Br ; "NaBH4" ; Fc1cc2c(cc1Br)CNCC2 ; CC(C)(C)OC(=O)N1CCc2cc(F)c(Br)cc2C1]

Substituted isoquinoline 30 was reduced to 1,2,3,4-tetrahydroisoquinoline 31. The secondary amine was Boc group protected to generate corresponding intermediate 32 (Scheme 29).

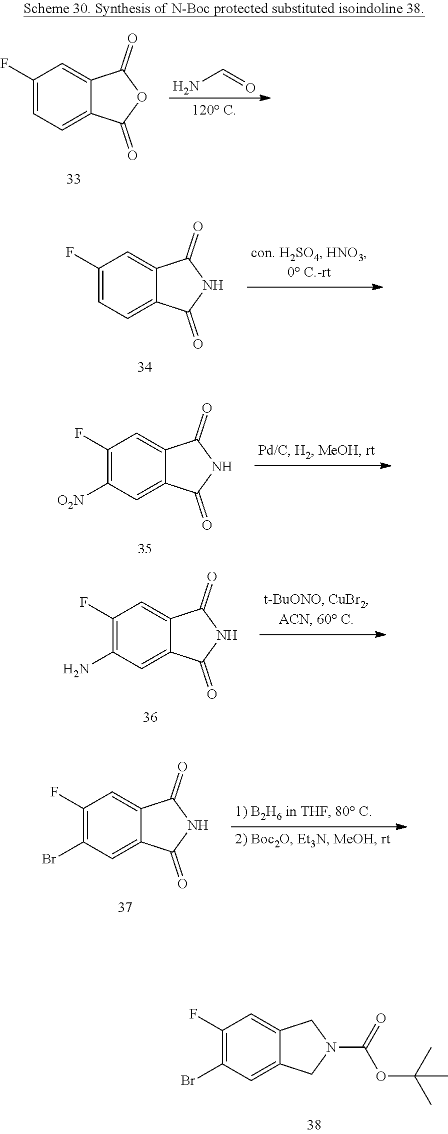

The first step of the synthesis involved reaction of 5-fluoroisobenzofuran-1,3-dione in formamide at elevated temperatures affording 5-fluoroisoindoline-1,3-dione intermediate 33. Intermediate 33 was subjected to regioselective nitration to generate intermediate 35. The nitro group was reduced using 10% palladium on activated carbon to give intermediate 36. Intermediate 36 was treated with t-butyl nitrite and copper(I) bromide to generate intermediate 37. The final step of the synthesis involved reduction of intermediate 37 with diborane in THF at elevated temperatures following by in-situ Boc protection affording intermediate 38 (Scheme 30).

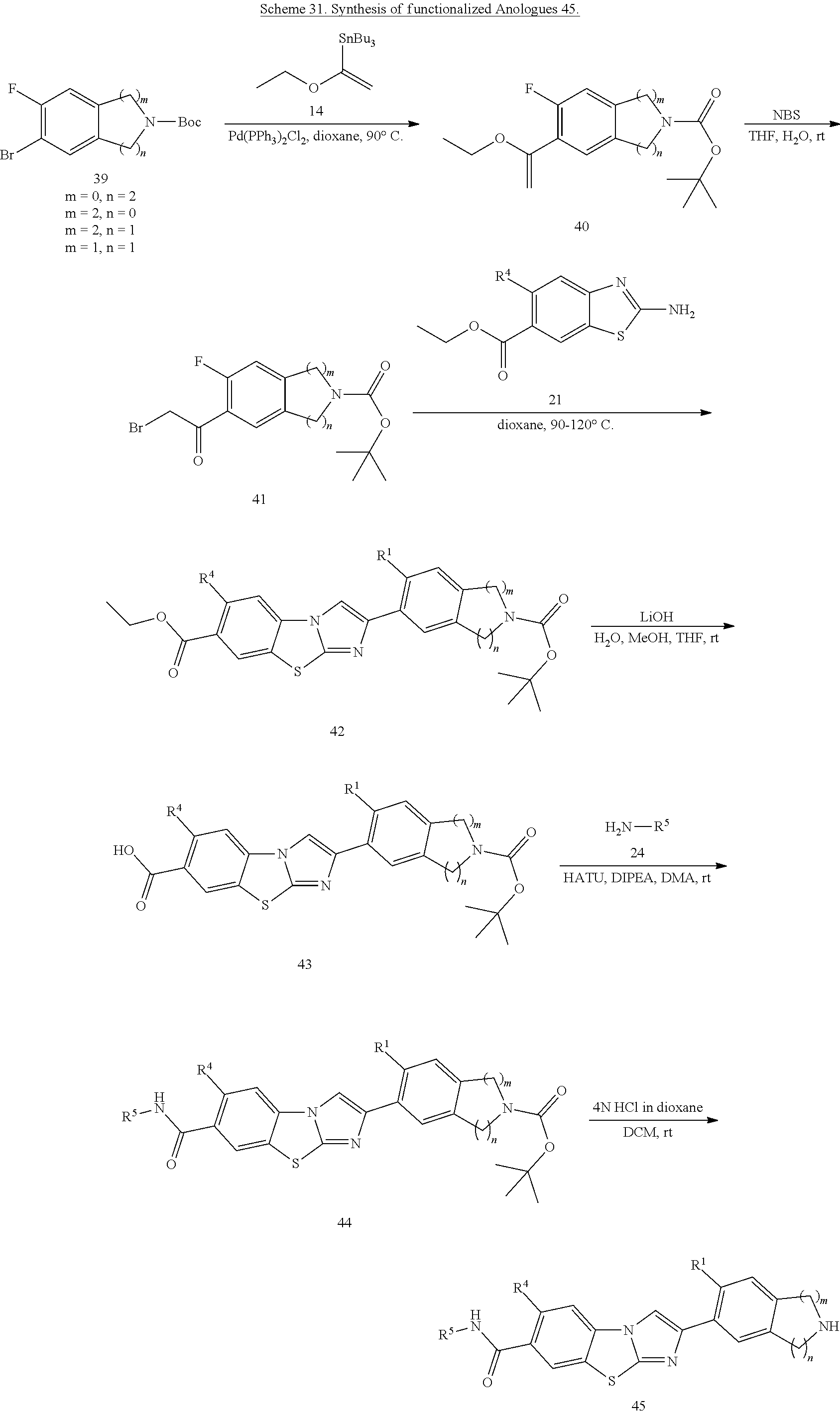

The first 2 steps are analogous to corresponding steps 4 and 5 as described in Scheme 24 to generate modified a-bromoketone intermediates 41. The subsequent 4 steps were also undertaken as described in Scheme 27 affording the final compounds 45 (Scheme 31).

### Example 3

### Synthetic Details for Various Intermediates of Compounds of the Invention (Schemes 32-75)

### Tert-butyl methyl(2,2,2-trifluoro-1-(4-(4,4,5,5-tetramethyl-1,3,2-dioxaborolan-2-yl)phenyl)ethyl)carbamate (Intermediate A, Scheme 32)

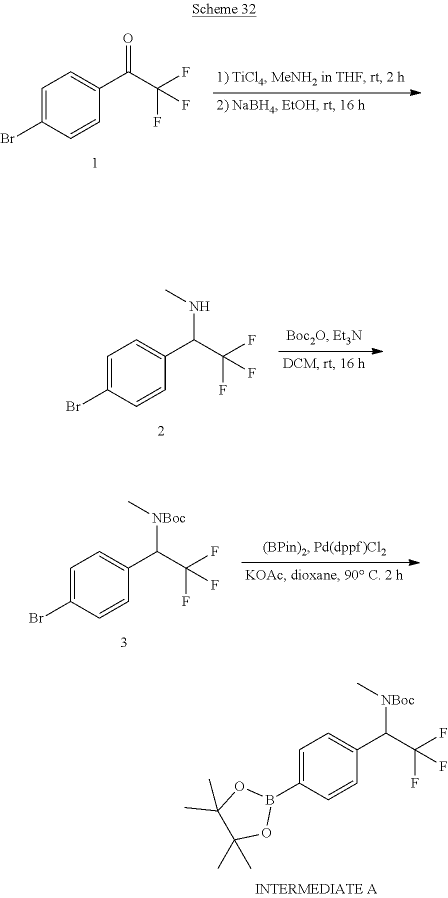 [SMILES: O=C(c1ccc(Br)cc1)C(F)(F)F ; CNC(c1ccc(Br)cc1)C(F)(F)F ; CN(C(=O)OC(C)(C)C)C(c1ccc(Br)cc1)C(F)(F)F ; CN(C(=O)OC(C)(C)C)C(c1ccc(B2OC(C)(C)C(C)(C)O2)cc1)C(F)(F)F]

### Step 1: 1-(4-Bromophenyl)-2,2,2-trifluoro-N-methylethan-1-amine

To a stirred solution of 1-(4-bromophenyl)-2,2,2-trifluoroethan-1-one (2.50 g, 9.88 mmol) in THF (25 mL) were added titanium tetrachloride (11.24 g, 59.26 mmol) and a 2M solution of methylamine in THF (19 mL, 38.00 mmol) at room temperature under a nitrogen atmosphere. The resulting mixture was stirred for additional 2 h at room temperature. The resulting solution was diluted with hexane (500 mL). The precipitated solids were filtered out. The filtrate was concentrated under reduced pressure. The residue was taken up with ethanol (25 mL) followed by the addition of sodium borohydride (0.75 g, 19.83 mmol). The resulting mixture was stirred for additional 16 h at room temperature. The reaction was quenched with water (100 mL) and extracted with ethyl acetate (3×150 mL). The combined organic layers were washed with brine (200 mL) and dried over anhydrous Na₂SO₄. After filtration, the filtrate was concentrated under reduced pressure to afford 1-(4-bromophenyl)-2,2,2-trifluoro-N-methylethan-1-amine as a colorless oil.

Yield: 1.30 g (49%). ¹H NMR (400 MHz, CDCl₃) δ 7.56 (d, J=8.4 Hz, 2H), 7.31 (d, J=8.4 Hz, 2H), 4.03 (q, J=7.2 Hz, 1H), 2.41 (d, J=0.8 Hz, 3H), 1.86 (br s, 1H). m/z: [ESI⁺] 268, 270 (M+H)⁺.

### Step 2: Tert-butyl (1-(4-bromophenyl)-2,2,2-trifluoroethyl)(methyl)carbamate

To a stirred solution of 1-(4-bromophenyl)-2,2,2-trifluoro-N-methylethan-1-amine (1.00 g, 3.73 mmol) in DCM (10 mL) were added triethylamine (0.75 g, 7.41 mmol) and di-tert-butyl dicarbonate (1.63 g, 7.47 mmol). The resulting mixture was stirred for 16 h at room temperature. The resulting mixture was diluted with water (100 mL) and extracted with DCM (3×100 mL). The combined organic layers were concentrated under reduced pressure. The residue was purified by silica gel column chromatography, eluted with 10% of ethyl acetate in petroleum ether. The fractions containing desired product were collected and concentrated under reduced pressure to afford tert-butyl (1-(4-bromophenyl)-2,2,2-trifluoroethyl)(methyl)carbamate as a brown solid.

Yield: 1.30 g (95%). ¹H NMR (400 MHz, CDCl₃) δ 7.59 (d, J=8.6 Hz, 2H), 7.33 (d, J=8.6 Hz, 2H), 6.06 (q, J=8.4 Hz, 1H), 2.82 (t, J=1.2 Hz, 3H), 1.58 (s, 9H). m/z: [ESI⁺] 312, 314 (M+H−56)⁺.

### Step 3: Tert-butyl methyl(2,2,2-trifluoro-1-(4-(4,4,5,5-tetramethyl-1,3,2-dioxaborolan-2-yl)phenyl)ethyl)carbamate

To a stirred solution of tert-butyl (1-(4-bromophenyl)-2,2,2-trifluoroethyl)(methyl)carbamate (1.30 g, 3.53 mmol) in dioxane (15 mL) were added bis(pinacolato)diboron (1.34 g, 5.28 mmol), KOAc (1.04 g, 10.60 mmol) and [1,1′-Bis(diphenylphosphino)ferrocene]dichloropalladium(II) (0.26 g, 0.36 mmol) at room temperature under an argon atmosphere. The resulting mixture was stirred for additional 2 h at 90° C. The resulting mixture was cooled down to room temperature and diluted with water (100 mL). The resulting mixture was extracted with ethyl acetate (3×100 mL). The combined organic layers were concentrated under reduced pressure. The residue was purified by reverse phase flash chromatography with the following conditions: Column: Spherical C18, 20-40 μm, 330 g; Mobile Phase A: water (plus 10 mM formic acid); Mobile Phase B: ACN; Flow rate: 80 mL/min; Gradient: 10% B-30% B in 20 min; Detector: UV 220/254 nm. The fractions containing desired product were collected and concentrated under reduced pressure to afford tert-butyl methyl(2,2,2-trifluoro-1-(4-(4,4,5,5-tetramethyl-1,3,2-dioxaborolan-2-yl)phenyl)ethyl)carbamate as an off-white solid.

Yield: 1.40 g (95%). ¹H NMR (400 MHz, CDCl₃) δ 7.98 (d, J=8.0 Hz, 2H), 7.55 (d, J=8.0 Hz, 2H), 4.81 (q, J=6.8 Hz, 1H), 2.72 (s, 2H), 1.39 (s, 9H), 1.31 (s, 12H). m/z: [ESI⁺] 360 (M+H−56)⁺.

### 6-(5-Hydroxy-3-(p-tolyl)-1H-pyrazol-1-yl)nicotinic acid (Intermediate B)

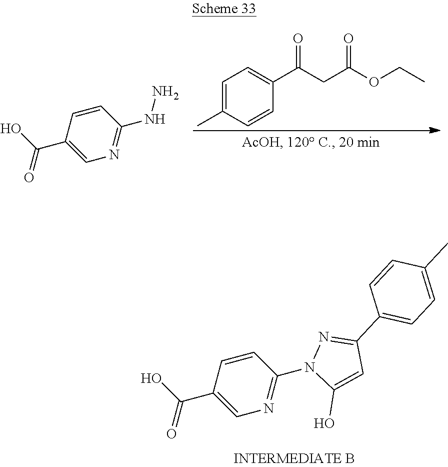 [SMILES: NNc1ccc(C(=O)O)cn1 ; CCOC(=O)CC(=O)c1ccc(C)cc1 ; Cc1ccc(-c2cc(O)n(-c3ccc(C(=O)O)cn3)n2)cc1]

To a solution of 6-hydrazineylnicotinic acid (0.37 g, 2.42 mmol) in AcOH (10 mL) was added ethyl 3-oxo-3-(p-tolyl)propanoate (0.50 g, 2.42 mmol) at room temperature. The resulting mixture was stirred for 20 min at 120° C. Upon completion, the resulting mixture was cooled down to room temperature and concentrated under reduced pressure. The residue was triturated with DCM (3×30 mL) and dried in the air to afford 6-(5-hydroxy-3-(p-tolyl)-1H-pyrazol-1-yl)nicotinic acid as a light yellow solid.

Yield: 0.50 g (70%). ¹H NMR (400 MHz, DMSO) δ 8.89 (d, J=1.8 Hz, 1H), 8.33 (dd, J=1.8, 8.6 Hz, 1H), 8.10 (d, J=8.6 Hz, 1H), 7.72 (d, J=8.0 Hz, 2H), 7.23 (d, J=8.0 Hz, 2H), 5.80 (s, 1H), 2.34 (s, 3H). m/z: [ESI⁺] 296 (M+H)⁺.

### 2-Bromobenzo[d]imidazo[2,1-b]thiazole-7-carboxylic acid (Intermediate C)

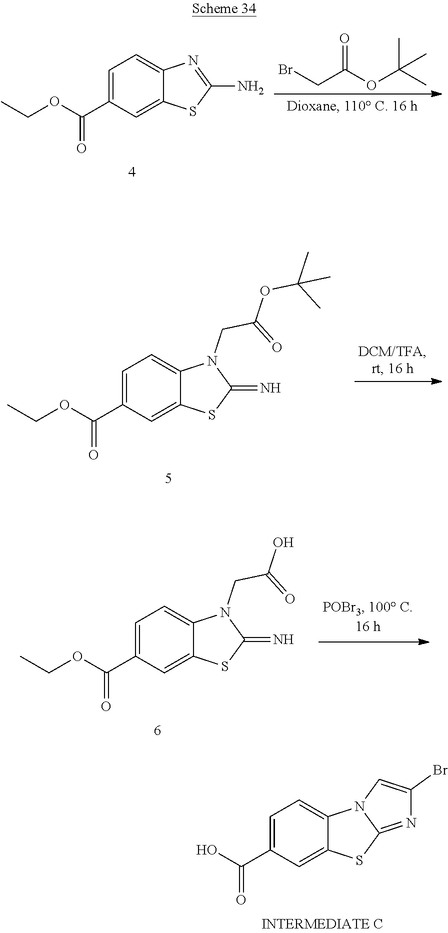

### Step 1: Ethyl 3-(2-(tert-butoxy)-2-oxoethyl)-2-imino-2,3-dihydrobenzo[d]thiazole-6-carboxylate

To a stirred solution of ethyl 2-aminobenzo[d]thiazole-6-carboxylate (75.00 g, 0.337 mol) in dioxane (800 mL) was added tert-butyl 2-bromoacetate (78.98 g, 0.405 mol) at room temperature under a nitrogen atmosphere. The resulting mixture was stirred at 110° C. for 16 h under a nitrogen atmosphere. Upon completion, the resulting mixture was cooled down to room temperature. The precipitated solids were collected by filtration, washed with ethanol (3×120 mL) and dried in the air to afford ethyl 3-(2-(tert-butoxy)-2-oxoethyl)-2-imino-2,3-dihydrobenzo[d]thiazole-6-carboxylate as an off-white solid.

Yield: 97.50 g (86%). ¹H NMR (400 MHz, DMSO) δ 10.68 (br s, 1H), 8.68 (d, J=1.8 Hz, 1H), 8.11 (dd, J=1.8, 8.6 Hz, 1H), 7.77 (d, J=8.6 Hz, 1H), 5.24 (s, 2H), 4.36 (q, J=7.2 Hz, 2H), 1.44 (s, 9H), 1.35 (t, J=7.2 Hz, 3H). m/z: [ESI⁺] 337 (M+H)⁺.

### Step 2: 2-(6-(Ethoxycarbonyl)-2-iminobenzo[d]thiazol-3(2H)-yl)acetic acid

To a solution of ethyl 3-(2-(tert-butoxy)-2-oxoethyl)-2-imino-2,3-dihydrobenzo[d]thiazole-6-carboxylate (97.00 g, 0.288 mol) in DCM (600 mL) was added trifluoroacetic acid (300 mL). The resulting solution was stirred for 16 h at room temperature. The resulting mixture was concentrated under reduced pressure. The residue was triturated with diethyl ether (400 mL) and dried in the air to afford 2-(6-(ethoxycarbonyl)-2-iminobenzo[d]thiazol-3(2H)-yl)acetic acid as an off-white solid.

Yield 80.00 g (99%). ¹H NMR (400 MHz, DMSO) δ 13.81 (br s, 1H), 10.72 (br s, 1H), 8.69 (d, J=1.8 Hz, 1H), 8.10 (dd, J=1.8, 8.6 Hz, 1H), 7.82 (d, J=8.6 Hz, 1H), 5.22 (s, 2H), 4.36 (q, J=7.2 Hz, 2H), 1.35 (t, J=7.2 Hz, 3H). m/z: [ESI⁺] 281 (M+H)⁺.

### Step 3: 2-Bromobenzo[d]imidazo[2,1-b]thiazole-7-carboxylic acid

A mixture of 2-(6-(ethoxycarbonyl)-2-iminobenzo[d]thiazol-3(2H)-yl)acetic acid (80.00 g, 0.285 mol) and phosphorylbromide (654.58 g, 2.283 mol) was stirred for 16 h at 100° C. under a nitrogen atmosphere. Upon completion, the resulting mixture was cooled down to room temperature and diluted with dioxane (600 mL). The precipitated solids were collected by filtration, washed with water (6×180 mL) and dried in the air to afford 2-bromobenzo[d]imidazo[2,1-b]thiazole-7-carboxylic acid as an off-white solid.

Yield 62.80 g (74%). ¹H NMR (400 MHz, DMSO) δ 8.70 (d, J=1.6 Hz, 1H), 8.59 (s, 1H), 8.12 (dd, J=1.6, 8.4 Hz, 1H), 8.10 (d, J=8.4 Hz, 1H). m/z: [ESI⁺] 297, 299 (M+H)⁺.

### 2-(2-Fluoro-3-methylphenyl)benzo[d]imidazo[2,1-b]thiazole-7-carboxylic acid (Intermediate D)

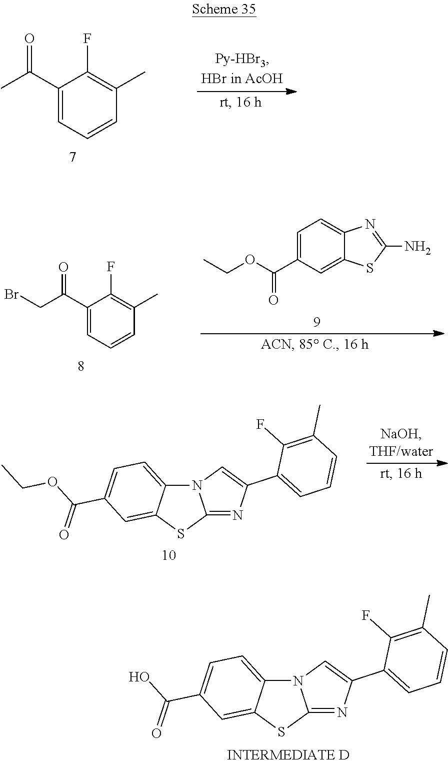 [SMILES: CC(=O)c1cccc(C)c1F ; Cc1cccc(C(=O)CBr)c1F ; CCOC(=O)c1ccc2nc(N)sc2c1 ; CCOC(=O)c1ccc2c(c1)sc1nc(-c3cccc(C)c3F)cn12 ; Cc1cccc(-c2cn3c(n2)sc2cc(C(=O)O)ccc23)c1F]

### Step 1: 2-Bromo-1-(2-fluoro-3-methylphenyl)ethan-1-one

To a stirred solution of 1-(2-fluoro-3-methylphenyl)ethan-1-one (3.50 g, 23.00 mmol) in a solution of HBr in AcOH (40 mL, containing 33% HBr, w/w) was added pyridinium bromide-perbromide (7.36 g, 23.01 mmol). The resulting mixture was stirred for 3 h at room temperature under a nitrogen atmosphere. The reaction was quenched by the addition of water (200 mL) at room temperature. The resulting mixture was extracted with ethyl acetate (3×50 mL). The combined organic layers were dried over anhydrous Na₂SO₄. After filtration, the filtrate was concentrated under reduced pressure to afford 2-bromo-1-(2-fluoro-3-methylphenyl)ethan-1-one as a yellow oil.

Yield 4.90 g (92%). ¹H NMR (400 MHz, CDCl₃) δ 7.76-7.74 (m, 1H), 7.46-7.44 (m, 1H), 7.16-7.14 (m, 1H), 4.55 (d, J=2.5 Hz, 2H), 2.12 (s, 3H).

### Step 2: ethyl 2-(2-fluoro-3-methylphenyl)benzo[d]imidazo[2,1-b]thiazole-7-carboxylate

To a stirred solution of 2-bromo-1-(2-fluoro-3-methylphenyl)ethan-1-one (1.50 g, 6.49 mmol) in acetonitrile (20 mL) was added ethyl 2-aminobenzo[d]thiazole-6-carboxylate (1.44 g, 6.49 mmol) at room temperature under a nitrogen atmosphere. The resulting mixture was stirred for 16 h at 85° C. under a nitrogen atmosphere. Upon completion, the resulting mixture was cooled down to room temperature. The precipitated solids were collected by filtration, washed with water (3×10 mL) and dried in the air to afford ethyl 2-(2-fluoro-3-methylphenyl)benzo[d]imidazo[2,1-b]thiazole-7-carboxylate as a brown solid.

Yield 1.17 g (51%). ¹H NMR (400 MHz, DMSO) δ 8.74 (d, J=3.8 Hz, 1H), 8.69 (d, J=1.6 Hz, 1H), 8.28 (d, J=8.4 Hz, 1H), 8.11 (dd, J=1.6, 8.4 Hz, 1H), 7.97-7.92 (m, 1H), 7.25-7.16 (m, 2H), 4.36 (q, J=7.2 Hz, 2H), 2.33 (d, J=2.4 Hz, 3H), 1.36 (t, J=7.2 Hz, 3H). m/z: [ESI⁺] 355 (M+H)⁺.

### Analytical Data for Intermediates Synthesized According to the Methods Described Above

The following compounds below were synthesized according to the described procedure above.

### Ethyl 2-(m-tolyl)benzo[d]imidazo[2,1-b]thiazole-7-carboxylate

Starting from 2-bromo-1-(m-tolyl)ethan-1-one (60.00 g, 281.60 mmol). Yield 20.00 g (21%), as an off-white solid. ¹H NMR (400 MHz, DMSO) δ 8.89 (s, 1H), 8.71 (d, J=1.6 Hz, 1H), 8.15 (dd, J=1.6, 8.4, Hz, 1H), 8.10 (d, J=8.4 Hz, 1H), 7.70 (d, J=1.8 Hz, 1H), 7.66 (d, J=7.6 Hz, 1H), 7.34 (dd, J=1.6, 7.6 Hz, 1H), 7.14 (d, J=7.6 Hz, 1H), 4.36 (q, J=7.2 Hz, 2H), 2.37 (s, 3H), 1.36 (t, J=7.2 Hz, 3H). m/z: [ESI⁺] 337 (M+H)⁺.

### Methyl 6-(p-tolyl)imidazo[2,1-b]thiazole-2-carboxylate

Starting from 2-bromo-1-(p-tolyl)ethan-1-one (2.69 g, 12.62 mmol). Yield 1.40 g (41%), as a white solid. ¹H NMR (400 MHz, DMSO) δ 8.79 (s, 1H), 8.22 (s, 1H), 7.76 (d, J=8.0 Hz, 2H), 7.23 (d, J=8.0 Hz, 2H), 3.89 (s, 3H), 2.33 (s, 3H). m/z: [ESI⁺] 273 (M+H)⁺.

### Ethyl 2-(4-bromophenyl)benzo[d]imidazo[2,1-b]thiazole-7-carboxylate

Starting from 2-bromo-1-(4-bromophenyl)ethanone (13.76 g, 49.51 mmol). Yield 7.80 g (39%), as an off-white solid. ¹H NMR (400 MHz, DMSO) δ 8.90 (s, 1H), 8.68 (s, 1H), 8.12 (d, J=8.4 Hz, 1H), 8.05 (d, J=8.4 Hz, 1H), 7.80 (d, J=8.2 Hz, 2H), 7.63 (d, J=8.2 Hz, 2H), 4.36 (q, J=7.2 Hz, 2H), 1.36 (t, J=7.2 Hz, 3H). m/z: [ESI⁺] 401, 403 (M+H)⁺.

### Ethyl 2-(3-bromophenyl)benzo[d]imidazo[2,1-b]thiazole-7-carboxylate

Starting from 2-bromo-1-(3-bromophenyl)ethanone (10.00 g, 35.98 mmol). Yield 8.45 g (59%), as a white solid. ¹H NMR (400 MHz, DMSO) δ 8.98 (s, 1H), 8.71 (d, J=1.6 Hz, 1H), 8.16 (dd, J=1.8, 8.4 Hz, 1H), 8.06 (s, 1H), 8.05 (d, J=8.4 Hz, 1H), 7.87 (d, J=7.8 Hz, 1H), 7.50 (d, J=8.4 Hz, 1H), 7.42 (dd, J=1.6, 7.8 Hz, 1H), 4.37 (q, J=7.2 Hz, 2H), 1.36 (t, J=7.2 Hz, 3H). m/z: [ESI⁺]401, 403 (M+H)⁺.

### Ethyl 2-(3-cyanophenyl)benzo[d]imidazo[2,1-b]thiazole-7-carboxylate

Starting from 3-(2-bromoacetyl)benzonitrile (1.00 g, 4.46 mmol). Yield 0.45 g (29%), as a white solid. ¹H NMR (400 MHz, DMSO) δ 9.03 (s, 1H), 8.73 (d, J=1.6 Hz, 1H), 8.27 (dd, J=1.6, 2.0 Hz, 1H), 8.20 (d, J=8.4 Hz, 1H), 8.17 (dd, J=1.6, 8.4 Hz, 1H), 8.04 (d, J=8.4 Hz, 1H), 7.77 (dd, J=1.6, 7.8 Hz, 1H), 7.67 (dd, J=1.6, 7.8 Hz, 1H), 4.37 (q, J=7.2 Hz, 2H), 1.36 (t, J=7.2 Hz, 3H). m/z: [ESI⁺] 348 (M+H)⁺.

### Ethyl 2-(2-fluoro-5-methylphenyl)benzo[d]imidazo[2,1-b]thiazole-7-carboxylate

Starting from 2-bromo-1-(2-fluoro-5-methylphenyl)ethan-1-one (1.00 g, 4.33 mmol). Yield 0.60 g (39%), as a brown solid. ¹H NMR (400 MHz, DMSO) δ 8.70 (s, 1H), 8.69 (d, J=1.6 Hz, 1H), 8.28 (d, J=2.4 Hz, 1H), 8.10 (dd, J=1.6, 8.4 Hz, 1H), 7.96 (d, J=8.4 Hz, 1H), 7.21 (dd, J=8.4, 11.2 Hz, 1H), 7.15 (dd, J=2.4, 8.4 Hz, 1H), 4.36 (q, J=7.2 Hz, 2H), 2.36 (s, 3H), 1.36 (t, J=7.2 Hz, 3H). m/z: [ESI⁺] 355 (M+H)⁺.

### 2-Phenylbenzo[d]imidazo[2,1-b]thiazole-7-carboxylic acid

Starting from 2-bromo-1-phenylethan-1-one (2.25 g, 11.33 mmol). Yield 0.75 g (23%), as a white solid. ¹H NMR (400 MHz, DMSO) δ 8.96 (s, 1H), 8.84 (d, J=1.6 Hz, 1H), 8.40 (dd, J=1.6, 8.6 Hz, 1H), 8.30 (d, J=8.6 Hz, 1H), 7.86 (dd, J=1.8, 7.2 Hz, 2H), 7.64-7.49 (m, 3H). m/z: [ESI⁺] 295 (M+H)⁺.

### 2-(4-Chlorophenyl)benzo[d]imidazo[2,1-b]thiazole-7-carboxylic acid

Starting from 2-bromo-1-(4-chlorophenyl)ethan-1-one (397 mg, 1.700 mmol). Yield 160 mg (28%), as an off-white solid. ¹H NMR (400 MHz, DMSO) δ 13.13 (br s, 1H), 8.88 (s, 1H), 8.67 (d, J=1.6 Hz, 1H), 8.14 (dd, J=1.6, 8.4 Hz, 1H), 8.05 (d, J=8.4 Hz, 1H), 7.89 (d, J=8.6 Hz, 2H), 7.51 (d, J=8.6 Hz, 2H). m/z: [ESI⁺] 329, 331 (M+H)⁺.

### 2-(3-Methoxyphenyl)benzo[d]imidazo[2,1-b]thiazole-7-carboxylic acid

Starting from 2-bromo-1-(3-methoxyphenyl)ethan-1-one (2.59 g, 11.31 mmol). Yield 0.60 g (16%), as a white solid. ¹H NMR (400 MHz, DMSO) δ 13.19 (br s, 1H), 8.87 (s, 1H), 8.67 (d, J=1.6 Hz, 1H), 8.14 (dd, J=1.6, 8.4 Hz, 1H), 8.05 (d, J=8.4 Hz, 1H), 7.48-7.46 (m, 1H), 7.46 (d, J=1.8 Hz, 1H), 7.36 (dd, J=1.6, 8.0 Hz, 1H), 6.92-6.85 (m, 1H), 3.83 (s, 3H). m/z: [ESI⁺] 325 (M+H)⁺.

### 4-(7-(Ethoxycarbonyl)benzo[d]imidazo[2,1-b]thiazol-2-yl)benzoic acid

Starting from 4-(2-bromoacetyl)benzoic acid (10.00 g, 41.14 mmol). Yield 13.00 g (86%), as a white solid. ¹H NMR (400 MHz, DMSO) δ 9.02 (s, 1H), 8.71 (d, J=1.6 Hz, 1H), 8.15 (dd, J=1.6, 8.4 Hz, 1H), 8.10 (d, J=8.4 Hz, 1H), 8.02 (d, J=8.8 Hz, 2H), 7.98 (d, J=8.8 Hz, 2H), 4.35 (q, J=7.2 Hz, 2H), 1.36 (t, J=7.2 Hz, 3H). m/z: [ESI⁺] 367 (M+H)⁺.

### 7-Bromo-2-(o-tolyl)benzo[d]imidazo[2,1-b]thiazole

Starting from 6-bromobenzo[d]thiazol-2-amine (2.00 g, 8.73 mmol). Yield 1.60 g (53%), as a white solid. ¹H NMR (400 MHz, DMSO) δ 8.65 (s, 1H), 8.40 (d, J=1.8 Hz, 1H), 8.14 (d, J=8.6 Hz, 1H), 7.87-7.78 (m, 2H), 7.36-7.23 (m, 3H), 2.54 (s, 3H). m/z: [ESI⁺] 343, 345 (M+H)⁺.

### 7-Bromo-2-(4-isopropylphenyl)benzo[d]imidazo[2,1-b]thiazole

Starting from 6-bromobenzo[d]thiazol-2-amine (1.00 g, 4.36 mmol). Yield 1.20 g (74%), as a white solid. ¹H NMR (400 MHz, DMSO) δ 8.80 (s, 1H), 8.38 (d, J=2.0 Hz, 1H), 7.99 (d, J=8.6 Hz, 1H), 7.79 (dd, J=2.0, 8.6 Hz, 1H), 7.77 (d, J=8.4 Hz, 2H), 7.33 (d, J=8.4 Hz, 2H), 2.93-2.90 (m, 1H), 1.24 (d, J=7.0 Hz, 6H). m/z: [ESI⁺] 371, 373 (M+H)⁺.

### 7-Bromo-2-(2-fluorophenyl)benzo[d]imidazo[2,1-b]thiazole

Starting from 2-bromo-1-(2-fluorophenyl)ethan-1-one (1.00 g, 4.61 mmol). Yield 1.20 g (75%), as a white solid. ¹H NMR (400 MHz, DMSO) δ 8.72 (s, 1H), 8.36 (d, J=1.8 Hz, 1H), 8.18 (d, J=8.6 Hz, 1H), 8.14 (d, J=7.8 Hz, 1H), 7.76 (dd, J=2.0, 8.6 Hz, 1H), 7.42-7.27 (m, 3H). m/z: [ESI⁺]347, 349 (M+H)⁺.

### 7-Bromo-2-(3-fluorophenyl)benzo[d]imidazo[2,1-b]thiazole

Starting from 2-bromo-1-(3-fluorophenyl)ethan-1-one (1.00 g, 4.61 mmol). Yield 1.00 g (62%), as a white solid. ¹H NMR (400 MHz, DMSO) δ 8.85 (s, 1H), 8.37 (d, J=2.0 Hz, 1H), 7.94 (dd, J=1.6, 8.6 Hz, 1H), 7.78 (dd, J=2.0, 8.6 Hz, 1H), 7.70 (d, J=7.8 Hz, 1H), 7.63 (d, J=10.4 Hz, 1H), 7.49 (dd, J=6.2, 8.0 Hz, 1H), 7.13 (dd, J=2.6, 8.4 Hz, 1H). m/z: [ESI⁺] 347, 349 (M+H)⁺.

### 7-Bromo-2-(3-chlorophenyl)benzo[d]imidazo[2,1-b]thiazole

Starting from 6-bromobenzo[d]thiazol-2-amine (1.00 g, 4.36 mmol). Yield 1.10 g (69%), as a white solid. ¹H NMR (400 MHz, DMSO) δ 8.91 (s, 1H), 8.37 (d, J=2.0 Hz, 1H), 7.92 (d, J=8.6 Hz, 1H), 7.90 (dd, J=2.0, 2.4 Hz, 1H), 7.82 (dd, J=1.2, 7.8 Hz, 1H), 7.80 (dd, J=2.0, 8.0 Hz, 1H), 7.48 (dd, J=2.0, 8.0 Hz, 1H), 7.36 (dd, J=2.0, 8.0 Hz, 1H). m/z: [ESI⁺] 363, 365, 367 (M+H)⁺.

### 7-Bromo-2-(4-chlorophenyl)benzo[d]imidazo[2,1-b]thiazole

Starting from 6-bromobenzo[d]thiazol-2-amine (1.00 g, 4.36 mmol). Yield 1.20 g (76%), as a white solid. ¹H NMR (400 MHz, DMSO) δ 8.84 (s, 1H), 8.37 (d, J=2.0 Hz, 1H), 7.94 (d, J=8.6 Hz, 1H), 7.88 (d, J=8.6 Hz, 2H), 7.78 (dd, J=2.0, 8.6 Hz, 1H), 7.51 (d, J=8.6 Hz, 2H). m/z: [ESI⁺] 363, 365, 367 (M+H)⁺.

### 7-Bromo-2-(2-chlorophenyl)benzo[d]imidazo[2,1-b]thiazole

Starting from 6-bromobenzo[d]thiazol-2-amine (1.00 g, 4.36 mmol). Yield 0.70 g (44%), as a white solid. ¹H NMR (400 MHz, DMSO) δ 8.97 (s, 1H), 8.37 (s, 1H), 8.22-8.16 (m, 2H), 7.81-7.74 (m, 1H), 7.56 (d, J=8.0 Hz, 1H), 7.45 (dd, J=1.6, 7.6 Hz, 1H), 7.35 (dd, J=1.6, 7.6 Hz, 1H). m/z: [ESI⁺] 363, 365, 367 (M+H)⁺.

### 7-Bromo-2-(4-ethylphenyl)benzo[d]imidazo[2,1-b]thiazole

Starting from 2-bromo-1-(4-ethylphenyl)ethan-1-one (1.00 g, 4.40 mmol). Yield 0.80 g (51%), as a white solid. ¹H NMR (400 MHz, DMSO) δ 8.80 (s, 1H), 8.38 (d, J=2.0 Hz, 1H), 7.98 (dd, J=1.4, 8.6 Hz, 1H), 7.82-7.74 (m, 3H), 7.31 (d, J=8.0 Hz, 2H), 2.64 (q, J=7.6 Hz, 2H), 1.21 (t, J=7.6 Hz, 3H). m/z: [ESI⁺] 357, 359 (M+H)⁺.

### 7-Bromo-2-(m-tolyl)benzo[d]imidazo[2,1-b]thiazole

Starting from 2-bromo-1-(m-tolyl)ethan-1-one (1.00 g, 4.69 mmol). Yield 0.62 g (39%), as an off-white solid. ¹H NMR (400 MHz, DMSO) δ 8.81 (s, 1H), 8.37 (d, J=2.0 Hz, 1H), 7.97 (d, J=8.6 Hz, 1H), 7.78 (dd, J=2.0, 8.6 Hz, 1H), 7.69 (d, J=1.8 Hz, 1H), 7.65 (d, J=7.8 Hz, 1H), 7.34 (dd, J=1.6, 7.6 Hz, 1H), 7.13 (d, J=7.6 Hz, 1H), 2.37 (s, 3H). m/z: [ESI⁺] 343, 345 (M+H)⁺.

### 4-(7-Bromobenzo[d]imidazo[2,1-b]thiazol-2-yl)-N-methylbenzamide

Starting from 4-(2-bromoacetyl)-N-methylbenzamide (1.00 g, 3.90 mmol). Yield 0.70 g (46%), as a white solid. ¹H NMR (400 MHz, DMSO) δ 8.91 (s, 1H), 8.44 (q, J=4.2 Hz, 1H), 8.37 (d, J=2.0 Hz, 1H), 8.00-7.87 (m, 5H), 7.79 (dd, J=2.0, 8.6 Hz, 1H), 2.81 (d, J=4.2 Hz, 3H). m/z: [ESI⁺]386, 388 (M+H)⁺.

### 7-Bromo-2-(4-methoxyphenyl)benzo[d]imidazo[2,1-b]thiazole

Starting from 6-bromobenzo[d]thiazol-2-amine (1.00 g, 4.36 mmol). Yield 0.80 g (51%), as a brown solid. ¹H NMR (400 MHz, DMSO) δ 8.86 (s, 1H), 8.43 (d, J=2.0 Hz, 1H), 8.03 (d, J=8.6 Hz, 1H), 7.82 (dd, J=2.0, 8.6 Hz, 1H), 7.76 (d, J=8.8 Hz, 2H), 7.05 (d, J=8.8 Hz, 2H), 3.80 (s, 3H). m/z: [ESI⁺] 359, 361 (M+H)⁺.

### Step 3: 2-(2-Fluoro-3-methylphenyl)benzo[d]imidazo[2,1-b]thiazole-7-carboxylic acid

To a stirred solution of ethyl 2-(2-fluoro-3-methylphenyl)benzo[d]imidazo[2,1-b]thiazole-7-carboxylate (0.50 g, 1.41 mmol) in THF (5 mL) were added water (5 mL) and NaOH (0.28 g, 7.00 mmol) at room temperature under a nitrogen atmosphere. The resulting mixture was stirred for 16 at room temperature under a nitrogen atmosphere. The resulting mixture was acidified to pH 5 with 2N HCl (4 mL). The precipitated solids were collected by filtration and dried in the air to afford 2-(2-fluoro-3-methylphenyl)benzo[d]imidazo[2,1-b]thiazole-7-carboxylic acid as a white solid.

Yield: 0.20 g (43%). ¹H NMR (400 MHz, DMSO) δ 13.14 (br s, 1H), 8.75 (d, J=4.0 Hz, 1H), 8.67 (d, J=1.6 Hz, 1H), 8.28 (d, J=8.4 Hz, 1H), 8.11 (dd, J=1.6, 8.4 Hz, 1H), 7.98 (dd, J=2.0, 7.4 Hz, 1H), 7.28-7.17 (m, 2H), 2.34 (d, J=2.0 Hz, 3H). m/z: [ESI⁺] 327 (M+H)⁺.

### Analytical Data for Intermediates Synthesized According to the Methods Described Above

The following compounds below were synthesized according to the described procedure above, using the corresponding ester as Starting material.

### 2-(m-Tolyl)benzo[d]imidazo[2,1-b]thiazole-7-carboxylic acid

Starting from ethyl 2-(m-tolyl)benzo[d]imidazo[2,1-b]thiazole-7-carboxylate (1.77 g, 5.26 mmol). Yield 1.35 g (83%), as an off-white solid. ¹H NMR (400 MHz, DMSO) δ 8.83 (s, 1H), 8.66 (d, J=1.6 Hz, 1H), 8.13 (dd, J=1.6, 8.4 Hz, 1H), 8.06 (d, J=8.4 Hz, 1H), 7.72 (d, J=2.2 Hz, 1H), 7.67 (d, J=7.6 Hz, 1H), 7.33 (dd, J=1.6, 7.6 Hz, 1H), 7.13 (d, J=7.6 Hz, 1H), 2.37 (s, 3H). m/z: [ESI⁺]309 (M+H)⁺.

### 6-(p-Tolyl)imidazo[2,1-b]thiazole-2-carboxylic acid

Starting from ethyl 6-(p-tolyl)imidazo[2,1-b]thiazole-2-carboxylate (3.50 g, 12.22 mmol). Yield 3.00 g (95%), as a white solid. ¹H NMR (400 MHz, DMSO) δ 8.67 (s, 1H), 8.20 (s, 1H), 7.75 (d, J=8.0 Hz, 2H), 7.23 (d, J=8.0 Hz, 2H), 2.33 (s, 3H). m/z: [ESI⁺] 259 (M+H)⁺.

### 2-(4-Bromophenyl)benzo[d]imidazo[2,1-b]thiazole-7-carboxylic acid

Starting from ethyl 2-(4-bromophenyl)benzo[d]imidazo[2,1-b]thiazole-7-carboxylate (7.80 g, 19.44 mmol). Yield 5.80 g (80%), as an off-white solid. ¹H NMR (400 MHz, DMSO) δ 13.19 (br s, 1H), 8.90 (s, 1H), 8.69 (d, J=1.6 Hz, 1H), 8.14 (dd, J=1.6, 8.4 Hz, 1H), 8.04 (d, J=8.4 Hz, 1H), 7.83 (d, J=8.6 Hz, 2H), 7.65 (d, J=8.6 Hz, 2H). m/z: [ESI⁺] 373, 375 (M+H)⁺.

### 2-(3-Bromophenyl)benzo[d]imidazo[2,1-b]thiazole-7-carboxylic acid

Starting from ethyl 2-(3-bromophenyl)benzo[d]imidazo[2,1-b]thiazole-7-carboxylate (8.45 g, 21.06 mmol). Yield 7.00 g (89%), as a yellow solid. ¹H NMR (400 MHz, DMSO) δ 13.16 (br s, 1H), 8.97 (s, 1H), 8.70 (d, J=1.8 Hz, 1H), 8.15 (dd, J=1.8, 8.4 Hz, 1H), 8.08-7.99 (m, 2H), 7.88 (d, J=7.8 Hz, 1H), 7.50 (d, J=7.8 Hz, 1H), 7.42 (dd, J=1.6, 7.8 Hz, 1H). m/z: [ESI⁺] 373, 375 (M+H)⁺.

### 2-(3-Bromophenyl)benzo[d]imidazo[2,1-b]thiazole-7-carboxylic acid

Starting from ethyl 2-(3-cyanophenyl)benzo[d]imidazo[2,1-b]thiazole-7-carboxylate (0.45 g, 1.30 mmol). Yield 0.40 g (96%), as a white solid. ¹H NMR (400 MHz, DMSO) δ 9.01 (s, 1H), 8.69 (s, 1H), 8.26 (s, 1H), 8.20-8.15 (m, 2H), 8.02 (s, 1H), 7.76 (s, 1H), 7.66 (s, 1H). m/z: [ESI⁺] 320 (M+H)⁺.

### 2-(2-Fluoro-5-methylphenyl)benzo[d]imidazo[2,1-b]thiazole-7-carboxylic acid

Starting from ethyl 2-(2-fluoro-5-methylphenyl)benzo[d]imidazo[2,1-b]thiazole-7-carboxylate (0.50 g, 1.41 mmol). Yield 0.34 g (74%), as a white solid. ¹H NMR (400 MHz, DMSO) δ 12.70 (br s, 1H), 8.72 (s, 1H), 8.67 (d, J=1.6 Hz, 1H), 8.29 (d, J=8.4 Hz, 1H), 8.11 (dd, J=1.6, 8.4 Hz, 1H), 7.98 (dd, J=2.4, 6.8 Hz, 1H), 7.26-7.10 (m, 2H), 2.36 (s, 3H). m/z: [ESI⁺] 327 (M+H)⁺.

### 2-(4-(Methylcarbamoyl)phenyl)benzo[d]imidazo[2,1-b]thiazole-7-carboxylic acid

Starting from ethyl 2-(4-(methylcarbamoyl)phenyl)benzo[d]imidazo[2,1-b]thiazole-7-carboxylate (10.00 g, 26.36 mmol). Yield 6.70 g (72%), as a white solid. ¹H NMR (400 MHz, DMSO) δ 13.15 (br s, 1H), 8.96 (s, 1H), 8.68 (d, J=1.6 Hz, 1H), 8.46 (q, J=4.6 Hz, 1H), 8.15 (dd, J=1.6, 8.4 Hz, 1H), 8.06 (d, J=8.4 Hz, 1H), 7.96 (d, J=8.6 Hz, 2H), 7.92 (d, J=8.6 Hz, 2H), 2.81 (d, J=4.6 Hz, 3H). m/z: [ESI⁺] 352 (M+H)⁺.

### Tert-butyl (3-aminopropyl)(2,2,2-trifluoroethyl)carbamate (Intermediate E)

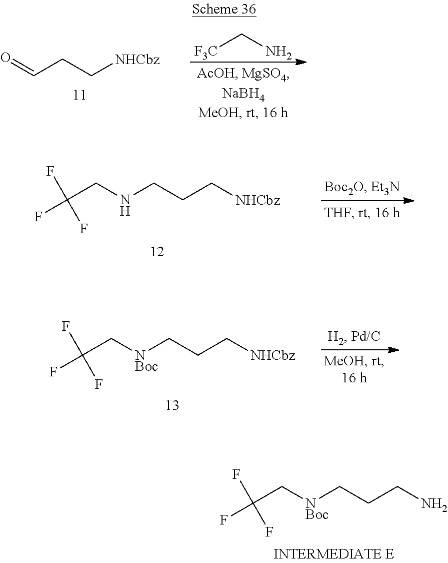 [SMILES: NCC(F)(F)F ; O=CCCNC(=O)OCc1ccccc1 ; O=C(NCCCNCC(F)(F)F)OCc1ccccc1 ; CC(C)(C)OC(=O)N(CCCNC(=O)OCc1ccccc1)CC(F)(F)F ; CC(C)(C)OC(=O)N(CCCN)CC(F)(F)F]

### Step 1: Benzyl (3-((2,2,2-trifluoroethyl)amino)propyl)carbamate

To a stirred solution of benzyl (3-oxopropyl)carbamate (1.50 g, 7.24 mmol) in methanol (15 mL) were added AcOH (0.53 g, 8.83 mmol), MgSO₄ (1.74 g, 14.46 mmol), 2,2,2-trifluoroethan-1-amine (1.08 g, 10.90 mmol) and sodium borohydride (0.55 g, 14.54 mmol). The resulting mixture was stirred for 16 h at room temperature under a nitrogen atmosphere. Upon completion, the resulting mixture was diluted with ethyl acetate (300 mL) and washed with saturated aqueous NaHCO₃ (3×30 mL). The organic layer was concentrated under reduced pressure. The residue was purified by reverse phase flash chromatography with the following conditions: Column: Spherical C18, 20-40 m, 330 g; Mobile Phase A: water (plus 10 mM NH₄HCO₃); Mobile Phase B: ACN; Flow rate: 80 mL/min; Gradient: 40% B-60% B in 20 min; Detector: UV 254/215 nm. The fractions containing desired product were collected and concentrated under reduced pressure to afford benzyl (3-((2,2,2-trifluoroethyl)amino)propyl)carbamate as a colorless oil.

Yield 1.20 g (57%). ¹H NMR (400 MHz, DMSO) δ 7.39-7.29 (m, 5H), 7.24 (d, J=5.8 Hz, 1H), 5.01 (s, 2H), 3.18 (q, J=10.2 Hz, 2H), 3.03 (q, J=6.6 Hz, 2H), 2.58 (t, J=7.0 Hz, 2H), 1.54-1.52 (M, 2H). m/z: [ESI⁺] 291 (M+H)⁺.

### Step 2: Tert-butyl (3-(((benzyloxy)carbonyl)amino)propyl)(2,2,2-trifluoroethyl)carbamate

To a stirred solution of benzyl (3-((2,2,2-trifluoroethyl)amino)propyl)carbamate (500 mg, 1.722 mmol) in THF (10 mL) were added triethylamine (349 mg, 3.449 mmol) and di-tert-butyl dicarbonate (564 mg, 2.584 mmol) at room temperature under a nitrogen atmosphere. After stirring for additional 16 h at room temperature under a nitrogen atmosphere, the resulting mixture was diluted with ethyl acetate (100 mL) and washed with water (3×20 mL). The organic layer was dried over anhydrous Na₂SO₄. After filtration, the filtrate was concentrated under reduced pressure. The residue was purified by reverse phase flash chromatography with the following conditions: Column: Spherical C18, 20-40 m, 330 g; Mobile Phase A: water (plus 10 mM NH₄HCO₃); Mobile Phase B: ACN; Flow rate: 80 mL/min; Gradient: 70% B-90% B in 20 min; Detector: UV 254/215 nm. The fractions containing desired product were collected and concentrated under reduced pressure to afford tert-butyl (3-(((benzyloxy)carbonyl)amino)propyl)(2,2,2-trifluoroethyl)carbamate as a colorless oil.

Yield 450 mg (67%). ¹H NMR (400 MHz, DMSO) δ 7.41-7.27 (m, 5H), 5.01 (s, 2H), 4.01 (q, J=9.4 Hz, 2H), 3.23 (t, J=7.6 Hz, 2H), 2.98 (q, J=6.4 Hz, 2H), 1.69-1.62 (m, 2H), 1.40 (s, 9H). m/z: [ESI⁺] 391 (M+H)⁺.

### Step 3: Tert-butyl (3-aminopropyl)(2,2,2-trifluoroethyl)carbamate

To a stirred solution of tert-butyl (3-(((benzyloxy)carbonyl)amino)propyl)(2,2,2-trifluoroethyl)carbamate (450 mg, 1.153 mmol) in methanol (10 mL) was added palladium on carbon (400 mg, 10% w/w) at room temperature under a nitrogen atmosphere. The resulting mixture was stirred for 16 h at room temperature under a hydrogen atmosphere (balloon). The resulting mixture was filtered and the filtrate was concentrated under reduced pressure to afford tert-butyl (3-aminopropyl)(2,2,2-trifluoroethyl)carbamate as a colorless oil.

Yield 270 mg (91%). ¹H NMR (400 MHz, DMSO) δ 4.01 (q, J=9.4 Hz, 2H), 3.27 (t, J=7.2 Hz, 2H), 2.53-2.48 (m, 2H), 1.62-1.54 (m, 2H), 1.41 (s, 9H). m/z: [ESI⁺] 257 (M+H)⁺.

### N¹-ethyl-N³-methyl-N¹-(2,2,2-trifluoroethyl)propane-1,3-diamine (Intermediate F) and N¹-ethyl-N¹-(2,2,2-trifluoroethyl)propane-1,3-diamine (Intermediate G)

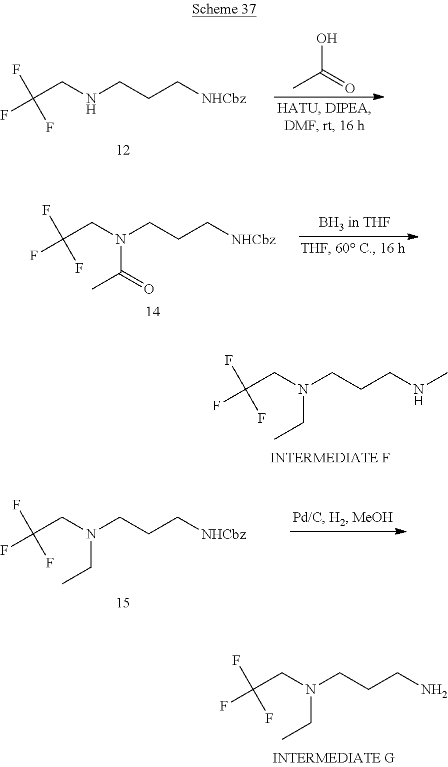

### Step 1: Benzyl (3-(N-(2,2,2-trifluoroethyl)acetamido)propyl)carbamate

To a stirred solution of AcOH (0.16 g, 2.66 mmol) in DMF (10 mL) were added HATU (1.18 g, 3.10 mmol), benzyl (3-((2,2,2-trifluoroethyl)amino)propyl)carbamate (0.60 g, 2.07 mmol) and DIPEA (0.80 g, 6.19 mmol) at room temperature under a nitrogen atmosphere. After stirring for additional 1 h at room temperature under a nitrogen atmosphere, the resulting mixture was purified by reverse phase flash chromatography with the following conditions: Column: Spherical C18, 20-40 μm, 330 g; Mobile Phase A: water (plus 10 mM NH₄HCO₃); Mobile Phase B: ACN; Flow rate: 80 mL/min; Gradient: 30% B-60% B in 20 min; Detector: UV 220/254 nm. The fractions containing desired product were collected and concentrated under reduced pressure to afford benzyl (3-(N-(2,2,2-trifluoroethyl)acetamido)propyl)carbamate as a colorless oil.

Yield: 0.50 g (72%). ¹H NMR (400 MHz, DMSO) δ 7.42-7.28 (m, 5H), 7.26 (t, J=5.8 Hz, 1H), 5.03 (s, 1.2H), 5.02 (s, 0.8H), 4.26 (q, J=9.6 Hz, 0.8H), 4.12 (q, J=9.6 Hz, 1.2H), 3.41-3.35 (m, 1.2H), 3.33-3.28 (m, 0.8H), 3.04 (q, J=6.4 Hz, 1.2H), 2.98 (q, J=6.4 Hz, 0.8H), 2.06 (s, 1.8H), 2.05 (s, 1.2H), 1.72-1.70 (m, 1.2H), 1.64-1.62 (m, 0.8H). m/z: [ESI⁺] 333 (M+H)⁺.

### Step 2: N¹-ethyl-N³-methyl-N¹-(2,2,2-trifluoroethyl)propane-1,3-diamine and Benzyl (3-(ethyl(2,2,2-trifluoroethyl)amino)propyl)carbamate

To a stirred solution of benzyl (3-(N-(2,2,2-trifluoroethyl)acetamido)propyl)carbamate (0.50 g, 1.50 mmol) in THF (10 mL) was added a 1M solution of borane in THF (10 mL, 10.00 mmol) under a nitrogen atmosphere. The resulting solution was stirred for overnight at 60° C. under a nitrogen atmosphere. The mixture was cooled down to room temperature followed by the addition of methanol (10 mL). The resulting mixture was stirred for additional 1 h at 60° C. After cooling down to room temperature, the resulting mixture was concentrated under reduced pressure to afford a mixture of N¹-ethyl-N³-methyl-N¹-(2,2,2-trifluoroethyl)propane-1,3-diamine and benzyl (3-(ethyl(2,2,2-trifluoroethyl)amino)propyl)carbamate as a colorless oil with a ratio of 1:4.

Crude yield: 0.32 g. Benzyl (3-(ethyl(2,2,2-trifluoroethyl)amino)propyl)carbamate. m/z: [ESI⁺] 319 (M+H)⁺. N¹-ethyl-N³-methyl-N¹-(2,2,2-trifluoroethyl)propane-1,3-diamine. m/z: [ESI⁺] 199 (M+H)⁺.

### Step 3: N¹-ethyl-N³-methyl-N¹-(2,2,2-trifluoroethyl)propane-1,3-diamine and N¹-ethyl-N¹-(2,2,2-trifluoroethyl)propane-1,3-diamine

To a stirred solution of the above mixture (0.32 g) in methanol (10 mL) was added palladium on carbon (300 mg, 10% w/w) at room temperature under a nitrogen atmosphere. The resulting mixture was stirred for 16 h at room temperature under a hydrogen atmosphere (balloon). The resulting mixture was filtered and the filtrate was concentrated under reduced pressure to afford a mixture of N¹-ethyl-N³-methyl-N′-(2,2,2-trifluoroethyl)propane-1,3-diamine and N¹-ethyl-N¹-(2,2,2-trifluoroethyl)propane-1,3-diamine as a colorless oil (ratio 4:1).

Crude yield 0.27 g. m/z: [ESI⁺] 185 (M+H)⁺.

### (2-(p-Tolyl)-1H-benzo[d]imidazol-5-yl)methanamine (Intermediate H)

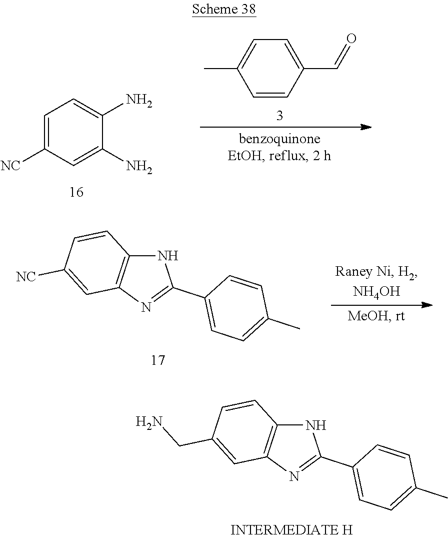

### Step 1: 2-(p-Tolyl)-1H-benzo[d]imidazole-5-carbonitrile

To a stirred solution of 4-methylbenzaldehyde (1.80 g, 14.98 mmol) in ethanol (40 mL) were added 3,4-diaminobenzonitrile (1.99 g, 14.95 mmol) and benzoquinone (1.62 g, 14.99 mmol) at room temperature under a nitrogen atmosphere. The resulting mixture was refluxed for 2 h under a nitrogen atmosphere. The resulting mixture was cooled down to room temperature and concentrated under reduced pressure. The residue was purified by silica gel column chromatography, eluted with 1%-70% ethyl acetate in petroleum ether to afford 2-(p-tolyl)-1H-benzo[d]imidazole-5-carbonitrile as a brown solid.

Yield 3.00 g (86%). ¹H NMR (400 MHz, DMSO) δ 13.39 (br s, 0.4H), 13.37 (br s, 0.6H), 8.62 (s, 1H), 8.10 (d, J=8.0 Hz, 2H), 7.81 (d, J=8.4 Hz, 0.4H), 7.68 (d, J=8.4 Hz, 0.6H), 7.60 (d, J=8.4 Hz, 0.6H), 7.58 (d, J=8.4 Hz, 0.4H), 7.39 (d, J=8.0 Hz, 2H), 2.40 (s, 3H). (tautomers). m/z: [ESI⁺] 234 (M+H)⁺.

### Step 2: (2-(p-Tolyl)-1H-benzo[d]imidazol-5-yl)methanamine

To a stirred solution of 2-(p-tolyl)-1H-benzo[d]imidazole-5-carbonitrile (2.00 g, 8.57 mmol) in methanol (80 mL) were added a 25% solution of NH₄OH in water (9 mL, 57.68 mmol) and Raney Ni (1.00 g, 17.04 mmol) at room temperature under a nitrogen atmosphere. The resulting mixture was stirred for 3 h at room temperature under a hydrogen atmosphere (balloon). The resulting mixture was filtered. The filtered cake was washed with methanol (3×30 mL). The combined filtrates were concentrated under reduced pressure to afford (2-(p-tolyl)-1H-benzo[d]imidazol-5-yl)methanamine as a brown solid.

Yield 2.00 g, (98%). ¹H NMR (400 MHz, DMSO) δ 8.06 (s, J=8.2 Hz, 2H), 7.52 (s, 1H), 7.50 (d, J=8.4 Hz, 1H), 7.35 (d, J=8.2 Hz, 2H), 7.16 (d, J=8.4 Hz, 1H), 3.83 (s, 2H), 2.38 (s, 3H). m/z: [ESI⁺] 238 (M+H)⁺.

### 2-(m-Tolyl)benzo[d]imidazo[2,1-b]thiazol-7-aminium (Intermediate I)

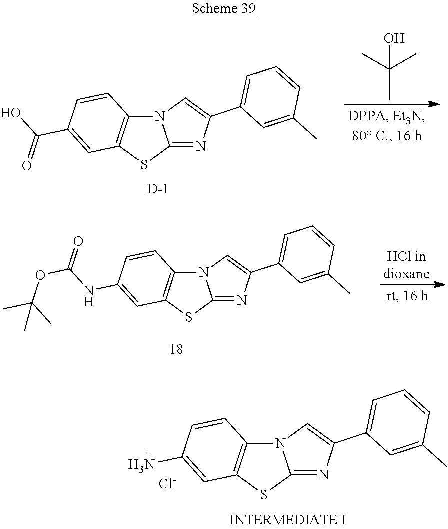

### Step 1: Tert-butyl (2-(m-tolyl)benzo[d]imidazo[2,1-b]thiazol-7-yl)carbamate

To a stirred solution of 2-(m-tolyl)benzo[d]imidazo[2,1-b]thiazole-7-carboxylic acid (4.00 g, 12.97 mmol) in tert-butanol (80 mL) were added triethylamine (2.63 g, 25.94 mmol) and diphenyl phosphorazidate (5.35 g, 19.44 mmol) at room temperature under a nitrogen atmosphere. The resulting mixture was stirred for 16 h at 80° C. under a nitrogen atmosphere. The resulting mixture was cooled down to room temperature and concentrated under reduced pressure. The residue was purified by silica gel column chromatography, eluted with 1%-25% ethyl acetate in petroleum ether to afford tert-butyl (2-(m-tolyl)benzo[d]imidazo[2,1-b]thiazol-7-yl)carbamate as an off-white solid.

Yield 0.60 g (12%). ¹H NMR (400 MHz, CDCl₃) δ 7.98-7.95 (m, 1H), 7.91 (s, 1H), 7.74 (d, J=2.0 Hz, 1H), 7.65 (d, J=7.8 Hz, 1H), 7.49 (d, J=8.6 Hz, 1H), 7.35-7.29 (m, 2H), 7.13 (d, J=7.6 Hz, 1H), 6.74 (s, 1H), 2.43 (s, 3H), 1.56 (s, 9H). m/z: [ESI⁺] 380 (M+H)⁺.

### Step 2: 2-(m-Tolyl)benzo[d]imidazo[2,1-b]thiazol-7-aminium chloride

A solution of tert-butyl (2-(m-tolyl)benzo[d]imidazo[2,1-b]thiazol-7-yl)carbamate (100 mg, 0.264 mmol) in a 4M solution of HCl (gas) in 1,4-dioxane (10 mL) was stirred for 16 h at room temperature under a nitrogen atmosphere. The resulting mixture was concentrated under reduced pressure to afford 2-(m-tolyl)benzo[d]imidazo[2,1-b]thiazol-7-aminium chloride as an off-white solid.

Crude yield 100 mg. ¹H NMR (400 MHz, DMSO) δ 8.96 (s, 1H), 8.15-8.09 (m, 2H), 7.71 (s, 1H), 7.67 (d, J=7.8 Hz, 1H), 7.60 (dd, J=8.5, 2.0 Hz, 1H), 7.36 (dd, J=1.6, 7.6 Hz, 1H), 7.17 (d, J=7.6 Hz, 1H), 2.37 (s, 3H). m/z: [ESI⁺] 280 (M+H)⁺.

### 5-Bromo-1H-indol-2-aminium chloride (Intermediate J)

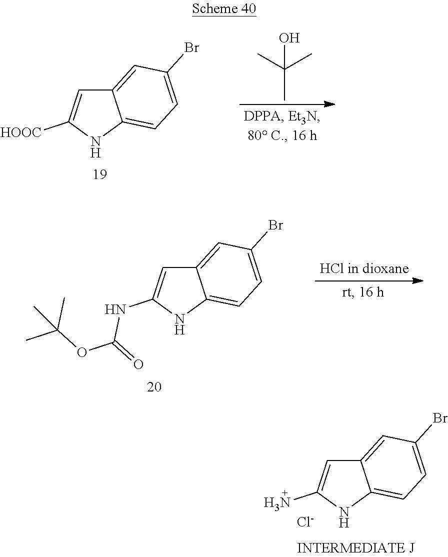

### Step 1: Tert-butyl (5-bromo-1H-indol-2-yl)carbamate

To a stirred solution of 5-bromo-1H-indole-2-carboxylic acid (3.00 g, 12.50 mmol) in tert-butanol (12 mL) were added triethylamine (2.53 g, 24.99 mmol) and diphenyl azidophosphate (5.16 g, 18.75 mmol) at room temperature under a nitrogen atmosphere. The resulting mixture was stirred for 16 h at 80° C. under a nitrogen atmosphere. The resulting mixture was cooled down to room temperature and concentrated under reduced pressure. The residue was purified by silica gel column chromatography, eluted with 1%-25% ethyl acetate in petroleum ether to afford tert-butyl (5-bromo-1H-indol-2-yl)carbamate as an off-white solid.

Yield 2.40 g (62%). ¹H NMR (400 MHz, DMSO) δ 10.90 (br s, 1H), 10.15 (br s, 1H), 7.48 (d, J=2.0 Hz, 1H), 7.35 (d, J=8.6 Hz, 1H), 7.01 (dd, J=2.0, 8.6 Hz, 1H), 5.88 (d, J=2.0 Hz, 1H), 1.51 (s, 9H). m/z: [ESI⁺] 311, 313 (M+H)⁺.

### Step 2: 5-Bromo-1H-indol-2-aminium chloride

A solution of tert-butyl (5-bromo-1H-indol-2-yl)carbamate (2.00 g, 6.43 mmol) in a 4M solution of HCl (gas) in dioxane (20 mL) was stirred for 16 h at room temperature under a nitrogen atmosphere. The resulting mixture was concentrated under reduced pressure to afford 5-bromo-1H-indol-2-aminium chloride as a brown solid, which was used in the next step without further purification.

Crude yield 1.00 g (crude). m/z: [ESI⁺] 212 (M+H)⁺.

### (R)-(1-(3-aminopropyl)pyrrolidin-2-yl)methanol dichloride (Intermediate K)

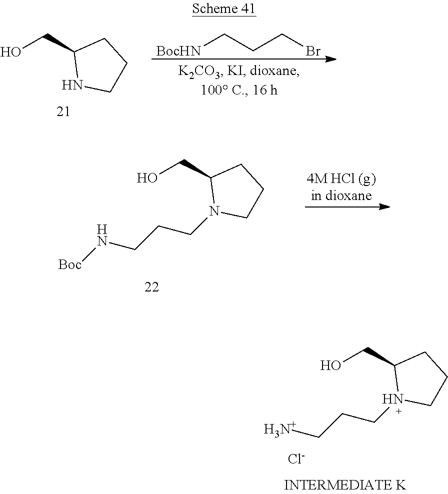 [SMILES: OC[C@H]1CCCN1 ; CC(C)(C)OC(=O)NCCCBr ; CC(C)(C)OC(=O)NCCCN1CCC[C@@H]1CO ; [NH3+]CCCN1CCC[C@@H]1CO ; [Cl-]]

### Step 1: Tert-butyl (R)-(3-(2-(hydroxymethyl)pyrrolidin-1-yl)propyl)carbamate

To a stirred solution of (R)-pyrrolidin-2-ylmethanol (0.50 g, 4.94 mmol) in dioxane (10 mL) were added K₂CO₃ (1.37 g, 9.91 mmol), KI (0.41 g, 2.47 mmol) and tert-butyl (3-bromopropyl)carbamate (4.71 g, 19.78 mmol) at room temperature under a nitrogen atmosphere. The resulting mixture was stirred for 16 h at 100° C. under a nitrogen atmosphere. Upon completion, the resulting mixture was concentrated under reduced pressure and the residue was purified by Prep-HPLC (mass directed) with the following conditions: Column: Sunfire prep C18 column, 30×150 mm, 5 μm; Mobile Phase A: water (plus 10 mmol/L NH₄HCO₃); Mobile Phase B: ACN; Flow rate: 60 mL/min; Gradient: 20% B-40% B in 8 min. Desired fractions were collected and concentrated under reduced pressure to afford tert-butyl (R)-(3-(2-(hydroxymethyl)pyrrolidin-1-yl)propyl)carbamate as a brown oil.

Yield 0.24 g (19%). ¹H NMR (400 MHz, CDCl₃) δ 4.95-4.91 (m, 1H), 3.70-3.62 (m, 1H), 3.50-3.40 (m, 1H), 3.27-3.23 (m, 3H), 2.86-2.83 (m, 1H), 2.63-2.60 (m, 1H), 2.37-2.34 (m, 1H), 2.32-2.20 (m, 1H), 1.78-1.46 (m, 4H), 1.46 (s, 9H). m/z: [ESI⁺] 259 (M+H)⁺.

### Step 2: (R)-(1-(3-aminopropyl)pyrrolidin-2-yl)methanol dichloride

A solution of tert-butyl (R)-(3-(2-(hydroxymethyl)pyrrolidin-1-yl)propyl)carbamate (240 mg, 0.929 mmol) in a 4M solution of HCl (gas) in dioxane (10 mL) was stirred for 16 h at room temperature under a nitrogen atmosphere. The resulting mixture was concentrated under reduced pressure to afford (R)-(1-(3-aminopropyl)pyrrolidin-2-yl)methanol dichloride as a yellow oil, which was used in the next step without further purification.

Crude yield 200 mg. ¹H NMR (400 MHz, CD₃OD) δ 3.95-3.92 (m, 1H), 3.83-3.66 (m, 3H), 3.64-3.54 (m, 1H), 3.28-3.17 (m, 2H), 3.17-3.02 (m, 2H), 2.33-2.08 (m, 2H), 2.03-1.87 (m, 2H). m/z: [ESI⁺] 259 (M+H)⁺.

### 2-bromo-N-(3-(piperidin-1-yl)propyl)benzo[d]imidazo[2,1-b]thiazole-7-carboxamide

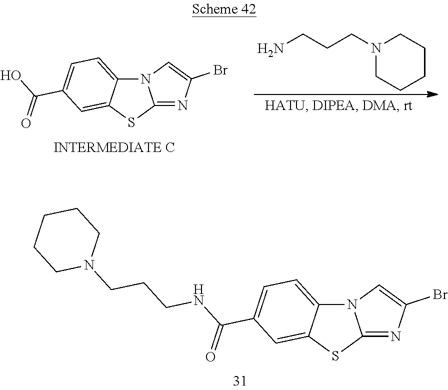 [SMILES: O=C(O)c1ccc2c(c1)sc1nc(Br)cn12 ; NCCCN1CCCCC1 ; O=C(NCCCN1CCCCC1)c1ccc2c(c1)sc1nc(Br)cn12]

To a stirred solution of 2-bromobenzo[d]imidazo[2,1-b]thiazole-7-carboxylic acid (1.00 g, 3.37 mmol) and O-(7-azabenzotriazol-1-yl)-N,N,N′,N′-tetramethyluronium hexafluorophosphate (HATU) (1.92 g, 5.05 mmol) in N,N-dimethylacetamide (15 mL) were added 3-(piperidin-1-yl)propan-1-amine (0.62 g, 4.36 mmol) and N-ethyl-N-isopropylpropan-2-amine (1.30 g, 10.06 mmol) at room temperature. The resulting mixture was stirred for 16 h at room temperature under a nitrogen atmosphere. The reaction was diluted with water (50 mL). The precipitated solids were collected by filtration and the filter cake was washed with water (3×10 mL) and oven dried to afford 2-bromo-N-(3-(piperidin-1-yl)propyl)benzo[d]imidazo[2,1-b]thiazole-7-carboxamide as an off-white solid.

Yield 1.30 g (91%). ¹H NMR (400 MHz, DMSO) δ 8.64 (t, J=5.6 Hz, 1H), 8.56 (s, 1H), 8.51 (d, J=1.6 Hz, 1H), 8.09 (d, J=8.4 Hz, 1H), 8.01 (dd, J=1.6, 8.4 Hz, 1H), 3.33-3.28 (m, 2H), 2.42-2.24 (m, 6H), 1.74-1.66 (m, 2H), 1.53-1.46 (m, 4H), 1.42-1.35 (m, 2H). m/z: [ESI⁺] 421, 423 (M+H)⁺.

### tert-butyl (3-cyclopropyl-4-(7-((3-(piperidin-1-yl)propyl)carbamoyl)benzo[d]imidazo[2,1-b]thiazol-2-yl)benzyl)carbamate

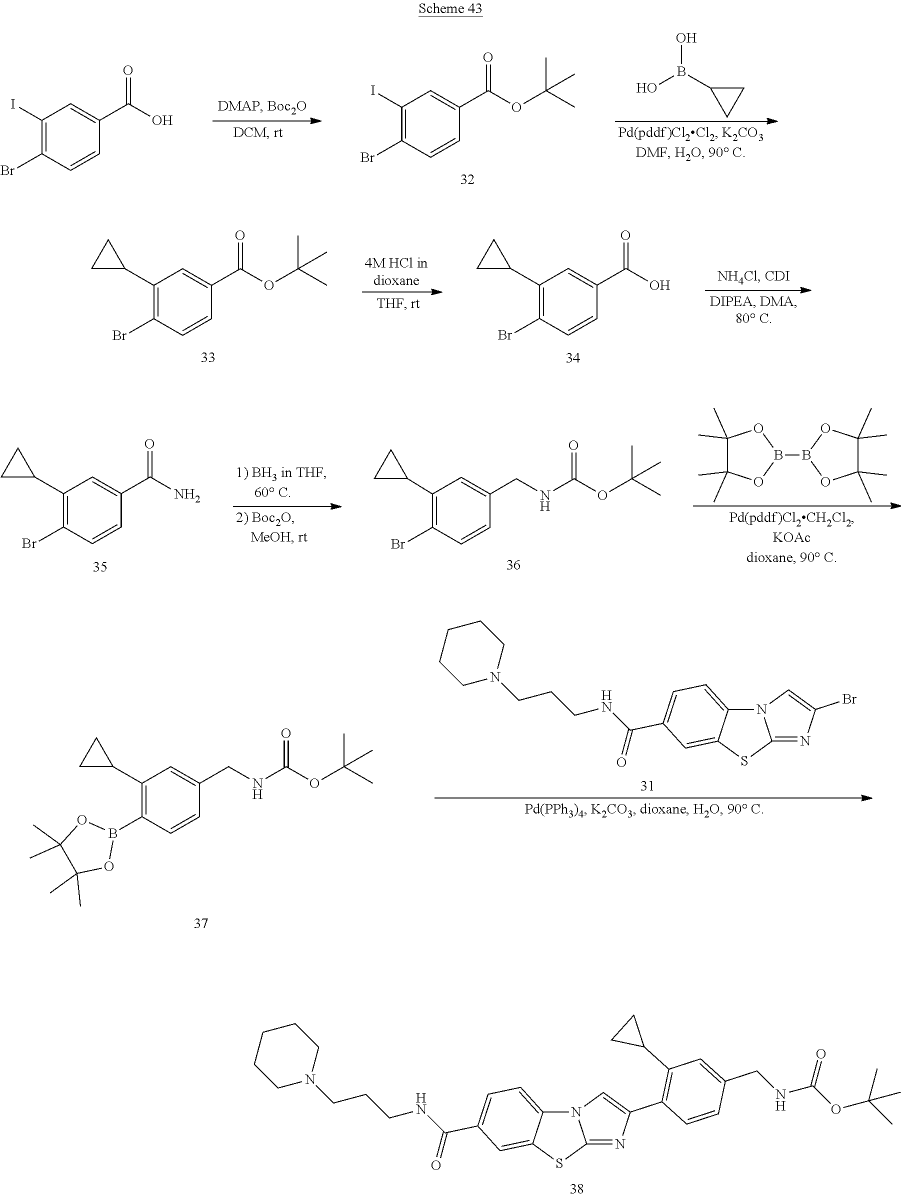

### tert-butyl 4-bromo-3-iodobenzoate

To a stirred solution of 4-bromo-3-iodobenzoic acid (3.26 g, 9.97 mmol) and N,N-dimethylpyridin-4-amine (0.29 g, 2.37 mmol) in dichloromethane (70 mL) was added di-tert-butyl dicarbonate (4.35 g, 19.94 mmol) at room temperature. The resulting solution was stirred for 16 h at room temperature under a nitrogen atmosphere. The resulting solution was concentrated under reduced pressure. The residue was purified by silica gel column chromatography, eluting with 1%-10% ethyl acetate in petroleum ether to afford tert-butyl 4-bromo-3-iodobenzoate as a light yellow oil.

Yield 3.50 g (92%). ¹H NMR (400 MHz, CDCl₃) δ 8.44 (d, J=2.0 Hz, 1H), 7.81 (dd, J=2.0, 8.4 Hz, 1H), 7.68 (d, J=8.4 Hz, 1H), 1.61 (s, 9H). No MS signal.

### tert-butyl 4-bromo-3-cyclopropylbenzoate

To a stirred solution of tert-butyl 4-bromo-3-iodobenzoate (1.56 g, 4.07 mmol) and cyclopropylboronic acid (0.45 g, 5.30 mmol) in N,N-dimethylformamide (20 mL) and water (4 mL) were added potassium carbonate (1.13 g, 8.19 mmol) and 1,1′-bis(diphenylphosphino)ferrocene-palladium (II) dichloride dichloromethane complex (0.50 g, 0.61 mmol) portion-wise at room temperature. The resulting mixture was stirred for 16 h at 90° C. under a nitrogen atmosphere. Upon completion, the resulting mixture was cooled to room temperature and filtered. The filter cake was washed with methanol (3×10 mL). The filtrate was concentrated under reduced pressure. The residue was purified by reverse phase flash chromatography with the following conditions: Column: Spherical C18, 20-40 m, 330 g; Mobile Phase A: water (plus 10 mM ammonium bicarbonate); Mobile Phase B: acetonitrile; Flow rate: 80 m/min; Gradient: 75%-95% B in 20 min; Detector: UV 254/220 nm. The fractions containing desired product were collected and concentrated under reduced pressure to afford tert-butyl 4-bromo-3-cyclopropylbenzoate as a yellow oil.

Yield 0.41 g (34%). ¹H NMR (400 MHz, CDCl₃) δ 7.63 (d, J=8.0 Hz, 1H), 7.61 (s, 1H), 7.58 (d, J=8.0 Hz, 1H), 2.23-2.14 (m, 1H), 1.60 (s, 9H), 1.09-1.02 (m, 2H), 0.79-0.70 (m, 2H). No MS signal.

### 4-bromo-3-cyclopropylbenzoic acid

To a stirred solution of tert-butyl 4-bromo-3-cyclopropylbenzoate (3.40 g, 11.44 mmol) in tetrahydrofuran (50 mL) was added a of 4.0 M solution of hydrogen chloride in dioxane (10 mL) drop-wise at room temperature. The resulting mixture was stirred for 16 h at room temperature under a nitrogen atmosphere. The resulting mixture was concentrated under reduced pressure. The residue was purified by reverse phase flash chromatography with the following conditions: Column: Spherical C18, 20-40 m, 330 g; Mobile Phase A: water (plus 10 mM formic acid); Mobile Phase B: acetonitrile; Flow rate: 80 m/min; Gradient: 40%-60% B in 20 min; Detector: UV 254/220 nm. The fractions containing desired product were collected and concentrated under reduced pressure to afford 4-bromo-3-cyclopropylbenzoic acid as a light brown solid.

Yield 1.53 g (55%). ¹H NMR (400 MHz, DMSO) δ 13.01 (br s, 1H), 7.73 (d, J=8.0 Hz, 1H), 7.65 (dd, J=2.0, 8.0, Hz, 1H), 7.51 (d, J=2.0 Hz, 1H), 2.25-2.09 (m, 1H), 1.10-0.96 (m, 2H), 0.76-0.61 (m, 2H). m/z: [ESI] 239, 241 (M−H)⁻.

### 4-bromo-3-cyclopropylbenzamide

To a stirred solution of 4-bromo-3-cyclopropylbenzoic acid (1.53 g, 6.35 mmol) and 1,1′-carbonyldiimidazole (1.54 g, 9.52 mmol) in N,N-dimethylacetamide (20 mL) were added N-ethyl-N-isopropylpropan-2-amine (2.46 g, 19.04 mmol) and ammonium chloride (1.02 g, 19.04 mmol) at room temperature. The resulting mixture was stirred for 16 h at 80° C. under a nitrogen atmosphere. Upon completion, the resulting mixture was cooled to room temperature and concentrated under reduced pressure. The residue was purified by reverse phase flash chromatography with the following conditions: Column: Spherical C18, 20-40 m, 330 g; Mobile Phase A: water (plus 10 mM ammonium bicarbonate); Mobile Phase B: acetonitrile; Flow rate: 80 m/min; Gradient: 45%-65% B in 20 min; Detector: UV 254/220 nm. The fractions containing desired product were collected and concentrated under reduced pressure to afford 4-bromo-3-cyclopropylbenzamide as a light yellow solid.

Yield 0.71 g (47%). ¹H NMR (400 MHz, CDCl₃) δ 7.63 (d, J=8.0 Hz, 1H), 7.48-7.40 (m, 2H), 6.01 (br s, 2H), 2.24-2.15 (m, 1H), 1.12-1.04 (m, 2H), 0.82-0.71 (m, 2H). m/z: [ESI⁺] 240, 242 (M+H)⁺.

### tert-butyl (4-bromo-3-cyclopropylbenzyl)carbamate

To a stirred solution of 4-bromo-3-cyclopropylbenzamide (0.71 g, 2.96 mmol) in tetrahydrofuran (5 mL) was added borane-tetrahydrofuran complex (1 M in THF, 5.63 mL, 5.63 mmol) at room temperature. The resulting solution was stirred for 2 h at 60° C. under a nitrogen atmosphere. Upon completion, the resulting solution was cooled to room temperature and quenched by the addition of methanol (5 mL). To the above mixture was added di-tert-butyl dicarbonate (1.94 g, 8.89 mmol) at room temperature. The resulting solution was stirred for additional 16 h at room temperature. The resulting solution was concentrated under reduced pressure. The residue was purified by reverse phase flash chromatography with the following conditions: Column: Spherical C18, 20-40 m, 330 g; Mobile Phase A: water (plus 10 mM ammonium bicarbonate); Mobile Phase B: acetonitrile; Flow rate: 80 m/min; Gradient: 80%-95% B in 20 min; Detector: 254/220 nm. The fractions containing desired product were collected and concentrated under reduced pressure to afford tert-butyl (4-bromo-3-cyclopropylbenzyl)carbamate as a light yellow solid.

Yield 0.50 g (52%). ¹H NMR (400 MHz, DMSO) δ 7.51 (d, J=8.0 Hz, 1H), 7.36 (t, J=6.4 Hz, 1H), 7.00-6.83 (m, 2H), 4.03 (t, J=6.4 Hz, 2H), 2.22-2.10 (m, 1H), 1.39 (s, 9H), 0.99-0.89 (m, 2H), 0.63-0.57 (m, 2H). m/z: [ESI] 324, 326 (M−H)⁻.

### tert-butyl (3-cyclopropyl-4-(4,4,5,5-tetramethyl-1,3,2-dioxaborolan-2-yl)benzyl)carbamate

To a stirred solution of tert-butyl (4-bromo-3-cyclopropylbenzyl)carbamate (200 mg, 0.613 mmol) and bis(pinacolato)diboron (467 mg, 1.839 mmol) in 1,4-dioxane (5 mL) were added potassium acetate (180 mg, 1.839 mmol) and 1,1′-bis(diphenylphosphino)ferrocene-palladium (II) dichloride dichloromethane complex (49 mg, 0.061 mmol) portion-wise at room temperature. The resulting mixture was stirred for 3 h at 90° C. under a nitrogen atmosphere. After cooling to room temperature, the resulting mixture was filtered and the filter cake was washed with methanol (3×10 mL). The filtrate was concentrated under reduced pressure. The residue was purified by reverse phase flash chromatography with the following conditions: Column: Spherical C18, 20-40 m, 330 g; Mobile Phase A: water (plus 10 mM ammonium bicarbonate); Mobile Phase B: acetonitrile; Flow rate: 80 m/min; Gradient: 75%-95% B in 20 min; Detector: UV 254/220 nm. The fractions containing desired product were collected and concentrated under reduced pressure to afford tert-butyl (3-cyclopropyl-4-(4,4,5,5-tetramethyl-1,3,2-dioxaborolan-2-yl)benzyl)carbamate as a brown solid

Yield 120 mg (52%). ¹H NMR (400 MHz, DMSO) δ 7.53 (d, J=7.6 Hz, 1H), 7.34 (t, J=6.4 Hz, 1H), 6.98 (dd, J=1.6, 7.6 Hz, 1H), 6.69 (d, J=1.6 Hz, 1H), 4.06 (d, J=6.4 Hz, 2H), 2.72-2.56 (m, 1H), 1.39 (s, 9H), 1.30 (s, 12H), 0.99-0.90 (m, 2H), 0.65-0.55 (m, 2H). M/z: [ESI⁺] 374 (M+H)⁺.

### tert-butyl (3-cyclopropyl-4-(7-((3-(piperidin-1-yl)propyl)carbamoyl)benzo[d]imidazo[2,1-b]thiazol-2-yl)benzyl)carbamate

To a stirred solution of 2-bromo-N-(3-(piperidin-1-yl)propyl)benzo[d]imidazo[2,1-b]thiazole-7-carboxamide (100 mg, 0.237 mmol), potassium carbonate (100 mg, 0.724 mmol) and tert-butyl (3-cyclopropyl-4-(4,4,5,5-tetramethyl-1,3,2-dioxaborolan-2-yl)benzyl)carbamate (120 mg, 0.321 mmol) in 1,4-dioxane (4 mL) and water (4 mL) was added tetrakis(triphenylphosphine)palladium (0) (30 mg, 0.026 mmol) portion-wise at room temperature. The resulting mixture was stirred for 3 h at 90° C. under a nitrogen atmosphere. Upon completion, the mixture was cooled to room temperature and purified by reverse phase flash chromatography with the following conditions: Column: Spherical C18, 20-40 m, 120 g; Mobile Phase A: water (plus 10 mM formic acid); Mobile Phase B: acetonitrile; Flow rate: 50 m/min; Gradient: 30%-50% B in 20 min; Detector: UV 254/220 nm. The fractions containing desired product were collected and concentrated under reduced pressure to afford tert-butyl (3-cyclopropyl-4-(7-((3-(piperidin-1-yl)propyl)carbamoyl)benzo[d]imidazo[2,1-b]thiazol-2-yl)benzyl)carbamate as a yellow solid.

Yield 100 mg (72%). ¹H NMR (400 MHz, DMSO) δ 8.65 (t J=6.0 Hz, 1H), 8.63 (s, 1H), 8.49 (d, J=1.6 Hz, 1H), 8.22 (d, J=8.4 Hz, 1H), 8.02 (dd, J=1.6, 8.4 Hz, 1H), 7.85 (d, J=8.0 Hz, 1H), 7.37 (t, J=6.0 Hz, 1H), 7.13 (dd, J=2.0, 8.0 Hz, 1H), 7.00 (d, J=2.0 Hz, 1H), 4.13 (d, J=6.0 Hz, 2H), 3.49-3.40 (m, 2H), 2.60-2.55 (m, 6H), 1.82-1.70 (m, 3H), 1.54-1.48 (m, 4H), 1.42 (s, 9H), 1.47-1.36 (m, 2H), 1.12-1.03 (m, 2H), 0.79-0.63 (m, 2H). M/z: [ESI⁺] 588 (M+H)⁺.

### tert-butyl (2-chloro-6-fluoro-4-(7-((3-(piperidin-1-yl)propyl)carbamoyl)benzo[d]imidazo[2,1-b]thiazol-2-yl)benzyl)carbamate

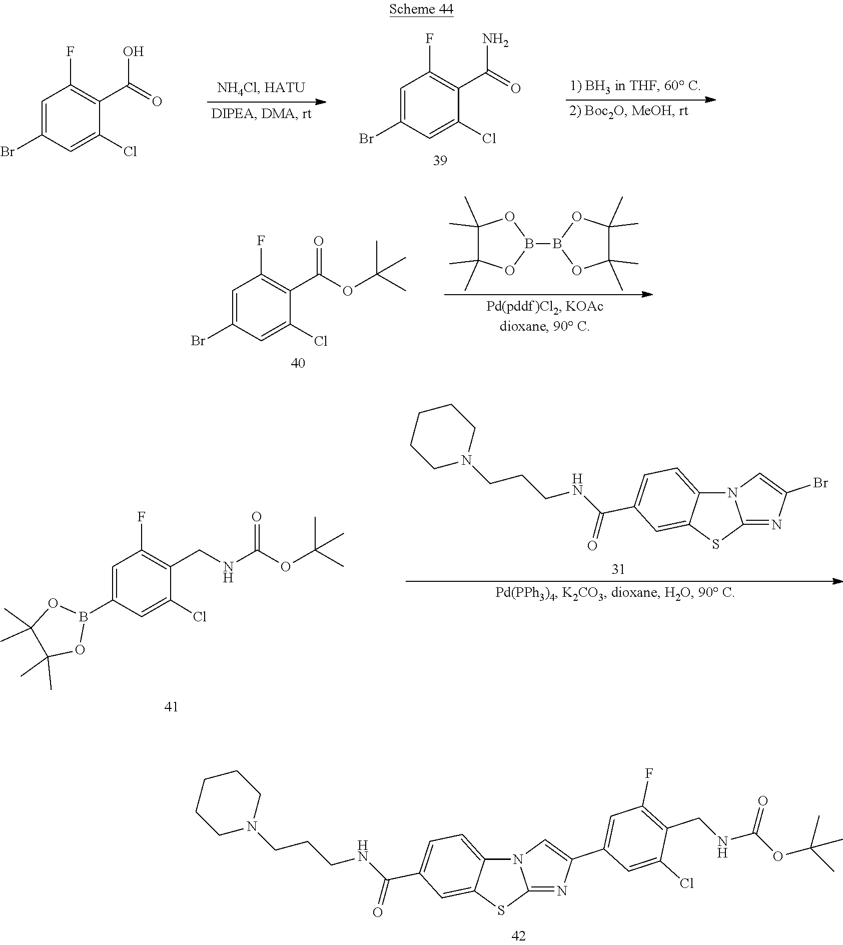

### 4-bromo-2-chloro-6-fluorobenzamide

Compound 4-bromo-2-chloro-6-fluorobenzamide was prepared from 4-bromo-2-chloro-6-fluorobenzoic acid (3.00 g, 11.84 mmol) and ammonium chloride (6.30 g, 117.78 mmol) following a similar procedure to that described for the synthesis of 2-bromo-N-(3-(piperidin-1-yl)propyl)benzo[d]imidazo[2,1-b]thiazole-7-carboxamide, and was isolated as an off-white solid.

Yield 2.60 g (87%). ¹H NMR (400 MHz, CDCl₃) δ 7.44 (d, J=1.6 Hz, 1H), 7.27 (dd, J=1.6, 8.4 Hz, 1H), 6.46 (br s, 1H), 5.96 (br s, 1H). m/z: [ESI⁺] 252, 254, 256 (M+H)⁺.

### tert-butyl (4-bromo-2-chloro-6-fluorobenzyl)carbamate

Compound tert-butyl (4-bromo-2-chloro-6-fluorobenzyl)carbamate was prepared from 4-bromo-2-chloro-6-fluorobenzamide (2.60 g, 10.30 mmol) following a similar procedure to that described for the synthesis of tert-butyl (4-bromo-3-cyclopropylbenzyl)carbamate and was isolated as an off-white solid.

Yield 709 mg (21%). ¹H NMR (400 MHz, CDCl₃) δ 7.40 (d, J=2.0 Hz, 1H), 7.22 (dd, J=2.0, 8.8 Hz, 1H), 4.91 (t, J=5.4 Hz, 1H), 4.46 (d, J=5.4 Hz, 2H), 1.46 (s, 9H). m/z: [ESI⁺] 338, 340, 342 (M+H)⁺.

### tert-butyl (2-chloro-6-fluoro-4-(4,4,5,5-tetramethyl-1,3,2-dioxaborolan-2-yl)benzyl)carbamate

Compound tert-butyl (2-chloro-6-fluoro-4-(4,4,5,5-tetramethyl-1,3,2-dioxaborolan-2-yl)benzyl)carbamate was prepared from tert-butyl (4-bromo-2-chloro-6-fluorobenzyl)carbamate (700 mg, 2.07 mmol) following a similar procedure to that described for the synthesis of tert-butyl (3-cyclopropyl-4-(4,4,5,5-tetramethyl-1,3,2-dioxaborolan-2-yl)benzyl)carbamate and was isolated as a yellow solid.

Yield 297 mg (37%). m/z: [ESI⁺] 386, 388 (M+H)⁺.

### tert-butyl (2-chloro-6-fluoro-4-(7-((3-(piperidin-1-yl)propyl)carbamoyl)benzo[d]imidazo[2,1-b]thiazol-2-yl)benzyl)carbamate

Compound tert-butyl (2-chloro-6-fluoro-4-(7-((3-(piperidin-1-yl)propyl)carbamoyl)benzo[d]imidazo[2,1-b]thiazol-2-yl)benzyl)carbamate was prepared from 2-bromo-N-(3-(piperidin-1-yl)propyl)benzo[d]imidazo[2,1-b]thiazole-7-carboxamide (200 mg, 0.475 mmol) and tert-butyl (2-chloro-6-fluoro-4-(4,4,5,5-tetramethyl-1,3,2-dioxaborolan-2-yl)benzyl)carbamate (265 mg, 0.688 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate and was isolated as a yellow solid.

Yield 200 mg (70%). ¹H NMR (400 MHz, DMSO) δ 8.99 (s, 1H), 8.69 (t, J=5.6 Hz, 1H), 8.51 (s, 1H), 8.04 (d, J=8.8 Hz, 1H), 7.98 (d, J=8.4 Hz, 1H), 7.78 (s, 1H), 7.62 (d, J=10.4 Hz, 1H), 7.24-7.21 (m, 1H), 4.28 (d, J=5.2 Hz, 2H), 3.39-3.29 (m, 2H), 2.49-2.44 (m, 6H), 1.80-1.71 (m, 2H), 1.60-1.50 (m, 4H), 1.40 (s, 9H), 1.47-1.36 (m, 2H). m/z: [ESI⁺] 600, 602 (M+H)⁺.

### tert-butyl (2,6-difluoro-4-(7-((3-(piperidin-1-yl)propyl)carbamoyl)benzo[d]imidazo[2,1-b]thiazol-2-yl)benzyl)carbamate

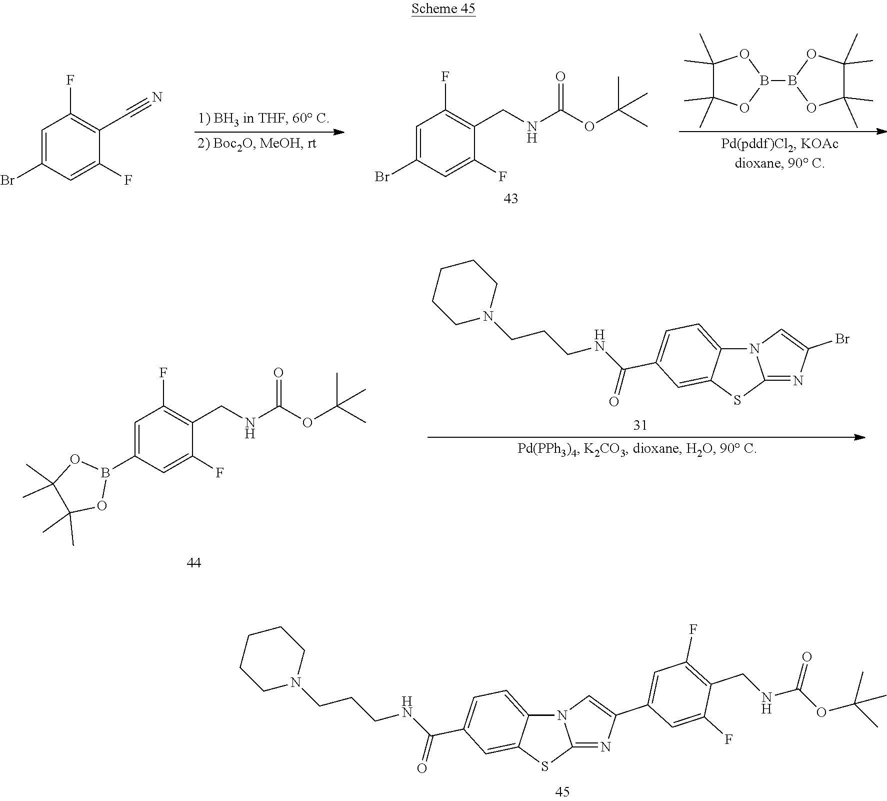

### tert-butyl (4-bromo-2,6-difluorobenzyl)carbamate

Compound tert-butyl (4-bromo-2,6-difluorobenzyl)carbamate was prepared from 4-bromo-2,6-difluorobenzonitrile (4.00 g, 18.35 mmol) following a similar procedure to that described for the synthesis of tert-butyl (4-bromo-3-cyclopropylbenzyl)carbamate, and was isolated as an off-white solid.

Yield 4.20 g (71%). ¹H NMR (400 MHz, CDCl₃) δ 7.11 (d, J=6.8 Hz, 2H), 4.88 (br s, 1H), 4.38 (s, 2H), 1.45 (s, 9H). m/z: [ESi⁺] 266, 268 (M+H−56)⁺.

### tert-butyl (2,6-difluoro-4-(4,4,5,5-tetramethyl-1,3,2-dioxaborolan-2-yl)benzyl)carbamate

Compound tert-butyl (2,6-difluoro-4-(4,4,5,5-tetramethyl-1,3,2-dioxaborolan-2-yl)benzyl)carbamate was prepared from tert-butyl (4-bromo-2,6-difluorobenzyl)carbamate (1.70 g, 5.28 mmol) following a similar procedure to that described for the synthesis of tert-butyl (3-cyclopropyl-4-(4,4,5,5-tetramethyl-1,3,2-dioxaborolan-2-yl)benzyl)carbamate, and was isolated as a brown solid.

Yield 1.75 g (90%). ¹H NMR (400 MHz, DMSO) δ 7.29 (t, J=5.6 Hz, 1H), 7.20 (d, J=7.2 Hz, 2H), 4.20 (d, J=5.6 Hz, 2H), 1.36 (s, 9H), 1.30 (s, 12H). m/z: [ESI⁺] 314 (M+H−56)⁺.

### tert-butyl (2,6-difluoro-4-(7-((3-(piperidin-1-yl)propyl)carbamoyl)benzo[d]imidazo[2,1-b]thiazol-2-yl)benzyl)carbamate

Compound tert-butyl (2,6-difluoro-4-(7-((3-(piperidin-1-yl)propyl)carbamoyl)benzo[d]imidazo[2,1-b]thiazol-2-yl)benzyl)carbamate was prepared from 2-bromo-N-(3-(piperidin-1-yl)propyl)benzo[d]imidazo[2,1-b]thiazole-7-carboxamide (500 mg, 1.187 mmol) and tert-butyl (2,6-difluoro-4-(4,4,5,5-tetramethyl-1,3,2-dioxaborolan-2-yl)benzyl)carbamate (876 mg, 2.373 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a light yellow solid.

Yield 278 mg (40%). ¹H NMR (400 MHz, DMSO) δ 8.96 (s, 1H), 8.68 (t, J=5.6 Hz, 1H), 8.51 (d, J=1.6 Hz, 1H), 8.04 (dd, J=1.6, 8.4 Hz, 1H), 7.99 (d, J=8.4 Hz, 1H), 7.51 (d, J=8.0 Hz, 2H), 7.31 (t, J=5.6 Hz, 1H), 4.21 (d, J=5.6 Hz, 2H), 3.38-3.29 (m, 2H), 2.48-2.39 (m, 6H), 1.81-1.67 (m, 2H), 1.61-1.49 (m, 4H), 1.38 (s, 9H), 1.47-1.36 (m, 2H). M/z: [ESI⁺] 584 (M+H)⁺.

### tert-butyl (2-methyl-4-(7-((3-(piperidin-1-yl)propyl)carbamoyl)benzo[d]imidazo[2,1-b]thiazol-2-yl)benzyl)carbamate

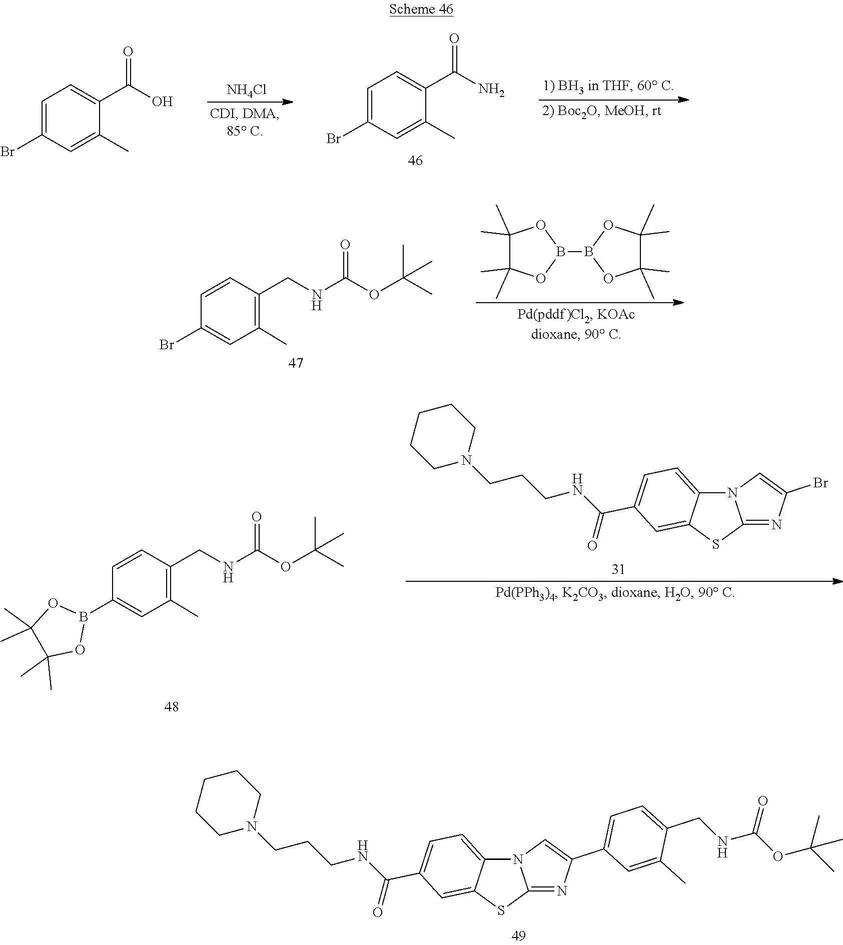

### 4-bromo-2-methylbenzamide

Compound 4-bromo-2-methylbenzamide was prepared from 4-bromo-2-methylbenzoic acid (4.50 g, 20.93 mmol) and ammonium chloride (2.24 g, 41.85 mmol) following a similar procedure to that described for the synthesis of 4-bromo-3-cyclopropylbenzamide, and was isolated as an off-white solid.

Yield 3.30 g (74%). ¹H NMR (400 MHz, CDCl₃) δ 7.44 (d, J=2.0 Hz, 1H), 7.38 (dd, J=2.0, 8.0 Hz, 1H), 7.34 (d, J=8.0 Hz, 1H), 5.76 (br s, 2H), 2.50 (s, 3H). m/z: [ESI⁺] 214, 216 (M+H)⁺.

### tert-butyl (4-bromo-2-methylbenzyl)carbamate

Compound tert-butyl (4-bromo-2-methylbenzyl)carbamate was prepared from 4-bromo-2-methylbenzamide (3.30 g, 15.42 mmol) following a similar procedure to that described for the synthesis of tert-butyl (4-bromo-3-cyclopropylbenzyl)carbamate, and was isolated as an off-white solid.

Yield 1.40 g (30%). ¹H NMR (400 MHz, CDCl₃) δ 7.33 (d, J=2.0 Hz, 1H), 7.30 (dd, J=2.0, 8.0 Hz, 1H), 7.13 (d, J=8.0 Hz, 1H), 4.71 (t, J=5.6 Hz, 1H), 4.28 (d, J=5.6 Hz, 2H), 2.32 (s, 3H), 1.48 (s, 9H). m/z: [ESi⁺] 244, 246 (M+H−56)⁺.

### tert-butyl (2-methyl-4-(4,4,5,5-tetramethyl-1,3,2-dioxaborolan-2-yl)benzyl)carbamate

Compound tert-butyl (2-methyl-4-(4,4,5,5-tetramethyl-1,3,2-dioxaborolan-2-yl)benzyl)carbamate was prepared from tert-butyl (4-bromo-2-methylbenzyl)carbamate (1.40 g, 4.66 mmol) following a similar procedure to that described for the synthesis of tert-butyl (3-cyclopropyl-4-(4,4,5,5-tetramethyl-1,3,2-dioxaborolan-2-yl)benzyl)carbamate. The reaction solution was used in the next step directly without further purification. m/z: [ESI⁺] 292 (M+H−56)⁺.

### tert-butyl (2-methyl-4-(7-((3-(piperidin-1-yl)propyl)carbamoyl)benzo[d]imidazo[2,1-b]thiazol-2-yl)benzyl)carbamate

Compound tert-butyl (2-methyl-4-(7-((3-(piperidin-1-yl)propyl)carbamoyl)benzo[d]imidazo[2,1-b]thiazol-2-yl)benzyl)carbamate was prepared from 2-bromo-N-(3-(piperidin-1-yl)propyl)benzo[d]imidazo[2,1-b]thiazole-7-carboxamide (523 mg, 1.241 mmol) and tert-butyl (2-methyl-4-(4,4,5,5-tetramethyl-1,3,2-dioxaborolan-2-yl)benzyl)carbamate (crude solution) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a brown solid.

Yield 251 mg (36%). M/z: [ESI⁺] 562 (M+H)⁺.

### tert-butyl (2-(difluoromethyl)-4-(7-((3-(piperidin-1-yl)propyl)carbamoyl)benzo[d]imidazo[2,1-b]thiazol-2-yl)benzyl)carbamate

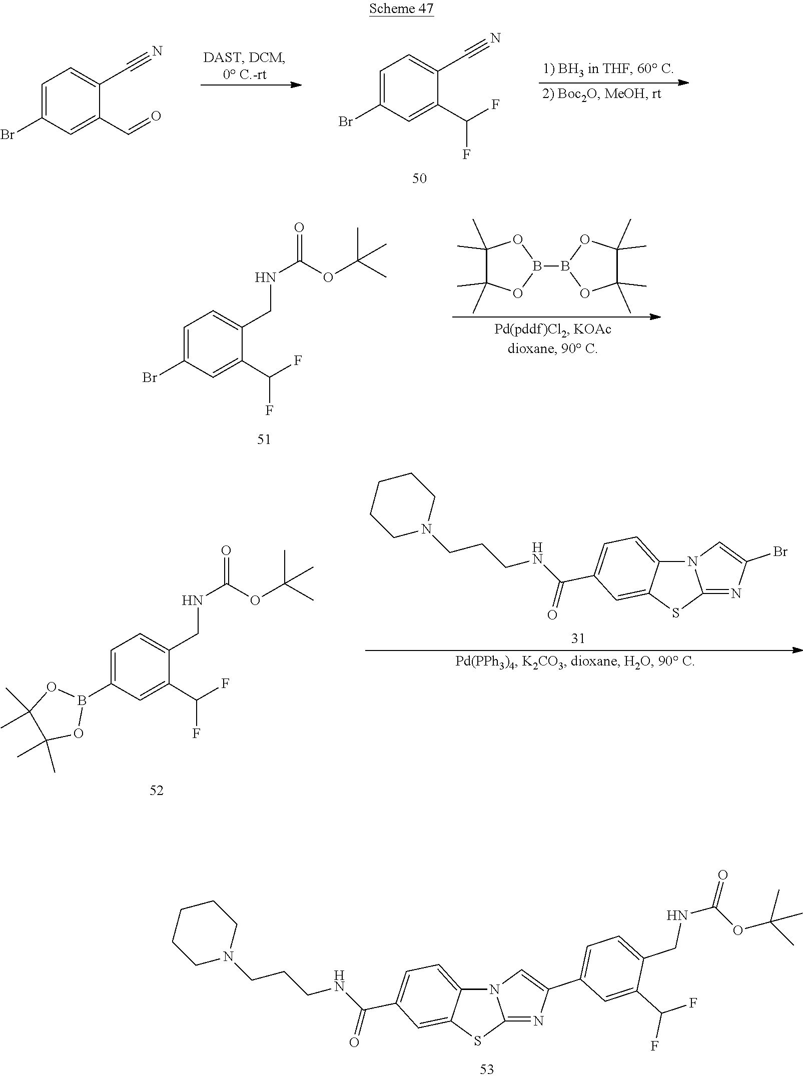

### 4-bromo-2-(difluoromethyl)benzonitrile

To a stirred solution of 4-bromo-2-formylbenzonitrile (1.00 g, 4.76 mmol) in dichloromethane (20 mL) was added diethylaminosulfur trifluoride (1.15 g, 7.14 mmol) drop-wise at 0° C. under a nitrogen atmosphere. The resulting solution was stirred for 1 h at room temperature under a nitrogen atmosphere. The reaction was quenched with saturated aqueous ammonium chloride (50 mL) at 0° C. The resulting mixture was extracted with dichloromethane (3×50 mL). The combined organic layers were washed with brine (50 mL) and dried over anhydrous sodium sulfate. After filtration, the filtrate was concentrated under reduced pressure. The residue was purified by silica gel column chromatography, eluting with 1%-20% ethyl acetate in petroleum ether to afford 4-bromo-2-(difluoromethyl)benzonitrile as a light yellow oil.

Yield 0.90 g (81%). ¹H NMR (400 MHz, CDCl₃) δ 7.94 (s, 1H), 7.82-7.75 (m, 1H), 7.68-7.62 (m, 1H), 6.91 (t, J=54.4 Hz, 1H). ¹⁹F NMR (376 MHz, CDCl₃) δ −111.29. No MS signal.

### tert-butyl (4-bromo-2-(difluoromethyl)benzyl)carbamate

Compound tert-butyl (4-bromo-2-(difluoromethyl)benzyl)carbamate was prepared from 4-bromo-2-(difluoromethyl)benzonitrile (0.90 g, 3.88 mmol) following a similar procedure to that described for the synthesis of tert-butyl (4-bromo-3-cyclopropylbenzyl)carbamate, and was isolated as a light yellow oil.

Yield 0.80 g (61%). ¹H NMR (400 MHz, CDCl₃) δ 7.69 (d, J=2.0 Hz, 1H), 7.61 (dd, J=2.0, 8.0 Hz, 1H), 7.35 (d, J=8.0 Hz, 1H), 6.84 (t, J=55.2 Hz, 1H), 4.89 (br s, 1H), 4.41 (d, J=6.0 Hz, 2H), 1.47 (s, 9H). ¹⁹F NMR (376 MHz, CDCl₃) δ −112.81. m/z: [ESi⁺] 280, 282 (M+H−56)⁺.

### tert-butyl (2-(difluoromethyl)-4-(4,4,5,5-tetramethyl-1,3,2-dioxaborolan-2-yl)benzyl)carbamate

Compound tert-butyl (2-(difluoromethyl)-4-(4,4,5,5-tetramethyl-1,3,2-dioxaborolan-2-yl)benzyl)carbamate was prepared from tert-butyl (4-bromo-2-(difluoromethyl)benzyl)carbamate (300 mg, 0.892 mmol) following a similar procedure to that described for the synthesis of tert-butyl (3-cyclopropyl-4-(4,4,5,5-tetramethyl-1,3,2-dioxaborolan-2-yl)benzyl)carbamate, and was isolated as a light yellow oil.

Yield 200 mg (58%). ¹H NMR (400 MHz, CDCl₃) δ. 7.93 (s, 1H), 7.90 (d, J=8.0 Hz, 1H), 7.48 (d, J=8.0 Hz, 1H), 6.83 (t, J=55.2 Hz, 1H), 4.92 (br s, 1H), 4.50 (d, J=6.0 Hz, 2H), 1.46 (s, 9H), 1.36 (s, 12H). m/z: [ESI⁺] 328 (M+H−56)⁺.

### tert-butyl (2-(difluoromethyl)-4-(7-((3-(piperidin-1-yl)propyl)carbamoyl)benzo[d]imidazo[2,1-b]thiazol-2-yl)benzyl)carbamate

Compound tert-butyl (2-(difluoromethyl)-4-(7-((3-(piperidin-1-yl)propyl)carbamoyl)benzo[d]imidazo[2,1-b]thiazol-2-yl)benzyl)carbamate was prepared from 2-bromo-N-(3-(piperidin-1-yl)propyl)benzo[d]imidazo[2,1-b]thiazole-7-carboxamide (150 mg, 0.356 mmol) and tert-butyl (2-(difluoromethyl)-4-(4,4,5,5-tetramethyl-1,3,2-dioxaborolan-2-yl)benzyl)carbamate (200 mg, 0.522 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a white solid.

Yield 100 mg (47%). ¹H NMR (400 MHz, DMSO) δ 8.93 (s, 1H), 8.65 (t, J=5.6 Hz, 1H), 8.49 (d, J=1.6 Hz, 1H), 8.10-7.97 (m, 4H), 7.52-7.43 (m, 2H), 7.32 (t, J=55.2 Hz, 1H), 4.31 (d, J=6.0 Hz, 2H), 2.54-2.52 (m, 2H), 2.44-2.37 (m, 6H), 1.77-1.68 (m, 2H), 1.58-1.48 (m, 4H), 1.42 (s, 9H), 1.47-1.36 (m, 2H). ¹⁹F NMR (376 MHz, DMSO) δ −111.73. M/z: [ESI⁺] 598 (M+H)⁺.

### tert-butyl (3-(difluoromethyl)-4-(7-((3-(piperidin-1-yl)propyl)carbamoyl)benzo[d]imidazo[2,1-b]thiazol-2-yl)benzyl)carbamate

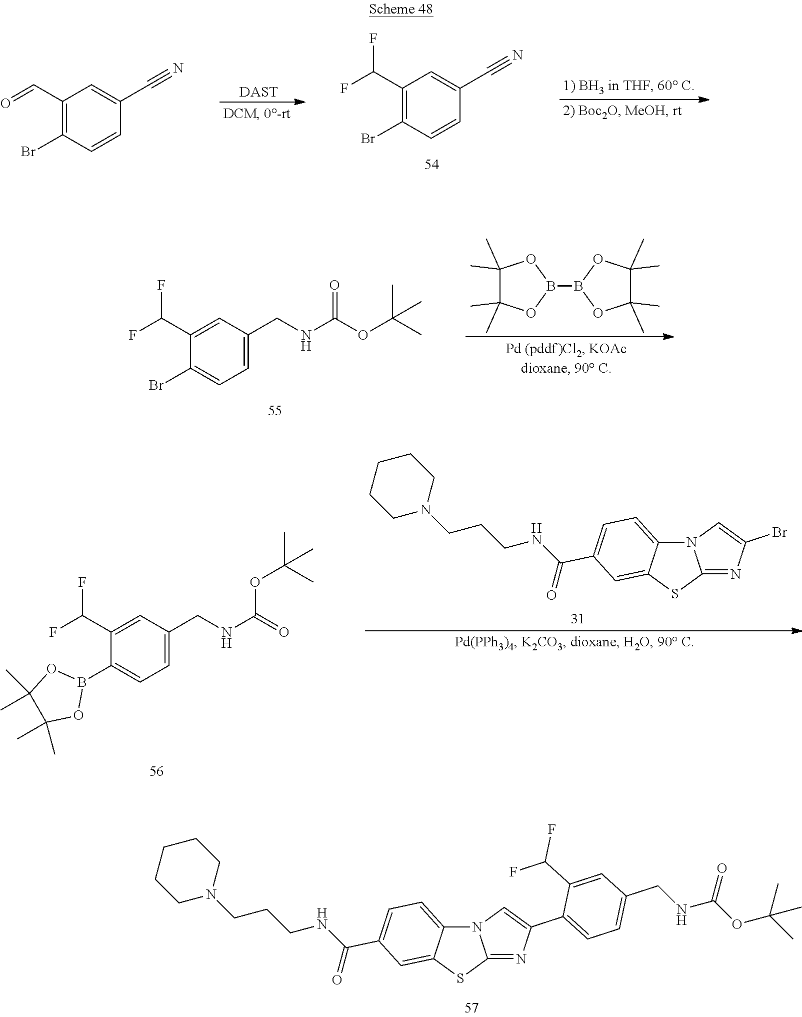

### 4-bromo-3-(difluoromethyl)benzonitrile

Compound 4-bromo-3-(difluoromethyl)benzonitrile was prepared from 4-bromo-3-formylbenzonitrile (2.00 g, 9.52 mmol) following a similar procedure to that described for the synthesis of 4-bromo-2-(difluoromethyl)benzonitrile, and was isolated as an off-white solid.

Yield 1.50 g (68%). ¹H NMR (400 MHz, CDCl₃) δ 7.96 (d, J=2.0 Hz, 1H), 7.79 (d, J=8.4 Hz, 1H), 7.64 (dd, J=2.0, 8.4 Hz, 1H), 6.91 (t, J=54.4 Hz, 1H). No MS signal.

### tert-butyl (4-bromo-3-(difluoromethyl)benzyl)carbamate

Compound tert-butyl (4-bromo-3-(difluoromethyl)benzyl)carbamate was prepared from 4-bromo-3-(difluoromethyl)benzonitrile (1.00 g, 4.31 mmol) following a similar procedure to that described for the synthesis of tert-butyl (4-bromo-3-cyclopropylbenzyl)carbamate, and was isolated as an off-white solid.

Yield 741 mg (51%). ¹H NMR (400 MHz, CD₃OD) δ. 7.64 (d, J=8.4 Hz, 1H), 7.61 (d, J=2.0 Hz, 1H), 7.35 (dd, J=2.0, 8.4 Hz, 1H), 6.98 (t, J=54.4 Hz, 1H), 4.26 (s, 2H), 1.47 (s, 9H). NH proton not observed. m/z: [ESI⁻] 334, 336 (M−H)⁻.

### tert-butyl (3-(difluoromethyl)-4-(4,4,5,5-tetramethyl-1,3,2-dioxaborolan-2-yl)benzyl)carbamate

Compound tert-butyl (3-(difluoromethyl)-4-(4,4,5,5-tetramethyl-1,3,2-dioxaborolan-2-yl)benzyl)carbamate was prepared from tert-butyl (4-bromo-3-(difluoromethyl)benzyl)carbamate (700 mg, 2.082 mmol) following a similar procedure to that described for the synthesis of tert-butyl (3-cyclopropyl-4-(4,4,5,5-tetramethyl-1,3,2-dioxaborolan-2-yl)benzyl)carbamate, and was isolated as a brown oil.

Yield 632 mg (79%). ¹H NMR (400 MHz, CDCl₃) δ 7.87 (d, J=8.0 Hz, 1H), 7.64 (s, 1H), 7.40 (d, J=8.0 Hz, 1H), 7.38 (t, J=56.0 Hz, 1H), 4.89 (br s, 1H), 4.40 (d, J=6.0 Hz, 2H), 1.49 (s, 9H), 1.37 (s, 12H). m/z: [ESI⁺] 328 (M+H−56)⁺.

### tert-butyl (3-(difluoromethyl)-4-(7-((3-(piperidin-1-yl)propyl)carbamoyl)benzo[d]imidazo[2,1-b]thiazol-2-yl)benzyl)carbamate

Compound tert-butyl (3-(difluoromethyl)-4-(7-((3-(piperidin-1-yl)propyl)carbamoyl)benzo[d]imidazo[2,1-b]thiazol-2-yl)benzyl)carbamate was prepared from 2-bromo-N-(3-(piperidin-1-yl)propyl)benzo[d]imidazo[2,1-b]thiazole-7-carboxamide (300 mg, 0.712 mmol) and tert-butyl (3-(difluoromethyl)-4-(4,4,5,5-tetramethyl-1,3,2-dioxaborolan-2-yl)benzyl)carbamate (409 mg, 1.068 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a yellow solid.

Yield 200 mg (47%). ¹H NMR (400 MHz, DMSO) δ 8.95 (t, J=5.6 Hz, 1H), 8.78 (d, J=1.6 Hz, 1H), 8.61 (s, 1H), 8.57-8.50 (m, 1H), 8.19 (d, J=8.4 Hz, 1H), 8.13 (dd, J=1.6, 8.4 Hz, 1H), 7.94 (d, J=2.0 Hz, 1H), 7.89 (d, J=8.0 Hz, 1H), 7.78 (t, J=54.8 Hz, 1H), 7.81-7.76 (m, 1H), 4.21-4.11 (m, 2H), 3.50-3.34 (m, 4H), 3.14-3.03 (m, 2H), 2.95-2.79 (m, 2H), 2.06-1.97 (m, 2H), 1.89-1.67 (m, 6H), 1.07 (s, 9H). M/z: [ESI⁺] 598 (M+H)⁺.

### tert-butyl (2-chloro-4-(7-((3-(piperidin-1-yl)propyl)carbamoyl)benzo[d]imidazo[2,1-b]thiazol-2-yl)benzyl)carbamate

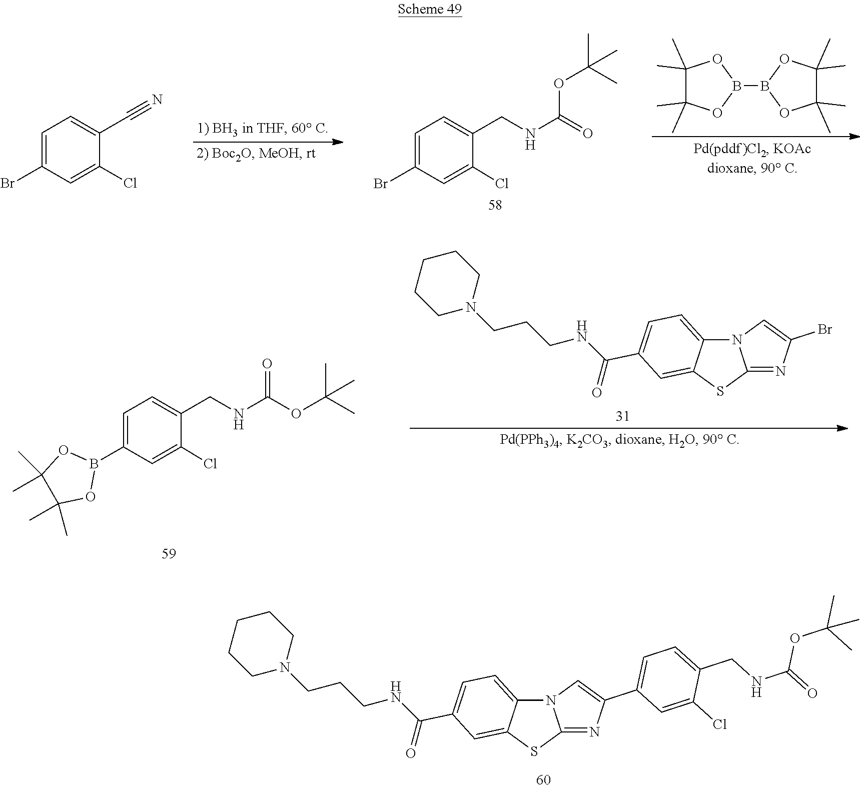

### tert-butyl (4-bromo-2-chlorobenzyl)carbamate

Compound tert-butyl (4-bromo-2-chlorobenzyl)carbamate was prepared from 4-bromo-2-chlorobenzonitrile (3.00 g, 13.86 mmol) following a similar procedure to that described for the synthesis of tert-butyl (4-bromo-3-cyclopropylbenzyl)carbamate, and was isolated as a white solid.

Yield 3.40 g (77%). ¹H NMR (400 MHz, CDCl₃) δ 7.54 (d, J=2.0 Hz, 1H), 7.40 (dd, J=2.0, 8.0 Hz, 1H), 7.30-7.26 (m, 1H), 5.00 (s, 1H), 4.36 (d, J=6.4 Hz, 2H), 1.47 (s, 9H). m/z: [ESI⁺] 264, 266, 268 (M+H−56)⁺.

### tert-butyl (2-chloro-4-(4,4,5,5-tetramethyl-1,3,2-dioxaborolan-2-yl)benzyl)carbamate

Compound tert-butyl (2-chloro-4-(4,4,5,5-tetramethyl-1,3,2-dioxaborolan-2-yl)benzyl)carbamate was prepared from tert-butyl (4-bromo-2-chlorobenzyl)carbamate (3.40 g, 10.61 mmol) following a similar procedure to that described for the synthesis of tert-butyl (3-cyclopropyl-4-(4,4,5,5-tetramethyl-1,3,2-dioxaborolan-2-yl)benzyl)carbamate, and was isolated as a brown solid.

Yield 3.19 g (82%). ¹H NMR (400 MHz, CDCl₃) δ 7.80 (d, J=1.2 Hz, 1H), 7.68 (dd, J=1.2, 7.6 Hz, 1H), 7.40 (d, J=7.6 Hz, 1H), 5.00 (br s, 1H), 4.43 (d, J=6.0 Hz, 2H), 1.47 (s, 9H), 1.36 (s, 12H). m/z: [ESi⁺] 312, 314 (M+H−56)⁺.

### tert-butyl (2-chloro-4-(7-((3-(piperidin-1-yl)propyl)carbamoyl)benzo[d]imidazo[2,1-b]thiazol-2-yl)benzyl)carbamate

Compound tert-butyl (2-chloro-4-(7-((3-(piperidin-1-yl)propyl)carbamoyl)benzo[d]imidazo[2,1-b]thiazol-2-yl)benzyl)carbamate was prepared from 2-bromo-N-(3-(piperidin-1-yl)propyl)benzo[d]imidazo[2,1-b]thiazole-7-carboxamide (200 mg, 0.475 mmol) and tert-butyl (2-chloro-4-(4,4,5,5-tetramethyl-1,3,2-dioxaborolan-2-yl)benzyl)carbamate (262 mg, 0.713 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a light yellow solid.

Yield 139 mg (50%). ¹H NMR (400 MHz, DMSO) δ 8.90 (s, 1H), 8.65 (t, J=5.6 Hz, 1H), 8.49 (s, 1H), 8.05-8.01 (m, 2H), 7.90 (d, J=1.6 Hz, 1H), 7.83 (d, J=8.0 Hz, 1H), 7.44 (t, J=6.0 Hz, 1H), 7.39 (d, J=8.0 Hz, 1H), 4.24 (d, J=6.0 Hz, 2H), 3.37-3.30 (m, 2H), 2.52-2.40 (m, 6H), 1.80-1.69 (m, 2H), 1.60-1.49 (m, 4H), 1.43 (s, 9H), 1.47-1.36 (m, 2H). m/z: [ESI⁺] 582, 584 (M+H)⁺.

### tert-butyl (4-(7-((3-(piperidin-1-yl)propyl)carbamoyl)benzo[d]imidazo[2,1-b]thiazol-2-yl)-2-(trifluoromethyl)benzyl)carbamate

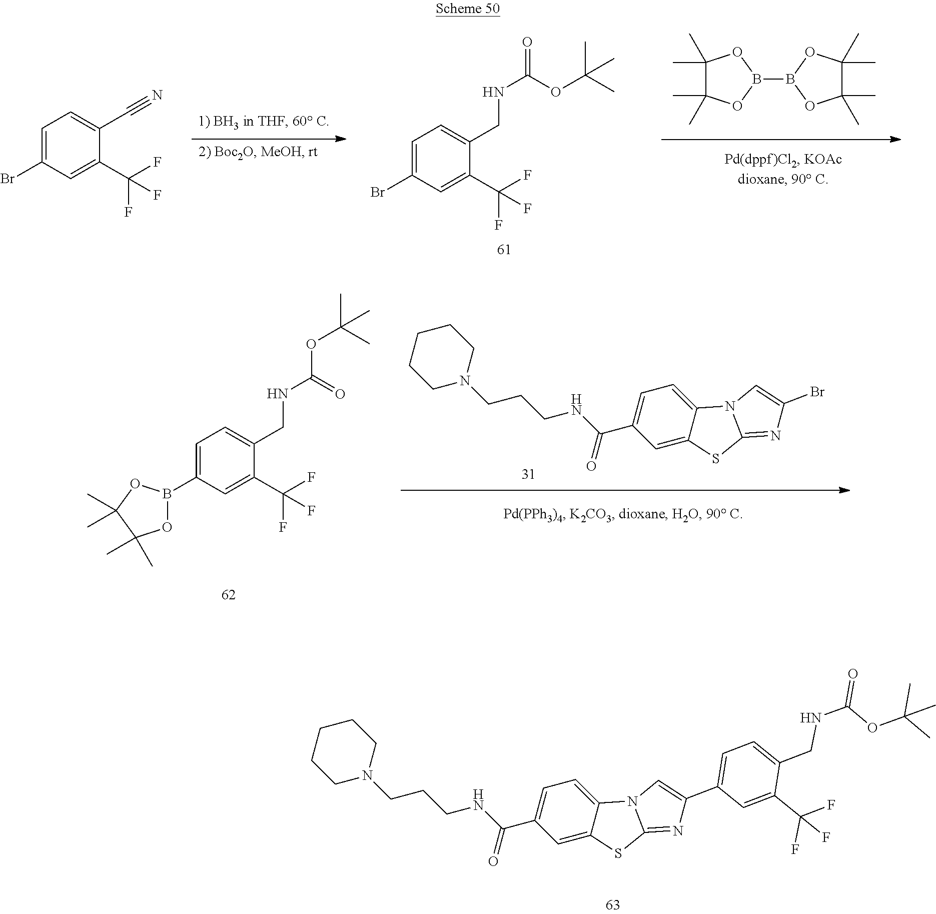

### tert-butyl (4-bromo-2-(trifluoromethyl)benzyl)carbamate

Compound tert-butyl (4-bromo-2-(trifluoromethyl)benzyl)carbamate was prepared from 4-bromo-2-(trifluoromethyl)benzonitrile (4.00 g, 16.00 mmol) following a similar procedure to that described for the synthesis of tert-butyl (4-bromo-3-cyclopropylbenzyl)carbamate, and was isolated as a brown solid.

Yield 4.50 g (79%). ¹H NMR (400 MHz, CDCl₃) δ 7.79 (d, J=2.0 Hz, 1H), 7.68 (dd, J=2.0, 8.4 Hz, 1H), 7.49 (d, J=8.4 Hz, 1H), 4.93 (br s, 1H), 4.46 (d, J=6.4 Hz, 2H), 1.47 (s, 9H). m/z: [ESI⁻] 352, 354 (M−H)⁻.

### tert-butyl (4-(4,4,5,5-tetramethyl-1,3,2-dioxaborolan-2-yl)-2-(trifluoromethyl)benzyl)carbamate

Compound tert-butyl (4-(4,4,5,5-tetramethyl-1,3,2-dioxaborolan-2-yl)-2-(trifluoromethyl)benzyl)carbamate was prepared from tert-butyl (4-bromo-2-(trifluoromethyl)benzyl)carbamate (4.50 g, 12.71 mmol) following a similar procedure to that described for the synthesis of tert-butyl (3-cyclopropyl-4-(4,4,5,5-tetramethyl-1,3,2-dioxaborolan-2-yl)benzyl)carbamate, and was isolated as a brown solid.

Yield 4.16 g (82%). ¹H NMR (400 MHz, CDCl₃) δ 8.08 (s, 1H), 7.97 (d, J=7.6 Hz, 1H), 7.59 (d, J=7.6 Hz, 1H), 4.92 (br s, 1H), 4.53 (d, J=6.4 Hz, 2H), 1.47 (s, 9H), 1.37 (s, 12H). m/z: [ESI⁺] 346 (M+H−56)⁺.

### tert-butyl (4-(7-((3-(piperidin-1-yl)propyl)carbamoyl)benzo[d]imidazo[2,1-b]thiazol-2-yl)-2-(trifluoromethyl)benzyl)carbamate

Compound tert-butyl (4-(7-((3-(piperidin-1-yl)propyl)carbamoyl)benzo[d]imidazo[2,1-b]thiazol-2-yl)-2-(trifluoromethyl)benzyl)carbamate was prepared from 2-bromo-N-(3-(piperidin-1-yl)propyl)benzo[d]imidazo[2,1-b]thiazole-7-carboxamide (200 mg, 0.475 mmol) and tert-butyl (4-(4,4,5,5-tetramethyl-1,3,2-dioxaborolan-2-yl)-2-(trifluoromethyl)benzyl)carbamate (286 mg, 0.713 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a brown solid.

Yield 160 mg (55%). ¹H NMR (400 MHz, DMSO) δ 8.99 (s, 1H), 8.67 (t, J=5.6 Hz, 1H), 8.50 (d, J=1.2 Hz, 1H), 8.17 (d, J=1.6 Hz, 1H), 8.14 (d, J=8.4 Hz, 1H), 8.05-8.03 (m, 2H), 7.58 (d, J=8.0 Hz, 1H), 7.54 (t, J=6.0 Hz, 1H), 4.36 (d, J=6.0 Hz, 2H), 3.37-3.29 (m, 2H), 2.42-2.32 (m, 6H), 1.78-1.67 (m, 2H), 1.59-1.48 (m, 4H), 1.43 (s, 9H), 1.45-1.35 (m, 2H). M/z: [ESI⁺] 616 (M+H)⁺.

### tert-butyl (3-chloro-4-(7-((3-(piperidin-1-yl)propyl)carbamoyl)benzo[d]imidazo[2,1-b]thiazol-2-yl)benzyl)carbamate

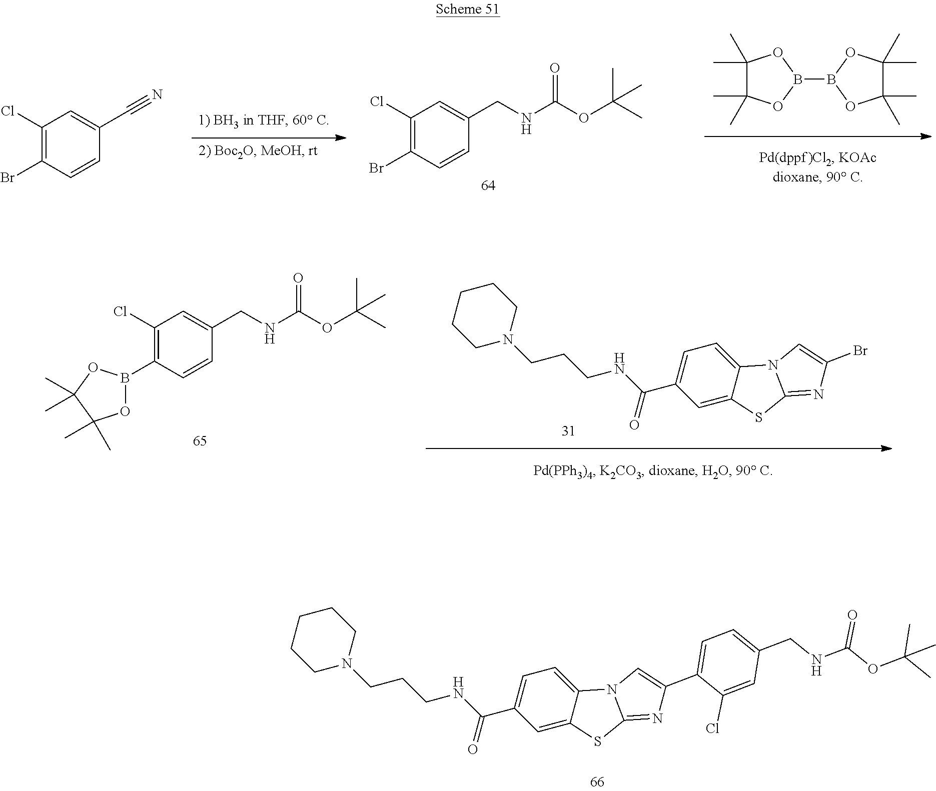

### tert-butyl (4-bromo-3-chlorobenzyl)carbamate

Compound tert-butyl (4-bromo-3-chlorobenzyl)carbamate was prepared from 4-bromo-3-chlorobenzonitrile (5.10 g, 23.56 mmol) following a similar procedure to that described for the synthesis of tert-butyl (4-bromo-3-cyclopropylbenzyl)carbamate, and was isolated as an off-white solid.

Yield 5.40 g (71%). ¹H NMR (400 MHz, CDCl₃) δ 7.58 (d, J=8.0 Hz, 1H), 7.39 (d, J=2.0 Hz, 1H), 7.06 (dd, J=2.0, 8.0 Hz, 1H), 4.92 (br s, 1H), 4.27 (d, J=6.0 Hz, 2H), 1.48 (s, 9H). m/z: [ESI⁻] 318, 320, 322 (M−H)⁻.

### tert-butyl (3-chloro-4-(4,4,5,5-tetramethyl-1,3,2-dioxaborolan-2-yl)benzyl)carbamate

Compound tert-butyl (3-chloro-4-(4,4,5,5-tetramethyl-1,3,2-dioxaborolan-2-yl)benzyl)carbamate was prepared from tert-butyl (4-bromo-3-chlorobenzyl)carbamate (1.00 g, 3.12 mmol) following a similar procedure to that described for the synthesis of tert-butyl (3-cyclopropyl-4-(4,4,5,5-tetramethyl-1,3,2-dioxaborolan-2-yl)benzyl)carbamate. The reaction solution was used in the next step directly without further purification. m/z: [ESi⁺] 312, 314 (M+H−56)⁺.

### tert-butyl (3-chloro-4-(7-((3-(piperidin-1-yl)propyl)carbamoyl)benzo[d]imidazo[2,1-b]thiazol-2-yl)benzyl)carbamate

Compound tert-butyl (3-chloro-4-(7-((3-(piperidin-1-yl)propyl)carbamoyl)benzo[d]imidazo[2,1-b]thiazol-2-yl)benzyl)carbamate was prepared from 2-bromo-N-(3-(piperidin-1-yl)propyl)benzo[d]imidazo[2,1-b]thiazole-7-carboxamide (0.57 g, 1.35 mmol) and tert-butyl (3-chloro-4-(4,4,5,5-tetramethyl-1,3,2-dioxaborolan-2-yl)benzyl)carbamate (crude reaction solution) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a brown solid.

Yield 0.23 g (29%). ¹H NMR (400 MHz, CDCl₃) δ 8.52-8.46 (m, 1H), 8.44 (s, 1H), 8.41-8.34 (m, 1H), 8.25-8.17 (m, 2H), 7.73 (d, J=8.4 Hz, 1H), 7.41 (s, 1H), 7.32-7.28 (m, 1H), 4.95 (br s, 1H), 4.40-4.31 (m, 2H), 3.71-3.59 (m, 2H), 3.25-3.04 (m, 2H), 2.72-2.58 (m, 4H), 2.26-2.12 (m, 2H), 2.06-1.86 (m, 6H), 1.50 (s, 9H). m/z: [ESI⁺] 582, 584 (M+H)⁺.

### tert-butyl (3-fluoro-4-(7-((3-(piperidin-1-yl)propyl)carbamoyl)benzo[d]imidazo[2,1-b]thiazol-2-yl)benzyl)carbamate

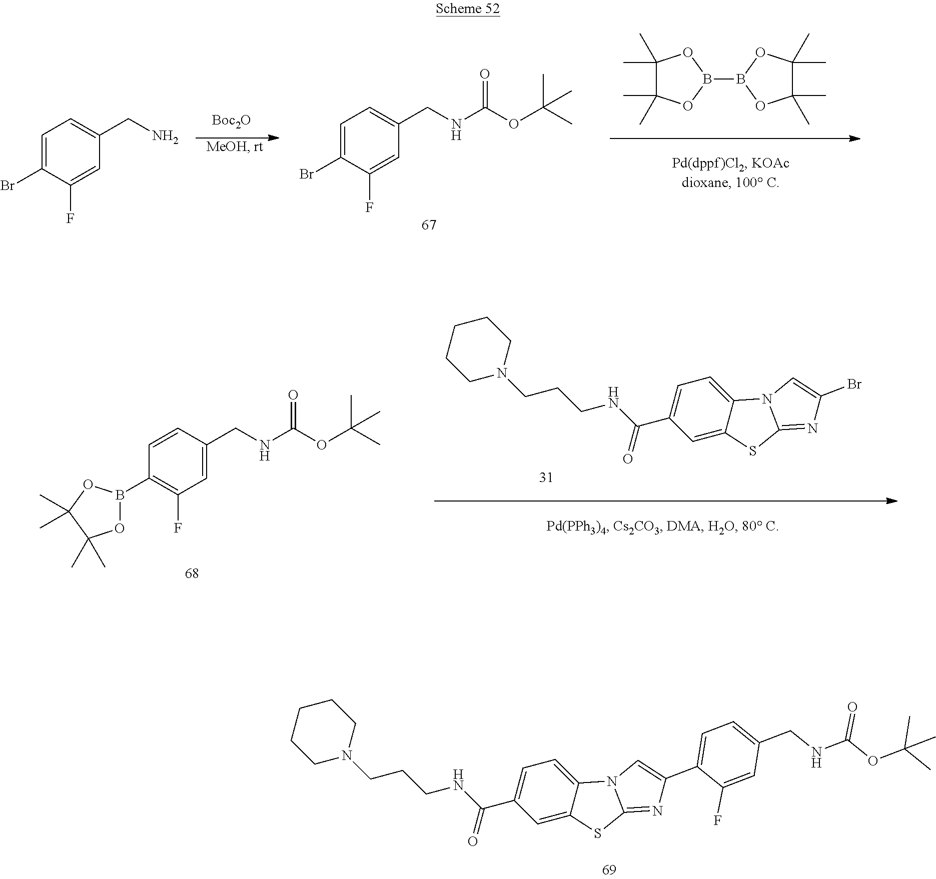

### tert-butyl (4-bromo-3-fluorobenzyl)carbamate

To a stirred solution of (4-bromo-3-fluorophenyl)methanamine (2.00 g, 9.80 mmol) in methanol (20 mL) was added di-tert-butyl dicarbonate (4.28 g, 19.61 mmol) at room temperature. The resulting solution was stirred for 16 h at room temperature. The precipitated solids were collected by filtration and the filter cake was washed with water (10 mL) and dried in a vacuum oven to afford tert-butyl (4-bromo-3-fluorobenzyl)carbamate as a white solid.

Yield 2.95 g (99%). ¹H NMR (400 MHz, CDCl₃) δ 7.51 (dd, J=7.2, 8.4 Hz, 1H), 7.08 (dd, J=2.0, 9.2 Hz, 1H), 6.97 (dd, J=2.0, 8.4 Hz, 1H), 4.93 (br s, 1H), 4.29 (d, J=4.8 Hz, 2H), 1.48 (s, 9H). m/z: [ESI⁻] 302, 304 (M−H)⁻.

### tert-butyl (3-fluoro-4-(4,4,5,5-tetramethyl-1,3,2-dioxaborolan-2-yl)benzyl)carbamate

Compound tert-butyl (3-fluoro-4-(4,4,5,5-tetramethyl-1,3,2-dioxaborolan-2-yl)benzyl)carbamate was prepared from tert-butyl (4-bromo-3-fluorobenzyl)carbamate (2.90 g, 9.53 mmol) following a similar procedure to that described for the synthesis of tert-butyl (3-cyclopropyl-4-(4,4,5,5-tetramethyl-1,3,2-dioxaborolan-2-yl)benzyl)carbamate, and was isolated as a brown solid.

Yield 0.77 g (23%). ¹H NMR (400 MHz, CDCl₃) δ 7.71 (dd, J=6.0, 7.6 Hz, 1H), 7.07 (d, J=7.6 Hz, 1H), 6.98 (d, J=10.0 Hz, 1H), 4.88 (br s, 1H), 4.34 (d, J=6.0 Hz, 2H), 1.48 (s, 9H), 1.38 (s, 12H). m/z: [ESI⁺] 296 (M+1-56)⁺.

### tert-butyl (3-fluoro-4-(7-((3-(piperidin-1-yl)propyl)carbamoyl)benzo[d]imidazo[2,1-b]thiazol-2-yl)benzyl)carbamate

Compound tert-butyl (3-fluoro-4-(7-((3-(piperidin-1-yl)propyl)carbamoyl)benzo[d]imidazo[2,1-b]thiazol-2-yl)benzyl)carbamate was prepared from tert-butyl (3-fluoro-4-(4,4,5,5-tetramethyl-1,3,2-dioxaborolan-2-yl)benzyl)carbamate (650 mg, 1.851 mmol) and 2-bromo-N-(3-(piperidin-1-yl)propyl)benzo[d]imidazo[2,1-b]thiazole-7-carboxamide (779 mg, 1.849 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a light brown solid.

Yield 300 mg (29%). ¹H NMR (400 MHz, CDCl₃) δ 8.48 (s, 1H), 8.26-8.21 (m, 2H), 8.18 (d, J=8.4 Hz, 1H), 8.03 (t, J=5.6 Hz, 1H), 7.86 (d, J=8.4 Hz, 1H), 7.24 (d, J=8.0 Hz, 1H), 7.19-7.14 (m, 1H), 5.13-4.98 (br s, 1H), 4.47-4.30 (m, 2H), 3.72-3.53 (m, 4H), 3.26-3.06 (m, 2H), 2.82-2.63 (m, 2H), 2.27-2.19 (m, 2H), 1.99-1.87 (m, 6H), 1.51 (s, 9H). M/z: [ESI⁺] 566 (M+H)⁺.

### tert-butyl 2-(3-fluoro-4-(7-((3-(piperidin-1-yl)propyl)carbamoyl)benzo[d]imidazo[2,1-b]thiazol-2-yl)phenyl)pyrrolidine-1-carboxylate

### tert-butyl (R)-2-(3-fluoro-4-(7-((3-(piperidin-1-yl)propyl)carbamoyl)benzo[d]imidazo[2,1-b]thiazol-2-yl)phenyl)pyrrolidine-1-carboxylate

### tert-butyl (S)-2-(3-fluoro-4-(7-((3-(piperidin-1-yl)propyl)carbamoyl)benzo[d]imidazo[2,1-b]thiazol-2-yl)phenyl)pyrrolidine-1-carboxylate

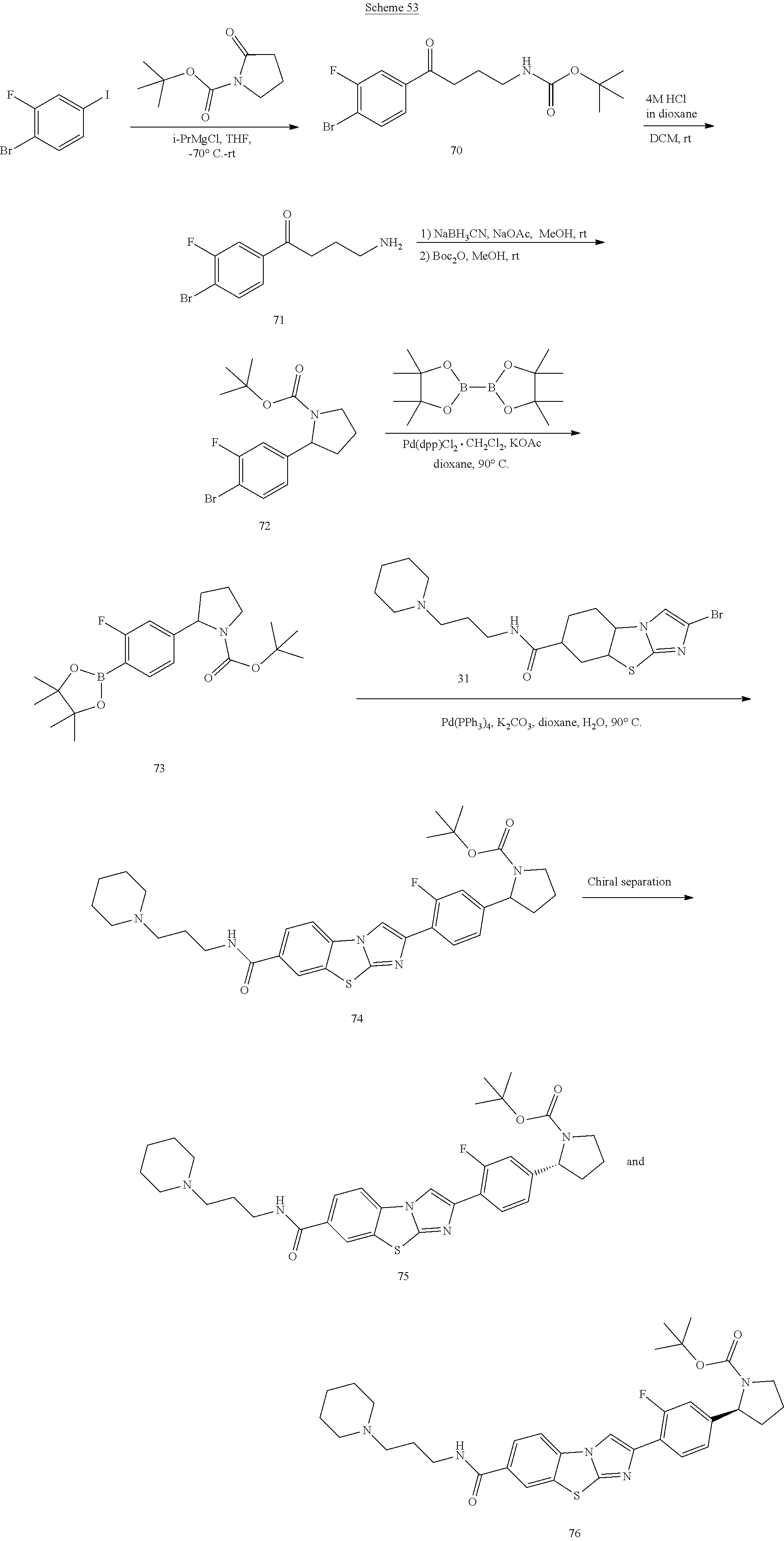

### tert-butyl (4-(4-bromo-3-fluorophenyl)-4-oxobutyl)carbamate

To a stirred solution of 1-bromo-2-fluoro-4-iodobenzene (46.00 g, 152.88 mmol) in tetrahydrofuran (400 mL) was added isopropylmagnesium chloride (2.0 M in tetrahydrofuran, 84 mL, 168.00 mmol) dropwise at 0° C. The resulting solution was stirred for 3 h at 0° C. under a nitrogen atmosphere. To the above solution was added tert-butyl 2-oxopyrrolidine-1-carboxylate (33.98 g, 183.46 mmol) in tetrahydrofuran (60 mL) dropwise over 10 min at −78° C. The resulting solution was stirred for additional 1 h at −78° C. The mixture was allowed to warm to room temperature and quenched with water (400 mL) at room temperature. The resulting mixture was extracted with ethyl acetate (3×400 mL). The combined organic layers were washed with brine (3×100 mL), dried over anhydrous sodium sulfate and concentrated under reduced pressure. The residue was purified by silica gel column chromatography, eluting with 1%-20% ethyl acetate in petroleum ether to afford tert-butyl (4-(4-bromo-3-fluorophenyl)-4-oxobutyl)carbamate as a white solid.

Yield 22.00 g (40%). ¹H NMR (400 MHz, CDCl₃) δ 7.74-7.56 (m, 3H), 4.69 (br s, 1H), 3.29-3.16 (m, 2H), 2.99 (t, J=7.2 Hz, 2H), 2.01-1.87 (m, 2H), 1.43 (s, 9H). m/z: [ESI⁺] 360, 362 (M+H)⁺.

### 4-amino-1-(4-bromo-3-fluorophenyl)butan-1-one hydrochloride

To a stirred solution of tert-butyl (4-(4-bromo-3-fluorophenyl)-4-oxobutyl)carbamate (8.53 g, 23.68 mmol) in dichloromethane (120 mL) was added a solution of 4.0 M solution of hydrogen chloride in dioxane (60 mL) dropwise at room temperature. The resulting mixture was stirred for 4 h at room temperature. The resulting mixture was filtered. The filtered cake was washed with petroleum ether (3×50 mL). The resulting solid was dried under vacuum to afford 4-amino-1-(4-bromo-3-fluorophenyl)butan-1-one hydrochloride as a light yellow solid.

Yield 6.10 g (87%). ¹H NMR (400 MHz, DMSO) δ 8.03 (br s, 3H, NH₃+), 7.97-7.85 (m, 2H), 7.75 (dd, J=2.0, 8.4 Hz, 1H), 3.21 (t, J=7.2 Hz, 2H), 2.91-2.80 (m, 2H), 1.96-1.85 (m, 2H). m/Z: [ESI⁺] 260, 262 (M+H)⁺.

### tert-butyl 2-(4-bromo-3-fluorophenyl)pyrrolidine-1-carboxylate

To a stirred solution of 4-amino-1-(4-bromo-3-fluorophenyl)butan-1-one hydrochloride (16.00 g, 53.95 mmol) in methanol (240 mL) were added sodium acetate trihydrate (22.02 g, 161.85 mmol) and sodium cyanoborohydride (6.78 g, 107.90 mmol) portion-wise at 0° C. The resulting solution was stirred for 16 h at room temperature. To the resulting solution was added di-tert-butyl dicarbonate (35.32 g, 161.83 mmol) at room temperature. The resulting solution was stirred for additional 16 h at room temperature. The resulting solution was concentrated under reduced pressure. The residue was purified by reverse phase flash chromatography with the following conditions: Column: Spherical C18, 20-40 m, 330 g; Mobile Phase A: water (plus 10 mM ammonium bicarbonate); Mobile Phase B: acetonitrile; Flow rate: 80 m/min; Gradient: 75%-95% B in 20 min; Detector: UV 254/220 nm. The fractions containing desired product were collected and concentrated under reduced pressure to afford tert-butyl 2-(4-bromo-3-fluorophenyl)pyrrolidine-1-carboxylate as a white solid.

Yield 17.75 g (96%). ¹H NMR (400 MHz, CDCl₃) δ 7.51-7.45 (m, 1H), 6.97 (dd, J=2.0, 9.6 Hz, 1H), 6.88 (dd, J=2.0, 8.4 Hz, 1H), 4.96-4.69 (m, 1H), 3.71-3.47 (m, 2H), 2.43-2.25 (m, 1H), 1.94-1.85 (m, 2H), 1.84-1.74 (m, 1H), 1.45 (s, 4H), 1.25 (s, 5H). m/z: [ESI⁺] 288, 290 (M+H−56)⁺.

### tert-butyl 2-(3-fluoro-4-(4,4,5,5-tetramethyl-1,3,2-dioxaborolan-2-yl)phenyl)pyrrolidine-1-carboxylate

Tert-butyl 2-(3-fluoro-4-(4,4,5,5-tetramethyl-1,3,2-dioxaborolan-2-yl)phenyl)pyrrolidine-1-carboxylate was prepared from tert-butyl 2-(4-bromo-3-fluorophenyl)pyrrolidine-1-carboxylate (2.00 g, 5.81 mmol) following a similar procedure to that described for the synthesis of tert-butyl (3-cyclopropyl-4-(4,4,5,5-tetramethyl-1,3,2-dioxaborolan-2-yl)benzyl)carbamate, and was isolated as a grey solid.

Yield 0.80 g (35%). ¹H NMR (400 MHz, CDCl₃) δ 7.71-7.65 (m, 1H), 6.97 (dd, J=1.6, 7.6 Hz, 1H), 6.86 (dd, J=1.6, 10.0 Hz, 1H), 5.01-4.74 (m, 1H), 3.67-3.55 (m, 2H), 2.39-2.28 (m, 1H), 1.97-1.75 (m, 3H), 1.47 (s, 3H), 1.37 (s, 12H), 1.25 (s, 6H). M/z: [ESI⁺] 336 (M+H−56)⁺.

### tert-butyl 2-(3-fluoro-4-(7-((3-(piperidin-1-yl)propyl)carbamoyl)benzo[d]imidazo[2,1-b]thiazol-2-yl)phenyl)pyrrolidine-1-carboxylate

Tert-butyl 2-(3-fluoro-4-(7-((3-(piperidin-1-yl)propyl)carbamoyl)benzo[d]imidazo[2,1-b]thiazol-2-yl)phenyl)pyrrolidine-1-carboxylate was prepared from 2-bromo-N-(3-(piperidin-1-yl)propyl)benzo[d]imidazo[2,1-b]thiazole-7-carboxamide (300 mg, 0.712 mmol) and tert-butyl 2-(3-fluoro-4-(4,4,5,5-tetramethyl-1,3,2-dioxaborolan-2-yl)phenyl)pyrrolidine-1-carboxylate (400 mg, 1.022 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a light yellow solid.

Yield 300 mg (70%). ¹H NMR (400 MHz, DMSO) δ 8.69 (d, J=3.6 Hz, 1H), 8.65 (t, J=5.6 Hz, 1H), 8.49 (d, J=1.6 Hz, 1H), 8.27 (d, J=8.4 Hz, 1H), 8.13-8.05 (m, 1H), 8.01 (dd, J=1.6, 8.4 Hz, 1H), 7.18-7.08 (m, 2H), 4.91-4.82 (m, 0.35H), 4.81-4.71 (m, 0.65H), 3.57-3.45 (m, 4H), 3.37-3.28 (m, 2H), 2.44-2.37 (m, 6H), 1.93-1.65 (m, 4H), 1.61-1.48 (m, 4H), 1.45-1.34 (m, 2H), 1.41 (s, 4H), 1.14 (s, 5H). m/z: [ESI⁺] 606 (M+H)⁺.

### tert-butyl (R)-2-(3-fluoro-4-(7-((3-(piperidin-1-yl)propyl)carbamoyl)benzo[d]imidazo[2,1-b]thiazol-2-yl)phenyl)pyrrolidine-1-carboxylate and tert-butyl (S)-2-(3-fluoro-4-(7-((3-(piperidin-1-yl)propyl)carbamoyl)benzo[d]imidazo[2,1-b]thiazol-2-yl)phenyl)pyrrolidine-1-carboxylate

Tert-butyl 2-(3-fluoro-4-(7-((3-(piperidin-1-yl)propyl)carbamoyl)benzo[d]imidazo[2,1-b]thiazol-2-yl)phenyl)pyrrolidine-1-carboxylate (300 mg, 0.495 mmol) was separated by chiral HPLC with the following conditions: (Column: CHIRALPAK IG, 2×25 cm, 5 μm; Mobile Phase A: Hexane (plus 0.5% 2 M NH₃-MeOH, v/v), Mobile Phase B: EtOH; Flow rate: 18 mL/min; Gradient: 50% B in 25 min; Detector: UV 254/220 nm; RT1 (min): 14.64; RT2 (min): 18.79). The faster eluting peak was concentrated under reduced pressure to afford tert-butyl (R)-2-(3-fluoro-4-(7-((3-(piperidin-1-yl)propyl)carbamoyl)benzo[d]imidazo[2,1-b]thiazol-2-yl)phenyl)pyrrolidine-1-carboxylate as a white solid;

Yield 130 mg (43%). ¹H NMR (400 MHz, CD₃OD) δ 8.53-8.45 (m, 1H), 8.39 (d, J=1.6 Hz, 1H), 8.15-7.94 (m, 3H), 7.15 (d, J=8.4 Hz, 1H), 7.11-7.00 (m, 1H), 4.98-4.85 (m, 1H), 3.50 (t, J=6.8 Hz, 2H), 3.40-3.36 (m, 4H), 2.84-2.60 (m, 6H), 2.50-2.36 (m, 1H), 2.03-1.83 (m, 3H), 1.78-1.68 (m, 4H), 1.63-1.54 (m, 2H), 1.49 (s, 3H), 1.25 (s, 6H). NH proton not observed. m/z: [ESI⁺] 606 (M+H)⁺.

The slower eluting peak was concentrated under reduced pressure to afford tert-butyl (S)-2-(3-fluoro-4-(7-((3-(piperidin-1-yl)propyl)carbamoyl)benzo[d]imidazo[2,1-b]thiazol-2-yl)phenyl)pyrrolidine-1-carboxylate as a white solid.

Yield 130 mg (43%). ¹H NMR (400 MHz, CD₃OD) δ 8.53-8.45 (m, 1H), 8.39 (d, J=1.6 Hz, 1H), 8.15-7.94 (m, 3H), 7.15 (d, J=8.4 Hz, 1H), 7.11-7.00 (m, 1H), 4.98-4.85 (m, 1H), 3.50 (t, J=6.8 Hz, 2H), 3.40-3.36 (m, 4H), 2.84-2.60 (m, 6H), 2.50-2.36 (m, 1H), 2.03-1.83 (m, 3H), 1.78-1.68 (m, 4H), 1.63-1.54 (m, 2H), 1.49 (s, 3H), 1.25 (s, 6H). NH proton not observed. m/z: [ESI⁺] 606 (M+H)⁺.

### tert-butyl (R)-2-(3-fluoro-4-(7-((3-(4-fluoropiperidin-1-yl)propyl)carbamoyl)benzo[d]imidazo[2,1-b]thiazol-2-yl)phenyl)pyrrolidine-1-carboxylate and tert-butyl (S)-2-(3-fluoro-4-(7-((3-(4-fluoropiperidin-1-yl)propyl)carbamoyl)benzo[d]imidazo[2,1-b]thiazol-2-yl)phenyl)pyrrolidine-1-carboxylate

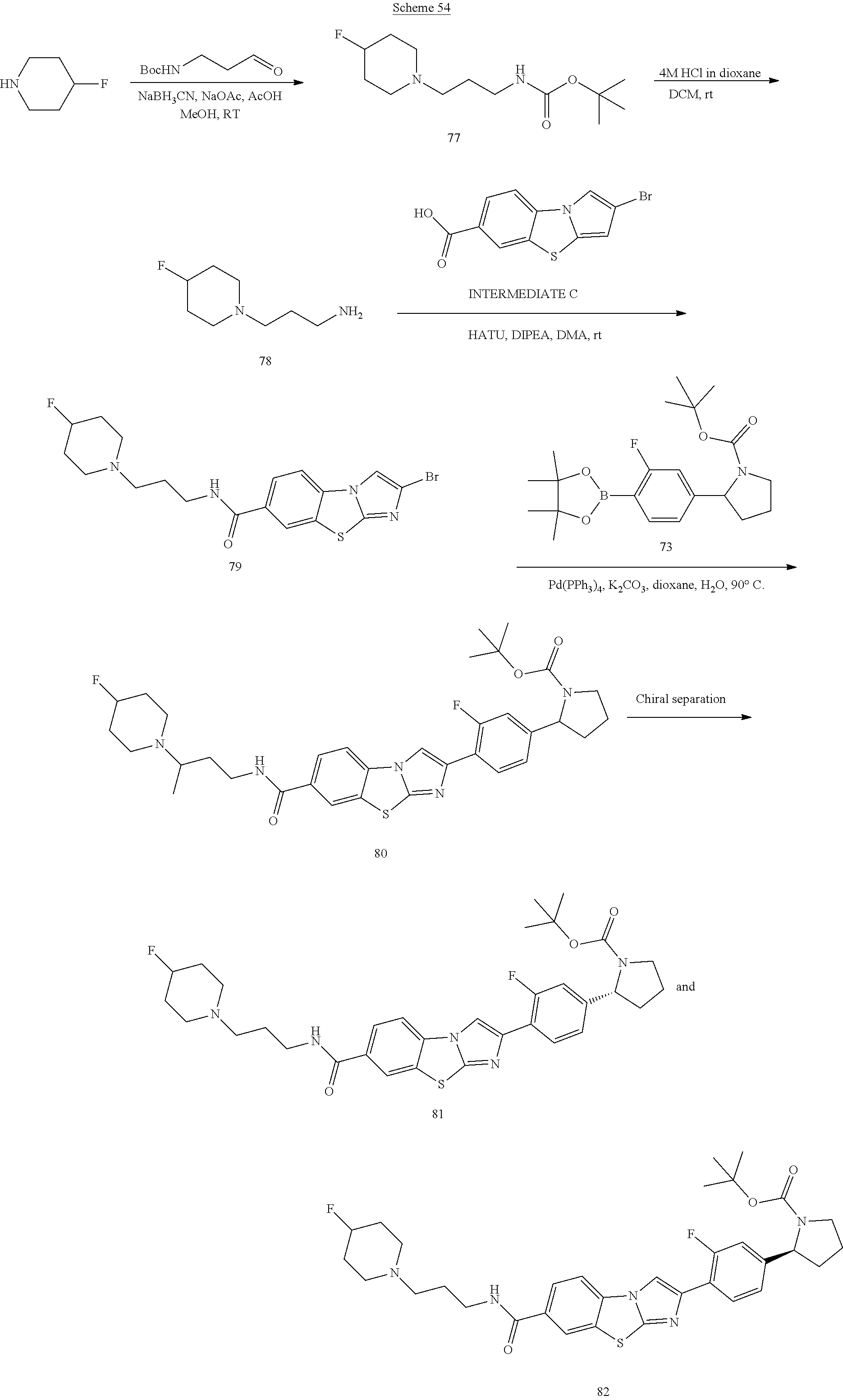

### tert-butyl (3-(4-fluoropiperidin-1-yl)propyl)carbamate

To a stirred mixture of 4-fluoropiperidine hydrochloride (1.00 g, 7.16 mmol) and tert-butyl (3-oxopropyl)carbamate (1.36 g, 7.85 mmol) in methanol (10 mL) were added sodium acetate (1.77 g, 21.58 mmol) and sodium cyanoborohydride (0.90 g, 14.32 mmol) portion-wise at room temperature. The resulting mixture was stirred for 16 h at room temperature under a nitrogen atmosphere. The resulting solution was purified by reverse phase flash chromatography with the following conditions: Column, Spherical C18, 20-40 um, 330; Mobile Phase A: water (plus 10 mM ammonium bicarbonate); Mobile Phase B; acetonitrile; Flow rate: 80 m/min; Gradient:40%-60% B in 20 min; Detector: UV 254/220 nm. The fractions containing desired product were collected and concentrated under reduced pressure to afford tert-butyl (3-(4-fluoropiperidin-1-yl)propyl)carbamate as a yellow liquid.

Yield 0.80 g (43%). ¹H NMR (400 MHz, DMSO) δ 6.79 (t, J=5.6 Hz, 1H), 4.77-4.52 (m, 1H), 2.92 (dt, J=5.6, 7.2 Hz, 2H), 2.52-2.42 (m, 2H), 2.29-2.18 (m, 4H), 1.94-1.75 (m, 2H), 1.74-1.62 (m, 2H), 1.56-1.47 (m, 2H), 1.37 (s, 9H). m/z: [ESI⁺] 261 (M+H)⁺.

### 3-(4-fluoropiperidin-1-yl)propan-1-amine dihydrochloride

Compound 3-(4-fluoropiperidin-1-yl)propan-1-amine dihydrochloride was prepared from tert-butyl (3-(4-fluoropiperidin-1-yl)propyl)carbamate (1.38 g, 5.30 mmol) following a similar procedure to that described for the synthesis of 4-amino-i-(4-bromo-3-fluorophenyl)butan-1-one hydrochloride, and was isolated as a yellow solid.

Yield 0.96 g (78%). ¹H NMR (400 MHz, DMSO) δ 11.28 (br s, 1H, NH⁺), 8.33 (br s, 3H, NH₃+), 5.11-4.86 (m, 1H), 3.51-3.37 (m, 2H), 3.26-3.17 (m, 2H), 3.11-2.97 (m, 2H), 2.96-2.86 (m, 2H), 2.37-2.25 (m, 1H), 2.25-2.00 (m, 5H). ¹⁹F NMR (376 MHz, DMSO) δ −176.04, −186.56, −186.88. m/z: [ESI⁺] 161 (M+H)⁺.

### 2-bromo-N-(3-(4-fluoropiperidin-1-yl)propyl)benzo[d]imidazo[2,1-b]thiazole-7-carboxamide

Compound 2-bromo-N-(3-(4-fluoropiperidin-1-yl)propyl)benzo[d]imidazo[2,1-b]thiazole-7-carboxamide was prepared from 2-bromobenzo[d]imidazo[2,1-b]thiazole-7-carboxylic acid (1.20 g, 4.04 mmol) and 3-(4-fluoropiperidin-1-yl)propan-1-amine dihydrochloride (0.84 g, 3.60 mmol) following a similar procedure to that described for the synthesis of 2-bromo-N-(3-(piperidin-1-yl)propyl)benzo[d]imidazo[2,1-b]thiazole-7-carboxamide, and was isolated as a white solid.

Yield 0.80 g (51%). ¹H NMR (400 MHz, DMSO) δ 8.72 (t, J=5.6 Hz, 1H), 8.58 (s, 1H), 8.53 (d, J=1.6 Hz, 1H), 8.11 (d, J=8.4 Hz, 1H), 8.03 (dd, J=1.6, 8.4 Hz, 1H), 4.99-4.75 (m, 1H), 3.42-3.33 (m, 2H), 3.16-2.85 (m, 6H), 2.06-1.79 (m, 6H). m/z: [ESI⁺] 439, 441 (M+H)⁺.

### tert-butyl 2-(3-fluoro-4-(7-((3-(4-fluoropiperidin-1-yl)propyl)carbamoyl)benzo[d]imidazo[2,1-b]thiazol-2-yl)phenyl)pyrrolidine-1-carboxylate

Compound tert-butyl 2-(3-fluoro-4-(7-((3-(4-fluoropiperidin-1-yl)propyl)carbamoyl)benzo[d]imidazo[2,1-b]thiazol-2-yl)phenyl)pyrrolidine-1-carboxylate was prepared from 2-bromo-N-(3-(4-fluoropiperidin-1-yl)propyl)benzo[d]imidazo[2,1-b]thiazole-7-carboxamide (700 mg, 1.593 mmol) and tert-butyl 2-(3-fluoro-4-(4,4,5,5-tetramethyl-1,3,2-dioxaborolan-2-yl)phenyl)pyrrolidine-1-carboxylate (810 mg, 2.071 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a white solid.

Yield 480 mg (48%). m/z: [ESI⁺] 624 (M+H)⁺.

### tert-butyl (R)-2-(3-fluoro-4-(7-((3-(4-fluoropiperidin-1-yl)propyl)carbamoyl)benzo[d]imidazo[2,1-b]thiazol-2-yl)phenyl)pyrrolidine-1-carboxylate and tert-butyl (S)-2-(3-fluoro-4-(7-((3-(4-fluoropiperidin-1-yl)propyl)carbamoyl)benzo[d]imidazo[2,1-b]thiazol-2-yl)phenyl)pyrrolidine-1-carboxylate

Tert-butyl 2-(3-fluoro-4-(7-((3-(4-fluoropiperidin-1-yl)propyl)carbamoyl)benzo[d]imidazo[2,1-b]thiazol-2-yl)phenyl)pyrrolidine-1-carboxylate (480 mg, 0.770 mmol) was separated by chiral HPLC with the following conditions: (Column: CHIRALPAK IG, 2×25 cm, 5 um; Mobile Phase A: Hexane (plus 0.5% 2 M NH₃-MeOH, v/v), Mobile Phase B: EtOH; Flow rate: 17 mL/min; Gradient: 50% B in 23 min; Detector: UV 254/220 nm; RT1(min): 13.11; RT2(min): 17.85). The fractions containing the faster eluting peak were concentrated under reduced pressure to afford tert-butyl (R)-2-(3-fluoro-4-(7-((3-(4-fluoropiperidin-1-yl)propyl)carbamoyl)benzo[d]imidazo[2,1-b]thiazol-2-yl)phenyl)pyrrolidine-1-carboxylate as a white solid.

Yield 200 mg (42%). ¹H NMR (400 MHz, CD₃OD) δ 8.51-8.44 (m, 1H), 8.36 (d, J=1.6 Hz, 1H), 8.11-7.97 (m, 3H), 7.14 (d, J=8.0 Hz, 1H), 7.10-7.00 (m, 1H), 4.99-4.85 (m, 1H), 4.79-4.58 (m, 1H), 3.72-3.51 (m, 2H), 3.53-3.44 (m, 2H), 2.73-2.63 (m, 2H), 2.59-2.30 (m, 6H), 2.02-1.77 (m, 8H), 1.50 (s, 3H), 1.24 (s, 6H). NH proton not observed. m/z: [ESI⁺] 624 (M+H)⁺.

The fractions containing the slower eluting peak were concentrated under reduced pressure to afford tert-butyl (S)-2-(3-fluoro-4-(7-((3-(4-fluoropiperidin-1-yl)propyl)carbamoyl)benzo[d]imidazo[2,1-b]thiazol-2-yl)phenyl)pyrrolidine-1-carboxylate as a white solid.

Yield 200 mg (42%). ¹H NMR (400 MHz, CD₃OD) δ 8.51-8.44 (m, 1H), 8.36 (d, J=1.6 Hz, 1H), 8.11-7.97 (m, 3H), 7.14 (d, J=8.0 Hz, 1H), 7.10-7.00 (m, 1H), 4.99-4.85 (m, 1H), 4.79-4.58 (m, 1H), 3.72-3.51 (m, 2H), 3.53-3.44 (m, 2H), 2.73-2.63 (m, 2H), 2.59-2.30 (m, 6H), 2.02-1.77 (m, 8H), 1.50 (s, 3H), 1.24 (s, 6H). NH proton not observed. m/z: [ESI⁺] 624 (M+H)⁺.

### tert-butyl (R)-2-(4-(7-((3-(diethylamino)propyl)carbamoyl)benzo[d]imidazo[2,1-b]thiazol-2-yl)phenyl)pyrrolidine-1-carboxylate and tert-butyl (S)-2-(4-(7-((3-(diethylamino)propyl)carbamoyl)benzo[d]imidazo[2,1-b]thiazol-2-yl)phenyl)pyrrolidine-1-carboxylate

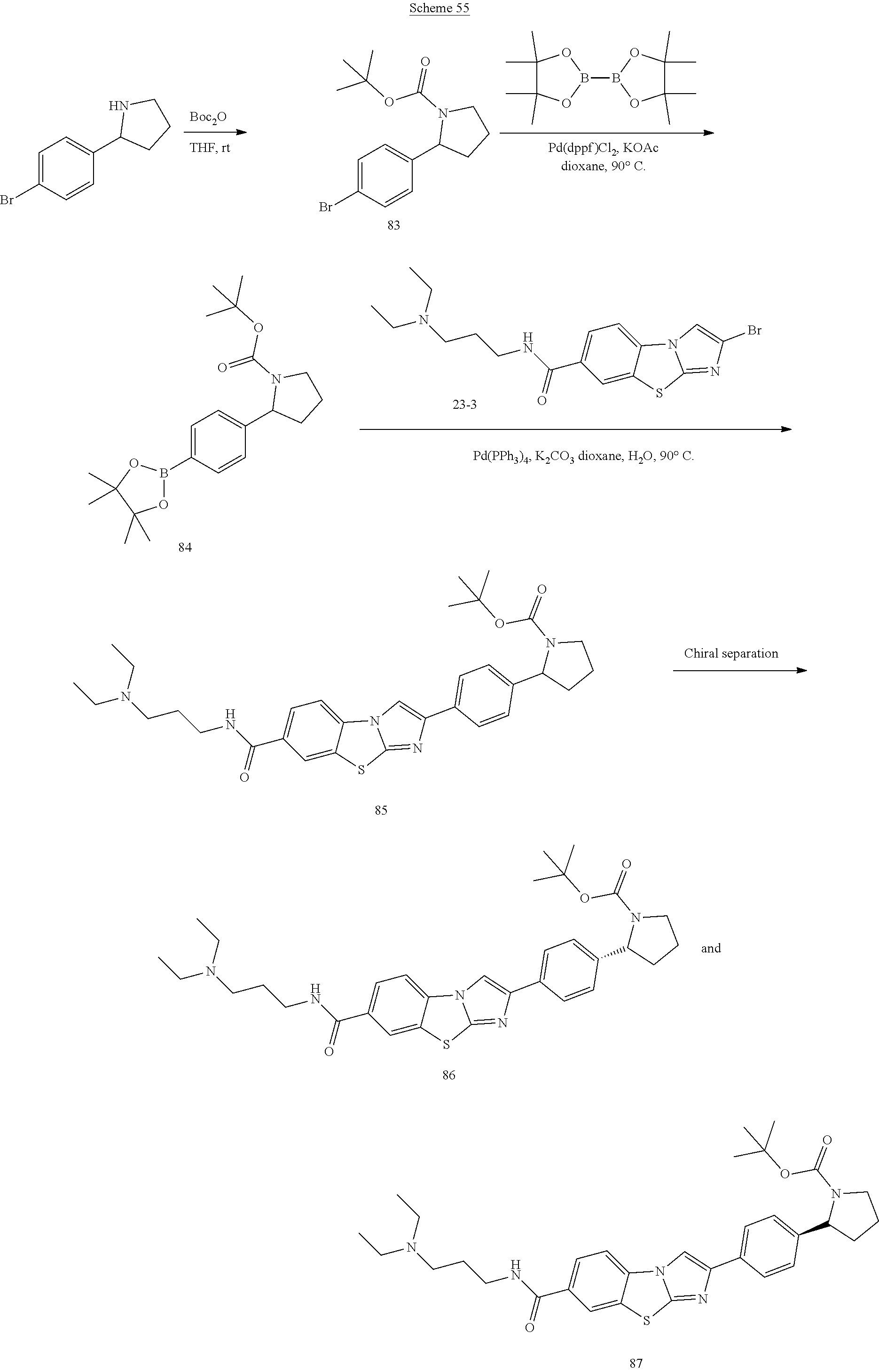

### tert-butyl 2-(4-bromophenyl)pyrrolidine-1-carboxylate

To a stirred solution of 2-(4-bromophenyl)pyrrolidine (8.40 g, 37.15 mmol) in tetrahydrofuran (100 mL) was added di-tert-butyl dicarbonate (9.24 g, 42.34 mmol) at room temperature. The resulting solution was stirred for 2 h at room temperature. The resulting solution was concentrated under reduced pressure. The residue was purified by silica gel column chromatography, eluting with 1%-50% ethyl acetate in petroleum ether to afford tert-butyl 2-(4-bromophenyl)pyrrolidine-1-carboxylate as a white solid.

Yield 12.00 g (99%). ¹H NMR (400 MHz, CDCl₃) δ 7.43 (d, J=8.4 Hz, 2H), 7.07 (d, J=8.4 Hz, 2H), 4.97-4.83 (m, 0.3H), 4.83-4.66 (m, 0.7H), 3.72-3.27 (m, 2H), 2.42-2.22 (m, 1H), 1.97-1.84 (m, 2H), 1.83-1.71 (m, 1H), 1.47 (s, 3H), 1.22 (s, 6H). m/z: [ESI⁺] 270, 272 (M+H−56)⁺.

### tert-butyl 2-(4-(4,4,5,5-tetramethyl-1,3,2-dioxaborolan-2-yl)phenyl)pyrrolidine-1-carboxylate

Compound tert-butyl 2-(4-(4,4,5,5-tetramethyl-1,3,2-dioxaborolan-2-yl)phenyl)pyrrolidine-1-carboxylate was prepared from tert-butyl 2-(4-bromophenyl)pyrrolidine-1-carboxylate (10.00 g, 30.65 mmol,) following a similar procedure to that described for the synthesis of tert-butyl (3-cyclopropyl-4-(4,4,5,5-tetramethyl-1,3,2-dioxaborolan-2-yl)benzyl)carbamate, and was isolated as a white solid

Yield 10.75 g (94%). ¹H NMR (400 MHz, CDCl₃) δ 7.76 (d, J=7.6 Hz, 2H), 7.19 (d, J=7.6 Hz, 2H), 5.02-4.89 (m, 0.3H), 4.89-4.75 (m, 0.7H), 3.71-3.57 (m, 2H), 2.40-2.24 (m, 1H), 1.96-1.74 (m, 3H), 1.46 (s, 3H), 1.36 (s, 12H), 1.21 (s, 6H). m/z: [ESI⁺] 374 (M+H)⁺.

### tert-butyl 2-(4-(7-((3-(diethylamino)propyl)carbamoyl)benzo[d]imidazo[2,1-b]thiazol-2-yl)phenyl)pyrrolidine-1-carboxylate

Compound tert-butyl 2-(4-(7-((3-(diethylamino)propyl)carbamoyl)benzo[d]imidazo[2,1-b]thiazol-2-yl)phenyl)pyrrolidine-1-carboxylate was prepared from 2-bromo-N-(3-(diethylamino)propyl)benzo[d]imidazo[2,1-b]thiazole-7-carboxamide (2.10 g, 5.13 mmol,) and tert-butyl 2-(4-(4,4,5,5-tetramethyl-1,3,2-dioxaborolan-2-yl)phenyl)pyrrolidine-1-carboxylate (3.83 g, 10.26 mmol,) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a light yellow solid.

Yield 0.25 g (8%). 1H NMR (400 MHz, DMSO) δ 8.79 (s, 1H), 8.68 (br s, 1H), 8.49 (d, J=1.6 Hz, 1H), 8.24 (d, J=8.0 Hz, 1H), 8.04 (dd, J=1.6, 8.0 Hz, 1H), 7.82 (d, J=8.0 Hz, 2H), 7.25 (d, J=8.0 Hz, 2H), 4.91-4.69 (m, 1H), 3.63-3.43 (m, 2H), 3.38-3.31 (m, 2H), 2.72-2.63 (m, 6H), 1.95-1.64 (m, 6H), 1.41 (s, 3H), 1.12 (s, 6H), 1.17-0.92 (m, 6H). m/z: [ESI⁺] 576 (M+H)⁺.

### tert-butyl (R)-2-(4-(7-((3-(diethylamino)propyl)carbamoyl)benzo[d]imidazo[2,1-b]thiazol-2-yl)phenyl)pyrrolidine-1-carboxylate and tert-butyl (S)-2-(4-(7-((3-(diethylamino)propyl)carbamoyl)benzo[d]imidazo[2,1-b]thiazol-2-yl)phenyl)pyrrolidine-1-carboxylate

Tert-butyl 2-(4-(7-((3-(diethylamino)propyl)carbamoyl)benzo[d]imidazo[2,1-b]thiazol-2-yl)phenyl)pyrrolidine-1-carboxylate (428 mg, 0.743 mmol) was separated by chiral HPLC with the following conditions: (Column: CHIRALPAK IG, 2×25 cm, 5 um; Mobile Phase A: Hexane (0.5% 2 M NH₃-MeOH), Mobile Phase B: EtOH; Flow rate: 20 mL/min; Gradient: 40% B to 40% B in 23 min; Wave Length: 220/254 nm; RT1(min): 13.63; RT2(min): 17.91). The fractions containing the faster eluting peak were concentrated under reduced pressure to afford tert-butyl (R)-2-(4-(7-((3-(diethylamino)propyl)carbamoyl)benzo[d]imidazo[2,1-b]thiazol-2-yl)phenyl)pyrrolidine-1-carboxylate as a white solid.

Yield 130 mg (30%). ¹H NMR (400 MHz, DMSO) δ 8.79 (s, 1H), 8.68 (br s, 1H), 8.49 (d, J=1.6 Hz, 1H), 8.24 (d, J=8.0 Hz, 1H), 8.04 (dd, J=1.6, 8.0 Hz, 1H), 7.82 (d, J=8.0 Hz, 2H), 7.25 (d, J=8.0 Hz, 2H), 4.91-4.69 (m, 1H), 3.63-3.43 (m, 2H), 3.38-3.31 (m, 2H), 2.72-2.63 (m, 6H), 1.95-1.64 (m, 6H), 1.41 (s, 3H), 1.12 (s, 6H), 1.17-0.92 (m, 6H). m/z: [ESI⁺] 576 (M+H)⁺.

The fractions containing the slower eluting peak were concentrated under reduced pressure to afford tert-butyl (S)-2-(4-(7-((3-(diethylamino)propyl)carbamoyl)benzo[d]imidazo[2,1-b]thiazol-2-yl)phenyl)pyrrolidine-1-carboxylate as a white solid.

Yield 148 mg (35%). ¹H NMR (400 MHz, DMSO) δ 8.79 (s, 1H), 8.68 (br s, 1H), 8.49 (d, J=1.6 Hz, 1H), 8.24 (d, J=8.0 Hz, 1H), 8.04 (dd, J=1.6, 8.0 Hz, 1H), 7.82 (d, J=8.0 Hz, 2H), 7.25 (d, J=8.0 Hz, 2H), 4.91-4.69 (m, 1H), 3.63-3.43 (m, 2H), 3.38-3.31 (m, 2H), 2.72-2.63 (m, 6H), 1.95-1.64 (m, 6H), 1.41 (s, 3H), 1.12 (S, 6H), 1.17-0.92 (m, 6H). m/z: [ESI⁺] 576 (M+H)⁺.

### tert-butyl 2-(4-(7-((3-(piperidin-1-yl)propyl)carbamoyl)benzo[d]imidazo[2,1-b]thiazol-2-yl)phenyl)pyrrolidine-1-carboxylate

### tert-butyl (R)-2-(4-(7-((3-(piperidin-1-yl)propyl)carbamoyl)benzo[d]imidazo[2,1-b]thiazol-2-yl)phenyl)pyrrolidine-1-carboxylate

### tert-butyl (S)-2-(4-(7-((3-(piperidin-1-yl)propyl)carbamoyl)benzo[d]imidazo[2,1-b]thiazol-2-yl)phenyl)pyrrolidine-1-carboxylate

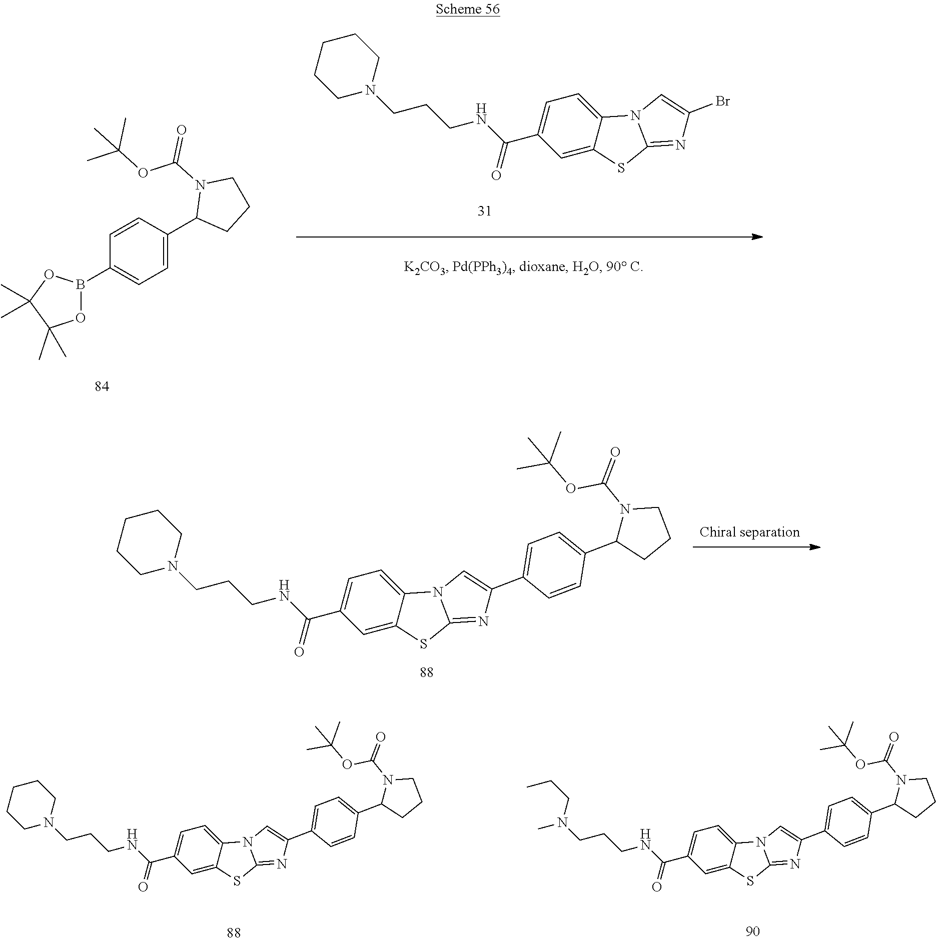 [SMILES: CC(C)(C)OC(=O)N1CCCC1c1ccc(B2OC(C)(C)C(C)(C)O2)cc1 ; O=C(NCCCN1CCCCC1)c1ccc2c(c1)sc1nc(Br)cn12 ; CC(C)(C)OC(=O)N1CCCC1c1ccc(-c2cn3c(n2)sc2cc(C(=O)NCCCN4CCCCC4)ccc23)cc1 ; CC(C)(C)OC(=O)N1CCCC1c1ccc(-c2cn3c(n2)sc2cc(C(=O)NCCCN4CCCCC4)ccc23)cc1 ; CCCN(C)CCCNC(=O)c1ccc2c(c1)sc1nc(-c3ccc(C4CCCN4C(=O)OC(C)(C)C)cc3)cn12]

### tert-butyl 2-(4-(7-((3-(piperidin-1-yl)propyl)carbamoyl)benzo[d]imidazo[2,1-b]thiazol-2-yl)phenyl)pyrrolidine-1-carboxylate

Compound tert-butyl 2-(4-(7-((3-(piperidin-1-yl)propyl)carbamoyl)benzo[d]imidazo[2,1-b]thiazol-2-yl)phenyl)pyrrolidine-1-carboxylate was prepared from tert-butyl 2-(4-(4,4,5,5-tetramethyl-1,3,2-dioxaborolan-2-yl)phenyl)pyrrolidine-1-carboxylate (532 mg, 1.425 mmol) and 2-bromo-N-(3-(piperidin-1-yl)propyl)benzo[d]imidazo[2,1-b]thiazole-7-carboxamide (400 mg, 0.949 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a white solid.

Yield 400 mg (72%). ¹H NMR (400 MHz, CDCl₃) δ 8.56 (br s, 1H), 8.47 (s, 1H), 8.18 (d, J=8.4 Hz, 1H), 7.99 (s, 1H), 7.82 (d, J=8.0 Hz, 2H), 7.67 (d, J=8.4 Hz, 1H), 7.24 (d, J=8.0 Hz, 2H), 5.11-4.59 (m, 1H), 3.72-3.55 (m, 6H), 3.18-3.06 (m, 2H), 2.43-2.28 (m, 1H), 2.24-2.15 (m, 2H), 2.00-1.85 (m, 9H), 1.78-1.58 (m, 2H), 1.49 (s, 3H), 1.22 (s, 6H). m/z: [ESI⁺] 588 (M+H)⁺.

### tert-butyl (R)-2-(4-(7-((3-(piperidin-1-yl)propyl)carbamoyl)benzo[d]imidazo[2,1-b]thiazol-2-yl)phenyl)pyrrolidine-1-carboxylate and tert-butyl (S)-2-(4-(7-((3-(piperidin-1-yl)propyl)carbamoyl)benzo[d]imidazo[2,1-b]thiazol-2-yl)phenyl)pyrrolidine-1-carboxylate

Tert-butyl 2-(4-(7-((3-(piperidin-1-yl)propyl)carbamoyl)benzo[d]imidazo[2,1-b]thiazol-2-yl)phenyl)pyrrolidine-1-carboxylate (1.45 g, 2.47 mmol) was separated by SFC with the following conditions (Column: (R, R)-WHELK-01-Kromasil, 5×25 cm, 5 μm; Mobile Phase A: CO₂, Mobile Phase B: methanol:acetonitrile:dichloromethane=1:1:1 (0.1% 2 M NH₃-MeOH); Flow rate: 250 m/min; Gradient: isocratic 50% B; Column Temperature (° C.): 35; Back Pressure (bar): 100; wave Length: 230 nm; RT1(min): 12.11; RT2(min): 24.88). The fractions containing the faster eluting peak were concentrated under reduced pressure to afford tert-butyl (R)-2-(4-(7-((3-(piperidin-1-yl)propyl)carbamoyl)benzo[d]imidazo[2,1-b]thiazol-2-yl)phenyl)pyrrolidine-1-carboxylate as a yellow solid.

Yield 0.49 g (34%). ¹H NMR (400 MHz, CDCl₃) δ 8.56 (br s, 1H), 8.47 (s, 1H), 8.18 (d, J=8.4 Hz, 1H), 7.99 (s, 1H), 7.82 (d, J=8.0 Hz, 2H), 7.67 (d, J=8.4 Hz, 1H), 7.24 (d, J=8.0 Hz, 2H), 5.11-4.59 (m, 1H), 3.72-3.55 (m, 6H), 3.18-3.06 (m, 2H), 2.43-2.28 (m, 1H), 2.24-2.15 (m, 2H), 2.00-1.85 (m, 9H), 1.78-1.58 (m, 2H), 1.49 (s, 3H), 1.22 (s, 6H). m/z: [ESI⁺] 588 (M+H)⁺.

The fractions containing the slower eluting peak were concentrated under reduced pressure to afford tert-butyl (S)-2-(4-(7-((3-(piperidin-1-yl)propyl)carbamoyl)benzo[d]imidazo[2,1-b]thiazol-2-yl)phenyl)pyrrolidine-1-carboxylate as a yellow solid.

Yield 0.46 g (32%). ¹H NMR (400 MHz, CDCl₃) δ 8.56 (br s, 1H), 8.47 (s, 1H), 8.18 (d, J=8.4 Hz, 1H), 7.99 (s, 1H), 7.82 (d, J=8.0 Hz, 2H), 7.67 (d, J=8.4 Hz, 1H), 7.24 (d, J=8.0 Hz, 2H), 5.11-4.59 (m, 1H), 3.72-3.55 (m, 6H), 3.18-3.06 (m, 2H), 2.43-2.28 (m, 1H), 2.24-2.15 (m, 2H), 2.00-1.85 (m, 9H), 1.78-1.58 (m, 2H), 1.49 (s, 3H), 1.22 (s, 6H). m/z: [ESI⁺] 588 (M+H)⁺.

### tert-butyl cyclopropyl(4-(7-((3-(diethylamino)propyl)carbamoyl)benzo[d]imidazo[2,1-b]thiazol-2-yl)-3-fluorobenzyl)carbamate

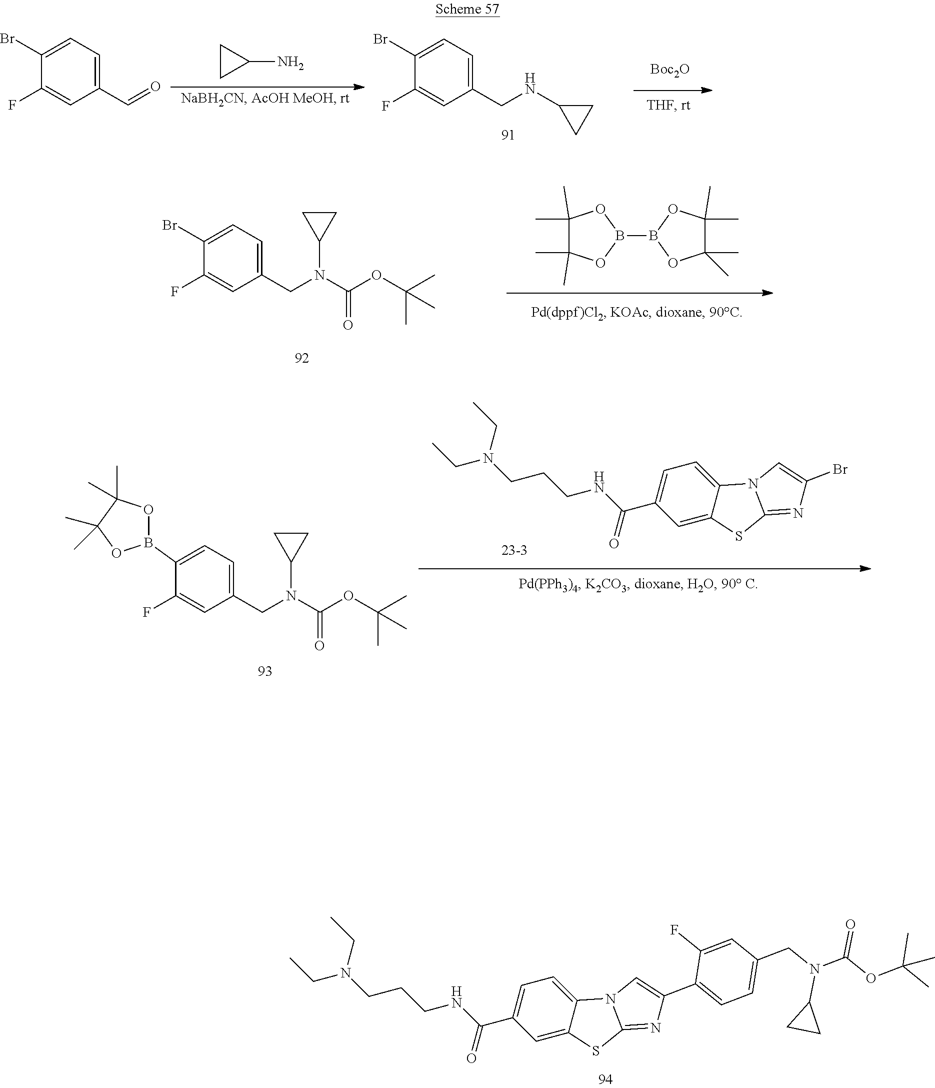

### N-(4-bromo-3-fluorobenzyl)cyclopropanamine

Compound N-(4-bromo-3-fluorobenzyl)cyclopropanamine was prepared from 4-bromo-3-fluorobenzaldehyde (20.00 g, 98.52 mmol) and cyclopropanamine (16.87 g, 295.45 mmol) following a similar procedure to that described for the synthesis of tert-butyl (3-(4-fluoropiperidin-1-yl)propyl)carbamate, and was isolated as a colorless liquid.

Yield 19.00 g (79%). ¹H NMR (400 MHz, CDCl₃) δ 7.55-7.47 (m, 1H), 7.14 (dd, J=2.0, 9.6 Hz, 1H), 7.01 (dd, J=2.0, 8.0 Hz, 1H), 3.83 (s, 2H), 2.21-2.12 (m, 1H), 0.50-0.43 (m, 2H), 0.42-0.36 (m, 2H). NH proton not observed. m/z: [ESI⁺] 244, 246 (M+H)⁺.

### tert-butyl (4-bromo-3-fluorobenzyl)(cyclopropyl)carbamate

Compound tert-butyl (4-bromo-3-fluorobenzyl)(cyclopropyl)carbamate was prepared from N-(4-bromo-3-fluorobenzyl)cyclopropanamine (19.00 g, 77.84 mmol) following a similar procedure to that described for the synthesis of tert-butyl 2-(4-bromophenyl)pyrrolidine-1-carboxylate, and was isolated as a colorless liquid.

Yield 25.00 g (93%). ¹H NMR (400 MHz, CDCl₃) δ 7.55-7.45 (m, 1H), 7.03 (dd, J=2.0, 9.6 Hz, 1H), 6.93 (dd, J=2.0, 8.0 Hz, 1H), 4.39 (s, 2H), 2.51-2.47 (m, 1H), 1.48 (s, 9H), 0.79-0.71 (m, 2H), 0.68-0.60 (m, 2H). m/z: [ESI⁺] 288, 290 (M+H−56)⁺.

### tert-butyl cyclopropyl(3-fluoro-4-(4,4,5,5-tetramethyl-1,3,2-dioxaborolan-2-yl)benzyl)carbamate

Compound tert-butyl cyclopropyl(3-fluoro-4-(4,4,5,5-tetramethyl-1,3,2-dioxaborolan-2-yl)benzyl)carbamate was prepared from tert-butyl (4-bromo-3-fluorobenzyl)(cyclopropyl)carbamate (4.00 g, 11.62 mmol) following a similar procedure to that described for the synthesis of tert-butyl (3-cyclopropyl-4-(4,4,5,5-tetramethyl-1,3,2-dioxaborolan-2-yl)benzyl)carbamate, and was isolated as a light green oil.

Yield 4.10 g (90%). ¹H NMR (400 MHz, CDCl₃) δ 7.75-7.66 (m, 1H), 7.03 (d, J=7.6 Hz, 1H), 6.92 (d, J=10.0 Hz, 1H), 4.44 (s, 2H), 2.53-2.49 (m, 1H), 1.47 (s, 9H), 1.38 (s, 12H), 0.77-0.70 (M, 2H), 0.67-0.61 (m, 2H). m/z: [ESI⁺] 336 (M+H−56)⁺.

### tert-butyl cyclopropyl(4-(7-((3-(diethylamino)propyl)carbamoyl)benzo[d]imidazo[2,1-b]thiazol-2-yl)-3-fluorobenzyl)carbamate

Compound tert-butylcyclopropyl(4-(7-((3-(diethylamino)propyl)carbamoyl)benzo[d]imidazo[2,1-b]thiazol-2-yl)-3-fluorobenzyl)carbamate was prepared from 2-bromo-N-(3-(diethylamino)propyl)benzo[d]imidazo[2,1-b]thiazole-7-carboxamide (2.78 g, 6.79 mmol) and tert-butyl cyclopropyl(3-fluoro-4-(4,4,5,5-tetramethyl-1,3,2-dioxaborolan-2-yl)benzyl)carbamate (4.00 g, 10.22 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a dark yellow oil.

Yield 2.40 g (60%). ¹H NMR (400 MHz, CDCl₃) δ 8.68 (t, J=5.6 Hz, 1H), 8.43 (d, J=1.6 Hz, 1H), 8.39 (s, 1H), 8.16-8.10 (m, 2H), 7.62 (d, J=8.4 Hz, 1H), 7.09 (dd, J=1.6, 8.0 Hz, 1H), 7.01 (dd, J=1.6, 12.0 Hz, 1H), 4.42 (s, 2H), 3.67-3.55 (m, 2H), 3.24-3.13 (m, 6H), 2.53-2.49 (m, 1H), 2.27-2.13 (m, 2H), 1.46 (s, 9H), 1.39-1.32 (t, J=7.2 Hz, 6H), 0.79-0.70 (m, 2H), 0.68-0.59 (m, 2H). m/z: [ESI⁺] 594 (M+H)⁺.

### tert-butyl cyclopropyl(4-(7-((3-(diethylamino)propyl)carbamoyl)benzo[d]imidazo[2,1-b]thiazol-2-yl)-2,5-difluorobenzyl)carbamate

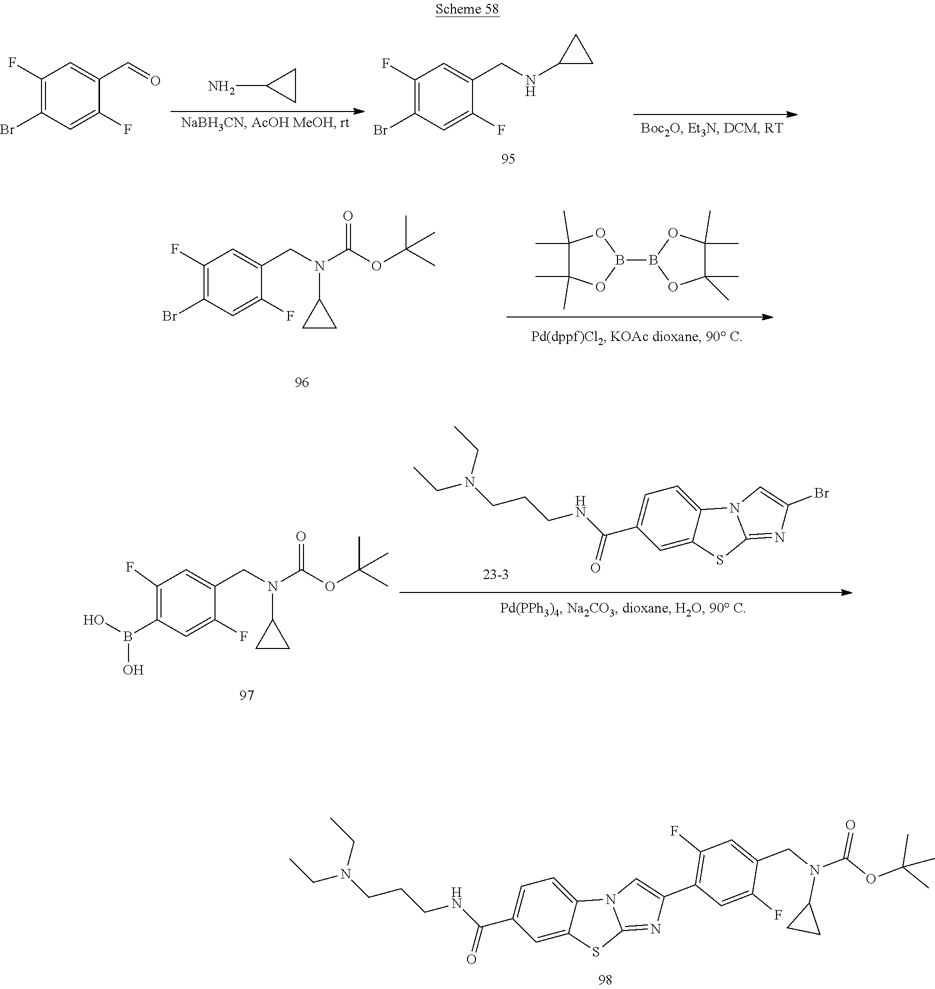

### N-(4-bromo-2,5-difluorobenzyl)cyclopropanamine

Compound N-(4-bromo-2,5-difluorobenzyl)cyclopropanamine was prepared from 4-bromo-2,5-difluorobenzaldehyde (5.00 g, 22.62 mmol) and cyclopropanamine (2.58 g, 45.18 mmol) following a similar procedure to that described for the synthesis of tert-butyl (3-(4-fluoropiperidin-1-yl)propyl)carbamate, and was isolated as a light yellow oil.

Yield 5.50 g (93%). ¹H NMR (400 MHz, CDCl₃) δ 7.27 (dd, J=5.6, 8.8 Hz, 1H), 7.17 (dd, J=6.0, 8.8 Hz, 1H), 3.86 (s, 2H), 2.18-2.09 (m, 1H), 0.51-0.44 (m, 2H), 0.43-0.37 (m, 2H). NH proton not observed. m/z: [ESI⁺] 262, 264 (M+H)⁺.

### tert-butyl (4-bromo-2,5-difluorobenzyl)(cyclopropyl)carbamate

Compound tert-butyl (4-bromo-2,5-difluorobenzyl)(cyclopropyl)carbamate was prepared from N-(4-bromo-2,5-difluorobenzyl)cyclopropanamine (1.00 g, 3.82 mmol) following a similar procedure to that described for the synthesis of tert-butyl 2-(4-bromophenyl)pyrrolidine-1-carboxylate, and was isolated as a colorless oil.

Yield 1.00 g (72%). ¹H NMR (400 MHz, CDCl₃) δ 7.27 (dd, J=5.6, 8.8 Hz, 1H), 7.02 (dd, J=6.0, 8.8 Hz, 1H), 4.44 (s, 2H), 2.53-2.49 (m, 1H), 1.48 (s, 9H), 0.76-0.70 (m, 2H), 0.69-0.57 (m, 2H). m/z: [ESI⁺] 306, 308 (M+H−56)⁺.

### (4-(((tert-butoxycarbonyl)(cyclopropyl)amino)methyl)-2,5-difluorophenyl)boronic acid

Compound (4-(((tert-butoxycarbonyl)(cyclopropyl)amino)methyl)-2,5-difluorophenyl)boronic acid was prepared from tert-butyl (4-bromo-2,5-difluorobenzyl)(cyclopropyl)carbamate (1.00 g, 2.76 mmol) following a similar procedure to that described for the synthesis of tert-butyl (3-cyclopropyl-4-(4,4,5,5-tetramethyl-1,3,2-dioxaborolan-2-yl)benzyl)carbamate, and was isolated as a white solid.

Yield 0.25 g (28%). ¹H NMR (400 MHz, DMSO) δ 7.29 (dd, J=4.8, 10.0 Hz, 1H), 6.87 (dd, J=9.2, 5.6 Hz, 1H), 4.39 (s, 2H), 2.47-2.43 (m, 1H), 1.39 (s, 9H), 0.71-0.64 (m, 2H), 0.64-0.57 (m, 2H). Boronic Acid protons not observed. m/z: [ESI⁺] 272 (M+1-56)⁺.

### tert-butyl cyclopropyl(4-(7-((3-(diethylamino)propyl)carbamoyl)benzo[d]imidazo[2,1-b]thiazol-2-yl)-2,5-difluorobenzyl)carbamate

Compound tert-butyl cyclopropyl(4-(7-((3-(diethylamino)propyl)carbamoyl)benzo[d]imidazo[2,1-b]thiazol-2-yl)-2,5-difluorobenzyl)carbamate was prepared from (4-(((tert-butoxycarbonyl)(cyclopropyl)amino)methyl)-2,5-difluorophenyl)boronic acid (0.48 g, 1.47 mmol) and 2-bromo-N-(3-(diethylamino)propyl)benzo[d]imidazo[2,1-b]thiazole-7-carboxamide (0.30 g, 0.73 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as an off-white solid.

Yield 0.20 g (45%). ¹H NMR (400 MHz, CD₃OD) δ 8.54 (d, J=3.6 Hz, 1H), 8.37 (d, J=1.6 Hz, 1H), 8.06 (d, J=8.4 Hz, 1H), 8.02 (dd, J=1.6, 8.4 Hz, 1H), 7.82 (dd, J=6.0, 10.8 Hz, 1H), 7.11 (dd, J=4.4, 9.2 Hz, 1H), 4.50 (s, 2H), 3.52-3.42 (m, 2H), 2.77-2.61 (m, 6H), 2.61-2.46 (m, 1H), 1.93-1.80 (m, 2H), 1.50 (s, 9H), 1.12 (t, J=7.2 Hz, 6H), 0.83-0.74 (m, 2H), 0.74-0.61 (m, 2H). NH proton not observed. m/z: [ESI⁺] 612 (M+H)⁺.

### tert-butyl (1-(4-(7-((3-(diethylamino)propyl)carbamoyl)benzo[d]imidazo[2,1-b]thiazol-2-yl)phenyl)cyclopropyl)carbamate

Compound tert-butyl (1-(4-(7-((3-(diethylamino)propyl)carbamoyl)benzo[d]imidazo[2,1-b]thiazol-2-yl)phenyl)cyclopropyl)carbamate was prepared from 2-bromo-N-(3-(diethylamino)propyl)benzo[d]imidazo[2,1-b]thiazole-7-carboxamide (0.50 g, 1.22 mmol) and (4-(1-((tert-butoxycarbonyl)amino)cyclopropyl)phenyl)boronic acid (0.51 g, 1.84 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as an off-white solid.

Yield 0.20 g (29%). ¹H NMR (400 MHz, CDCl₃) δ 9.09 (br s, 1H), 8.27 (s, 1H), 8.03-7.93 (m, 2H), 7.82 (d, J=8.4 Hz, 2H), 7.65 (d, J=8.4 Hz, 1H), 7.30 (d, J=8.4 Hz, 2H), 5.33 (br s, 1H), 3.69-3.60 (m, 2H), 2.81-2.65 (m, 6H), 1.98-1.83 (m, 2H), 1.48 (s, 9H), 1.34-1.22 (m, 4h), 1.14 (t, J=7.2 Hz, 6H). m/z: [ESI⁺] 562 (M+H)⁺.

### tert-butyl (3-(7-((3-(diethylamino)propyl)carbamoyl)benzo[d]imidazo[2,1-b]thiazol-2-yl)benzyl)carbamate

Compound tert-butyl (3-(7-((3-(diethylamino)propyl)carbamoyl)benzo[d]imidazo[2,1-b]thiazol-2-yl)benzyl)carbamate was prepared from 2-bromo-N-(3-(diethylamino)propyl)benzo[d]imidazo[2,1-b]thiazole-7-carboxamide (400 mg, 0.977 mmol) and (3-(((tert-butoxycarbonyl)amino)methyl) phenyl)boronic acid (368 mg, 1.466 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a light yellow solid.

Yield 400 mg (76%). ¹H NMR (400 MHz, DMSO) δ 8.80 (s, 1H), 8.66 (t, J=5.6 Hz, 1H), 8.49 (d, J=1.6 Hz, 1H), 8.08 (d, J=8.4 Hz, 1H), 8.02 (dd, J=1.6, 8.4 Hz, 1H), 7.79 (s, 1H), 7.73 (d, J=7.6 Hz, 1H), 7.39 (dd, J=7.6, 7.6 Hz, 1H), 7.18 (d, J=7.6 Hz, 1H), 7.07 (t, J=5.2 Hz, 1H), 4.19 (d, J=5.2 Hz, 2H), 3.40-3.28 (m, 2H), 2.52-2.37 (m, 6H), 1.74-1.60 (m, 2H), 1.41 (s, 9h), 0.95 (t, J=7.1 Hz, 6H). m/z: [ESI⁺] 536 (M+H)⁺.

### tert-butyl (1-(4-(7-((3-(piperidin-1-yl)propyl)carbamoyl)benzo[d]imidazo[2,1-b]thiazol-2-yl)phenyl)cyclopropyl)carbamate

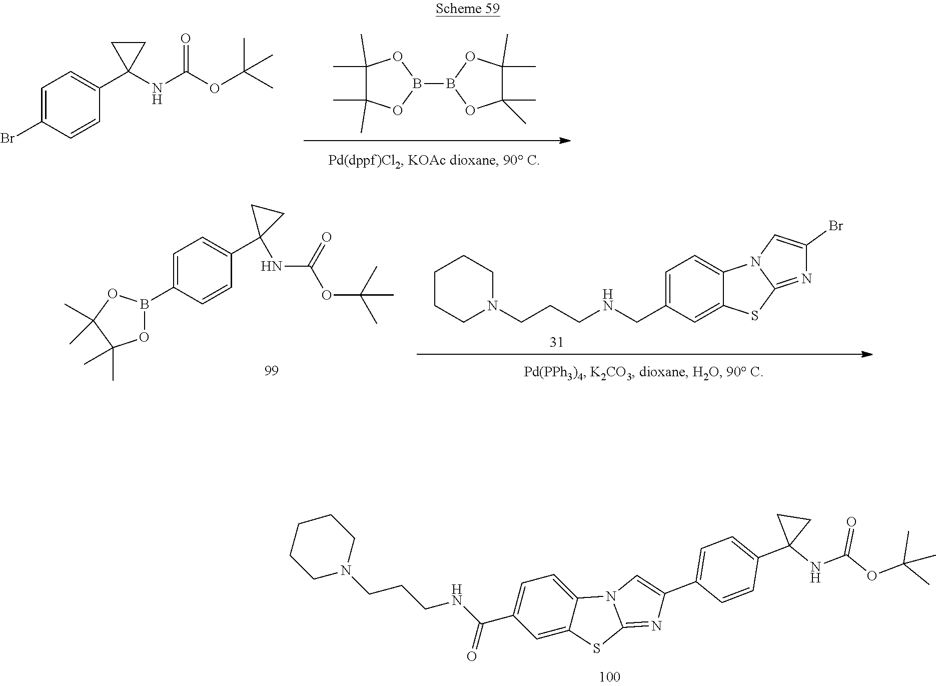

### tert-butyl (1-(4-(4,4,5,5-tetramethyl-1,3,2-dioxaborolan-2-yl)phenyl)cyclopropyl)carbamate

Compound tert-butyl (1-(4-(4,4,5,5-tetramethyl-1,3,2-dioxaborolan-2-yl)phenyl)cyclopropyl)carbamate was prepared from tert-butyl (1-(4-bromophenyl)cyclopropyl)carbamate (6.00 g, 19.22 mmol) following a similar procedure to that described for the synthesis of tert-butyl (3-cyclopropyl-4-(4,4,5,5-tetramethyl-1,3,2-dioxaborolan-2-yl)benzyl)carbamate, and was isolated as an off-white solid.

Yield 6.20 g (90%). ¹H NMR (400 MHz, CDCl₃) δ 7.76 (d, J=8.0 Hz, 2H), 7.22 (d, J=8.0 Hz, 2H), 5.29 (br s, 1H), 1.46 (s, 9H), 1.35 (s, 12H), 1.33-1.24 (m, 4H). m/z: [ESI⁺] 304 (M+H−56)⁺.

### tert-butyl (1-(4-(7-((3-(piperidin-1-yl)propyl)carbamoyl)benzo[d]imidazo[2,1-b]thiazol-2-yl)phenyl)cyclopropyl)carbamate

Compound tert-butyl (1-(4-(7-((3-(piperidin-1-yl)propyl)carbamoyl)benzo[d]imidazo[2,1-b]thiazol-2-yl)phenyl)cyclopropyl)carbamate was prepared from 2-bromo-N-(3-(piperidin-1-yl)propyl)benzo[d]imidazo[2,1-b]thiazole-7-carboxamide (1.00 g, 2.37 mmol) and tert-butyl (1-(4-(4,4,5,5-tetramethyl-1,3,2-dioxaborolan-2-yl)phenyl)cyclopropyl)carbamate (1.20 g, 3.34 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a light yellow solid.

Yield 0.99 g (73%). ¹H NMR (400 MHz, DMSO) δ 8.76 (s, 1H), 8.65 (t, J=5.6 Hz, 1H), 8.48 (d, J=1.6 Hz, 1H), 8.05 (d, J=8.4 Hz, 1H), 8.02 (dd, J=1.6, 8.4 Hz, 1H), 7.78 (d, J=8.0 Hz, 2H), 7.70 (br s, 1H), 7.20 (d, J=8.0 Hz, 2H), 3.38-3.26 (m, 2H), 2.52-2.43 (m, 6H), 1.81-1.68 (m, 2H), 1.61-1.49 (m, 4H), 1.41 (s, 9H), 1.33-1.23 (m, 2H), 1.21-1.11 (m, 4H). m/z: [ESI⁺] 574 (M+H)⁺.

### tert-butyl (4-(7-((3-(piperidin-1-yl)propyl)carbamoyl)benzo[d]imidazo[2,1-b]thiazol-2-yl)benzyl)carbamate

Compound tert-butyl (4-(7-((3-(piperidin-1-yl)propyl)carbamoyl)benzo[d]imidazo[2,1-b]thiazol-2-yl)benzyl)carbamate was prepared from 2-bromo-N-(3-(piperidin-1-yl)propyl)benzo[d]imidazo[2,1-b]thiazole-7-carboxamide (400 mg, 0.949 mmol) and (4-(((tert-butoxycarbonyl)amino)methyl)phenyl)boronic acid (286 mg, 1.139 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as an off-white solid.

Yield 200 mg (38%). ¹H NMR (400 MHz, CDCl₃) δ 8.66 (t, J=5.6 Hz, 1H), 8.57 (s, 1H), 8.49 (d, J=1.6 Hz, 1H), 8.21 (dd, J=1.6, 8.4 Hz, 1H), 7.85 (d, J=8.0 Hz, 2H), 7.67 (d, J=8.4 Hz, 1H), 7.36 (d, J=8.0 Hz, 2H), 4.92 (br s, 1H), 4.42-4.31 (m, 2H), 3.69-3.57 (m, 2H), 3.14-3.07 (m, 6H), 2.27-2.09 (m, 2H), 2.01-1.91 (m, 4H), 1.81-1.59 (m, 2H), 1.50 (s, 9H). m/z: [ESI⁺] 548 (M+H)⁺.

### tert-butyl (2-fluoro-4-(7-((3-(piperidin-1-yl)propyl)carbamoyl)benzo[d]imidazo[2,1-b]thiazol-2-yl)benzyl)carbamate

Compound tert-butyl (2-fluoro-4-(7-((3-(piperidin-1-yl)propyl)carbamoyl)benzo[d]imidazo[2,1-b]thiazol-2-yl)benzyl)carbamate was prepared from 2-bromo-N-(3-(piperidin-1-yl)propyl)benzo[d]imidazo[2,1-b]thiazole-7-carboxamide (300 mg, 0.712 mmol) and (4-(((tert-butoxycarbonyl)amino)methyl)-3-fluorophenyl)boronic acid (383 mg, 1.423 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as an off-white solid.

Yield 150 mg (37%). ¹H NMR (400 MHz, DMSO) δ 8.87 (s, 1H), 8.68 (t, J=5.6 Hz, 1H), 8.50 (d, J=1.6 Hz, 1H), 8.06-7.97 (m, 2H), 7.68 (dd, J=1.6, 8.4 Hz, 1H), 7.60 (dd, J=1.6, 11.2 Hz, 1H), 7.43 (t, J=6.0 Hz, 1H), 7.41-7.32 (m, 1H), 4.20 (d, J=6.0 Hz, 2H), 3.44-3.18 (m, 2H), 2.49-2.43 (m, 6H), 1.81-1.69 (m, 2H), 1.60-1.50 (m, 4H), 1.41 (s, 9H), 1.40-1.29 (m, 2H). m/z: [ESI⁺] 566 (M+H)⁺.

### N-(3-(diethylamino)propyl)-2-(2-fluoro-4-formylphenyl)benzo[d]imidazo[2,1-b]thiazole-7-carboxamide

Compound N-(3-(diethylamino)propyl)-2-(2-fluoro-4-formylphenyl)benzo[d]imidazo[2,1-b]thiazole-7-carboxamide was prepared from 2-bromo-N-(3-(diethylamino)propyl)benzo[d]imidazo[2,1-b]thiazole-7-carboxamide (0.50 g, 1.22 mmol) and (2-fluoro-4-formylphenyl)boronic acid (0.40 g, 2.38 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as an off-white solid.

Yield 0.26 g (47%). ¹H NMR (400 MHz, CD₃OD) δ 9.94 (s, 1H), 8.59 (d, J=3.6 Hz, 1H), 8.52-8.47 (m, 2H), 8.36-8.33 (m, 1H), 8.03-8.00 (m, 1H), 7.79-7.73 (m, 1H), 7.69-7.62 (m, 1H), 3.57-3.52 (m, 2H), 3.30-3.20 (m, 6H), 2.14-2.05 (m, 2H), 1.38-1.29 (t, J=7.2 Hz, 6H). Amide NH proton not observed. m/z: [ESI⁺] 453 (M+H)⁺.

### 2-(2-fluoro-4-formylphenyl)-N-(3-(piperidin-1-yl)propyl)benzo[d]imidazo[2,1-b]thiazole-7-carboxamide

Compound 2-(2-fluoro-4-formylphenyl)-N-(3-(piperidin-1-yl)propyl)benzo[d]imidazo[2,1-b]thiazole-7-carboxamide was prepared from 2-bromo-N-(3-(piperidin-1-yl)propyl)benzo[d]imidazo[2,1-b]thiazole-7-carboxamide (800 mg, 1.899 mmol) and (2-fluoro-4-formylphenyl)boronic acid (638 mg, 3.799 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as an off-white solid.

Yield 220 mg (25%). ¹H NMR (400 MHz, DMSO) δ 10.02 (s, 1H), 8.94 (d, J=3.6 Hz, 1H), 8.68 (t, J=5.6 Hz, 1H), 8.51 (d, J=1.6 Hz, 1H), 8.45-8.35 (m, 1H), 8.33 (d, J=8.4 Hz, 1H), 8.03 (dd, J=1.6, 8.4 Hz, 1H), 7.93-7.80 (m, 2H), 3.36-3.29 (m, 2H), 2.45-2.38 (m, 6H), 1.76-1.70 (m, 2H), 1.55-1.50 (m, 4H), 1.43-1.38 (m, 2H). m/z: [ESI⁺] 465 (M+H)⁺.

### 2-(2-hydroxyphenyl)-N-(3-(piperidin-1-yl)propyl)benzo[d]imidazo[2,1-b]thiazole-7-carboxamide

Compound 2-(2-hydroxyphenyl)-N-(3-(piperidin-1-yl)propyl)benzo[d]imidazo[2,1-b]thiazole-7-carboxamide was prepared from 2-bromo-N-(3-(piperidin-1-yl)propyl)benzo[d]imidazo[2,1-b]thiazole-7-carboxamide (1.68 g, 3.99 mmol) and (2-hydroxyphenyl)boronic acid (1.38 g, 10.01 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as an off-white solid.

Yield 0.40 g (23%). ¹H NMR (400 MHz, DMSO) δ 10.59 (br s, 1H), 8.75 (s, 1H), 8.64 (t, J=5.6 Hz, 1H), 8.49 (d, J=1.6 Hz, 1H), 8.20 (d, J=8.4 Hz, 1H), 8.01 (dd, J=1.6, 8.4 Hz, 1H), 7.97 (dd, J=1.6, 8.0 Hz, 1H), 7.18-7.12 (m, 1H), 6.97 (dd, J=1.6, 8.0 Hz, 1H), 6.94-6.87 (m, 1H), 3.35-3.29 (m, 2H), 2.40-2.29 (m, 6H), 1.77-1.66 (m, 2H), 1.57-1.45 (m, 4H), 1.44-1.32 (m, 2H). m/z: [ESI⁺] 435 (M+H)⁺.

### 2-(4-(aminomethyl)-2-(trifluoromethyl)phenyl)-N-(3-(piperidin-1-yl)propyl)benzo[d]imidazo[2,1-b]thiazole-7-carboxamide formate (Compound 372)

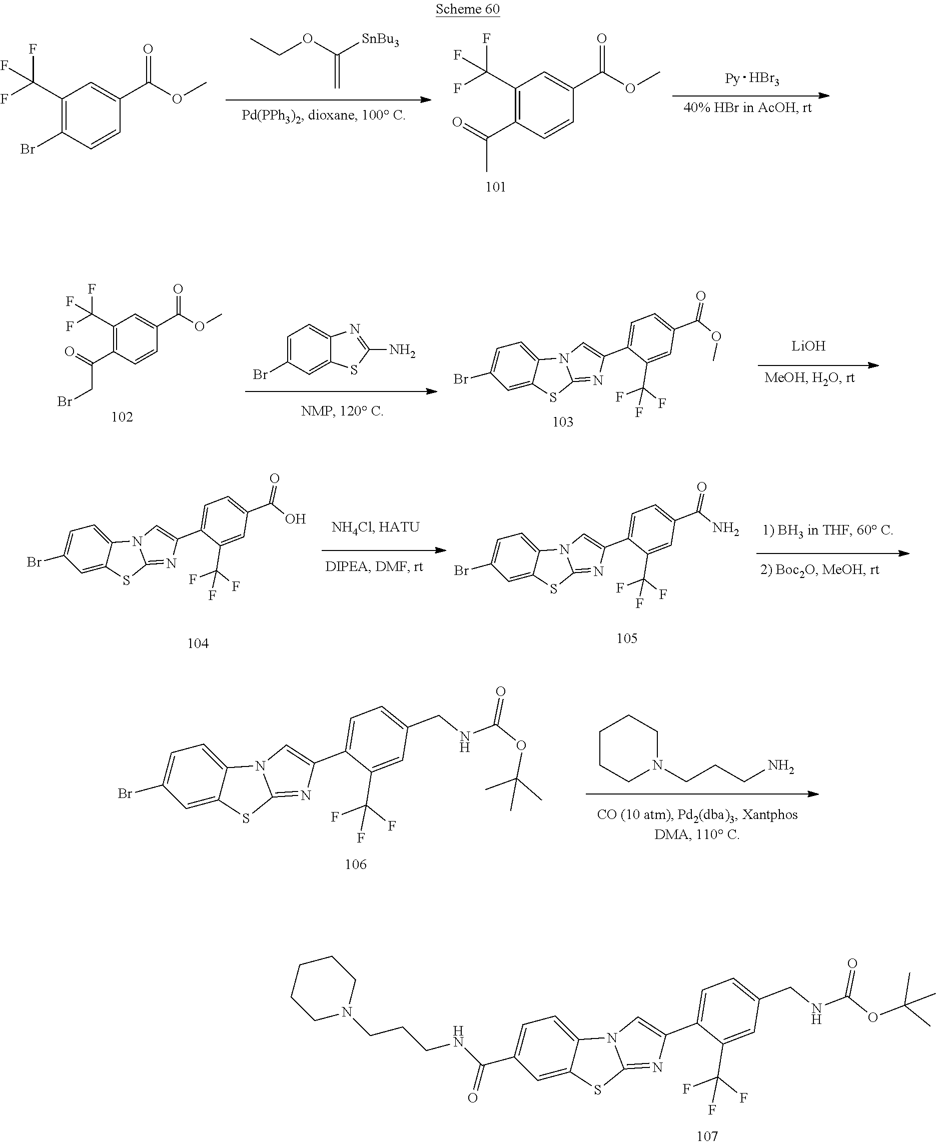

### methyl 4-acetyl-3-(trifluoromethyl)benzoate

To a stirred solution of methyl 4-bromo-3-(trifluoromethyl)benzoate (10.00 g, 35.33 mmol) and tributyl(1-ethoxyvinyl)stannane (38.28 g, 105.99 mmol) in 1,4-dioxane (100 mL) were added bis(triphenylphosphine)palladium (II) dichloride (3.72 g, 5.30 mmol) at room temperature under a nitrogen atmosphere. The resulting mixture was stirred for 16 h at 100° C. under a nitrogen atmosphere. The resulting mixture was cooled down to room temperature followed by the dropwise addition of aqueous HCl (6 M, 50 mL) at room temperature. The resulting mixture was stirred for additional 0.5 h at room temperature and was extracted with ethyl acetate (3×100 mL). The combined organic layers were washed with brine (200 mL) and dried over anhydrous sodium sulfate. After filtration, the filtrate was concentrated under reduced pressure. The residue was purified by silica gel column chromatography, eluting with 4% ethyl acetate in petroleum ether to afford methyl 4-acetyl-3-(trifluoromethyl)benzoate as a colorless oil.

Yield 8.50 g (98%). ¹H NMR (400 MHz, CDCl₃) δ 8.39 (d, J=1.6 Hz, 1H), 8.28 (dd, J=1.6, 8.0 Hz, 1H), 7.53 (d, J=8.0 Hz, 1H), 4.00 (s, 3H), 2.62 (s, 3H). no MS signal.

### methyl 4-(2-bromoacetyl)-3-(trifluoromethyl)benzoate

A solution of methyl 4-acetyl-3-(trifluoromethyl)benzoate (4.20 g, 17.06 mmol) and pyridinium tribromide (4.91 g, 15.35 mmol) in 40 wt. % hydrogen bromide solution in acetic acid(50 mL) was stirred for 16 h at room temperature under a nitrogen atmosphere. The resulting mixture was diluted with water (150 mL) and extracted with ethyl acetate (3×60 mL). The combined organic layers were washed with brine (100 mL) and dried over anhydrous sodium sulfate. After filtration, the filtrate was concentrated under reduced pressure. The residue was purified by silica gel column chromatography, eluting with 5% ethyl acetate in petroleum ether to afford methyl 4-(2-bromoacetyl)-3-(trifluoromethyl)benzoate as a yellow oil.

Yield 4.20 g (75%). ¹H NMR (400 MHz, CDCl₃) δ 8.43 (d, J=1.6 Hz, 1H), 8.32 (dd, J=1.6, 8.0 Hz, 1H), 7.61 (d, J=8.0 Hz, 1H), 4.38 (s, 2H), 4.01 (s, 3H). m/z: [ESI] 323, 325 (M−H)⁻.

### methyl 4-(7-bromobenzo[d]imidazo[2,1-b]thiazol-2-yl)-3-(trifluoromethyl)benzoate

A solution of methyl 4-(2-bromoacetyl)-3-(trifluoromethyl)benzoate (4.20 g, 12.92 mmol) and 6-bromobenzo[d]thiazol-2-amine (2.37 g, 10.34 mmol) in 1-methylpyrrolidin-2-one (40 mL) was stirred for 16 h at 120° C. under a nitrogen atmosphere. The resulting mixture was cooled to room temperature and purified by reverse phase flash chromatography with the flowing conditions: Column: WelFlash™ C18-I, 20-40 um, 330 g; Eluent A: water (plus 5 mmol/L ammonium bicarbonate); Eluent B: acetonitrile; Gradient: 50%-95% B in 30 min; Flow rate: 80 mL/min; Detector: UV 254/220 nm. The fractions containing the desired product were collected and concentrated under reduced pressure to afford methyl 4-(7-bromobenzo[d]imidazo[2,1-b]thiazol-2-yl)-3-(trifluoromethyl)benzoate as an off-white solid.

Yield 0.85 g (18%). ¹H NMR (400 MHz, DMSO) δ 8.65 (s, 1H), 8.37 (d, J=2.0 Hz, 1H), 8.31 (d, J=1.6 Hz, 1H), 8.28 (dd, J=2.0, 8.0 Hz, 1H), 8.17 (d, J=8.4 Hz, 1H), 8.11 (d, J=8.0 Hz, 1H), 7.77 (dd, J=1.6, 8.4 Hz, 1H), 3.93 (s, 3H). m/z: [ESI⁺] 455, 457 (M+H)⁺.

### 4-(7-bromobenzo[d]imidazo[2,1-b]thiazol-2-yl)-3-(trifluoromethyl)benzoic acid

To a stirred solution of methyl 4-(7-bromobenzo[d]imidazo[2,1-b]thiazol-2-yl)-3-(trifluoromethyl)benzoate (850 mg, 1.867 mmol) in methanol (10 mL) and water (3 mL) was added lithium hydroxide (224 mg, 9.353 mmol) at room temperature under a nitrogen atmosphere. The reaction solution was stirred for 16 h at room temperature. The resulting mixture was acidified to pH 6 with aqueous 2 M HCl. The precipitated solids were collected by filtration, washed with dichloromethane (3×10 mL) and dried, to afford 4-(7-bromobenzo[d]imidazo[2,1-b]thiazol-2-yl)-3-(trifluoromethyl)benzoic acid as an off-white solid.

Yield 430 mg (52%). ¹H NMR (400 MHz, DMSO) δ 13.50 (br s, 1H), 8.64 (s, 1H), 8.38 (d, J=2.0 Hz, 1H), 8.31 (d, J=1.6 Hz, 1H), 8.27 (dd, J=2.0, 8.0 Hz, 1H), 8.18 (d, J=8.4 Hz, 1H), 8.08 (d, J=8.0 Hz, 1H), 7.77 (dd, J=1.6, 8.4 Hz, 1H). m/z: [ESI⁺] 441, 443 (M+H)⁺.

### 4-(7-bromobenzo[d]imidazo[2,1-b]thiazol-2-yl)-3-(trifluoromethyl)benzamide

Compound 4-(7-bromobenzo[d]imidazo[2,1-b]thiazol-2-yl)-3-(trifluoromethyl)benzamide was prepared from 4-(7-bromobenzo[d]imidazo[2,1-b]thiazol-2-yl)-3-(trifluoromethyl)benzoic acid (950 mg, 2.150 mmol) and ammonium chloride (230 mg, 4.300 mmol) following a similar procedure to that described for the synthesis of 2-bromo-N-(3-(piperidin-1-yl)propyl)benzo[d]imidazo[2,1-b]thiazole-7-carboxamide, and was isolated as a white solid.

Yield 580 mg (61%). ¹H NMR (400 MHz, DMSO) δ 8.61 (s, 1H), 8.38 (d, J=2.0 Hz, 1H), 8.33 (d, J=1.6 Hz, 1H), 8.28 (br s, 1H), 8.23 (dd, J=2.0, 8.0 Hz, 1H), 8.17 (d, J=8.4 Hz, 1H), 8.01 (d, J=8.0 Hz, 1H), 7.78 (dd, J=1.6, 8.4 Hz, 1H), 7.62 (br s, 1H). m/z: [ESI⁺] 440, 442 (M+H)⁺.

### tert-butyl (4-(7-bromobenzo[d]imidazo[2,1-b]thiazol-2-yl)-3-(trifluoromethyl)benzyl)carbamate

Compound tert-butyl (4-(7-bromobenzo[d]imidazo[2,1-b]thiazol-2-yl)-3-(trifluoromethyl)benzyl)carbamate was prepared from 4-(7-bromobenzo[d]imidazo[2,1-b]thiazol-2-yl)-3-(trifluoromethyl)benzamide (580 mg, 1.317 mmol) following a similar procedure to that described for the synthesis of tert-butyl (4-bromo-3-cyclopropylbenzyl)carbamate, and was isolated as a light yellow solid.

Yield 300 mg (43%). m/z: [ESI⁺] 526, 528 (M+H)⁺.

### tert-butyl (4-(7-((3-(piperidin-1-yl)propyl)carbamoyl)benzo[d]imidazo[2,1-b]thiazol-2-yl)-3-(trifluoromethyl)benzyl)carbamate

To a solution of tert-butyl (4-(7-bromobenzo[d]imidazo[2,1-b]thiazol-2-yl)-3-(trifluoromethyl)benzyl)carbamate (300 mg, 0.571 mmol) and 3-(piperidin-1-yl)propan-1-amine (97 mg, 0.686 mmol) in N,N-dimethylacetamide (6 mL) were added tris(dibenzylideneacetone)dipalladium (0) (33 mg, 0.036 mmol) and 4,5-bis(diphenylphosphino)-9,9-dimethylxanthene (66 mg, 0.114 mmol) in a pressure vessel. The mixture was stirred at 110° C. for 4 h under a carbon monoxide atmosphere (10 atm.). The reaction mixture was cooled to room temperature and was purified by reverse phase flash chromatography with the following conditions: Column: WelFlash™ C18-I, 20-40 um, 120 g; Eluent A: water (plus 10 mmol/L ammonium bicarbonate); Eluent B: acetonitrile; Gradient: 25%-55% B in 25 min; Flow rate: 60 mL/min; Detector: UV 254/220 nm. The fractions containing the desired product were collected and concentrated under reduced pressure to afford tert-butyl (4-(7-((3-(piperidin-1-yl)propyl)carbamoyl)benzo[d]imidazo[2,1-b]thiazol-2-yl)-3-(trifluoromethyl)benzyl)carbamate as a light yellow solid.

Yield 200 mg (57%). ¹H NMR (400 MHz, CDCl₃) δ 8.55 (t, J=5.6 Hz, 1H), 8.50 (s, 1H), 8.37 (s, 1H), 8.21 (d, J=8.4 Hz, 1H), 7.98-7.95 (m, 2H), 7.80-7.65 (m, 1H), 7.56 (d, J=8.4 Hz, 1H), 5.03 (br s, 1H), 4.45-4.42 (m, 2H), 3.68-3.64 (m, 2H), 3.16-3.11 (m, 2H), 2.25-2.19 (m, 2H), 2.01-1.93 (m, 8H), 1.50 (s, 9H), 1.35-1.28 (m, 2H). m/z: [ESI⁺] 616 (M+H)⁺.

### 4-(7-chloroimidazo[2′,1′:2,3]thiazolo[4,5-c]pyridin-2-yl)-N-methylbenzamide

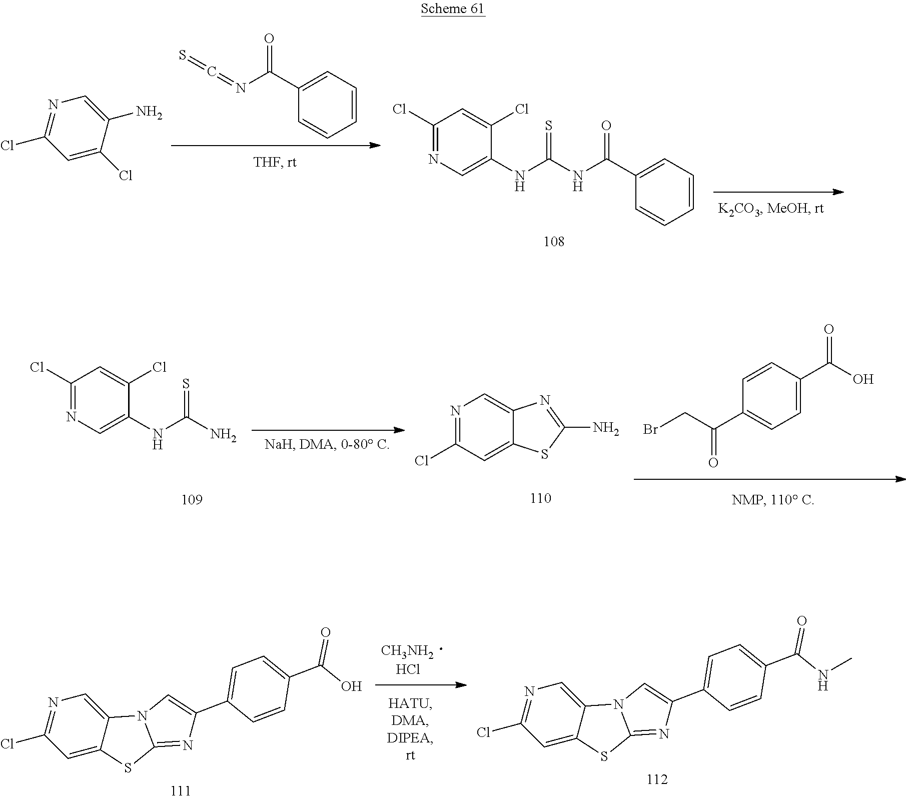

### N-((4,6-dichloropyridin-3-yl)carbamothioyl)benzamide

To a stirred solution of 4,6-dichloropyridin-3-amine (3.00 g, 18.40 mmol) in tetrahydrofuran (30 mL) was added benzoyl isothiocyanate (4.51 g, 27.63 mmol) dropwise at 0° C. The resulting solution was stirred for 16 h at room temperature under a nitrogen atmosphere. The product was precipitated by the addition of petroleum ether (100 mL). After filtration, the filter cake was washed with petroleum ether (3×10 mL) and oven dried to afford N-((4,6-dichloropyridin-3-yl)carbamothioyl)benzamide as a yellow solid.

Yield 5.75 g (96%). ¹H NMR (300 MHz, DMSO) δ 12.40 (br s, 1H), 12.01 (br s, 1H), 8.76 (s, 1H), 8.06-8.02 (m, 1H), 8.02-7.96 (m, 2H), 7.78-7.65 (m, 1H), 7.59-7.54 (m, 2H). m/z: [ESI⁺] 326, 328 (M+H)⁺.

### 1-(4,6-dichloropyridin-3-yl)thiourea

To a stirred solution of N-((4,6-dichloropyridin-3-yl)carbamothioyl)benzamide (16.40 g, 50.28 mmol) in methanol (200 mL) was added potassium carbonate (6.95 g, 50.28 mmol) in portion-wise at 0° C. The resulting mixture was stirred for 16 h at room temperature under a nitrogen atmosphere. The resulting mixture was diluted with water (200 mL). The precipitated solids were collected by filtration and washed with water (3×50 mL). The resulting solid was oven dried to afford 1-(4,6-dichloropyridin-3-yl)thiourea as a white solid.

Yield 10.23 g (92%). ¹H NMR (400 MHz, DMSO) δ 9.48 (br s, 1H), 8.55 (s, 1H), 8.15 (br s, 1H), 7.86 (s, 1H), 7.50 (br s, 1H). m/z: [ESI⁺] 222, 224 (M+H)⁺.

### 6-chlorothiazolo[4,5-c]pyridin-2-amine

To a stirred solution of 1-(4,6-dichloropyridin-3-yl)thiourea (3.00 g, 13.51 mmol) in N,N-dimethylacetamide (30 mL) was added sodium hydride (60% dispersion in mineral oil, 0.97 g, 24.25 mmol) portion-wise at 0° C. The resulting mixture was stirred for 15 min at room temperature and then stirred for an additional 3 h at 80° C. under a nitrogen atmosphere. The resulting solution was cooled to 0° C. and quenched with saturated aqueous ammonium chloride (30 mL). The precipitated solids were filtered. The filter cake was washed with water (3×5 mL) and oven dried to afford 6-chlorothiazolo[4,5-c]pyridin-2-amine as a grey solid.

Yield 2.30 g (92%). ¹H NMR (400 MHz, DMSO) δ 8.33 (s, 1H), 7.93 (br s, 2H), 7.88 (s, 1H). m/z: [ESI⁺] 186, 188 (M+H)⁺.

### 4-(7-chloroimidazo[2′,1′:2,3]thiazolo[4,5-c]pyridin-2-yl)benzoic acid

Compound 4-(7-chloroimidazo[2′,1′:2,3]thiazolo[4,5-c]pyridin-2-yl)benzoic acid was prepared from 6-chlorothiazolo[4,5-c]pyridin-2-amine (5.00 g, 26.93 mmol) and 4-(2-bromoacetyl)benzoic acid (6.55 g, 26.95 mmol) following a similar procedure to that described for the synthesis of methyl 4-(7-bromobenzo[d]imidazo[2,1-b]thiazol-2-yl)-3-(trifluoromethyl)benzoate, and was isolated as a red solid.

Yield 8.00 g (90%). ¹H NMR (400 MHz, DMSO) δ 13.00 (br s, 1H), 9.06 (s, 1H), 8.96 (s, 1H), 8.31 (s, 1H), 8.01 (d, J=8.4 Hz, 2H), 7.95 (d, J=8.4 Hz, 2H). m/z: [ESI⁺] 330, 332 (M+H)⁺.

### 4-(7-chloroimidazo[2′,7′:2,3]thiazolo[4,5-c]pyridin-2-yl)-N-methylbenzamide

Compound 4-(7-chloroimidazo[2′,1′:2,3]thiazolo[4,5-c]pyridin-2-yl)-N-methylbenzamide was prepared from 4-(7-chloroimidazo[2′,1′:2,3]thiazolo[4,5-c]pyridin-2-yl)benzoic acid (1.80 g, 5.45 mmol) and methylamine hydrochloride (0.74 g, 10.92 mmol) following a similar procedure to that described for the synthesis of 2-bromo-N-(3-(piperidin-1-yl)propyl)benzo[d]imidazo[2,1-b]thiazole-7-carboxamide, and was isolated as a pink solid.

Yield 1.26 g (67%). ¹H NMR (400 MHz, DMSO) δ 9.07 (s, 1H), 8.95 (s, 1H), 8.44 (q, J=4.8 Hz, 1H), 8.35 (s, 1H), 7.94-7.90 (m, 4H), 2.81 (d, J=4.8 Hz, 3H). m/z: [ESI⁺] 343, 345 (M+H)⁺.

### N-(3-aminopropyl)-2-(2-fluoro-4-(methylcarbamoyl)phenyl)benzo[d]imidazo[2,1-b]thiazole-7-carboxamide dihydrochloride

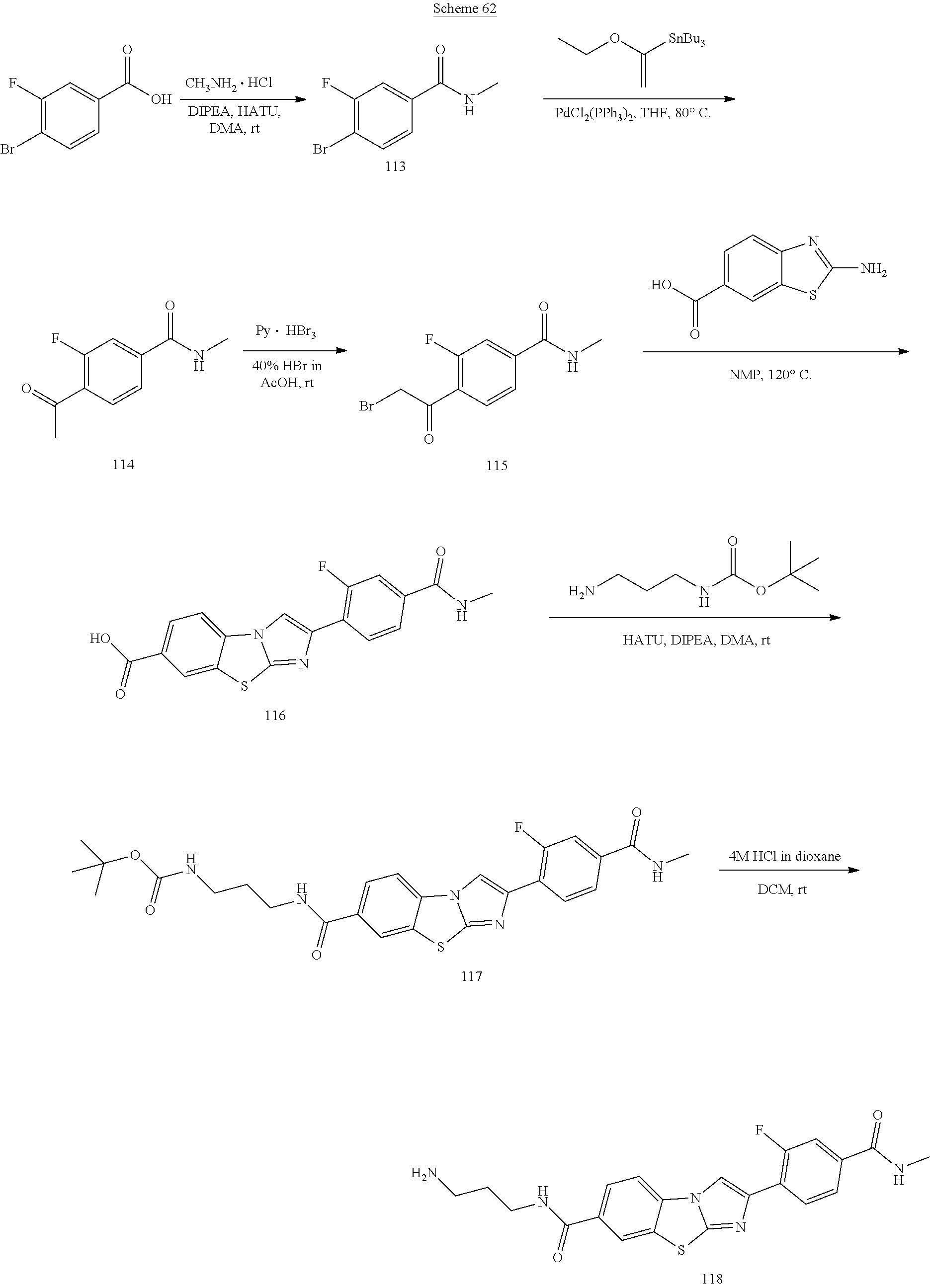

### 4-bromo-3-fluoro-N-methylbenzamide

Compound 4-bromo-3-fluoro-N-methylbenzamide was prepared from 4-bromo-3-fluorobenzoic acid (20.00 g, 91.32 mmol) and methylamine hydrochloride (8.50 g, 125.89 mmol) following a similar procedure to that described for the synthesis of 2-bromo-N-(3-(piperidin-1-yl)propyl)benzo[d]imidazo[2,1-b]thiazole-7-carboxamide, and was isolated as an off-white solid.

Yield 13.00 g (61%). ¹H NMR (400 MHz, CDCl₃) δ 7.61 (dd, J=6.8, 8.4 Hz, 1H), 7.57 (dd, J=2.0, 9.2 Hz, 1H), 7.43 (dd, J=2.0, 8.4 Hz, 1H), 6.59 (q, J=4.8 Hz, 1H), 3.01 (d, J=4.8 Hz, 3H). m/z: [ESI⁺] 232, 234 (M+H)⁺.

### 4-acetyl-3-fluoro-N-methylbenzamide

Compound 4-acetyl-3-fluoro-N-methylbenzamide was prepared from 4-bromo-3-fluoro-N-methylbenzamide (13.00 g, 56.02 mmol), following a similar procedure to that described for the synthesis of methyl 4-acetyl-3-(trifluoromethyl)benzoate, and was isolated as an off-white solid.

Yield 10.33 g (94%). ¹H NMR (400 MHz, CDCl₃) δ 7.93 (dd, J=7.2, 8.0 Hz, 1H), 7.62 (dd, J=1.6, 11.2 Hz, 1H), 7.56 (dd, J=1.6, 8.0 Hz, 1H), 6.34 (q, J=4.8 Hz, 1H), 3.05 (d, J=4.8 Hz, 3H), 2.68 (d, J=4.8 Hz, 3H). m/z: [ESI⁺] 196 (M+H)⁺.

### 4-(2-bromoacetyl)-3-fluoro-N-methylbenzamide

Compound 4-(2-bromoacetyl)-3-fluoro-N-methylbenzamide was prepared from 4-acetyl-3-fluoro-N-methylbenzamide (1.60 g, 8.20 mmol) following a similar procedure to that described for the synthesis of methyl 4-(2-bromoacetyl)-3-(trifluoromethyl)benzoate, and was isolated as a yellow solid.

Yield 1.50 g (67%). ¹H NMR (400 MHz, CDCl₃) δ 8.10-7.93 (m, 1H), 7.71-7.56 (m, 2H), 6.25 (q, J=4.8 Hz, 1H), 4.54 (d, J=2.4 Hz, 2H), 3.07 (d, J=4.8 Hz, 3H). m/z: [ESI⁺] 274, 276 (M+H)⁺.

### 2-(2-fluoro-4-(methylcarbamoyl)phenyl)benzo[d]imidazo[2,]-b]thiazole-7-carboxylic acid

Compound 2-(2-fluoro-4-(methylcarbamoyl)phenyl)benzo[d]imidazo[2,1-b]thiazole-7-carboxylic acid was prepared from 4-(2-bromoacetyl)-3-fluoro-N-methylbenzamide (2.54 g, 9.27 mmol) and 2-aminobenzo[d]thiazole-6-carboxylic acid (1.50 g, 7.72 mmol) following a similar procedure to that described for the synthesis of methyl 4-(7-bromobenzo[d]imidazo[2,1-b]thiazol-2-yl)-3-(trifluoromethyl)benzoate, and was isolated as a brown solid.

Yield 1.38 g (48%). ¹H NMR (400 MHz, DMSO) δ 8.86-8.78 (m, 1H), 8.65 (d, J=1.6 Hz, 1H), 8.58 (q, J=4.4 Hz, 1H), 8.30-8.19 (m, 2H), 8.11 (dd, J=1.6, 8.4 Hz, 1H), 7.83-7.75 (m, 2H), 2.81 (d, J=4.4 Hz, 3H). OH proton not observed. m/z: [ESI⁺] 370 (M+H)⁺.

### tert-butyl (3-(2-(2-fluoro-4-(methylcarbamoyl)phenyl)benzo[d]imidazo[2,1-b]thiazole-7-carboxamido)propyl)carbamate

Compound tert-butyl (3-(2-(2-fluoro-4-(methylcarbamoyl)phenyl)benzo[d]imidazo[2,1-b]thiazole-7-carboxamido)propyl)carbamate was prepared from 2-(2-fluoro-4-(methylcarbamoyl)phenyl)benzo[d]imidazo[2,1-b]thiazole-7-carboxylic acid (1.00 g, 2.71 mmol) and tert-butyl (3-aminopropyl)carbamate (1.00 g, 5.74 mmol) following a similar procedure to that described for the synthesis of 2-bromo-N-(3-(piperidin-1-yl)propyl)benzo[d]imidazo[2,1-b]thiazole-7-carboxamide, and was isolated as a dark yellow solid.

Yield 1.00 g (70%). ¹H NMR (400 MHz, DMSO) δ 8.82 (d, J=3.6 Hz, 1H), 8.59-8.52 (m, 2H), 8.50 (d, J=1.6 Hz, 1H), 8.29 (d, J=8.4 Hz, 1H), 8.27-8.16 (m, 1H), 8.02 (dd, J=1.6, 8.4 Hz, 1H), 7.83-7.73 (m, 2H), 6.83 (t, J=5.6 Hz, 1H), 3.32-3.28 (m, 2H), 3.03-2.99 (m, 2H), 2.82 (d, J=4.4 Hz, 3H), 1.69-1.65 (m, 2H), 1.39 (s, 9H). m/z: [ESI⁺] 526 (M+H)⁺.

### N-(3-aminopropyl)-2-(2-fluoro-4-(methylcarbamoyl)phenyl)benzo[d]imidazo[2,1-b]thiazole-7-carboxamide hydrochloride

Compound N-(3-aminopropyl)-2-(2-fluoro-4-(methylcarbamoyl)phenyl)benzo[d]imidazo[2,1-b]thiazole-7-carboxamide hydrochloride was prepared from tert-butyl (3-(2-(2-fluoro-4-(methylcarbamoyl)phenyl)benzo[d]imidazo[2,1-b]thiazole-7-carboxamido)propyl)carbamate (600 mg, 1.143 mmol) following a similar procedure to that described for the synthesis of 4-amino-1-(4-bromo-3-fluorophenyl)butan-1-one hydrochloride, and was isolated as a yellow solid.

Yield 480 mg (91%). m/z: [ESI⁺] 426 (M+H)⁺.

### (S)—N-(pyrrolidin-3-ylmethyl)-2-(p-tolyl)benzo[d]imidazo[2,1-b]thiazole-7-carboxamide hydrochloride (Compound 116)

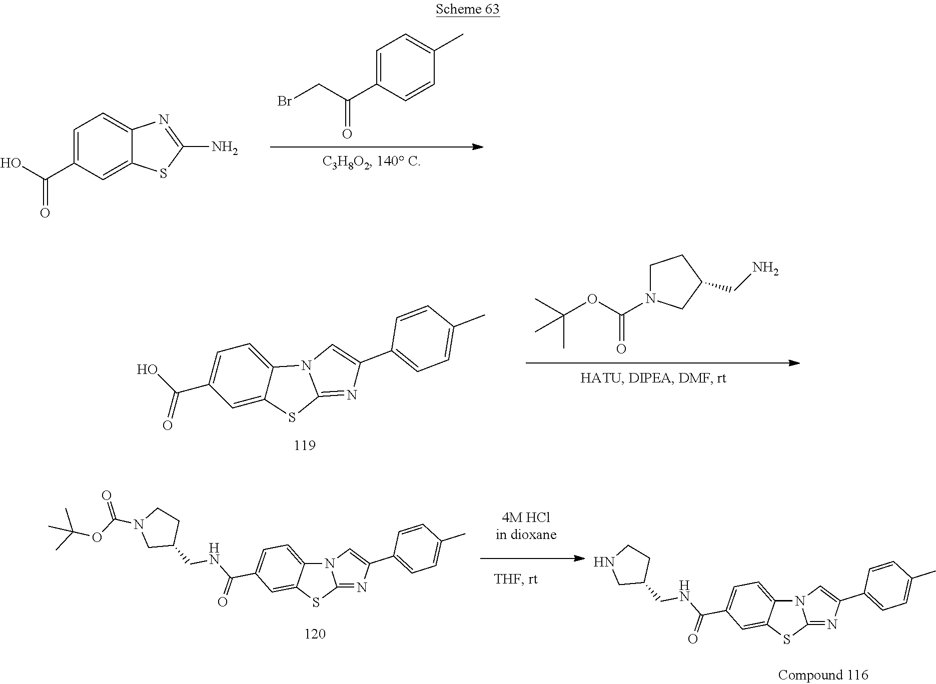 [SMILES: Nc1nc2ccc(C(=O)O)cc2s1 ; Cc1ccc(C(=O)CBr)cc1 ; Cc1ccc(-c2cn3c(n2)sc2cc(C(=O)O)ccc23)cc1 ; CC(C)(C)OC(=O)N1CC[C@H](CN)C1 ; Cc1ccc(-c2cn3c(n2)sc2cc(C(=O)NC[C@H]4CCN(C(=O)OC(C)(C)C)C4)ccc23)cc1 ; Cc1ccc(-c2cn3c(n2)sc2cc(C(=O)NC[C@H]4CCNC4)ccc23)cc1]

### 2-(p-tolyl)benzo[d]imidazo[2,1-b]thiazole-7-carboxylic acid

To a stirred solution of 2-amino-1,3-benzothiazole-6-carboxylic acid (15.00 g, 77.24 mmol) in 2-methoxyethan-1-ol (150 mL) was added 2-bromo-1-(4-methylphenyl)ethanone (18.10 g, 84.95 mmol) portion-wise at room temperature. The reaction mixture was stirred for 16 h at 140° C. The resulting mixture was cooled to room temperature. The precipitated solids were collected by filtration and washed with ethyl ether (3×60 mL) to afford 2-(p-tolyl)benzo[d]imidazo[2,1-b]thiazole-7-carboxylic acid as a white solid.

Yield 9.50 g (40%). ¹H NMR (400 MHz, DMSO) δ 13.10 (br s, 1H), 8.78 (s, 1H), 8.66 (d, J=1.6 Hz, 1H), 8.13 (dd, J=1.6, 8.4 Hz, 1H), 8.05 (d, J=8.4 Hz, 1H), 7.82-7.74 (d, J=8.0 Hz, 2H), 7.26 (d, J=8.0 Hz, 2H), 2.34 (s, 3H). m/z: [ESI⁺] 309 (M+H)⁺.

### tert-butyl (R)-3-((2-(p-tolyl)benzo[d]imidazo[2,1-b]thiazole-7-carboxamido)methyl)pyrrolidine-1-carboxylate

Compound tert-butyl (R)-3-((2-(p-tolyl)benzo[d]imidazo[2,1-b]thiazole-7-carboxamido)methyl)pyrrolidine-1-carboxylate was prepared from 2-(p-tolyl)benzo[d]imidazo[2,1-b]thiazole-7-carboxylic acid (300 mg, 0.973 mmol) and tert-butyl (R)-3-(aminomethyl)pyrrolidine-1-carboxylate (205 mg, 1.024 mmol) following a similar procedure to that described for the synthesis of 2-bromo-N-(3-(piperidin-1-yl)propyl)benzo[d]imidazo[2,1-b]thiazole-7-carboxamide, and was isolated as a dark yellow solid.

Yield 370 mg (78%). ¹H NMR (300 MHz, CDCl₃) δ 8.16 (d, J=1.6 Hz, 1H), 7.95 (s, 1H), 7.84 (d, J=8.4 Hz, 1H), 7.77 (d, J=8.0 Hz, 2H), 7.62 (d, J=8.4 Hz, 1H), 7.26 (d, J=8.0 Hz, 2H), 6.60-6.52 (m, 1H), 3.76-3.44 (m, 4H), 3.44-3.29 (m, 1H), 3.29-3.02 (m, 1H), 2.66-2.51 (m, 1H), 2.41 (s, 3H), 2.16-2.03 (m, 1H), 1.84-1.63 (m, 1H), 1.49 (s, 9H). m/z: [ESI⁺] 491 (M+H)⁺.

### (S)—N-(pyrrolidin-3-ylmethyl)-2-(p-tolyl)benzo[d]imidazo[2,1-b]thiazole-7-carboxamide hydrochloride (Compound 116)

Compound (S)—N-(pyrrolidin-3-ylmethyl)-2-(p-tolyl)benzo[d]imidazo[2,1-b]thiazole-7-carboxamide hydrochloride was prepared from tert-butyl (R)-3-((2-(p-tolyl)benzo[d]imidazo[2,1-b]thiazole-7-carboxamido)methyl)pyrrolidine-1-carboxylate (200 mg, 0.408 mmol) following a similar procedure to that described for the synthesis of 4-amino-i-(4-bromo-3-fluorophenyl)butan-1-one hydrochloride, and was isolated as a light yellow solid.

Yield 150 mg (86%). ¹H NMR (400 MHz, DMSO) δ 9.37 (br s, 2H, NH₂+), 8.98 (t, J=5.6 Hz, 1H), 8.91 (s, 1H), 8.62 (s, 1H), 8.13 (s, 2H), 7.78 (d, J=8.0 Hz, 2H), 7.28 (d, J=8.0 Hz, 2H), 3.47-3.34 (m, 2H), 3.34-3.18 (m, 2H), 3.17-3.08 (m, 1H), 3.01-2.90 (m, 1H), 2.64-2.54 (m, 1H), 2.34 (s, 3H), 2.09-1.98 (m, 1H), 1.79-1.65 (m, 1H). m/z: [ESI⁺] 391 (M+H)⁺.

### (R)—N-(pyrrolidin-3-ylmethyl)-2-(p-tolyl)benzo[d]imidazo[2,1-b]thiazole-7-carboxamide hydrochloride (Compound 114)

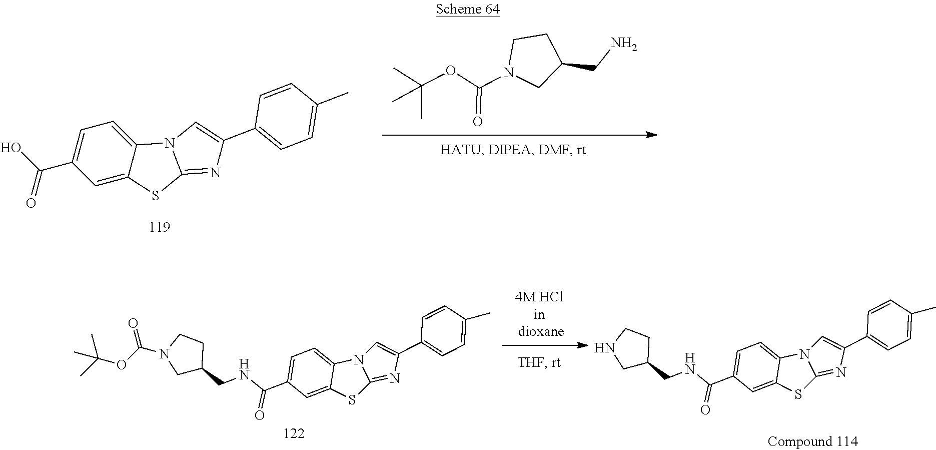 [SMILES: Cc1ccc(-c2cn3c(n2)sc2cc(C(=O)O)ccc23)cc1 ; CC(C)(C)OC(=O)N1CC[C@@H](CN)C1 ; Cc1ccc(-c2cn3c(n2)sc2cc(C(=O)NC[C@@H]4CCN(C(=O)OC(C)(C)C)C4)ccc23)cc1 ; Cc1ccc(-c2cn3c(n2)sc2cc(C(=O)NC[C@@H]4CCNC4)ccc23)cc1]

### tert-butyl (S)-3-((2-(p-tolyl)benzo[d]imidazo[2,1-b]thiazole-7-carboxamido)methyl)pyrrolidine-1-carboxylate

Compound tert-butyl (S)-3-((2-(p-tolyl)benzo[d]imidazo[2,1-b]thiazole-7-carboxamido)methyl)pyrrolidine-1-carboxylate was prepared from 2-(p-tolyl)benzo[d]imidazo[2,1-b]thiazole-7-carboxylic acid (300 mg, 0.973 mmol) and tert-butyl (S)-3-(aminomethyl)pyrrolidine-1-carboxylate (205 mg, 1.024 mmol) following a similar procedure to that described for the synthesis of 2-bromo-N-(3-(piperidin-1-yl)propyl)benzo[d]imidazo[2,1-b]thiazole-7-carboxamide, and was isolated as a dark yellow solid.

Yield 313 mg (66%). ¹H NMR (300 MHz, CDCl₃) δ 8.16 (d, J=1.6 Hz, 1H), 7.95 (s, 1H), 7.84 (d, J=8.4 Hz, 1H), 7.77 (d, J=8.0 Hz, 2H), 7.62 (d, J=8.4 Hz, 1H), 7.26 (d, J=8.0 Hz, 2H), 6.60-6.52 (m, 1H), 3.76-3.44 (m, 4H), 3.44-3.29 (m, 1H), 3.29-3.02 (m, 1H), 2.66-2.51 (m, 1H), 2.41 (s, 3H), 2.16-2.03 (m, 1H), 1.84-1.63 (m, 1H), 1.49 (s, 9H). m/z: [ESI⁺] 491 (M+H)⁺.

### (R)—N-(pyrrolidin-3-ylmethyl)-2-(p-tolyl)benzo[d]imidazo[2,1-b]thiazole-7-carboxamide hydrochloride (Compound 114)

Compound (R)—N-(pyrrolidin-3-ylmethyl)-2-(p-tolyl)benzo[d]imidazo[2,1-b]thiazole-7-carboxamide hydrochloride was prepared from tert-butyl (S)-3-((2-(p-tolyl)benzo[d]imidazo[2,1-b]thiazole-7-carboxamido)methyl)pyrrolidine-1-carboxylate (313 mg, 0.638 mmol) following a similar procedure to that described for the synthesis of 4-amino-i-(4-bromo-3-fluorophenyl)butan-1-one hydrochloride, and was isolated as a light yellow solid.

Yield 260 mg (95%). ¹H NMR (400 MHz, DMSO) δ 9.37 (br s, 2H, NH₂+), 8.98 (t, J=5.6 Hz, 1H), 8.91 (s, 1H), 8.62 (s, 1H), 8.13 (s, 2H), 7.78 (d, J=8.0 Hz, 2H), 7.28 (d, J=8.0 Hz, 2H), 3.47-3.34 (m, 2H), 3.34-3.18 (m, 2H), 3.17-3.08 (m, 1H), 3.01-2.90 (m, 1H), 2.64-2.54 (m, 1H), 2.34 (s, 3H), 2.09-1.98 (m, 1H), 1.79-1.65 (m, 1H). m/z: [ESI⁺] 391 (M+H)⁺.

### N-(3-(ethylamino)propyl)-2-(4-(methylcarbamoyl)phenyl)benzo[d]imidazo[2,1-b]thiazole-7-carboxamide 2,2,2-trifluoroacetate (Compound 229)

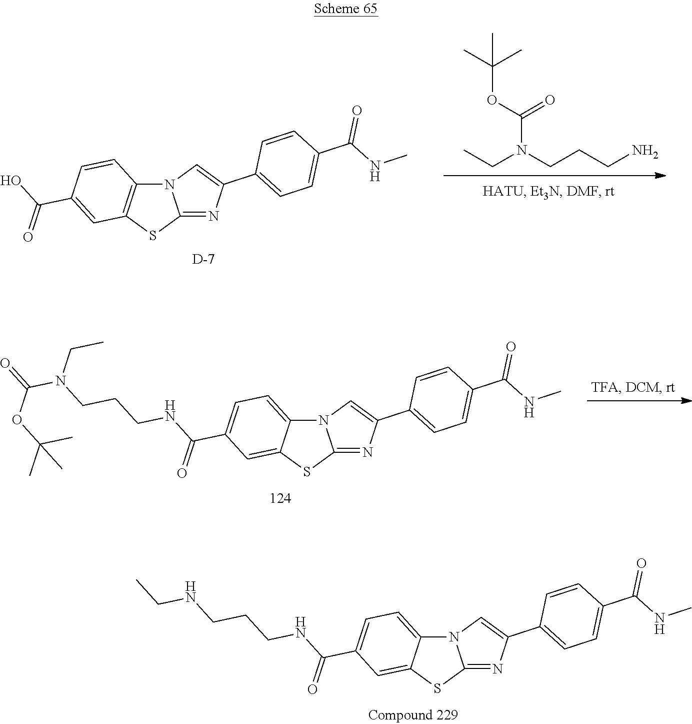

### tert-butyl ethyl(3-(2-(4-(methylcarbamoyl)phenyl)benzo[d]imidazo[2,1-b]thiazole-7-carboxamido)propyl)carbamate

Compound tert-butyl ethyl(3-(2-(4-(methylcarbamoyl)phenyl)benzo[d]imidazo[2,1-b]thiazole-7-carboxamido)propyl)carbamate was prepared from 2-(4-(methylcarbamoyl)phenyl)benzo[d]imidazo[2,1-b]thiazole-7-carboxylic acid (400 mg, 1.138 mmol) and tert-butyl (3-aminopropyl)(ethyl)carbamate (300 mg, 1.483 mmol) following a similar procedure to that described for the synthesis of 2-bromo-N-(3-(piperidin-1-yl)propyl)benzo[d]imidazo[2,1-b]thiazole-7-carboxamide, and was isolated as a yellow solid.

Yield 200 mg (33%). ¹H NMR (300 MHz, DMSO) δ 8.92 (s, 1H), 8.73 (t, J=5.6 Hz, 0.5H), 8.57 (d, J=5.6 Hz, 0.5H), 8.49 (s, 1H), 8.44 (q, J=4.4 Hz, 1H), 8.09-8.00 (m, 2H), 7.98-7.87 (m, 4H), 3.37-3.10 (m, 4H), 2.80 (d, J=4.4 Hz, 3H), 2.70-2.58 (m, 2H), 1.82-1.65 (m, 2H), 1.38 (s, 4.5H), 1.10 (s, 4.5H), 1.05 (t, J=7.2 Hz, 3H). m/z: [ESI⁺] 536 (M+H)⁺.

### N-(3-(ethylamino)propyl)-2-(4-(methylcarbamoyl)phenyl)benzo[d]imidazo[2,1-b]thiazole-7-carboxamide 2,2,2-trifluoroacetate (Compound 229)

A solution of tert-butyl ethyl(3-(2-(4-(methylcarbamoyl)phenyl)benzo[d]imidazo[2,1-b]thiazole-7-carboxamido)propyl)carbamate (200 mg, 0.373 mmol) in dichloromethane (5 mL) and trifluoroacetic acid (1 mL) was stirred for 1 h at room temperature. The resulting mixture was concentrated under reduced pressure. The residue was triturated with petroleum ether/dichloromathane (20:1, 21 mL), filtered and oven dried to afford N-(3-(ethylamino)propyl)-2-(4-(methylcarbamoyl)phenyl)benzo[d]imidazo[2,1-b]thiazole-7-carboxamide 2,2,2-trifluoroacetate as a light yellow solid.

Yield 120 mg (58%). ¹H NMR (300 MHz, DMSO) δ 8.91 (s, 1H), 8.72 (t, J=5.6 Hz, 1H), 8.48 (s, 1H), 8.43 (q, J=4.4 Hz, 1H), 8.08-7.98 (m, 2H), 7.98-7.87 (m, 4H), 3.41-3.27 (m, 2H), 2.80 (d, J=4.4 Hz, 3H), 2.65-2.52 (m, 4H), 1.75-1.63 (m, 2H), 1.02 (t, J=7.2 Hz, 3H). Aliphatic NH proton not observed. m/z: [ESI⁺] 436 (M+H)⁺.

### tert-butyl 4-(2-(2-fluoro-4-(methylcarbamoyl)phenyl)benzo[d]imidazo[2,1-b]thiazole-7-carboxamido)piperidine-1-carboxylate

Compound tert-butyl 4-(2-(2-fluoro-4-(methylcarbamoyl)phenyl)benzo[d]imidazo[2,1-b]thiazole-7-carboxamido)piperidine-1-carboxylate was prepared from 2-(2-fluoro-4-(methylcarbamoyl)phenyl)benzo[d]imidazo[2,1-b]thiazole-7-carboxylic acid (250 mg, 0.677 mmol) and tert-butyl 4-aminopiperidine-1-carboxylate (177 mg, 0.884 mmol) following a similar procedure to that described for the synthesis of 2-bromo-N-(3-(piperidin-1-yl)propyl)benzo[d]imidazo[2,1-b]thiazole-7-carboxamide, and was isolated as an off-white solid.

Yield 273 mg (73%). ¹H NMR (400 MHz, DMSO) δ 8.83 (d, J=3.6 Hz, 1H), 8.57 (q, J=4.4 Hz, 1H), 8.51 (d, J=1.6 Hz, 1H), 8.43 (d, J=7.6 Hz, 1H), 8.29 (d, J=8.4 Hz, 1H), 8.23 (t, J=8.0 Hz, 1H), 8.04 (dd, J=1.6, 8.4 Hz, 1H), 7.82-7.75 (m, 2H), 4.08-3.90 (m, 3H), 2.95-2.86 (m, 2H), 2.81 (d, J=4.4 Hz, 3H), 1.88-1.77 (m, 2H), 1.49-1.38 (m, 11H). m/z: [ESI⁺] 552 (M+H)⁺.

### tert-butyl 4-((2-(2-fluoro-4-(methylcarbamoyl)phenyl)benzo[d]imidazo[2,1-b]thiazol-7-yl)carbamoyl)piperidine-1-carboxylate

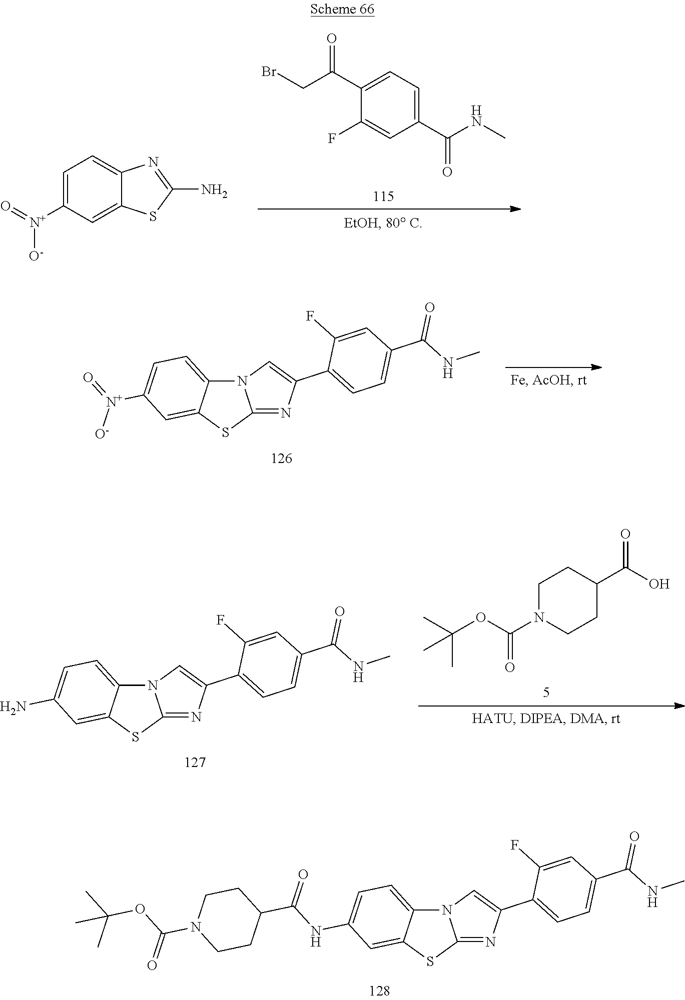

### 3-fluoro-N-methyl-4-(7-nitrobenzo[d]imidazo[2,1-b]thiazol-2-yl)benzamide

Compound 3-fluoro-N-methyl-4-(7-nitrobenzo[d]imidazo[2,1-b]thiazol-2-yl)benzamide was prepared from 6-nitrobenzo[d]thiazol-2-amine (2.00 g, 10.25 mmol) and 4-(2-bromoacetyl)-3-fluoro-N-methylbenzamide (2.67 g, 9.74 mmol) following a similar procedure to that described for the synthesis of methyl 4-(7-bromobenzo[d]imidazo[2,1-b]thiazol-2-yl)-3-(trifluoromethyl)benzoate, and was isolated as a dark green solid.

Yield 710 mg (20%). ¹H NMR (400 MHz, DMSO) δ 9.14 (d, J=2.0 Hz, 1H), 8.93 (d, J=3.6 Hz, 1H), 8.56 (q, J=4.4 Hz, 1H), 8.48-8.43 (m, 2H), 8.25-8.21 (m, 1H), 7.84-7.75 (m, 2H), 2.82 (d, J=4.4 Hz, 3H). m/z: [ESI⁺] 371 (M+H)⁺.

### 4-(7-aminobenzo[d]imidazo[2,1-b]thiazol-2-yl)-3-fluoro-N-methylbenzamide

A mixture of 3-fluoro-N-methyl-4-(7-nitrobenzo[d]imidazo[2,1-b]thiazol-2-yl)benzamide (500 mg, 1.350 mmol) and iron (100 mg, 1.791 mmol) in acetic acid (30 mL) was stirred for 24 h at room temperature under a nitrogen atmosphere. After filtration, the filtrate was concentrated under reduced pressure. The residue was triturated with tetrahydrofuran (10 mL) and trifluoroacetic acid (10 mL). The mixture was filtered again. The filtered cake was washed with tetrahydrofuran (5×10 mL). The combined filtrates was neutralized to pH 8 with aqueous saturated sodium carbonate. The precipitated solids were collected by filtration, washed with water (3×10 mL) and oven dried to afford 4-(7-aminobenzo[d]imidazo[2,1-b]thiazol-2-yl)-3-fluoro-N-methylbenzamide as a brown solid.

Yield 1.00 g (crude). ¹H NMR (400 MHz, CDCl₃) δ 8.32-8.28 (m, 1H), 8.14 (d, J=3.6 Hz, 1H), 7.66 (d, J=12.0 Hz, 1H), 7.57 (d, J=8.0 Hz, 1H), 7.46 (d, J=8.4 Hz, 1H), 7.00 (d, J=2.4 Hz, 1H), 6.83-6.77 (m, 1H), 6.17 (q, J=4.4 Hz, 1H), 3.06 (d, J=4.4 Hz, 3H). NH₂ protons not observed. m/z: [ESI⁺] 341 (M+H)⁺.

### tert-butyl 4-((2-(2-fluoro-4-(methylcarbamoyl)phenyl)benzo[d]imidazo[2,1-b]thiazol-7-yl)carbamoyl)piperidine-1-carboxylate

Compound tert-butyl 4-((2-(2-fluoro-4-(methylcarbamoyl)phenyl)benzo[d]imidazo[2,1-b]thiazol-7-yl)carbamoyl)piperidine-1-carboxylate was prepared from 4-(7-aminobenzo[d]imidazo[2,1-b]thiazol-2-yl)-3-fluoro-N-methylbenzamide (500 mg, 1.469 mmol) and 1-(tert-butoxycarbonyl)piperidine-4-carboxylic acid (500 mg, 2.181 mmol) following a similar procedure to that described for the synthesis of 2-bromo-N-(3-(piperidin-1-yl)propyl)benzo[d]imidazo[2,1-b]thiazole-7-carboxamide. The reaction solution was used in next step directly without any purification.

m/z: [ESI⁺] 552 (M+H)⁺.

### 4-(7-aminobenzo[d]imidazo[2,1-b]thiazol-2-yl)-N-methylbenzamide

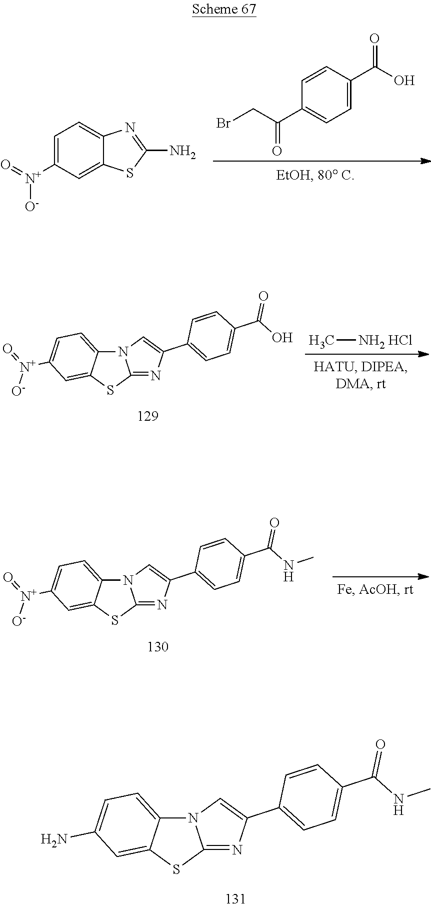

### 4-(7-nitrobenzo[d]imidazo[2,1-b]thiazol-2-yl)benzoic acid

Compound 4-(7-nitrobenzo[d]imidazo[2,1-b]thiazol-2-yl)benzoic acid was prepared from 6-nitrobenzo[d]thiazol-2-amine (5.00 g, 25.61 mmol) and 4-(2-bromoacetyl)benzoic acid (6.23 g, 25.63 mmol) following a similar procedure to that described for the synthesis of methyl 4-(7-bromobenzo[d]imidazo[2,1-b]thiazol-2-yl)-3-(trifluoromethyl)benzoate, and was isolated as a yellow solid.

Yield 2.85 g (33%). ¹H NMR (400 MHz, DMSO) δ 12.96 (br s, 1H), 9.08 (d, J=2.4 Hz, 1H), 8.99 (s, 1H), 8.44 (dd, J=2.4, 8.8 Hz, 1H), 8.15 (d, J=8.8 Hz, 1H), 8.00-7.94 (m, 4H). m/z: [ESI⁺] 340 (M+H)⁺.

### N-methyl-4-(7-nitrobenzo[d]imidazo[2,1-b]thiazol-2-yl)benzamide

Compound N-methyl-4-(7-nitrobenzo[d]imidazo[2,1-b]thiazol-2-yl)benzamide was prepared from 4-(7-nitrobenzo[d]imidazo[2,1-b]thiazol-2-yl)benzoic acid (2.85 g, 8.40 mmol) and methanamine hydrochloride (624 mg, 9.239 mmol) following a similar procedure to that described for the synthesis of 2-bromo-N-(3-(piperidin-1-yl)propyl)benzo[d]imidazo[2,1-b]thiazole-7-carboxamide, and was isolated as a yellow solid.

Yield 2.50 g (84%). ¹H NMR (400 MHz, DMSO) δ 9.15 (s, 1H), 9.05-9.01 (m, 1H), 8.51-8.48 (m, 2H), 8.27-8.14 (m, 1H), 7.97-7.91 (m, 4H), 2.86-2.75 (m, 3H). m/z: [ESI⁺] 353 (M+H)⁺.

### 4-(7-aminobenzo[d]imidazo[2,1-b]thiazol-2-yl)-N-methylbenzamide

Compound 4-(7-aminobenzo[d]imidazo[2,1-b]thiazol-2-yl)-N-methylbenzamide was prepared from N-methyl-4-(7-nitrobenzo[d]imidazo[2,1-b]thiazol-2-yl)benzamide (700 mg, 1.987 mmol) following a similar procedure to that described for the synthesis of 4-(7-aminobenzo[d]imidazo[2,1-b]thiazol-2-yl)-3-fluoro-N-methylbenzamide, and was isolated as a white solid.

Yield 250 mg (39%). ¹H NMR (400 MHz, DMSO) δ 8.68 (s, 1H), 8.43 (q, J=4.4 Hz, 1H), 7.95-7.85 (m, 4H), 7.63 (d, J=8.4 Hz, 1H), 7.06 (d, J=2.4 Hz, 1H), 6.76 (dd, J=2.4, 8.4 Hz, 1H), 5.46 (br s, 2H), 2.80 (d, J=4.4 Hz, 3H). m/z: [ESI⁺] 323 (M+H)⁺.

### tert-butyl (R)-2-(3-fluoro-4-(6-((3-(piperidin-1-yl)propyl)carbamoyl)benzo[4,5]thiazolo[3,2-b][1,2,4]triazol-2-yl)phenyl and tert-butyl (S)-2-(3-fluoro-4-(6-((3-(piperidin-1-yl)propyl)carbamoyl)benzo[4,5]thiazolo[3,2-b][1,2,4]triazol-2-yl)phenyl)pyrrolidine-1-carboxylate

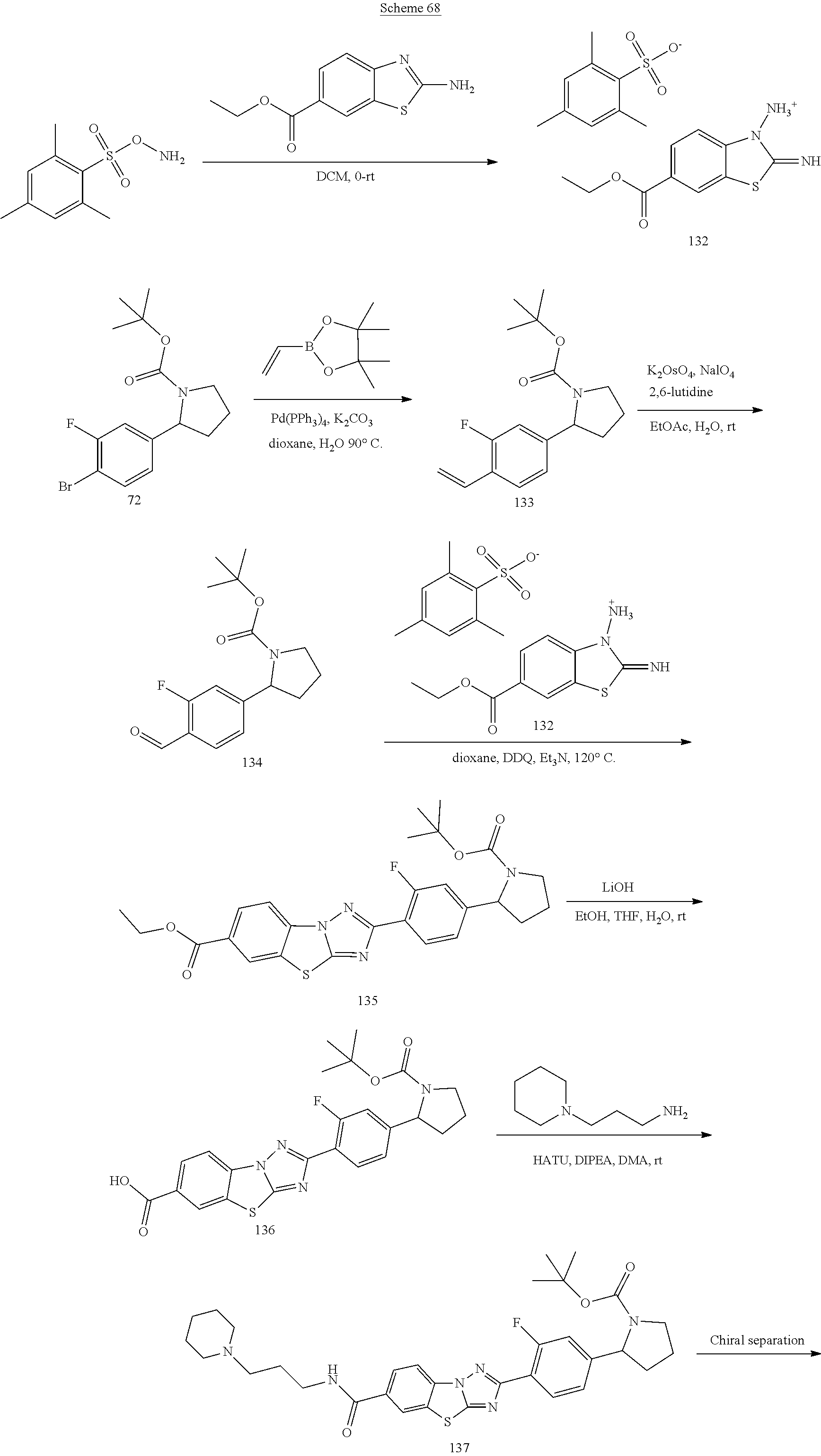

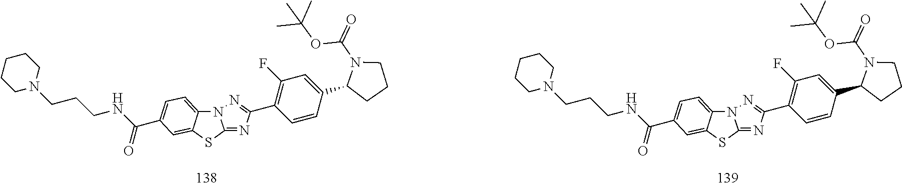 [SMILES: CC(C)(C)OC(=O)N1CCC[C@@H]1c1ccc(-c2nc3sc4cc(C(=O)NCCCN5CCCCC5)ccc4n3n2)c(F)c1 ; CC(C)(C)OC(=O)N1CCC[C@H]1c1ccc(-c2nc3sc4cc(C(=O)NCCCN5CCCCC5)ccc4n3n2)c(F)c1]

### 6-(ethoxycarbonyl)-2-iminobenzo[d]thiazol-3(2H)-aminium 2,4,6-trimethylbenzenesulfonate

To a stirred solution of ethyl 2-aminobenzo[d]thiazole-6-carboxylate (9.10 g, 40.94 mmol) in dichloromethane (200 mL) was added O-(mesitylsulfonyl)hydroxylamine (13.65 g, 63.41 mmol) portion-wise at 0° C. The reaction mixture was stirred for 16 h at room temperature under a nitrogen atmosphere. The resulting mixture was filtered. The filter cake was washed with dichloromethane (6×50 mL) and dried in a vacuum oven to afford 6-(ethoxycarbonyl)-2-iminobenzo[d]thiazol-3(2H)-aminium 2,4,6-trimethylbenzenesulfonate as a white solid.

Yield 14.65 g (82%). ¹H NMR (400 MHz, DMSO) δ 10.19 (br s, 2H, NH₂+), 8.64 (d, J=1.6 Hz, 1H), 8.16 (dd, J=1.6, 8.4 Hz, 1H), 7.66 (d, J=8.4 Hz, 1H), 6.73 (s, 2H), 6.33 (br s, 2H, NH₂+), 4.36 (q, J=7.2 Hz, 2H), 3.18 (s, 6H), 2.17 (s, 3H), 1.35 (t, J=7.2 Hz, 3H). m/z: [ESI⁺] 238 (M+H)⁺.

### tert-butyl 2-(3-fluoro-4-vinylphenyl)pyrrolidine-1-carboxylate

Compound tert-butyl 2-(3-fluoro-4-vinylphenyl)pyrrolidine-1-carboxylate was prepared from tert-butyl 2-(4-bromo-3-fluorophenyl)pyrrolidine-1-carboxylate (11.00 g, 31.96 mmol) and 4,4,5,5-tetramethyl-2-vinyl-1,3,2-dioxaborolane (24.61 g, 159.78 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a light yellow oil.

Yield 5.00 g (54%). ¹H NMR (400 MHz, CDCl₃) δ 7.76-7.54 (m, 1H), 7.52-7.40 (m, 1H), 7.00-6.92 (m, 1H), 6.91-6.80 (m, 1H), 5.88-5.73 (m, 1H), 5.41-5.28 (m, 1H), 4.98-4.68 (m, 1H), 3.70-3.46 (m, 2H), 2.42-2.20 (m, 1H), 1.98-1.74 (m, 3H), 1.67-1.06 (m, 9H). m/z: [ESI⁺] 292 (M+H)⁺.

### tert-butyl 2-(3-fluoro-4-formylphenyl)pyrrolidine-1-carboxylate

To a stirred mixture of tert-butyl 2-(3-fluoro-4-vinylphenyl)pyrrolidine-1-carboxylate (5.00 g, 17.16 mmol), sodium periodate (14.68 g, 68.64 mmol) and 2,6-lutidine (3.68 g, 34.32 mmol) in acetonitrile (25 mL) and water (25 mL) was added potassium osmate(VI) dihydrate (0.32 g, 0.86 mmol) in portion-wise at 0° C. The reaction was stirred for 2 h at room temperature under a nitrogen atmosphere. The resulting mixture was then filtered. The filter cake was washed with ethyl acetate (3×10 mL). The combined filtrate was concentrated under reduced pressure. The residue was purified by silica gel column chromatography, eluting with 1%-70% ethyl acetate in petroleum ether to afford tert-butyl 2-(3-fluoro-4-formylphenyl)pyrrolidine-1-carboxylate as a light yellow oil.

Yield 3.20 g (64%). ¹H NMR (400 MHz, CDCl₃) δ 10.34 (s, 1H), 7.90-7.80 (m, 1H), 7.15-7.05 (m, 1H), 7.01 (d, J=11.2 Hz, 1H), 5.02-4.75 (m, 1H), 3.74-3.50 (m, 2H), 2.47-2.28 (m, 1H), 1.95-1.78 (m, 3H), 1.55-1.12 (m, 9H). m/z: [ESI⁺] 294 (M+H)⁺. ethyl 2-(4-(1-(tert-butoxycarbonyl)pyrrolidin-2-yl)-2-fluorophenyl)benzo[4,5]thiazolo[3,2-b][1,2,4]triazole-6-carboxylate

To a stirred mixture of tert-butyl 2-(3-fluoro-4-formylphenyl)pyrrolidine-1-carboxylate (3.00 g, 10.23 mmol) and triethylamine (3.10 g, 30.68 mmol) in N,N-dimethylformamide (30 mL) was added ethyl 3-amino-2-imino-1,3-benzothiazole-6-carboxylate (2.43 g, 10.24 mmol) at room temperature. The resulting mixture was stirred for 1 h at 120° C. under a nitrogen atmosphere. The mixture was cooled to room temperature, and 4,5-dichloro-3,6-dioxocyclohexa-1,4-diene-1,2-dicarbonitrile (4.64 g, 20.45 mmol) was added. The resulting mixture was stirred for 16 h at 120° C. The mixture was cooled to room temperature and was purified by reverse phase flash chromatography with the following conditions: Column, Spherical C18, 20-40 μm, 330; Mobile Phase A: water (plus 10 mM formic acid); Mobile Phase B; acetonitrile; How rate: 80 mL/min; Gradient:45%-65% B in 20 min; Detector: UV 254/220 nm. The fractions containing desired product were collected and concentrated under reduced pressure to afford ethyl 2-(4-(1-(tert-butoxycarbonyl)pyrrolidin-2-yl)-2-fluorophenyl)benzo[4,5]thiazolo[3,2-b][1,2,4]triazole-6-carboxylate as a brown solid.

Yield 0.90 g (17%). ¹H NMR (400 MHz, DMSO) δ 8.90 (d, J=1.6 Hz, 1H), 8.22 (dd, J=1.6, 8.4 Hz, 1H), 8.18-8.07 (m, 2H), 7.27-7.17 (m, 2H), 4.95-4.72 (m, 1H), 4.39 (q, J=7.2 Hz, 2H), 3.63-3.44 (m, 2H), 1.93-1.71 (m, 4H), 1.49-1.04 (m, 12H). m/z: [ESI⁺] 511 (M+H)⁺.

### 2-(4-(1-(tert-butoxycarbonyl)pyrrolidin-2-yl)-2-fluorophenyl)benzo[4,5]thiazolo[3,2-b][1,2,4]triazole-6-carboxylic acid

Compound 2-(4-(1-(tert-butoxycarbonyl)pyrrolidin-2-yl)-2-fluorophenyl)benzo[4,5]thiazolo[3,2-b][1,2,4]triazole-6-carboxylic acid was prepared from ethyl 2-(4-(1-(tert-butoxycarbonyl)pyrrolidin-2-yl)-2-fluorophenyl)benzo[4,5]thiazolo[3,2-b][1,2,4]triazole-6-carboxylate (900 mg, 1.763 mmol) following a similar procedure to that described for the synthesis of 4-(7-bromobenzo[d]imidazo[2,1-b]thiazol-2-yl)-3-(trifluoromethyl)benzoic acid, and was isolated as a brown solid.

Yield 800 mg (94%). ¹H NMR (400 MHz, DMSO) δ 7 13.29 (br s, 1H), 8.86 (d, J=1.6 Hz, 1H), 8.21 (dd, J=1.6, 8.4 Hz, 1H), 8.16-8.08 (m, 2H), 7.22 (d, J=9.2 Hz, 2H), 4.95-4.72 (m, 1H), 3.67-3.50 (m, 2H), 2.44-2.24 (m, 1H), 1.96-1.70 (m, 3H), 1.47-1.05 (m, 9H). m/z: [ESI⁺] 483 (M+H)⁺.

### tert-butyl 2-(3-fluoro-4-(6-((3-(piperidin-1-yl)propyl)carbamoyl)benzo[4,5]thiazolo[3,2-b][1,2,4]triazol-2-yl)phenyl)pyrrolidine-1-carboxylate

Compound tert-butyl 2-(3-fluoro-4-(6-((3-(piperidin-1-yl)propyl)carbamoyl)benzo[4,5]thiazolo[3,2-b][1,2,4]triazol-2-yl)phenyl)pyrrolidine-1-carboxylate was prepared from 2-(4-(1-(tert-butoxycarbonyl)pyrrolidin-2-yl)-2-fluorophenyl)benzo[4,5]thiazolo[3,2-b][1,2,4]triazole-6-carboxylic acid (800 mg, 1.658 mmol) and 3-(piperidin-1-yl)propan-1-amine (354 mg, 2.489 mmol) following a similar procedure to that described for the synthesis of 2-bromo-N-(3-(piperidin-1-yl)propyl)benzo[d]imidazo[2,1-b]thiazole-7-carboxamide, and was isolated as a brown solid.

Yield 750 mg (75%). ¹H NMR (400 MHz, CD₃OD) δ 8.56-8.53 (m, 1H), 8.51 (s, 1H), 8.22-8.10 (m, 3H), 7.22 (d, J=8.0 Hz, 1H), 7.19-7.11 (m, 1H), 5.01-4.88 (m, 1H), 3.73-3.60 (m, 2H), 3.56 (t, J=6.4 Hz, 2H), 3.30-3.15 (m, 4H), 2.97-2.88 (m, 1H), 2.51-2.39 (m, 1H), 2.16-2.06 (m, 2H), 2.06-1.94 (m, 1H), 1.94-1.86 (m, 7H), 1.85-1.78 (m, 1H), 1.77-1.61 (m, 1H), 1.25 (s, 9H). m/z: [ESI⁺] 607 (M+H)⁺.

### tert-butyl (R)-2-(3-fluoro-4-(6-((3-(piperidin-1-yl)propyl)carbamoyl)benzo[4,5]thiazolo[3,2-b][1,2,4]triazol-2-yl)phenyl)pyrrolidine-1-carboxylate and tert-butyl (S)-2-(3-fluoro-4-(6-((3-(piperidin-1-yl)propyl)carbamoyl)benzo[4,5]thiazolo[3,2-b][1,2,4]triazol-2-yl)phenyl)pyrrolidine-1-carboxylate

Tert-butyl 2-(3-fluoro-4-(6-((3-(piperidin-1-yl)propyl)carbamoyl)benzo[4,5]thiazolo[3,2-b][1,2,4]triazol-2-yl)phenyl)pyrrolidine-1-carboxylate (500 mg, 0.824 mmol) was separated by chiral HPLC with the following conditions: Column: CHIRALPAK IF, 2×25 cm, 5 m; Mobile Phase A: Hexane (0.5% 2 M NH₃-MeOH, v/v), Mobile Phase B: MeOH:EtOH=1:1, v/v; Flow rate: 15 mL/min; Gradient: 40% B in 32 min; Detector: UV 254/220 nm; RT1(min): 19.42; RT2(min): 25.72). The fractions containing the faster eluting peak were concentrated under reduced pressure to afford tert-butyl (R)-2-(3-fluoro-4-(6-((3-(piperidin-1-yl)propyl)carbamoyl)benzo[4,5]thiazolo[3,2-b][1,2,4]triazol-2-yl)phenyl)pyrrolidine-1-carboxylate as a brown solid.

Yield 232 mg (46%). ¹H NMR (400 MHz, CDCl₃) δ 9.16 (s, 1H), 8.46 (s, 1H), 8.17 (t, J=7.6 Hz, 1H), 8.13-8.06 (m, 2H), 7.16-7.06 (m, 2H), 5.06-4.78 (m, 1H), 3.72-3.57 (m, 4H), 2.76-2.33 (m, 6H), 1.98-1.84 (m, 4H), 1.62-1.45 (m, 8H), 1.27 (s, 9H). m/z: [ESI⁺] 607 (M+H)⁺.

The fractions containing the slower eluting peak were concentrated under reduced pressure to afford tert-butyl (S)-2-(3-fluoro-4-(6-((3-(piperidin-1-yl)propyl)carbamoyl)benzo[4,5]thiazolo[3,2-b][1,2,4]triazol-2-yl)phenyl)pyrrolidine-1-carboxylate as a brown solid.

Yield 230 mg (46%). ¹H NMR (400 MHz, CDCl₃) δ 8.93 (s, 1H), 8.45 (s, 1H), 8.16 (t, J=7.6 Hz, 1H), 8.14-8.07 (m, 2H), 7.16-7.05 (m, 2H), 5.08-4.77 (m, 1H), 3.79-3.58 (m, 4H), 2.92-2.53 (m, 6H), 2.47-2.33 (m, 1H), 2.05-1.84 (m, 5H), 1.70-1.36 (m, 6H), 1.27 (s, 9H). m/z: [ESI⁺] 607 (M+H)⁺.

### 2-(2-fluoro-4-(methylcarbamoyl)phenyl)benzo[4,5]thiazolo[3,2-b][1,2,4]triazole-6-carboxylic acid

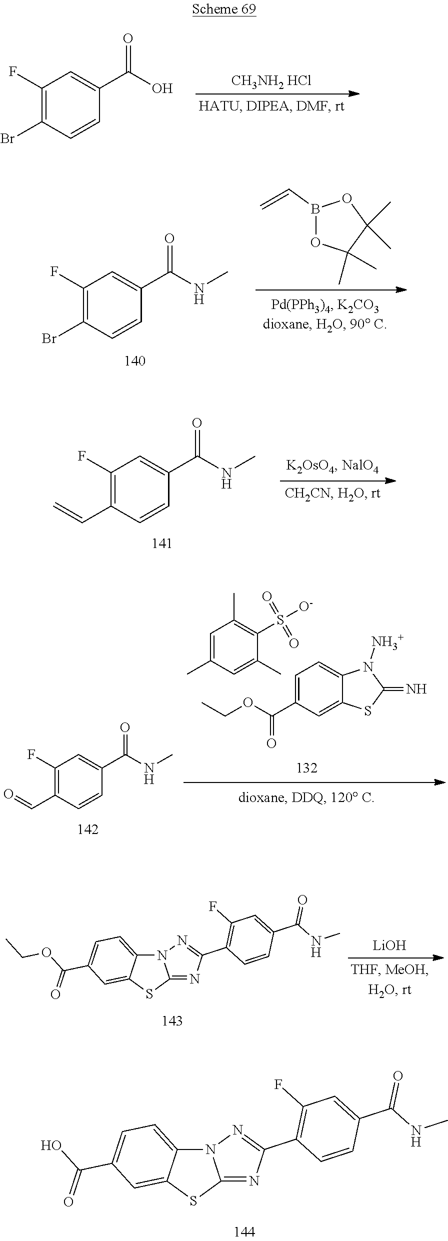

### 4-bromo-3-fluoro-N-methylbenzamide

Compound 4-bromo-3-fluoro-N-methylbenzamide was prepared from 4-bromo-3-fluorobenzoic acid (10.00 g, 45.66 mmol) and methanamine hydrochloride (4.01 g, 59.39 mmol) following a similar procedure to that described for the synthesis of 2-bromo-N-(3-(piperidin-1-yl)propyl)benzo[d]imidazo[2,1-b]thiazole-7-carboxamide, and was isolated as a white solid.

Yield 10.00 g (94%). ¹H NMR (400 MHz, CDCl₃) δ 7.70-7.54 (m, 2H), 7.43 (dd, J=2.0, 8.4 Hz, 1H), 6.59 (br s, 1H), 3.01 (d, J=4.8 Hz, 3H). m/z: [ESI⁺] 232, 234 (M+H)⁺.

### 3-fluoro-N-methyl-4-vinylbenzamide

Compound 3-fluoro-N-methyl-4-vinylbenzamide was prepared from 4-bromo-3-fluoro-N-methylbenzamide (1.00 g, 4.31 mmol) and 4,4,5,5-tetramethyl-2-vinyl-1,3,2-dioxaborolane (3.32 g, 21.56 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a light brown solid.

Yield 0.70 g (91%). ¹H NMR (400 MHz, CDCl₃) δ 7.59-7.44 (m, 3H), 6.89 (dd, J=11.2, 17.6 Hz, 1H), 6.24 (br, s, 1H), 5.92 (dd, J=1.2, 17.6 Hz, 1H), 5.49 (dd, J=1.2, 11.2 Hz, 1H), 3.03 (d, J=4.8 Hz, 3H). m/z: [ESI⁺] 180 (M+H)⁺.

### 3-fluoro-4-formyl-N-methylbenzamide

Compound 3-fluoro-4-formyl-N-methylbenzamide was prepared from 3-fluoro-N-methyl-4-vinylbenzamide (2.60 g, 14.51 mmol) following a similar procedure to that described for the synthesis of tert-butyl 2-(3-fluoro-4-formylphenyl)pyrrolidine-1-carboxylate, and was isolated as an off-white solid.

Yield 1.80 g (68%). ¹H NMR (400 MHz, DMSO) δ 10.42 (s, 1H), 7.96 (dd, J=6.8, 8.0 Hz, 1H), 7.72-7.56 (m, 2H), 6.20 (br, s, 1H), 3.07 (d, J=4.8 Hz, 3H). m/z: [ESI⁺] 182 (M+H)⁺.

### ethyl 2-(2-fluoro-4-(methylcarbamoyl)phenyl)benzo[4,5]thiazolo[3,2-b][1,2,4]triazole-6-carboxylate

Compound ethyl 2-(2-fluoro-4-(methylcarbamoyl)phenyl)benzo[4,5]thiazolo[3,2-b][1,2,4]triazole-6-carboxylate was prepared from 3-fluoro-4-formyl-N-methylbenzamide (600 mg, 3.312 mmol) and 6-(ethoxycarbonyl)-2-iminobenzo[d]thiazol-3(2H)-aminium 2,4,6-trimethylbenzenesulfonate (786 mg, 1.796 mmol) following a similar procedure to that described for the synthesis of ethyl 2-(4-(1-(tert-butoxycarbonyl)pyrrolidin-2-yl)-2-fluorophenyl)benzo[4,5]thiazolo[3,2-b][1,2,4]triazole-6-carboxylate, and was isolated as a brown solid.

Yield 240 mg (34%). ¹H NMR (400 MHz, DMSO) δ 8.90 (s, 1H), 8.67 (d, J=5.2 Hz, 1H), 8.31-8.09 (m, 3H), 7.88-7.77 (m, 2H), 4.38 (q, J=7.2 Hz, 2H), 2.82 (d, J=4.4 Hz, 3H), 1.38 (t, J=7.2 Hz, 3H). m/z: [ESI⁺] 399 (M+H)⁺.

### 2-(2-fluoro-4-(methylcarbamoyl)phenyl)benzo[4,5]thiazolo[3,2-b][1,2,4]triazole-6-carboxylic acid

Compound 2-(2-fluoro-4-(methylcarbamoyl)phenyl)benzo[4,5]thiazolo[3,2-b][1,2,4]triazole-6-carboxylic acid was prepared from ethyl 2-(2-fluoro-4-(methylcarbamoyl)phenyl)benzo[4,5]thiazolo[3,2-b][1,2,4]triazole-6-carboxylate (870 mg, 2.184 mmol) following a similar procedure to that described for the synthesis of 4-(7-bromobenzo[d]imidazo[2,1-b]thiazol-2-yl)-3-(trifluoromethyl)benzoic acid, and was isolated as a brown solid.

Yield 660 mg (82%). ¹H NMR (400 MHz, DMSO) δ 13.27 (br, s, 1H), 8.86 (d, J=1.6 Hz, 1H), 8.67-8.61 (m, 1H), 8.29-8.18 (m, 2H), 8.17-8.12 (m, 1H), 7.89-7.78 (m, 2H), 2.83 (d, J=4.4 Hz, 3H). m/z: [ESI⁺]371 (M+H)⁺.

### tert-butyl (3-fluoro-4-(6-((3-(piperidin-1-yl)propyl)carbamoyl)benzo[4,5]thiazolo[3,2-b][1,2,4]triazol-2-yl)benzyl)carbamate

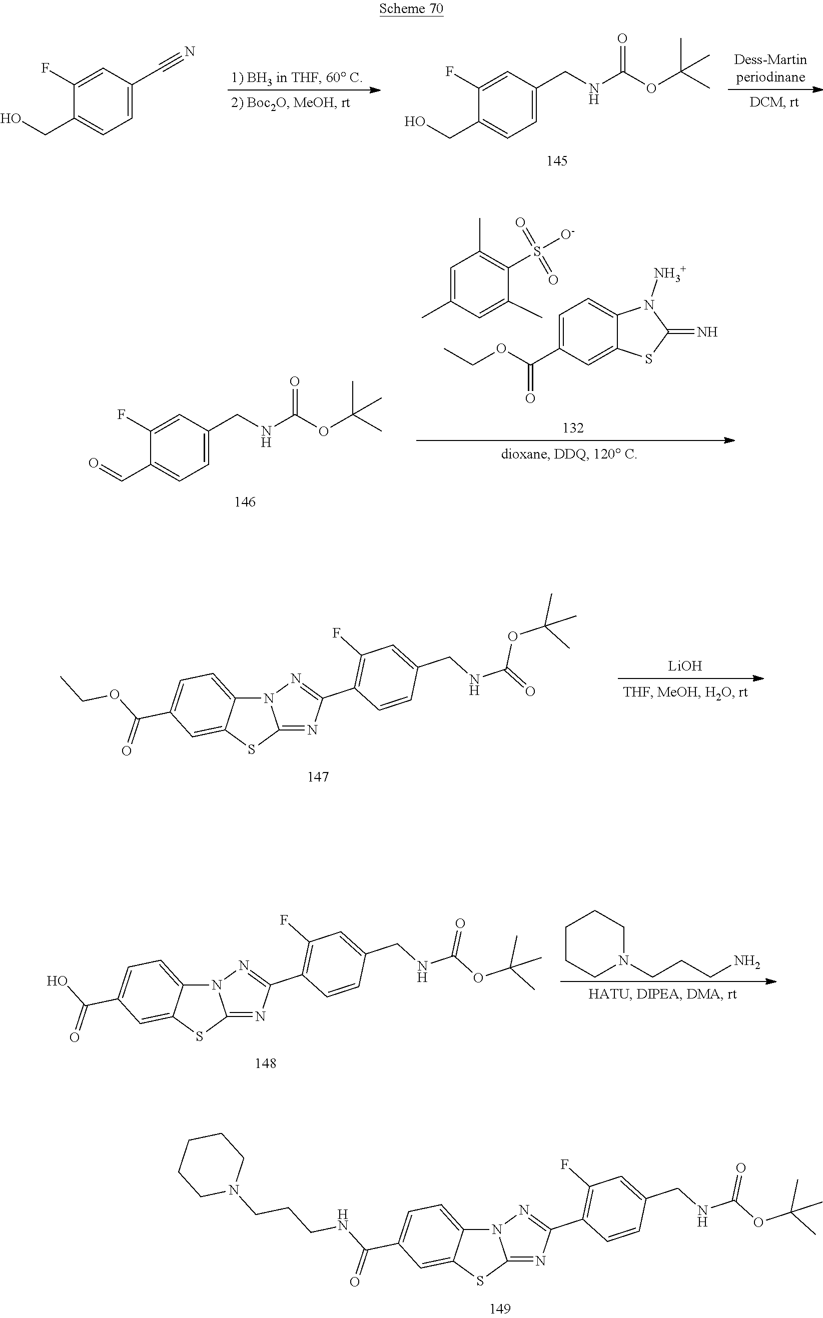

### tert-butyl (3-fluoro-4-(hydroxymethyl)benzyl)carbamate

Compound tert-butyl (3-fluoro-4-(hydroxymethyl)benzyl)carbamate was prepared from 3-fluoro-4-(hydroxymethyl)benzonitrile (1.00 g, 6.62 mmol) following a similar procedure to that described for the synthesis of tert-butyl (4-bromo-3-cyclopropylbenzyl)carbamate, and was isolated as a light yellow oil.

Yield 1.20 g (71%). ¹H NMR (400 MHz, DMSO) δ 7.45-7.35 (m, 2H), 7.05 (dd, J=1.6, 7.6 Hz, 1H), 6.98 (dd, J=1.6, 11.2 Hz, 1H), 5.20 (t, J=5.6 Hz, 1H), 4.51 (d, J=4.8 Hz, 2H), 4.11 (d, J=6.0 Hz, 2H), 1.40 (s, 9H). m/z: [ESI⁺] 278 (M+Na)⁺.

### tert-butyl (3-fluoro-4-formylbenzyl)carbamate

To a stirred solution of tert-butyl (3-fluoro-4-(hydroxymethyl)benzyl)carbamate (4.00 g, 15.67 mmol) in dichloromethane (40 mL) was added Dess-Martin periodinane (6.65 g, 15.68 mmol) portion-wise at 0° C. The reaction mixture was then stirred for 1 h at 0° C. After filtration, the filter cake was washed with dichloromethane (3×100 mL). The combined filtrate was concentrated under reduced pressure to afford tert-butyl (3-fluoro-4-formylbenzyl)carbamate as a light brown oil.

Yield 3.00 g (76%). ¹H NMR (400 MHz, CDCl₃) δ 10.34 (s, 1H), 8.02 (d, J=8.0 Hz, 1H), 7.19 (d, J=8.0 Hz, 1H), 7.15-7.10 (m, 1H), 5.08 (t, J=6.4 Hz, 1H), 4.39 (d, J=6.4 Hz, 2H), 1.49 (s, 9H). m/z: [ESI⁺] 198 (M+H−56)⁺.

### ethyl 2-(4-(((tert-butoxycarbonyl)amino)methyl)-2-fluorophenyl)benzo[4,5]thiazolo[3,2-b][1,2,4]triazole-6-carboxylate

Compound ethyl 2-(4-(((tert-butoxycarbonyl)amino)methyl)-2-fluorophenyl)benzo[4,5]thiazolo[3,2-b][1,2,4]triazole-6-carboxylate was prepared from tert-butyl (3-fluoro-4-formylbenzyl)carbamate (1.00 g, 3.95 mmol) and 6-(ethoxycarbonyl)-2-iminobenzo[d]thiazol-3(2H)-aminium 2,4,6-trimethylbenzenesulfonate (1.73 g, 3.95 mmol) following a similar procedure to that described for the synthesis of ethyl 2-(4-(1-(tert-butoxycarbonyl)pyrrolidin-2-yl)-2-fluorophenyl)benzo[4,5]thiazolo[3,2-b][1,2,4]triazole-6-carboxylate, and was isolated as a brown solid.

Yield 0.18 g (10%). ¹H NMR (400 MHz, DMSO) δ 8.88 (d, J=1.6 Hz, 1H), 8.25-8.17 (m, 1H), 8.16-8.07 (m, 2H), 7.53 (t, J=6.4 Hz, 1H), 7.30-7.22 (m, 2H), 4.38 (q, J=7.2 Hz, 2H), 4.23 (d, J=6.0 Hz, 2H), 1.41 (s, 9H), 1.37 (d, J=7.2 Hz, 3H). m/z: [ESI⁺] 471 (M+H)⁺.

### 2-(4-(((tert-butoxycarbonyl)amino)methyl)-2-fluorophenyl)benzo[4,5]thiazolo[3,2-b][1,2,4]triazole-6-carboxylic acid

Compound 2-(4-(((tert-butoxycarbonyl)amino)methyl)-2-fluorophenyl)benzo[4,5]thiazolo[3,2-b][1,2,4]triazole-6-carboxylic acid was prepared from ethyl 2-(4-(((tert-butoxycarbonyl)amino)methyl)-2-fluorophenyl)benzo[4,5]thiazolo[3,2-b][1,2,4]triazole-6-carboxylate (300 mg, 0.638 mmol) following a similar procedure to that described for the synthesis of 4-(7-bromobenzo[d]imidazo[2,1-b]thiazol-2-yl)-3-(trifluoromethyl)benzoic acid, and was isolated as a light yellow solid.

Yield 200 mg (71%). ¹H NMR (400 MHz, DMSO) δ 13.29 (br s, 1H), 8.86 (d, J=1.6 Hz, 1H), 8.23-8.18 (m, 1H), 8.17-8.08 (m, 2H), 7.51 (t, J=6.0 Hz, 1H), 7.30-7.21 (m, 2H), 4.23 (d, J=6.0 Hz, 2H), 1.42 (s, 9H), m/z: [ESI⁺] 443 (M+H)⁺.

### tert-butyl (3-fluoro-4-(6-((3-(piperidin-1-yl)propyl)carbamoyl)benzo[4,5]thiazolo[3,2-b][1,2,4]triazol-2-yl)benzyl)carbamate

Compound tert-butyl (3-fluoro-4-(6-((3-(piperidin-1-yl)propyl)carbamoyl)benzo[4,5]thiazolo[3,2-b][1,2,4]triazol-2-yl)benzyl)carbamate was prepared from 2-(4-(((tert-butoxycarbonyl)amino)methyl)-2-fluorophenyl)benzo[4,5]thiazolo[3,2-b][1,2,4]triazole-6-carboxylic acid (130 mg, 0.294 mmol) and 3-(piperidin-1-yl)propan-1-amine (83 mg, 0.583 mmol) following a similar procedure to that described for the synthesis of 2-bromo-N-(3-(piperidin-1-yl)propyl)benzo[d]imidazo[2,1-b]thiazole-7-carboxamide, and was isolated as a light brown solid.

Yield 100 mg (60%). ¹H NMR (400 MHz, CDCl₃) δ 8.91 (br s, 1H), 8.65 (s, 1H), 8.35 (d, J=8.4 Hz, 1H), 8.22-8.16 (m, 1H), 8.13 (d, J=8.4 Hz, 1H), 7.34-7.20 (m, 2H), 5.37 (br s, 1H), 4.44-4.39 (m, 2H), 3.76-3.69 (m, 2H), 3.64-3.57 (m, 2H), 3.14-3.10 (m, 2H), 2.68-2.58 (m, 2H), 2.32-2.20 (m, 2H), 1.75-1.56 (m, 6H), 1.49 (s, 9H). m/z: [ESI⁺] 567 (M+H)⁺.

### tert-butyl (4-(6-((3-(diethylamino)propyl)carbamoyl)benzo[4,5]thiazolo[3,2-b][1,2,4]triazol-2-yl)-3-fluorobenzyl)carbamate

Compound tert-butyl (4-(6-((3-(diethylamino)propyl)carbamoyl)benzo[4,5]thiazolo[3,2-b][1,2,4]triazol-2-yl)-3-fluorobenzyl)carbamate was prepared from 2-(4-(((tert-butoxycarbonyl)amino)methyl)-2-fluorophenyl)benzo[4,5]thiazolo[3,2-b][1,2,4]triazole-6-carboxylic acid (150 mg, 0.339 mmol) and N′,N¹-diethylpropane-1,3-diamine (66 mg, 0.507 mmol) following a similar procedure to that described for the synthesis of 2-bromo-N-(3-(piperidin-1-yl)propyl)benzo[d]imidazo[2,1-b]thiazole-7-carboxamide, and was isolated as a light brown solid.

Yield 160 mg (85%). ¹H NMR (400 MHz, DMSO) δ 8.77 (t, J=4.0 Hz, 1H), 8.68 (s, 1H), 8.18-8.05 (m, 2H), 7.75 (s, 1H), 7.52 (t, J=4.0 Hz, 1H), 7.33-7.20 (m, 2H), 4.23 (d, J=6.4 Hz, 2H), 3.35 (q, J=6.4 Hz, 2H), 3.32-3.21 (m, 3H), 3.16 (q, J=6.4 Hz, 2H), 2.78-2.70 (m, 2H), 2.48-2.38 (m, 1H), 1.42 (s, 9H), 1.02 (t, J=7.2 Hz, 6H). m/z: [ESI⁺] 555 (M+H)⁺.

### tert-butyl (3-fluoro-4-(6-((3-(4-fluoropiperidin-1-yl)propyl)carbamoyl)benzo[4,5]thiazolo[3,2-b][1,2,4]triazol-2-yl)benzyl)carbamate

Compound tert-butyl (3-fluoro-4-(6-((3-(4-fluoropiperidin-1-yl)propyl)carbamoyl)benzo[4,5]thiazolo[3,2-b][1,2,4]triazol-2-yl)benzyl)carbamate was prepared from 2-(4-(((tert-butoxycarbonyl)amino)methyl)-2-fluorophenyl)benzo[4,5]thiazolo[3,2-b][1,2,4]triazole-6-carboxylic acid (250 mg, 0.565 mmol) and 3-(4-fluoropiperidin-1-yl)propan-1-amine dihydrochloride (136 mg, 0.583 mmol) following a similar procedure to that described for the synthesis of 2-bromo-N-(3-(piperidin-1-yl)propyl)benzo[d]imidazo[2,1-b]thiazole-7-carboxamide, and was isolated as a brown solid.

Yield 200 mg (61%). ¹H NMR (400 MHz, DMSO) δ 8.81 (t, J=5.6 Hz, 1H), 8.67 (s, 1H), 8.19-8.08 (m, 3H), 7.51 (t, J=5.6 Hz, 1H), 7.30-7.22 (m, 2H), 5.05-4.81 (m, 1H), 4.23 (d, J=6.0 Hz, 2H), 3.46-3.35 (m, 2H), 3.23-3.07 (m, 4H), 2.05-1.89 (m, 6H), 1.42 (s, 9H), 1.37-1.32 (m, 1H), 1.30-1.22 (m, 1H). m/z: [ESI⁺] 585 (M+H)⁺.

### tert-butyl cyclopropyl(4-(6-((3-(piperidin-1-yl)propyl)carbamoyl)benzo[4,5]thiazolo[3,2-b][1,2,4]triazol-2-yl)benzyl)carbamate

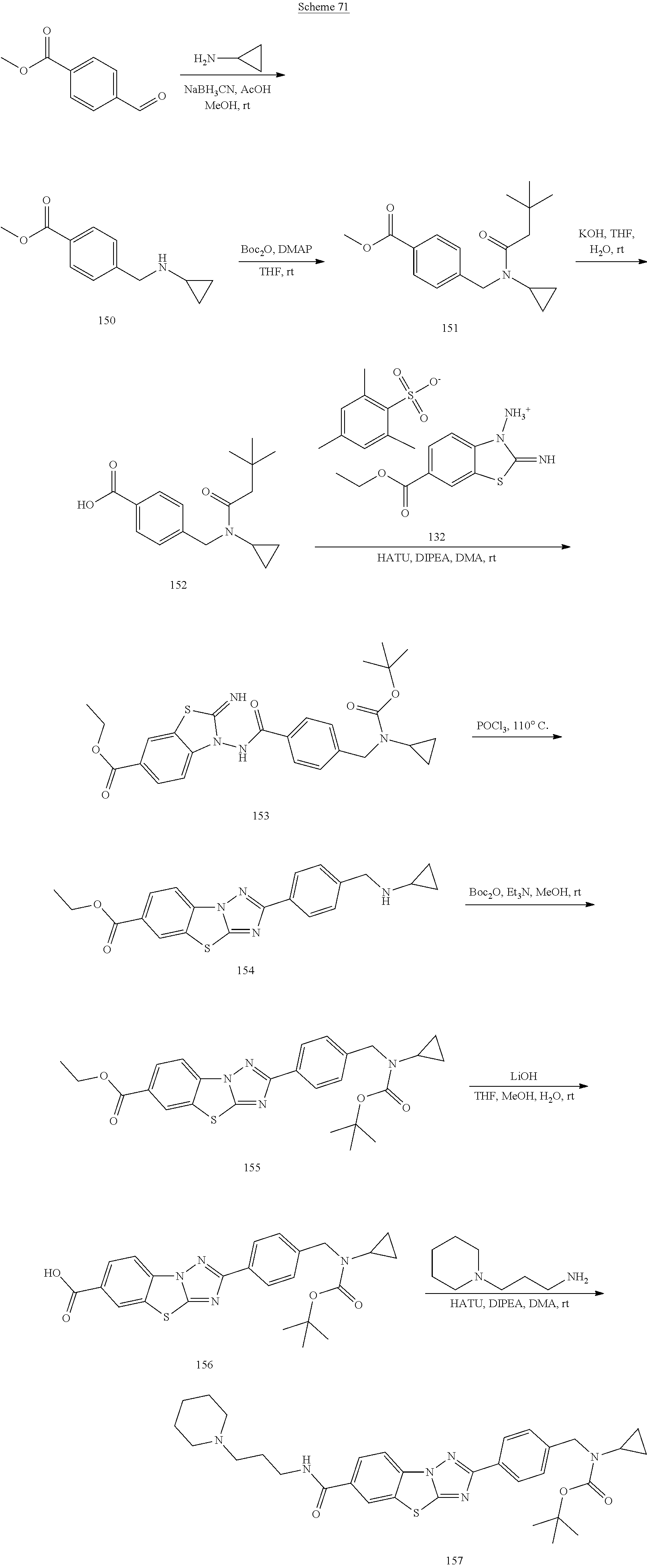

### methyl 4-((cyclopropylamino)methyl)benzoate

Compound methyl 4-((cyclopropylamino)methyl)benzoate was prepared from methyl 4-formylbenzoate (1.00 g, 6.09 mmol) and cyclopropanamine (0.38 g, 6.66 mmol) following a similar procedure to that described for the synthesis of tert-butyl (3-(4-fluoropiperidin-1-yl)propyl)carbamate, and was isolated as a yellow oil.

Yield 0.70 g (56%). ¹H NMR (400 MHz, DMSO) δ 7.99-7.81 (m, 2H), 7.52-7.40 (m, 2H), 3.84 (s, 3H), 3.79 (s, 2H), 2.82 (s, 1H), 2.21-2.13 (m, 1H), 0.41-0.30 (m, 2H), 0.28-0.17 (m, 2H). m/z: [ESI⁺] 206 (M+H)⁺.

### methyl 4-(((tert-butoxycarbonyl)(cyclopropyl)amino)methyl)benzoate

Compound methyl 4-(((tert-butoxycarbonyl)(cyclopropyl)amino)methyl)benzoate was prepared from methyl 4-((cyclopropylamino)methyl)benzoate (0.70 g, 3.41 mmol) following a similar procedure to that described for the synthesis of tert-butyl 2-(4-bromophenyl)pyrrolidine-1-carboxylate, and was isolated as a colorless oil.

Yield 0.87 g (84%). ¹H NMR (400 MHz, CDCl₃) δ 8.05-7.97 (m, 2H), 7.36-7.29 (m, 2H), 4.49 (s, 2H), 3.93 (s, 3H), 2.58-2.47 (m, 1H), 1.48 (s, 9H), 0.78-0.70 (m, 2H), 0.68-0.62 (m, 2H). m/z: [ESI⁺] 250 (M-56+H)⁺.

### 4-(((tert-butoxycarbonyl)(cyclopropyl)amino)methyl)benzoic acid

Compound 4-(((tert-butoxycarbonyl)(cyclopropyl)amino)methyl)benzoic acid was prepared from methyl 4-(((tert-butoxycarbonyl)(cyclopropyl)amino)methyl)benzoate (0.87 g, 2.85 mmol) following a similar procedure to that described for the synthesis of 4-(7-bromobenzo[d]imidazo[2,1-b]thiazol-2-yl)-3-(trifluoromethyl)benzoic acid, and was isolated as a white solid.

Yield 0.60 g (72%). ¹H NMR (400 MHz, DMSO) δ 12.86 (s, 1H), 7.95-7.88 (m, 2H), 7.35-7.28 (m, 2H), 4.43 (s, 2H), 2.49-2.46 (m, 1H), 1.39 (s, 9H), 0.72-0.63 (m, 2H), 0.63-0.56 (m, 2H). m/z: [ESI] 290 (M−H)⁻.

### ethyl 3-(4-(((tert-butoxycarbonyl)(cyclopropyl)amino)methyl)benzamido)-2-imino-2,3-dihydrobenzo[d]thiazole-6-carboxylate

Compound ethyl 3-(4-(((tert-butoxycarbonyl)(cyclopropyl)amino)methyl)benzamido)-2-imino-2,3-dihydrobenzo[d]thiazole-6-carboxylate was prepared from 4-(((tert-butoxycarbonyl)(cyclopropyl)amino)methyl)benzoic acid (4.00 g, 13.73 mmol) and 6-(ethoxycarbonyl)-2-iminobenzo[d]thiazol-3(2H)-aminium 2,4,6-trimethylbenzenesulfonate (4.60 g, 10.51 mmol) following a similar procedure to that described for the synthesis of 2-bromo-N-(3-(piperidin-1-yl)propyl)benzo[d]imidazo[2,1-b]thiazole-7-carboxamide, and was isolated as a light brown solid.

Yield 5.21 g (97%). ¹H NMR (400 MHz, DMSO) δ 8.54 (d, J=1.6 Hz, 1H), 8.36 (d, J=8.0 Hz, 2H), 8.13 (dd, J=1.6, 8.4 Hz, 1H), 7.76 (d, J=8.4 Hz, 1H), 7.35 (d, J=8.0 Hz, 2H), 6.43 (br s, 2H), 4.46 (s, 2H), 4.36 (q, J=7.2 Hz, 2H), 2.50-2.45 (m, 1H), 1.41 (s, 9H), 1.36 (t, J=7.2 Hz, 3H), 0.72-0.65 (m, 2H), 0.65-0.57 (m, 2H). m/z: [ESI⁺] 511 (M+H)⁺.

### ethyl 2-(4-((cyclopropylamino)methyl)phenyl)benzo[4,5]thiazolo[3,2-b][1,2,4]triazole-6-carboxylate

A mixture of ethyl 3-(4-(((tert-butoxycarbonyl)(cyclopropyl)amino)methyl)benzamido)-2-imino-2,3-dihydrobenzo[d]thiazole-6-carboxylate (4.00 g, 7.83 mmol) in phosphorus(V) oxychloride (40 mL) was stirred for 2 h at 110° C. under a nitrogen atmosphere. The resulting mixture was allowed to cool to room temperature and was concentrated under reduced pressure. The residue was diluted with ice/water (50 mL) and the resulting solids were filtered. The filter cake was washed with water (3×100 mL) and oven dried to afford ethyl 2-(4-((cyclopropylamino)methyl)phenyl)benzo[4,5]thiazolo[3,2-b][1,2,4]triazole-6-carboxylate as a brown solid.

Yield 2.54 g (83%). ¹H NMR (400 MHz, CD₃OD) δ 8.65 (s, 1H), 8.30-8.21 (m, 3H), 8.05 (d, J=8.4 Hz, 1H), 7.67 (d, J=8.0 Hz, 2H), 4.44 (q, J=7.2 Hz, 2H), 4.40 (s, 2H), 2.89-2.76 (m, 1H), 1.45 (t, J=7.2 Hz, 3H), 1.01-0.91 (m, 4H). NH proton not observed. m/z: [ESI⁺] 393 (M+H)⁺.

### ethyl 2-(4-(((tert-butoxycarbonyl)(cyclopropyl)amino)methyl)phenyl)benzo[4,5]thiazolo[3,2-b][1,2,4]triazole-6-carboxylate

Compound ethyl 2-(4-(((tert-butoxycarbonyl)(cyclopropyl)amino)methyl)phenyl)benzo[4,5]thiazolo[3,2-b][1,2,4]triazole-6-carboxylate was prepared from ethyl 2-(4-((cyclopropylamino)methyl)phenyl)benzo[4,5]thiazolo[3,2-b][1,2,4]triazole-6-carboxylate (2.50 g, 6.37 mmol) following a similar procedure to that described for the synthesis of tert-butyl 2-(4-bromophenyl)pyrrolidine-1-carboxylate, and was isolated as a dark brown solid.

Yield 1.98 g (63%). ¹H NMR (400 MHz, DMSO) δ 8.89 (d, J=1.6 Hz, 1H), 8.25-8.20 (m, 1H), 8.19-8.11 (m, 3H), 7.39 (d, J=8.0 Hz, 2H), 4.45 (s, 2H), 4.39 (q, J=7.2 Hz, 2H), 2.55-2.52 (m, 1H), 1.41 (s, 9H), 1.37 (t, J=7.2 Hz, 3H), 0.74-0.66 (m, 2H), 0.66-0.59 (m, 2H). m/z: [ESI⁺] 493 (M+H)⁺.

### 2-(4-(((tert-butoxycarbonyl)(cyclopropyl)amino)methyl)phenyl)benzo[4,5]thiazolo[3,2-b][1,2,4]triazole-6-carboxylic acid

Compound 2-(4-(((tert-butoxycarbonyl)(cyclopropyl)amino)methyl)phenyl)benzo[4,5]thiazolo[3,2-b][1,2,4]triazole-6-carboxylic acid was prepared from ethyl 2-(4-(((tert-butoxycarbonyl)(cyclopropyl)amino)methyl)phenyl)benzo[4,5]thiazolo[3,2-b][1,2,4]triazole-6-carboxylate (1.98 g, 4.02 mmol) following a similar procedure to that described for the synthesis of 4-(7-bromobenzo[d]imidazo[2,1-b]thiazol-2-yl)-3-(trifluoromethyl)benzoic acid, and was isolated as a brown solid.

Yield 1.50 g (80%). ¹H NMR (400 MHz, DMSO) δ 13.30 (s, 1H), 8.84 (d, J=1.6 Hz, 1H), 8.20 (dd, J=1.6, 8.4 Hz, 1H), 8.18-8.11 (m, 3H), 7.39 (d, J=8.0 Hz, 2H), 4.44 (s, 2H), 2.51-2.50 (m, 1H), 1.42 (s, 9H), 0.73-0.66 (m, 2H), 0.66-0.59 (m, 2H). m/z: [ESI⁺] 465 (M+H)⁺.

### tert-butyl cyclopropyl(4-(6-((3-(piperidin-1-yl)propyl)carbamoyl)benzo[4,5]thiazolo[3,2-b][1,2,4]triazol-2-yl)benzyl)carbamate

Compound tert-butyl cyclopropyl(4-(6-((3-(piperidin-1-yl)propyl)carbamoyl)benzo[4,5]thiazolo[3,2-b][1,2,4]triazol-2-yl)benzyl)carbamate was prepared from 2-(4-(((tert-butoxycarbonyl)(cyclopropyl)amino)methyl)phenyl)benzo[4,5]thiazolo[3,2-b][1,2,4]triazole-6-carboxylic acid (500 mg, 1.076 mmol) and 3-(piperidin-1-yl)propan-1-amine (230 mg, 1.617 mmol) following a similar procedure to that described for the synthesis of 2-bromo-N-(3-(piperidin-1-yl)propyl)benzo[d]imidazo[2,1-b]thiazole-7-carboxamide, and was isolated as an off-white solid.

Yield 527 mg (83%). ¹H NMR (400 MHz, DMSO) δ 8.81 (t, J=5.6 Hz, 1H), 8.67 (s, 1H), 8.19-8.10 (m, 4H), 7.40 (d, J=8.0 Hz, 2H), 4.45 (s, 2H), 3.43-3.36 (m, 1H), 3.19-3.10 (m, 1H), 3.02-2.98 (m, 2H), 2.54-2.52 (m, 1H), 1.94-1.90 (m, 1H), 1.83-1.68 (m, 3H), 1.41 (s, 9H), 1.32-1.21 (m, 4H), 0.91-0.81 (m, 4H), 0.74-0.67 (m, 2H), 0.67-0.60 (m, 2H). m/z: [ESI⁺] 589 (M+H)⁺.

### tert-butyl cyclopropyl(4-(6-((3-(diethylamino)propyl)carbamoyl)benzo[4,5]thiazolo[3,2-b][1,2,4]triazol-2-yl)benzyl)carbamate

Compound tert-butyl cyclopropyl(4-(6-((3-(diethylamino)propyl)carbamoyl)benzo[4,5]thiazolo[3,2-b][1,2,4]triazol-2-yl)benzyl)carbamate was prepared from 2-(4-(((tert-butoxycarbonyl)(cyclopropyl)amino)methyl)phenyl)benzo[4,5]thiazolo[3,2-b][1,2,4]triazole-6-carboxylic acid (500 mg, 1.076 mmol) and N¹,N¹-diethylpropane-1,3-diamine (210 mg, 1.612 mmol) following a similar procedure to that described for the synthesis of 2-bromo-N-(3-(piperidin-1-yl)propyl)benzo[d]imidazo[2,1-b]thiazole-7-carboxamide, and was isolated as an off-white solid.

Yield 308 mg (50%). ¹H NMR (300 MHz, DMSO) δ 8.79 (t, J=5.6 Hz, 1H), 8.67 (s, 1H), 8.17-8.11 (m, 4H), 7.40 (d, J=8.0 Hz, 2H), 4.45 (s, 2H), 3.39 (q, J=6.4 Hz, 2H), 3.02-2.95 (m, 2H), 2.54-2.52 (m, 1H), 1.92-1.79 (m, 2H), 1.41 (s, 9H), 1.32-1.19 (m, 4H), 1.13 (t, J=7.2 Hz, 6H), 0.73-0.67 (m, 2H), 0.66-0.60 (m, 2H). m/z: [ESI⁺] 577 (M+H)⁺.

### tert-butyl cyclopropyl(4-(6-((3-(diethylamino)propyl)carbamoyl)benzo[4,5]thiazolo[3,2-b][1,2,4]triazol-2-yl)-3-fluorobenzyl)carbamate

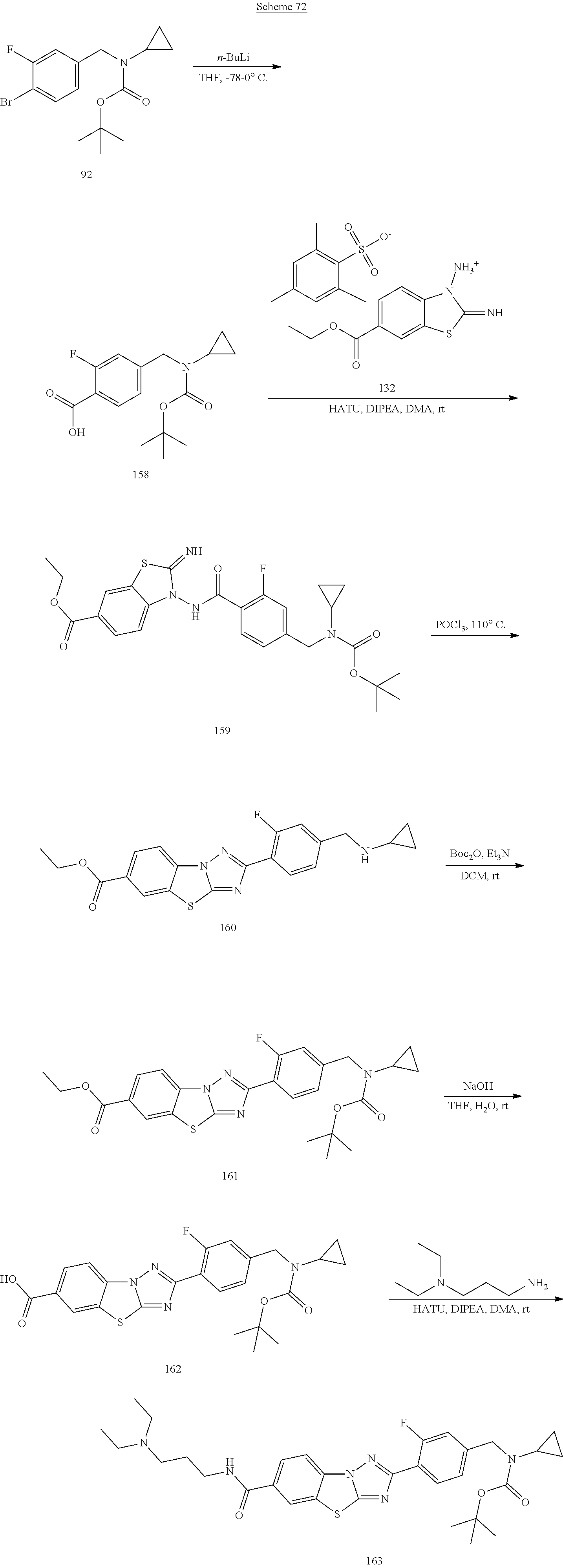

### 4-(((tert-butoxycarbonyl)(cyclopropyl)amino)methyl)-2-fluorobenzoic acid

n-Butyl lithium (2.5 M in THF, 29 mL, 72.50 mmol) was added drop-wise to a solution of tert-butyl (4-bromo-3-fluorobenzyl)(cyclopropyl)carbamate (10.00 g, 29.05 mmol) in tetrahydrofuran (250 mL) at −78° C. under a nitrogen atmosphere. The reaction solution was stirred for additional 1 h at −78° C. To resulting mixture was added dry ice (30 g) at −78° C. and was stirred for additional 1 h at −78° C. The mixture was quenched with water (100 mL) and concentrated under reduced pressure. The residue was purified by reverse phase flash chromatography with the following conditions: Column, Spherical C18, 20-40 um, 330 g; Mobile Phase A: water (plus 10 mM ammonium bicarbonate); Mobile Phase B; acetonitrile; Flow rate: 80 mL/min; Gradient: 50%-70% B in 20 min; Detector: UV 254/220 nm. The fractions containing desired product were collected and concentrated under reduced pressure to afford 4-(((tert-butoxycarbonyl)(cyclopropyl)amino)methyl)-2-fluorobenzoic acid as an off-white solid.

Yield 3.00 g (33%). ¹H NMR (400 MHz, DMSO) δ 8.02-7.96 (m, 1H), 7.10 (dd, J=1.6, 8.0 Hz, 1H), 7.04 (dd, J=1.6, 8.0 Hz, 1H), 4.48 (s, 2H), 2.59-2.56 (m, 1H), 1.48 (s, 9H), 0.80-0.75 (m, 2H), 0.70-0.63 (m, 2H). OH proton not observed. m/z: [ESI⁻] 308 (M−H)⁻.

### Synthesis of ethyl 3-(4-(((tert-butoxycarbonyl)(cyclopropyl)amino)methyl)-2-fluorobenzamido)-2-imino-2,3-dihydrobenzo[d]thiazole-6-carboxylate

Compound ethyl 3-(4-(((tert-butoxycarbonyl)(cyclopropyl)amino)methyl)-2-fluorobenzamido)-2-imino-2,3-dihydrobenzo[d]thiazole-6-carboxylate was prepared from 4-(((tert-butoxycarbonyl)(cyclopropyl)amino)methyl)-2-fluorobenzoic acid (3.00 g, 9.70 mmol) and 6-(ethoxycarbonyl)-2-iminobenzo[d]thiazol-3(2H)-aminium 2,4,6-trimethylbenzenesulfonate (3.54 g, 8.09 mmol) following a similar procedure to that described for the synthesis of 2-bromo-N-(3-(piperidin-1-yl)propyl)benzo[d]imidazo[2,1-b]thiazole-7-carboxamide, and was isolated as an off-white solid.

Yield 1.60 g (37%). ¹H NMR (400 MHz, DMSO) δ 8.40 (d, J=1.6 Hz, 1H), 8.25-8.17 (m, 2H), 7.75 (d, J=8.4 Hz, 1H), 7.14 (t, J=8.0 Hz, 1H), 7.09-7.00 (m, 1H), 5.26 (br s, 2H), 4.49 (s, 2H), 4.45 (q, J=7.2 Hz, 2H), 2.60-2.50 (m, 1H), 1.49 (s, 9H), 1.45 (t, J=7.2 Hz, 3H), 0.79-0.75 (m, 2H), 0.69-0.66 (m, 2H). m/z: [ESI⁺] 529 (M+H)⁺.

### ethyl 2-(4-((cyclopropylamino)methyl)-2-fluorophenyl)benzo[4,5]thiazolo[3,2-b][1,2,4]triazole-6-carboxylate

Compound ethyl 2-(4-((cyclopropylamino)methyl)-2-fluorophenyl)benzo[4,5]thiazolo[3,2-b][1,2,4]triazole-6-carboxylate was prepared from ethyl 3-(4-(((tert-butoxycarbonyl)(cyclopropyl)amino)methyl)-2-fluorobenzamido)-2-imino-2,3-dihydrobenzo[d]thiazole-6-carboxylate (1.60 g, 3.03 mmol) following a similar procedure to that described for the synthesis of ethyl 2-(4-((cyclopropylamino)methyl)phenyl)benzo[4,5]thiazolo[3,2-b][1,2,4]triazole-6-carboxylate, and was isolated as an off-white solid.

Yield 1.12 g (90%). ¹H NMR (400 MHz, CDCl₃) δ 8.42 (d, J=1.6 Hz, 1H), 8.23-8.10 (m, 2H), 7.97 (d, J=8.4 Hz, 1H), 7.35-7.28 (m, 2H), 4.42 (q, J=7.2 Hz, 2H), 4.07 (s, 2H), 2.55-2.47 (m, 1H), 1.45 (t, J=7.2 Hz, 3H), 1.04-0.91 (m, 2H), 0.82-0.72 (m, 2H). NH proton not observed. m/z: [ESI⁺] 411 (M+H)⁺.

### ethyl 2-(4-(((tert-butoxycarbonyl)(cyclopropyl)amino)methyl)-2-fluorophenyl)benzo[4,5]thiazolo[3,2-b][1,2,4]triazole-6-carboxylate

Compound ethyl 2-(4-(((tert-butoxycarbonyl)(cyclopropyl)amino)methyl)-2-fluorophenyl)benzo[4,5]thiazolo[3,2-b][1,2,4]triazole-6-carboxylate was prepared from ethyl 2-(4-((cyclopropylamino)methyl)-2-fluorophenyl)benzo[4,5]thiazolo[3,2-b][1,2,4]triazole-6-carboxylate (1.12 g, 2.73 mmol) following a similar procedure to that described for the synthesis of tert-butyl 2-(4-bromophenyl)pyrrolidine-1-carboxylate, and was isolated as an off-white solid.

Yield 1.25 g (90%). ¹H NMR (400 MHz, DMSO) δ 8.90 (d, J=1.6 Hz, 1H), 8.22 (dd, J=1.6, 8.4 Hz, 1H), 8.18-8.10 (m, 2H), 7.26-7.16 (m, 2H), 4.46 (s, 2H), 4.39 (q, J=7.2 Hz, 2H), 2.52-2.49 (m, 1H), 1.42 (s, 9H), 1.38 (t, J=7.2 Hz, 3H), 0.74-0.67 (m, 2H), 0.66-0.59 (m, 2H). m/z: [ESI⁺] 511 (M+H)⁺.

### 2-(4-(((tert-butoxycarbonyl)(cyclopropyl)amino)methyl)-2-fluorophenyl)benzo[4,5]thiazolo[3,2-b][1,2,4]triazole-6-carboxylic acid

Compound 2-(4-(((tert-butoxycarbonyl)(cyclopropyl)amino)methyl)-2-fluorophenyl)benzo[4,5]thiazolo[3,2-b][1,2,4]triazole-6-carboxylic acid was prepared from ethyl 2-(4-(((tert-butoxycarbonyl)(cyclopropyl)amino)methyl)-2-fluorophenyl)benzo[4,5]thiazolo[3,2-b] [1,2,4]triazole-6-carboxylate (1.45 g, 2.84 mmol) following a similar procedure to that described for the synthesis of 4-(7-bromobenzo[d]imidazo[2,1-b]thiazol-2-yl)-3-(trifluoromethyl)benzoic acid, and was isolated as an off-white solid.

Yield 0.92 g (67%). ¹H NMR (400 MHz, DMSO) δ 13.28 (br s, 1H), 8.86 (d, J=1.6 Hz, 1H), 8.21 (dd, J=1.6, 8.4 Hz, 1H), 8.18-8.10 (m, 2H), 7.25-7.17 (m, 2H), 4.46 (s, 2H), 2.54-2.45 (m, 1H), 1.42 (s, 9H), 0.76-0.66 (m, 2H), 0.66-0.60 (m, 2H). m/z: [ESI⁺] 483 (M+H)⁺.

### tert-butyl cyclopropyl(4-(6-((3-(diethylamino)propyl)carbamoyl)benzo[4,5]thiazolo[3,2-b][1,2,4]triazol-2-yl)-3-fluorobenzyl)carbamate

Compound tert-butyl cyclopropyl(4-(6-((3-(diethylamino)propyl)carbamoyl)benzo[4,5]thiazolo[3,2-b][1,2,4]triazol-2-yl)-3-fluorobenzyl)carbamate was prepared from 2-(4-(((tert-butoxycarbonyl)(cyclopropyl)amino)methyl)-2-fluorophenyl)benzo[4,5]thiazolo[3,2-b][1,2,4]triazole-6-carboxylic acid (200 mg, 0.414 mmol) and N′,N¹-diethylpropane-1,3-diamine (107 mg, 0.822 mmol) following a similar procedure to that described for the synthesis of 2-bromo-N-(3-(piperidin-1-yl)propyl)benzo[d]imidazo[2,1-b]thiazole-7-carboxamide, and was isolated as a white solid.

Yield 0.18 g (73%). ¹H NMR (400 MHz, DMSO) δ 8.83 (t, J=5.6 Hz, 1H), 8.69 (s, 1H), 8.18 (d, J=8.8 Hz, 1H), 8.15-8.10 (m, 2H), 7.25-7.18 (m, 2H), 4.46 (s, 2H), 3.46-3.36 (m, 2H), 2.58-2.53 (m, 7H), 1.95-1.87 (m, 2H), 1.42 (s, 9H), 1.20 (t, J=7.2 Hz, 6H), 0.75-0.67 (m, 2H), 0.66-0.61 (m, 2H). m/z: [ESI⁺]595 (M+H)⁺.

### tert-butyl cyclopropyl(3-fluoro-4-(6-((3-(piperidin-1-yl)propyl)carbamoyl)benzo[4,5]thiazolo[3,2-b][1,2,4]triazol-2-yl)benzyl)carbamate

Compound tert-butyl cyclopropyl(3-fluoro-4-(6-((3-(piperidin-1-yl)propyl)carbamoyl)benzo[4,5]thiazolo[3,2-b][1,2,4]triazol-2-yl)benzyl)carbamate was prepared from 2-(4-(((tert-butoxycarbonyl)(cyclopropyl)amino)methyl)-2-fluorophenyl)benzo[4,5]thiazolo[3,2-b][1,2,4]triazole-6-carboxylic acid (300 mg, 0.622 mmol) and 3-(piperidin-1-yl)propan-1-amine (133 mg, 0.935 mmol) following a similar procedure to that described for the synthesis of 2-bromo-N-(3-(piperidin-1-yl)propyl)benzo[d]imidazo[2,1-b]thiazole-7-carboxamide, and was isolated as a white solid.

Yield 300 mg (79%). ¹H NMR (400 MHz, DMSO) δ 8.78 (t, J=5.6 Hz, 1H), 8.68 (s, 1H), 8.17-8.11 (m, 3H), 7.25-7.17 (m, 2H), 4.46 (s, 2H), 3.40-3.31 (m, 2H), 2.74-2.63 (m, 6H), 2.58-2.53 (m, 1H), 1.87-1.77 (m, 2H), 1.64-1.57 (m, 4H), 1.49-1.44 (m, 2H), 1.42 (s, 9H), 0.74-0.68 (m, 2H), 0.67-0.60 (m, 2H). m/z: [ESI⁺] 607 (M+H)⁺.

### tert-butyl (1-(4-(6-((3-(diethylamino)propyl)carbamoyl)benzo[4,5]thiazolo[3,2-b][1,2,4]triazol-2-yl)phenyl)cyclopropyl)carbamate

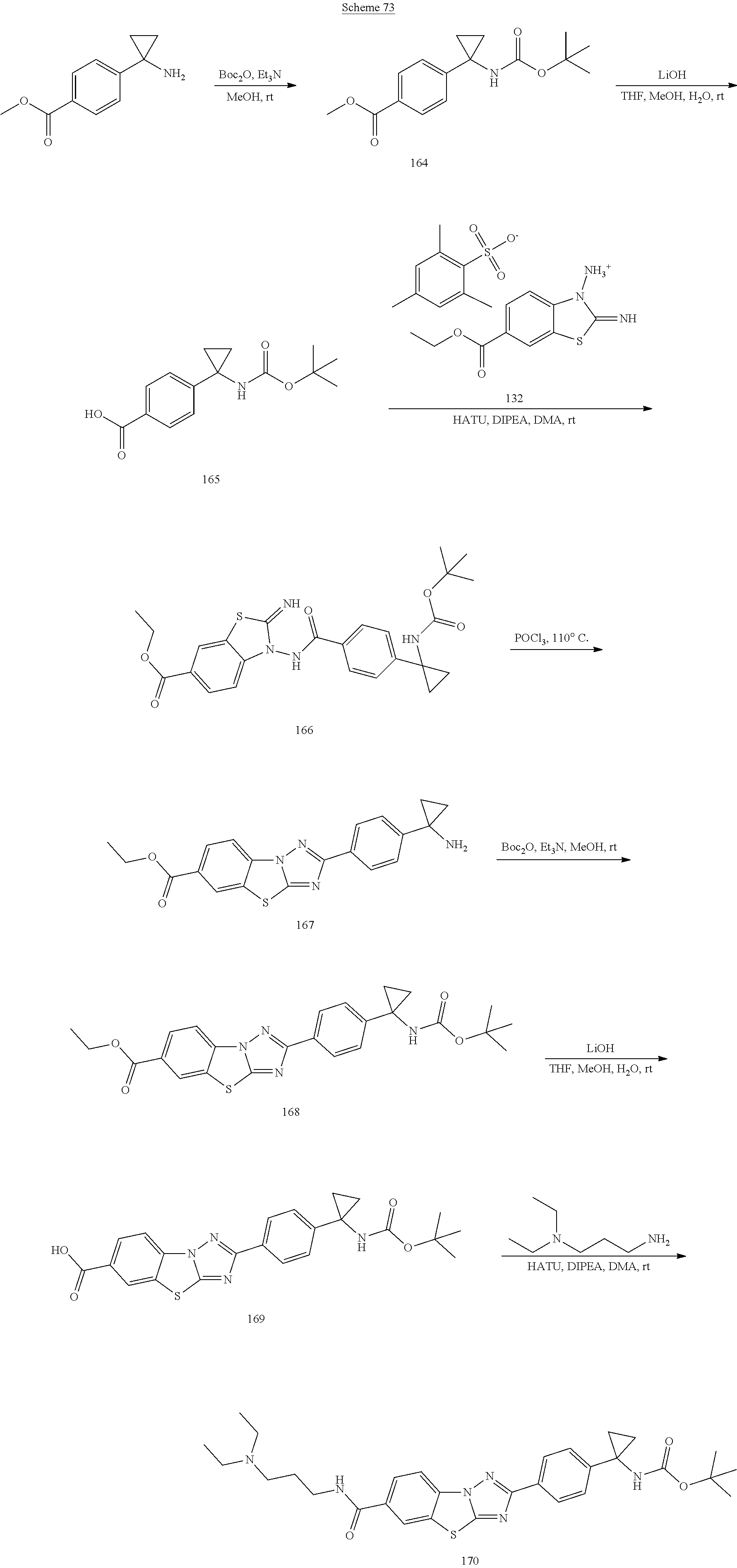

### methyl 4-(1-((tert-butoxycarbonyl)amino)cyclopropyl)benzoate

Compound methyl 4-(1-((tert-butoxycarbonyl)amino)cyclopropyl)benzoate was prepared from methyl 4-(1-aminocyclopropyl)benzoate hydrochloride (5.00 g, 21.96 mmol) following a similar procedure to that described for the synthesis of tert-butyl 2-(4-bromophenyl)pyrrolidine-1-carboxylate, and was isolated as an off-white solid.

Yield 5.27 g (82%). ¹H NMR (400 MHz, CDCl₃) δ 7.98 (d, J=8.4 Hz, 2H), 7.25 (d, J=8.4 Hz, 2H), 5.32 (br s, 1H) 3.92 (s, 3H), 1.47 (s, 9H), 1.40-1.35 (m, 2H), 1.33-1.28 (m, 2H). m/z: [ESI⁻] 290 (M−H)⁻.

### 4-(1-((tert-butoxycarbonyl)amino)cyclopropyl)benzoic acid

Compound 4-(1-((tert-butoxycarbonyl)amino)cyclopropyl)benzoic acid was prepared from methyl 4-(1-((tert-butoxycarbonyl)amino)cyclopropyl)benzoate (5.27 g, 18.09 mmol) following a similar procedure to that described for the synthesis of 4-(7-bromobenzo[d]imidazo[2,1-b]thiazol-2-yl)-3-(trifluoromethyl)benzoic acid, and was isolated as an off-white solid.

Yield 3.48 g (69%). ¹H NMR (400 MHz, CD₃OD) δ 7.95 (d, J=8.4 Hz, 2H), 7.28 (d, J=8.4 Hz, 2H), 1.47 (s, 9H), 1.39-1.25 (m, 4H). NH and CO₂H protons not observed. m/z: [ESI] 276 (M−H)⁻.

### ethyl 3-(4-(1-((tert-butoxycarbonyl)amino)cyclopropyl)benzamido)-2-imino-2,3-dihydrobenzo[d]thiazole-6-carboxylate

Compound ethyl 3-(4-(1-((tert-butoxycarbonyl)amino)cyclopropyl)benzamido)-2-imino-2,3-dihydrobenzo[d]thiazole-6-carboxylate was prepared from 4-(1-((tert-butoxycarbonyl)amino)cyclopropyl)benzoic acid (3.48 g, 12.55 mmol) and 6-(ethoxycarbonyl)-2-iminobenzo[d]thiazol-3(2H)-aminium 2,4,6-trimethylbenzenesulfonate (4.22 g, 9.65 mmol) following a similar procedure to that described for the synthesis of 2-bromo-N-(3-(piperidin-1-yl)propyl)benzo[d]imidazo[2,1-b]thiazole-7-carboxamide, and was isolated as a white solid.

Yield 3.62 g (76%). ¹H NMR (400 MHz, CDCl₃) δ 8.21 (s, 1H), 8.15 (d, J=8.0 Hz, 2H), 8.10 (d, J=8.4 Hz, 1H), 7.61 (d, J=8.4 Hz, 1H), 7.12 (d, J=8.0 Hz, 2H), 5.45 (br s, 1H), 5.19 (br s, 2H) 4.41 (q, J=7.2 Hz, 2H), 1.49 (s, 9H), 1.44 (t, J=7.2 Hz, 3H), 1.39-1.34 (m, 2H), 1.30-1.25 (m, 2H). m/z: [ESI⁺]497 (M+H)⁺.

### ethyl 2-(4-(1-aminocyclopropyl)phenyl)benzo[4,5]thiazolo[3,2-b][1,2,4]triazole-6-carboxylate

Compound ethyl 2-(4-(1-aminocyclopropyl)phenyl)benzo[4,5]thiazolo[3,2-b][1,2,4]triazole-6-carboxylate was prepared from ethyl 3-(4-(1-((tert-butoxycarbonyl)amino)cyclopropyl)benzamido)-2-imino-2,3-dihydrobenzo[d]thiazole-6-carboxylate (3.62 g, 7.29 mmol) following a similar procedure to that described for the synthesis of ethyl 2-(4-((cyclopropylamino)methyl)phenyl)benzo[4,5]thiazolo[3,2-b][1,2,4]triazole-6-carboxylate, and was isolated as an off-white solid.

Yield 0.76 g (28%). ¹H NMR (400 MHz, CDCl₃) δ 8.52 (s, 1H), 8.29 (d, J=8.4 Hz, 1H), 8.19 (d, J=8.0 Hz, 2H), 8.06 (d, J=8.4 Hz, 1H), 7.43 (d, J=8.0 Hz, 2H), 4.47 (q, J=7.2 Hz, 2H), 1.47 (t, J=7.2 Hz, 3H), 1.22-1.16 (m, 2H), 1.14-1.06 (m, 2H). Aliphatic NH₂ protons not observed m/z: [ESI⁺] 379 (M+H)⁺.

### ethyl 2-(4-(1-((tert-butoxycarbonyl)amino)cyclopropyl)phenyl)benzo[4,5]thiazolo[3,2-b][1,2,4]triazole-6-carboxylate

Compound ethyl 2-(4-(1-((tert-butoxycarbonyl)amino)cyclopropyl)phenyl)benzo[4,5]thiazolo[3,2-b][1,2,4]triazole-6-carboxylate was prepared from ethyl 2-(4-(1-aminocyclopropyl)phenyl)benzo[4,5]thiazolo[3,2-b][1,2,4]triazole-6-carboxylate (200 mg, 0.528 mmol) following a similar procedure to that described for the synthesis of tert-butyl 2-(4-bromophenyl)pyrrolidine-1-carboxylate, and was isolated as an off-white solid.

Yield 161 mg (64%). ¹H NMR (400 MHz, CDCl₃) δ 8.53 (d, J=1.6 Hz, 1H), 8.29 (d, J=8.4 Hz, 1H), 8.18 (d, J=8.0 Hz, 2H), 8.06 (dd, J=1.6, 8.4 Hz, 1H), 7.34 (d, J=8.0 Hz, 2H), 5.33 (br s, 1H) 4.47 (q, J=7.2 Hz, 2H), 1.49 (s, 9H), 1.46 (t, J=7.2 Hz, 3H) 1.36-1.28 (m, 4H). m/z: [ESI] 479 (M+H)⁺.

### 2-(4-(1-((tert-butoxycarbonyl)amino)cyclopropyl)phenyl)benzo[4,5]thiazolo[3,2-b][1,2,4]triazole-6-carboxylic acid

Compound 2-(4-(1-((tert-butoxycarbonyl)amino)cyclopropyl)phenyl)benzo[4,5]thiazolo[3,2-b][1,2,4]triazole-6-carboxylic acid was prepared from ethyl 2-(4-(1-((tert-butoxycarbonyl)amino)cyclopropyl)phenyl)benzo[4,5]thiazolo[3,2-b][1,2,4]triazole-6-carboxylate (700 mg, 1.463 mmol) following a similar procedure to that described for the synthesis of 4-(7-bromobenzo[d]imidazo[2,1-b]thiazol-2-yl)-3-(trifluoromethyl)benzoic acid, and was isolated as a white solid.

Yield 630 mg (96%). ¹H NMR (400 MHz, DMSO) δ 8.67 (d, J=1.6 Hz, 1H), 8.21-8.12 (m, 2H), 8.07 (d, J=8.0 Hz, 2H), 7.98 (d, J=8.4 Hz, 1H), 7.28 (d, J=8.0 Hz, 2H), 1.41 (s, 9H) 1.27-1.14 (m, 4H). OH proton not observed. m/z: [ESI⁺] 451 (M+H)⁺.

### tert-butyl (1-(4-(6-((3-(diethylamino)propyl)carbamoyl)benzo[4,5]thiazolo[3,2-b][1,2,4]triazol-2-yl)phenyl)cyclopropyl)carbamate

Compound tert-butyl (1-(4-(6-((3-(diethylamino)propyl)carbamoyl)benzo[4,5]thiazolo[3,2-b][1,2,4]triazol-2-yl)phenyl)cyclopropyl)carbamate was prepared from 2-(4-(1-((tert-butoxycarbonyl)amino)cyclopropyl)phenyl)benzo[4,5]thiazolo[3,2-b][1,2,4]triazole-6-carboxylic acid (80 mg, 0.178 mmol) and N′,N¹-diethylpropane-1,3-diamine (18 mg, 0.138 mmol) following a similar procedure to that described for the synthesis of 2-bromo-N-(3-(piperidin-1-yl)propyl)benzo[d]imidazo[2,1-b]thiazole-7-carboxamide, and was isolated as a white solid.

Yield 75 mg (97%). ¹H NMR (400 MHz, CDCl₃) δ 9.19 (br s, 1H), 8.34 (d, J=1.6 Hz, 1H), 8.14 (d, J=8.0 Hz, 2H), 8.01 (dd, J=1.6, 8.4 Hz, 1H), 7.94 (dd, J=1.6, 8.4 Hz, 1H), 7.31 (d, J=8.0 Hz, 2H), 5.41 (br s, 1H), 3.68-3.60 (m, 2H), 2.73-2.68 (m, 2H), 2.68-2.60 (m, 4H), 1.86-1.78 (m, 2H), 1.48 (s, 9H) 1.37-1.28 (m, 4H), 1.08 (t, J=7.2 Hz, 6H). m/z: [ESI⁺] 563 (M+H)⁺.

### tert-butyl (1-(4-(6-((3-(piperidin-1-yl)propyl)carbamoyl)benzo[4,5]thiazolo[3,2-b][1,2,4]triazol-2-yl)phenyl)cyclopropyl)carbamate

Compound tert-butyl (1-(4-(6-((3-(piperidin-1-yl)propyl)carbamoyl)benzo[4,5]thiazolo[3,2-b][1,2,4]triazol-2-yl)phenyl)cyclopropyl)carbamate was prepared from 2-(4-(1-((tert-butoxycarbonyl)amino)cyclopropyl)phenyl)benzo[4,5]thiazolo[3,2-b][1,2,4]triazole-6-carboxylic acid (300 mg, 0.666 mmol) and 3-(piperidin-1-yl)propan-1-amine (122 mg, 0.858 mmol) following a similar procedure to that described for the synthesis of 2-bromo-N-(3-(piperidin-1-yl)propyl)benzo[d]imidazo[2,1-b]thiazole-7-carboxamide, and was isolated as a white solid.

Yield 209 mg (55%). ¹H NMR (400 MHz, CDCl₃) δ 9.14 (br s, 1H), 8.40 (s, 1H), 8.16 (d, J=8.0 Hz, 2H), 8.09-7.96 (m, 2H), 7.32 (d, J=8.0 Hz, 2H), 5.38 (br s, 1H), 3.68-3.59 (m, 2H), 2.65-2.60 (m, 2H), 2.60-2.41 (m, 4H), 1.91-1.82 (m, 2H), 1.70-1.61 (m, 4H), 1.55-1.52 (m, 2H), 1.49 (s, 9H), 1.43-1.30 (m, 4H). m/z: [ESI⁺] 575 (M+H)⁺.

### tert-butyl (4-(6-((3-(diethylamino)propyl)carbamoyl)benzo[4,5]thiazolo[3,2-b][1,2,4]triazol-2-yl)benzyl)carbamate

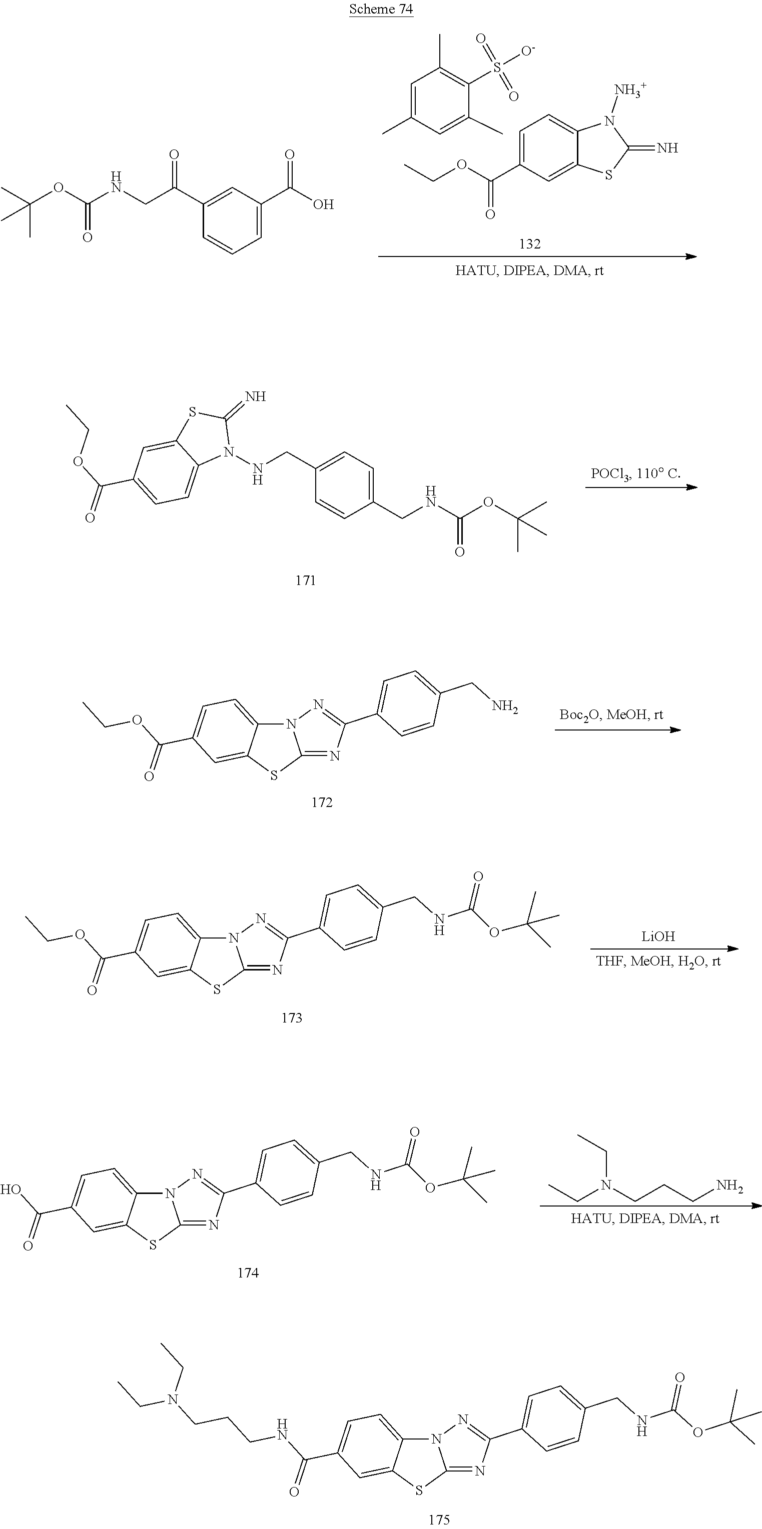

### ethyl 3-(4-(((tert-butoxycarbonyl)amino)methyl)benzamido)-2-imino-2,3-dihydrobenzo[d]thiazole-6-carboxylate

Compound ethyl 3-(4-(((tert-butoxycarbonyl)amino)methyl)benzamido)-2-imino-2,3-dihydrobenzo[d]thiazole-6-carboxylate was prepared from 6-(ethoxycarbonyl)-2-iminobenzo[d]thiazol-3(2H)-aminium 2,4,6-trimethylbenzenesulfonate (2.60 g, 5.94 mmol) and 4-(((tert-butoxycarbonyl)amino)methyl)benzoic acid (2.24 g, 8.91 mmol) following a similar procedure to that described for the synthesis of 2-bromo-N-(3-(piperidin-1-yl)propyl)benzo[d]imidazo[2,1-b]thiazole-7-carboxamide, and was isolated as a white solid.

Yield 2.57 g (92%). ¹H NMR (400 MHz, DMSO) δ 8.54 (d, J=1.6 Hz, 1H), 8.34 (d, J=8.0 Hz, 2H), 8.13 (dd, J=1.6, 8.4 Hz, 1H), 7.76 (d, J=8.4 Hz, 1H), 7.48 (t, J=6.4 Hz, 1H), 7.39 (d, J=8.0 Hz, 2H), 6.44 (br s, 2H), 4.36 (q, J=7.2 Hz, 2H), 4.23 (d, J=6.4 Hz, 2H), 1.42 (s, 9H), 1.36 (q, J=7.2 Hz, 3H). m/z: [ESI⁺] 471 (M+H)⁺.

### ethyl 2-(4-(aminomethyl)phenyl)benzo[4,5]thiazolo[3,2-b][1,2,4]triazole-6-carboxylate

Compound ethyl 2-(4-(aminomethyl)phenyl)benzo[4,5]thiazolo[3,2-b][1,2,4]triazole-6-carboxylate was prepared from ethyl 3-(4-(((tert-butoxycarbonyl)amino)methyl)benzamido)-2-imino-2,3-dihydrobenzo[d]thiazole-6-carboxylate (1.75 g, 3.72 mmol) following a similar procedure to that described for the synthesis of ethyl 2-(4-((cyclopropylamino)methyl)phenyl)benzo[4,5]thiazolo[3,2-b][1,2,4]triazole-6-carboxylate, and was isolated as an off-white solid.

Yield 0.80 g (61%). ¹H NMR (400 MHz, CDCl₃) δ 8.87 (s, 1H), 8.34-8.04 (m, 3H), 7.78-7.36 (m, 3H), 4.41-4.36 (m, 2H), 4.11-4.39 (m, 2H), 1.42-1.27 (m, 3H). NH₂ protons not observed, m/z: [ESI⁺] 353 (M+H)⁺.

### ethyl 2-(4-(((tert-butoxycarbonyl)amino)methyl)phenyl)benzo[4,5]thiazolo[3,2-b][1,2,4]triazole-6-carboxylate

Compound ethyl 2-(4-(((tert-butoxycarbonyl)amino)methyl)phenyl)benzo[4,5]thiazolo[3,2-b][1,2,4]triazole-6-carboxylate was prepared from ethyl 2-(4-(aminomethyl)phenyl)benzo[4,5]thiazolo[3,2-b][1,2,4]triazole-6-carboxylate (800 mg, 2.270 mmol) following a similar procedure to that described for the synthesis of tert-butyl 2-(4-bromophenyl)pyrrolidine-1-carboxylate, and was isolated as an off-white solid.

Yield 700 mg (68%). ¹H NMR (400 MHz, DMSO) δ 8.88 (d, J=1.6 Hz, 1H), 8.21 (dd, J=1.6, 8.4 Hz, 1H), 8.15-8.10 (m, 3H), 7.47 (t, J=6.4, 1H), 7.42 (d, J=8.0 Hz, 2H), 4.39 (q, J=7.2, 2H), 4.22 (d, J=6.4 Hz, 2H), 1.42 (s, 9H), 1.38 (t, J=7.2 Hz, 3H). m/z: [ESI⁺] 453 (M+H)⁺.

### 2-(4-(((tert-butoxycarbonyl)amino)methyl)phenyl)benzo[4,5]thiazolo[3,2-b][1,2,4]triazole-6-carboxylic acid

Compound 2-(4-(((tert-butoxycarbonyl)amino)methyl)phenyl)benzo[4,5]thiazolo[3,2-b][1,2,4]triazole-6-carboxylic acid was prepared from ethyl 2-(4-(((tert-butoxycarbonyl)amino)methyl)phenyl)benzo[4,5]thiazolo[3,2-b][1,2,4]triazole-6-carboxylate (600 mg, 1.326 mmol) following a similar procedure to that described for the synthesis of 4-(7-bromobenzo[d]imidazo[2,1-b]thiazol-2-yl)-3-(trifluoromethyl)benzoic acid, and was isolated as a white solid.

Yield 180 mg (34%). ¹H NMR (400 MHz, DMSO) δ 13.29 (br s, 1H), 8.85 (d, J=1.6 Hz, 1H), 8.21 (dd, J=1.6, 8.4 Hz, 1H), 8.15-8.09 (m, 3H), 7.47 (t, J=6.4 Hz, 1H), 7.42 (d, J=8.0 Hz, 2H), 4.21 (d, J=6.4 Hz, 2H), 1.42 (s, 9H), m/z: [ESI⁺] 425 (M+H)⁺.

### tert-butyl (4-(6-((3-(diethylamino)propyl)carbamoyl)benzo[4,5]thiazolo[3,2-b][1,2,4]triazol-2-yl)benzyl)carbamate

Compound tert-butyl (4-(6-((3-(diethylamino)propyl)carbamoyl)benzo[4,5]thiazolo[3,2-b][1,2,4]triazol-2-yl)benzyl)carbamate was prepared from 2-(4-(((tert-butoxycarbonyl)amino)methyl)phenyl)benzo[4,5]thiazolo[3,2-b][1,2,4]triazole-6-carboxylic acid (300 mg, 0.707 mmol) and N′,N¹-diethylpropane-1,3-diamine (122 mg, 0.937 mmol)) following a similar procedure to that described for the synthesis of 2-bromo-N-(3-(piperidin-1-yl)propyl)benzo[d]imidazo[2,1-b]thiazole-7-carboxamide, which was isolated as a white solid and was used directly in next step.

Yield 209 mg (55%). m/z: [ESI⁺] 537 (M+H)⁺.

### tert-butyl (4-(6-((3-(piperidin-1-yl)propyl)carbamoyl)benzo[4,5]thiazolo[3,2-b][1,2,4]triazol-2-yl)benzyl)carbamate

Compound tert-butyl (4-(6-((3-(piperidin-1-yl)propyl)carbamoyl)benzo[4,5]thiazolo[3,2-b][1,2,4]triazol-2-yl)benzyl)carbamate was prepared from 2-(4-(((tert-butoxycarbonyl)amino)methyl)phenyl)benzo[4,5]thiazolo[3,2-b][1,2,4]triazole-6-carboxylic acid (360 mg, 0.848 mmol) and 3-(piperidin-1-yl)propan-1-amine (180 mg, 1.265 mmol) following a similar procedure to that described for the synthesis of 2-bromo-N-(3-(piperidin-1-yl)propyl)benzo[d]imidazo[2,1-b]thiazole-7-carboxamide, and was isolated as a white solid.

Yield 300 mg (64%). ¹H NMR (400 MHz, DMSO) δ 8.70 (t, J=5.6 Hz, 1H), 8.65 (s, 1H), 8.16-8.10 (m, 4H), 7.46 (d, J=6.0 Hz, 1H), 7.42 (d, J=8.0 Hz, 2H), 4.22 (d, J=6.0 Hz, 2H), 3.37-3.34 (m, 2H), 2.38-2.28 (m, 6H), 1.75-1.65 (m, 2H), 1.54-1.45 (m, 4H), 1.42 (s, 9H), 1.40-1.35 (m, 2H). m/z: [ESI⁺]549 (M+H)⁺.

### 2-(4-(methylcarbamoyl)phenyl)benzo[4,5]thiazolo[3,2-b][1,2,4]triazole-6-carboxylic acid

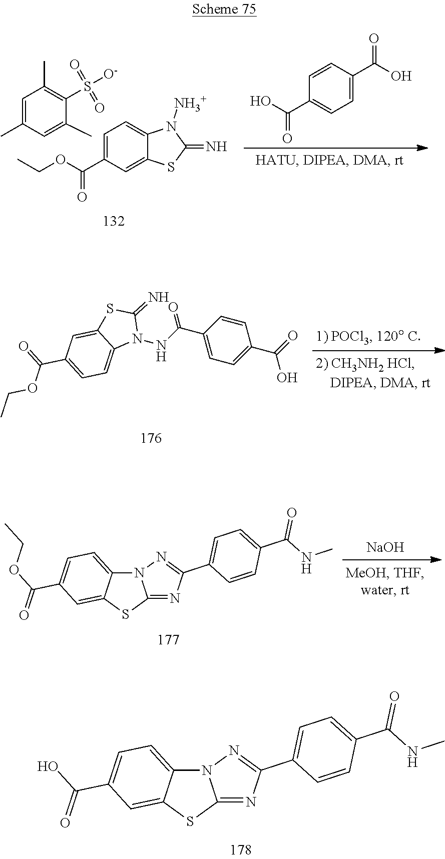

### 4-((6-(ethoxycarbonyl)-2-iminobenzo[d]thiazol-3(2H)-yl)carbamoyl)benzoic acid

Compound 4-((6-(ethoxycarbonyl)-2-iminobenzo[d]thiazol-3(2H)-yl)carbamoyl)benzoic acid was prepared from 6-(ethoxycarbonyl)-2-iminobenzo[d]thiazol-3(2H)-aminium 2,4,6-trimethylbenzenesulfonate (1.50 g, 3.43 mmol) and benzene-1,4-dicarboxylic acid (0.85 g, 5.12 mmol) following a similar procedure to that described for the synthesis of 2-bromo-N-(3-(piperidin-1-yl)propyl)benzo[d]imidazo[2,1-b]thiazole-7-carboxamide, and was isolated as an off-white solid.

Yield 1.2 g (91%). ¹H NMR (400 MHz, DMSO) δ 13.30 (br s, 1H), 8.67 (s, 1H), 8.47 (d, J=8.0 Hz, 2H), 8.23 (d, J=8.8 Hz, 1H), 8.09 (d, J=8.0 Hz, 2H), 7.78 (d, J=8.8 Hz, 1H), 4.35 (q, J=7.2 Hz, 2H), 1.35 (t, J=7.2 Hz, 3H). Two NH protons not observed. m/z: [ESI⁺] 386 (M+H)⁺.

### ethyl 2-(4-(methylcarbamoyl)phenyl)benzo[4,5]thiazolo[3,2-b][1,2,4]triazole-6-carboxylate

A solution of 4-((6-(ethoxycarbonyl)-2-iminobenzo[d]thiazol-3(2H)-yl)carbamoyl)benzoic acid (1.20 g, 3.11 mmol) in phosphorus(V) oxychloride (20 mL) was stirred for 16 h at 120° C. under a nitrogen atmosphere. The mixture was cooled to room temperature and was concentrated under reduced pressure. The residue was diluted with N,N-dimethylacetamide (10 mL) and was added drop-wise to a solution of methanamine hydrochloride (0.63 g, 9.34 mmol) and N-ethyl-N-isopropylpropan-2-amine (2.41 g, 18.68 mmol) in N,N-dimethylacetamide (5 mL). The resulting mixture was stirred for additional 16 h at room temperature under a nitrogen atmosphere. The reaction mixture was quenched with water (50 mL). The precipitated solids were collected by filtration, washed with water (3×10 mL) and oven dried to afford ethyl 2-(4-(methylcarbamoyl)phenyl)benzo[4,5]thiazolo[3,2-b][1,2,4]triazole-6-carboxylate as an off-white solid.

Yield 0.65 g (55%). ¹H NMR (400 MHz, DMSO) δ 8.89 (s, 1H), 8.58 (m, 1H), 8.26-8.18 (m, 3H), 8.15 (d, J=8.4 Hz, 1H), 8.00 (d, J=8.0 Hz, 2H), 4.38 (q, J=7.2 Hz, 2H), 2.82 (d, J=4.4 Hz, 3H), 1.37 (t, J=7.2 Hz, 3H). m/z: [ESI⁺] 381 (M+H)⁺.

### 2-(4-(methylcarbamoyl)phenyl)benzo[4,5]thiazolo[3,2-b][1,2,4]triazole-6-carboxylic acid

Compound 2-(4-(methylcarbamoyl)phenyl)benzo[4,5]thiazolo[3,2-b][1,2,4]triazole-6-carboxylic acid was prepared from ethyl 2-(4-(methylcarbamoyl)phenyl)benzo[4,5]thiazolo[3,2-b][1,2,4]triazole-6-carboxylate (650 mg, 1.709 mmol) following a similar procedure to that described for the synthesis of 4-(7-bromobenzo[d]imidazo[2,1-b]thiazol-2-yl)-3-(trifluoromethyl)benzoic acid, and was isolated as an off-white solid.

Yield 500 mg (83%). ¹H NMR (400 MHz, DMSO) δ 13.33 (br s, 1H), 8.87 (d, J=1.6 Hz, 1H), 8.58 (q, J=4.4 Hz, 1H), 8.27-8.19 (m, 3H), 8.15 (d, J=8.8 Hz, 1H), 8.01 (d, J=8.8 Hz, 2H), 2.82 (d, J=4.4 Hz, 3H). m/z: [ESI⁺] 353 (M+H)⁺.

### Example 4

### Synthetic Details for Additional Intermediates of Compounds of the Invention (Schemes 76-90)

### Synthesis of (S)-2-(2-fluoro-4-(tetrahydrofuran-2-yl)phenyl)benzo[d]imidazo[2,1-b]thiazole-7-carboxylic acid (Intermediate C1, Scheme 76)

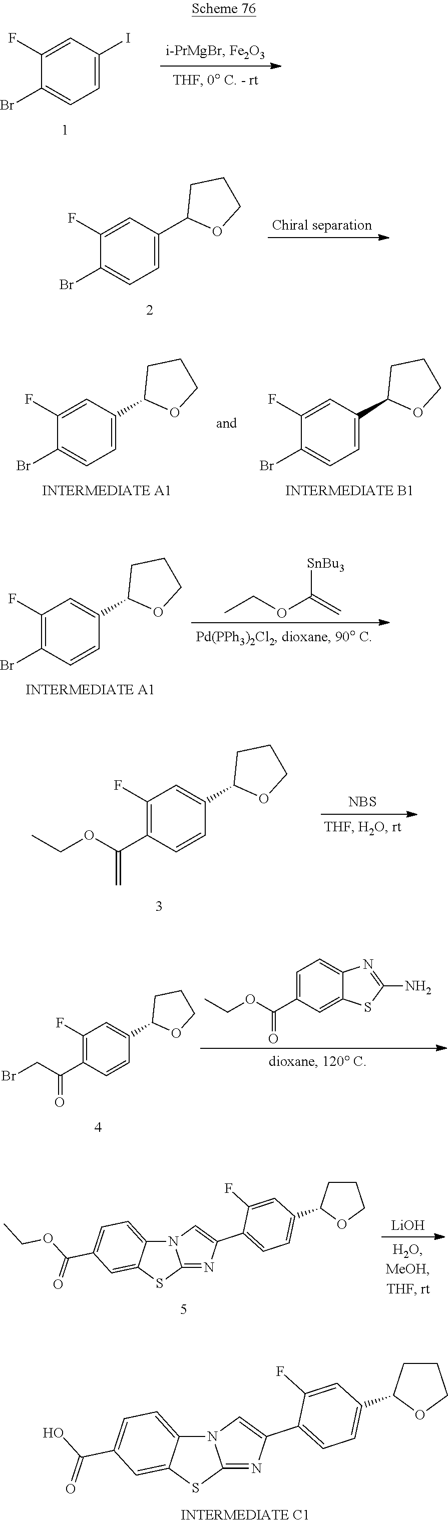

### Synthesis of 2-(4-bromo-3-fluorophenyl)tetrahydrofuran (Intermediate 2, Scheme 76)

To a stirred solution of 1-bromo-2-fluoro-4-iodobenzene (15.00 g, 49.85 mmol) in tetrahydrofuran (300 mL) was added isopropylmagnesium bromide (1 N in tetrahydrofuran, 50 mL, 50.00 mmol) dropwise at 0° C. under a nitrogen atmosphere. The reaction solution was stirred for 1 h at 0° C. under a nitrogen atmosphere. To the above solution was added a solution of dry iron(iii) oxide (796 mg, 4.985 mmol) in tetrahydrofuran (20 mL) dropwise over 5 min at 0° C. The resulting mixture was stirred for additional 2 h at room temperature. The resulting mixture was filtered. The filter cake was washed with ethyl acetate (3×10 mL). The combined filtrates were concentrated under reduced pressure. The residue was purified by silica gel column chromatography, eluted with 1%-100% ethyl acetate in petroleum ether to afford 2-(4-bromo-3-fluorophenyl)tetrahydrofuran as a white solid.

Yield: 1.30 g (11%). ¹H NMR (400 MHz, CDCl₃) δ 7.52 (dd, J=6.8, 8.4 Hz, 1H), 7.18 (dd, J=2.0, 9.6 Hz, 1H), 7.04 (dd, J=2.0, 8.4 Hz, 1H), 4.74 (dd, J=4.4, 7.6 Hz, 1H), 3.80-3.65 (m, 2H), 1.92-1.79 (m, 2H), 1.75-1.66 (m, 2H). m/z: [ESI] 261, 263 (M−H+18)⁻.

### Synthesis of (S)-2-(4-bromo-3-fluorophenyl)tetrahydrofuran and (R)-2-(4-bromo-3-fluorophenyl)tetrahydrofuran (Intermediates A1 and B1, Scheme 76)

2-(4-bromo-3-fluorophenyl)tetrahydrofuran (1.30 g, 5.30 mmol) was separated by Prep-Chiral-HPLC with the following conditions: Column: Lux 5 um Cellulose-4, 2.12×25 cm, 5 um; Mobile Phase A: Hexane (0.5% 2 N NH₃-Methanol), Mobile Phase B: Propan-2-ol; Flow rate: 20 mL/min; Gradient: 15% B to 15% B in 9 min; Wave Length: 254/220 nm; RT1 (min): 7.79; RT2 (min): 8.98; Sample Solvent: Methanol. The faster eluting peak was collected and concentrated under reduced pressure to afford (S)-2-(4-bromo-3-fluorophenyl)tetrahydrofuran as an off-white solid.

Yield 400 mg (31%). ¹H NMR (400 MHz, CDCl₃) δ 7.52 (dd, J=6.8, 8.4 Hz, 1H), 7.18 (dd, J=2.0, 9.6 Hz, 1H), 7.04 (dd, J=2.0, 8.4 Hz, 1H), 4.74 (dd, J=4.4, 7.6 Hz, 1H), 3.80-3.65 (m, 2H), 1.92-1.79 (m, 2H), 1.75-1.66 (m, 2H). m/z: [ESI] 261, 263 (M−H+18)⁻.

The slower eluting peak was collected and concentrated under reduced pressure to afford (R)-2-(4-bromo-3-fluorophenyl)tetrahydrofuran as an off-white solid.

Yield 450 mg (35%). ¹H NMR (400 MHz, CDCl₃) δ 7.52 (dd, J=6.8, 8.4 Hz, 1H), 7.18 (dd, J=2.0, 9.6 Hz, 1H), 7.04 (dd, J=2.0, 8.4 Hz, 1H), 4.74 (dd, J=4.4, 7.6 Hz, 1H), 3.80-3.65 (m, 2H), 1.92-1.79 (m, 2H), 1.75-1.66 (m, 2H). m/z: [ESI] 261, 263 (M−H+18)⁻.

### Synthesis of (S)-2-(4-(1-ethoxyvinyl)-3-fluorophenyl)tetrahydrofuran (Intermediate 3, Scheme 76)

To a stirred solution of (S)-2-(4-bromo-3-fluorophenyl)tetrahydrofuran (300 mg, 1.224 mmol) and tributyl(1-ethoxyethenyl)stannane (663 mg, 1.836 mmol) in dioxane (6 mL) was added bis(triphenylphosphine)palladium(II) chloride (86 mg, 0.123 mmol) at room temperature under a nitrogen atmosphere. The reaction solution was stirred for 2 h at 90° C. under a nitrogen atmosphere. The resulting solution was allowed to cool down to room temperature, diluted with water (10 mL) and extracted with ethyl acetate (3×10 mL). The combined organic layers were dried over anhydrous sodium sulfate. After filtration, the filtrate was concentrated under reduced pressure to afford (S)-2-(4-(1-ethoxyvinyl)-3-fluorophenyl)tetrahydrofuran as a brown oil, which was unstable and was used in next step directly without further purification.

Yield 300 mg (crude). m/z: [ESI⁺] 255 (M+H+18)⁺. ¹HNMR not be ran.

### Synthesis of (S)-2-bromo-1-(2-fluoro-4-(tetrahydrofuran-2-yl)phenyl)ethan-1-one (Intermediate 4, Scheme 76)

To a stirred solution of (S)-2-(4-(1-ethoxyvinyl)-3-fluorophenyl)tetrahydrofuran (300 mg, 1.270 mmol) in tetrahydrofuran (6 mL) and water (1 mL) was added 1-bromopyrrolidine-2,5-dione (226 mg, 1.270 mmol) in portions at room temperature under a nitrogen atmosphere. The reaction solution was stirred for 1 h at room temperature under a nitrogen atmosphere. The resulting mixture was concentrated under reduced pressure. The residue was purified by silica gel column chromatography, eluted with 1%-50% ethyl acetate in petroleum ether to afford (S)-2-bromo-1-(2-fluoro-4-(tetrahydrofuran-2-yl)phenyl)ethan-1-one as a white solid.

Yield 240 mg (68% over two steps). ¹H NMR (400 MHz, CDCl₃) δ 7.94 (dd, J=6.8, 8.4 Hz, 1H), 7.28 (dd, J=2.0, 9.6 Hz, 1H), 7.26 (dd, J=2.0, 8.4 Hz, 1H), 4.90-4.82 (m, 1H), 4.54 (d, J=2.4 Hz, 2H), 3.84-3.68 (m, 2H), 1.91-1.80 (m, 2H), 1.79-1.69 (m, 2H).

m/z: [ESI] 303, 305 (M−H+18)⁻.

### Synthesis of ethyl (S)-2-(2-fluoro-4-(tetrahydrofuran-2-yl)phenyl)benzo[d]imidazo[2,1-b]thiazole-7-carboxylate (Intermediate 5, Scheme 76)

A solution of (S)-2-bromo-1-(2-fluoro-4-(tetrahydrofuran-2-yl)phenyl)ethan-1-one (240 mg, 0.836 mmol) and ethyl 2-aminobenzo[d]thiazole-6-carboxylate (186 mg, 0.837 mmol) in dioxane (5 mL) was stirred for 16 h at 120° C. under a nitrogen atmosphere. The resulting solution was allowed to cool down to room temperature and purified by reverse phase flash chromatography with the following conditions: Column: Spherical C18, 20-40 um, 120 g; Mobile Phase A: water (10 mM NH₄HCO₃); Mobile Phase B: acetonitrile; Flow rate: 80 mL/min; Gradient: 70% B-90% B in 20 min; Detector: 254 nm. The fractions containing desired product were collected at 88% B and concentrated under reduced pressure to afford ethyl (S)-2-(2-fluoro-4-(tetrahydrofuran-2-yl)phenyl)benzo[d]imidazo[2,1-b]thiazole-7-carboxylate as a white solid.

Yield: 120 mg (35%). ¹H NMR (400 MHz, CDCl₃) δ 8.49 (d, J=1.6 Hz, 1H), 8.26-8.19 (m, 3H), 7.75 (d, J=8.4 Hz, 1H), 7.25-7.18 (m, 2H), 4.96 (t, J=7.2 Hz, 1H), 4.46 (q, J=7.2 Hz, 2H), 4.18-4.10 (m, 1H), 4.03-3.94 (m, 1H), 2.45-2.33 (m, 1H), 2.10-2.00 (m, 2H), 1.91-1.78 (m, 1H), 1.47 (t, J=7.2 Hz, 3H). m/z: [ESI⁺] 411 (M+H)⁺.

### Synthesis of (S)-2-(2-fluoro-4-(tetrahydrofuran-2-yl)phenyl)benzo[d]imidazo[2,1-b]thiazole-7-carboxylic acid (Intermediate C1, Scheme 76)

To a stirred solution of ethyl (S)-2-(2-fluoro-4-(tetrahydrofuran-2-yl)phenyl)benzo[d]imidazo[2,1-b]thiazole-7-carboxylate (120 mg, 0.292 mmol) in TETRAHYDROFURAN (4 mL) and MeOH (4 mL) were added water (1 mL) and lithium hydroxide monohydrate (35 mg, 0.834 mmol) at room temperature. The reaction mixture was stirred for 2 h at room temperature. The resulting mixture was acidified to pH 5 with 1 N hydrochloride. The precipitated solids were collected by filtration and dried in the air to afford (S)-2-(2-fluoro-4-(tetrahydrofuran-2-yl)phenyl)benzo[d]imidazo[2,1-b]thiazole-7-carboxylic acid as a white solid.

Yield: 110 mg (98%). m/z: [ESI⁺] 383 (M+H)⁺. ¹HNMR not be ran.

### Synthesis of (R)-2-(2-fluoro-4-(tetrahydrofuran-2-yl)phenyl)benzo[d]imidazo[2,1-b]thiazole-7-carboxylic acid (Intermediate Dl, Scheme 77)

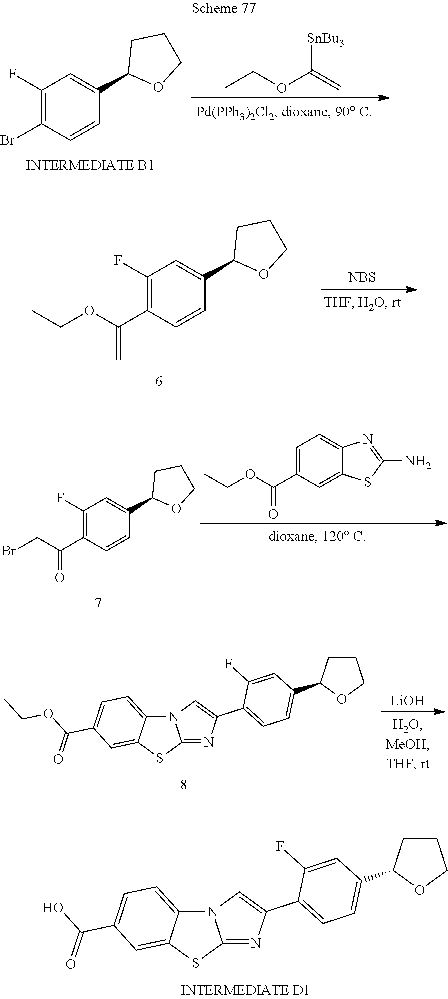

### Synthesis of (R)-2-(4-(1-ethoxyvinyl)-3-fluorophenyl)tetrahydrofuran (Intermediate 6, Scheme 77)

Compound (R)-2-(4-(1-ethoxyvinyl)-3-fluorophenyl)tetrahydrofuran was prepared from (R)-2-(4-bromo-3-fluorophenyl)tetrahydrofuran (350 mg, 1.428 mmol) and tributyl(1-ethoxyethenyl)stannane (774 mg, 2.143 mmol) following a similar procedure to that described for the synthesis of (S)-2-(4-(1-ethoxyvinyl)-3-fluorophenyl)tetrahydrofuran, and was isolated as a brown oil, which was unstable and was used in next step directly without further purification.

Yield 350 mg (crude). m/z: [ESI⁺] 255 (M+H+18)⁺. ¹HNMR not be ran.

### Synthesis of (R)-2-bromo-1-(2-fluoro-4-(tetrahydrofuran-2-yl)phenyl)ethan-1-one (Intermediate 7, Scheme 77)

Compound (R)-2-bromo-1-(2-fluoro-4-(tetrahydrofuran-2-yl)phenyl)ethan-1-one was prepared from (R)-2-(4-(1-ethoxyvinyl)-3-fluorophenyl)tetrahydrofuran (350 mg, 1.481 mmol) and 1-bromopyrrolidine-2,5-dione (264 mg, 1.483 mmol) following a similar procedure to that described for the synthesis of (S)-2-bromo-1-(2-fluoro-4-(tetrahydrofuran-2-yl)phenyl)ethan-1-one, and was isolated as a white solid.

Yield 280 mg (68% over two steps). ¹H NMR (400 MHz, CDCl₃) δ 7.94 (dd, J=6.8, 8.4 Hz, 1H), 7.28 (dd, J=2.0, 9.6 Hz, 1H), 7.26 (dd, J=2.0, 8.4 Hz, 1H), 4.90-4.82 (m, 1H), 4.54 (d, J=2.4 Hz, 2H), 3.84-3.68 (m, 2H), 1.91-1.80 (m, 2H), 1.79-1.69 (m, 2H).

m/z: [ESI⁻] 303, 305 (M−H+18)⁻.

### Synthesis of ethyl (R)-2-(2-fluoro-4-(tetrahydrofuran-2-yl)phenyl)benzo[d]imidazo[2,1-b]thiazole-7-carboxylate (Intermediate 8, Scheme 77)

Compound ethyl (R)-2-(2-fluoro-4-(tetrahydrofuran-2-yl)phenyl)benzo[d]imidazo[2,1-b]thiazole-7-carboxylate was prepared from (R)-2-bromo-1-(2-fluoro-4-(tetrahydrofuran-2-yl)phenyl)ethan-1-one (280 mg, 0.975 mmol) and ethyl 2-aminobenzo[d]thiazole-6-carboxylate (217 mg, 0.976 mmol) following a similar procedure to that described for the synthesis of ethyl (S)-2-(2-fluoro-4-(tetrahydrofuran-2-yl)phenyl)benzo[d]imidazo[2,1-b]thiazole-7-carboxylate, and was isolated as a white solid.

Yield: 110 mg (27%). ¹H NMR (400 MHz, CDCl₃) δ 8.49 (d, J=1.6 Hz, 1H), 8.26-8.19 (m, 3H), 7.75 (d, J=8.4 Hz, 1H), 7.25-7.18 (m, 2H), 4.96 (t, J=7.2 Hz, 1H), 4.46 (q, J=7.2 Hz, 2H), 4.18-4.10 (m, 1H), 4.03-3.94 (m, 1H), 2.45-2.33 (m, 1H), 2.10-2.00 (m, 2H), 1.91-1.78 (m, 1H), 1.47 (t, J=7.2 Hz, 3H). m/z: [ESI⁺] 411 (M+H)⁺.

### Synthesis of (R)-2-(2-fluoro-4-(tetrahydrofuran-2-yl)phenyl)benzo[d]imidazo[2,1-b]thiazole-7-carboxylic acid (Intermediate Dl, Scheme 77)

Compound (R)-2-(2-fluoro-4-(tetrahydrofuran-2-yl)phenyl)benzo[d]imidazo[2,1-b]thiazole-7-carboxylic acid was prepared from ethyl (R)-2-(2-fluoro-4-(tetrahydrofuran-2-yl)phenyl)benzo[d]imidazo[2,1-b]thiazole-7-carboxylate (110 mg, 0.268 mmol) following a similar procedure to that described for the synthesis of (S)-2-(2-fluoro-4-(tetrahydrofuran-2-yl)phenyl)benzo[d]imidazo[2,1-b]thiazole-7-carboxylic acid, and was isolated as a white solid.

Yield: 100 mg (98%). m/z: [ESI⁺] 383 (M+H)⁺. ¹HNMR not be ran.

### Synthesis of (S)-2-(2-fluoro-4-(5-oxopyrrolidin-2-yl)phenyl)benzo[d]imidazo[2,1-b]thiazole-7-carboxylic acid (Intermediate G1, Scheme 78)

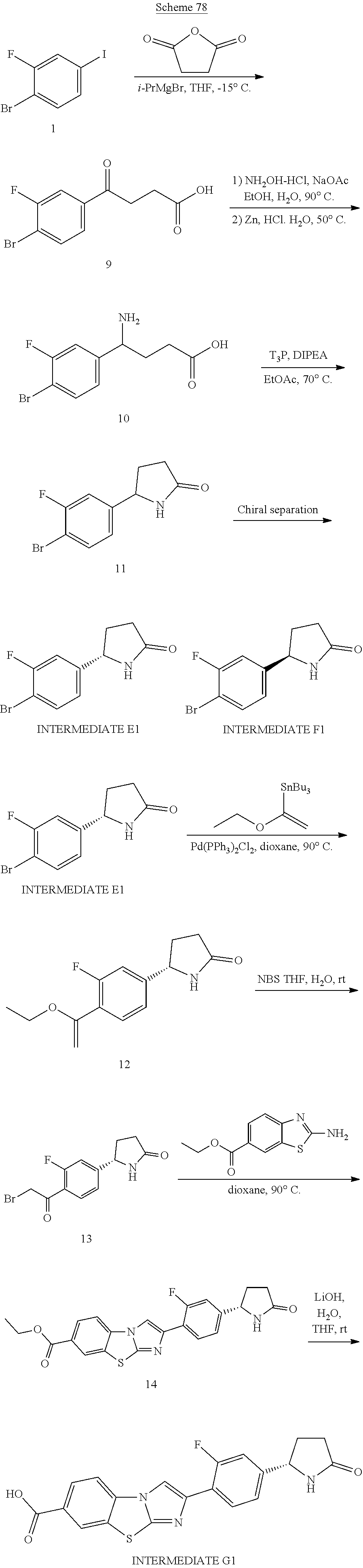

### Synthesis of 4-(4-bromo-3-fluorophenyl)-4-oxobutanoic acid (Intermediate 9, Scheme 78)

To a stirred solution of 1-bromo-2-fluoro-4-iodobenzene (50.00 g, 166.17 mmol) in tetrahydrofuran (240 mL) was added isopropylmagnesium bromide (1 N in tetrahydrofuran, 250 mL, 250.00 mmol) dropwise at −15° C. under a nitrogen atmosphere. The reaction solution was stirred for 30 min at −15° C. under a nitrogen atmosphere. The above solution was added dropwise to a solution of succinic anhydride (20.00 g, 199.85 mmol) in tetrahydrofuran (200 mL) at −15° C. under a nitrogen atmosphere. The resulting solution was stirred for additional 1 h at room temperature. The reaction was quenched with sat. ammonium chloride (aq.) (500 mL) at room temperature. The resulting mixture was acidified to pH 6 with 1 N hydrochloride and extracted with ethyl acetate (3×1000 mL). The combined organic layers were washed with brine (3×200 mL) and dried over anhydrous sodium sulfate. After filtration, the filtrate was concentrated under reduced pressure. The residue was purified by reverse phase column chromatography with the following conditions: Column: C18 Column 330 g; Mobile Phase A: water (0.05% v/v, FA), Mobile Phase B: acetonitrile; Flow rate: 90 mL/min; Gradient: 23% B to 43% B in 20 min; Wave Length: 254/200 nm. The fractions containing the desired product were collected at 37% B and concentrated under reduced pressure to afford 4-(4-bromo-3-fluorophenyl)-4-oxobutanoic acid as a white solid.

Yield: 6.40 g (14%). ¹H NMR (400 MHz, DMSO) δ 12.18 (br s, 1H), 7.94-7.87 (m, 2H), 7.79-7.74 (m, 1H), 3.26 (t, J=6.0 Hz, 2H), 2.58 (t, J=6.0 Hz, 2H). m/z: [ESI⁻] 273, 275 (M−H)⁻.

### Synthesis of 5-(4-bromo-3-fluorophenyl)pyrrolidin-2-one (Intermediate 11, Scheme 78)

To a stirred solution of hydroxylamine hydrochloride (1.94 g, 27.92 mmol) and sodium acetate (2.86 g, 34.86 mmol) in water (60 mL) was added a solution of 4-(4-bromo-3-fluorophenyl)-4-oxobutanoic acid (6.40 g, 23.27 mmol) in ethanol (50 mL) dropwise at room temperature. The reaction mixture was stirred for 16 h at 90° C. To the above solution were added hydrochloride (12 N, 4 mL) dropwise and zinc powder (7.40 g, 113.17 mmol) at room temperature. The resulting mixture was stirred for additional 2 h at 50° C. The mixture was allowed to cool down to room temperature and filtered. The filter cake was washed with ethanol (3×10 mL). The combined filtrates were concentrated under reduced pressure to afford 6.40 g crude 4-amino-4-(4-bromo-3-fluorophenyl)butanoic acid as light pink oil. This crude acid was dissolved in ethyl acetate (40 mL). To it were added N-ethyl-N-isopropylpropan-2-amine (41 mL, 235.38 mmol) and propanephosphonic acid cyclic anhydride (50% w/w in ethyl acetate, 40.00 g, 62.86 mmol) dropwise at room temperature. The reaction solution was stirred for overnight at 70° C. The resulting mixture was allowed to cool down to room temperature and concentrated under vacuum. The residue was purified by silica gel column chromatography, eluted with 1%-5% methanol in dichloromethane to afford 5-(4-bromo-3-fluorophenyl)pyrrolidin-2-one as a white solid.

Yield 3.00 g (50%). ¹H NMR (400 MHz, DMSO) δ 8.12 (br s, 1H), 7.70 (dd, J=8.0, 8.4 Hz, 1H), 7.31 (dd, J=2.0, 10.0 Hz, 1H), 7.12 (dd, J=2.0, 8.4 Hz, 1H), 4.74-4.64 (m, 1H), 2.49-2.42 (m, 1H), 2.27-2.20 (m, 2H), 1.80-1.70 (m, 1H). m/z: [ESI⁺] 258, 260 (M+H)⁺.

### Synthesis of (S)-5-(4-bromo-3-fluorophenyl)pyrrolidin-2-one and (R)-5-(4-bromo-3-fluorophenyl)pyrrolidin-2-one (Intermediates E1 and F1, Scheme 78)

5-(4-bromo-3-fluorophenyl)pyrrolidin-2-one (3.00 g, 11.62 mmol) was separated by Prep-Chiral-HPLC with the following conditions: Column: CHIRALPAK IG, 5×25 cm, 10 um; Mobile Phase A: CO₂, Mobile Phase B: Methanol (0.1% 2 N NH₃-Methanol); Flow rate: 200 mL/min; Gradient: isocratic 20% B; Column Temperature(° C.): 35; Back Pressure (bar): 100; Wave Length: 220 nm; RT1 (min): 12.53; RT2 (min): 16; Sample Solvent: Methanol:Dichloromethane=2:1. The faster eluting peak was collected and concentrated under reduced pressure to afford (R)-5-(4-bromo-3-fluorophenyl)pyrrolidin-2-one as a pink solid.

Yield 1.30 g (43%). ¹H NMR (400 MHz, CDCl₃) δ 7.57 (dd, J=7.6, 8.4 Hz, 1H), 7.10 (dd, J=2.0, 9.2 Hz, 1H), 7.01 (dd, J=2.0, 8.4 Hz, 1H), 6.56 (br s, 1H), 4.80-4.70 (m, 1H), 2.67-2.55 (m, 1H), 2.53-2.36 (m, 2H), 2.02-1.88 (m, 1H). m/z: [ESI⁺] 258, 260 (M+H)⁺.

The slower peak was collected and concentrated under reduced pressure to afford (S)-5-(4-bromo-3-fluorophenyl)pyrrolidin-2-one as a pink solid.

Yield 1.40 g (47%). ¹H NMR (400 MHz, CDCl₃) δ 7.57 (dd, J=7.6, 8.4 Hz, 1H), 7.10 (dd, J=2.0, 9.2 Hz, 1H), 7.01 (dd, J=2.0, 8.4 Hz, 1H), 6.56 (br s, 1H), 4.80-4.70 (m, 1H), 2.67-2.55 (m, 1H), 2.53-2.36 (m, 2H), 2.02-1.88 (m, 1H). m/z: [ESI⁺] 258, 260 (M+H)⁺.

### Synthesis of (S)-5-(4-(1-ethoxyvinyl)-3-fluorophenyl)pyrrolidin-2-one (Intermediate 12, Scheme 78)

Compound (S)-5-(4-(1-ethoxyvinyl)-3-fluorophenyl)pyrrolidin-2-one was prepared from (S)-5-(4-bromo-3-fluorophenyl)pyrrolidin-2-one (0.60 g, 2.33 mmol) and tributyl(1-ethoxyethenyl)stannane (1.26 g, 3.49 mmol) following a similar procedure to that described for the synthesis of (S)-2-(4-(1-ethoxyvinyl)-3-fluorophenyl)tetrahydrofuran, and was isolated as a brown oil, which was unstable and was used in next step directly without further purification.

Yield 0.55 g (crude). m/z: [ESI⁺] 250 (M+H)⁺.

### Synthesis of (S)-5-(4-(2-bromoacetyl)-3-fluorophenyl)pyrrolidin-2-one (Intermediate 13, Scheme 78)

Compound (S)-5-(4-(2-bromoacetyl)-3-fluorophenyl)pyrrolidin-2-one was prepared from (S)-5-(4-(1-ethoxyvinyl)-3-fluorophenyl)pyrrolidin-2-one (550 mg, 2.206 mmol) and 1-bromopyrrolidine-2,5-dione (982 mg, 5.517 mmol) following a similar procedure to that described for the synthesis of (S)-2-bromo-1-(2-fluoro-4-(tetrahydrofuran-2-yl)phenyl)ethan-1-one, and was isolated as a white solid.

Yield 440 mg (63% over two steps). ¹H NMR (400 MHz, CDCl₃) δ ¹H NMR (400 MHz, CDCl₃) δ 7.98 (dd, J=7.6, 8.4 Hz, 1H), 7.24 (dd, J=2.0, 9.2 Hz, 1H), 7.16 (dd, J=2.0, 8.4 Hz, 1H), 6.36 (br s, 1H), 4.89-4.79 (m, 1H), 4.52 (d, J=2.4 Hz, 2H), 2.73-2.59 (m, 1H), 2.57-2.38 (m, 2H), 2.05-1.92 (m, 1H). m/z: [ESI⁺] 300, 302 (M+H)⁺.

Synthesis of ethyl (S)-2-(2-fluoro-4-(5-oxopyrrolidin-2-yl)phenyl)benzo[d]imidazo[2,1-b]thiazole-7-carboxylate (Intermediate 14, Scheme 78)

Compound ethyl (S)-2-(2-fluoro-4-(5-oxopyrrolidin-2-yl)phenyl)benzo[d]imidazo[2,1-b]thiazole-7-carboxylate was prepared from (S)-5-(4-(2-bromoacetyl)-3-fluorophenyl)pyrrolidin-2-one (440 mg, 1.466 mmol) and ethyl 2-aminobenzo[d]thiazole-6-carboxylate (326 mg, 1.467 mmol) following a similar procedure to that described for the synthesis of ethyl (S)-2-(2-fluoro-4-(tetrahydrofuran-2-yl)phenyl)benzo[d]imidazo[2,1-b]thiazole-7-carboxylate, except that reacted at 90° C. and was isolated as a white solid.

Yield: 190 mg (31%). ¹H NMR (400 MHz, DMSO) δ 8.73 (d, J=3.6 Hz, 1H), 8.69 (d, J=1.6 Hz, 1H), 8.29 (d, J=8.4 Hz, 1H), 8.17-8.09 (m, 3H), 7.27 (dd, J=1.6, 6.8 Hz, 1H), 7.25 (d, J=2.0 Hz, 1H), 4.79-4.66 (m, 1H), 4.36 (q, J=7.2 Hz, 2H), 2.57-2.51 (m, 1H), 2.31-2.22 (m, 2H), 1.89-1.74 (m, 1H), 1.36 (t, J=7.2 Hz, 3H). m/z: [ESI⁺] 424 (M+H)⁺.

### Synthesis of (S)-2-(2-fluoro-4-(5-oxopyrrolidin-2-yl)phenyl)benzo[d]imidazo[2,1-b]thiazole-7-carboxylic acid (Intermediate G1, Scheme 78)

Compound (S)-2-(2-fluoro-4-(5-oxopyrrolidin-2-yl)phenyl)benzo[d]imidazo[2,1-b]thiazole-7-carboxylic acid was prepared from ethyl (S)-2-(2-fluoro-4-(5-oxopyrrolidin-2-yl)phenyl)benzo[d]imidazo[2,1-b]thiazole-7-carboxylate (190 mg, 0.449 mmol) following a similar procedure to that described for the synthesis of (S)-2-(2-fluoro-4-(tetrahydrofuran-2-yl)phenyl)benzo[d]imidazo[2,1-b]thiazole-7-carboxylic acid, and was isolated as a white solid.

Yield: 140 mg (79%). ¹H NMR (400 MHz, DMSO) δ 13.23 (br s, 1H), 8.74 (dd, J=2.0, 4.0 Hz, 1H), 8.67 (d, J=2.0 Hz, 1H), 8.29 (dd, J=2.0, 8.4 Hz, 1H), 8.20-8.06 (m, 3H), 7.29-7.22 (m, 2H), 4.78-4.68 (m, 1H), 2.57-2.51 (m, 1H), 2.31-2.22 (m, 2H), 1.89-1.72 (m, 1H). m/z: [ESI⁺] 396 (M+H)⁺.

### Synthesis of (R)-2-(2-fluoro-4-(5-oxopyrrolidin-2-yl)phenyl)benzo[d]imidazo[2,1-b]thiazole-7-carboxylic acid (Intermediate H1, Scheme 79)

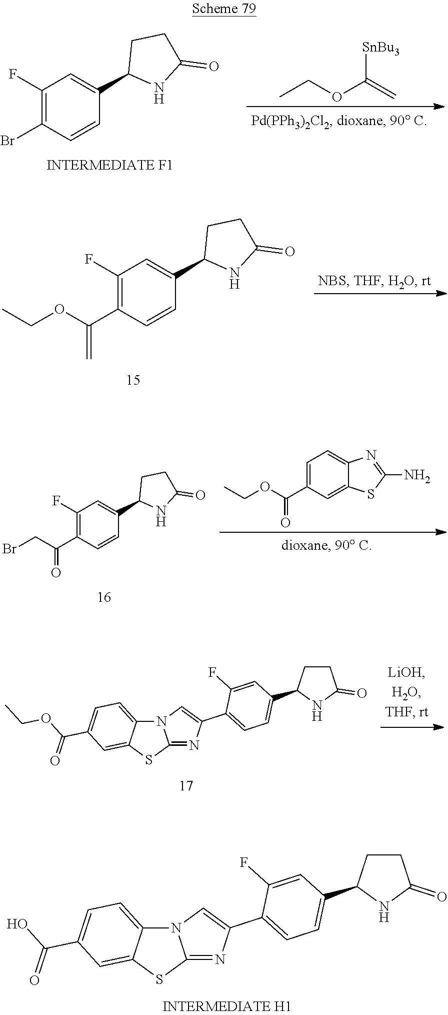

### Synthesis of (R)-5-(4-(1-ethoxyvinyl)-3-fluorophenyl)pyrrolidin-2-one (Intermediate 15, Scheme 79)

Compound (R)-5-(4-(1-ethoxyvinyl)-3-fluorophenyl)pyrrolidin-2-one was prepared from (R)-5-(4-bromo-3-fluorophenyl)pyrrolidin-2-one (0.80 g, 3.10 mmol) and tributyl(1-ethoxyethenyl)stannane (1.68 g, 4.65 mmol) following a similar procedure to that described for the synthesis of (S)-2-(4-(1-ethoxyvinyl)-3-fluorophenyl)tetrahydrofuran, and was isolated as a brown oil, which was unstable and was used in next step directly without further purification.

Yield 0.75 g (crude). m/z: [ESI⁺] 250 (M+H)⁺.

### Synthesis of (R)-5-(4-(2-bromoacetyl)-3-fluorophenyl)pyrrolidin-2-one (Intermediate 16, Scheme 79)

Compound (R)-5-(4-(2-bromoacetyl)-3-fluorophenyl)pyrrolidin-2-one was prepared from (R)-5-(4-(1-ethoxyvinyl)-3-fluorophenyl)pyrrolidin-2-one (0.75 g, 3.01 mmol) and 1-bromopyrrolidine-2,5-dione (1.34 g, 7.52 mmol) following a similar procedure to that described for the synthesis of (S)-2-bromo-1-(2-fluoro-4-(tetrahydrofuran-2-yl)phenyl)ethan-1-one, and was isolated as a white solid.

Yield 0.72 g (83% over two steps). ¹H NMR (400 MHz, CDCl₃) δ 7.98 (dd, J=7.6, 8.4 Hz, 1H), 7.24 (dd, J=2.0, 9.2 Hz, 1H), 7.16 (dd, J=2.0, 8.4 Hz, 1H), 6.36 (br s, 1H), 4.89-4.79 (m, 1H), 4.52 (d, J=2.4 Hz, 2H), 2.73-2.59 (m, 1H), 2.57-2.38 (m, 2H), 2.05-1.92 (m, 1H). m/z: [ESI⁺] 300, 302 (M+H)⁺.

### Synthesis of ethyl (R)-2-(2-fluoro-4-(5-oxopyrrolidin-2-yl)phenyl)benzo[d]imidazo[2,1-b]thiazole-7-carboxylate (Intermediate 17, Scheme 79)

Compound ethyl (R)-2-(2-fluoro-4-(5-oxopyrrolidin-2-yl)phenyl)benzo[d]imidazo[2,1-b]thiazole-7-carboxylate was prepared from (R)-5-(4-(2-bromoacetyl)-3-fluorophenyl)pyrrolidin-2-one (720 mg, 2.399 mmol) and ethyl 2-aminobenzo[d]thiazole-6-carboxylate (533 mg, 2.398 mmol) following a similar procedure to that described for the synthesis of ethyl (S)-2-(2-fluoro-4-(tetrahydrofuran-2-yl)phenyl)benzo[d]imidazo[2,1-b]thiazole-7-carboxylate, except that reacted at 90° C. and was isolated as a white solid.

Yield: 142 mg (14%). ¹H NMR (400 MHz, DMSO) δ 8.73 (d, J=3.6 Hz, 1H), 8.69 (d, J=1.6 Hz, 1H), 8.29 (d, J=8.4 Hz, 1H), 8.17-8.09 (m, 3H), 7.27 (dd, J=1.6, 6.8 Hz, 1H), 7.25 (d, J=2.0 Hz, 1H), 4.79-4.66 (m, 1H), 4.36 (q, J=7.2 Hz, 2H), 2.57-2.51 (m, 1H), 2.31-2.22 (m, 2H), 1.89-1.74 (m, 1H), 1.36 (t, J=7.2 Hz, 3H). m/z: [ESI⁺] 424 (M+H)⁺.

### Synthesis of (R)-2-(2-fluoro-4-(5-oxopyrrolidin-2-yl)phenyl)benzo[d]imidazo[2,1-b]thiazole-7-carboxylic acid (Intermediate H1, Scheme 79)

Compound (R)-2-(2-fluoro-4-(5-oxopyrrolidin-2-yl)phenyl)benzo[d]imidazo[2,1-b]thiazole-7-carboxylic acid was prepared from ethyl (R)-2-(2-fluoro-4-(5-oxopyrrolidin-2-yl)phenyl)benzo[d]imidazo[2,1-b]thiazole-7-carboxylate (142 mg, 0.335 mmol) following a similar procedure to that described for the synthesis of (S)-2-(2-fluoro-4-(tetrahydrofuran-2-yl)phenyl)benzo[d]imidazo[2,1-b]thiazole-7-carboxylic acid, and was isolated as a white solid.

Yield: 100 mg (75%). ¹H NMR (400 MHz, DMSO) δ 13.23 (br s, 1H), 8.74 (dd, J=2.0, 4.0 Hz, 1H), 8.67 (d, J=2.0 Hz, 1H), 8.29 (dd, J=2.0, 8.4 Hz, 1H), 8.20-8.06 (m, 3H), 7.29-7.22 (m, 2H), 4.78-4.68 (m, 1H), 2.57-2.51 (m, 1H), 2.31-2.22 (m, 2H), 1.89-1.72 (m, 1H). m/z: [ESI⁺] 396 (M+H)⁺.

### Example 5

### Synthetic Details for Various Compounds of the Invention (Schemes 80-93)

### N-((1s,3s)-3-(methylamino)cyclobutyl)-2-(m-tolyl)benzo[d]imidazo[2,1-b]thiazole-7-carboxamide formate (Compound 157); and

### N-((1r,3r)-3-(methylamino)cyclobutyl)-2-(m-tolyl)benzo[d]imidazo[2,1-b]thiazole-7-carboxamide formate (Compound 158)

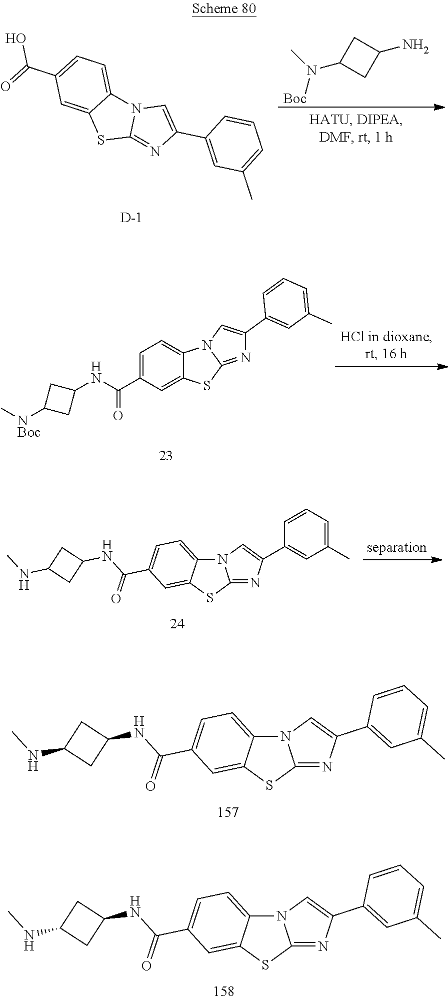

### Step 1: Tert-butyl methyl(3-(2-(m-tolyl)benzo[d]imidazo[2,1-b]thiazole-7-carboxamido)cyclobutyl)carbamate

To a stirred solution of 2-(m-tolyl)benzo[d]imidazo[2,1-b]thiazole-7-carboxylic acid (200 mg, 0.649 mmol) in DMF (2 mL) were added HATU (321 mg, 0.844 mmol), tert-butyl (3-aminocyclobutyl)(methyl)carbamate (156 mg, 0.779 mmol) and DIPEA (251 mg, 1.942 mmol) at room temperature under a nitrogen atmosphere. The resulting mixture was stirred for 1 h at room temperature under a nitrogen atmosphere. The resulting mixture was purified by reverse phase flash chromatography with the following conditions: Column: Spherical C18, 20-40 μm, 330 g; Mobile Phase A: water (plus 10 mM NH₄HCO₃); Mobile Phase B: ACN; Flow rate: 80 mL/min; Gradient: 80% B-95% B in 20 min; Detector: UV 220/254 nm. The fractions containing desired product were collected and concentrated under reduced pressure to afford tert-butyl methyl(3-(2-(m-tolyl)benzo[d]imidazo[2,1-b]thiazole-7-carboxamido)cyclobutyl)carbamate as an off-white solid.

Yield 250 mg (79%). ¹H NMR (400 MHz, DMSO) δ 8.90 (d, J=6.4 Hz, 0.75H), 8.82 (s, 1H), 8.73 (d, J=6.4 Hz, 0.25H), 8.52 (d, J=1.6 Hz, 0.75H), 8.49 (d, J=1.6 Hz, 0.25H), 8.12-8.00 (m, 2H), 7.72 (s, 1H), 7.67 (d, J=7.6 Hz, 1H), 7.34 (dd, J=1.6, 7.6 Hz, 1H), 7.13 (d, J=7.6 Hz, 1H), 4.91-4.72 (m, 0.75H), 4.35-4.25 (m, 0.75H), 4.15 (q, J=8.0 Hz, 0.25H), 3.32-3.30 (m, 0.25H), 2.82 (s, 2.25H), 2.81 (s, 0.75H), 2.60-2.52 (m, 2H), 2.38 (s, 3H), 2.27 (d, J=12.8 Hz, 2H), 1.41 (s, 9H). (A mixture of trans/cis isomers with a ratio of 3:1). m/z: [ESI⁺] 491 (M+H)⁺.

### Analytical Data for Compounds Synthesized Based on the Methods Described Above

The following compounds below were synthesized according to the described procedure above. The purifications by reverse phase chromatography with the addition of NH₄HCO₃ produced the parent compound while with the addition of formic acid, produced the compound as formate form.

Ethyl 2-(4-(dimethylcarbamoyl)phenyl)benzo[d]imidazo[2,1-b]thiazole-7-carboxylate: Starting from 4-(7-(ethoxycarbonyl)benzo[d]imidazo[2,1-b]thiazol-2-yl)benzoic acid (0.50 g, 1.36 mmol). Yield 0.50 g (93%), as an off-white solid. ¹H NMR (400 MHz, DMSO) δ 8.94 (s, 1H), 8.72 (d, J=1.6 Hz, 1H), 8.17 (dd, J=1.6, 8.4 Hz, 1H), 8.10 (d, J=8.4 Hz, 1H), 7.93 (d, J=8.4 Hz, 2H), 7.50 (d, J=8.4 Hz, 2H), 4.37 (q, J=7.2 Hz, 2H), 2.99 (s, 3H), 2.87 (s, 3H), 1.37 (t, J=7.2 Hz, 3H). m/z: [ESI⁺] 394 (M+H)⁺.

Ethyl 2-(4-(methylcarbamoyl)phenyl)benzo[d]imidazo[2,1-b]thiazole-7-carboxylate: Starting from 4-(7-(ethoxycarbonyl)benzo[d]imidazo[2,1-b]thiazol-2-yl)benzoic acid (12.00 g, 32.75 mmol). Yield 10.00 g (80%), as a white solid. ¹H NMR (400 MHz, DMSO) δ 8.98 (s, 1H), 8.73 (d, J=1.6 Hz, 1H), 8.46 (d, J=4.2 Hz, 1H), 8.17 (dd, J=1.6, 8.4, Hz, 1H), 8.09 (d, J=8.4 Hz, 1H), 8.09 (q, J=8.6 Hz, 2H), 7.94 (q, J=8.6 Hz, 2H), 4.38 (q, J=7.2 Hz, 2H), 2.81 (d, J=4.2 Hz, 3H), 1.37 (t, J=7.2 Hz, 3H). m/z: [ESI⁺] 380 (M+H)⁺.

2-(4-Bromophenyl)-N-(3-(diethylamino)propyl)benzo[d]imidazo[2,1-b]thiazole-7-carboxamide: Starting from 2-(4-bromophenyl)benzo[d]imidazo[2,1-b]thiazole-7-carboxylic acid formate (5.80 g, 15.54 mmol). Yield 3.20 g (42%), as a brown solid. ¹H NMR (400 MHz, DMSO) δ 8.88 (s, 1H), 8.67 (t, J=5.4 Hz, 1H), 8.49 (d, J=1.6 Hz, 1H), 8.21 (s, 1H), 8.05 (d, J=8.4 Hz, 1H), 8.02 (dd, J=1.6, 8.4 Hz, 1H), 7.83 (d, J=8.6 Hz, 2H), 7.65 (d, J=8.6 Hz, 2H), 3.34 (q, J=6.6 Hz, 2H), 2.66-2.55 (m, 6H), 1.73-1.71 (m, 2H), 1.01 (t, J=7.2 Hz, 6H). m/z: [ESI⁺] 485, 487 (M+H)⁺.

2-(3-Bromophenyl)-N-(3-(diethylamino)propyl)benzo[d]imidazo[2,1-b]thiazole-7-carboxamide: Starting from 2-(3-bromophenyl)benzo[d]imidazo[2,1-b]thiazole-7-carboxylic acid (7.00 g, 18.76 mmol). Yield 6.00 g (66%), as a brown solid. ¹H NMR (400 MHz, DMSO) δ 8.92 (s, 1H), 8.64 (dd, J=5.4 Hz, 1H), 8.48 (d, J=1.6 Hz, 1H), 8.05 (dd, J=1.6, 2.0 Hz, 1H), 8.04-8.00 (m, 2H), 7.87 (dd, J=1.4, 7.6 Hz, 1H), 7.49 (dd, J=1.4, 8.0 Hz, 1H), 7.41 (dd, J=1.6, 7.8 Hz, 1H), 3.35-3.27 (m, 2H), 2.52-2.41 (m, 6H), 1.67 (p, J=7.2 Hz, 2H), 0.95 (t, J=7.2 Hz, 6H). m/z: [ESI⁺] 485, 487 (M+H)⁺.

2-Bromo-N-(3-(diethylamino)propyl)benzo[d]imidazo[2,1-b]thiazole-7-carboxamide: Starting from 2-bromobenzo[d]imidazo[2,1-b]thiazole-7-carboxylic acid (20.00 g, 67.31 mmol). Yield 18.15 g (66%), as an off-white solid. ¹H NMR (400 MHz, DMSO) δ 8.63 (dd, J=5.4 Hz, 1H), 8.56 (s, 1H), 8.51 (d, J=1.6 Hz, 1H), 8.09 (d, J=8.4 Hz, 1H), 8.00 (dd, J=1.6, 8.4 Hz, 1H), 3.33-3.26 (m, 2H), 2.50-2.46 (m, 6H), 1.68-1.66 (m, 2H), 0.95 (t, J=7.2 Hz, 6H). m/z: [ESI⁺] 409, 411 (M+H)⁺.

Tert-butyl 4-(2-(m-tolyl)benzo[d]imidazo[2,1-b]thiazole-7-carboxamido)piperidine-1-carboxylate: Starting from 2-(m-tolyl)benzo[d]imidazo[2,1-b]thiazole-7-carboxylic acid (0.30 g, 0.97 mmol). Yield 0.41 g (87%), as an off-white solid. ¹H NMR (400 MHz, DMSO) δ 8.81 (s, 1H), 8.49 (d, J=1.6 Hz, 1H), 8.42 (d, J=7.6 Hz, 1H), 8.06-8.02 (m, 2H), 7.71 (s, 1H), 7.66 (d, J=7.8 Hz, 1H), 7.33 (dd, J=1.6, 7.6 Hz, 1H), 7.12 (d, J=7.6 Hz, 1H), 3.98 (m, 3H), 2.98-2.77 (m, 2H), 2.37 (s, 3H), 1.82 (d, J=12.4 Hz, 2H), 1.46-1.40 (m, 2H), 1.42 (s, 9H). m/z: [ESI⁺] 491 (M+H)⁺.

Tert-butyl 4-(2-(m-tolyl)benzo[d]imidazo[2,1-b]thiazole-7-carbonyl)piperazine-1-carboxylate: Starting from 2-(m-Tolyl)benzo[d]imidazo[2,1-b]thiazole-7-carboxylic acid (0.30 g, 0.97 mmol). Yield 0.42 g (91%), as an off-white solid. ¹H NMR (400 MHz, DMSO) δ 8.82 (s, 1H), 8.15 (d, J=1.6 Hz, 1H), 8.03 (d, J=8.2 Hz, 1H), 7.71 (s, 1H), 7.67 (d, J=7.8 Hz, 1H), 7.63 (dd, J=1.6, 8.2 Hz, 1H), 7.33 (dd, J=1.6, 7.6 Hz, 1H), 7.12 (d, J=7.6 Hz, 1H), 3.67-3.52 (m, 2H), 3.45-3.36 (m, 6H), 2.37 (s, 3H), 1.42 (s, 9H). m/z: [ESI⁺] 477 (M+H)⁺.

Tert-butyl 2-(2-(2-(m-tolyl)benzo[d]imidazo[2,1-b]thiazole-7-carboxamido)ethyl)pyrrolidine-1-carboxylate: Starting from 2-(m-tolyl)benzo[d]imidazo[2,1-b]thiazole-7-carboxylic acid (0.35 g, 1.14 mmol). Yield 0.45 g (78%), as a brown solid. ¹H NMR (400 MHz, DMSO) δ 8.55 (t, J=5.4 Hz, 1H), 8.39 (s, 1H), 8.09 (d, J=8.4 Hz, 1H), 8.02 (s, 1H), 7.77 (d, J=1.8 Hz, 1H), 7.68 (d, J=8.4 Hz, 2H), 7.34 (dd, J=1.6, 7.6 Hz, 1H), 7.16 (d, J=7.6 Hz, 1H), 4.11-4.03 (m, 1H), 3.97-3.87 (m, 1H), 3.39 (t, J=6.8 Hz, 2H), 3.15-3.06 (m, 1H), 2.45 (s, 3H), 2.06-1.91 (m, 1H), 1.87-1.76 (m, 1H), 1.77-1.55 (m, 4H), 1.54 (s, 9H). m/z: [ESI⁺] 505 (M+H)⁺.

Tert-butyl ethyl(3-(2-(m-tolyl)benzo[d]imidazo[2,1-b]thiazole-7-carboxamido)propyl)carbamate: Starting from 2-(m-tolyl)benzo[d]imidazo[2,1-b]thiazole-7-carboxylic acid (0.30 g, 0.97 mmol). Yield 0.40 g (84%), as an off-white solid. ¹H NMR (400 MHz, DMSO) δ 8.80 (s, 1H), 8.59 (t, J=5.4 Hz, 1H), 8.49 (d, J=1.6 Hz, 1H), 8.09-7.99 (m, 2H), 7.71 (d, J=1.8 Hz, 1H), 7.67 (d, J=7.8 Hz, 1H), 7.33 (dd, J=1.6, 7.6 Hz, 1H), 7.13 (dd, J=1.8, 7.6 Hz, 1H), 3.29 (q, J=6.8 Hz, 2H), 3.21-3.19 (m, 4H), 2.38 (s, 3H), 1.77-1.75 (m, 2H), 1.39 (s, 9H), 1.05 (t, J=7.2 Hz, 3H). m/z: [ESI⁺] 493 (M+H)⁺.

Tert-butyl 4-(3-(2-(4-(methylcarbamoyl)phenyl)benzo[d]imidazo[2,1-b]thiazole-7-carboxamido)propyl)piperazine-1-carboxylate: Starting from 2-(4-(methylcarbamoyl)phenyl)benzo[d]imidazo[2,1-b]thiazole-7-carboxylic acid (0.20 g, 0.57 mmol). Yield 0.20 g (61%), as a white solid. ¹H NMR (400 MHz, DMSO) δ 8.94 (s, 1H), 8.63 (t, J=5.6 Hz, 1H), 8.50 (d, J=1.6 Hz, 1H), 8.46 (q, J=4.4 Hz, 1H), 8.06 (d, J=8.4 Hz, 1H), 8.03 (dd, J=1.6, 8.4 Hz, 1H), 7.97-7.91 (m, 4H), 3.34-3.26 (m, 6H), 2.81 (d, J=4.4 Hz, 3H), 2.50-2.31 (m, 6H), 1.76-1.74 (m, 2H), 1.40 (s, 9H). m/z: [ESI⁺] 577 (M+H)⁺.

Tert-butyl (3-(2-(m-tolyl)benzo[d]imidazo[2,1-b]thiazole-7-carboxamido)propyl)(2,2,2-trifluoroethyl)carbamate: Starting from 2-(m-tolyl)benzo[d]imidazo[2,1-b]thiazole-7-carboxylic acid (0.32 g, 1.04 mmol). Yield 0.40 g (70%), as an off-white solid. ¹H NMR (400 MHz, DMSO) δ 8.81 (s, 1H), 8.62 (s, 1H), 8.49 (d, J=1.6 Hz, 1H), 8.06 (d, J=8.4 Hz, 1H), 8.03 (dd, J=1.6, 8.4 Hz, 1H), 7.71 (d, J=2.0 Hz, 1H), 7.67 (d, J=7.8 Hz, 1H), 7.33 (dd, J=1.6, 7.6 Hz, 1H), 7.16-7.09 (m, 1H), 4.06 (q, J=9.4 Hz, 2H), 3.32-3.25 (m, 4H), 2.38 (s, 3H), 1.85-1.76 (m, 2H), 1.40 (s, 9H). m/z: [ESI⁺] 547 (M+H)⁺.

Tert-butyl 4-((2-(m-tolyl)benzo[d]imidazo[2,1-b]thiazol-7-yl)carbamoyl)piperidine-1-carboxylate: Starting from 2-(m-tolyl)benzo[d]imidazo[2,1-b]thiazol-7-amine (0.13 g, 0.47 mmol). Yield 0.15 g (66%), as a white solid. ¹H NMR (400 MHz, DMSO) δ 10.23 (br s, 1H), 8.69 (s, 1H), 8.33 (d, J=2.0 Hz, 1H), 7.90 (d, J=8.6 Hz, 1H), 7.70 (d, J=1.8 Hz, 1H), 7.68-7.63 (m, 2H), 7.31 (dd, J=1.6, 7.6 Hz, 1H), 7.10 (d, J=7.6 Hz, 1H), 4.02 (d, J=12.9 Hz, 2H), 2.90-2.71 (m, 2H), 2.60-2.54 (m, 1H), 2.37 (s, 3H), 1.81 (dd, J=3.6, 13.6 Hz, 2H), 1.51 (dq, J=4.4, 12.4 Hz, 2H), 1.42 (s, 9H). m/z: [ESI⁺] 491 (M+H)⁺.

Tert-butyl 3-((2-(p-tolyl)benzo[d]imidazo[2,1-b]thiazole-7-carboxamido)methyl)azetidine-1-carboxylate: Starting from 2-(p-tolyl)benzo[d]imidazo[2,1-b]thiazole-7-carboxylic acid (0.50 g, 1.62 mmol). Yield 0.35 g (45%), as a white solid. ¹H NMR (400 MHz, DMSO) δ 8.75 (s, 1H), 8.74 (t, J=5.4 Hz, 1H), 8.48 (d, J=1.6 Hz, 1H), 8.06 (d, J=8.4 Hz, 1H), 8.02 (dd, J=1.6, 8.4 Hz, 1H) 7.77 (d, J=8.0 Hz, 2H), 7.26 (d, J=8.0 Hz, 2H), 3.97-3.87 (m, 2H), 3.70-3.60 (m, 2H), 3.50 (t, J=6.4 Hz, 2H), 2.80-2.72 (m, 1H), 2.34 (s, 3H), 1.37 (s, 9H). m/z: [ESI⁺] 477 (M+H)⁺.

Tert-butyl 3-((2-(p-tolyl)benzo[d]imidazo[2,1-b]thiazole-7-carboxamido)methyl)pyrrolidine-1-carboxylate: Starting from 2-(p-tolyl)benzo[d]imidazo[2,1-b]thiazole-7-carboxylic acid (0.50 g, 1.62 mmol). Yield 0.45 g (57%), as a white solid. ¹H NMR (400 MHz, DMSO) δ 8.76 (s, 1H), 8.69 (t, J=5.4 Hz, 1H), 8.49 (d, J=1.6 Hz, 1H), 8.08-8.01 (m, 2H), 7.77 (d, J=8.0 Hz, 2H), 7.26 (d, J=8.0 Hz, 2H), 3.39-3.30 (m, 4H), 3.28-3.18 (m, 1H), 3.03 (t, J=9.2 Hz, 1H), 2.50-2.46 (m, 1H), 2.34 (s, 3H), 2.00-1.88 (m, 1H), 1.71-1.59 (m, 1H), 1.40 (s, 9H). m/z: [ESI⁺]491 (M+H)⁺.

Tert-butyl (S)-(2-(2-(4-(methylcarbamoyl)phenyl)benzo[d]imidazo[2,1-b]thiazole-7-carboxamido)propyl)carbamate: Starting from 2-(4-(methylcarbamoyl)phenyl)benzo[d]imidazo[2,1-b]thiazole-7-carboxylic acid (0.15 g, 0.43 mmol). Yield 0.15 g (70%), as a white solid. ¹H NMR (400 MHz, DMSO) δ 8.94 (d, J=5.5 Hz, 1H), 8.51-8.41 (m, 2H), 8.27 (d, J=8.1 Hz, 1H), 8.10-8.00 (m, 2H), 7.98-7.88 (m, 4H), 6.97 (t, J=6.1 Hz, 1H), 4.12-3.99 (m, 1H), 3.09 (d, J=6.4 Hz, 1H), 2.94 (s, 1H), 2.81 (d, J=4.3 Hz, 3H), 1.37 (s, 9H), 1.13 (d, J=6.6 Hz, 3H). m/z: [ESI⁺] 508 (M+H)⁺.

Tert-butyl (1-((2-(4-(methylcarbamoyl)phenyl)benzo[d]imidazo[2,1-b]thiazole-7-carboxamido)methyl)cyclopropyl)carbamate: Starting from 2-(4-(methylcarbamoyl)phenyl)benzo[d]imidazo[2,1-b]thiazole-7-carboxylic acid (0.15 g, 0.43 mmol). Yield 0.15 g (67%), as a brown solid. ¹H NMR (400 MHz, DMSO) δ 8.95 (s, 1H), 8.55 (t, J=5.4 Hz, 1H), 8.50 (s, 1H), 8.45 (q, J=4.4 Hz, 1H), 8.11-8.01 (m, 2H), 7.95 (d, J=8.6 Hz, 2H), 7.92 (d, J=8.6 Hz, 2H), 7.24 (br s, 1H), 3.42 (d, J=5.4 Hz, 2H), 2.81 (d, J=4.4 Hz, 3H), 1.38 (s, 9H), 0.83-0.77 (m, 2H), 0.71-0.62 (m, 2H). m/z: [ESI⁺] 520 (M+H)⁺.

Tert-butyl (3R,4R)-3-hydroxy-4-((2-(4-(methylcarbamoyl)phenyl)benzo[d]imidazo[2,1-b]thiazole-7-carboxamido)methyl)piperidine-1-carboxylate: Starting from 2-(4-(methylcarbamoyl)phenyl)benzo[d]imidazo[2,1-b]thiazole-7-carboxylic acid (0.15 g, 0.43 mmol). Yield 0.15 g (63%), as an off-white solid. ¹H NMR (400 MHz, DMSO) δ 8.93 (s, 1H), 8.51 (t, J=5.4 Hz, 1H), 8.50 (d, J=1.4 Hz, 1H), 8.44 (q, J=4.8 Hz, 1H), 8.09-8.00 (m, 2H), 7.95 (d, J=8.6 Hz, 2H), 7.91 (d, J=8.6 Hz, 2H), 4.00 (d, J=12.4 Hz, 1H), 3.87 (d, J=13.2 Hz, 1H), 3.63 (dd, J=4.2, 13.2 Hz, 1H), 3.33-3.20 (m, 1H), 3.16 (dt, J=4.8, 9.8 Hz, 1H), 2.81 (s, 3H), 2.60-2.50 (m, 2H), 1.82-1.71 (m, 1H), 1.60 (dq, J=7.6, 11.6 Hz, 1H), 1.39 (s, 9H), 1.19-1.03 (m, 1H). m/z: [ESI⁺] 564 (M+H)⁺.

Tert-butyl (1R,5S,6s)-6-(2-(4-(methylcarbamoyl)phenyl)benzo[d]imidazo[2,1-b]thiazole-7-carboxamido)-3-azabicyclo[3.1.0]hexane-3-carboxylate: Starting from 2-(4-(methylcarbamoyl)phenyl)benzo[d]imidazo[2,1-b]thiazole-7-carboxylic acid (0.15 g, 0.43 mmol). Yield 0.10 g (44%), as a brown solid. ¹H NMR (400 MHz, DMSO) δ 8.92 (s, 1H), 8.68 (d, J=4.0 Hz, 1H), 8.49-8.42 (m, 2H), 8.05 (d, J=8.4 Hz, 1H), 8.00 (dd, J=1.6, 8.4 Hz, 1H), 7.94 (d, J=8.6 Hz, 2H), 7.91 (d, J=8.6 Hz, 2H), 3.55 (d, J=10.8 Hz, 2H), 3.38 (d, J=12.1 Hz, 2H), 2.80 (d, J=4.4 Hz, 3H), 2.60-2.50 (m, 1H), 1.82 (d, J=3.0 Hz, 2H), 1.40 (s, 9H). m/z: [ESI⁺] 532 (M+H)⁺.

Tert-butyl ((1-(2-(4-(methylcarbamoyl)phenyl)benzo[d]imidazo[2,1-b]thiazole-7-carboxamido)cyclobutyl)methyl)carbamate: Starting from 2-(4-(methylcarbamoyl)phenyl)benzo[d]imidazo[2,1-b]thiazole-7-carboxylic acid (0.10 g, 0.28 mmol). Yield 0.12 g (79%), as a white solid. ¹H NMR (400 MHz, DMSO) δ 8.95 (s, 1H), 8.49 (s, 1H), 8.46 (q, J=4.4 Hz, 1H), 8.28 (d, J=8.0 Hz, 1H), 8.06 (d, J=8.4 Hz, 1H), 8.03 (d, J=8.4 Hz, 1H), 7.95 (d, J=8.6 Hz, 2H), 7.92 (d, J=8.6 Hz, 2H), 6.97 (t, J=6.0 Hz, 1H), 3.32 (d, J=5.6 Hz, 2H), 3.12-3.10 (m, 2H), 2.81 (d, J=4.3 Hz, 3H), 2.60-2.50 (m, 4H), 1.37 (s, 9H). m/z: [ESI⁺] 534 (M+H)⁺.

Tert-butyl (1R,4R,5S)-5-(2-(4-(methylcarbamoyl)phenyl)benzo[d]imidazo[2,1-b]thiazole-7-carboxamido)-2-azabicyclo[2.1.1]hexane-2-carboxylate: Starting from 2-(4-(methylcarbamoyl)phenyl)benzo[d]imidazo[2,1-b]thiazole-7-carboxylic acid (0.15 g, 0.43 mmol). Yield 0.15 g (66%), as a white solid. ¹H NMR (400 MHz, DMSO) δ 8.92 (s, 1H), 8.47-8.44 (m, 2H), 8.34 (d, J=3.8 Hz, 1H), 8.05 (d, J=8.4 Hz, 1H), 7.99-7.89 (m, 5H), 4.40-4.30 (m, 1H), 3.80-3.72 (m, 1H), 3.58-3.49 (m, 1H), 3.21-3.12 (m, 1H), 2.81 (d, J=4.4 Hz, 3H), 2.55-2.50 (m, 2H), 1.72 (d, J=7.6 Hz, 1H), 1.26 (s, 9H). m/z: [ESI⁺] 532 (M+H)⁺.

Tert-butyl (S)-(4-(2-(4-(methylcarbamoyl)phenyl)benzo[d]imidazo[2,1-b]thiazole-7-carboxamido)butan-2-yl)carbamate: Starting from 2-(4-(methylcarbamoyl)phenyl)benzo[d]imidazo[2,1-b]thiazole-7-carboxylic acid (0.15 g, 0.43 mmol). Yield 0.15 g (67%), as a white solid. ¹H NMR (400 MHz, CDCl₃) δ 8.34 (s, 1H), 8.08 (s, 1H), 8.03 (d, J=8.4 Hz, 1H), 7.98-7.91 (m, 3H), 7.88-7.81 (m, 3H), 7.69 (d, J=8.4 Hz, 1H), 6.34 (d, J=5.4 Hz, 1H), 4.06-3.96 (m, 1H), 3.90-3.80 (m, 1H), 3.12-3.05 (m, 1H), 3.05 (d, J=4.6 Hz, 3H), 1.96-1.86 (m, 1H), 1.82-1.74 (m, 1H), 1.51 (s, 9H), 1.23 (d, J=6.8 Hz, 3H). m/z: [ESI⁺] 522 (M+H)⁺.

Tert-butyl (S)-3-(2-(4-(methylcarbamoyl)phenyl)benzo[d]imidazo[2,1-b]thiazole-7-carboxamido)pyrrolidine-1-carboxylate: Starting from 2-(4-(methylcarbamoyl)phenyl)benzo[d]imidazo[2,1-b]thiazole-7-carboxylic acid (0.10 g, 0.29 mmol).

Yield 0.11 g (74%), as a white solid. ¹H NMR (400 MHz, DMSO) δ 8.94 (s, 1H), 8.69 (d, J=6.4 Hz, 1H), 8.52 (s, 1H), 8.47-8.45 (m, 1H), 8.06 (s, 2H), 7.95 (d, J=8.6 Hz, 2H), 7.91 (d, J=8.6 Hz, 2H), 4.46 (p, J=6.0 Hz, 1H), 3.60-3.56 (m, 1H), 3.44 (td, J=7.2, 10.8 Hz, 1H), 3.24 (dt, J=4.6, 10.0 Hz, 1H), 2.90-2.80 (m, 1H), 2.81 (d, J=4.4 Hz, 3H), 2.13 (td, J=6.8, 13.4 Hz, 1H), 1.93 (pd, J=6.8, 13.4 Hz, 1H), 1.42 (s, 9H). m/z: [ESI⁺] 521 (M+H)⁺.

Tert-butyl (R)-2-((2-(4-(methylcarbamoyl)phenyl)benzo[d]imidazo[2,1-b]thiazole-7-carboxamido)methyl)pyrrolidine-1-carboxylate: Starting from 2-(4-(methylcarbamoyl)phenyl)benzo[d]imidazo[2,1-b]thiazole-7-carboxylic acid (0.15 g, 0.43 mmol). Yield 0.09 g (40%), as an off-white solid. ¹H NMR (400 MHz, CDCl₃) δ 8.76 (s, 1H), 8.33 (s, 1H), 8.08 (s, 1H), 8.07-8.02 (m, 1H), 7.94 (d, J=8.6 Hz, 2H), 7.84 (d, J=8.6 Hz, 2H), 7.68 (d, J=8.4 Hz, 1H), 6.25 (q, J=5.4 Hz, 1H), 4.29 (t, J=10.0 Hz, 1H), 3.63-3.57 (m, 1H), 3.48-3.38 (m, 3H), 3.07 (d, J=4.8 Hz, 3H), 2.15-2.10 (m, 1H), 2.03-1.92 (m, 1H), 1.82-1.74 (m, 1H), 1.60-1.45 (m, 1H), 1.53 (s, 9H). m/z: [ESI⁺] 534 (M+H)⁺.

Tert-butyl 6-(2-(4-(methylcarbamoyl)phenyl)benzo[d]imidazo[2,1-b]thiazole-7-carboxamido)-2-azaspiro[3.3]heptane-2-carboxylate: Starting from 2-(4-(methylcarbamoyl)phenyl)benzo[d]imidazo[2,1-b]thiazole-7-carboxylic acid (0.15 g, 0.43 mmol). Yield 0.15 g (64%), as a white solid. ¹H NMR (400 MHz, DMSO) δ 8.94 (s, 1H), 8.73 (d, J=7.4 Hz, 1H), 8.49 (d, J=1.6 Hz, 1H), 8.44 (q, J=4.6 Hz, 1H), 8.12-8.00 (m, 2H), 7.95 (d, J=8.6 Hz, 2H), 7.91 (d, J=8.6 Hz, 2H), 4.43-4.26 (m, 1H), 3.93 (s, 2H), 3.82 (s, 2H), 2.81 (d, J=4.6 Hz, 3H), 2.60-2.50 (m, 2H), 2.26 (dt, J=2.8, 8.8 Hz, 2H), 1.38 (s, 9H). m/z: [ESI⁺] 546 (M+H)⁺.

Tert-butyl 4-(2-(4-(methylcarbamoyl)phenyl)benzo[d]imidazo[2,1-b]thiazole-7-carboxamido)piperidine-1-carboxylate: Starting from 2-(4-(methylcarbamoyl)phenyl)benzo[d]imidazo[2,1-b]thiazole-7-carboxylic acid (0.10 g, 0.29 mmol). Yield 0.12 g (79%), as a white solid. ¹H NMR (400 MHz, DMSO) δ 8.92 (s, 1H), 8.50 (s, 1H), 8.48-8.40 (m, 2H), 8.07-8.03 (m, 2H), 7.95 (d, J=8.6 Hz, 2H), 7.91 (d, J=8.6 Hz, 2H), 4.07-3.98 (m, 1H), 3.95 (d, J=14.0 Hz, 2H), 3.44-3.40 (m, 2H), 2.81 (d, J=4.4 Hz, 3H), 1.83 (d, J=12.4 Hz, 2H), 1.49-1.42 (m, 2H), 1.42 (s, 9H). m/z: [ESI⁺] 534 (M+H)⁺.

Tert-butyl (S)-3-(2-(4-(methylcarbamoyl)phenyl)benzo[d]imidazo[2,1-b]thiazole-7-carboxamido)piperidine-1-carboxylate: Starting from 2-(4-(methylcarbamoyl)phenyl)benzo[d]imidazo[2,1-b]thiazole-7-carboxylic acid (0.15 g, 0.43 mmol). Yield 0.15 g (66%), as a white solid. ¹H NMR (400 MHz, DMSO) δ 8.94 (s, 1H), 8.51 (s, 1H), 8.46 (q, J=4.4 Hz, 1H), 8.41 (d, J=7.4 Hz, 1H), 8.06 (s, 2H), 7.95 (d, J=8.6 Hz, 2H), 7.92 (d, J=8.6 Hz, 2H), 4.02-3.94 (m, 1H), 3.88-3.69 (m, 2H), 3.35-3.30 (m, 2H), 2.81 (s, 3H), 1.98-1.89 (m, 1H), 1.82-1.72 (m, 1H), 1.63-1.51 (m, 1H), 1.49-1.38 (m, 1H), 1.40 (s, 9H). m/z: [ESI⁺] 534 (M+H)⁺.

Tert-butyl methyl(4-(2-(4-(methylcarbamoyl)phenyl)benzo[d]imidazo[2,1-b]thiazole-7-carboxamido)butyl)carbamate: Starting from 2-(4-(methylcarbamoyl)phenyl)benzo[d]imidazo[2,1-b]thiazole-7-carboxylic acid (0.15 g, 0.43 mmol). Yield 0.22 g (96%), as an off-white solid. ¹H NMR (400 MHz, DMSO) δ 8.94 (s, 1H), 8.61 (t, J=5.6 Hz, 1H), 8.50 (s, 1H), 8.46 (q, J=5.0 Hz, 1H), 8.08-8.02 (m, 2H), 7.95 (d, J=8.6 Hz, 2H), 7.91 (d, J=8.6 Hz, 2H), 3.19 (t, J=6.0 Hz, 2H), 2.81 (d, J=4.4 Hz, 3H), 2.77 (s, 3H), 2.55-2.50 (m, 2H), 1.56-1.48 (m, 4H), 1.38 (s, 9H). m/z: [ESI⁺] 536 (M+H)⁺.

Tert-butyl 2-((2-(4-(methylcarbamoyl)phenyl)benzo[d]imidazo[2,1-b]thiazole-7-carboxamido)methyl)-1-oxa-8-azaspiro[4.5]decane-8-carboxylate: Starting from 2-(4-(methylcarbamoyl)phenyl)benzo[d]imidazo[2,1-b]thiazole-7-carboxylic acid (0.15 g, 0.43 mmol). Yield 0.14 g (54%), as an off-white solid. ¹H NMR (400 MHz, CDCl₃) δ 8.21 (d, J=1.6 Hz, 1H), 8.09 (s, 1H), 7.93 (d, J=8.4 Hz, 2H), 7.88-7.85 (m, 1H), 7.83 (d, J=8.4 Hz, 2H), 7.69 (dd, J=1.6, 7.6 Hz, 1H), 6.61 (t, J=5.6 Hz, 1H), 6.26 (q, J=5.2 Hz, 1H), 4.22 (td, J=4.8, 9.6 Hz, 1H), 3.84 (ddd, J=3.4, 6.3, 13.6 Hz, 1H), 3.61-3.52 (m, 2H), 3.50-3.38 (m, 3H), 3.07 (d, J=5.2 Hz, 3H), 2.12 (td, J=6.4, 11.6 Hz, 1H), 1.87-1.73 (m, 3H), 1.70-1.55 (m, 4H), 1.48 (s, 9H). m/z: [ESI⁺] 604 (M+H)⁺.

Tert-butyl 6-((2-(4-(methylcarbamoyl)phenyl)benzo[d]imidazo[2,1-b]thiazole-7-carboxamido)methyl)-2-azaspiro[3.3]heptane-2-carboxylate: Starting from 2-(4-(methylcarbamoyl)phenyl)benzo[d]imidazo[2,1-b]thiazole-7-carboxylic acid (0.15 g, 0.43 mmol). Yield 0.15 g (63%), as a white solid. ¹H NMR (400 MHz, DMSO) δ 8.93 (s, 1H), 8.60 (t, J=5.8 Hz, 1H), 8.49 (d, J=1.6 Hz, 1H), 8.45 (q, J=4.4 Hz, 1H), 8.07 (d, J=8.4 Hz, 1H), 8.03 (dd, J=1.6, 8.4 Hz, 1H), 7.95 (d, J=8.6 Hz, 2H), 7.91 (d, J=8.6 Hz, 2H), 3.83 (s, 2H), 3.77 (s, 2H), 3.33-3.26 (m, 2H), 2.81 (d, J=4.4 Hz, 3H), 2.50-2.39 (m, 1H), 2.27-2.18 (m, 2H), 1.97-1.88 (m, 2H), 1.36 (s, 9H). m/z: [ESI⁺] 560 (M+H)⁺.

Tert-butyl (R)-6-((2-(4-(methylcarbamoyl)phenyl)benzo[d]imidazo[2,1-b]thiazole-7-carboxamido)methyl)-5-azaspiro[2.4]heptane-5-carboxylate: Starting from 2-(4-(methylcarbamoyl)phenyl)benzo[d]imidazo[2,1-b]thiazole-7-carboxylic acid (0.15 g, 0.43 mmol). Yield 0.12 g (50%), as a white solid. ¹H NMR (400 MHz, CD₃OD) δ 8.95 (s, 1H), 8.57 (s, 1H), 8.24 (d, J=8.4 Hz, 1H), 8.17 (d, J=8.4 Hz, 1H), 7.99 (d, J=8.6 Hz, 2H), 7.93 (d, J=8.6 Hz, 2H), 4.35-4.22 (m, 1H), 3.76-3.64 (m, 2H), 3.55 (d, J=10.4 Hz, 1H), 3.16-3.09 (m, 1H), 2.97 (s, 3H), 2.34-2.24 (m, 1H), 1.65-1.49 (m, 1H), 1.48 (s, 9H), 0.83-0.75 (m, 1H), 0.74-0.67 (m, 1H), 0.66-0.57 (m, 2H). m/z: [ESI⁺] 560 (M+H)⁺.

Tert-butyl (3-(2-(4-(methylcarbamoyl)phenyl)benzo[d]imidazo[2,1-b]thiazole-7-carboxamido)cyclohexyl)carbamate: Starting from 2-(4-(methylcarbamoyl)phenyl)benzo[d]imidazo[2,1-b]thiazole-7-carboxylic acid (0.15 g, 0.43 mmol). Yield 0.16 g (68%), as an off-white solid. ¹H NMR (400 MHz, DMSO) δ 8.94 (s, 0.7H), 8.93 (s, 0.3H), 8.51 (d, J=1.6 Hz, 0.7H), 8.50 (d, J=1.6 Hz, 0.3H), 8.44 (q, J=4.4 Hz, 0.7H), 8.41 (q, J=4.4 Hz, 0.3H), 8.26 (d, J=7.4 Hz, 1H), 8.05 (s, 2H), 7.95 (d, J=8.6 Hz, 2H), 7.91 (d, J=8.6 Hz, 2H), 6.87 (br s, 1H), 4.20 (s, 1H), 3.79 (s, 1H), 2.81 (d, J=4.4 Hz, 3H), 1.88-1.54 (m, 6H), 1.51-1.40 (m, 1H), 1.39 (s, 9H), 1.29-1.18 (m, 1H). (a mixture of cis/trans isomers with a ratio of 3:7). m/z: [ESI⁺] 548 (M+H)⁺.

Tert-butyl methyl(3-(2-(4-(methylcarbamoyl)phenyl)benzo[d]imidazo[2,1-b]thiazole-7-carboxamido)cyclobutyl)carbamate: Starting from 2-(4-(methylcarbamoyl)phenyl)benzo[d]imidazo[2,1-b]thiazole-7-carboxylic acid (0.40 g, 1.14 mmol). Yield 0.45 g (74%), as an off-white solid. ¹H NMR (400 MHz, DMSO) δ 8.94 (s, 0.8H), 8.94 (s, 0.2H), 8.89 (d, J=6.4 Hz, 1H), 8.53 (d, J=1.6 Hz, 0.8H), 8.50 (d, J=1.6 Hz, 0.2H), 8.45 (q, J=4.4 Hz, 1H), 8.11-8.04 (m, 2H), 7.95 (d, J=8.6 Hz, 2H), 7.92 (d, J=8.6 Hz, 2H), 4.93-4.69 (m, 0.8H), 4.35-4.25 (m, 1H), 4.15 (q, J=8.0 Hz, 0.2H), 2.82 (s, 3H), 2.80 (s, 3H), 2.61-2.52 (m, 2H), 2.30-2.20 (m, 2H), 1.41 (s, 9H). (a mixture of cis/trans isomers with a ratio of 1:4). m/z: [ESI⁺] 534 (M+H)+.

### Step 2 and step 3: N-((1s,3s)-3-(methylamino)cyclobutyl)-2-(m-tolyl)benzo[d]imidazo[2,1-b]thiazole-7-carboxamide formate (Compound 157) and N-((1r,3r)-3-(methylamino)cyclobutyl)-2-(m-tolyl)benzo[d]imidazo[2,1-b]thiazole-7-carboxamide formate (Compound 158)

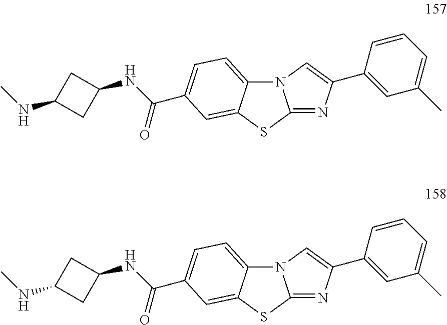

A solution of tert-butyl methyl(3-(2-(m-tolyl)benzo[d]imidazo[2,1-b]thiazole-7-carboxamido)cyclobutyl)carbamate (250 mg, 0.510 mmol) in a 4M solution of HCl (gas) in dioxane (10 mL) was stirred for 16 h at room temperature under a nitrogen atmosphere. The resulting mixture was concentrated under reduced pressure. The residue was purified by reverse phase flash chromatography with the following conditions: Column: Spherical C18, 20-40 μm, 330 g; Mobile Phase A: water (plus 10 mM formic acid); Mobile Phase B: ACN; Flow rate: 80 mL/min; Gradient: 60% B-80% B in 20 min; Detector: UV 220/254 nm. The faster eluting peak was collected, concentrated under reduced pressure and lyophilized to afford N-((1s,3s)-3-(methylamino)cyclobutyl)-2-(m-tolyl)benzo[d]imidazo[2,1-b]thiazole-7-carboxamide formate (Compound 157) as an off-white solid.

Yield 5 mg (2%). ¹H NMR (400 MHz, DMSO) δ 8.86 (d, J=7.2 Hz, 1H), 8.81 (s, 1H), 8.51 (d, J=1.2 Hz, 1H), 8.31 (s, 1H), 8.09-8.02 (m, 2H), 7.71 (d, J=1.8 Hz, 1H), 7.67 (d, J=7.6 Hz, 1H), 7.34 (dd, J=1.6, 7.6 Hz, 1H), 7.13 (d, J=7.6 Hz, 1H), 4.18-4.16 (m, 1H), 3.08-3.06 (m, 1H), 2.63-2.54 (m, 2H), 2.38 (s, 3H), 2.31 (s, 3H), 2.00 (dq, J=2.8, 8.8 Hz, 2H). m/z: [ESI⁺] 391 (M+H)⁺, (C₂₂H₂₂N₄OS).

The slower peak was collected, concentrated under reduced pressure and lyophilized to afford N-((1r,3r)-3-(methylamino)cyclobutyl)-2-(m-tolyl)benzo[d]imidazo[2,1-b]thiazole-7-carboxamide formate (Compound 158) as an off-white solid.

Yield 23 mg (10%). ¹H NMR (400 MHz, DMSO) δ 8.84 (s, 1H), 8.82 (s, 1H), 8.50 (d, J=1.2 Hz, 1H), 8.32-8.28 (m, 1H), 8.06 (d, J=1.2 Hz, 2H), 7.72 (d, J=1.8 Hz, 1H), 7.69-7.65 (m, 1H), 7.34 (dd, J=1.6, 7.6 Hz, 1H), 7.13 (d, J=7.6 Hz, 1H), 4.57-4.55 (m, 1H), 3.45-3.41 (m, 1H), 2.38 (s, 3H), 2.34 (s, 3H), 2.32-2.22 (m, 4H). m/z: [ESI⁺] 391 (M+H)⁺, (C₂₂H₂₂N₄OS).

### N-((1s,3s)-3-(piperidin-1-yl)cyclobutyl)-2-(m-tolyl)benzo[d]imidazo[2,1-b]thiazole-7-carboxamide (Compound 138)

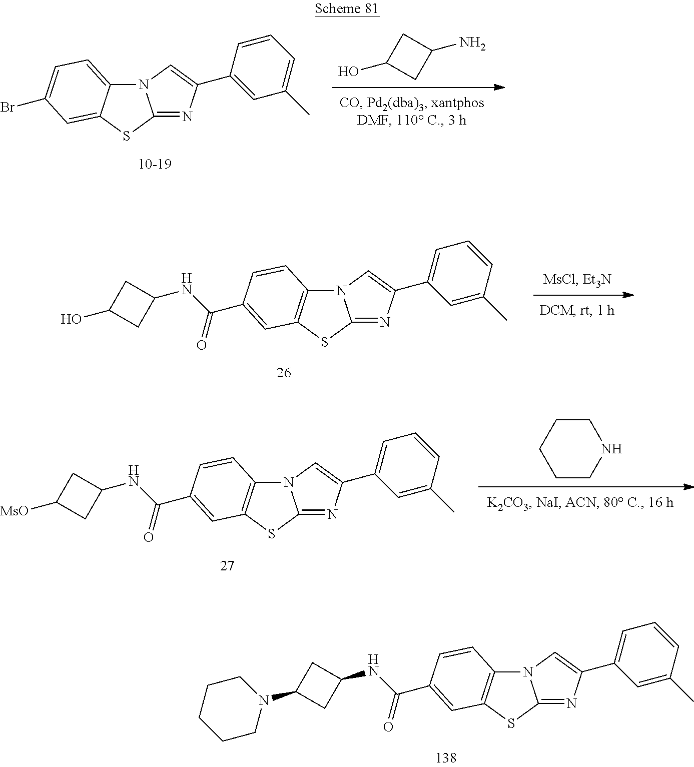 [SMILES: Cc1cccc(-c2cn3c(n2)sc2cc(Br)ccc23)c1 ; NC1CC(O)C1 ; Cc1cccc(-c2cn3c(n2)sc2cc(C(=O)NC4CC(O)C4)ccc23)c1 ; Cc1cccc(-c2cn3c(n2)sc2cc(C(=O)NC4CC(OS(C)(=O)=O)C4)ccc23)c1 ; C1CCNCC1 ; Cc1cccc(-c2cn3c(n2)sc2cc(C(=O)N[C@H]4C[C@@H](N5CCCCC5)C4)ccc23)c1]

### Step 1: N-(3-hydroxycyclobutyl)-2-(m-tolyl)benzo[d]imidazo[2,1-b]thiazole-7-carboxamide

To a stirred solution of 7-bromo-2-(m-tolyl)benzo[d]imidazo[2,1-b]thiazole (1.00 g, 2.91 mmol) in DMF (3 mL) were added 3-aminocyclobutan-1-ol (0.76 g, 8.72 mmol), 9,9-dimethyl-4,5-bis(diphenylphosphino)xanthene (XantPhos) (0.17 g, 0.29 mmol) and tris(dibenzylideneacetone)dipalladium(0) (0.53 g, 0.58 mmol) at room temperature under a nitrogen atmosphere. The resulting mixture was stirred for 3 h at 110° C. under a carbon monoxide atmosphere (balloon). After cooling down to room temperature, resulting mixture was purified by reverse phase flash chromatography with the following conditions: Column: Spherical C18, 20-40 μm, 330 g; Mobile Phase A: water (plus 10 mM NH₄HCO₃); Mobile Phase B: ACN; Flow rate: 80 mL/min; Gradient: 40% B-60% B in 20 min; Detector: UV 220/254 nm. The fractions containing desired product were collected and concentrated under reduced pressure to afford N-(3-hydroxycyclobutyl)-2-(m-tolyl)benzo[d]imidazo[2,1-b]thiazole-7-carboxamide as an off-white solid.

Yield 0.30 g (27%). ¹H NMR (400 MHz, DMSO) δ 8.80 (d, J=1.6 Hz, 1H), 8.70 (d, J=7.4 Hz, 1H), 8.49 (q, J=1.4 Hz, 1H), 8.04 (d, J=1.4 Hz, 2H), 7.70 (d, J=1.8 Hz, 1H), 7.66 (d, J=7.6 Hz, 1H), 7.32 (dd, J=1.6, 7.6 Hz, 1H), 7.15-7.08 (m, 1H), 5.14 (d, J=5.4 Hz, 0.8H), 5.07 (d, J=5.4 Hz, 0.2H), 3.98-3.83 (m, 1H), 2.61-2.53 (m, 2H), 2.37 (s, 3H), 1.94 (dq, J=2.8, 8.6 Hz, 2H). (A mixture of cis/trans isomers with a ratio of 1:4). m/z: [ESI⁺] 378 (M+H)⁺.

The compound in the table was prepared according to the procedure described above, using ethyl 6-bromo-1,3-benzothiazole-2-carboxylate as starting material on a 0.699 mmol scale.

### Step 2: 3-(2-(M-tolyl)benzo[d]imidazo[2,1-b]thiazole-7-carboxamido)cyclobutyl methanesulfonate

To a stirred solution of N-(3-hydroxycyclobutyl)-2-(m-tolyl)benzo[d]imidazo[2,1-b]thiazole-7-carboxamide (0.30 g, 0.80 mmol) in DCM (10 mL) were added triethylamine (0.16 g, 1.58 mmol) and methanesulfonyl chloride (0.11 g, 0.96 mmol). The resulting solution was stirred for 1 h at room temperature under a nitrogen atmosphere. The resulting mixture was diluted with ethyl acetate (30 mL) and washed with saturated aqueous NaHCO₃ (10 mL). The organic layer was dried over anhydrous Na₂SO₄. After filtration, the filtrate was concentrated under reduced pressure to afford 3-(2-(m-tolyl)benzo[d]imidazo[2,1-b]thiazole-7-carboxamido)cyclobutyl methanesulfonate as an off-white solid.

Yield 0.36 g (crude). ¹H NMR (400 MHz, DMSO) δ 8.88 (d, J=7.6 Hz, 1H), 8.82 (s, 1H), 8.54-8.48 (m, 1H), 8.10-8.01 (m, 2H), 7.71 (s, 1H), 7.66 (d, J=7.6 Hz, 1H), 7.33 (dd, J=1.6, 7.6 Hz, 1H), 7.12 (d, J=7.6 Hz, 1H), 4.85-4.81 (m, 1H), 4.22 (t, J=6.4 Hz, 0.2H), 4.17 (q, J=8.0 Hz, 0.8 Hz), 3.19 (s, 0.6H), 3.18 (s, 2.4H), 2.89-2.77 (m, 2H), 2.43-2.35 (m, 2H), 2.37 (s, 3H). (A mixture of cis/trans isomers with a ratio of 1:4). m/z: [ESI⁺] 456 (M+H)⁺.

### Step 3: N-((1s,3s)-3-(piperidin-1-yl)cyclobutyl)-2-(m-tolyl)benzo[d]imidazo[2,1-b]thiazole-7-carboxamide (Compound 138)

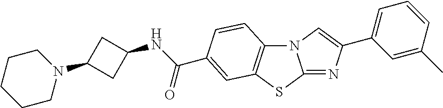 [SMILES: Cc1cccc(-c2cn3c(n2)sc2cc(C(=O)N[C@H]4C[C@@H](N5CCCCC5)C4)ccc23)c1]

To a stirred solution of 3-(2-(m-tolyl)benzo[d]imidazo[2,1-b]thiazole-7-carboxamido)cyclobutyl methanesulfonate (360 mg, 0.790 mmol) in acetonitrile (12 mL) were added piperidine (74 mg, 0.869 mmol), K₂CO₃ (220 mg, 1.592 mmol) and NaI (12 mg, 0.080 mmol) at room temperature under a nitrogen atmosphere. The resulting mixture was stirred for overnight at 80° C. under a nitrogen atmosphere. After cooling down to room temperature, the resulting mixture was filtered and the filtrates were concentrated under reduced pressure. The residue was purified by reverse phase flash chromatography with the following conditions: Column: Spherical C18, 20-40 μm, 330 g; Mobile Phase A: water (plus 10 mM NH₄HCO₃); Mobile Phase B: ACN; Flow rate: 80 mL/min; Gradient: 45% B-65% B in 20 min; Detector: UV 220/254 nm. The fractions containing desired product were collected and concentrated under reduced pressure to afford N-((1s,3s)-3-(piperidin-1-yl)cyclobutyl)-2-(m-tolyl)benzo[d]imidazo[2,1-b]thiazole-7-carboxamide as an off-white solid.

Yield 24 mg (7%). ¹H NMR (400 MHz, DMSO) δ 8.81 (s, 1H), 8.73 (d, J=7.8 Hz, 1H), 8.50 (s, 1H), 8.05 (d, J=2.2 Hz, 2H), 7.71 (s, 1H), 7.67 (d, J=7.8 Hz, 1H), 7.33 (dd, J=1.6, 7.6 Hz, 1H), 7.13 (d, J=7.6 Hz, 1H), 4.26-4.13 (m, 1H), 2.47-2.41 (m, 3H), 2.38 (s, 3H), 2.35-2.21 (m, 4H), 1.98-1.86 (m, 2H), 1.57-1.47 (m, 4H), 1.44-1.34 (m, 2H). m/z: [ESI⁺] 445 (M+H)⁺. (C₂₆H₂₈N₄OS)

### N-(3-(diethylamino)propyl)-2-(4-(2-oxopyrrolidin-1-yl)phenyl)benzo[d]imidazo[2,1-b]thiazole-7-carboxamide (Compound 179)

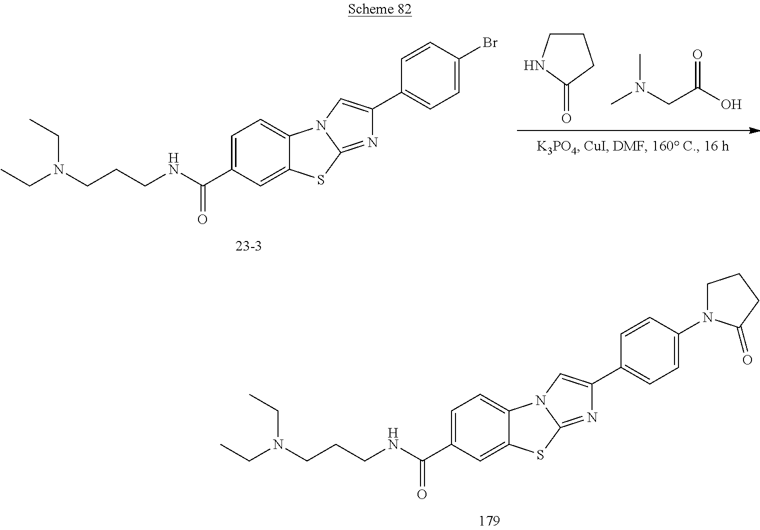

To a stirred solution of 2-(4-bromophenyl)-N-(3-(diethylamino)propyl)benzo[d]imidazo[2,1-b]thiazole-7-carboxamide (150 mg, 0.309 mmol) in DMF (6 mL) were added pyrrolidone (53 mg, 0.623 mmol), K₃PO₄ (197 mg, 0.928 mmol), 2-(dimethylamino)acetic acid (3 mg, 0.029 mmol) and CuI (6 mg, 0.032 mmol) at room temperature under a nitrogen atmosphere. The resulting mixture was stirred for 16 h at 160° C. under a nitrogen atmosphere. After cooling down to room temperature, the resulting mixture was diluted with water (4 mL). The precipitated solids were collected by filtration and washed with water (3×3 mL). The crude product was purified by Prep-CHIRAL-SFC with the following conditions (Column: Triart Diol-NP, 20×250 mm, 5 μm; Mobile Phase A: CO₂; Mobile Phase B: methanol (plus 0.5% 2 M NH₃ in methanol); Flow rate: 50 mL/min; Gradient: 35% B for 10 min; Detector: UV 220/254 nm. The fractions containing desired product were collected and concentrated under reduced pressure to afford N-(3-(diethylamino)propyl)-2-(4-(2-oxopyrrolidin-1-yl)phenyl)benzo[d]imidazo[2,1-b]thiazole-7-carboxamide as an off-white solid.

Yield: 58 mg (38%). ¹H NMR (400 MHz, DMSO) δ 8.78 (s, 1H), 8.62 (t, J=5.4 Hz, 1H), 8.48 (d, J=1.6 Hz, 1H), 8.05 (d, J=8.4 Hz, 1H), 8.01 (dd, J=1.6, 8.4 Hz, 1H), 7.87 (d, J=8.8 Hz, 2H), 7.75 (d, J=8.8 Hz, 2H), 3.88 (t, J=7.0 Hz, 2H), 3.37-3.29 (m, 2H), 2.59-2.41 (m, 8H), 2.10-2.08 (m, 2H), 1.68-1.66 (m, 2H), 0.96 (t, J=7.0 Hz, 6H). m/z: [ESI⁺] 490 (M+H)⁺, (C₂₇H₃₁N₅O₂S).

### 2-(4-cyanophenyl)-N-(3-(diethylamino)propyl)benzo[d]imidazo[2,1-b]thiazole-7-carboxamide Compound 160)

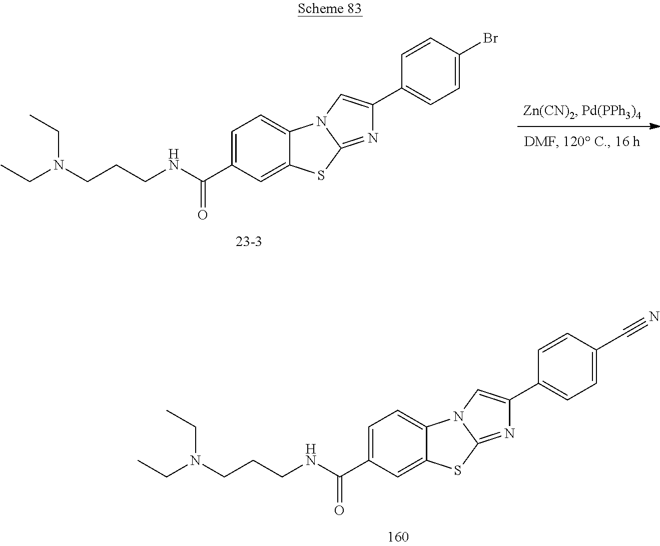 [SMILES: CCN(CC)CCCNC(=O)c1ccc2c(c1)sc1nc(-c3ccc(Br)cc3)cn12 ; CCN(CC)CCCNC(=O)c1ccc2c(c1)sc1nc(-c3ccc(C#N)cc3)cn12]

To a stirred solution of 2-(4-bromophenyl)-N-(3-(diethylamino)propyl)benzo[d]imidazo[2,1-b]thiazole-7-carboxamide (0.30 g, 0.62 mmol) in DMF (10 mL) were added zinc cyanide (0.11 g, 0.94 mmol) and tetrakis(triphenylphosphine)palladium(0) (0.21 g, 0.18 mmol) at room temperature under a nitrogen atmosphere. The resulting mixture stirred for 16 h at 120° C. under a nitrogen atmosphere. Upon completion, the resulting mixture was cooled down to room temperature and concentrated under reduced pressure. The residue was purified by Prep-HPLC with the following conditions (Column: XBridge Prep OBD C18 Column, 30×150 mm, 5 um; Mobile Phase A: water (plus 10 mM NH₄HCO₃); Mobile Phase B: ACN; Flow rate: 60 mL/min; Gradient: 32% B to 45% B in 7 min; Detector: UV 220/254 nm. The fractions containing desired product were collected, concentrated under reduced pressure and lyophilized to afford 2-(4-cyanophenyl)-N-(3-(diethylamino)propyl)benzo[d]imidazo[2,1-b]thiazole-7-carboxamide as an off-white solid.

Yield: 20 mg (7%). ¹H NMR (400 MHz, DMSO) δ 9.04 (s, 1H), 8.64 (t, J=5.4 Hz, 1H), 8.50 (d, J=1.6 Hz, 1H), 8.08-8.01 (m, 4H), 7.91 (d, J=8.4 Hz, 2H), 3.34-3.26 (m, 2H), 2.50-2.43 (m, 6H), 1.68-1.66 (m, 2H), 0.96 (t, J=7.0 Hz, 6H). m/z: [ESI⁺] 432 (M+H)⁺, (C₂₄H₂₅N₅OS).

### N-(3-(diethylamino)propyl)-2-morpholinobenzo[d]imidazo[2,1-b]thiazole-7-carboxamide formate (Compound 162)

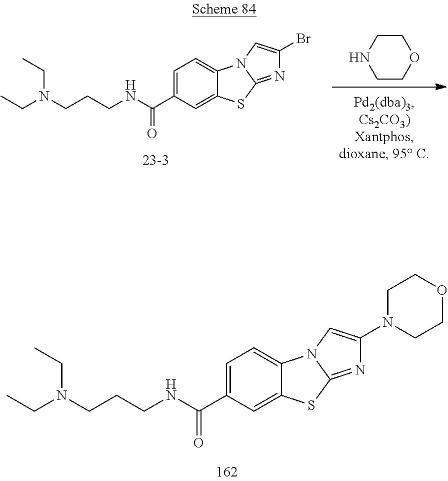 [SMILES: CCN(CC)CCCNC(=O)c1ccc2c(c1)sc1nc(Br)cn12 ; C1COCCN1 ; CCN(CC)CCCNC(=O)c1ccc2c(c1)sc1nc(N3CCOCC3)cn12]

To a stirred solution of 2-bromo-N-(3-(diethylamino)propyl)benzo[d]imidazo[2,1-b]thiazole-7-carboxamide (0.50 g, 1.22 mmol) in dioxane (15 mL) were added Cs₂CO₃ (1.59 g, 4.88 mmol), morpholine (0.21 g, 2.41 mmol), tris(dibenzylideneacetone)dipalladium(II) (0.11 g, 0.12 mmol) and 9,9-dimethyl-4,5-bis(diphenylphosphino)xanthene (Xantphos) (0.14 g, 0.24 mmol). The resulting mixture was stirred for 16 at 95° C. After cooling down to room temperature, the resulting mixture was diluted with water (100 mL) and extracted with ethyl acetate (3×100 mL). The combined organic layers were concentrated under reduced pressure. The residue was purified by reverse phase flash chromatography with the following conditions: Column: Spherical C18, 20-40 μm, 330 g; Mobile Phase A: water (plus 10 mM formic acid); Mobile Phase B: ACN; Flow rate: 80 mL/min; Gradient: 10% B-30% B in 20 min; Detector: UV 220/254 nm. The fractions containing desired product were collected and concentrated under reduced pressure to afford N-(3-(diethylamino)propyl)-2-morpholinobenzo[d]imidazo[2,1-b]thiazole-7-carboxamide as a brown solid.

Yield: 17 mg (3%). ¹H NMR (400 MHz, DMSO) δ 8.64 (t, J=5.6 Hz, 1H), 8.43 (s, 1H), 8.25 (s, 1H), 7.97 (dd, J=1.6, 8.4 Hz, 1H), 7.91 (d, J=8.4 Hz, 1H), 7.54 (s, 1H), 3.75 (t, J=4.8 Hz, 4H), 3.38-3.28 (m, 2H), 3.10 (t, J=4.8 Hz, 4H), 2.69-2.55 (m, 6H), 1.74-1.72 (m, 2H), 1.02 (t, J=7.2 Hz, 6H). m/z: [ESI⁺] 416 (M+H)⁺. (C₂₁H₂₉N₅O₂S).

### N-(3-(diethylamino)propyl)-2-(4-methylpyridin-2-yl)benzo[d]imidazo[2,1-b]thiazole-7-carboxamide hemiformate (Compound 194)

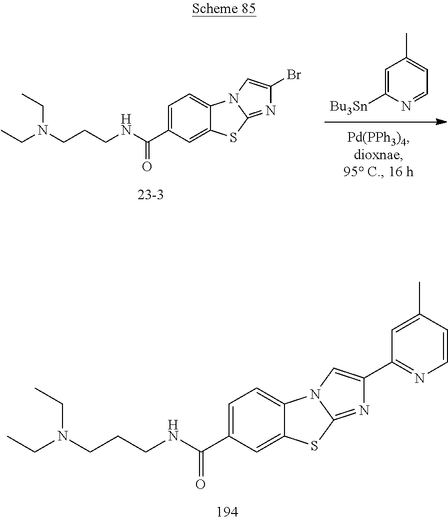

To a stirred solution of 2-bromo-N-(3-(diethylamino)propyl)benzo[d]imidazo[2,1-b]thiazole-7-carboxamide (300 mg, 0.733 mmol) in dioxane (4 mL) were added 4-methyl-2-(tributylstannyl)pyridine (280 mg, 0.733 mmol) and tetrakis(triphenylphosphine)palladium(0) (140 mg, 0.125 mmol) at room temperature under a nitrogen atmosphere. The resulting mixture was stirred for 16 h at 95° C. After cooling down to room temperature, the resulting mixture was diluted with water (100 mL) and extracted with ethyl acetate (3×100 mL). The combined organic layer was concentrated under reduced pressure. The residue was purified by reverse phase flash chromatography with the following conditions: Column: Spherical C18, 20-40 μm, 330 g; Mobile Phase A: water (plus 10 mM formic acid); Mobile Phase B: ACN; Flow rate: 80 mL/min; Gradient: 10% B-30% B in 20 min; Detector: UV 220/254 nm. The fractions containing desired product were collected, concentrated under reduced pressure and lyophilized to afford N-(3-(diethylamino)propyl)-2-(4-methylpyridin-2-yl)benzo[d]imidazo[2,1-b]thiazole-7-carboxamide as an off-white solid.

Yield: 15 mg (5%). ¹H NMR (400 MHz, DMSO) δ 8.91 (s, 1H), 8.66 (t, J=5.6 Hz, 1H), 8.49 (d, J=1.6 Hz, 1H), 8.47-8.41 (m, 1H), 8.22 (d, J=8.4 Hz, 1H), 8.00 (dd, J=1.6, 8.4 Hz, 1H), 7.89-7.78 (m, 1H), 7.14 (dd, J=1.6, 5.2 Hz, 1H), 3.33 (q, J=6.6 Hz, 2H), 2.60-2.50 (m, 6H), 2.39 (s, 3H), 1.72-1.69 (m, 2H), 0.99 (t, J=7.2 Hz, 6H). m/z: [ESI⁺] 422 (M+H)⁺, (C₂₃H₂₇N₅OS). 2-(4-(1H-imidazol-2-yl)phenyl)-N-(3-(diethylamino)propyl)benzo[d]imidazo[2,1-b]thiazole-7-carboxamide (Compound 268)

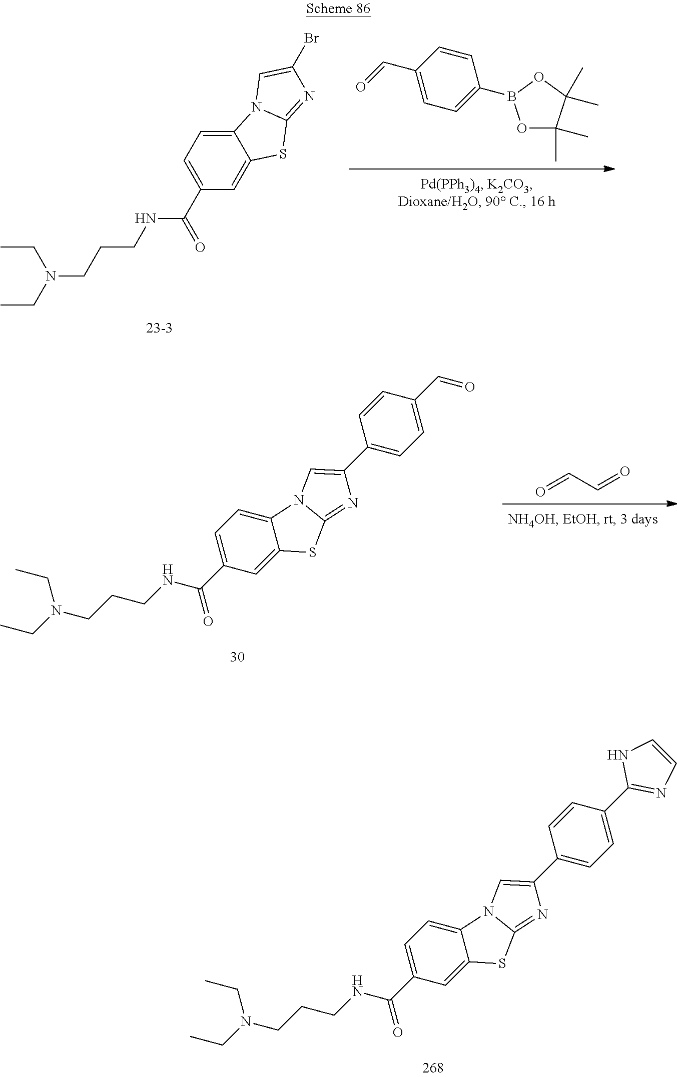

### Step 1: N-(3-(diethylamino)propyl)-2-(4-formylphenyl)benzo[d]imidazo[2,1-b]thiazole-7-carboxamide

To a stirred solution of 2-(4-bromophenyl)-N-(3-(diethylamino)propyl)benzo[d]imidazo[2,1-b]thiazole-7-carboxamide (3.00 g, 7.33 mmol) in dioxane (16 mL) were added water (4 mL), 4-(4,4,5,5-tetramethyl-1,3,2-dioxaborolan-2-yl)benzaldehyde (2.55 g, 10.99 mmol), K₂CO₃ (3.04 g, 22.00 mmol) and tetrakis(triphenylphosphine)palladium(0) (0.85 g, 0.74 mmol) at room temperature under a nitrogen atmosphere. After stirring for 16 h at 90° C. under a nitrogen atmosphere, the resulting mixture was cooled down to room temperature and filtered. The filtrate was concentrated under reduced pressure. The residue was purified by reverse phase flash chromatography with the following conditions: Column: Spherical C18, 20-40 μm, 330 g; Mobile Phase A: water (plus 10 mM NH₄HCO₃); Mobile Phase B: ACN; Flow rate: 80 mL/min; Gradient: 60% B-80% B in 20 min; Detector: UV 220/254 nm. The fractions containing desired product were collected and concentrated under reduced pressure to afford N-(3-(diethylamino)propyl)-2-(4-formylphenyl)benzo[d]imidazo[2,1-b]thiazole-7-carboxamide as an off-white solid.

Yield: 2.00 (63%). ¹H NMR (400 MHz, DMSO) δ 10.00 (s, 1H), 9.02 (s, 1H), 8.63 (t, J=5.4 Hz, 1H), 8.49 (d, J=1.6 Hz, 1H), 8.08 (d, J=8.0 Hz, 2H), 8.06 (d, J=8.4 Hz, 1H), 8.02 (dd, J=1.6, 8.4 Hz, 1H), 7.98 (d, J=8.0 Hz, 2H), 3.30-3.25 (m, 2H), 2.50-2.42 (m, 6H), 1.68-1.66 (m, 2H), 0.95 (t, J=7.0 Hz, 6H). m/z: [ESI⁺] 435 (M+H)⁺.

The following compound below were synthesized according to the described procedure above, using 2-bromo-N-(3-(diethylamino)propyl)benzo[d]imidazo[2,1-b]thiazole-7-carboxamide as the starting material on a 2.44 mmol scale.

4-(7-((3-(Diethylamino)propyl)carbamoyl)benzo[d]imidazo[2,1-b]thiazol-2-yl)-3-fluorobenzoic acid: Yield 0.60 g (52%), as an off-white solid. ¹H NMR (400 MHz, DMSO) δ 8.76 (t, J=5.6 Hz, 1H), 8.69 (d, J=3.6 Hz, 1H), 8.47 (d, J=1.6 Hz, 1H), 8.20 (d, J=8.4 Hz, 1H), 8.12 (dd, J=1.6, 8.0 Hz, 1H), 7.99 (dd, J=1.6, 8.4 Hz, 1H), 7.77 (d, J=8.2 Hz, 1H), 7.66 (d, J=12.0 Hz, 1H), 3.36 (q, J=6.4 Hz, 2H), 2.86-2.75 (m, 6H), 1.85-1.83 (m, 2H), 1.09 (t, J=7.2 Hz, 6H). m/z: [ESI⁺] 469 (M+H)⁺.

Tert-butyl (4-(7-((3-(diethylamino)propyl)carbamoyl)benzo[d]imidazo[2,1-b]thiazol-2-yl)benzyl)carbamate formate: Yield 145 mg (34%), as an off-white solid. ¹H NMR (400 MHz, DMSO) δ 8.77 (s, 1H), 8.75 (t, J=5.6 Hz, 1H), 8.50 (s, 1H), 8.31 (s, 1H), 8.04 (s, 2H), 7.82 (d, J=8.0 Hz, 2H), 7.42 (dd, J=1.6, 6.2 Hz, 1H), 7.31 (d, J=8.0 Hz, 2H), 4.16 (d, J=6.2 Hz, 2H), 3.36 (q, J=6.4 Hz, 2H), 2.86-2.73 (m, 6H), 1.82-1.80 (m, 2H), 1.41 (s, 9H), 1.08 (t, J=7.2 Hz, 6H). m/z: [ESI+] 536 (M+H)+.

### Step 2: 2-(4-(1H-imidazol-2-yl)phenyl)-N-(3-(diethylamino)propyl)benzo[d]imidazo[2,1-b]thiazole-7-carboxamide hemiformate (Compound 268)

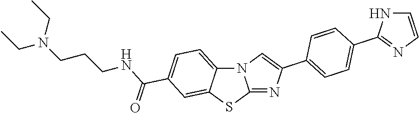 [SMILES: CCN(CC)CCCNC(=O)c1ccc2c(c1)sc1nc(-c3ccc(-c4ncc[nH]4)cc3)cn12]

To a stirred solution of N-(3-(diethylamino)propyl)-2-(4-formylphenyl)benzo[d]imidazo[2,1-b]thiazole-7-carboxamide (300 mg, 0.690 mmol) in ethanol (5 mL) were added oxalaldehyde (45 mg, 0.775 mmol) and a 25% solution of NH₄OH in water (2.40 g, 16.90 mmol) at room temperature under a nitrogen atmosphere. The resulting mixture was stirred for 3 days at room temperature under a nitrogen atmosphere. The resulting mixture was concentrated under reduced pressure. The residue was purified by Prep-HPLC with the following conditions: Column: Sunfire prep C18 column, 30×150 mm, 5 μm; Mobile Phase A: water (plus 0.1% formic acid, v/v); zw Mobile Phase B: ACN; Flow rate: 60 mL/min; Gradient: 3% B to 20% B in 8 min; Detector: UV 220/254 nm. The fractions containing desired product were collected and concentrated under reduced pressure to afford 2-(4-(1H-imidazol-2-yl)phenyl)-N-(3-(diethylamino)propyl)benzo[d]imidazo[2,1-b]thiazole-7-carboxamide hemiformate as a light brown solid.

Yield: 20 mg (6%). ¹H NMR (400 MHz, DMSO) δ 12.44 (br s, 1H), 08.87 (s, 1H), 8.67 (t, J=5.4 Hz, 1H), 8.50 (d, J=1.6 Hz, 1H), 8.06 (d, J=8.4 Hz, 1H), 8.04 (dd, J=1.6, 8.4 Hz, 1H), 8.02 (d, J=8.2 Hz, 2H), 7.94 (d, J=8.2 Hz, 2H), 7.16 (s, 2H), 3.34 (q, J=6.4 Hz, 2H), 2.65-2.58 (m, 6H), 1.74-1.72 (m, 2H), 1.00 (t, J=7.0 Hz, 6H). m/z: [ESI⁺] 473 (M+H)⁺, (C₂₆H₂₈N₆OS).

### 1-(2-(diethylamino)ethyl)-3-(2-(m-tolyl)benzo[d]imidazo[2,1-b]thiazol-7-yl)urea formate (Compound 267)

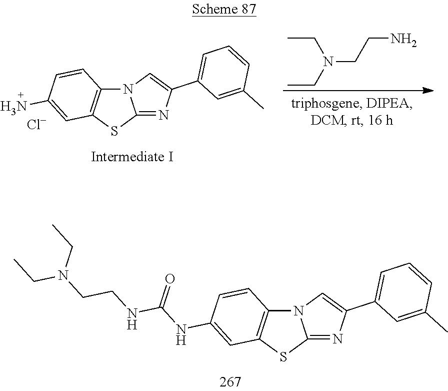

To a stirred solution of 2-(m-tolyl)benzo[d]imidazo[2,1-b]thiazol-7-aminium chloride (150 mg, 0.475 mmol) in DCM (10 mL) were added DIPEA (123 mg, 0.952 mmol), triphosgene (70 mg, 0.236 mmol). The resulting mixture was stirred for 30 min at 0° C. under a nitrogen atmosphere, followed by the addition of (2-aminoethyl)diethylamine (110 mg, 0.947 mmol). The resulting mixture was stirred for additional 16 h at room temperature. The resulting mixture was quenched with saturated aqueous NaHCO₃ (20 mL) and extracted with ethyl acetate (3×20 mL). The combined organic layers were concentrated under reduced pressure. The residue was purified by reverse phase flash chromatography with the following conditions: Column: Spherical C18, 20-40 μm, 120 g; Mobile Phase A: water (plus 0.1% formic acid, v/v); Mobile Phase B: ACN; Flow rate: 60 mL/min; Gradient: 30% B-50% B in 20 min; Detector: UV 220/254 nm. The fractions containing desired product were collected and concentrated under reduced pressure to afford 1-(2-(diethylamino)ethyl)-3-(2-(m-tolyl)benzo[d]imidazo[2,1-b]thiazol-7-yl)urea formate as a light yellow solid.

Yield: 95 mg (44%). ¹H NMR (400 MHz, DMSO-d₆) δ 9.13 (br s, 1H), 8.65 (s, 1H), 8.12 (d, J=2.0 Hz, 1H), 7.82 (d, J=8.6 Hz, 1H), 7.69 (d, J=2.0 Hz, 1H), 7.65 (d, J=7.6 Hz, 1H), 7.48 (dd, J=2.0, 8.8 Hz, 1H), 7.31 (t, J=7.6 Hz, 1H), 7.09 (d, J=7.6 Hz, 1H), 6.39 (br s, 1H), 3.23 (t, J=6.0 Hz, 2H), 2.68-2.57 (m, 6H), 2.37 (s, 3H), 1.08-0.91 (m, 6H). m/z: [ESI⁺] 422 (M+H)⁺, (C₂₃H₂₇N₅OS).

### General Suzuki Coupling Procedure

To a stirred solution of 2-(4-bromophenyl)-N-(3-(diethylamino)propyl)benzo[d]imidazo[2,1-b]thiazole-7-carboxamide (1.00 eq.) in dioxane (0.46 M) were added water (4:1, v/v), borate or boronic acid (1.50 eq.), K₂CO₃ (0.67 eq.) and tetrakis(triphenylphosphine)palladium(0) (0.10 eq.) at room temperature under a nitrogen atmosphere.

After stirring for 16 h at 90° C. under a nitrogen atmosphere, the resulting mixture was cooled down to room temperature and filtered. The filtrate was concentrated under reduced pressure. The residue was purified by reverse phase flash chromatography with the addition of NH₄HCO₃ to produce the parent product while with the addition of formic acid, to produce the product as formate form. The fractions containing desired product were collected and concentrated under reduced pressure to afford the corresponding compounds.

### Analytical Data for Compounds Prepared According to the Methods Described Above

### N-(3-(diethylamino)propyl)-2-(4-(2,2,2-trifluoro-1-(methylamino)ethyl)phenyl)benzo[d]imidazo[2,1-b]thiazole-7-carboxamide (Compound 218)

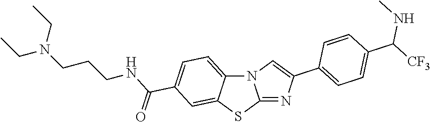 [SMILES: CCN(CC)CCCNC(=O)c1ccc2c(c1)sc1nc(-c3ccc(C(NC)C(F)(F)F)cc3)cn12]

Starting from 2-bromo-N-(3-(diethylamino)propyl)benzo[d]imidazo[2,1-b]thiazole-7-carboxamide (300 mg, 0.733 mmol). Yield 18 mg (5%), as an off-white solid.

¹H NMR (400 MHz, DMSO) δ 8.85 (s, 1H), 8.63 (t, J=5.6 Hz, 1H), 8.49 (d, J=1.6 Hz, 1H), 8.07 (d, J=8.4 Hz, 1H), 8.02 (dd, J=1.6, 8.4 Hz, 1H), 7.90 (d, J=8.0 Hz, 2H), 7.54 (d, J=8.0 Hz, 2H), 4.29 (s, 1H), 3.37-3.28 (m, 2H), 2.85 (s, 1H), 2.50-2.46 (m, 6H), 2.26 (s, 3H), 1.69-1.67 (m, 2H), 0.96 (t, J=7.2 Hz, 6H). m/z: [ESI⁺] 518 (M+H)⁺, (C₂₆H₃₀F₃N₅OS).

### N-(3-(diethylamino)propyl)-2-(pyridin-4-yl)benzo[d]imidazo[2,1-b]thiazole-7-carboxamide (Compound 161)

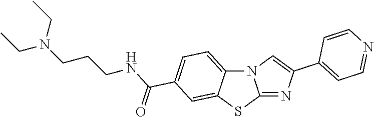 [SMILES: CCN(CC)CCCNC(=O)c1ccc2c(c1)sc1nc(-c3ccncc3)cn12]

Starting from 2-bromo-N-(3-(diethylamino)propyl)benzo[d]imidazo[2,1-b]thiazole-7-carboxamide (200 mg, 0.489 mmol). Yield 35 mg (18%), as an off-white solid.

¹H NMR (400 MHz, DMSO) δ 9.09 (s, 1H), 8.65 (t, J=5.6 Hz, 1H), 8.62 (d, J=6.2 Hz, 2H), 8.51 (d, J=1.6 Hz, 1H), 8.08 (d, J=8.4 Hz, 1H), 8.03 (dd, J=1.6, 8.4 Hz, 1H), 7.81 (d, J=6.2 Hz, 2H), 3.31 (d, J=6.3 Hz, 2H), 2.55-2.50 (m, 6H), 1.69 (p, J=7.0 Hz, 2H), 0.97 (t, J=7.2 Hz, 6H). m/z: [ESI⁺] 408 (M+H)⁺, (C₂₂H₂₅N₅OS).

### 4-(7-((3-(diethylamino)propyl)carbamoyl)benzo[d]imidazo[2,1-b]thiazol-2-yl)benzoic acid hemiformate (Compound 172)

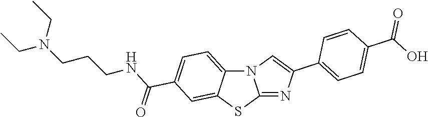 [SMILES: CCN(CC)CCCNC(=O)c1ccc2c(c1)sc1nc(-c3ccc(C(=O)O)cc3)cn12]

Starting from 2-(4-bromophenyl)-N-(3-(diethylamino)propyl)benzo[d]imidazo[2,1-b]thiazole-7-carboxamide (200 mg, 0.489 mmol). Yield 18 mg (8%), as an off-white solid.

¹H NMR (400 MHz, DMSO) δ 8.97 (s, 1H), 8.66 (t, J=5.6 Hz, 1H), 8.50 (d, J=1.6 Hz, 1H), 8.07 (d, J=8.4 Hz, 1H), 8.03 (dd, J=1.6, 8.4 Hz, 1H), 8.01 (d, J=8.6 Hz, 2H), 7.99 (d, J=8.6 Hz, 2H), 3.40-3.30 (m, 2H), 2.60-2.50 (m, 6H), 1.72-1.70 (m, 2H), 0.99 (t, J=7.2 Hz, 6H). m/z: [ESI⁺]451 (M+H)⁺, (C₂₄H₂₆N₄O₃S).

### N-(3-(diethylamino)propyl)-2-(3-isopropylphenyl)benzo[d]imidazo[2,1-b]thiazole-7-carboxamide hemiformate (Compound 169)

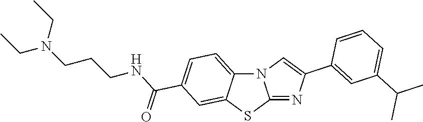 [SMILES: CCN(CC)CCCNC(=O)c1ccc2c(c1)sc1nc(-c3cccc(C(C)C)c3)cn12]

Starting from 2-bromo-N-(3-(diethylamino)propyl)benzo[d]imidazo[2,1-b]thiazole-7-carboxamide (300 mg, 0.733 mmol). Yield 142 mg (41%), as an off-white solid.

¹H NMR (400 MHz, DMSO) δ 8.83 (s, 1H), 8.65 (t, J=5.6 Hz, 1H), 8.49 (d, J=1.6 Hz, 1H), 8.06 (d, J=8.4 Hz, 1H), 8.02 (dd, J=1.6, 8.4 Hz, 1H), 7.78 (d, J=1.8 Hz, 1H), 7.68 (dd, J=1.4, 7.8 Hz, 1H), 7.36 (d, J=7.6 Hz, 1H), 7.20 (d, J=7.6 Hz, 1H), 3.33 (q, J=6.4 Hz, 2H), 3.01-2.88 (m, 1H), 2.59-2.51 (m, 6H), 1.71-1.69 (m, 2H), 1.27 (d, J=7.0 Hz, 6H), 0.98 (t, J=7.2 Hz, 6H). m/z: [ESI⁺] 449 (M+H)⁺, (C₂₆H₃₂N₄OS).

### N-(3-(diethylamino)propyl)-2-(3-morpholinophenyl)benzo[d]imidazo[2,1-b]thiazole-7-carboxamide hemiformate (Compound 170)

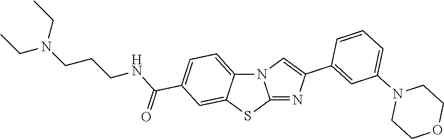 [SMILES: CCN(CC)CCCNC(=O)c1ccc2c(c1)sc1nc(-c3cccc(N4CCOCC4)c3)cn12]

Starting from 2-bromo-N-(3-(diethylamino)propyl)benzo[d]imidazo[2,1-b]thiazole-7-carboxamide (300 mg, 0.733 mmol). Yield 28 mg (8%), white solid.

¹H NMR (400 MHz, DMSO) δ 8.82 (s, 1H), 8.64 (t, J=5.6 Hz, 1H), 8.48 (s, 1H), 8.03 (s, 2H), 7.47 (d, J=2.0 Hz, 1H), 7.35-7.28 (m, 2H), 6.91 (dd, J=2.0, 7.8 Hz, 1H), 3.78 (dd, J=3.6, 6.0 Hz, 4H), 3.33-3.26 (m, 2H), 3.18 (dd, J=3.6, 6.0 Hz, 4H), 2.60-2.50 (m, 6H), 1.72-1.70 (m, 2H), 0.99 (t, J=7.2 Hz, 6H). m/z: [ESI⁺] 492 (M+H)⁺, (C₂₇H₃₃N₅O₂S).

### N-(3-(diethylamino)propyl)-2-(5-methylpyridin-3-yl)benzo[d]imidazo[2,1-b]thiazole-7-carboxamide hemiformate (Compound 165)

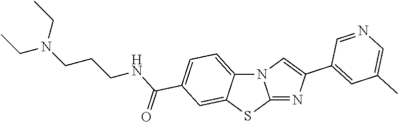 [SMILES: CCN(CC)CCCNC(=O)c1ccc2c(c1)sc1nc(-c3cncc(C)c3)cn12]

Starting from 2-bromo-N-(3-(diethylamino)propyl)benzo[d]imidazo[2,1-b]thiazole-7-carboxamide (300 mg, 0.733 mmol). Yield 19 mg (6%), white solid.

¹H NMR (400 MHz, DMSO) δ 8.93 (s, 1H), 8.89 (d, J=2.0 Hz, 1H), 8.66 (t, J=5.6 Hz, 1H), 8.50 (d, J=1.2 Hz, 1H), 8.36 (d, J=2.0 Hz, 1H), 8.07-8.02 (m, 3H), 3.33-3.26 (m, 2H), 2.58-2.50 (m, 6H), 2.37 (s, 3H), 1.71-1.69 (m, 2H), 0.98 (t, J=7.2 Hz, 6H). m/z: [ESI⁺] 422 (M+H)⁺, (C₂₃H₂₇N₅OS).

### N-(3-(diethylamino)propyl)-2-(3-(pyrrolidin-1-yl)phenyl)benzo[d]imidazo[2,1-b]thiazole-7-carboxamide formate (Compound 171)

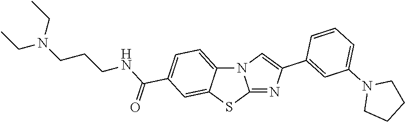 [SMILES: CCN(CC)CCCNC(=O)c1ccc2c(c1)sc1nc(-c3cccc(N4CCCC4)c3)cn12]

Starting from 2-bromo-N-(3-(diethylamino)propyl)benzo[d]imidazo[2,1-b]thiazole-7-carboxamide (300 mg, 0.733 mmol). Yield 79 mg (21%), white solid.

¹H NMR (400 MHz, DMSO) δ 8.77 (s, 1H), 8.65 (t, J=5.6 Hz, 1H), 8.48 (d, J=1.6 Hz, 1H), 8.06 (d, J=8.4 Hz, 1H), 8.02 (dd, J=1.6, 8.4 Hz, 1H), 7.22 (dd, J=1.6, 7.8 Hz, 1H), 7.13-7.09 (m, 1H), 7.09 (dd, J=1.6, 2.0 Hz, 1H), 6.50 (dd, J=2.4, 8.0 Hz, 1H), 3.40-3.31 (m, 6H), 2.62-2.52 (m, 6H), 2.05-1.93 (m, 4H), 1.73-1.71 (m, 2H), 1.00 (t, J=7.2 Hz, 6H). m/z: [ESI⁺] 476 (M+H)⁺, (C₂₇H₃₃N₅OS).

### N-(3-(diethylamino)propyl)-2-(3-(methylcarbamoyl)phenyl)benzo[d]imidazo[2,1-b]thiazole-7-carboxamide (Compound 167)

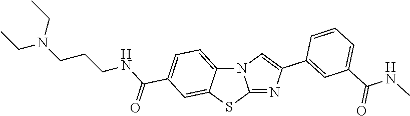 [SMILES: CCN(CC)CCCNC(=O)c1ccc2c(c1)sc1nc(-c3cccc(C(=O)NC)c3)cn12]

Starting from 2-bromo-N-(3-(diethylamino)propyl)benzo[d]imidazo[2,1-b]thiazole-7-carboxamide (150 mg, 0.366 mmol). Yield 23 mg (14%), white solid.

¹H NMR (400 MHz, DMSO) δ 8.90 (s, 1H), 8.64 (t, J=5.6 Hz, 1H), 8.54 (q, J=4.4 Hz, 1H), 8.50 (d, J=1.6 Hz, 1H), 8.37 (d, J=1.8 Hz, 1H), 8.09 (d, J=8.4 Hz, 1H), 8.03 (dd, J=1.6, 8.4 Hz, 1H), 8.01-7.98 (m, 1H), 7.78-7.73 (m, 1H), 7.54 (d, J=7.6 Hz, 1H), 3.33-3.26 (m, 2H), 2.83 (d, J=4.4 Hz, 3H), 2.58-2.51 (m, 6H), 1.70-1.68 (m, 2H), 0.97 (t, J=7.2 Hz, 6H). m/z: [ESI⁺] 464 (M+H)⁺, (C₂₅H₂₉N₅O₂S).

### N-(3-(diethylamino)propyl)-2-(4-(oxetan-3-yl)phenyl)benzo[d]imidazo[2,1-b]thiazole-7-carboxamide (Compound 173)

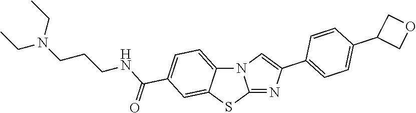 [SMILES: CCN(CC)CCCNC(=O)c1ccc2c(c1)sc1nc(-c3ccc(C4COC4)cc3)cn12]

Starting from 2-bromo-N-(3-(diethylamino)propyl)benzo[d]imidazo[2,1-b]thiazole-7-carboxamide (300 mg, 0.733 mmol). Yield 55 mg (16%), white solid.

¹H NMR (400 MHz, DMSO) δ 8.82 (s, 1H), 8.63 (t, J=5.6 Hz, 1H), 8.48 (d, J=1.6 Hz, 1H), 8.06 (d, J=8.4 Hz, 1H), 8.02 (d, J=1.6, 8.4 Hz, 1H), 7.88 (d, J=8.4 Hz, 2H), 7.49 (d, J=8.4 Hz, 2H), 4.97 (dd, J=6.0, 8.4 Hz, 2H), 4.66 (dd, J=6.0, 6.8 Hz, 2H), 4.30-4.28 (m, 1H), 3.33-3.26 (m, 2H), 2.50 2.42 (m, 6H), 1.69-1.67 (m, 2H), 0.96 (t, J=7.2 Hz, 6H). m/z: [ESI⁺] 463 (M+H)⁺, (C₂₆H₃₀N₄O₂S).

### 2-([1,1′-biphenyl]-3-yl)-N-(3-(diethylamino)propyl)benzo[d]imidazo[2,1-b]thiazole-7-carboxamide (Compound 153)

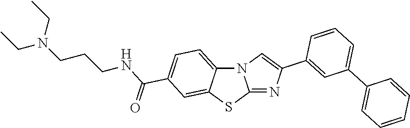 [SMILES: CCN(CC)CCCNC(=O)c1ccc2c(c1)sc1nc(-c3cccc(-c4ccccc4)c3)cn12]

Starting from 2-(3-bromophenyl)-N-(3-(diethylamino)propyl)benzo[d]imidazo[2,1-b]thiazole-7-carboxamide (200 mg, 0.412 mmol). Yield 17 mg (9%), white solid.

¹H NMR (400 MHz, DMSO) δ 8.96 (s, 1H), 8.66 (t, J=5.6 Hz, 1H), 8.50 (s, 1H), 8.17 (dd, J=1.6, 2.0 Hz, 1H), 8.05 (s, 2H), 7.89 (dd, J=1.6, 7.6 Hz, 1H), 7.75-7.72 (m, 2H), 7.62 (dd, J=1.6, 7.6 Hz, 1H), 7.58-7.50 (m, 3H), 7.46-7.38 (m, 1H), 3.33-3.26 (m, 2H), 2.59-2.51 (m, 6H), 1.73-1.71 (m, 2H), 1.01 (t, J=7.2 Hz, 6H). m/z: [ESI⁺] 483 (M+H)⁺, (C₂₉H₃₀N₄OS).

### N-(3-(diethylamino)propyl)-2-(4-(methylamino)phenyl)benzo[d]imidazo[2,1-b]thiazole-7-carboxamide (Compound 164)

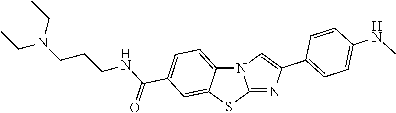 [SMILES: CCN(CC)CCCNC(=O)c1ccc2c(c1)sc1nc(-c3ccc(NC)cc3)cn12]

Starting from 2-bromo-N-(3-(diethylamino)propyl)benzo[d]imidazo[2,1-b]thiazole-7-carboxamide (300 mg, 0.733 mmol). Yield 12 mg (4%), as an off-white solid.

¹H NMR (400 MHz, DMSO) δ 8.60 (t, J=5.6 Hz, 1H), 8.51 (s, 1H), 8.45 (d, J=1.2 Hz, 1H), 7.99 (s, 2H), 7.61 (d, J=8.6 Hz, 2H), 6.60 (d, J=8.6 Hz, 2H), 5.81 (q, J=5.0 Hz, 1H), 3.33-3.26 (m, 2H), 2.72 (d, J=5.0 Hz, 3H), 2.58-2.50 (m, 6H), 1.68-1.66 (m, 2H), 0.96 (t, J=7.2 Hz, 6H). m/z: [ESI⁺] 436 (M+H)⁺, (C₂₄H₂₉N₅OS).

### N-(3-(diethylamino)propyl)-2-(4-((dimethylamino)methyl)phenyl)benzo[d]imidazo[2,1-b]thiazole-7-carboxamide (Compound 276)

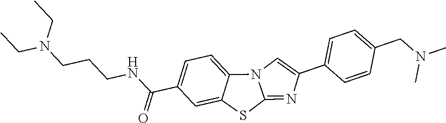 [SMILES: CCN(CC)CCCNC(=O)c1ccc2c(c1)sc1nc(-c3ccc(CN(C)C)cc3)cn12]

Starting from 2-bromo-N-(3-(diethylamino)propyl)benzo[d]imidazo[2,1-b]thiazole-7-carboxamide (300 mg, 0.733 mmol). Yield 69 mg (20%), white solid.

¹H NMR (400 MHz, DMSO) δ 8.78 (s, 1H), 8.62 (t, J=5.6 Hz, 1H), 8.48 (d, J=1.6 Hz, 1H), 8.05 (d, J=8.4 Hz, 1H), 8.01 (dd, J=1.6, 8.4 Hz, 1H), 7.82 (d, J=8.0 Hz, 2H), 7.35 (d, J=8.0 Hz, 2H), 3.40 (s, 2H), 3.32 (q, J=6.6 Hz, 2H), 2.50-2.46 (m, 6H), 2.16 (s, 6H), 1.68-1.66 (m, 2H), 0.95 (t, J=7.2 Hz, 6H). m/z: [ESI⁺] 464 (M+H)⁺, (C₂₆H₃₃N₅OS).

### N-(3-(diethylamino)propyl)-2-(4-(hydroxymethyl)phenyl)benzo[d]imidazo[2,1-b]thiazole-7-carboxamide formate (Compound 277)

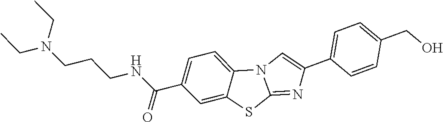 [SMILES: CCN(CC)CCCNC(=O)c1ccc2c(c1)sc1nc(-c3ccc(CO)cc3)cn12]

Starting from 2-bromo-N-(3-(diethylamino)propyl)benzo[d]imidazo[2,1-b]thiazole-7-carboxamide (300 mg, 0.733 mmol). Yield 127 mg (36%), as an off-white solid.

¹H NMR (400 MHz, DMSO) δ 8.79 (s, 1H), 8.67 (t, J=5.6 Hz, 1H), 8.49 (s, 1H), 8.26 (s, 1H), 8.06 (d, J=8.4 Hz, 1H), 8.02 (dd, J=1.6, 8.4 Hz, 1H), 7.84 (d, J=8.0 Hz, 2H), 7.39 (d, J=8.0 Hz, 2H), 5.20 (br s, 1H), 4.53 (s, 2H), 3.34 (d, J=6.4 Hz, 2H), 2.73-2.53 (m, 6H), 1.75-1.73 (m, 2H), 1.02 (t, J=7.2 Hz, 6H). m/z: [ESI⁺] 437 (M+H)⁺, (C₂₄H₂₈N₄O₂S).

### General Procedure B

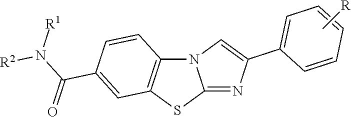

To a stirred solution of the corresponding bromide (1.00 eq.) in DMF (1M) were added amine (3.00 eq.), 9,9-dimethyl-4,5-bis(diphenylphosphino)xanthene (XantPhos) (0.10 eq.) and tris(dibenzylideneacetone)dipalladium(0) (0.20 eq.) at room temperature under a nitrogen atmosphere. The resulting mixture was stirred for 3 h at 110° C. under a carbon monoxide atmosphere (balloon). After cooling down to room temperature, the resulting mixture was purified by reverse phase flash chromatography with the addition of NH₄HCO₃ to produce the parent product while with the addition of formic acid, to produce the formate form. The fractions containing desired product were collected and concentrated under reduced pressure to afford the corresponding compounds.

### Analytical Data for Compounds Prepared According to the Methods Described Above

### N-(3-(diethylamino)propyl)-2-(o-tolyl)benzo[d]imidazo[2,1-b]thiazole-7-carboxamide (Compound 123)

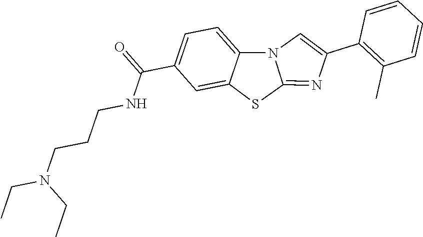 [SMILES: CCN(CC)CCCNC(=O)c1ccc2c(c1)sc1nc(-c3ccccc3C)cn12]

Starting from 7-bromo-2-(o-tolyl)benzo[d]imidazo[2,1-b]thiazole (200 mg, 0.583 mmol). Yield 38 mg (16%), white solid.

¹H NMR (400 MHz, DMSO) δ 8.63 (t, J=5.4 Hz, 1H), 8.59 (s, 1H), 8.49 (d, J=1.6 Hz, 1H), 8.19 (d, J=8.4 Hz, 1H), 8.02 (dd, J=1.6, 8.4 Hz, 1H), 7.91 (dd, J=1.6, 7.6 Hz, 1H), 7.33-7.22 (m, 3H), 3.33-3.30 (m, 2H), 2.56 (s, 3H), 2.46 (m, 6H), 1.69-1.67 (m, 2H), 0.96 (t, J=7.2 Hz, 6H). m/z: [ESI⁺] 421 (M+H)⁺, (C₂₄H₂₈N₄OS).

### N-(3-(diethylamino)propyl)-2-(4-isopropylphenyl)benzo[d]imidazo[2,1-b]thiazole-7-carboxamide formate (Compound 136)

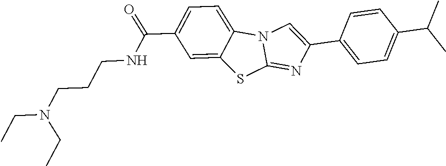 [SMILES: CCN(CC)CCCNC(=O)c1ccc2c(c1)sc1nc(-c3ccc(C(C)C)cc3)cn12]

Starting from 7-bromo-2-(4-isopropylphenyl)benzo[d]imidazo[2,1-b]thiazole (500 mg, 1.347 mmol). Yield 95 mg (14%), yellow solid. ¹H NMR (400 MHz, DMSO) δ 8.76 (s, 1H), 8.68 (t, J=5.4 Hz, 1H), 8.49 (s, 1H), 8.29 (s, 1H), 8.10-7.98 (m, 2H), 7.79 (d, J=8.4 Hz, 2H), 7.32 (d, J=8.4 Hz, 2H), 3.34 (q, J=6.4 Hz, 2H), 2.96-2.94 (m, 1H), 2.62 (m, 6H), 1.79-1.69 (m, 2H), 1.24 (d, J=6.8 Hz, 6H), 1.04 (t, J=7.2 Hz, 6H). m/z: [ESI⁺] 449 (M+H)⁺, (C₂₆H₃₂N₄OS).

### N-(3-(diethylamino)propyl)-2-(2-fluorophenyl)benzo[d]imidazo[2,1-b]thiazole-7-carboxamide (Compound 129)

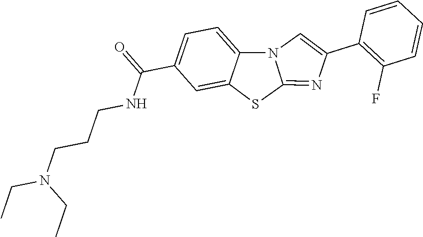 [SMILES: CCN(CC)CCCNC(=O)c1ccc2c(c1)sc1nc(-c3ccccc3F)cn12]

Starting from 7-bromo-2-(2-fluorophenyl)benzo[d]imidazo[2,1-b]thiazole (200 mg, 0.576 mmol). Yield 38 mg (16%), white solid. ¹H NMR (400 MHz, DMSO) δ 8.73 (d, J=3.6 Hz, 1H), 8.63 (t, J=5.4 Hz, 1H), 8.49 (d, J=1.6 Hz, 1H), 8.28 (d, J=8.4 Hz, 1H), 8.17 (dd, J=2.0, 8.0 Hz, 1H), 8.01 (dd, J=8.4, 1.6 Hz, 1H), 7.39-7.29 (m, 3H), 3.33-3.31 (m, 2H), 2.51-2.44 (m, 6H), 1.73-1.66 (m, 2H), 0.97 (t, J=7.2 Hz, 6H). m/z: [ESI⁺] 425 (M+H)⁺, (C₂₃H₂₅FN₄OS).

### N-(3-(diethylamino)propyl)-2-(3-fluorophenyl)benzo[d]imidazo[2,1-b]thiazole-7-carboxamide (Compound 130)

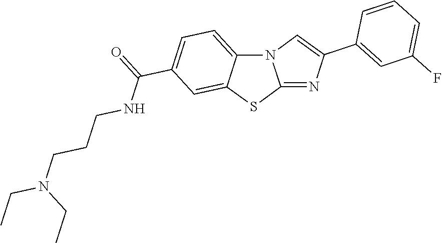 [SMILES: CCN(CC)CCCNC(=O)c1ccc2c(c1)sc1nc(-c3cccc(F)c3)cn12]

Starting from 7-bromo-2-(3-fluorophenyl)benzo[d]imidazo[2,1-b]thiazole (200 mg, 0.576 mmol). Yield 54 mg (22%), yellow solid. ¹H NMR (400 MHz, DMSO) δ 8.91 (s, 1H), 8.64 (t, J=5.4 Hz, 1H), 8.49 (d, J=1.2 Hz, 1H), 8.03 (s, 2H), 7.72 (d, J=7.8 Hz, 1H), 7.68-7.62 (m, 1H), 7.50 (dd, J=5.8, 7.8 Hz, 1H), 7.14 (dd, J=2.8, 8.6 Hz, 1H), 3.32-3.30 (m, 2H), 2.52-2.46 (m, 6H), 1.72-1.66 (m, 2H), 0.97 (t, J=7.2 Hz, 6H). m/z: [ESI⁺] 425 (M+H)⁺, (C₂₃H₂₅FN₄OS).

### 2-(3-chlorophenyl)-N-(3-(diethylamino)propyl)benzo[d]imidazo[2,1-b]thiazole-7-carboxamide (Compound 131)

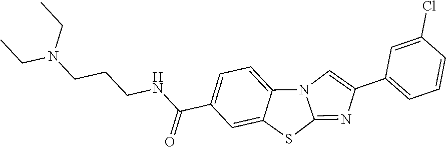 [SMILES: CCN(CC)CCCNC(=O)c1ccc2c(c1)sc1nc(-c3cccc(Cl)c3)cn12]

Starting from 7-bromo-2-(3-chlorophenyl)benzo[d]imidazo[2,1-b]thiazole (200 mg, 0.550 mmol). Yield 39 mg (16%), light yellow solid. ¹H NMR (400 MHz, DMSO) δ 8.94 (s, 1H), 8.63 (t, J=5.4 Hz, 1H), 8.49 (d, J=1.2 Hz, 1H), 8.03 (s, 2H), 7.92 (d, J=1.8 Hz, 1H), 7.87-7.80 (m, 1H), 7.49 (dd, J=1.6, 8.0 Hz, 1H), 7.37 (dd, J=2.2, 8.0 Hz, 1H), 3.33-3.26 (m, 2H), 2.51-2.46 (m, 6H), 1.68-1.66 (m, 2H), 0.96 (t, J=7.2 Hz, 6H). m/z: [ESI⁺] 441, 443 (M+H)⁺, (C₂₃H₂₅ClN₄OS).

### 2-(4-chlorophenyl)-N-(3-(piperidin-1-yl)propyl)benzo[d]imidazo[2,1-b]thiazole-7-carboxamide (Compound 132)

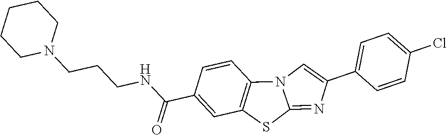 [SMILES: O=C(NCCCN1CCCCC1)c1ccc2c(c1)sc1nc(-c3ccc(Cl)cc3)cn12]

Starting from 7-bromo-2-(4-chlorophenyl)benzo[d]imidazo[2,1-b]thiazole (200 mg, 0.550 mmol). Yield 38 mg (15%), white solid. ¹H NMR (400 MHz, DMSO) δ 8.88 (s, 1H), 8.65 (t, J=5.4 Hz, 1H), 8.49 (d, J=1.6 Hz, 1H), 8.05 (d, J=8.4 Hz, 1H), 8.02 (dd, J=1.6, 8.4 Hz, 1H), 7.89 (d, J=8.6 Hz, 2H), 7.52 (d, J=8.6 Hz, 2H), 3.33-3.26 (m, 2H), 2.38-2.30 (m, 6H), 1.72-1.70 (m, 2H), 1.53-1.48 (m, 4H), 1.42-1.35 (m, 2H). m/z: [ESI⁺] 453, 455 (M+H)⁺, (C₂₄H₂₅ClN₄OS).

### 2-(2-chlorophenyl)-N-(3-(diethylamino)propyl)benzo[d]imidazo[2,1-b]thiazole-7-carboxamide (Compound 124)

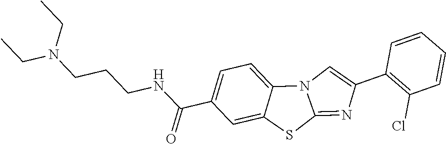 [SMILES: CCN(CC)CCCNC(=O)c1ccc2c(c1)sc1nc(-c3ccccc3Cl)cn12]

Starting from 7-bromo-2-(2-chlorophenyl)benzo[d]imidazo[2,1-b]thiazole (400 mg, 1.100 mmol). Yield 87 mg (18%), white solid. ¹H NMR (400 MHz, DMSO) δ 8.99 (s, 1H), 8.64 (t, J=5.4 Hz, 1H), 8.50 (d, J=1.6 Hz, 1H), 8.30 (d, J=8.4 Hz, 1H), 8.21 (dd, J=1.8, 8.0 Hz, 1H), 8.02 (dd, J=1.6, 8.4 Hz, 1H), 7.57 (dd, J=1.2, 8.0 Hz, 1H), 7.46 (dd, J=1.2, 7.6 Hz, 1H), 7.35 (dd, J=1.8, 7.6 Hz, 1H), 3.33-3.26 (m, 2H), 2.50-2.46 (m, 6H), 1.69-1.67 (m, 2H), 0.96 (t, J=7.2 Hz, 6H). m/z: [ESI⁺]441, 443 (M+H)⁺, (C₂₃H₂₅ClN₄OS).

### N-(3-(diethylamino)propyl)-2-(4-(methylcarbamoyl)phenyl)benzo[d]imidazo[2,1-b]thiazole-7-carboxamide (Compound 125)

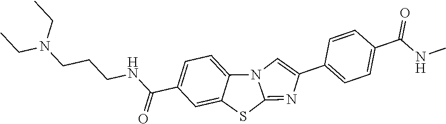 [SMILES: CCN(CC)CCCNC(=O)c1ccc2c(c1)sc1nc(-c3ccc(C(=O)NC)cc3)cn12]

Starting from 4-(7-bromobenzo[d]imidazo[2,1-b]thiazol-2-yl)-N-methylbenzamide (200 mg, 0.518 mmol). Yield 47 mg (20%), white solid. 1H NMR (400 MHz, DMSO) δ 8.94 (s, 1H), 8.66 (t, J=5.4 Hz, 1H), 8.50 (d, J=1.6 Hz, 1H), 8.47-8.45 (m, 1H), 8.07 (d, J=8.4 Hz, 1H), 8.03 (dd, J=1.6, 8.4 Hz, 1H), 7.96 (d, J=8.6 Hz, 1H), 7.92 (d, J=8.6 Hz, 1H), 3.33-3.26 (m, 2H), 2.81 (d, J=4.4 Hz, 3H), 2.51-2.46 (m, 6H), 1.78-1.68 (m, 2H), 1.01 (t, J=7.2 Hz, 6H). m/z: [ESI⁺] 464 (M+H)⁺, (C₂₅H₂₉N₅O₂S).

### N-(3-(diethylamino)propyl)-2-(4-ethylphenyl)benzo[d]imidazo[2,1-b]thiazole-7-carboxamide (Compound 126)

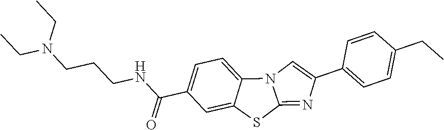 [SMILES: CCc1ccc(-c2cn3c(n2)sc2cc(C(=O)NCCCN(CC)CC)ccc23)cc1]

Starting from 7-bromo-2-(4-ethylphenyl)benzo[d]imidazo[2,1-b]thiazole (200 mg, 0.560 mmol). Yield 30 mg (12%), light yellow solid. ¹H NMR (400 MHz, DMSO) δ 8.76 (s, 1H), 8.62 (t, J=5.4 Hz, 1H), 8.48 (s, 1H), 8.05 (d, J=8.4 Hz, 1H), 8.02 (dd, J=1.6, 8.4 Hz, 1H), 7.79 (d, J=7.8 Hz, 2H), 7.29 (d, J=7.8 Hz, 2H), 3.33-3.26 (m, 2H), 2.64 (q, J=7.6 Hz, 2H), 2.50-2.46 (m, 6H), 1.68-1.66 (m, 2H), 1.22 (t, J=7.6 Hz, 3H), 0.96 (t, J=7.2 Hz, 6H). m/z: [ESI⁺] 435 (M+H)⁺, (C₂₅H₃₀N₄OS).

### N-(3-(4,4-difluoropiperidin-1-yl)propyl)-2-(m-tolyl)benzo[d]imidazo[2,1-b]thiazole-7-carboxamide (Compound 133)

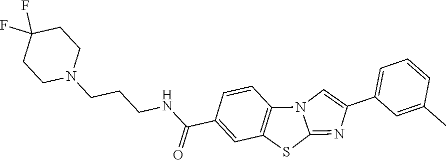 [SMILES: Cc1cccc(-c2cn3c(n2)sc2cc(C(=O)NCCCN4CCC(F)(F)CC4)ccc23)c1]

Starting from 7-bromo-2-(m-tolyl)benzo[d]imidazo[2,1-b]thiazole (200 mg, 0.583 mmol). Yield 45 mg (16%), white solid. ¹H NMR (400 MHz, DMSO) δ 8.81 (s, 1H), 8.60 (t, J=5.4 Hz, 1H), 8.49 (d, J=1.6 Hz, 1H), 8.06 (d, J=8.4 Hz, 1H), 8.02 (dd, J=1.6, 8.4 Hz, 1H), 7.72 (d, J=1.8 Hz, 1H), 7.67 (d, J=7.6 Hz, 1H), 7.34 (d, J=7.6 Hz, 1H), 7.15-7.11 (m, 1H), 3.38-3.34 (m, 2H), 2.53-2.48 (m, 4H), 2.43 (t, J=7.2 Hz, 2H), 2.38 (s, 3H), 2.00-1.89 (m, 4H), 1.73-1.71 (m, 2H). m/z: [ESI⁺] 469 (M+H)⁺, (C₂₅H₂₆F₂N₄OS).

### N-(3-morpholinopropyl)-2-(m-tolyl)benzo[d]imidazo[2,1-b]thiazole-7-carboxamide (Compound 134)

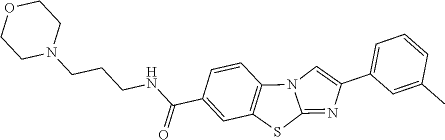 [SMILES: Cc1cccc(-c2cn3c(n2)sc2cc(C(=O)NCCCN4CCOCC4)ccc23)c1]

Starting from 7-bromo-2-(m-tolyl)benzo[d]imidazo[2,1-b]thiazole (200 mg, 0.583 mmol). Yield 51 mg (20%), as an off-white solid. ¹H NMR (400 MHz, DMSO) δ 8.81 (s, 1H), 8.61 (t, J=5.4 Hz, 1H), 8.48 (d, J=1.6 Hz, 1H), 8.06 (d, J=8.4 Hz, 1H), 8.02 (dd, J=1.6, 8.4 Hz, 1H), 7.72 (s, 1H), 7.67 (dd, J=1.6, 7.6 Hz, 1H), 7.34 (dd, J=1.6, 7.6 Hz, 1H), 7.13 (d, J=7.6 Hz, 1H), 3.58 (t, J=4.6 Hz, 4H), 3.33-3.26 (m, 2H), 2.53-2.48 (m, 4H), 2.38 (s, 3H), 2.38-2.36 (m, 2H), 1.72 (t, J=7.2 Hz, 2H). m/z: [ESI⁺] 435 (M+H)⁺, (C₂₄H₂₆N₄O₂S).

### N-(3-(4-fluoropiperidin-1-yl)propyl)-2-(m-tolyl)benzo[d]imidazo[2,1-b]thiazole-7-carboxamide (Compound 137)

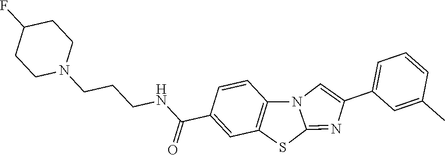 [SMILES: Cc1cccc(-c2cn3c(n2)sc2cc(C(=O)NCCCN4CCC(F)CC4)ccc23)c1]

Starting from 7-bromo-2-(m-tolyl)benzo[d]imidazo[2,1-b]thiazole (200 mg, 0.583 mmol). Yield 30 mg (11%), white solid. ¹H NMR (400 MHz, DMSO) δ 8.81 (s, 1H), 8.61 (t, J=5.4 Hz, 1H), 8.48 (d, J=1.6 Hz, 1H), 8.05 (d, J=8.4 Hz, 1H), 8.02 (dd, J=1.6, 8.4 Hz, 1H), 7.72 (s, 1H), 7.67 (d, J=7.6 Hz, 1H), 7.34 (dd, J=1.6, 7.6 Hz, 1H), 7.13 (d, J=7.6 Hz, 1H), 4.68 (d, J=47.8 Hz, 1H), 3.33-3.26 (m, 2H), 2.55-2.48 (m, 4H), 2.37 (s, 3H), 2.32-2.25 (m, 2H), 1.91-1.77 (m, 2H), 1.75-1.68 (m, 4H). m/z: [ESI⁺] 451 (M+H)⁺, (C₂₅H₂₇FN₄OS).

### N-(3-(1,1-dioxidothiomorpholino)propyl)-2-(m-tolyl)benzo[d]imidazo[2,1-b]thiazole-7-carboxamide (Compound 135)

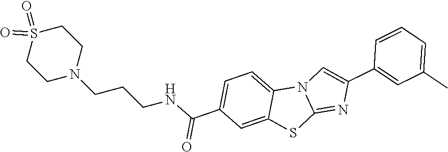 [SMILES: Cc1cccc(-c2cn3c(n2)sc2cc(C(=O)NCCCN4CCS(=O)(=O)CC4)ccc23)c1]

Starting from 7-bromo-2-(m-tolyl)benzo[d]imidazo[2,1-b]thiazole (200 mg, 0.583 mmol). Yield 55 mg (20%), white solid. ¹H NMR (400 MHz, DMSO) δ 8.81 (s, 1H), 8.58 (t, J=5.4 Hz, 1H), 8.49 (d, J=1.6 Hz, 1H), 8.06 (d, J=8.4 Hz, 1H), 8.02 (dd, J=1.6, 8.4 Hz, 1H), 7.72 (d, J=2.0 Hz, 1H), 7.67 (d, J=7.8 Hz, 1H), 7.34 (t, J=1.6, 7.6 Hz, 1H), 7.16-7.09 (m, 1H), 3.36-3.31 (m, 2H), 3.09 (t, J=5.2 Hz, 4H), 2.94-2.87 (m, 4H), 2.56 (t, J=7.2 Hz, 2H), 2.38 (s, 3H), 1.72-1.70 (m, 2H). m/z: [ESI⁺] 483 (M+H)⁺, (C₂₄H₂₆N₄O₃S₂).

### N-(3-(tetrahydro-2H-pyran-4-yl)propyl)-2-(m-tolyl)benzo[d]imidazo[2,1-b]thiazole-7-carboxamide (Compound 139)

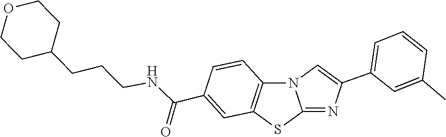 [SMILES: Cc1cccc(-c2cn3c(n2)sc2cc(C(=O)NCCCC4CCOCC4)ccc23)c1]

Starting from 7-bromo-2-(m-tolyl)benzo[d]imidazo[2,1-b]thiazole (200 mg, 0.583 mmol). Yield 35 mg (14%), white solid. ¹H NMR (400 MHz, DMSO) δ 8.81 (s, 1H), 8.59 (t, J=5.4 Hz, 1H), 8.49 (d, J=1.6 Hz, 1H), 8.05 (d, J=8.4 Hz, 1H), 8.03 (dd, J=1.6, 8.4 Hz, 1H), 7.72 (s, 1H), 7.67 (d, J=7.6 Hz, 1H), 7.34 (dd, J=1.6, 7.6 Hz, 1H), 7.13 (d, J=7.6 Hz, 1H), 3.86-3.81 (m, 2H), 3.33-3.23 (m, 4H), 2.38 (s, 3H), 1.62-1.44 (m, 5H), 1.28 (q, J=7.2 Hz, 2H), 1.15 (dq, J=4.2, 12.0 Hz, 2H). m/z: [ESI⁺] 434 (M+H)⁺, (C₂₅H₂₇N₃O₂S).

### N-(3-(diethylamino)propyl)-2-(4-methoxyphenyl)benzo[d]imidazo[2,1-b]thiazole-7-carboxamide (Compound 142)

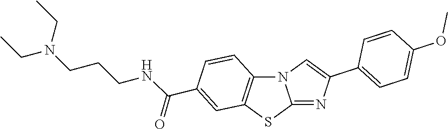 [SMILES: CCN(CC)CCCNC(=O)c1ccc2c(c1)sc1nc(-c3ccc(OC)cc3)cn12]

Starting from 7-bromo-2-(4-methoxyphenyl)benzo[d]imidazo[2,1-b]thiazole (600 mg, 1.670 mmol). Yield 91 mg (12%), as an off-white solid. ¹H NMR (400 MHz, DMSO) δ 8.69 (s, 1H), 8.62 (t, J=5.4 Hz, 1H), 8.47 (d, J=1.6 Hz, 1H), 8.03 (d, J=8.4 Hz, 1H), 8.00 (d, J=1.6, 8.4 Hz, 1H), 7.80 (d, J=8.6 Hz, 2H), 7.02 (d, J=8.6 Hz, 2H), 3.80 (s, 3H), 3.32-3.26 (m, 2H), 2.50-2.46 (m, 6H), 1.68-1.66 (m, 2H), 0.95 (t, J=7.2 Hz, 6H). m/z: [ESI⁺] 437 (M+H)⁺, (C₂₄H₂₈N₄O₂S).

### General Procedure C for Amide Formation

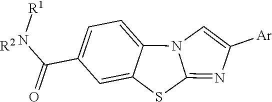

To a stirred solution of the corresponding carboxylic acid (1.00 eq.) in DMF (0.30M) were added HATU (1.30 eq.), amine (1.20 eq.) and DIPEA (3.00 eq.) at room temperature under a nitrogen atmosphere. The resulting mixture was stirred for 1 h at room temperature under a nitrogen atmosphere. The resulting mixture was purified by reverse phase flash chromatography with the addition of NH₄HCO₃ will produce the parent product while with the addition of formic acid, will produce the product as formate form. The fractions containing desired product were collected, concentrated under reduced pressure and lyophilized to produce the corresponding products.

### N-(3-(diethylamino)propyl)-2-(m-tolyl)benzo[d]imidazo[2,1-b]thiazole-7-carboxamide (Compound 118)

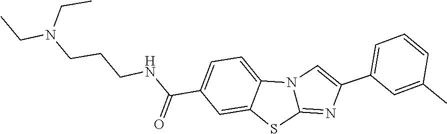 [SMILES: CCN(CC)CCCNC(=O)c1ccc2c(c1)sc1nc(-c3cccc(C)c3)cn12]

Starting from 2-(m-tolyl)benzo[d]imidazo[2,1-b]thiazole-7-carboxylic acid (100 mg, 0.324 mmol); Yield 45 mg (33%), as a white solid. ¹H NMR (400 MHz, DMSO) δ 8.75 (s, 1H), 8.61 (t, J=5.2 Hz, 1H), 8.47 (d, J=1.2 Hz, 1H), 8.05-7.99 (m, 2H), 7.77 (d, J=8.0 Hz, 2H), 7.26 (d, J=8.0 Hz, 2H), 3.34-3.30 (m, 2H), 2.34 (s, 3H), 2.51-2.46 (m, 6H), 1.71-1.64 (m, 2H), 0.96 (t, J=7.2 Hz, 6H). m/z: [ESI⁺] 421 (M+H)⁺, (C₂₄H₂₈N₄OS).

### N-((1r,3r)-3-(piperidin-1-yl)cyclobutyl)-2-(m-tolyl)benzo[d]imidazo[2,1-b]thiazole-7-carboxamide (Compound 151)

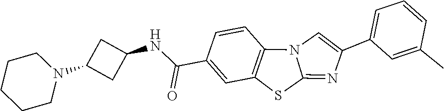 [SMILES: Cc1cccc(-c2cn3c(n2)sc2cc(C(=O)N[C@H]4C[C@H](N5CCCCC5)C4)ccc23)c1]

Starting from 2-(m-tolyl)benzo[d]imidazo[2,1-b]thiazole-7-carboxylic acid (700 mg, 2.270 mmol); Yield 163 mg (16%), as an off-white solid. ¹H NMR (400 MHz, DMSO) δ 8.81 (s, 1H), 8.79 (d, J=6.8 Hz, 1H), 8.51 (s, 1H), 8.06 (s, 2H), 7.72 (s, 1H), 7.67 (d, J=7.6 Hz, 1H), 7.33 (dd, J=1.6, 7.6 Hz, 1H), 7.13 (d, J=7.6 Hz, 1H), 4.32 (d, J=6.8 Hz, 1H), 2.87 (s, 1H), 2.38 (s, 3H), 2.30-2.20 (m, 6H), 2.18-2.06 (m, 2H), 1.57-1.50 (m, 4H), 1.48-1.38 (m, 2H). m/z: [ESI⁺] 445 (M+H)⁺, (C₂₆H₂₈N₄OS).

### 2-(4-chlorophenyl)-N-(3-(diethylamino)propyl)benzo[d]imidazo[2,1-b]thiazole-7-carboxamide (Compound 111)

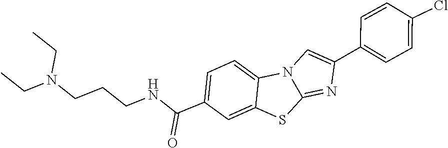 [SMILES: CCN(CC)CCCNC(=O)c1ccc2c(c1)sc1nc(-c3ccc(Cl)cc3)cn12]

Starting from 2-(4-chlorophenyl)benzo[d]imidazo[2,1-b]thiazole-7-carboxylic acid (150 mg, 0.456 mmol); Yield 137 mg (68%), as a white solid. ¹H NMR (400 MHz, DMSO) δ 8.87 (s, 1H), 8.62 (dd, J=1.6, 5.6 Hz, 1H), 8.49 (d, J=1.6 Hz, 1H), 8.05 (d, J=8.4 Hz, 1H), 8.02 (dd, J=1.6, 8.4 Hz, 1H), 7.89 (d, J=8.4 Hz, 2H), 7.52 (d, J=8.4 Hz, 2H), 3.31-3.26 (m, 2H), 2.50-2.46 (m, 6H), 1.68 (p, J=7.2 Hz, 2H), 0.96 (t, J=7.2 Hz, 6H). m/z: [ESI⁺] 441, 443 (M+H)⁺, (C₂₃H₂₅ClN₄OS).

### N-(3-(azepan-1-yl)propyl)-2-phenylbenzo[d]imidazo[2,1-b]thiazole-7-carboxamide (Compound 103)

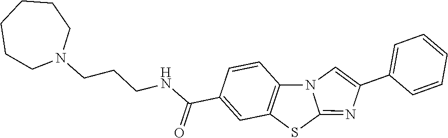 [SMILES: O=C(NCCCN1CCCCCC1)c1ccc2c(c1)sc1nc(-c3ccccc3)cn12]

Starting from 2-phenylbenzo[d]imidazo[2,1-b]thiazole-7-carboxylic acid (200 mg, 0.680 mmol); Yield 72 mg (24%), as a white solid. ¹H NMR (400 MHz, DMSO) δ 8.83 (s, 1H), 8.60 (t, J=5.4 Hz, 1H), 8.49 (d, J=1.6 Hz, 1H), 8.07 (d, J=8.4 Hz, 1H), 8.02 (dd, J=8.4, 1.6 Hz, 1H), 7.89 (d, J=7.6 Hz, 2H), 7.46 (dd, J=1.6, 7.6 Hz, 2H), 7.31 (dd, J=1.6, 7.4 Hz, 1H), 3.33-3.26 (m, 2H), 2.59 (t, J=5.4 Hz, 4H), 2.52-2.49 (m, 2H), 1.69 (p, J=7.0 Hz, 2H), 1.63-1.51 (m, 8H). m/z: [ESI⁺] 433 (M+H)⁺, (C₂₅H₂₈N₄OS).

### 2-phenyl-N-(3-(piperidin-1-yl)propyl)benzo[d]imidazo[2,1-b]thiazole-7-carboxamide (Compound 101)

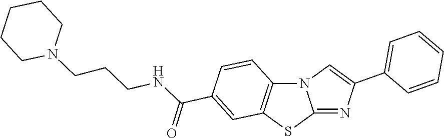 [SMILES: O=C(NCCCN1CCCCC1)c1ccc2c(c1)sc1nc(-c3ccccc3)cn12]

Starting from 2-phenylbenzo[d]imidazo[2,1-b]thiazole-7-carboxylic acid (200 mg, 0.680 mmol); Yield 120 mg (42%), as a white solid. ¹H NMR (400 MHz, DMSO) δ 8.83 (s, 1H), 8.64 (t, J=5.4 Hz, 1H), 8.49 (d, J=1.6 Hz, 1H), 8.07 (d, J=8.4 Hz, 1H), 8.02 (dd, J=1.6, 8.4 Hz, 1H), 7.88 (d, J=7.8 Hz, 2H), 7.45 (dd, J=1.6, 7.6 Hz, 2H), 7.31 (dd, J=1.6, 7.4 Hz, 1H), 3.34-3.26 (m, 2H), 2.36 2.31 (m, 6H), 1.71-1.69 (m, 2H), 1.51-1.49 (m, 4H), 1.42-1.36 (m, 2H). m/z: [ESI⁺] 419 (M+H)⁺, (C₂₄H₂₆N₄OS).

### (S)—N-((1-ethylpyrrolidin-2-yl)methyl)-2-(3-methoxyphenyl)benzo[d]imidazo[2,1-b]thiazole-7-carboxamide (Compound 119)

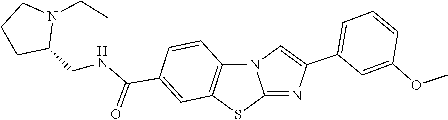 [SMILES: CCN1CCC[C@H]1CNC(=O)c1ccc2c(c1)sc1nc(-c3cccc(OC)c3)cn12]

Starting from 2-(3-methoxyphenyl)benzo[d]imidazo[2,1-b]thiazole-7-carboxylic acid (150 mg, 0.462 mmol); Yield 35 mg (17%), as a white solid. ¹H NMR (400 MHz, DMSO) δ 8.84 (s, 1H), 8.51 (t, J=5.8 Hz, 1H), 8.49 (d, J=1.6 Hz, 1H), 8.07-8.01 (m, 2H), 7.50-7.43 (m, 2H), 7.40-7.32 (m, 1H), 6.92-6.85 (m, 1H), 3.83 (s, 3H), 3.50-3.44 (m, 1H), 3.16-3.03 (m, 2H), 2.88 (td, J=7.2, 12.0 Hz, 1H), 2.66-2.57 (m, 1H), 2.39-2.25 (m, 1H), 2.21-2.12 (m, 1H), 1.88-1.77 (m, 1H), 1.73-1.57 (m, 3H), 1.07 (t, J=7.2 Hz, 3H). m/z: [ESI⁺] 435 (M+H)⁺, (C₂₄H₂₆N₄O₂S).

### (R)—N-((1-ethylpyrrolidin-2-yl)methyl)-2-(3-methoxyphenyl)benzo[d]imidazo[2,1-b]thiazole-7-carboxamide (Compound 127)

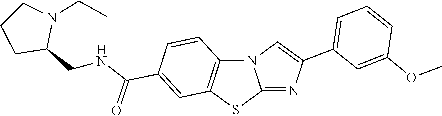 [SMILES: CCN1CCC[C@@H]1CNC(=O)c1ccc2c(c1)sc1nc(-c3cccc(OC)c3)cn12]

Starting from 2-(3-methoxyphenyl)benzo[d]imidazo[2,1-b]thiazole-7-carboxylic acid (150 mg, 0.462 mmol); Yield 69 mg (34%), as a white solid. ¹H NMR (400 MHz, DMSO) δ 8.84 (s, 1H), 8.52-8.47 (m, 2H), 8.06-8.01 (m, 2H), 7.48-7.45 (m, 2H), 7.40-7.32 (m, 1H), 6.93-6.80 (m, 1H), 3.84 (s, 3H), 3.50-3.43 (m, 1H), 3.17-3.01 (m, 2H), 2.90-2.83 (m, 1H), 2.65-2.58 (m, 1H), 2.35-2.24 (m, 1H), 2.15 (q, J=8.4 Hz, 1H), 1.90-1.77 (m, 1H), 1.73-1.56 (m, 3H), 1.07 (t, J=7.2 Hz, 3H). m/z: [ESI⁺]435 (M+H)⁺, (C₂₄H₂₆N₄O₂S).

### 2-(3-cyanophenyl)-N-(3-(diethylamino)propyl)benzo[d]imidazo[2,1-b]thiazole-7-carboxamide (Compound 145)

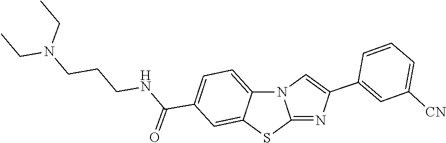 [SMILES: CCN(CC)CCCNC(=O)c1ccc2c(c1)sc1nc(-c3cccc(C#N)c3)cn12]

Starting from 2-(3-cyanophenyl)benzo[d]imidazo[2,1-b]thiazole-7-carboxylic acid (200 mg, 0.626 mmol); Yield 50 mg (18%), as a white solid. ¹H NMR (400 MHz, DMSO) δ 8.99 (s Hz, 1H), 8.64 (t, J=5.4 Hz, 1H), 8.50 (d, J=1.6 Hz, 1H), 8.27 (d, J=1.8 Hz, 1H), 8.20 (dd, J=1.8, 8.0 Hz, 1H), 8.06-8.00 (m, 2H), 7.77 (dd, J=1.6, 7.6 Hz, 1H), 7.68 (dd, J=1.2, 7.8 Hz, 1H), 3.33-3.26 (m, 2H), 2.51-2.43 (m, 6H), 1.69-1.67 (m, 2H), 0.96 (t, J=7.2 Hz, 6H). m/z: [ESI⁺] 432 (M+H)⁺, (C₂₄H₂₅N₅OS).

### N-(3-(pyrrolidin-1-yl)propyl)-2-(m-tolyl)benzo[d]imidazo[2,1-b]thiazole-7-carboxamide (Compound 149)

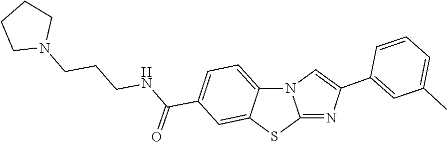 [SMILES: Cc1cccc(-c2cn3c(n2)sc2cc(C(=O)NCCCN4CCCC4)ccc23)c1]

Starting from 2-(m-tolyl)benzo[d]imidazo[2,1-b]thiazole-7-carboxylic acid (100 mg, 0.324 mmol); Yield 84 mg (62%), as a white solid. ¹H NMR (400 MHz, DMSO) δ 8.81 (s, 1H), 8.68 (t, J=5.4 Hz, 1H), 8.48 (d, J=1.6 Hz, 1H), 8.05 (d, J=8.4 Hz, 1H), 8.01 (dd, J=1.6, 8.4 Hz, 1H), 7.72 (s, 1H), 7.67 (d, J=7.8 Hz, 1H), 7.33 (dd, J=1.6, 7.6 Hz, 1H), 7.13 (d, J=7.6 Hz, 1H), 3.34-3.26 (m, 2H), 2.50-2.46 (m, 6H), 2.38 (s, 3H), 1.82-1.54 (m, 6H). m/z: [ESI⁺] 419 (M+H)⁺, (C₂₄H₂₆N₄OS).

### N-(3-(2-oxopyrrolidin-1-yl)propyl)-2-(m-tolyl)benzo[d]imidazo[2,1-b]thiazole-7-carboxamide (Compound 150)

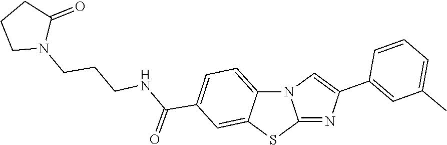 [SMILES: Cc1cccc(-c2cn3c(n2)sc2cc(C(=O)NCCCN4CCCC4=O)ccc23)c1]

Starting from 2-(m-tolyl)benzo[d]imidazo[2,1-b]thiazole-7-carboxylic acid (100 mg, 0.324 mmol); Yield 80 mg (57%), as a white solid. ¹H NMR (400 MHz, DMSO) δ 8.81 (s, 1H), 8.59 (t, J=5.6 Hz, 1H), 8.49 (d, J=1.6 Hz, 1H), 8.06 (d, J=8.4 Hz, 1H), 8.02 (dd, J=1.6, 8.4 Hz, 1H), 7.72 (s, 1H), 7.67 (d, J=7.6 Hz, 1H), 7.33 (dd, J=1.6, 7.6 Hz, 1H), 7.13 (d, J=7.6 Hz, 1H), 3.37 (t, J=7.0 Hz, 2H), 3.27 (dd, J=6.2, 8.8 Hz, 4H), 2.38 (s, 3H), 2.24 (t, J=8.0 Hz, 2H), 1.95-1.93 (m, 2H), 1.76-1.74 (m, 2H). m/z: [ESI⁺] 433 (M+H)⁺, (C₂₄H₂₄N₄O₂S).

### N-(3-(diethylamino)propyl)-2-(2-fluoro-3-methylphenyl)benzo[d]imidazo[2,1-b]thiazole-7-carboxamide (Compound 143)

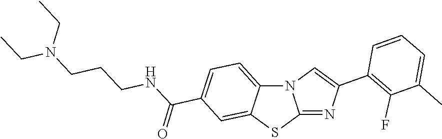 [SMILES: CCN(CC)CCCNC(=O)c1ccc2c(c1)sc1nc(-c3cccc(C)c3F)cn12]

Starting from 2-(2-fluoro-3-methylphenyl)benzo[d]imidazo[2,1-b]thiazole-7-carboxylic acid (200 mg, 0.613 mmol); Yield 24 mg (9%), as a white solid. ¹H NMR (400 MHz, DMSO) δ 8.73 (d, J=4.0 Hz, 1H), 8.63 (t, J=5.4 Hz, 1H), 8.49 (d, J=1.6 Hz, 1H), 8.26 (d, J=8.4 Hz, 1H), 8.01 (dd, J=2.0, 6.4 Hz, 1H), 7.99 (dd, J=1.6, 8.4 Hz, 1H), 7.2-7.17 (m, 2H), 3.33-3.26 (m, 2H), 2.50-2.47 (m, 6H), 2.34 (d, J=2.2 Hz, 3H), 1.69-1.67 (m, 2H), 0.97 (t, J=7.2 Hz, 6H). m/z: [ESI⁺] 439 (M+H)⁺, (C₂₄H₂₇FN₄OS).

### N-(3-(diethylamino)propyl)-2-(2-fluoro-5-methylphenyl)benzo[d]imidazo[2,1-b]thiazole-7-carboxamide (Compound 144)

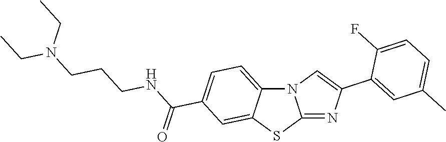 [SMILES: CCN(CC)CCCNC(=O)c1ccc2c(c1)sc1nc(-c3cc(C)ccc3F)cn12]

Starting from 2-(2-fluoro-5-methylphenyl)benzo[d]imidazo[2,1-b]thiazole-7-carboxylic acid (200 mg, 0.613 mmol); Yield 22 mg (8%), as a white solid. ¹H NMR (400 MHz, DMSO) δ 8.70 (d, J=3.6 Hz, 1H), 8.63 (t, J=5.4 Hz, 1H), 8.49 (d, J=1.6 Hz, 1H), 8.27 (d, J=8.4 Hz, 1H), 8.05-7.97 (m, 2H), 7.22 (dd, J=8.4, 11.2 Hz, 1H), 7.16-7.14 (m, 1H), 3.33-3.26 (m, 2H), 2.50-2.46 (m, 6H), 2.36 (s, 3H), 1.70-1.68 (m, 2H), 0.97 (t, J=7.2 Hz, 6H). m/z: [ESI⁺] 439 (M+H)⁺, (C₂₄H₂₇FN₄OS).

### N-(3-oxo-3-(pyrrolidin-1-yl)propyl)-2-(m-tolyl)benzo[d]imidazo[2,1-b]thiazole-7-carboxamide (Compound 159)

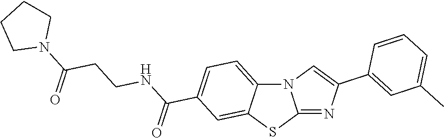 [SMILES: Cc1cccc(-c2cn3c(n2)sc2cc(C(=O)NCCC(=O)N4CCCC4)ccc23)c1]

Starting from 2-(m-tolyl)benzo[d]imidazo[2,1-b]thiazole-7-carboxylic acid (100 mg, 0.324 mmol); Yield 33 mg (24%), as a white solid. ¹H NMR (400 MHz, DMSO) δ 8.81 (s, 1H), 8.62 (t, J=5.4 Hz, 1H), 8.49 (d, J=1.6 Hz, 1H), 8.05 (d, J=8.3 Hz, 1H), 8.03 (dd, J=1.6, 8.4 Hz, 1H), 7.72 (s, 1H), 7.67 (d, J=7.6 Hz, 1H), 7.34-7.32 (m, 1H), 7.12 (d, J=7.6 Hz, 1H), 3.52 (q, J=7.0 Hz, 2H), 3.41 (t, J=6.8 Hz, 2H), 3.32-3.31 (m, 2H), 2.57 (t, J=7.2 Hz, 2H), 2.38 (s, 3H), 1.93-1.82 (m, 2H), 1.82-1.72 (m, 2H). m/z: [ESI⁺] 433 (M+H)⁺, (C₂₄H₂₄N₄O₂S).

### N-(3-(diethylamino)propyl)-2-(3-methoxyphenyl)benzo[d]imidazo[2,1-b]thiazole-7-carboxamide (Compound 152)

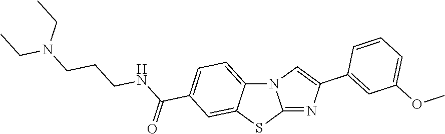 [SMILES: CCN(CC)CCCNC(=O)c1ccc2c(c1)sc1nc(-c3cccc(OC)c3)cn12]

Starting from 2-(3-methoxyphenyl)benzo[d]imidazo[2,1-b]thiazole-7-carboxylic acid (400 mg, 1.233 mmol); Yield 39 mg (7%), as a white solid. ¹H NMR (400 MHz, DMSO) δ 8.84 (s, 1H), 8.63 (t, J=5.4 Hz, 1H), 8.48 (d, J=1.6 Hz, 1H), 8.05 (d, J=8.4 Hz, 1H), 8.02 (dd, J=1.6, 8.4 Hz, 1H), 7.49-7.42 (m, 2H), 7.37-7.35 (m, 1H), 6.88 (dd, J=2.0, 8.0 Hz, 1H), 3.83 (s, 3H), 3.33-3.26 (m, 2H), 2.53-2.46 (m, 6H), 1.70-1.68 (m, 2H), 0.97 (t, J=7.2 Hz, 6H). m/z: [ESI⁺] 437 (M+H)⁺, (C₂₄H₂₈N₄O₂S).

### N-(3-(diethylamino)propyl)-2-(4-(dimethylcarbamoyl)phenyl)benzo[d]imidazo[2,1-b]thiazole-7-carboxamide formate (Compound 154)

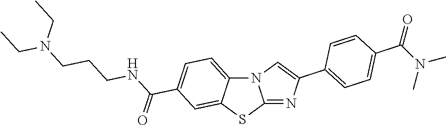 [SMILES: CCN(CC)CCCNC(=O)c1ccc2c(c1)sc1nc(-c3ccc(C(=O)N(C)C)cc3)cn12]

Starting from 4-(7-((3-(diethylamino)propyl)carbamoyl)benzo[d]imidazo[2,1-b]thiazol-2-yl)benzoic acid (120 mg, 0.266 mmol); Yield 27 mg (19%), as a white solid. ¹H NMR (400 MHz, DMSO) δ 8.91 (s, 1H), 8.67 (t, J=5.4 Hz, 1H), 8.50 (d, J=1.6 Hz, 1H), 8.11-8.00 (m, 2H), 7.92 (d, J=8.6 Hz, 2H), 7.50 (d, J=8.6 Hz, 2H), 3.33-3.26 (m, 2H), 2.99 (s, 6H), 2.65-2.56 (m, 6H), 1.79-1.67 (m, 2H), 1.01 (t, J=7.2 Hz, 6H). m/z: [ESI⁺] 478 (M+H)⁺, (C₂₆H₃₁N₅O₂S).

### 2-(4-(Methylcarbamoyl)phenyl)-N-(3-(piperidin-1-yl)propyl)benzo[d]imidazo[2,1-b]thiazole-7-carboxamide (Compound 174)

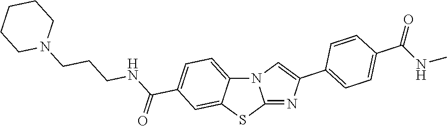 [SMILES: CNC(=O)c1ccc(-c2cn3c(n2)sc2cc(C(=O)NCCCN4CCCCC4)ccc23)cc1]

Starting from 2-(4-(methylcarbamoyl)phenyl)benzo[d]imidazo[2,1-b]thiazole-7-carboxylic acid (120 mg, 0.342 mmol); Yield 59 mg (36%), as a white solid. ¹H NMR (400 MHz, DMSO) δ 8.94 (s, 1H), 8.66 (t, J=5.4 Hz, 1H), 8.50 (d, J=1.6 Hz, 1H), 8.47-8.45 (m, 1H), 8.10-8.00 (m, 2H), 7.95 (d, J=8.4 Hz, 2H), 7.91 (d, J=8.4 Hz, 2H), 3.33-3.26 (m, 2H), 2.81 (d, J=4.4 Hz, 3H), 2.50-2.37 (m, 6H), 1.80-1.72 (m, 2H), 1.58-1.48 (m, 4H), 1.43-1.34 (m, 2H). m/z: [ESI⁺] 476 (M+H)⁺, (C₂₆H₂₉N₅O₂S).

### 2-(4-(Methylcarbamoyl)phenyl)-N-((1-methylpyrrolidin-3-yl)methyl)benzo[d]imidazo[2,1-b]thiazole-7-carboxamide (Compound 176)

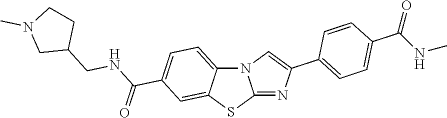

Starting from 2-(4-(methylcarbamoyl)phenyl)benzo[d]imidazo[2,1-b]thiazole-7-carboxylic acid (150 mg, 0.427 mmol); Yield 13 mg (7%), as a white solid. ¹H NMR (400 MHz, DMSO) δ 8.95 (s, 1H), 8.75 (t, J=5.6 Hz, 1H), 8.52 (d, J=1.6 Hz, 1H), 8.46 (q, J=4.4 Hz, 1H), 8.08 (d, J=8.4 Hz, 1H), 8.05 (dd, J=1.6, 8.4 Hz, 1H), 7.95 (d, J=8.6 Hz, 2H), 7.92 (d, J=8.6 Hz, 2H), 3.33-3.26 (m, 2H), 3.18-2.90 (m, 3H), 2.84-2.79 (m, 1H), 2.81 (d, J=4.4 Hz, 3H), 2.62 (s, 3H), 2.64-2.50 (m, 1H), 2.10-2.02 (m, 1H), 1.69-1.67 (m, 1H). m/z: [ESI⁺] 448 (M+H)⁺, (C₂₄H₂₅N₅O₂S).

### N-(3-(diethylamino)propyl)-N-methyl-2-(4-(methylcarbamoyl)phenyl)benzo[d]imidazo[2,1-b]thiazole-7-carboxamide (Compound 180)

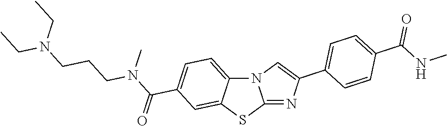 [SMILES: CCN(CC)CCCN(C)C(=O)c1ccc2c(c1)sc1nc(-c3ccc(C(=O)NC)cc3)cn12]

Starting from 2-(4-(methylcarbamoyl)phenyl)benzo[d]imidazo[2,1-b]thiazole-7-carboxylic acid (200 mg, 0.569 mmol); Yield 32 mg (12%), as a white solid. ¹H NMR (400 MHz, DMSO) δ 8.94 (s, 1H), 8.47-8.45 (m, 1H), 8.14 (s, 1H), 8.03 (d, J=8.2 Hz, 1H), 7.95 (d, J=8.6 Hz, 2H), 7.91 (d, J=8.6 Hz, 2H), 7.60 (s, 1H), 3.49 (s, 1H), 3.24 (s, 1H), 2.99 (s, 1.5H), 2.96 (s, 1.5H), 2.81 (d, J=4.4 Hz, 3H), 2.50-2.46 (m, 3H), 2.35-2.20 (m, 3H), 1.75 (s, 1H), 1.66 (s, 1H), 1.00 (s, 3H), 0.75 (s, 3H). m/z: [ESI⁺] 478 (M+H)⁺, (C₂₆H₃₁N₅O₂S).

### N-(3-(diethylamino)propyl)-N-methyl-2-(m-tolyl)benzo[d]imidazo[2,1-b]thiazole-7-carboxamide formate (Compound 181)

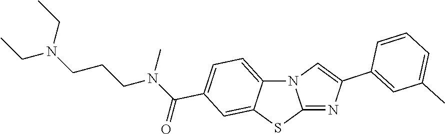 [SMILES: CCN(CC)CCCN(C)C(=O)c1ccc2c(c1)sc1nc(-c3cccc(C)c3)cn12]

Starting from 2-(m-tolyl)benzo[d]imidazo[2,1-b]thiazole-7-carboxylic acid (200 mg, 0.649 mmol); Yield 54 mg (17%), as a white solid. ¹H NMR (400 MHz, DMSO) δ 8.66 (s, 1H), 8.18 (s, 1H), 8.04 (s, 1H), 7.99 (d, J=8.4 Hz, 1H), 7.73 (s, 1H), 7.67 (d, J=8.0 Hz, 1H), 7.55 (dd, J=0.8, 7.2 Hz, 1H), 7.34-7.30 (m, 1H), 7.12 (d, J=7.6 Hz, 1H), 3.42 (t, J=7.2 Hz, 2H), 2.99 (s, 3H), 2.53-2.50 (m, 9H), 1.76-1.69 (m, 2H), 0.94-0.92 (m, 6H). m/z: [ESI⁺] 435 (M+H)⁺, (C₂₅H₃₀N₄OS).

### Azepan-1-yl(2-(p-tolyl)benzo[d]imidazo[2,1-b]thiazol-7-yl)methanone (Compound 102)

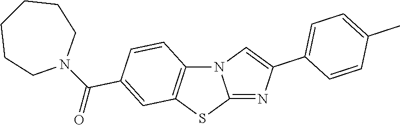 [SMILES: Cc1ccc(-c2cn3c(n2)sc2cc(C(=O)N4CCCCCC4)ccc23)cc1]

Starting from 2-(p-tolyl)benzo[d]imidazo[2,1-b]thiazole-7-carboxylic acid (300 mg, 0.973 mmol); Yield 0.16 g (42%), as a white solid. ¹H NMR (400 MHz, DMSO) δ 8.75 (s, 1H), 8.10 (d, J=1.6 Hz, 1H), 8.00 (d, J=8.4 Hz, 1H), 7.77 (d, J=8.0 Hz, 2H), 7.56 (dd, J=1.6, 8.4 Hz, 1H), 7.26 (d, J=8.0 Hz, 2H), 3.59 (t, J=5.8 Hz, 2H), 3.36 (t, J=11.2 Hz, 2H), 2.34 (s, 3H), 1.75 (s, 2H), 1.65-1.50 (m, 6H). m/z: [ESI⁺] 390 (M+H)⁺, (C₂₃H₂₃N₃OS).

### N-(3-(ethyl(2,2,2-trifluoroethyl)amino)propyl)-2-(m-tolyl)benzo[d]imidazo[2,1-b]thiazole-7-carboxamide (Compound 177)

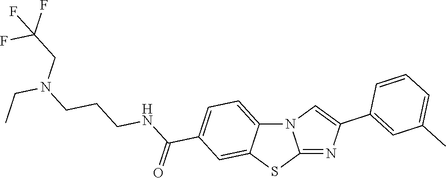 [SMILES: CCN(CCCNC(=O)c1ccc2c(c1)sc1nc(-c3cccc(C)c3)cn12)CC(F)(F)F]

Starting from 2-(m-tolyl)benzo[d]imidazo[2,1-b]thiazole-7-carboxylic acid (75 mg, 0.243 mmol); Yield 8 mg (7%), brown solid. ¹H NMR (400 MHz, DMSO) δ 8.80 (s, 1H), 8.54 (t, J=5.4 Hz, 1H), 8.48 (d, J=1.6 Hz, 1H), 8.04 (d, J=8.4 Hz, 1H), 8.01 (dd, J=1.6, 8.4 Hz, 1H), 7.72 (s, 1H), 7.67 (d, J=7.8 Hz, 1H), 7.34 (dd, J=1.6, 7.6 Hz, 1H), 7.13 (d, J=7.1 Hz, 1H), 3.34-3.26 (m, 2H), 3.22-3.20 (m, 2H), 2.70-2.62 (m, 4H), 2.38 (s, 3H), 1.71-1.69 (m, 2H), 0.99 (t, J=7.2 Hz, 3H). m/z: [ESI⁺]475 (M+H)⁺, (C₂₄H₂₅F₃N₄OS).

### N-(3-(ethyl(2,2,2-trifluoroethyl)amino)propyl)-N-methyl-2-(m-tolyl)benzo[d]imidazo[2,1-b]thiazole-7-carboxamide (Compound 193)

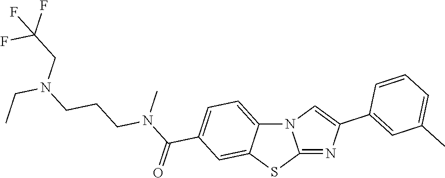 [SMILES: CCN(CCCN(C)C(=O)c1ccc2c(c1)sc1nc(-c3cccc(C)c3)cn12)CC(F)(F)F]

Starting from 2-(m-tolyl)benzo[d]imidazo[2,1-b]thiazole-7-carboxylic acid (200 mg, 0.649 mmol); Yield 93 mg (29%), as a white solid. ¹H NMR (400 MHz, DMSO) δ 8.67 (s, 1H), 8.04 (s, 1H), 8.00 (d, J=8.2 Hz, 1H), 7.73 (s, 1H), 7.68 (d, J=7.8 Hz, 1H), 7.56 (dd, J=1.6, 8.4 Hz, 1H), 7.32 (dd, J=1.6, 7.6 Hz, 1H), 7.13 (d, J=7.4 Hz, 1H), 3.50 (s, 1H), 3.30-3.20 (m, 2H), 3.08 (s, 1H), 2.99 (s, 1.5H), 2.96 (s, 1.5H), 2.50-2.46 (m, 4H), 2.40 (s, 3H), 1.76-1.74 (m, 1H), 1.67-1.65 (m, 1H), 1.03 (t, J=7.2 Hz, 1.5H), 0.80 (t, J=7.2 Hz, 1.5H). m/z: [ESI⁺] 489 (M+H)⁺, (C₂₅H₂₇F₃N₄OS).

### N-(3-(diethylamino)propyl)-2-(2-fluoro-4-(methylcarbamoyl)phenyl)benzo[d]imidazo[2,1-b]thiazole-7-carboxamide (Compound 166)

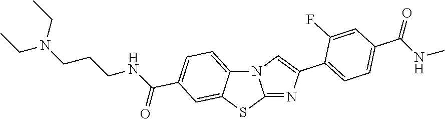 [SMILES: CCN(CC)CCCNC(=O)c1ccc2c(c1)sc1nc(-c3ccc(C(=O)NC)cc3F)cn12]

Starting from 4-(7-((3-(diethylamino)propyl)carbamoyl)benzo[d]imidazo[2,1-b]thiazol-2-yl)-3-fluorobenzoic acid (300 mg, 0.534 mmol); Yield 143 mg (46%), as a white solid. ¹H NMR (400 MHz, DMSO) δ 8.83 (d, J=3.6 Hz, 1H), 8.63 (t, J=5.4 Hz, 1H), 8.58-8.56 (m, 1H), 8.50 (d, J=1.6 Hz, 1H), 8.30 (d, J=8.4 Hz, 1H), 8.24 (dd, J=1.6, 8.0 Hz, 1H), 8.01 (dd, J=1.6, 8.4 Hz, 1H), 7.83-7.74 (m, 2H), 3.33-3.26 (m, 2H), 2.81 (d, J=4.4 Hz, 3H), 2.50-2.46 (m, 6H), 1.68-1.66 (m, 2H), 0.96 (t, J=7.2 Hz, 6H). m/z: [ESI⁺] 482 (M+H)⁺, (C₂₅H₂₈FN₅O₂S).

### N-(3-aminopropyl)-2-(p-tolyl)benzo[d]imidazo[2,1-b]thiazole-7-carboxamide (Compound 115)

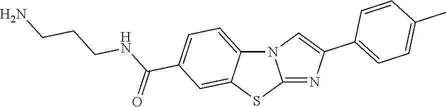 [SMILES: Cc1ccc(-c2cn3c(n2)sc2cc(C(=O)NCCCN)ccc23)cc1]

Starting from 2-(p-tolyl)benzo[d]imidazo[2,1-b]thiazole-7-carboxylic acid (1.00 g, 3.24 mmol); Yield 80 mg (7%), as a white solid. ¹H NMR (400 MHz, DMSO) δ 8.78 (t, J=5.6 Hz, 0.5H), 8.75 (s, 1H), 8.63 (t, J=5.6 Hz, 0.5H), 8.51 (s, 1H), 8.04 (s, 2H), 7.77 (d, J=8.0 Hz, 2H), 7.26 (d, J=8.0 Hz, 2H), 3.37 (t, J=6.0 Hz, 2H), 2.74 (t, J=7.0 Hz, 2H), 2.34 (s, 3H), 1.79-1.61 (m, 2H). m/z: [ESI⁺] 365 (M+H)⁺, (C₂₀H₂₀N₄OS).

### N-(2-aminoethyl)-2-(p-tolyl)benzo[d]imidazo[2,1-b]thiazole-7-carboxamide (Compound 122)

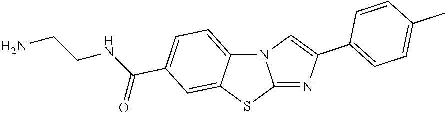 [SMILES: Cc1ccc(-c2cn3c(n2)sc2cc(C(=O)NCCN)ccc23)cc1]

Starting from 2-(p-tolyl)benzo[d]imidazo[2,1-b]thiazole-7-carboxylic acid (1.00 g, 3.24 mmol); Yield 74 mg (7%), as a white solid. ¹H NMR (400 MHz, DMSO) δ 8.76 (s, 1H), 8.57 (t, J=5.6 Hz, 1H), 8.50 (d, J=1.6 Hz, 1H), 8.08-8.04 (m, 2H), 7.77 (d, J=7.8 Hz, 2H), 7.26 (d, J=7.8 Hz, 2H), 3.30 (s, 2H), 2.73 (t, J=6.4 Hz, 2H), 2.34 (s, 3H). m/z: [ESI⁺] 351 (M+H)⁺, (C₁₉H₁₈N₄OS).

### N-propyl-2-(p-tolyl)benzo[d]imidazo[2,1-b]thiazole-7-carboxamide (Compound 108)

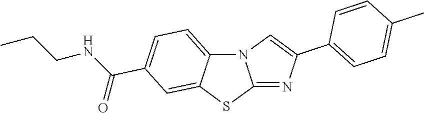 [SMILES: CCCNC(=O)c1ccc2c(c1)sc1nc(-c3ccc(C)cc3)cn12]

Starting from 2-(p-tolyl)benzo[d]imidazo[2,1-b]thiazole-7-carboxylic acid (100 mg, 0.324 mmol); Yield 37 mg (33%), as a white solid. ¹H NMR (400 MHz, DMSO) δ 8.76 (s, 1H), 8.57 (t, J=5.6 Hz, 1H), 8.48 (d, J=1.6 Hz, 1H), 8.04 (s, 2H), 7.77 (d, J=8.0 Hz, 2H), 7.26 (d, J=8.0 Hz, 2H), 3.33-3.26 (m, 2H), 2.34 (s, 3H), 1.62-1.50 (m, 2H), 0.93 (t, J=7.2 Hz, 3H). m/z: [ESI⁺] 350 (M+H)⁺, (C₂₀H₁₉N₃OS).

### N-ethyl-2-(p-tolyl)benzo[d]imidazo[2,1-b]thiazole-7-carboxamide (Compound 109)

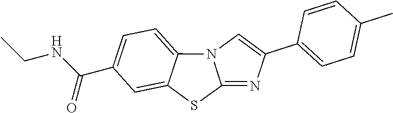 [SMILES: CCNC(=O)c1ccc2c(c1)sc1nc(-c3ccc(C)cc3)cn12]

Starting from 2-(p-tolyl)benzo[d]imidazo[2,1-b]thiazole-7-carboxylic acid (300 mg, 0.973 mmol); Yield 0.24 g (74%), as a white solid. ¹H NMR (400 MHz, DMSO) δ 8.75 (s, 1H), 8.58 (t, J=5.6 Hz, 1H), 8.48 (dd, J=1.6, 2.0 Hz, 1H), 8.03 (s, 2H), 7.76 (d, J=8.0 Hz, 2H), 7.26 (d, J=8.0 Hz, 2H), 3.39-3.28 (m, 2H), 2.34 (s, 3H), 1.16 (t, J=7.2 Hz, 3H). m/z: [ESI⁺] 336 (M+H)⁺, (C₁₉H₁₇N₃OS).

### N-(3-acetamidopropyl)-2-(p-tolyl)benzo[d]imidazo[2,1-b]thiazole-7-carboxamide (Compound 110)

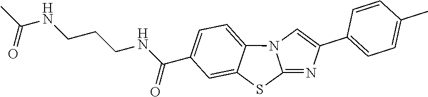 [SMILES: CC(=O)NCCCNC(=O)c1ccc2c(c1)sc1nc(-c3ccc(C)cc3)cn12]

Starting from 2-(p-tolyl)benzo[d]imidazo[2,1-b]thiazole-7-carboxylic acid (100 mg, 0.324 mmol); Yield 74 mg (56%), as a white solid. ¹H NMR (400 MHz, DMSO) δ 8.76 (s, 1H), 8.57 (t, J=5.6 Hz, 1H), 8.48 (d, J=1.6 Hz, 1H), 8.09 (d, J=8.4 Hz, 1H), 8.02 (dd, J=1.6, 8.4 Hz, 1H), 7.88-7.86 (m, 1H), 7.77 (d, J=8.0 Hz, 2H), 7.26 (d, J=8.0 Hz, 2H), 3.32-3.26 (m, 2H), 3.12 (q, J=6.6 Hz, 2H), 2.34 (s, 3H), 1.82 (s, 3H), 1.69-1.67 (m, 2H). m/z: [ESI⁺] 407 (M+H)⁺, (C₂₂H₂₂N₄O₂S).

### N-((3-hydroxyoxetan-3-yl)methyl)-2-(4-(methylcarbamoyl)phenyl)benzo[d]imidazo[2,1-b]thiazole-7-carboxamide (Compound 195)

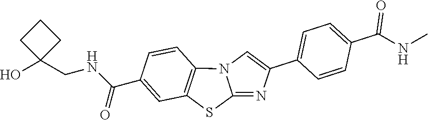 [SMILES: CNC(=O)c1ccc(-c2cn3c(n2)sc2cc(C(=O)NCC4(O)CCC4)ccc23)cc1]

Starting from 2-(4-(methylcarbamoyl)phenyl)benzo[d]imidazo[2,1-b]thiazole-7-carboxylic acid (100 mg, 0.285 mmol); Yield 11 mg (9%), as a white solid. ¹H NMR (400 MHz, DMSO) δ 8.95 (s, 1H), 8.72 (t, J=5.6 Hz, 1H), 8.55 (d, J=1.6 Hz, 1H), 8.46-8.44 (m, 1H), 8.13-8.04 (m, 2H), 7.95 (d, J=8.4 Hz, 2H), 7.91 (d, J=8.4 Hz, 2H), 5.90 (s, 1H), 4.53 (d, J=6.4 Hz, 2H), 4.43 (d, J=6.4 Hz, 2H), 3.62 (d, J=6.0 Hz, 2H), 2.81 (d, J=4.4 Hz, 3H). m/z: [ESI⁺] 437 (M+H)⁺, (C₂₂H₂₀N₄O₄S).

### 4-(7-(4-(2-Amino-2-oxoethyl)piperazine-1-carbonyl)benzo[d]imidazo[2,1-b]thiazol-2-yl)-N-methylbenzamide (Compound 250)

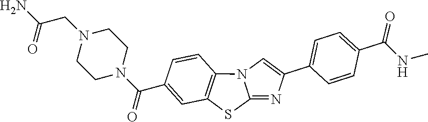 [SMILES: CNC(=O)c1ccc(-c2cn3c(n2)sc2cc(C(=O)N4CCN(CC(N)=O)CC4)ccc23)cc1]

Starting from 2-(4-(methylcarbamoyl)phenyl)benzo[d]imidazo[2,1-b]thiazole-7-carboxylic acid (150 mg, 0.427 mmol); Yield 19 mg (9%), as a white solid. ¹H NMR (400 MHz, DMSO) δ 8.93 (s, 1H), 8.46 (q, J=4.4 Hz, 1H), 8.15 (d, J=1.6 Hz, 1H), 8.02 (d, J=8.4 Hz, 1H), 7.95 (d, J=8.6 Hz, 2H), 7.91 (d, J=8.6 Hz, 2H), 7.61 (dd, J=1.6, 8.4 Hz, 1H), 7.28 (br s, 1H), 7.16 (br s, 1H), 3.68 (s, 2H), 3.46-3.44 (m, 2H), 2.93-2.91 (m, 2H), 2.80 (d, J=4.4 Hz, 3H), 2.50-2.46 (m, 4H). m/z: [ESI⁺] 477 (M+H)⁺, (C₂₄H₂₄N₆O₃S).

### N-((3S,4R)-4-hydroxytetrahydrofuran-3-yl)-2-(4-(methylcarbamoyl)phenyl)benzo[d]imidazo[2,1-b]thiazole-7-carboxamide (Compound 219)

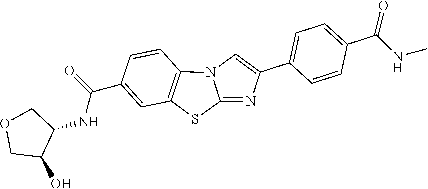 [SMILES: CNC(=O)c1ccc(-c2cn3c(n2)sc2cc(C(=O)N[C@H]4COC[C@@H]4O)ccc23)cc1]

Starting from 2-(4-(methylcarbamoyl)phenyl)benzo[d]imidazo[2,1-b]thiazole-7-carboxylic acid (150 mg, 0.427 mmol); Yield 18 mg (10%), as a white solid. ¹H NMR (400 MHz, DMSO) δ 8.94 (s, 1H), 8.61 (d, J=6.4 Hz, 1H), 8.53 (d, J=1.6 Hz, 1H), 8.45 (q, J=4.4 Hz, 1H), 8.11-8.02 (m, 2H), 7.95 (d, J=8.4 Hz, 2H), 7.91 (d, J=8.4 Hz, 2H), 5.32 (br s, 1H), 4.25 (s, 2H), 4.03 (dd, J=5.4, 9.2 Hz, 1H), 3.95 (dd, J=4.4, 9.4 Hz, 1H), 3.68 (dd, J=3.0, 9.2 Hz, 1H), 3.57 (dd, J=2.0, 9.2 Hz, 1H), 2.81 (d, J=4.4 Hz, 3H). m/z: [ESI⁺] 437 (M+H)⁺, (C₂₂H₂₀N₄O₄S).

### (S)—N-((1,4-dioxan-2-yl)methyl)-2-(4-(methylcarbamoyl)phenyl)benzo[d]imidazo[2,1-b]thiazole-7-carboxamide (Compound 182)

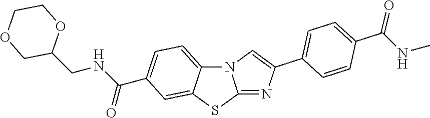 [SMILES: CNC(=O)c1ccc(-c2cn3c(n2)sc2cc(C(=O)NCC4COCCO4)ccc23)cc1]

Starting from 2-(4-(methylcarbamoyl)phenyl)benzo[d]imidazo[2,1-b]thiazole-7-carboxylic acid (120 mg, 0.341 mmol); Yield 30 mg (20%), as a white solid. ¹H NMR (400 MHz, DMSO) δ 8.94 (s, 1H), 8.69 (t, J=5.6 Hz, 1H), 8.52 (s, 1H), 8.46 (q, J=4.4 Hz, 1H), 8.06 (s, 2H), 7.95 (d, J=8.4 Hz, 2H), 7.92 (d, J=8.4 Hz, 2H), 3.77 (dt, J=2.4, 11.6 Hz, 2H), 3.73-3.62 (m, 2H), 3.58 (dt, J=2.4, 11.0 Hz, 1H), 3.48 (dt, J=2.6, 10.8 Hz, 1H), 3.33-3.27 (m, 3H), 2.81 (d, J=4.4 Hz, 3H). m/z: [ESI⁺]451 (M+H)⁺, (C₂₃H₂₂N₄O₄S).

### N-(1-methyl-5-oxopyrrolidin-3-yl)-2-(4-(methylcarbamoyl)phenyl)benzo[d]imidazo[2,1-b]thiazole-7-carboxamide (Compound 236)

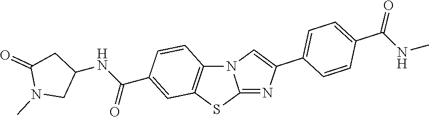 [SMILES: CNC(=O)c1ccc(-c2cn3c(n2)sc2cc(C(=O)NC4CC(=O)N(C)C4)ccc23)cc1]

Starting from 2-(4-(methylcarbamoyl)phenyl)benzo[d]imidazo[2,1-b]thiazole-7-carboxylic acid (150 mg, 0.427 mmol); Yield 48 mg (25%), as a white solid. ¹H NMR (400 MHz, DMSO) δ 8.93 (s, 1H), 8.90 (d, J=6.6 Hz, 1H), 8.53 (d, J=1.6 Hz, 1H), 8.44 (q, J=4.4 Hz, 1H), 8.07 (s, 2H), 7.95 (d, J=8.4 Hz, 2H), 7.92 (d, J=8.4 Hz, 2H), 4.57 (tt, J=3.2, 7.2 Hz, 1H), 3.74 (dd, J=7.2, 10.2 Hz, 1H), 3.31-3.26 (m, 1H), 2.81 (d, J=4.4 Hz, 3H), 2.76 (s, 3H), 2.72-2.64 (m, 1H), 2.42-2.35 (m, 1H). m/z: [ESI⁺] 448 (M+H)⁺, (C₂₃H₂₁N₅O₃S).

### N-(2-(2-oxa-6-azaspiro[3.3]heptan-6-yl)ethyl)-2-(4-(methylcarbamoyl)phenyl)benzo[d]imidazo[2,1-b]thiazole-7-carboxamide formate (Compound 183)

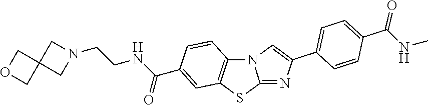 [SMILES: CNC(=O)c1ccc(-c2cn3c(n2)sc2cc(C(=O)NCCN4CC5(COC5)C4)ccc23)cc1]

Starting from 2-(4-(methylcarbamoyl)phenyl)benzo[d]imidazo[2,1-b]thiazole-7-carboxylic acid (150 mg, 0.427 mmol); Yield 44 mg (20%), as a white solid. ¹H NMR (400 MHz, DMSO) δ 8.93 (s, 1H), 8.58 (t, J=5.6 Hz, 1H), 8.50 (s, 1H), 8.47 (q, J=4.4 Hz, 1H), 8.05 (d, J=8.4 Hz, 2H), 8.03 (dd, J=1.6, 8.4 Hz, 2H), 7.95 (d, J=8.4 Hz, 2H), 7.91 (d, J=8.4 Hz, 2H), 4.60 (s, 4H), 3.40 (s, 4H), 3.25 (t, J=6.2 Hz, 2H), 2.81 (d, J=4.4 Hz, 3H), 2.53 (t, J=6.2 Hz, 2H). m/z: [ESI⁺] 476 (M+H)⁺, (C₂₅H₂₅N₅O₃S).

### N-(1-(dimethylamino)-1-oxopropan-2-yl)-2-(4-(methylcarbamoyl)phenyl)benzo[d]imidazo[2,1-b]thiazole-7-carboxamide (Compound 197)

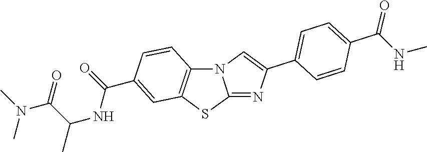

Starting from 2-(4-(methylcarbamoyl)phenyl)benzo[d]imidazo[2,1-b]thiazole-7-carboxylic acid (150 mg, 0.427 mmol); Yield 50 mg (26%), as a white solid. ¹H NMR (400 MHz, DMSO) δ 8.93 (s, 1H), 8.70 (d, J=7.4 Hz, 1H), 8.56 (d, J=1.6 Hz, 1H), 8.45 (q, J=4.4 Hz, 1H), 8.11 (dd, J=1.6, 8.4 Hz, 1H), 8.06 (d, J=8.4 Hz, 1H), 7.95 (d, J=8.4 Hz, 2H), 7.91 (d, J=8.4 Hz, 2H), 5.01-4.90 (m, 1H), 3.09 (s, 3H), 2.87 (s, 3H), 2.81 (d, J=4.4 Hz, 3H), 1.32 (d, J=7.0 Hz, 3H). m/z: [ESI⁺] 450 (M+H)⁺, (C₂₃H₂₃N₅O₃S).

### N-(1-(1H-pyrazol-1-yl)propan-2-yl)-2-(4-(methylcarbamoyl)phenyl)benzo[d]imidazo[2,1-b]thiazole-7-carboxamide (Compound 221)

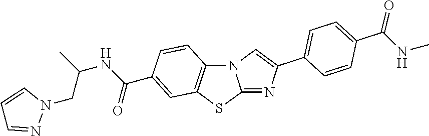 [SMILES: CNC(=O)c1ccc(-c2cn3c(n2)sc2cc(C(=O)NC(C)Cn4cccn4)ccc23)cc1]

Starting from 2-(4-(methylcarbamoyl)phenyl)benzo[d]imidazo[2,1-b]thiazole-7-carboxylic acid (150 mg, 0.427 mmol); Yield 37 mg (19%), as a white solid. ¹H NMR (400 MHz, DMSO) δ 8.93 (s, 1H), 8.49 (d, J=8.2 Hz, 1H), 8.45 (d, J=1.6 Hz, 1H), 8.44 (q, J=4.4 Hz, 1H), 8.06 (d, J=8.4 Hz, 1H), 7.99 (dd, J=1.6, 8.4 Hz, 1H), 7.95 (d, J=8.6 Hz, 2H), 7.92 (d, J=8.6 Hz, 2H), 7.73 (d, J=2.2 Hz, 1H), 7.45 (d, J=1.8 Hz, 1H), 6.23 (t, J=2.0 Hz, 1H), 4.50-4.38 (m, 1H), 4.30 (dd, J=7.0, 13.6 Hz, 1H), 4.23 (dd, J=6.2, 13.6 Hz, 1H), 2.81 (d, J=4.4 Hz, 3H), 1.14 (d, J=6.8 Hz, 3H). m/z: [ESI⁺]459 (M+H)⁺, (C₂₄H₂₂N₆O₂S).

### N-(4-(hydroxymethyl)tetrahydro-2H-pyran-4-yl)-2-(4-(methylcarbamoyl)phenyl)benzo[d]imidazo[2,1-b]thiazole-7-carboxamide (Compound 237)

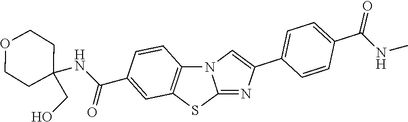 [SMILES: CNC(=O)c1ccc(-c2cn3c(n2)sc2cc(C(=O)NC4(CO)CCOCC4)ccc23)cc1]

Starting from 2-(4-(methylcarbamoyl)phenyl)benzo[d]imidazo[2,1-b]thiazole-7-carboxylic acid (150 mg, 0.427 mmol); Yield 28 mg (14%), as a white solid. ¹H NMR (400 MHz, DMSO) δ 8.94 (s, 1H), 8.52 (d, J=1.6 Hz, 1H), 8.45 (q, J=4.4 Hz, 1H), 8.04 (s, 2H), 7.95 (d, J=8.4 Hz, 2H), 7.91 (d, J=8.4 Hz, 2H), 7.77 (s, 1H), 4.83 (t, J=5.8 Hz, 1H), 3.70 (dt, J=3.8, 11.6 Hz, 2H), 3.67-3.57 (m, 4H), 2.81 (d, J=4.4 Hz, 3H), 2.23 (d, J=13.4 Hz, 2H), 1.70-1.58 (m, 2H). m/z: [ESI⁺] 465 (M+H)⁺, (C₂₄H₂₄N₄O₄S).

### 2-(4-(Methylcarbamoyl)phenyl)-N-(2-(4-methylpiperazin-1-yl)ethyl)benzo[d]imidazo[2,1-b]thiazole-7-carboxamide (Compound 184)

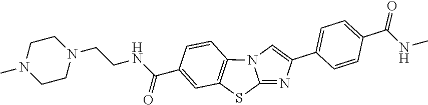

Starting from 2-(4-(methylcarbamoyl)phenyl)benzo[d]imidazo[2,1-b]thiazole-7-carboxylic acid (100 mg, 0.285 mmol); Yield 23 mg (17%), as a white solid. ¹H NMR (400 MHz, DMSO) δ 8.94 (s, 1H), 8.54 (t, J=5.6 Hz, 1H), 8.50 (d, J=1.6 Hz, 1H), 8.46 (q, J=4.4 Hz, 1H), 8.06 (d, J=8.4 Hz, 1H), 8.03 (dd, J=1.6, 8.4 Hz, 1H), 7.95 (d, J=8.4 Hz, 2H), 7.91 (d, J=8.4 Hz, 2H), 3.41 (q, J=6.4 Hz, 2H), 2.81 (d, J=4.4 Hz, 3H), 2.51-2.25 (m, 10H), 2.15 (s, 3H). m/z: [ESI⁺] 477 (M+H)⁺, (C₂₅H₂₈N₆O₂S).

### N-((1-methyl-5-oxopyrrolidin-3-yl)methyl)-2-(4-(methylcarbamoyl)phenyl)benzo[d]imidazo[2,1-b]thiazole-7-carboxamide (Compound 252)

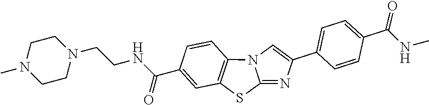

Starting from 2-(4-(methylcarbamoyl)phenyl)benzo[d]imidazo[2,1-b]thiazole-7-carboxylic acid (150 mg, 0.427 mmol); Yield 39 mg (20%), as a white solid. ¹H NMR (400 MHz, DMSO) δ 8.94 (s, 1H), 8.76 (t, J=5.6 Hz, 1H), 8.50 (d, J=1.6 Hz, 1H), 8.46 (q, J=4.4 Hz, 1H), 8.06 (d, J=8.4 Hz, 1H), 8.03 (dd, J=1.6, 8.4 Hz, 1H), 7.95 (d, J=8.4 Hz, 2H), 7.91 (d, J=8.4 Hz, 2H), 3.45 (dd, J=7.8, 9.8 Hz, 1H), 3.16 (dd, J=5.0, 9.8 Hz, 1H), 2.80 (d, J=4.4 Hz, 3H), 2.71 (s, 3H), 2.65-2.52 (m, 2H), 2.43-2.29 (m, 2H), 2.11 (dd, J=6.0, 16.8 Hz, 1H). m/z: [ESI⁺] 462 (M+H)⁺, (C₂₄H₂₃N₅O₃S).

### N-(1-methylazetidin-3-yl)-2-(4-(methylcarbamoyl)phenyl)benzo[d]imidazo[2,1-b]thiazole-7-carboxamide formate (Compound 198)

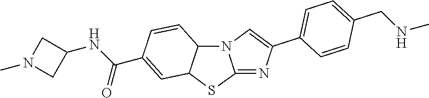

Starting from 2-(4-(methylcarbamoyl)phenyl)benzo[d]imidazo[2,1-b]thiazole-7-carboxylic acid (150 mg, 0.427 mmol); Yield 25 mg (13%), as a white solid. ¹H NMR (400 MHz, DMSO) δ 8.99 (d, J=6.8 Hz, 1H), 8.94 (s, 1H), 8.52 (s, 1H), 8.45 (q, J=4.4 Hz, 1H), 8.21 (s, 1H), 8.07 (s, 2H), 7.95 (d, J=8.4 Hz, 2H), 7.91 (d, J=8.4 Hz, 2H), 4.53 (q, J=7.0 Hz, 1H), 3.72 (t, J=7.6 Hz, 2H), 3.18 (t, J=7.0 Hz, 2H), 2.81 (d, J=4.4 Hz, 3H), 2.37 (s, 3H). m/z: [ESI⁺] 420 (M+H)⁺, (C₂₂H₂₁N₅O₂S).

### N-((1-methyl-5-oxopyrrolidin-2-yl)methyl)-2-(4-(methylcarbamoyl)phenyl)benzo[d]imidazo[2,1-b]thiazole-7-carboxamide (Compound 254)

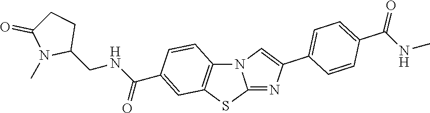 [SMILES: CNC(=O)c1ccc(-c2cn3c(n2)sc2cc(C(=O)NCC4CCC(=O)N4C)ccc23)cc1]

Starting from 2-(4-(methylcarbamoyl)phenyl)benzo[d]imidazo[2,1-b]thiazole-7-carboxylic acid (150 mg, 0.427 mmol); Yield 24 mg (12%), as a white solid. ¹H NMR (400 MHz, DMSO) δ 8.94 (s, 1H), 8.70 (t, J=6.0 Hz, 1H), 8.50 (d, J=1.6 Hz, 1H), 8.45 (q, J=4.4 Hz, 1H), 8.07 (d, J=8.4 Hz, 1H), 8.02 (dd, J=1.6, 8.4 Hz, 1H), 7.95 (d, J=8.4 Hz, 2H), 7.91 (d, J=8.4 Hz, 2H), 3.71 (tt, J=4.0, 8.2 Hz, 1H), 3.60-3.45 (m, 2H), 2.81 (s, 3H), 2.80 (s, 3H), 2.16 (dd, J=4.6, 10.0 Hz, 1H), 2.14-2.09 (m, 1H), 2.09-2.00 (m, 1H), 1.97-1.84 (m, 1H). m/z: [ESI⁺] 462 (M+H)⁺, (C₂₄H₂₃N₅O₃S).

### 2-(4-(Methylcarbamoyl)phenyl)-N-(2-oxo-2-((2,2,2-trifluoroethyl)amino)ethyl)benzo[d]imidazo[2,1-b]thiazole-7-carboxamide (Compound 255)

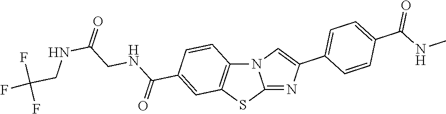 [SMILES: CNC(=O)c1ccc(-c2cn3c(n2)sc2cc(C(=O)NCC(=O)NCC(F)(F)F)ccc23)cc1]

Starting from 2-(4-(methylcarbamoyl)phenyl)benzo[d]imidazo[2,1-b]thiazole-7-carboxylic acid (150 mg, 0.427 mmol). Yield 8 mg (4%), as a white solid. ¹H NMR (400 MHz, DMSO) δ 8.96 (t, J=5.8 Hz, 1H), 8.94 (s, 1H), 8.65 (t, J=6.4 Hz, 1H), 8.56 (s, 1H), 8.46 (q, J=4.4 Hz, 1H), 8.09 (s, 2H), 7.96 (d, J=8.4 Hz, 2H), 7.92 (d, J=8.4 Hz, 2H), 3.99 (d, J=5.4 Hz, 2H), 3.97-3.89 (m, 2H), 2.81 (d, J=3.9 Hz, 3H). m/z: [ESI⁺] 490 (M+H)⁺, (C₂₂H₁₈F₃N₅O₃S).

### N-(2-methoxyethyl)-2-(4-(methylcarbamoyl)phenyl)benzo[d]imidazo[2,1-b]thiazole-7-carboxamide (Compound 201)

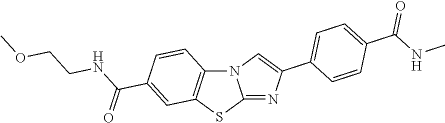

Starting from 2-(4-(methylcarbamoyl)phenyl)benzo[d]imidazo[2,1-b]thiazole-7-carboxylic acid (100 mg, 0.285 mmol). Yield 28 mg (24%), as a white solid. ¹H NMR (400 MHz, DMSO) δ 8.92 (s, 1H), 8.65 (t, J=5.2 Hz, 1H), 8.51 (s, 1H), 8.44 (q, J=4.4 Hz, 1H), 8.06 (s, 2H), 7.95 (d, J=8.4 Hz, 2H), 7.91 (d, J=8.4 Hz, 2H), 3.54-3.43 (m, 4H), 3.29 (s, 3H), 2.81 (d, J=4.4 Hz, 3H). m/z: [ESI⁺] 409 (M+H)⁺, (C₂₁H₂₀N₄O₃S).

### N-(2-(1-methyl-1H-pyrazol-4-yl)ethyl)-2-(4-(methylcarbamoyl)phenyl)benzo[d]imidazo[2,1-b]thiazole-7-carboxamide (Compound 202)

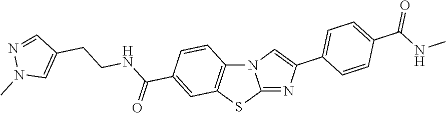

Starting from 2-(4-(methylcarbamoyl)phenyl)benzo[d]imidazo[2,1-b]thiazole-7-carboxylic acid (100 mg, 0.285 mmol). Yield 26 mg (20%), as a white solid. ¹H NMR (400 MHz, DMSO) δ 8.93 (s, 1H), 8.70 (t, J=5.6 Hz, 1H), 8.50 (s, 1H), 8.47 (q, J=4.4 Hz, 1H), 8.08-8.02 (m, 2H), 7.95 (d, J=8.4 Hz, 2H), 7.91 (d, J=8.4 Hz, 2H), 7.55 (s, 1H), 7.31 (s, 1H), 3.78 (s, 3H), 3.44 (q, J=6.8 Hz, 2H), 2.81 (d, J=4.4 Hz, 3H), 2.69 (t, J=7.4 Hz, 2H). m/z: [ESI⁺] 459 (M+H)⁺, (C₂₄H₂₂N₆O₂S).

### N-((4-cyclopropyl-4H-1,2,4-triazol-3-yl)methyl)-2-(4-(methylcarbamoyl)phenyl)benzo[d]imidazo[2,1-b]thiazole-7-carboxamide (Compound 223)

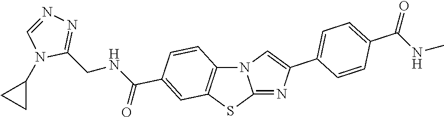 [SMILES: CNC(=O)c1ccc(-c2cn3c(n2)sc2cc(C(=O)NCc4nncn4C4CC4)ccc23)cc1]

Starting from 2-(4-(methylcarbamoyl)phenyl)benzo[d]imidazo[2,1-b]thiazole-7-carboxylic acid (150 mg, 0.427 mmol). Yield 40 mg (20%), as a white solid. ¹H NMR (400 MHz, DMSO) δ 9.17 (t, J=5.4 Hz, 1H), 8.93 (s, 1H), 8.56 (d, J=1.6 Hz, 1H), 8.51 (s, 1H), 8.45 (q, J=4.4 Hz, 1H), 8.10 (dd, J=1.6, 8.4 Hz, 1H), 8.07 (d, J=8.4 Hz, 1H), 7.95 (d, J=8.6 Hz, 2H), 7.91 (d, J=8.6 Hz, 2H), 4.77 (t, J=2.8 Hz, 2H), 3.48-3.38 (m, 1H), 2.81 (d, J=4.4 Hz, 3H), 1.11-0.96 (m, 4H). m/z: [ESI⁺]472 (M+H)⁺, (C₂₄H₂₁N₇O₂S).

### N-(2-(dimethylamino)propyl)-2-(4-(methylcarbamoyl)phenyl)benzo[d]imidazo[2,1-b]thiazole-7-carboxamide (Compound 239)

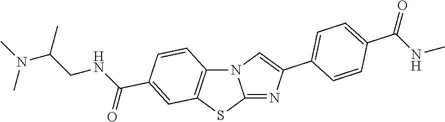

Starting from 2-(4-(methylcarbamoyl)phenyl)benzo[d]imidazo[2,1-b]thiazole-7-carboxylic acid (150 mg, 0.427 mmol). Yield 24 mg (13%), as a white solid. ¹H NMR (400 MHz, DMSO) δ 8.93 (s, 1H), 8.50 (d, J=1.6 Hz, 1H), 8.45 (q, J=4.4 Hz, 1H), 8.41 (t, J=5.6 Hz, 1H), 8.06 (d, J=8.4 Hz, 1H), 8.02 (dd, J=1.6, 8.4 Hz, 1H), 7.95 (d, J=8.6 Hz, 2H), 7.91 (d, J=8.6 Hz, 2H), 3.36-3.30 (m, 1H), 3.27-3.17 (m, 1H), 2.80 (d, J=4.4 Hz, 3H), 2.81-2.76 (m, 1H), 2.22 (s, 6H), 0.94 (d, J=6.4 Hz, 3H). m/z: [ESI⁺] 436 (M+H)⁺, (C₂₃H₂₅N₅O₂S).

### N-(2-methoxycyclopropyl)-2-(4-(methylcarbamoyl)phenyl)benzo[d]imidazo[2,1-b]thiazole-7-carboxamide (Compound 240)

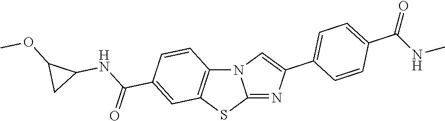

Starting from 2-(4-(methylcarbamoyl)phenyl)benzo[d]imidazo[2,1-b]thiazole-7-carboxylic acid (150 mg, 0.427 mmol). Yield 30 mg (17%), as a white solid. ¹H NMR (400 MHz, DMSO) δ 8.93 (s, 1H), 8.51 (s, 1H), 8.48-8.42 (m, 2H), 8.06 (s, 2H), 7.95 (d, J=8.6 Hz, 2H), 7.91 (d, J=8.6 Hz, 2H), 3.28 (s, 3H), 3.29-3.22 (m, 1H), 2.90 (qd, J=5.2, 8.8 Hz, 1H), 2.81 (d, J=4.4 Hz, 3H), 0.98 (td, J=6.8, 8.8 Hz, 1H), 0.92 (dt, J=4.0, 6.0 Hz, 1H). m/z: [ESI⁺] 421 (M+H)⁺, (C₂₂H₂₀N₄O₃S).

### (S)-2-(4-(methylcarbamoyl)phenyl)-N-(1-methylpyrrolidin-3-yl)benzo[d]imidazo[2,1-b]thiazole-7-carboxamide (Compound 225)

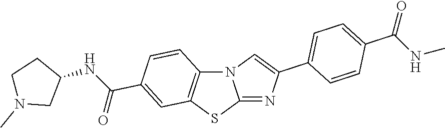

Starting from 2-(4-(methylcarbamoyl)phenyl)benzo[d]imidazo[2,1-b]thiazole-7-carboxylic acid (150 mg, 0.427 mmol). Yield 18 mg (10%), as a white solid. ¹H NMR (400 MHz, DMSO) δ 8.94 (s, 1H), 8.62 (d, J=6.8 Hz, 1H), 8.52 (d, J=1.6 Hz, 1H), 8.45 (q, J=4.4 Hz, 1H), 8.08 (d, J=8.4 Hz, 1H), 8.06 (dd, J=1.6, 8.4 Hz, 1H), 7.95 (d, J=8.6 Hz, 2H), 7.91 (d, J=8.6 Hz, 2H), 4.46-4.44 (m, 1H), 2.86-2.80 (m, 1H), 2.81 (d, J=4.3 Hz, 3H), 2.71 (d, J=7.2 Hz, 1H), 2.59-2.51 (m, 2H), 2.34 (s, 3H), 2.28-2.15 (m, 1H), 1.89-1.77 (m, 1H). m/z: [ESI⁺] 434 (M+H)⁺, (C₂₃H₂₃N₅O₂S).

### N-((3-hydroxycyclobutyl)methyl)-2-(4-(methylcarbamoyl)phenyl)benzo[d]imidazo[2,1-b]thiazole-7-carboxamide (Compound 226)

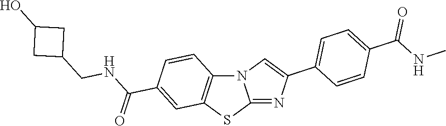 [SMILES: CNC(=O)c1ccc(-c2cn3c(n2)sc2cc(C(=O)NCC4CC(O)C4)ccc23)cc1]

Starting from 2-(4-(methylcarbamoyl)phenyl)benzo[d]imidazo[2,1-b]thiazole-7-carboxylic acid (150 mg, 0.427 mmol). Yield 50 mg (27%), as a white solid. ¹H NMR (400 MHz, DMSO) δ 8.92 (s, 1H), 8.57 (t, J=5.6 Hz, 1H), 8.49 (d, J=1.6 Hz, 1H), 8.44 (q, J=4.4 Hz, 1H), 8.05 (s, 2H), 7.95 (d, J=8.6 Hz, 2H), 7.91 (d, J=8.6 Hz, 2H), 4.95 (d, J=6.4 Hz, 1H), 4.26-4.20 (m, 0.13H), 3.97-3.84 (m, 0.86H), 3.30-3.26 (m, 2H), 2.81 (d, J=4.4 Hz, 3H), 2.32-2.21 (m, 2H), 2.13-2.03 (m, 0.3H), 2.02-1.88 (m, 1H), 1.57 (m, 1.7H). (a mixture of cis/trans with a ratio of 1:6.6). m/z: [ESI⁺] 435 (M+H)⁺, (C₂₃H₂₂N₄O₃S).

### N-(3-(dimethylamino)cyclobutyl)-2-(4-(methylcarbamoyl)phenyl)benzo[d]imidazo[2,1-b]thiazole-7-carboxamide (Compound 269)

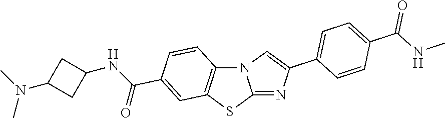

Starting from 2-(4-(methylcarbamoyl)phenyl)benzo[d]imidazo[2,1-b]thiazole-7-carboxylic acid (150 mg, 0.427 mmol). Yield 15 mg (8%), as a white solid. ¹H NMR (400 MHz, DMSO) δ 8.94 (s, 0.4H), 8.93 (s, 0.6H), 8.80 (d, J=6.8 Hz, 0.6H), 8.73 (d, J=7.6 Hz, 0.4H), 8.51 (s, 0.4H), 8.50 (d, J=1.6 Hz, 0.6H), 8.46 (q, J=4.4 Hz, 1H), 8.06 (s, 0.8H), 8.05 (s, 1.2H), 7.95 (d, J=8.6 Hz, 2H), 7.91 (d, J=8.6 Hz, 2H), 4.42-4.28 (m, 0.4H), 4.20-4.08 (m, 0.6H), 2.80 (d, J=4.4 Hz, 3H), 2.45-2.35 (m, 2H), 2.28-2.20 (m, 1H), 2.18-2.12 (m, 1H), 2.10 (s, 3H), 2.07 (s, 3H), 1.95-1.81 (m, 1H). (a mixture of cis/trans with a ratio of 3:2). m/z: [ESI⁺] 448 (M+H)⁺, (C₂₄H₂₅N₅O₂S).

### (S)—N-(1-methoxypropan-2-yl)-2-(4-(methylcarbamoyl)phenyl)benzo[d]imidazo[2,1-b]thiazole-7-carboxamide (Compound 186)

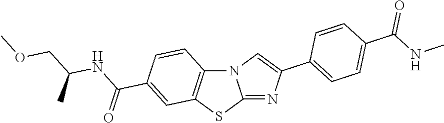

Starting from 2-(4-(methylcarbamoyl)phenyl)benzo[d]imidazo[2,1-b]thiazole-7-carboxylic acid (100 mg, 0.285 mmol). Yield 24 mg (20%), as a white solid. ¹H NMR (400 MHz, DMSO) δ 8.94 (s, 1H), 8.51 (s, 1H), 8.46 (q, J=4.4 Hz, 1H), 8.38 (d, J=8.0 Hz, 1H), 8.06 (s, 2H), 7.95 (d, J=8.6 Hz, 2H), 7.91 (d, J=8.6 Hz, 2H), 4.29-4.18 (m, 1H), 3.44 (dd, J=6.4, 9.6 Hz, 1H), 3.35-3.30 (m, 1H), 3.29 (s, 3H), 2.81 (d, J=4.4 Hz, 3H), 1.17 (d, J=6.8 Hz, 3H). m/z: [ESI⁺] 423 (M+H)⁺, (C₂₂H₂₂N₄O₃S).

### N-(2-methoxypropyl)-2-(4-(methylcarbamoyl)phenyl)benzo[d]imidazo[2,1-b]thiazole-7-carboxamide (Compound 203)

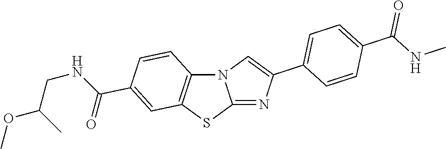

Starting from 2-(4-(methylcarbamoyl)phenyl)benzo[d]imidazo[2,1-b]thiazole-7-carboxylic acid (150 mg, 0.427 mmol). Yield 36 mg (20%), as a white solid. ¹H NMR (400 MHz, DMSO) δ 8.93 (s, 1H), 8.60 (t, J=6.4 Hz, 1H), 8.52 (s, 1H), 8.46 (q, J=4.4 Hz, 1H), 8.06 (s, 2H), 7.95 (d, J=8.6 Hz, 2H), 7.91 (d, J=8.6 Hz, 2H), 3.52-3.50 (m, 1H), 3.38-3.33 (m, 2H), 3.30 (s, 3H), 2.81 (d, J=4.4 Hz, 3H), 1.12 (d, J=6.2 Hz, 3H). m/z: [ESI⁺] 473 (M+H)⁺, (C₂₂H₂₂N₄O₃S).

### (S)—N-(1-(1-methyl-1H-pyrazol-5-yl)propyl)-2-(4-(methylcarbamoyl)phenyl)benzo[d]imidazo[2,1-b]thiazole-7-carboxamide (Compound 242)

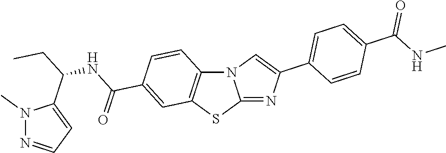

Starting from 2-(4-(methylcarbamoyl)phenyl)benzo[d]imidazo[2,1-b]thiazole-7-carboxylic acid (150 mg, 0.427 mmol). Yield 37 mg (18%), as a white solid. ¹H NMR (400 MHz, DMSO) δ 8.93 (s, 1H), 8.85 (d, J=8.4 Hz, 1H), 8.53 (d, J=1.6 Hz, 1H), 8.44 (q, J=4.4 Hz, 1H), 8.06 (s, 2H), 7.95 (d, J=8.6 Hz, 2H), 7.91 (d, J=8.6 Hz, 2H), 7.34 (d, J=1.6 Hz, 1H), 6.27 (d, J=1.6 Hz, 1H), 5.18 (q, J=7.8 Hz, 1H), 3.83 (s, 3H), 2.81 (d, J=4.4 Hz, 3H), 1.99-1.87 (m, 2H), 0.96 (t, J=7.2 Hz, 3H). m/z: [ESI⁺] 473 (M+H)⁺, (C₂₅H₂₄N₆O₂S).

### (S)—N-(1-methyl-2-oxoazepan-3-yl)-2-(4-(methylcarbamoyl)phenyl)benzo[d]imidazo[2,1-b]thiazole-7-carboxamide (Compound 228)

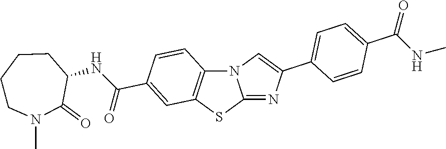 [SMILES: CNC(=O)c1ccc(-c2cn3c(n2)sc2cc(C(=O)N[C@H]4CCCCN(C)C4=O)ccc23)cc1]

Starting from 2-(4-(methylcarbamoyl)phenyl)benzo[d]imidazo[2,1-b]thiazole-7-carboxylic acid (150 mg, 0.427 mmol). Yield 33 mg (16%), as a white solid. ¹H NMR (400 MHz, DMSO) δ 8.94 (s, 1H), 8.56 (d, J=1.6 Hz, 1H), 8.45 (d, J=4.4 Hz, 1H), 8.07 (s, 2H), 7.95 (d, J=8.6 Hz, 2H), 7.91 (d, J=8.6 Hz, 2H), 4.82 (dd, J=1.8, 11.2 Hz, 1H), 3.70 (dd, J=11.2, 15.2 Hz, 1H), 3.33-3.26 (m, 1H), 2.95 (s, 3H), 2.81 (d, J=4.4 Hz, 3H), 1.97-1.85 (m, 2H), 1.80-1.72 (m, 2H), 1.63-1.49 (m, 1H), 1.47-1.37 (m, 1H). m/z: [ESI⁺] 476 (M+H)⁺, (C₂₅H₂₅N₅O₃S).

### N-(2-(2-ethyl-1H-imidazol-1-yl)ethyl)-2-(4-(methylcarbamoyl)phenyl)benzo[d]imidazo[2,1-b]thiazole-7-carboxamide hemiformate (Compound 243)

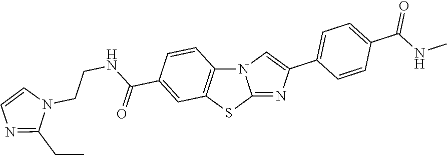 [SMILES: CCc1nccn1CCNC(=O)c1ccc2c(c1)sc1nc(-c3ccc(C(=O)NC)cc3)cn12]

Starting from 2-(4-(methylcarbamoyl)phenyl)benzo[d]imidazo[2,1-b]thiazole-7-carboxylic acid (150 mg, 0.427 mmol). Yield 42 mg (20%), off-as a white solid. ¹H NMR (400 MHz, DMSO) δ 8.92 (d, J=1.4 Hz, 1H), 8.78 (t, J=5.6 Hz, 1H), 8.47 (d, J=1.6 Hz, 1H), 8.45 (d, J=4.4 Hz, 1H), 8.06 (d, J=8.4 Hz, 1H), 8.00 (dd, J=1.6, 8.4 Hz, 1H), 7.95 (d, J=8.6 Hz, 2H), 7.91 (d, J=8.6 Hz, 2H), 7.08 (d, J=1.2 Hz, 1H), 6.78 (d, J=1.2 Hz, 1H), 4.11 (t, J=6.4 Hz, 2H), 3.62-3.55 (m, 2H), 2.81 (d, J=4.4 Hz, 3H), 2.71-2.60 (m, 2H), 1.19 (t, J=7.6 Hz, 3H). m/z: [ESI⁺] 473 (M+H)⁺, (C₂₅H₂₄N₆O₂S).

### N-(4-hydroxybutan-2-yl)-2-(4-(methylcarbamoyl)phenyl)benzo[d]imidazo[2,1-b]thiazole-7-carboxamide (Compound 187)

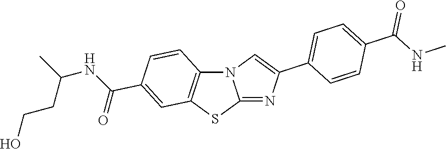 [SMILES: CNC(=O)c1ccc(-c2cn3c(n2)sc2cc(C(=O)NC(C)CCO)ccc23)cc1]

Starting from 2-(4-(methylcarbamoyl)phenyl)benzo[d]imidazo[2,1-b]thiazole-7-carboxylic acid (100 mg, 0.285 mmol). Yield 23 mg (19%), as an off-white solid. ¹H NMR (400 MHz, DMSO) δ 8.94 (s, 1H), 8.50 (d, J=1.6 Hz, 1H), 8.46 (q, J=4.4 Hz, 1H), 8.34 (d, J=8.0 Hz, 1H), 8.05 (s, 2H), 7.95 (d, J=8.6 Hz, 2H), 7.91 (d, J=8.6 Hz, 2H), 4.46 (t, J=5.2 Hz, 1H), 4.21-4.12 (m, 1H), 3.52-3.43 (m, 2H), 2.81 (d, J=4.4 Hz, 3H), 1.81-1.60 (m, 2H), 1.19 (d, J=6.4 Hz, 3H). m/z: [ESI⁺] 423 (M+H)⁺, (C₂₂H₂₂N₄O₃S).

### (S)—N-(1-hydroxybutan-2-yl)-2-(4-(methylcarbamoyl)phenyl)benzo[d]imidazo[2,1-b]thiazole-7-carboxamide (Compound 188)

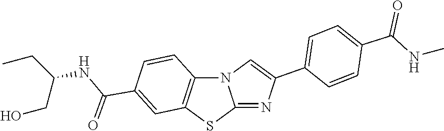 [SMILES: CC[C@@H](CO)NC(=O)c1ccc2c(c1)sc1nc(-c3ccc(C(=O)NC)cc3)cn12]

Starting from 2-(4-(methylcarbamoyl)phenyl)benzo[d]imidazo[2,1-b]thiazole-7-carboxylic acid (100 mg, 0.285 mmol). Yield 41 mg (34%), as an off-white solid. ¹H NMR (400 MHz, DMSO) δ 8.95 (s, 1H), 8.53 (d, J=1.6 Hz, 1H), 8.46 (q, J=4.4 Hz, 1H), 8.16 (d, J=8.4 Hz, 1H), 8.12-8.02 (m, 2H), 7.95 (d, J=8.6 Hz, 2H), 7.91 (d, J=8.6 Hz, 2H), 4.71 (t, J=5.8 Hz, 1H), 3.95-3.84 (m, 1H), 3.55-3.37 (m, 2H), 2.81 (d, J=4.4 Hz, 3H), 1.75-1.64 (m, 1H), 1.55-1.42 (m, 1H), 0.91 (t, J=7.4 Hz, 3H). m/z: [ESI⁺] 423 (M+H)⁺, (C₂₂H₂₂N₄O₃S).

### 2-(4-(Methylcarbamoyl)phenyl)-N-((tetrahydrofuran-2-yl)methyl)benzo[d]imidazo[2,1-b]thiazole-7-carboxamide (Compound 204)

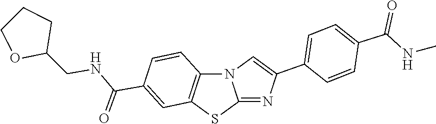 [SMILES: CNC(=O)c1ccc(-c2cn3c(n2)sc2cc(C(=O)NCC4CCCO4)ccc23)cc1]

Starting from 2-(4-(methylcarbamoyl)phenyl)benzo[d]imidazo[2,1-b]thiazole-7-carboxylic acid (100 mg, 0.285 mmol). Yield 51 mg (41%), as an off-white solid. ¹H NMR (400 MHz, DMSO) δ 8.93 (s, 1H), 8.67 (t, J=5.8 Hz, 1H), 8.52 (d, J=1.6 Hz, 1H), 8.46 (q, J=4.4 Hz, 1H), 8.06 (s, 2H), 7.95 (d, J=8.6 Hz, 2H), 7.91 (d, J=8.6 Hz, 2H), 4.02-4.00 (m, 1H), 3.80 (dt, J=6.4, 8.0 Hz, 1H), 3.65 (q, J=7.2 Hz, 1H), 3.37-3.26 (m, 2H), 2.81 (d, J=4.4 Hz, 3H), 1.98-1.89 (m, 1H), 1.88-1.80 (m, 2H), 1.68-1.55 (m, 1H). m/z: [ESI⁺] 435 (M+H)⁺, (C₂₃H₂₂N₄O₃S).

### N-(2-aminocyclohexyl)-2-(4-(methylcarbamoyl)phenyl)benzo[d]imidazo[2,1-b]thiazole-7-carboxamide (Compound 205)

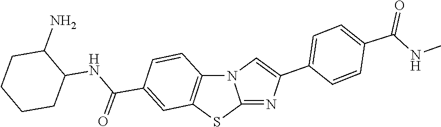 [SMILES: CNC(=O)c1ccc(-c2cn3c(n2)sc2cc(C(=O)NC4CCCCC4N)ccc23)cc1]

Starting from 2-(4-(methylcarbamoyl)phenyl)benzo[d]imidazo[2,1-b]thiazole-7-carboxylic acid (100 mg, 0.285 mmol). Yield 10 mg (8%), as an off-white solid. ¹H NMR (400 MHz, DMSO) δ 8.95 (s, 1H), 8.55 (d, J=1.6 Hz, 1H), 8.47 (q, J=4.4 Hz, 1H), 8.40 (d, J=6.2 Hz, 1H), 8.12-8.05 (m, 2H), 7.95 (d, J=8.6 Hz, 2H), 7.91 (d, J=8.6 Hz, 2H), 4.03 (s, 0.3H), 3.67 (d, J=9.2 Hz, 0.7H), 2.81 (d, J=4.4 Hz, 3H), 2.75-2.68 (m, 1H), 1.95-1.86 (m, 1H), 1.73-1.46 (m, 3H), 1.40-1.20 (m, 4H). (a mixture of cis/trans with a ratio of 3:7). m/z: [ESI⁺] 448 (M+H)⁺, (C₂₄H₂₅N₅O₂S).

### (N-(3-(2-(hydroxymethyl)pyrrolidin-1-yl)propyl)-2-(4-(methylcarbamoyl)phenyl)benzo[d]imidazo[2,1-b]thiazole-7-carboxamideformate (Compound 271)

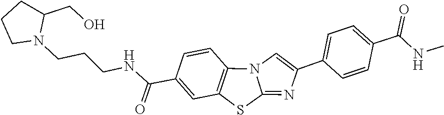 [SMILES: CNC(=O)c1ccc(-c2cn3c(n2)sc2cc(C(=O)NCCCN4CCCC4CO)ccc23)cc1]

Starting from 2-(4-(methylcarbamoyl)phenyl)benzo[d]imidazo[2,1-b]thiazole-7-carboxylic acid (150 mg, 0.427 mmol). Yield 27 mg (12%), as a white solid. ¹H NMR (400 MHz, DMSO) δ 8.94 (d, J=1.6 Hz, 1H), 8.70 (t, J=6.8 Hz, 1H), 8.50 (d, J=1.6 Hz, 1H), 8.46 (q, J=4.4 Hz, 1H), 8.21 (s, 1H), 8.06 (d, J=8.4 Hz, 1H), 8.03 (dd, J=1.6, 8.4 Hz, 1H), 7.95 (d, J=8.6 Hz, 2H), 7.91 (d, J=8.6 Hz, 2H), 3.47-3.35 (m, 4H), 3.17-3.09 (m, 1H), 3.00-2.90 (m, 1H), 2.81 (d, J=4.4 Hz, 3H), 2.65-2.53 (m, 1H), 2.49-2.36 (m, 1H), 2.31-2.17 (m, 1H), 1.90-1.80 (m, 1H), 1.79-1.66 (m, 3H), 1.66-1.52 (m, 2H). m/z: [ESI⁺] 492 (M+H)⁺, (C₂₆H₂₉N₅O₃S).

### N-(3-methoxypropyl)-2-(4-(methylcarbamoyl)phenyl)benzo[d]imidazo[2,1-b]thiazole-7-carboxamide (Compound 189)

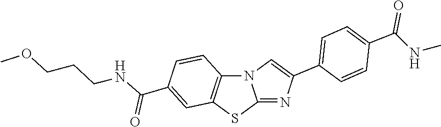

Starting from 2-(4-(methylcarbamoyl)phenyl)benzo[d]imidazo[2,1-b]thiazole-7-carboxylic acid (150 mg, 0.427 mmol). Yield 32 mg (18%), as a white solid. ¹H NMR (400 MHz, DMSO) δ 8.93 (s, 1H), 8.61 (t, J=5.6 Hz, 1H), 8.50 (s, 1H), 8.46 (q, J=4.4 Hz, 1H), 8.08-8.03 (m, 2H), 7.95 (d, J=8.6 Hz, 2H), 7.91 (d, J=8.6 Hz, 2H), 3.40 (t, J=6.4 Hz, 2H), 3.35-3.26 (m, 2H), 3.26 (s, 3H), 2.81 (d, J=4.4 Hz, 3H), 1.79 (p, J=6.6 Hz, 2H). m/z: [ESI⁺] 423 (M+H)⁺, (C₂₂H₂₂N₄O₃S).

### N-(2-methyl-2-morpholinopropyl)-2-(4-(methylcarbamoyl)phenyl)benzo[d]imidazo[2,1-b]thiazole-7-carboxamide (Compound 244)

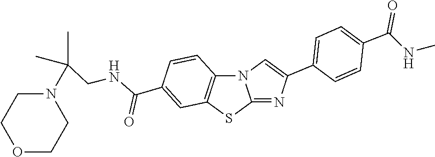 [SMILES: CNC(=O)c1ccc(-c2cn3c(n2)sc2cc(C(=O)NCC(C)(C)N4CCOCC4)ccc23)cc1]

Starting from 2-(4-(methylcarbamoyl)phenyl)benzo[d]imidazo[2,1-b]thiazole-7-carboxylic acid (150 mg, 0.427 mmol). Yield 72 mg (34%), as an off-white solid. ¹H NMR (400 MHz, DMSO) δ 8.94 (s, 1H), 8.51 (d, J=1.6 Hz, 1H), 8.45 (q, J=4.4 Hz, 1H), 8.06 (d, J=8.4 Hz, 1H), 8.03 (dd, J=1.6, 8.4 Hz, 1H), 7.95 (d, J=8.6 Hz, 2H), 7.91 (d, J=8.6 Hz, 2H), 3.59 (s, 4H), 3.33-3.26 (m, 2H), 2.81 (d, J=4.4 Hz, 3H), 2.54 (s, 4H), 1.05 (s, 6H). m/z: [ESI⁺] 492 (M+H)⁺, (C₂₆H₂₉N₅O₃S).

### N-((1s,3s)-3-methoxycyclobutyl)-2-(4-(methylcarbamoyl)phenyl)benzo[d]imidazo[2,1-b]thiazole-7-carboxamide (Compound 245)

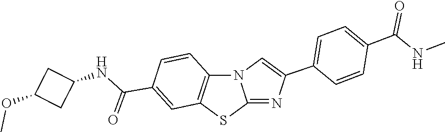

Starting from 2-(4-(methylcarbamoyl)phenyl)benzo[d]imidazo[2,1-b]thiazole-7-carboxylic acid (150 mg, 0.427 mmol). Yield 32 mg (17%), as an off-white solid. ¹H NMR (400 MHz, DMSO) δ 8.93 (s, 1H), 8.74 (d, J=7.6 Hz, 1H), 8.50 (s, 1H), 8.44 (q, J=4.4 Hz, 1H), 8.06 (s, 2H), 7.95 (d, J=8.6 Hz, 2H), 7.91 (d, J=8.6 Hz, 2H), 4.07 (qt, J=7.6, 9.2 Hz, 1H), 3.70-3.59 (m, 1H), 3.17 (s, 3H), 2.81 (d, J=4.4 Hz, 3H), 2.72-2.55 (m, 2H), 2.04-1.92 (m, 2H). m/z: [ESI⁺] 435 (M+H)⁺, (C₂₃H₂₂N₄O₃S).

### 2-(4-(Methylcarbamoyl)phenyl)-N-(3-(trifluoromethyl)oxetan-3-yl)benzo[d]imidazo[2,1-b]thiazole-7-carboxamide (Compound 206)

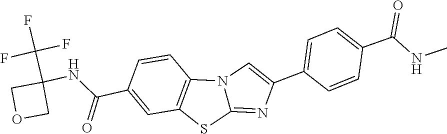 [SMILES: CNC(=O)c1ccc(-c2cn3c(n2)sc2cc(C(=O)NC4(C(F)(F)F)COC4)ccc23)cc1]

Starting from 2-(4-(methylcarbamoyl)phenyl)benzo[d]imidazo[2,1-b]thiazole-7-carboxylic acid (150 mg, 0.427 mmol). Yield 20 mg (10%), as an off-white solid. ¹H NMR (400 MHz, DMSO) δ 9.56 (s, 1H), 8.95 (s, 1H), 8.55 (d, J=1.6 Hz, 1H), 8.45 (q, J=4.4 Hz, 1H), 8.06 (d, J=8.4 Hz, 1H), 8.03 (dd, J=1.6, 8.4 Hz, 1H), 7.95 (d, J=8.6 Hz, 2H), 7.91 (d, J=8.6 Hz, 2H), 4.94 (d, J=8.0 Hz, 2H), 4.81 (d, J=8.0 Hz, 2H), 2.81 (d, J=4.4 Hz, 3H). m/z: [ESI⁺] 475 (M+H)⁺, (C₂₂H₁₇F₃N₄03S).

### (S)-2-(4-(methylcarbamoyl)phenyl)-N-(1-methylpiperidin-3-yl)benzo[d]imidazo[2,1-b]thiazole-7-carboxamide (Compound 207)

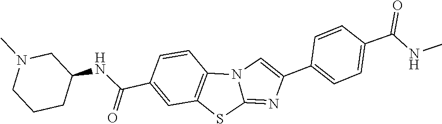

Starting from 2-(4-(methylcarbamoyl)phenyl)benzo[d]imidazo[2,1-b]thiazole-7-carboxylic acid (150 mg, 0.427 mmol). Yield 53 mg (28%), as a white solid. ¹H NMR (400 MHz, DMSO) δ 8.93 (s, 1H), 8.50 (d, J=1.6 Hz, 1H), 8.44 (q, J=4.4 Hz, 1H), 8.05 (s, 2H), 7.95 (d, J=8.6 Hz, 2H), 7.91 (d, J=8.6 Hz, 2H), 3.96 (tt, J=4.0, 9.8 Hz, 1H), 2.86-2.81 (m, 1H), 2.81 (d, J=4.4 Hz, 3H), 2.69-2.60 (m, 1H), 2.19 (s, 3H), 1.88 (t, J=10.1 Hz, 2H), 1.93-1.77 (m, 3H), 1.71 (td, J=3.6, 12.8 Hz, 1H), 1.56 (dd, J=10.0, 13.8 Hz, 1H), 1.34 (dq J=4.0, 11.9 Hz, 1H). m/z: [ESI⁺] 448 (M+H)⁺, (C₂₄H₂₅N₅O₂S).

### N-(2-(dimethylamino)butyl)-2-(4-(methylcarbamoyl)phenyl)benzo[d]imidazo[2,1-b]thiazole-7-carboxamide (Compound 208)

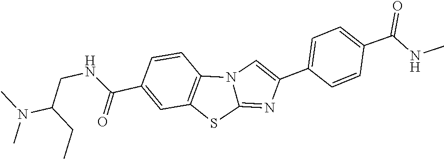

Starting from 2-(4-(methylcarbamoyl)phenyl)benzo[d]imidazo[2,1-b]thiazole-7-carboxylic acid (100 mg, 0.285 mmol). Yield 12 mg (9%), as a white solid. ¹H NMR (400 MHz, DMSO) δ 8.94 (s, 1H), 8.50 (d, J=1.6 Hz, 1H), 8.46 (q, J=4.4 Hz, 1H), 8.39 (t, J=5.6 Hz, 1H), 8.06 (d, J=8.4 Hz, 1H), 8.03 (dd, J=1.6, 8.4 Hz, 1H), 7.96 (d, J=8.6 Hz, 2H), 7.92 (d, J=8.6 Hz, 2H), 3.44-3.34 (m, 1H), 3.33-3.20 (m, 1H), 2.81 (d, J=4.4 Hz, 3H), 2.60-2.51 (m, 1H), 2.27 (s, 6H), 1.47 (dq, J=7.2, 14.6 Hz, 1H), 1.36 (qd, J=7.2, 14.2 Hz, 1H), 0.96 (q, J=7.4 Hz, 3H). m/z: [ESI⁺] 450 (M+H)⁺, (C₂₄H₂₇N₅O₂S).

### (S)-2-(4-(methylcarbamoyl)phenyl)-N-(1-(tetrahydro-2H-pyran-4-yl)pyrrolidin-3-yl)benzo[d]imidazo[2,1-b]thiazole-7-carboxamide formate (Compound 209)

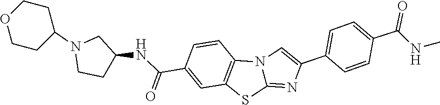 [SMILES: CNC(=O)c1ccc(-c2cn3c(n2)sc2cc(C(=O)N[C@H]4CCN(C5CCOCC5)C4)ccc23)cc1]

Starting from 2-(4-(methylcarbamoyl)phenyl)benzo[d]imidazo[2,1-b]thiazole-7-carboxylic acid (140 mg, 0.398 mmol). Yield 60 mg (30%), as an off-white solid. ¹H NMR (400 MHz, DMSO) δ 8.93 (s, 1H), 8.66 (d, J=7.0 Hz, 1H), 8.52 (s, 1H), 8.47-8.45 (m, 1H), 8.19 (s, 1H), 8.06 (d, J=8.4 Hz, 1H), 8.03 (dd, J=1.6, 8.4 Hz, 1H), 7.95 (d, J=8.6 Hz, 2H), 7.91 (d, J=8.6 Hz, 2H), 4.56-4.37 (m, 1H), 3.91-3.79 (m, 2H), 3.30 (dt, J=2.2, 11.8 Hz, 2H), 2.94 (dd, J=7.6, 9.6 Hz, 1H), 2.81 (d, J=4.4 Hz, 3H), 2.83-2.76 (m, 1H), 2.62 (d, J=9.2 Hz, 2H), 2.50-2.46 (m, 1H), 2.23-2.13 (m, 1H), 1.87-1.75 (m, 3H), 1.49-1.33 (m, 2H). m/z: [ESI⁺] 504 (M+H)⁺, (C₂₇H₂₉N₅O₃S).

### 2-(4-(Methylcarbamoyl)phenyl)-N-((3-methyltetrahydrofuran-3-yl)methyl)benzo[d]imidazo[2,1-b]thiazole-7-carboxamide (Compound 210)

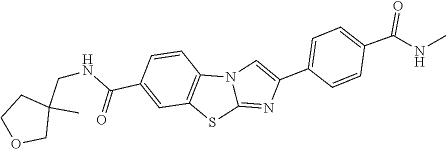 [SMILES: CNC(=O)c1ccc(-c2cn3c(n2)sc2cc(C(=O)NCC4(C)CCOC4)ccc23)cc1]

Starting from 2-(4-(methylcarbamoyl)phenyl)benzo[d]imidazo[2,1-b]thiazole-7-carboxylic acid (100 mg, 0.285 mmol). Yield 34 mg (27%), as an off-white solid. ¹H NMR (400 MHz, DMSO) δ 8.95 (s, 1H), 8.61 (t, J=6.4 Hz, 1H), 8.53 (d, J=1.2 Hz, 1H), 8.47-8.45 (m, 1H), 8.06 (s, 2H), 7.95 (d, J=8.6 Hz, 2H), 7.91 (d, J=8.6 Hz, 2H), 3.86-3.70 (m, 2H), 3.66 (d, J=8.4 Hz, 1H), 3.40-3.25 (m, 3H), 2.81 (d, J=4.4 Hz, 3H), 1.92-1.90 (m, 1H), 1.59-1.57 (m, 1H), 1.09 (s, 3H). m/z: [ESI⁺] 449 (M+H)⁺, (C₂₄H₂₄N₄O₃S).

### (R)-2-(4-(methylcarbamoyl)phenyl)-N-(quinuclidin-3-yl)benzo[d]imidazo[2,1-b]thiazole-7-carboxamide formate (Compound 231)

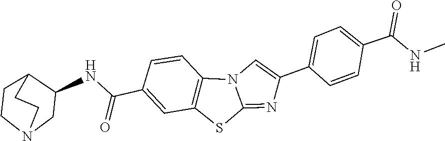 [SMILES: CNC(=O)c1ccc(-c2cn3c(n2)sc2cc(C(=O)N[C@H]4CN5CCC4CC5)ccc23)cc1]

Starting from 2-(4-(methylcarbamoyl)phenyl)benzo[d]imidazo[2,1-b]thiazole-7-carboxylic acid (150 mg, 0.427 mmol). Yield 19 mg (9%), as a white solid. ¹H NMR (400 MHz, DMSO) δ 8.95 (s, 1H), 8.54 (d, J=1.2 Hz, 1H), 8.48 (d, J=6.6 Hz, 1H), 8.46-8.44 (m, 1H), 8.24 (s, 1H), 8.07 (s, 2H), 7.95 (d, J=8.6 Hz, 2H), 7.91 (d, J=8.6 Hz, 2H), 4.07 (s, 1H), 3.32-3.22 (m, 1H), 3.07-2.95 (m, 1H), 2.88-2.75 (m, 4H), 2.81 (d, J=4.4 Hz, 3H), 2.00-1.87 (m, 2H), 1.73-1.65 (m, 2H), 1.49-1.37 (m, 1H). m/z: [ESI⁺] 460 (M+H)⁺, (C₂₅H₂₅N₅O₂S).

### N-(3-(3,5-dimethyl-1H-pyrazol-1-yl)propyl)-2-(4-(methylcarbamoyl)phenyl)benzo[d]imidazo[2,1-b]thiazole-7-carboxamide (Compound 232)

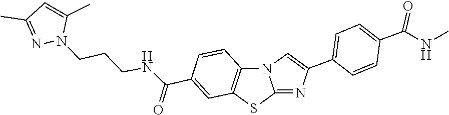

Starting from 2-(4-(methylcarbamoyl)phenyl)benzo[d]imidazo[2,1-b]thiazole-7-carboxylic acid (150 mg, 0.427 mmol). Yield 42 mg (20%), as a white solid. ¹H NMR (400 MHz, DMSO) δ 8.93 (s, 1H), 8.62 (t, J=5.6 Hz, 1H), 8.50 (d, J=1.6 Hz, 1H), 8.46-8.44 (m, 1H), 8.07 (d, J=8.4 Hz, 1H), 8.03 (dd, J=1.6, 8.4 Hz, 1H), 7.95 (d, J=8.6 Hz, 2H), 7.91 (d, J=8.6 Hz, 2H), 5.78 (s, 1H), 4.00 (t, J=7.0 Hz, 2H), 3.33-3.26 (m, 2H), 2.81 (d, J=4.4 Hz, 3H), 2.20 (s, 3H), 2.09 (s, 3H), 2.00 (p, J=7.0 Hz, 2H). m/z: [ESI⁺] 487 (M+H)⁺, (C₂₆H₂₆N₆O₂S).

### N-(2-isopropoxyethyl)-2-(4-(methylcarbamoyl)phenyl)benzo[d]imidazo[2,1-b]thiazole-7-carboxamide (Compound 211)

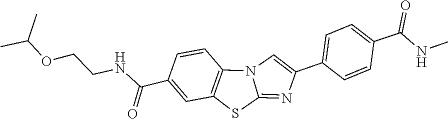 [SMILES: CNC(=O)c1ccc(-c2cn3c(n2)sc2cc(C(=O)NCCOC(C)C)ccc23)cc1]

Starting from 2-(4-(methylcarbamoyl)phenyl)benzo[d]imidazo[2,1-b]thiazole-7-carboxylic acid (100 mg, 0.285 mmol). Yield 16 mg (13%), as a white solid. ¹H NMR (400 MHz, DMSO) δ 8.94 (s, 1H), 8.65 (t, J=5.6 Hz, 1H), 8.52 (s, 1H), 8.47-8.45 (m, 1H), 8.06 (s, 2H), 7.95 (d, J=8.6 Hz, 2H), 7.91 (d, J=8.6 Hz, 2H), 3.61-3.59 (m, 1H), 3.52 (t, J=6.0 Hz, 2H), 3.44 (t, J=5.8 Hz, 2H), 2.81 (d, J=4.4 Hz, 3H), 1.11 (d, J=6.0 Hz, 6H). m/z: [ESI⁺] 437 (M+H)⁺, (C₂₃H₂₄N₄O₃S).

### (R)-2-(4-(methylcarbamoyl)phenyl)-N-((1-methylpiperidin-3-yl)methyl)benzo[d]imidazo[2,1-b]thiazole-7-carboxamide (Compound 212)

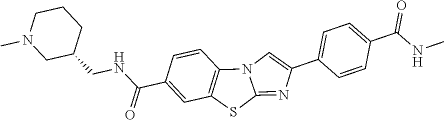

Starting from 2-(4-(methylcarbamoyl)phenyl)benzo[d]imidazo[2,1-b]thiazole-7-carboxylic acid (150 mg, 0.427 mmol). Yield 41 mg (21%), as a white solid. ¹H NMR (400 MHz, DMSO) δ 8.93 (s, 1H), 8.61 (t, J=5.6 Hz, 1H), 8.51 (s, 1H), 8.45-8.43 (m, 1H), 8.24 (s, 1H), 8.05 (s, 2H), 7.95 (d, J=8.6 Hz, 2H), 7.91 (d, J=8.6 Hz, 2H), 3.20 (dq, J=6.8, 13.2 Hz, 2H), 2.81 (d, J=4.4 Hz, 3H), 2.84-2.78 (m, 1H), 2.50-2.46 (m, 1H), 2.22 (s, 3H), 2.01-1.82 (m, 2H), 1.81-1.61 (m, 3H), 1.54-1.40 (m, 1H), 1.04-0.91 (m, 1H). m/z: [ESI⁺] 462 (M+H)⁺, (C₂₅H₂₇N₅O₂S).

### N-((1-ethylpyrrolidin-3-yl)methyl)-2-(4-(methylcarbamoyl)phenyl)benzo[d]imidazo[2,1-b]thiazole-7-carboxamide (Compound 233)

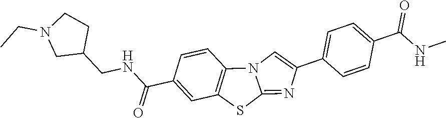 [SMILES: CCN1CCC(CNC(=O)c2ccc3c(c2)sc2nc(-c4ccc(C(=O)NC)cc4)cn23)C1]

Starting from 2-(4-(methylcarbamoyl)phenyl)benzo[d]imidazo[2,1-b]thiazole-7-carboxylic acid (150 mg, 0.427 mmol). Yield 22 mg (11%), as a white solid. ¹H NMR (400 MHz, DMSO) δ 8.93 (s, 1H), 8.65 (t, J=5.6 Hz, 1H), 8.50 (d, J=1.6 Hz, 1H), 8.46-8.44 (m, 1H), 8.07 (d, J=8.4 Hz, 1H), 8.03 (dd, J=1.6, 8.4 Hz, 1H), 7.95 (d, J=8.6 Hz, 2H), 7.91 (d, J=8.6 Hz, 2H), 3.30-3.19 (m, 2H), 2.81 (d, J=4.4 Hz, 3H), 2.65-2.45 (m, 5H), 2.39 (t, J=7.2 Hz, 2H), 1.95-1.82 (m, 1H), 1.53-1.41 (m, 1H), 1.03 (t, J=7.2 Hz, 3H). m/z: [ESI⁺] 462 (M+H)⁺, (C₂₅H₂₇N₅O₂S).

### (R)—N-(2-hydroxy-1-phenylethyl)-2-(4-(methylcarbamoyl)phenyl)benzo[d]imidazo[2,1-b]thiazole-7-carboxamide (Compound 213)

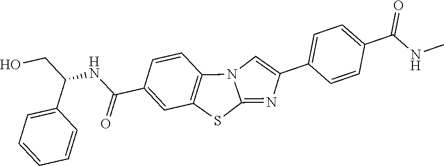 [SMILES: CNC(=O)c1ccc(-c2cn3c(n2)sc2cc(C(=O)N[C@@H](CO)c4ccccc4)ccc23)cc1]

Starting from 2-(4-(methylcarbamoyl)phenyl)benzo[d]imidazo[2,1-b]thiazole-7-carboxylic acid (100 mg, 0.285 mmol). Yield 21 mg (16%), as an off-white solid. ¹H NMR (400 MHz, DMSO) δ 8.96 (s, 1H), 8.86 (d, J=8.0 Hz, 1H), 8.58 (d, J=1.6 Hz, 1H), 8.48-8.46 (m, 1H), 8.13 (dd, J=1.6, 8.4 Hz, 1H), 8.08 (d, J=8.4 Hz, 1H), 7.96-7.94 (m, 2H), 7.92-7.90 (m, 2H), 7.47-7.40 (m, 2H), 7.35-7.33 (m, 2H), 7.30-7.21 (m, 1H), 5.11 (q, J=7.2 Hz, 1H), 4.99 (t, J=5.8 Hz, 1H), 3.80-3.63 (m, 2H), 2.81 (d, J=4.4 Hz, 3H). m/z: [ESI⁺] 471 (M+H)⁺, (C₂₆H₂₂N₄O₃S).

### (R)—N-(2-hydroxy-2-phenylethyl)-2-(4-(methylcarbamoyl)phenyl)benzo[d]imidazo[2,1-b]thiazole-7-carboxamide (Compound 256)

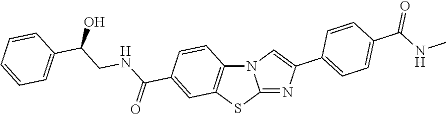 [SMILES: CNC(=O)c1ccc(-c2cn3c(n2)sc2cc(C(=O)NC[C@H](O)c4ccccc4)ccc23)cc1]

Starting from 2-(4-(methylcarbamoyl)phenyl)benzo[d]imidazo[2,1-b]thiazole-7-carboxylic acid (150 mg, 0.427 mmol). Yield 6 mg (3%), as a white solid. ¹H NMR (400 MHz, DMSO) δ 8.93 (s, 1H), 8.69 (t, J=5.6 Hz, 1H), 8.52 (s, 1H), 8.47-8.45 (m, 1H), 8.06 (s, 2H), 7.95 (d, J=8.6 Hz, 2H), 7.92 (d, J=8.6 Hz, 2H), 7.40 (d, J=7.6 Hz, 2H), 7.36 (dd, J=1.6, 7.6 Hz, 2H), 7.27 (dd, J=1.6, 7.2 Hz, 1H), 5.58 (s, 1H), 4.81 (t, J=6.2 Hz, 1H), 3.61-3.46 (m, 1H), 3.40-3.30 (m, 1H), 2.81 (d, J=4.4 Hz, 3H). m/z: [ESI⁺] 471 (M+H)⁺, (C₂₆H₂₂N₄O₃S).

### (R)—N-((1-ethylpyrrolidin-2-yl)methyl)-2-(4-(methylcarbamoyl)phenyl)benzo[d]imidazo[2,1-b]thiazole-7-carboxamide (Compound 214)

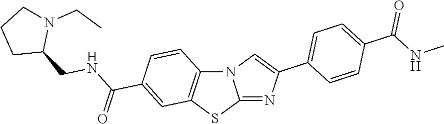 [SMILES: CCN1CCC[C@@H]1CNC(=O)c1ccc2c(c1)sc1nc(-c3ccc(C(=O)NC)cc3)cn12]

Starting from 2-(4-(methylcarbamoyl)phenyl)benzo[d]imidazo[2,1-b]thiazole-7-carboxylic acid (150 mg, 0.427 mmol). Yield 75 mg (38%), as a white solid. ¹H NMR (400 MHz, DMSO) δ 8.93 (s, 1H), 8.49 (d, J=1.6 Hz, 1H), 8.50-8.46 (m, 1H), 8.45-8.43 (m, 1H), 8.06 (d, J=8.4 Hz, 1H), 8.03 (dd, J=1.6, 8.4 Hz, 1H), 7.95 (d, J=8.6 Hz, 2H), 7.91 (d, J=8.6 Hz, 2H), 3.46 (td, J=4.6, 13.2 Hz, 1H), 3.19-2.99 (m, 2H), 2.91-2.83 (m, 1H), 2.81 (d, J=4.4 Hz, 3H), 2.65-2.56 (m, 1H), 2.31-2.23 (m, 1H), 2.14 (q, J=8.4 Hz, 1H), 1.83-1.81 (m, 1H), 1.71-1.57 (m, 3H), 1.06 (t, J=7.2 Hz, 3H). m/z: [ESI⁺]462 (M+H)⁺, (C₂₅H₂₇N₅O₂S).

### 2-(4-(Methylcarbamoyl)phenyl)-N-(2-(1-methylpyrrolidin-2-yl)ethyl)benzo[d]imidazo[2,1-b]thiazole-7-carboxamide hemiformate (Compound 257)

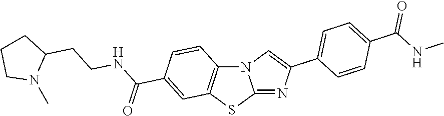 [SMILES: CNC(=O)c1ccc(-c2cn3c(n2)sc2cc(C(=O)NCCC4CCCN4C)ccc23)cc1]

Starting from 2-(4-(methylcarbamoyl)phenyl)benzo[d]imidazo[2,1-b]thiazole-7-carboxylic acid (150 mg, 0.427 mmol). Yield 23 mg (11%), as a white solid. ¹H NMR (400 MHz, DMSO) δ 8.93 (s, 1H), 8.64 (t, J=5.6 Hz, 1H), 8.49 (s, 1H), 8.47-8.45 (m, 1H), 8.06 (d, J=8.4 Hz, 1H), 8.02 (d, J=8.4 Hz, 1H), 7.95 (d, J=8.6 Hz, 2H), 7.91 (d, J=8.6 Hz, 2H), 3.33-3.26 (m, 2H), 3.04-2.93 (m, 1H), 2.80 (d, J=4.4 Hz, 3H), 2.26 (s, 3H), 2.18-2.09 (m, 2H), 2.03-1.88 (m, 2H), 1.65 (d, J=8.2 Hz, 2H), 1.47 (td, J=7.6, 14.0 Hz, 2H). m/z: [ESI⁺] 462 (M+H)⁺, (C₂₅H₂₇N₅O₂S).

### N-(1-(cyclopropanecarbonyl)piperidin-4-yl)-2-(4-(methylcarbamoyl)phenyl)benzo[d]imidazo[2,1-b]thiazole-7-carboxamide (Compound 190)

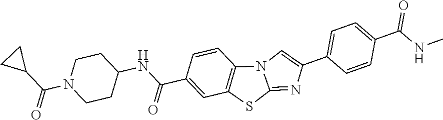 [SMILES: CNC(=O)c1ccc(-c2cn3c(n2)sc2cc(C(=O)NC4CCN(C(=O)C5CC5)CC4)ccc23)cc1]

Starting from 2-(4-(methylcarbamoyl)phenyl)benzo[d]imidazo[2,1-b]thiazole-7-carboxylic acid (101 mg, 0.287 mmol). Yield 32 mg (22%), as an off-white solid. ¹H NMR (400 MHz, DMSO) δ 8.94 (s, 1H), 8.51 (s, 1H), 8.50-8.42 (m, 2H), 8.06 (s, 2H), 7.95 (d, J=8.6 Hz, 2H), 7.91 (d, J=8.6 Hz, 2H), 4.40-4.28 (m, 2H), 4.12-4.10 (m, 1H), 3.30-3.25 (m, 1H), 2.81 (d, J=4.4 Hz, 3H), 2.68 (t, J=2.0 Hz, 1H), 2.02 (d, J=5.6 Hz, 1H), 2.00-1.80 (m, 2H), 1.60-1.35 (m, 2H), 0.80-0.68 (m, 4H). m/z: [ESI⁺]502 (M+H)⁺, (C₂₇H₂₇N₅O₃S).

### 2-(4-(Methylcarbamoyl)phenyl)-N-(1-(tetrahydro-2H-pyran-4-yl)ethyl)benzo[d]imidazo[2,1-b]thiazole-7-carboxamide (Compound 234)

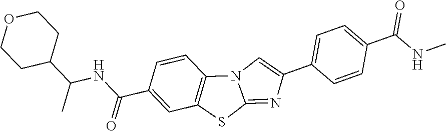 [SMILES: CNC(=O)c1ccc(-c2cn3c(n2)sc2cc(C(=O)NC(C)C4CCOCC4)ccc23)cc1]

Starting from 2-(4-(methylcarbamoyl)phenyl)benzo[d]imidazo[2,1-b]thiazole-7-carboxylic acid (200 mg, 0.569 mmol). Yield 72 mg (27%), as an off-white solid. ¹H NMR (400 MHz, DMSO) δ 8.94 (s, 1H), 8.50 (s, 1H), 8.47-8.65 (m, 1H), 8.30 (d, J=8.6 Hz, 1H), 8.05 (s, 2H), 7.95 (d, J=8.6 Hz, 2H), 7.91 (d, J=8.6 Hz, 2H), 3.95-3.84 m, 3H), 3.33-3.25 (m, 2H), 2.81 (d, J=4.4 Hz, 3H), 1.72-1.60 (m, 3H), 1.32-1.19 (m, 2H), 1.16 (d, J=6.8 Hz, 3H). m/z: [ESI⁺] 463 (M+H)⁺, (C₂₅H₂₆N₄O₃S).

### 2-(4-(Methylcarbamoyl)phenyl)-N-(3-oxo-3-(piperidin-1-yl)propyl)benzo[d]imidazo[2,1-b]thiazole-7-carboxamide (Compound 191)

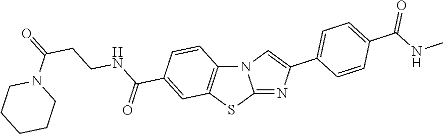 [SMILES: CNC(=O)c1ccc(-c2cn3c(n2)sc2cc(C(=O)NCCC(=O)N4CCCCC4)ccc23)cc1]

Starting from 2-(4-(methylcarbamoyl)phenyl)benzo[d]imidazo[2,1-b]thiazole-7-carboxylic acid (146 mg, 0.415 mmol). Yield 100 mg (49%), as an off-white solid. ¹H NMR (400 MHz, DMSO) δ 8.93 (s, 1H), 8.63 (t, J=5.6 Hz, 1H), 8.50 (s, 1H), 8.47-8.45 (m, 1H), 8.06 (d, J=8.4 Hz, 1H), 8.03 (dd, J=1.6, 8.4 Hz, 1H), 7.95 (d, J=8.6 Hz, 2H), 7.91 (d, J=8.6 Hz, 2H), 3.50 (q, J=6.8 Hz, 2H), 3.43 (td, J=5.6, 12.8 Hz, 3H), 2.81 (d, J=4.4 Hz, 3H), 2.63 (d, J=7.2 Hz, 2H), 1.57 (q, J=6.2 Hz, 2H), 1.50 (t, J=5.4 Hz, 2H), 1.42 (dd, J=4.0, 7.2 Hz, 2H). m/z: [ESI⁺] 490 (M+H)⁺, (C₂₆H₂₇N₅O₃S).

### (R)—N-(1-hydroxy-4-methylpentan-2-yl)-2-(4-(methylcarbamoyl)phenyl)benzo[d]imidazo[2,1-b]thiazole-7-carboxamide (Compound 192)

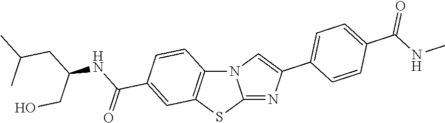 [SMILES: CNC(=O)c1ccc(-c2cn3c(n2)sc2cc(C(=O)N[C@@H](CO)CC(C)C)ccc23)cc1]

Starting from 2-(4-(methylcarbamoyl)phenyl)benzo[d]imidazo[2,1-b]thiazole-7-carboxylic acid (100 mg, 0.285 mmol). Yield 20 mg (15%), as an off-white solid. ¹H NMR (400 MHz, DMSO) δ 8.94 (s, 1H), 8.52 (d, J=1.2 Hz, 1H), 8.47-8.45 (m, 1H), 8.16 (d, J=8.6 Hz, 1H), 8.06 (s, 2H), 7.95 (d, J=8.6 Hz, 2H), 7.91 (d, J=8.6 Hz, 2H), 4.73 (t, J=5.8 Hz, 1H), 4.15-4.02 (m, 1H), 3.50-3.40 (m, 2H), 2.81 (d, J=4.4 Hz, 3H), 1.72-1.60 (m, 1H), 1.55-1.35 (m, 2H), 0.91 (t, J=7.2 Hz, 6H). m/z: [ESI⁺] 451 (M+H)⁺, (C₂₄H₂₆N₄O₃S).

### N-((1-ethylpiperidin-4-yl)methyl)-2-(4-(methylcarbamoyl)phenyl)benzo[d]imidazo[2,1-b]thiazole-7-carboxamide (Compound 215)

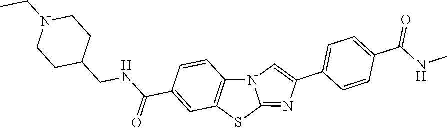 [SMILES: CCN1CCC(CNC(=O)c2ccc3c(c2)sc2nc(-c4ccc(C(=O)NC)cc4)cn23)CC1]

Starting from 2-(4-(methylcarbamoyl)phenyl)benzo[d]imidazo[2,1-b]thiazole-7-carboxylic acid (150 mg, 0.427 mmol). Yield 47 mg (23%), as an off-white solid. ¹H NMR (400 MHz, DMSO) δ 8.93 (s, 1H), 8.59 (t, J=5.8 Hz, 1H), 8.50 (d, J=1.2 Hz, 1H), 8.46-8.44 (m, 1H), 8.05 (s, 2H), 7.95 (d, J=8.6 Hz, 2H), 7.91 (d, J=8.6 Hz, 2H), 3.19 (t, J=6.4 Hz, 2H), 2.88 (d, J=11.2 Hz, 2H), 2.81 (d, J=4.4 Hz, 3H), 2.36-2.30 (m, 2H), 1.85 (t, J=11.6 Hz, 2H), 1.73-1.65 (m, 2H), 1.62-1.52 (m, 1H), 1.21-1.19 (m, 2H), 0.99 (t, J=7.2 Hz, 3H). m/z: [ESI⁺] 476 (M+H)⁺, (C₂₆H₂₉N₅O₂S).

### N-((1-(dimethylamino)cyclohexyl)methyl)-2-(4-(methylcarbamoyl)phenyl)benzo[d]imidazo[2,1-b]thiazole-7-carboxamide (Compound 216)

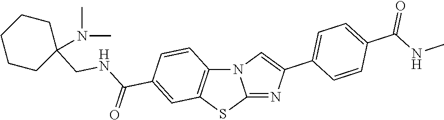 [SMILES: CNC(=O)c1ccc(-c2cn3c(n2)sc2cc(C(=O)NCC4(N(C)C)CCCCC4)ccc23)cc1]

Starting from 2-(4-(methylcarbamoyl)phenyl)benzo[d]imidazo[2,1-b]thiazole-7-carboxylic acid (100 mg, 0.285 mmol). Yield 14 mg (10%), as a white solid. ¹H NMR (400 MHz, DMSO) δ 8.95 (s, 1H), 8.51 (s, 1H), 8.47-8.45 (m, 1H), 8.20 (t, J=5.6 Hz, 1H), 8.06 (d, J=8.4 Hz, 1H), 8.03 (dd, J=1.6, 8.4 Hz, 1H), 7.95 (d, J=8.6 Hz, 2H), 7.91 (d, J=8.6 Hz, 2H), 3.30-3.26 (m, 2H), 2.81 (d, J=4.4 Hz, 3H), 2.31 (s, 6H), 1.80-1.68 (m, 2H), 1.60-1.42 (m, 3H), 1.40-1.20 (m, 5H). m/z: [ESI⁺] 490 (M+H)⁺, (C₂₇H₃₁N₅O₂S).

### N-(2-(diisopropylamino)ethyl)-2-(4-(methylcarbamoyl)phenyl)benzo[d]imidazo[2,1-b]thiazole-7-carboxamide (Compound 217)

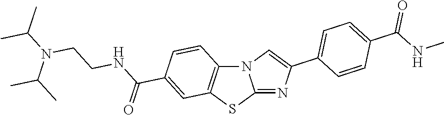 [SMILES: CNC(=O)c1ccc(-c2cn3c(n2)sc2cc(C(=O)NCCN(C(C)C)C(C)C)ccc23)cc1]

Starting from 2-(4-(methylcarbamoyl)phenyl)benzo[d]imidazo[2,1-b]thiazole-7-carboxylic acid (100 mg, 0.285 mmol). Yield 11 mg (8%), as a white solid. ¹H NMR (400 MHz, DMSO) δ 8.92 (s, 1H), 8.51 (t, J=5.6 Hz, 1H), 8.49 (s, 1H), 8.45-8.43 (m, 1H), 8.06 (d, J=8.4 Hz, 1H), 8.03 (dd, J=1.6, 8.4 Hz, 1H), 7.95 (d, J=8.6 Hz, 2H), 7.91 (d, J=8.6 Hz, 2H), 3.25 (dd, J=6.4, 8.8 Hz, 2H), 3.01-2.99 (m, 2H), 2.81 (d, J=4.4 Hz, 3H), 2.70-2.60 (m, 2H), 1.00 (d, J=6.4 Hz, 12H). m/z: [ESI⁺] 478 (M+H)⁺, (C₂₆H₃₁N₅O₂S).

### N-(3-hydroxy-2,2-dimethylcyclobutyl)-2-(4-(methylcarbamoyl)phenyl)benzo[d]imidazo[2,1-b]thiazole-7-carboxamide (Compound 258)

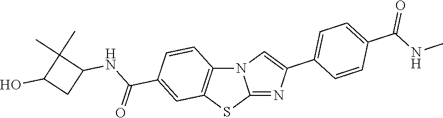 [SMILES: CNC(=O)c1ccc(-c2cn3c(n2)sc2cc(C(=O)NC4CC(O)C4(C)C)ccc23)cc1]

Starting from 2-(4-(methylcarbamoyl)phenyl)benzo[d]imidazo[2,1-b]thiazole-7-carboxylic acid (150 mg, 0.427 mmol). Yield 25 mg (13%), as an off-white solid. ¹H NMR (400 MHz, DMSO) δ 8.91 (s, 1H), 8.49 (s, 1H), 8.47-8.45 (m, 1H), 8.35 (d, J=7.2 Hz, 1H), 8.04 (s, 2H), 7.95 (d, J=8.6 Hz, 2H), 7.91 (d, J=8.6 Hz, 2H), 4.92 (s, 1H), 4.10 (td, J=6.2, 9.0 Hz, 1H), 3.85 (t, J=6.2 Hz, 1H), 2.80 (d, J=4.4 Hz, 3H), 2.42-2.28 (m, 1H), 2.15-1.97 (m, 1H), 1.34 (s, 1.5H), 1.07 (s, 1.5H), 0.91 (s, 1.5H), 0.88 (s, 1.5H). (a mixture of two cis/trans with a ratio of 1:1). m/z: [ESI⁺] 449 (M+H)⁺, (C₂₄H₂₄N₄O₃S).

### N-((2,2-difluorocyclopropyl)methyl)-2-(4-(methylcarbamoyl)phenyl)benzo[d]imidazo[2,1-b]thiazole-7-carboxamide (Compound 259)

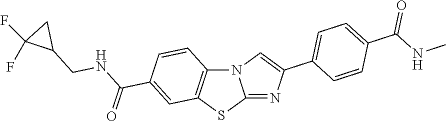 [SMILES: CNC(=O)c1ccc(-c2cn3c(n2)sc2cc(C(=O)NCC4CC4(F)F)ccc23)cc1]

Starting from 2-(4-(methylcarbamoyl)phenyl)benzo[d]imidazo[2,1-b]thiazole-7-carboxylic acid (150 mg, 0.427 mmol). Yield 28 mg (15%), as an off-white solid. ¹H NMR (400 MHz, DMSO) δ 8.93 (s, 1H), 8.89 (t, J=5.6 Hz, 1H), 8.53 (s, 1H), 8.46-8.44 (m, 1H), 8.07 (s, 2H), 7.95 (d, J=8.6 Hz, 2H), 7.91 (d, J=8.6 Hz, 2H), 3.43 (d, J=6.4 Hz, 2H), 2.81 (d, J=4.4 Hz, 3H), 2.12-1.96 (m, 1H), 1.63-1.61 (m, 1H), 1.37-1.35 (m, 1H). m/z: [ESI⁺] 441 (M+H)⁺, (C₂₂H₁₈F₂N₄O₂S).

### N-(((1r,4r)-4-hydroxycyclohexyl)methyl)-2-(4-(methylcarbamoyl)phenyl)benzo[d]imidazo[2,1-b]thiazole-7-carboxamide (Compound 273)

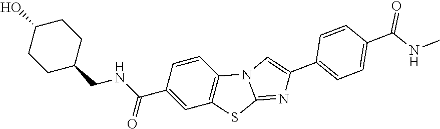 [SMILES: CNC(=O)c1ccc(-c2cn3c(n2)sc2cc(C(=O)NC[C@H]4CC[C@H](O)CC4)ccc23)cc1]

Starting from 2-(4-(methylcarbamoyl)phenyl)benzo[d]imidazo[2,1-b]thiazole-7-carboxylic acid (150 mg, 0.427 mmol). Yield 23 mg (12%), as an off-white solid. ¹H NMR (400 MHz, DMSO) δ 8.94 (d, J=2.0 Hz, 1H), 8.56 (t, J=5.6 Hz, 1H), 8.50 (s, 1H), 8.46-8.44 (m, 1H), 8.04 (s, 2H), 7.95 (d, J=8.6 Hz, 2H), 7.92 (d, J=8.6 Hz, 2H), 4.49 (s, 1H), 3.37-3.34 (m, 1H), 3.14 (t, J=6.4 Hz, 2H), 2.81 (d, J=4.4 Hz, 3H), 1.95-1.80 (m, 2H), 1.80-1.68 (m, 2H), 1.52-1.48 (m, 1H), 1.13-1.11 (m, 2H), 0.98-0.96 (m, 2H). m/z: [ESI⁺] 463 (M+H)⁺, (C₂₅H₂₆N₄O₃S).

### N-(2-(1-hydroxycyclopentyl)ethyl)-2-(4-(methylcarbamoyl)phenyl)benzo[d]imidazo[2,1-b]thiazole-7-carboxamide (Compound 260)

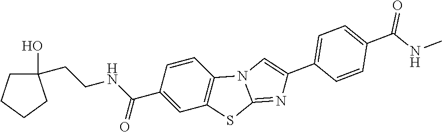 [SMILES: CNC(=O)c1ccc(-c2cn3c(n2)sc2cc(C(=O)NCCC4(O)CCCC4)ccc23)cc1]

Starting from 2-(4-(methylcarbamoyl)phenyl)benzo[d]imidazo[2,1-b]thiazole-7-carboxylic acid (150 mg, 0.427 mmol). Yield 13 mg (7%), as an off-white solid. ¹H NMR (400 MHz, DMSO) δ 8.92 (s, 1H), 8.54 (t, J=5.6 Hz, 1H), 8.48 (d, J=1.6 Hz, 1H), 8.46-8.44 (m, 1H), 8.06 (d, J=8.4 Hz, 1H), 8.03 (dd, J=1.6, 8.4 Hz, 1H), 7.95 (d, J=8.6 Hz, 2H), 7.91 (d, J=8.6 Hz, 2H), 4.23 (s, 1H), 3.49-3.39 (m, 2H), 2.81 (d, J=4.4 Hz, 3H), 1.84-1.77 (m, 2H), 1.77-1.69 (m, 2H), 1.69-1.54 (m, 2H), 1.58-1.43 (m, 4H). m/z: [ESI⁺] 463 (M+H)⁺, (C₂₅H₂₆N₄O₃S).

### 2-(4-(Methylcarbamoyl)phenyl)-N-((1-methylcyclopropyl)methyl)benzo[d]imidazo[2,1-b]thiazole-7-carboxamide (Compound 261)

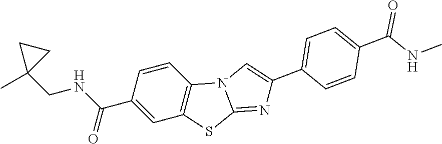

Starting from 2-(4-(methylcarbamoyl)phenyl)benzo[d]imidazo[2,1-b]thiazole-7-carboxylic acid (150 mg, 0.427 mmol). Yield 8 mg (5%), as an off-white solid. ¹H NMR (400 MHz, DMSO) δ 8.94 (s, 1H), 8.57 (t, J=6.0 Hz, 1H), 8.52 (dd, J=1.2, 1.6 Hz, 1H), 8.46-8.44 (m, 1H), 8.06 (s, 2H), 7.95 (d, J=8.6 Hz, 2H), 7.91 (d, J=8.6 Hz, 2H), 3.24 (d, J=6.0 Hz, 2H), 2.81 (d, J=4.4 Hz, 3H), 1.10 (s, 3H), 0.52 (dd, J=4.0, 5.6 Hz, 2H), 0.28 (dd, J=4.0, 5.6 Hz, 2H). m/z: [ESI⁺] 419 (M+H)⁺, (C₂₃H₂₂N₄O₂S).

### (R)—N-(1-cyclopropylethyl)-2-(4-(methylcarbamoyl)phenyl)benzo[d]imidazo[2,1-b]thiazole-7-carboxamide (Compound 274)

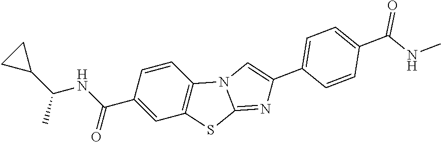 [SMILES: CNC(=O)c1ccc(-c2cn3c(n2)sc2cc(C(=O)N[C@H](C)C4CC4)ccc23)cc1]

Starting from 2-(4-(methylcarbamoyl)phenyl)benzo[d]imidazo[2,1-b]thiazole-7-carboxylic acid (150 mg, 0.427 mmol). Yield 38 mg (21%), as an off-white solid. ¹H NMR (400 MHz, DMSO) δ 8.94 (s, 1H), 8.51 (t, J=1.2 Hz, 1H), 8.46 (m, 2H), 8.06 (s, 2H), 7.95 (d, J=8.6 Hz, 2H), 7.92 (d, J=8.6 Hz, 2H), 3.53-3.51 (m, 1H), 2.81 (d, J=4.4 Hz, 3H), 1.26 (d, J=6.8 Hz, 3H), 1.03-1.01 (m, 1H), 0.49-0.47 (m, 1H), 0.44-0.37 (m, 1H), 0.35-0.33 (m, 1H), 0.24-0.22 (m, 1H). m/z: [ESI⁺] 419 (M+H)⁺, (C₂₃H₂₂N₄O₂S).

### 2-(4-(Methylcarbamoyl)phenyl)-N-(2-(2-methylpiperidin-1-yl)ethyl)benzo[d]imidazo[2,1-b]thiazole-7-carboxamide (Compound 262)

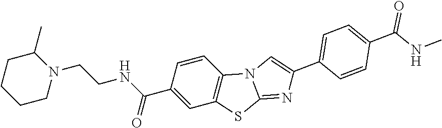 [SMILES: CNC(=O)c1ccc(-c2cn3c(n2)sc2cc(C(=O)NCCN4CCCCC4C)ccc23)cc1]

Starting from 2-(4-(methylcarbamoyl)phenyl)benzo[d]imidazo[2,1-b]thiazole-7-carboxylic acid (150 mg, 0.427 mmol). Yield 22 mg (11%), as an off-white solid. ¹H NMR (400 MHz, DMSO) δ 8.93 (s, 1H), 8.56 (t, J=5.6 Hz, 1H), 8.49 (d, J=1.6 Hz, 1H), 8.46-8.44 (m, 1H), 8.06 (d, J=8.4 Hz, 1H), 8.02 (dd, J=1.6, 8.4 Hz, 1H), 7.95 (d, J=8.6 Hz, 2H), 7.91 (d, J=8.6 Hz, 2H), 3.50-3.35 (m, 2H), 2.90-2.80 (m, 2H), 2.80 (d, J=4.4 Hz, 3H), 2.50-2.18 (m, 3H), 1.66-1.50 (m, 3H), 1.50-1.38 (m, 1H), 1.32-1.10 (m, 2H), 1.06 (d, J=6.2 Hz, 3H). m/z: [ESI⁺] 476 (M+H)⁺, (C₂₆H₂₉N₅O₂S).

### N-((1S,2R)-2-hydroxy-2,3-dihydro-1H-inden-1-yl)-2-(4-(methylcarbamoyl)phenyl)benzo[d]imidazo[2,1-b]thiazole-7-carboxamide hemiformate (Compound 263)

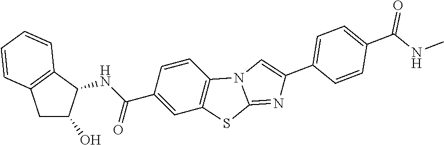 [SMILES: CNC(=O)c1ccc(-c2cn3c(n2)sc2cc(C(=O)N[C@H]4c5ccccc5C[C@H]4O)ccc23)cc1]

Starting from 2-(4-(methylcarbamoyl)phenyl)benzo[d]imidazo[2,1-b]thiazole-7-carboxylic acid (150 mg, 0.427 mmol). Yield 38 mg (18%), as an off-white solid. ¹H NMR (400 MHz, DMSO) δ 8.95 (s, 1H), 8.67 (d, J=1.6 Hz, 1H), 8.49-8.41 (m, 2H), 8.20 (dd, J=1.6, 8.4 Hz, 1H), 8.07 (d, J=8.4 Hz, 1H), 7.95 (d, J=8.6 Hz, 2H), 7.91 (d, J=8.6 Hz, 2H), 7.29 (dd, J=2.0, 6.8 Hz, 2H), 7.29-7.21 (m, 1H), 7.25-7.17 (m, 1H), 5.49 (dd, J=5.2, 8.6 Hz, 1H), 5.15 (d, J=4.4 Hz, 1H), 4.57-4.54 (m, 1H), 3.14 (dd, J=5.2, 16.2 Hz, 1H), 2.92 (dd, J=2.0, 16.2 Hz, 1H), 2.81 (d, J=4.4 Hz, 3H). m/z: [ESI⁺]483 (M+H)⁺, (C₂₇H₂₂N₄O₃S).

### 2-(4-(Methylcarbamoyl)phenyl)-N-(1-propylpiperidin-4-yl)benzo[d]imidazo[2,1-b]thiazole-7-carboxamide hemiformate (Compound 264)

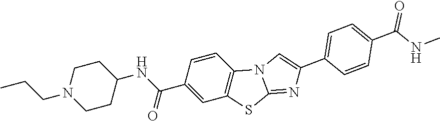 [SMILES: CCCN1CCC(NC(=O)c2ccc3c(c2)sc2nc(-c4ccc(C(=O)NC)cc4)cn23)CC1]

Starting from 2-(4-(methylcarbamoyl)phenyl)benzo[d]imidazo[2,1-b]thiazole-7-carboxylic acid (150 mg, 0.427 mmol). Yield 37 mg (18%), as a white solid. ¹H NMR (400 MHz, DMSO) δ 8.94 (s, 1H), 8.50 (d, J=1.2 Hz, 1H), 8.46-8.44 (m, 1H), 8.39 (d, J=7.6 Hz, 1H), 8.05 (s, 2H), 7.95 (d, J=8.6 Hz, 2H), 7.91 (d, J=8.6 Hz, 2H), 3.85-3.73 (m, 1H), 2.89 (d, J=11.2 Hz, 2H), 2.80 (d, J=4.4 Hz, 3H), 2.27 (dd, J=6.4, 8.4 Hz, 2H), 2.05-1.95 (m, 2H), 1.87-1.78 (m, 2H), 1.59 (dq, J=3.8, 12.4 Hz, 2H), 1.46-1.44 (m, 2H), 0.86 (t, J=7.2 Hz, 3H). m/z: [ESI⁺] 476 (M+H)⁺, (C₂₆H₂₉N₅O₂S).

### N-benzyl-2-(4-(methylcarbamoyl)phenyl)benzo[d]imidazo[2,1-b]thiazole-7-carboxamide (Compound 275)

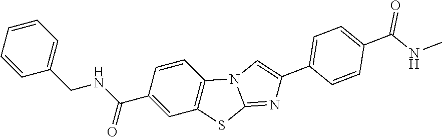 [SMILES: CNC(=O)c1ccc(-c2cn3c(n2)sc2cc(C(=O)NCc4ccccc4)ccc23)cc1]

Starting from 2-(4-(methylcarbamoyl)phenyl)benzo[d]imidazo[2,1-b]thiazole-7-carboxylic acid (300 mg, 0.854 mmol). Yield 6 mg (2%), as an off-white solid. ¹H NMR (400 MHz, DMSO) δ 9.18 (t, J=6.0 Hz, 1H), 8.94 (s, 1H), 8.57 (s, 1H), 8.46 (q, J=4.4 Hz, 1H), 8.10 (d, J=8.4 Hz, 1H), 8.07 (d, J=8.4 Hz, 1H), 7.95 (d, J=8.6 Hz, 2H), 7.92 (d, J=8.6 Hz, 2H), 7.42-7.31 (m, 4H), 7.27 (dd, J=2.4, 5.6 Hz, 1H), 4.53 (d, J=5.8 Hz, 2H), 2.81 (d, J=4.4 Hz, 3H). m/z: [ESI⁺] 441 (M+H)⁺, (C₂₅H₂₀N₄O₂S).

### N-(3-(5-methyl-2,5-diazabicyclo[2.2.1]heptan-2-yl)propyl)-2-(4-(methylcarbamoyl)phenyl)benzo[d]imidazo[2,1-b]thiazole-7-carboxamide formate (Compound 249)

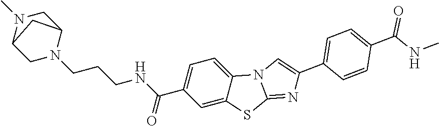

Starting from 2-(4-(methylcarbamoyl)phenyl)benzo[d]imidazo[2,1-b]thiazole-7-carboxylic acid (150 mg, 0.427 mmol). Yield 21 mg (9%), as a white solid. ¹H NMR (400 MHz, DMSO) δ 8.93 (s, 1H), 8.67 (t, J=5.6 Hz, 1H), 8.49 (d, J=1.6 Hz, 1H), 8.45 (d, J=4.4 Hz, 1H), 8.28 (s, 1H), 8.06 (d, J=8.4 Hz, 1H), 8.04 (dd, J=1.6, 8.4 Hz, 1H), 7.95 (d, J=8.6 Hz, 2H), 7.91 (d, J=8.6 Hz, 2H), 3.35-3.26 (m, 2H), 2.89 (s, 1H), 2.81 (d, J=4.4 Hz, 3H), 2.76-2.40 (m, 7H), 2.41 (s, 3H), 1.80-1.60 (m, 4H). m/z: [ESI⁺] 503 (M+H)⁺, (C₂₇H₃₀N₆O₂S).

### N-(3-(1H-imidazol-1-yl)propyl)-2-(4-(methylcarbamoyl)phenyl)benzo[d]imidazo[2,1-b]thiazole-7-carboxamide hemiformate (Compound 235)

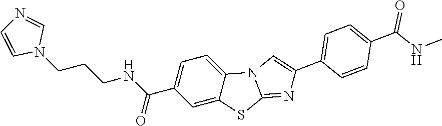 [SMILES: CNC(=O)c1ccc(-c2cn3c(n2)sc2cc(C(=O)NCCCn4ccnc4)ccc23)cc1]

Starting from 2-(4-(methylcarbamoyl)phenyl)benzo[d]imidazo[2,1-b]thiazole-7-carboxylic acid (150 mg, 0.427 mmol). Yield 23 mg (11%), as a white solid. ¹H NMR (400 MHz, DMSO) δ 8.94 (s, 1H), 8.65 (t, J=5.6 Hz, 1H), 8.51 (d, J=1.6 Hz, 1H), 8.45 (q, J=4.4 Hz, 1H), 8.07 (d, J=8.4 Hz, 1H), 8.04 (dd, J=1.6, 8.4 Hz, 1H), 7.95 (d, J=8.6 Hz, 2H), 7.91 (d, J=8.6 Hz, 2H), 7.74 (s, 1H), 7.26 (s, 1H), 6.94 (s, 1H), 4.07 (t, J=6.8 Hz, 2H), 3.33-3.27 (m, 2H), 2.81 (d, J=4.4 Hz, 3H), 2.00 (q, J=6.6 Hz, 2H). m/z: [ESI⁺] 459 (M+H)⁺, (C₂₄H₂₂N₆O₂S).

### N-(3-(5-methyl-2,5-diazabicyclo[2.2.1]heptan-2-yl)propyl)-2-(m-tolyl)benzo[d]imidazo[2,1-b]thiazole-7-carboxamide (Compound 248)

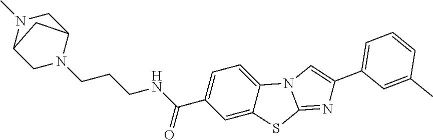

Starting from 2-(m-tolyl)benzo[d]imidazo[2,1-b]thiazole-7-carboxylic acid (100 mg, 0.324 mmol). Yield 10 mg (7%), as a white solid. ¹H NMR (400 MHz, DMSO) δ 8.80 (s, 1H), 8.68 (t, J=5.4 Hz, 1H), 8.47 (d, J=1.6 Hz, 1H), 8.05 (d, J=8.4 Hz, 1H), 8.01 (dd, J=1.6, 8.4 Hz, 1H), 7.71 (s, 1H), 7.67 (d, J=7.8 Hz, 1H), 7.33 (dd, J=1.6, 7.6 Hz, 1H), 7.12 (d, J=7.6 Hz, 1H), 3.25 (s, 1H), 3.09 (s, 1H), 2.62-2.52 (m, 4H), 2.48-2.40 (m, 4H), 2.44 (s, 3H), 2.24 (s, 3H), 1.64-1.62 (m, 2H), 1.55 (q, J=9.6 Hz, 2H). m/z: [ESI⁺] 460 (M+H)⁺, (C₂₆H₂₉N₅OS).

### 4-(Diethylamino)-N-(2-(m-tolyl)benzo[d]imidazo[2,1-b]thiazol-7-yl)butanamide (Compound 266)

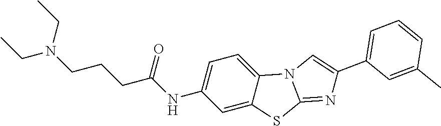 [SMILES: CCN(CC)CCCC(=O)Nc1ccc2c(c1)sc1nc(-c3cccc(C)c3)cn12]

Starting from 2-(m-tolyl)benzo[d]imidazo[2,1-b]thiazol-7-aminium chloride (150 mg, 0.475 mmol) and 4-(diethylamino)butanoic acid (103 mg, 0.647 mmol). Yield 12 mg (5%), as an off-white solid. ¹H NMR (400 MHz, DMSO) δ 10.19 (s, 1H), 8.68 (s, 1H), 8.32 (d, J=2.0 Hz, 1H), 7.89 (d, J=8.6 Hz, 1H), 7.70 (s, 1H), 7.68-7.60 (m, 2H), 7.31 (dd, J=1.6, 7.6 Hz, 1H), 7.10 (d, J=7.6 Hz, 1H), 2.49-2.35 (m, 8H), 2.37 (s, 3H), 1.73-1.71 (m, 2H), 0.95 (t, J=7.2 Hz, 6H). m/z: [ESI⁺] 421 (M+H)⁺, (C₂₄H₂₈N₄OS).

### General Procedure D for De-Boc

A solution of the corresponding substrates (1.00 eq.) in a 4M solution of HCl in dioxane (0.10M) was stirred for 2-16 h at room temperature under a nitrogen atmosphere. Upon completion, the resulting mixture was concentrated under reduced pressure and purified by reverse phase flash chromatography with the addition of NH₄HCO₃ will produce the parent product while with the addition of formic acid, will produce the product as formate form. The fractions containing desired product were collected, concentrated under reduced pressure and lyophilized to produce the corresponding products.

### N-(piperidin-4-yl)-2-(m-tolyl)benzo[d]imidazo[2,1-b]thiazole-7-carboxamide (Compound 140)

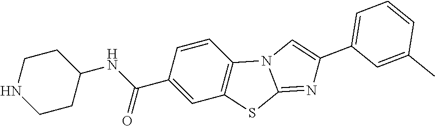 [SMILES: Cc1cccc(-c2cn3c(n2)sc2cc(C(=O)NC4CCNCC4)ccc23)c1]

Starting from tert-butyl 4-(2-(m-tolyl)benzo[d]imidazo[2,1-b]thiazole-7-carboxamido)piperidine-1-carboxylate (0.41 g, 0.84 mmol); Yield 0.17 g (52%), as an off-white solid. ¹H NMR (400 MHz, DMSO) δ 8.81 (s, 1H), 8.49 (s, 1H), 8.39 (d, J=7.8 Hz, 1H), 8.04 (s, 2H), 7.71 (s, 1H), 7.66 (d, J=7.8 Hz, 1H), 7.33 (t, J=7.6 Hz, 1H), 7.12 (d, J=7.4 Hz, 1H), 3.92-3.80 (m, 1H), 3.03-2.93 (m, 2H), 2.60-2.50 (m, 2H), 2.37 (s, 3H), 1.82-1.72 (m, 2H), 1.44 (dq, J=4.0, 11.8 Hz, 2H). m/z: [ESI⁺] 391 (M+H)⁺, (C₂₂H₂₂N₄OS).

### Piperazin-1-yl(2-(m-tolyl)benzo[d]imidazo[2,1-b]thiazol-7-yl)methanone (Compound 141)

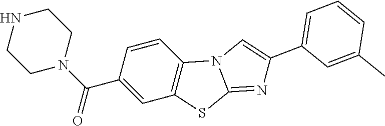 [SMILES: Cc1cccc(-c2cn3c(n2)sc2cc(C(=O)N4CCNCC4)ccc23)c1]

Starting from tert-butyl 4-(2-(m-tolyl)benzo[d]imidazo[2,1-b]thiazole-7-carbonyl)piperazine-1-carboxylate (0.42 g, 0.88 mmol); Yield 0.23 g (69%), as an off-white solid. ¹H NMR (400 MHz, DMSO) δ 8.80 (s, 1H), 8.12 (d, J=1.6 Hz, 1H), 8.01 (d, J=8.4 Hz, 1H), 7.71 (s, 1H), 7.67 (d, J=7.8 Hz, 1H), 7.58 (dd, J=1.6, 8.4 Hz, 1H), 7.33 (dd, J=1.6, 7.6 Hz, 1H), 7.12 (d, J=7.6 Hz, 1H), 3.55 (s, 2H), 3.33 (s, 2H), 2.71 (s, 4H), 2.37 (s, 3H). m/z: [ESI⁺] 377 (M+H)⁺, (C₂₁H₂₀N₄OS).

### N-(2-(pyrrolidin-2-yl)ethyl)-2-(m-tolyl)benzo[d]imidazo[2,1-b]thiazole-7-carboxamide (Compound 156)

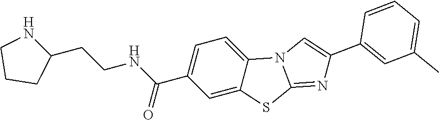 [SMILES: Cc1cccc(-c2cn3c(n2)sc2cc(C(=O)NCCC4CCCN4)ccc23)c1]

Starting from tert-butyl 2-(2-(2-(m-tolyl)benzo[d]imidazo[2,1-b]thiazole-7-carboxamido)ethyl)pyrrolidine-1-carboxylate (200 mg 0.396 mmol). Yield 45 mg (28%), as a white solid. ¹H NMR (400 MHz, DMSO) δ 8.81 (s, 1H), 8.77 (t, J=5.6 Hz, 1H), 8.50 (s, 1H), 8.06-8.02 (m, 2H), 7.72 (s, 1H), 7.67 (d, J=7.8 Hz, 1H), 7.33 (dd, J=1.6, 7.6 Hz, 1H), 7.13 (d, J=7.6 Hz, 1H), 3.34-3.26 (m, 2H), 3.10-3.08 (m, 1H), 2.94-2.90 (m, 1H), 2.84-2.80 (m, 1H), 2.38 (s, 3H), 1.98-1.86 (m, 1H), 1.81-1.51 (m, 4H), 1.29 (qd, J=8.4, 12.2 Hz, 1H). m/z: [ESI⁺] 405 (M+H)⁺, (C₂₃H₂₄N₄OS).

### N-(3-(ethylamino)propyl)-2-(m-tolyl)benzo[d]imidazo[2,1-b]thiazole-7-carboxamide (Compound 155)

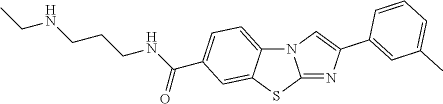 [SMILES: CCNCCCNC(=O)c1ccc2c(c1)sc1nc(-c3cccc(C)c3)cn12]

Starting from tert-butyl ethyl(3-(2-(m-tolyl)benzo[d]imidazo[2,1-b]thiazole-7-carboxamido)propyl) carbamate (400 mg, 0.812 mmol). Yield 120 mg (38%), as an off-white solid: ¹H NMR (400 MHz, DMSO) δ 8.81 (d, J=1.2 Hz, 1H), 8.73 (t, J=5.6 Hz, 1H), 8.49 (d, J=1.6 Hz, 1H), 8.05 (d, J=8.4 Hz, 1H), 8.02 (dd, J=1.6, 8.4 Hz, 1H), 7.72 (s, 1H), 7.67 (d, J=7.8 Hz, 1H), 7.33 (dd, J=1.6, 7.6 Hz, 1H), 7.13 (d, J=7.6 Hz, 1H), 3.38-3.34 (m, 2H), 2.64-2.53 (m, 4H), 2.38 (s, 3H), 1.70-1.68 (m, 2H), 1.03 (t, J=7.2 Hz, 3H). m/z: [ESI⁺] 393 (M+H)⁺, (C₂₂H₂₄N₄OS).

### 2-(4-(Methylcarbamoyl)phenyl)-N-(3-(piperazin-1-yl)propyl)benzo[d]imidazo[2,1-b]thiazole-7-carboxamide (Compound 175)

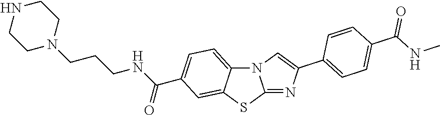 [SMILES: CNC(=O)c1ccc(-c2cn3c(n2)sc2cc(C(=O)NCCCN4CCNCC4)ccc23)cc1]

Starting from tert-butyl 4-(3-(2-(4-(methylcarbamoyl)phenyl)benzo[d] imidazo[2,1-b]thiazole-7-carboxamido)propyl)piperazine-1-carboxylate (100 mg, 0.173 mmol). Yield 21 mg (25%), as a white solid. ¹H NMR (400 MHz, DMSO) δ 8.94 (s, 1H), 8.63 (t, J=5.6 Hz, 1H), 8.50 (d, J=1.6 Hz, 1H), 8.46 (q, J=4.4 Hz, 1H), 8.05 (d, J=8.4 Hz, 1H), 8.02 (dd, J=1.6, 8.4 Hz, 1H), 7.95 (d, J=8.6 Hz, 2H), 7.91 (d, J=8.6 Hz, 2H), 3.31-3.26 (m, 2H), 2.81 (d, J=4.4 Hz, 3H), 2.72 (t, J=4.8 Hz, 4H), 2.40-2.36 (m, 6H), 1.72-1.70 (m, 2H). m/z: [ESI⁺] 477 (M+H)⁺, (C₂₅H₂₈N₆O₂S).

### 2-(m-Tolyl)-N-(3-((2,2,2-trifluoroethyl)amino)propyl)benzo[d]imidazo[2,1-b]thiazole-7-carboxamide (Compound 178)

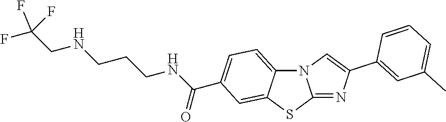 [SMILES: Cc1cccc(-c2cn3c(n2)sc2cc(C(=O)NCCCNCC(F)(F)F)ccc23)c1]

Starting from tert-butyl (3-(2-(m-tolyl)benzo[d]imidazo[2,1-b]thiazole-7-carboxamido)propyl)(2,2,2-trifluoroethyl) carbamate (400 mg, 0.732 mmol). Yield 180 mg (55%), as an off-white solid. ¹H NMR (400 MHz, DMSO) δ 8.81 (s, 1H), 8.60 (t, J=5.6 Hz, 1H), 8.48 (d, J=1.6 Hz, 1H), 8.05 (d, J=8.4 Hz, 1H), 8.02 (dd, J=1.6, 8.4 Hz, 1H), 7.72 (d, J=1.8 Hz, 1H), 7.67 (d, J=7.8 Hz, 1H), 7.33 (dd, J=1.6, 7.6 Hz, 1H), 7.13 (d, J=7.5 Hz, 1H), 3.36 (t, J=7.0 Hz, 2H), 3.24 (q, J=10.2 Hz, 2H), 2.68 (t, J=7.0 Hz, 2H), 2.38 (s, 3H), 1.70-1.68 (m, 2H). m/z: [ESI⁺] 447 (M+H)⁺, (C₂₂H₂₁F₃N₄OS).

### 2-(4-(Aminomethyl)phenyl)-N-(3-(diethylamino)propyl)benzo[d]imidazo[2,1-b]thiazole-7-carboxamide (Compound 163)

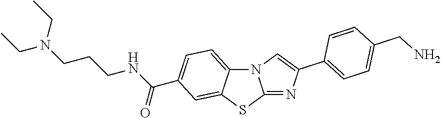 [SMILES: CCN(CC)CCCNC(=O)c1ccc2c(c1)sc1nc(-c3ccc(CN)cc3)cn12]

Starting from tert-butyl (4-(7-((3-(diethylamino)propyl)carbamoyl)benzo[d] imidazo[2,1-b]thiazol-2-yl)benzyl)carbamate (1.00 g, 1.87 mmol). Yield 98 mg (12%), as an off-white solid. ¹H NMR (400 MHz, DMSO) δ 8.83 (s, 1H), 8.65 (t, J=6.0 Hz, 1H), 8.49 (d, J=1.6 Hz, 1H), 8.34 (s, 1H), 8.05 (d, J=8.4 Hz, 1H), 8.02 (dd, J=1.6, 8.4 Hz, 1H), 7.88 (d, J=8.0 Hz, 2H), 7.50 (d, J=8.0 Hz, 2H), 3.96 (s, 2H), 3.36-3.30 (m, 2H), 2.50-2.46 (m, 6H), 1.70 (p, J=7.2 Hz, 2H), 0.98 (t, J=7.2 Hz, 6H). m/z: [ESI⁺] 436 (M+H)⁺, (C₂₄H₂₉N₅OS).

### N-(2-(m-tolyl)benzo[d]imidazo[2,1-b]thiazol-7-yl)piperidine-4-carboxamide (Compound 168)

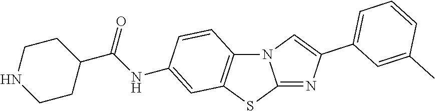 [SMILES: Cc1cccc(-c2cn3c(n2)sc2cc(NC(=O)C4CCNCC4)ccc23)c1]

Starting from tert-butyl 4-((2-(m-tolyl)benzo[d]imidazo[2,1-b]thiazol-7-yl)carbamoyl)piperidine-1-carboxylate (150 mg, 0.306 mmol). Yield 30 mg (25%), as a white solid. ¹H NMR (400 MHz, DMSO) δ 10.13 (br s, 1H), 8.69 (s, 1H), 8.33 (d, J=2.0 Hz, 1H), 7.89 (d, J=8.8 Hz, 1H), 7.70 (d, J=2.0 Hz, 1H), 7.68-7.62 (m, 2H), 7.31 (dd, J=1.6, 7.6 Hz, 1H), 7.10 (d, J=7.2 Hz, 1H), 3.04-2.96 (m, 2H), 2.55-2.53 (m, 1H), 2.49-2.41 (m, 2H), 2.37 (s, 3H), 1.71 (d, J=12.4 Hz, 2H), 1.54 (dq, J=4.0, 12.2 Hz, 2H). m/z: [ESI⁺] 391 (M+H)⁺, (C₂₂H₂₂N₄OS).

### N-(azetidin-3-ylmethyl)-2-(p-tolyl)benzo[d]imidazo[2,1-b]thiazole-7-carboxamide (Compound 117)

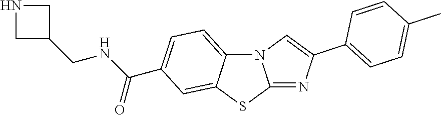 [SMILES: Cc1ccc(-c2cn3c(n2)sc2cc(C(=O)NCC4CNC4)ccc23)cc1]

Starting from tert-butyl 3-((2-(p-tolyl)benzo[d]imidazo[2,1-b]thiazole-7-carboxamido)methyl) azetidine-1-carboxylate (0.35 g, 0.73 mmol). Yield 18 mg (7%), as a white solid. ¹H NMR (400 MHz, DMSO) δ 8.71 (d, J=1.6 Hz, 1H), 8.37 (s, 1H), 7.96 (d, J=8.4 Hz, 1H), 7.91 (dd, J=1.6, 8.4 Hz, 1H), 7.76 (d, J=8.0 Hz, 2H), 7.25 (d, J=8.0 Hz, 2H), 4.61 (br s, 1H), 3.48 (dd, J=4.4, 13.2 Hz, 2H), 3.40 (d, J=6.4 Hz, 2H), 3.11 (dd, J=8.6, 13.6 Hz, 2H), 2.34 (s, 3H), 1.83 (s, 1H). m/z: [ESI⁺] 377 (M+H)⁺, (C₂₁H₂₀N₄OS).

### (S)—N-(1-aminopropan-2-yl)-2-(4-(methylcarbamoyl)phenyl)benzo[d]imidazo[2,1-b]thiazole-7-carboxamide formate (Compound 220)

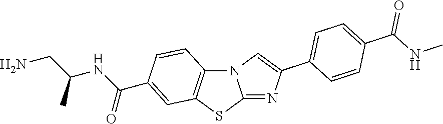 [SMILES: CNC(=O)c1ccc(-c2cn3c(n2)sc2cc(C(=O)N[C@@H](C)CN)ccc23)cc1]

Starting from tert-butyl (S)-(2-(2-(4-(methylcarbamoyl)phenyl)benzo[d] imidazo[2,1-b]thiazole-7-carboxamido)propyl)carbamate (150 mg, 0.296 mmol). Yield 30 mg (25%), as a white solid. ¹H NMR (400 MHz, DMSO) δ 8.94 (s, 1H), 8.62 (d, J=7.8 Hz, 1H), 8.56 (d, J=1.6 Hz, 1H), 8.45 (q, J=4.4 Hz, 1H), 8.36 (s, 1H), 8.10 (dd, J=1.6, 8.4 Hz, 1H), 8.06 (d, J=8.4 Hz, 1H), 7.95 (d, J=8.6 Hz, 2H), 7.92 (d, J=8.6 Hz, 2H), 4.19 (q, J=6.6 Hz, 1H), 2.87 (d, J=6.4 Hz, 2H), 2.81 (d, J=4.4 Hz, 3H), 1.21 (d, J=6.6 Hz, 3H). m/z: [ESI⁺] 408 (M+H)⁺, (C₂₂H₂₃N₅O₄S).

### N-((1-aminocyclopropyl)methyl)-2-(4-(methylcarbamoyl)phenyl)benzo[d]imidazo[2,1-b]thiazole-7-carboxamide formate (Compound 251)

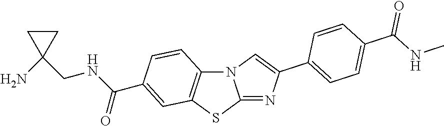 [SMILES: CNC(=O)c1ccc(-c2cn3c(n2)sc2cc(C(=O)NCC4(N)CC4)ccc23)cc1]

Starting from tert-butyl (1-((2-(4-(methylcarbamoyl)phenyl)benzo[d] imidazo[2,1-b]thiazole-7-carboxamido)methyl)cyclopropyl)carbamate (150 mg, 0.289 mmol). Yield 10 mg (8%), as an off-white sold. ¹H NMR (400 MHz, DMSO) δ 8.93 (s, 1H), 8.81 (t, J=5.6 Hz, 1H), 8.56 (d, J=1.6 Hz, 1H), 8.46 (q, J=4.4 Hz, 1H), 8.05 (d, J=8.4 Hz, 1H), 8.02 (dd, J=1.6, 8.4 Hz, 1H), 7.95 (d, J=8.6 Hz, 2H), 7.91 (d, J=8.6 Hz, 2H), 3.43 (d, J=5.6 Hz, 2H), 2.81 (d, J=4.4 Hz, 3H), 0.72-0.59 (m, 4H). m/z: [ESI⁺] 420 (M+H)⁺, (C₂₂H₂₁N₅O₂S).

### N-(((3R,4R)-3-hydroxypiperidin-4-yl)methyl)-2-(4-(methylcarbamoyl)phenyl)benzo[d]imidazo[2,1-b]thiazole-7-carboxamide formate (Compound 196)

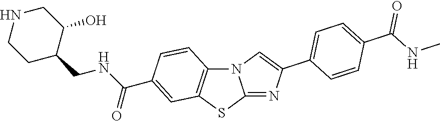 [SMILES: CNC(=O)c1ccc(-c2cn3c(n2)sc2cc(C(=O)NC[C@H]4CCNC[C@@H]4O)ccc23)cc1]

Starting from tert-butyl (3R,4R)-3-hydroxy-4-((2-(4-(methylcarbamoyl)phenyl)benzo[d]imidazo[2,1-b]thiazole-7-carboxamido)methyl) piperidine-1-carboxylate (150 mg, 0.266 mmol). Yield 27 mg (20%), as a white solid. ¹H NMR (400 MHz, DMSO) δ 8.94 (s, 1H), 8.59 (s, 1H), 8.52 (s, 1H), 8.45 (q, J=4.4 Hz, 1H), 8.32 (s, 1H), 8.06 (s, 2H), 7.95 (d, J=8.6 Hz, 2H), 7.91 (d, J=8.6 Hz, 2H), 3.61 (d, J=11.8 Hz, 1H), 3.36 (s, 1H), 3.28 (dt, J=7.2, 14.0 Hz, 1H), 3.11 (d, J=11.4 Hz, 1H), 3.02 (d, J=12.2 Hz, 1H), 2.81 (d, J=4.4 Hz, 3H), 2.55-2.50 (m, 1H), 2.44 (d, J=10.6 Hz, 1H), 1.82 (d, J=13.4 Hz, 1H), 1.64 (s, 1H), 1.28 (s, 1H). m/z: [ESI⁺] 464 (M+H)⁺, (C₂₄H₂₅N₅O₃S).

### N-((1R,5S,6s)-3-azabicyclo[3.1.0]hexan-6-yl)-2-(4-(methylcarbamoyl)phenyl)benzo[d]imidazo[2,1-b]thiazole-7-carboxamide formate (Compound 253)

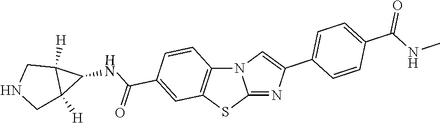 [SMILES: CNC(=O)c1ccc(-c2cn3c(n2)sc2cc(C(=O)N[C@H]4[C@@H]5CNC[C@@H]54)ccc23)cc1]

Starting from tert-butyl (1R,5S,6s)-6-(2-(4-(methylcarbamoyl)phenyl)benzo[d]imidazo[2,1-b]thiazole-7-carboxamido)-3-azabicyclo[3.1.0]hexane-3-carboxylate (100 mg, 0.188 mmol). Yield 11 mg (12%), as an off-white sold. ¹H NMR (400 MHz, DMSO) δ 8.97-8.89 (m, 1H), 8.61 (d, J=11.8 Hz, 1H), 8.51-8.43 (m, 2H), 8.07-7.98 (m, 2H), 7.95 (d, J=8.6 Hz, 2H), 7.91 (d, J=8.6 Hz, 2H), 3.15 (s, 2H), 3.05-2.92 (m, 2H), 2.86 (s, 1H), 2.81 (d, J=4.0 Hz, 3H), 1.78 (d, J=12.4 Hz, 2H). m/z: [ESI⁺] 432 (M+H)⁺, (C₂₃H₂₁N₅O₂S).

### N-(1-(aminomethyl)cyclobutyl)-2-(4-(methylcarbamoyl)phenyl)benzo[d]imidazo[2,1-b]thiazole-7-carboxamide (Compound 199)

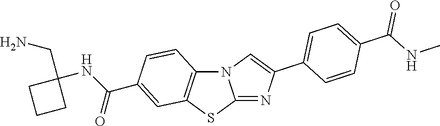 [SMILES: CNC(=O)c1ccc(-c2cn3c(n2)sc2cc(C(=O)NC4(CN)CCC4)ccc23)cc1]

Starting from tert-butyl ((1-(2-(4-(methylcarbamoyl)phenyl)benzo[d]imidazo[2,1-b]thiazole-7-carboxamido)cyclobutyl)methyl)carbamate (130 mg, 0.244 mmol). Yield 24 mg (23%), as a white solid. ¹H NMR (400 MHz, DMSO) δ 8.93 (s, 1H), 8.92 (s, 1H), 8.51 (d, J=1.6 Hz, 1H), 8.44 (q, J=4.4 Hz, 1H), 8.39 (d, J=7.4 Hz, 1H), 8.06 (dd, J=1.6, 8.4 Hz, 1H), 8.03 (d, J=8.4 Hz, 1H), 7.95 (d, J=8.6 Hz, 2H), 7.92 (d, J=8.6 Hz, 2H), 2.94 (s, 1H), 2.81 (d, J=4.4 Hz, 3H), 2.31 (dq, J=5.2, 9.6 Hz, 2H), 2.18-2.05 (m, 2H), 1.91-1.67 (m, 2H). m/z: [ESI⁺] 434 (M+H)⁺, (C₂₃H₂₃N₅O₂S).

### N-((1R,4R,5S)-2-azabicyclo[2.1.1]hexan-5-yl)-2-(4-(methylcarbamoyl)phenyl)benzo[d]imidazo[2,1-b]thiazole-7-carboxamide (Compound 238)

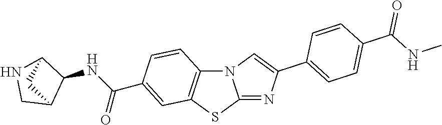 [SMILES: CNC(=O)c1ccc(-c2cn3c(n2)sc2cc(C(=O)N[C@H]4[C@H]5CN[C@@H]4C5)ccc23)cc1]

Starting from tert-butyl (1R,4R,5S)-5-(2-(4-(methylcarbamoyl)phenyl)benzo[d]imidazo[2,1-b]thiazole-7-carboxamido)-2-azabicyclo[2.1.1]hexane-2-carboxylate (150 mg, 0.282 mmol). Yield 31 mg (25%), as an off-white solid. ¹H NMR (400 MHz, DMSO) δ 8.92 (s, 1H), 8.50 (s, 1H), 8.45 (q, J=4.4 Hz, 1H), 8.13-8.07 (m, 1H), 8.03 (s, 2H), 7.95 (d, J=8.6 Hz, 2H), 7.91 (d, J=8.6 Hz, 2H), 3.71 (s, 1H), 3.59 (d, J=5.2 Hz, 1H), 2.85-2.81 (m, 6H), 1.44 (d, J=7.2 Hz, 1H), 1.04 (d, J=7.2 Hz, 1H). m/z: [ESI⁺] 432 (M+H)⁺, (C₂₃H₂₁N₅O₂S).

### (S)—N-(3-aminobutyl)-2-(4-(methylcarbamoyl)phenyl)benzo[d]imidazo[2,1-b]thiazole-7-carboxamide (Compound 200)

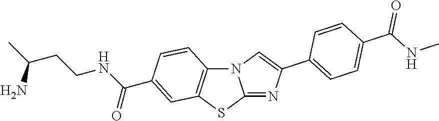 [SMILES: CNC(=O)c1ccc(-c2cn3c(n2)sc2cc(C(=O)NCC[C@H](C)N)ccc23)cc1]

Starting from tert-butyl (S)-(4-(2-(4-(methylcarbamoyl)phenyl)benzo[d]imidazo[2,1-b]thiazole-7-carboxamido)butan-2-yl)carbamate (150 mg, 0.288 mmol). Yield 32 mg (26%), as an off-white solid. ¹H NMR (400 MHz, DMSO) δ 8.93 (s, 1H), 8.72 (t, J=5.6 Hz, 1H), 8.49 (s, 1H), 8.46 (q, J=4.4 Hz, 1H), 8.10-7.99 (m, 2H), 7.95 (d, J=8.6 Hz, 2H), 7.91 (d, J=8.6 Hz, 2H), 3.45-3.35 (m, 2H), 2.92-2.84 (m, 1H), 2.81 (d, J=4.4 Hz, 4H), 1.66-1.40 (m, 2H), 1.03 (d, J=6.4 Hz, 3H). m/z: [ESI⁺] 422 (M+H)⁺, (C₂₂H₂₃N₅O₂S).

### (S)-2-(4-(methylcarbamoyl)phenyl)-N-(pyrrolidin-3-yl)benzo[d]imidazo[2,1-b]thiazole-7-carboxamide (Compound 222)

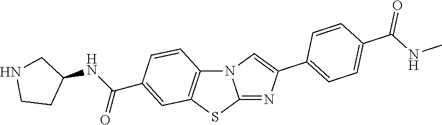 [SMILES: CNC(=O)c1ccc(-c2cn3c(n2)sc2cc(C(=O)N[C@H]4CCNC4)ccc23)cc1]

Starting from tert-butyl (S)-3-(2-(4-(methylcarbamoyl)phenyl)benzo[d]imidazo[2,1-b]thiazole-7-carboxamido)pyrrolidine-1-carboxylate (110 mg, 0.212 mmol). Yield 31 mg (35%), as a white solid. ¹H NMR (400 MHz, DMSO) δ 8.93 (s, 1H), 8.69 (d, J=6.8 Hz, 1H), 8.53 (d, J=1.6 Hz, 1H), 8.45 (q, J=4.4 Hz, 1H), 8.06 (dd, J=1.6, 8.4 Hz, 1H), 8.03 (d, J=8.4 Hz, 1H), 7.95 (d, J=8.6 Hz, 2H), 7.91 (d, J=8.6 Hz, 2H), 4.52-4.35 (m, 1H), 3.62-3.50 (m, 1H), 3.10-2.95 (m, 1H), 2.90-2.73 (m, 1H), 2.81 (d, J=4.4 Hz, 3H), 2.14 (s, 1H), 2.10-1.90 (m, 1H), 1.76 (s, 1H). m/z: [ESI⁺] 420 (M+H)⁺, (C₂₂H₂₁N₅O₂S).

### (R)-2-(4-(methylcarbamoyl)phenyl)-N-(pyrrolidin-2-ylmethyl)benzo[d]imidazo[2,1-b]thiazole-7-carboxamide formate (Compound 224)

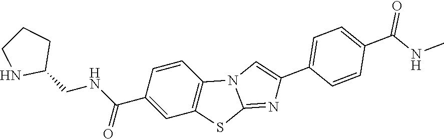 [SMILES: CNC(=O)c1ccc(-c2cn3c(n2)sc2cc(C(=O)NC[C@H]4CCCN4)ccc23)cc1]

Starting from tert-butyl (R)-2-((2-(4-(methylcarbamoyl)phenyl) benzo[d]imidazo[2,1-b]thiazole-7-carboxamido)methyl)pyrrolidine-1-carboxylate (150 mg, 0.281 mmol). Yield: 87 mg (65%), as an off-white solid. ¹H NMR (400 MHz, DMSO) δ 9.43 (s, 1H), 8.91 (s, 1H), 8.56 (s, 1H), 8.47 (q, J=4.4 Hz, 1H), 8.46 (s, 1H), 8.11 (d, J=8.6 Hz, 1H), 8.05 (d, J=8.6 Hz, 1H), 7.95 (d, J=8.6 Hz, 2H), 7.91 (d, J=8.6 Hz, 2H), 3.59 (dd, J=4.8, 14.2 Hz, 2H), 3.46 (d, J=10.0 Hz, 1H), 3.12 (t, J=6.8 Hz, 1H), 3.08 (t, J=6.8 Hz, 1H), 2.81 (d, J=4.4 Hz, 3H), 2.01-1.99 (m, 1H), 1.90-1.73 (m, 2H), 1.60 (dq, J=7.8, 16.0 Hz, 1H). m/z: [ESI⁺] 434 (M+H)⁺, (C₂₃H₂₃N₅O₂S).

### 2-(4-(Methylcarbamoyl)phenyl)-N-(2-azaspiro[3.3]heptan-6-yl)benzo[d]imidazo[2,1-b]thiazole-7-carboxamide formate (Compound 241)

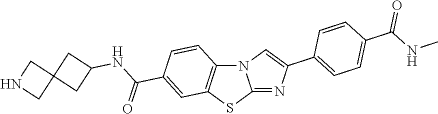 [SMILES: CNC(=O)c1ccc(-c2cn3c(n2)sc2cc(C(=O)NC4CC5(CNC5)C4)ccc23)cc1]

Starting from tert-butyl 6-(2-(4-(methylcarbamoyl)phenyl) benzo[d]imidazo[2,1-b]thiazole-7-carboxamido)-2-azaspiro[3.3]heptane-2-carboxylate (120 mg, 0.220 mmol). Yield 14 mg (13%), as a white solid. ¹H NMR (400 MHz, DMSO) δ 8.93 (s, 1H), 8.76 (d, J=7.2 Hz, 1H), 8.49 (s, 1H), 8.46 (q, J=4.4 Hz, 1H), 8.44 (s, 1H), 8.06 (d, J=8.6 Hz, 1H), 8.03 (d, J=8.6 Hz, 1H), 7.95 (d, J=8.6 Hz, 2H), 7.91 (d, J=8.6 Hz, 2H), 4.32-4.28 (m, 1H), 3.92 (s, 2H), 3.81 (s, 2H), 2.81 (d, J=4.4 Hz, 3H), 2.60-2.55 (m, 2H), 2.32-2.20 (m, 2H). m/z: [ESI⁺] 446 (M+H)⁺, (C₂₄H₂₃N₅O₂S).

### 2-(4-(Methylcarbamoyl)phenyl)-N-(piperidin-4-yl)benzo[d]imidazo[2,1-b]thiazole-7-carboxamide (Compound 185)

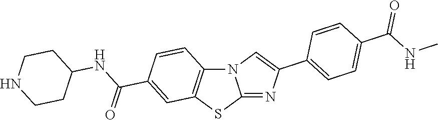 [SMILES: CNC(=O)c1ccc(-c2cn3c(n2)sc2cc(C(=O)NC4CCNCC4)ccc23)cc1]

Starting from tert-butyl 4-(2-(4-(methylcarbamoyl)phenyl)benzo[d]imidazo[2,1-b]thiazole-7-carboxamido)piperidine-1-carboxylate (120 mg, 0.225 mmol). Yield 24 mg (25%), as a white solid. ¹H NMR (400 MHz, DMSO) δ 8.94 (s, 1H), 8.51 (s, 1H), 8.46 (q, J=4.4 Hz, 1H), 8.41 (d, J=7.6 Hz, 1H), 8.05 (s, 2H), 7.95 (d, J=8.6 Hz, 2H), 7.91 (d, J=8.6 Hz, 2H), 3.92-3.80 (m, 1H), 2.99 (qd, J=3.8, 11.2 Hz, 2H), 2.81 (d, J=4.4 Hz, 3H), 2.60-2.56 (m, 2H), 1.84-1.74 (m, 2H), 1.46 (dq, J=4.0, 12.0 Hz, 2H). m/z: [ESI⁺] 434 (M+H)⁺, (C₂₃H₂₃N₅O₂S).

### (S)-2-(4-(methylcarbamoyl)phenyl)-N-(piperidin-3-yl)benzo[d]imidazo[2,1-b]thiazole-7-carboxamide formate (Compound 227)

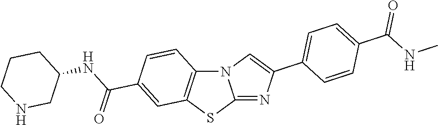 [SMILES: CNC(=O)c1ccc(-c2cn3c(n2)sc2cc(C(=O)N[C@H]4CCCNC4)ccc23)cc1]

Starting from tert-butyl (S)-3-(2-(4-(methylcarbamoyl)phenyl)benzo[d]imidazo[2,1-b]thiazole-7-carboxamido)piperidine-1-carboxylate (150 mg, 0.281 mmol). Yield 60 mg (45%), as a white solid. ¹H NMR (400 MHz, DMSO) δ 8.93 (s, 1H), 8.54 (s, 2H), 8.45 (q, J=4.4 Hz, 1H), 8.33 (s, 1H), 8.07 (s, 2H), 7.95 (d, J=8.6 Hz, 2H), 7.91 (d, J=8.6 Hz, 2H), 4.09-4.01 (m, 1H), 3.17 (dd, J=4.0, 12.0 Hz, 1H), 3.03-2.96 (m, 1H), 2.81 (d, J=4.4 Hz, 3H), 2.68-2.60 (m, 2H), 1.92-1.90 (m, 1H), 1.84-1.77 (m, 1H), 1.59 (t, J=9.2 Hz, 2H). m/z: [ESI⁺] 434 (M+H)⁺, (C₂₃H₂₃N₅O₂S).

### N-(4-(methylamino)butyl)-2-(4-(methylcarbamoyl)phenyl)benzo[d]imidazo[2,1-b]thiazole-7-carboxamide formate (Compound 229)

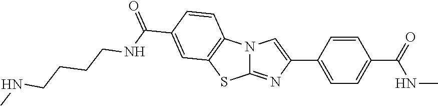

Starting from tert-butyl methyl(4-(2-(4-(methylcarbamoyl)phenyl)benzo[d]imidazo[2,1-b]thiazole-7-carboxamido)butyl)carbamate (220 mg, 0.411 mmol). Yield 16 mg (9%), as a white solid. ¹H NMR (400 MHz, DMSO) δ 8.93 (s, 1H), 8.70 (s, 1H), 8.51 (s, 1H), 8.46 (q, J=4.4 Hz, 1H), 8.40 (s, 1H), 8.06 (s, 2H), 7.95 (d, J=8.6 Hz, 2H), 7.91 (d, J=8.6 Hz, 2H), 3.33-3.31 (m, 2H), 2.81 (d, J=4.4 Hz, 3H), 2.75 (s, 2H), 2.43 (s, 3H), 1.59 (s, 4H). m/z: [ESI⁺] 436 (M+H)⁺, (C₂₃H₂₅N₅O₂S).

### N-((1-oxa-8-azaspiro[4.5]decan-2-yl)methyl)-2-(4-(methylcarbamoyl)phenyl)benzo[d]imidazo[2,1-b]thiazole-7-carboxamide formate (Compound 230)

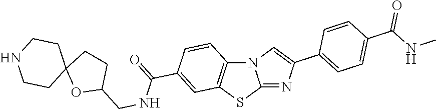 [SMILES: CNC(=O)c1ccc(-c2cn3c(n2)sc2cc(C(=O)NCC4CCC5(CCNCC5)O4)ccc23)cc1]

Starting from tert-butyl 2-((2-(4-(methylcarbamoyl)phenyl)benzo[d]imidazo[2,1-b]thiazole-7-carboxamido)methyl)-1-oxa-8-azaspiro[4.5]decane-8-carboxylate (160 mg, 0.265 mmol). Yield 44 mg (30%), as a white solid. ¹H NMR (400 MHz, DMSO) δ 8.94 (s, 1H), 8.68 (d, J=6.0 Hz, 1H), 8.52 (d, J=1.2 Hz, 1H), 8.47 (q, J=4.4 Hz, 1H), 8.40 (s, 1H), 8.09-8.03 (m, 2H), 7.95 (d, J=8.6 Hz, 2H), 7.91 (d, J=8.6 Hz, 2H), 4.13-4.11 (m, 1H), 3.46-3.30 (m, 2H), 3.06-2.87 (m, 4H), 2.81 (d, J=4.4 Hz, 3H), 2.07-1.97 (m, 1H), 1.81-1.72 (m, 3H), 1.65 (d, J=10.6 Hz, 4H). m/z: [ESI⁺] 504 (M+H)⁺, (C₂₇H₂₉N₅O₃S).

### N-((2-azaspiro[3.3]heptan-6-yl)methyl)-2-(4-(methylcarbamoyl)phenyl)benzo[d]imidazo[2,1-b]thiazole-7-carboxamide (Compound 272)

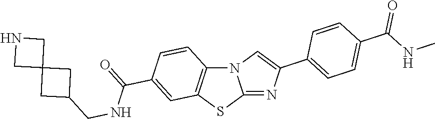 [SMILES: CNC(=O)c1ccc(-c2cn3c(n2)sc2cc(C(=O)NCC4CC5(CNC5)C4)ccc23)cc1]

Starting from tert-butyl 6-((2-(4-(methylcarbamoyl)phenyl)benzo[d]imidazo[2,1-b]thiazole-7-carboxamido)methyl)-2-azaspiro[3.3]heptane-2-carboxylate (150 mg, 0.268 mmol). Yield 40 mg (32%), as a white solid. ¹H NMR (400 MHz, DMSO) δ 8.80 (s, 1H), 8.44 (d, J=1.2 Hz, 1H), 8.27 (t, J=6.0 Hz, 1H), 8.14 (d, J=4.8 Hz, 1H), 8.03 (d, J=1.2 Hz, 2H), 7.95 (d, J=8.6 Hz, 2H), 7.91 (d, J=8.6 Hz, 2H), 3.53 (s, 2H), 3.48 (s, 2H), 3.32 (dd, J=5.6, 7.2 Hz, 2H), 2.84 (d, J=4.4 Hz, 3H), 2.40 (p, J=7.4 Hz, 1H), 2.27-2.17 (m, 2H), 1.95-1.84 (m, 2H). m/z: [ESI⁺] 460 (M+H)⁺, (C₂₅H₂₅N₅O₂S).

### N-((1s,3s)-3-(methylamino)cyclobutyl)-2-(4-(methylcarbamoyl)phenyl)benzo[d]imidazo[2,1-b]thiazole-7-carboxamide formate (Compound 246)

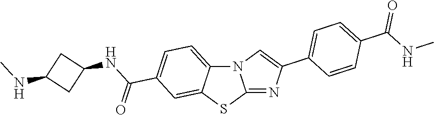

Starting from tert-butyl methyl((1s,3s)-3-(2-(4-(methylcarbamoyl) phenyl)benzo[d]imidazo[2,1-b]thiazole-7-carboxamido)cyclobutyl)carbamate (100 mg, 0.187 mmol). Yield 16 mg (18%), as an off-white solid. ¹H NMR (400 MHz, DMSO) δ 8.98 (d, J=7.2 Hz, 1H), 8.92 (s, 1H), 8.53 (d, J=1.6 Hz, 1H), 8.46 (q, J=4.4 Hz, 1H), 8.09 (d, J=8.6 Hz, 1H), 8.05 (d, J=8.6 Hz, 1H), 7.95 (d, J=8.6 Hz, 2H), 7.91 (d, J=8.6 Hz, 2H), 4.28-4.15 (m, 1H), 3.16-3.14 (m, 1H), 2.81 (d, J=4.4 Hz, 3H), 2.62-2.58 (m, 2H), 2.34 (s, 3H), 2.09 (dq, J=3.2, 10.6 Hz, 2H). m/z: [ESI⁺] 434 (M+H)⁺, (C₂₃H₂₃N₅O₂S).

### N-((1r,3r)-3-(methylamino)cyclobutyl)-2-(4-(methylcarbamoyl)phenyl)benzo[d]imidazo[2,1-b]thiazole-7-carboxamide (Compound 247)

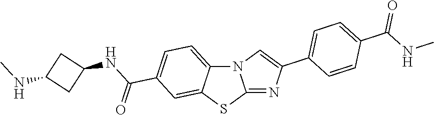

Starting from tert-butyl methyl((1r,3r)-3-(2-(4-(methylcarbamoyl) phenyl)benzo[d]imidazo[2,1-b]thiazole-7-carboxamido)cyclobutyl)carbamate (200 mg, 0.375 mmol). Yield 99 mg (61%), as an off-white solid. ¹H NMR (400 MHz, DMSO) δ 8.93 (s, 1H), 8.75 (d, J=7.2 Hz, 1H), 8.50 (s, 1H), 8.45 (q, J=4.4 Hz, 1H), 8.06 (s, 2H), 7.95 (d, J=8.6 Hz, 2H), 7.91 (d, J=8.6 Hz, 2H), 4.57-4.42 (m, 1H), 3.23-3.20 (m, 1H), 2.81 (d, J=4.4 Hz, 3H), 2.24-2.16 (m, 2H), 2.21 (s, 3H), 2.09 (dt, J=4.0, 8.2 Hz, 2H). m/z: [ESI⁺] 434 (M+H)⁺, (C₂₃H₂₃N₅O₂S).

### N-(3-aminocyclohexyl)-2-(4-(methylcarbamoyl)phenyl)benzo[d]imidazo[2,1-b]thiazole-7-carboxamide formate (Compound 270)

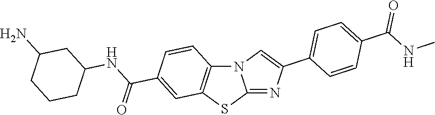 [SMILES: CNC(=O)c1ccc(-c2cn3c(n2)sc2cc(C(=O)NC4CCCC(N)C4)ccc23)cc1]

Starting from tert-butyl (3-(2-(4-(methylcarbamoyl)phenyl)benzo[d]imidazo[2,1-b]thiazole-7-carboxamido)cyclohexyl)carbamate (150 mg, 0.274 mmol). Yield 12 mg (9%), as an off-white solid. ¹H NMR (400 MHz, DMSO) δ 8.95 (s Hz, 1H), 8.52 (s, 1H), 8.46 (q, J=4.4 Hz, 1H), 8.37 (s, 1H), 8.29 (d, J=7.2 Hz, 1H), 8.06 (s, 2H), 7.95 (d, J=8.6 Hz, 2H), 7.91 (d, J=8.6 Hz, 2H), 4.30 (s, 0.8H), 3.90 (s, 0.2H), 3.30-3.26 (m, 0.8H), 3.10-3.00 (m, 0.2H), 2.81 (d, J=4.4 Hz, 3H), 1.93-1.80 (m, 1H), 1.75-1.62 (m, 4H), 1.55 (s, 1H), 1.45 (s, 1H), 1.38-1.21 (m, 1H). (A mixture of cis/trans isomers with a ratio of 1:4). m/z: [ESI⁺] 448 (M+H)⁺, (C₂₄H₂₅N₅O₂S).

### (S)—N-(pyrrolidin-3-ylmethyl)-2-(p-tolyl)benzo[d]imidazo[2,1-b]thiazole-7-carboxamide formate (Compound 114)

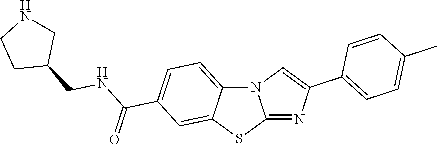 [SMILES: Cc1ccc(-c2cn3c(n2)sc2cc(C(=O)NC[C@H]4CCNC4)ccc23)cc1]

Starting from tert-butyl 3-((2-(p-tolyl)benzo[d]imidazo[2,1-b]thiazole-7-carboxamido)methyl)pyrrolidine-1-carboxylate (400 mg, 0.203 mmol). Yield 14 mg (4%), as an off-white solid. ¹H NMR (400 MHz, DMSO) δ 8.85 (s, 1H), 8.76 (s, 1H), 8.51 (s, 1H), 8.40 (s, 1H), 8.04 (s, 2H), 7.77 (d, J=8.0 Hz, 2H), 7.26 (d, J=8.0 Hz, 2H), 3.44-3.29 (m, 3H), 3.22-3.13 (m, 2H), 3.09-2.98 (m, 1H), 2.89 (s, 1H), 2.34 (s, 3H), 1.97 (dq, J=7.6, 13.6 Hz, 1H), 1.63 (dq, J=7.6, 14.4 Hz, 1H). m/z: [ESI⁺] 391 (M+H)⁺, (C₂₂H₂₂N₄OS).

### (R)—N-(pyrrolidin-3-ylmethyl)-2-(p-tolyl)benzo[d]imidazo[2,1-b]thiazole-7-carboxamideformate (Compound 116)

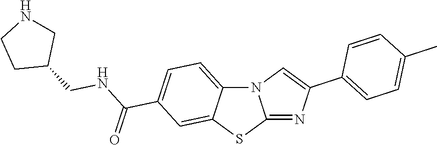 [SMILES: Cc1ccc(-c2cn3c(n2)sc2cc(C(=O)NC[C@@H]4CCNC4)ccc23)cc1]

Starting from tert-butyl 3-((2-(p-tolyl)benzo[d]imidazo[2,1-b]thiazole-7-carboxamido)methyl)pyrrolidine-1-carboxylate (400 mg, 0.815 mmol). Yield 23 mg (7%), as an off-white solid. ¹H NMR (400 MHz, DMSO) δ 8.85 (s, 1H), 8.76 (s, 1H), 8.51 (s, 1H), 8.40 (s, 1H), 8.04 (s, 2H), 7.77 (d, J=8.0 Hz, 2H), 7.26 (d, J=8.0 Hz, 2H), 3.44-3.29 (m, 3H), 3.22-3.13 (m, 2H), 3.09-2.98 (m, 1H), 2.89 (s, 1H), 2.34 (s, 3H), 1.97 (dq, J=7.6, 13.6 Hz, 1H), 1.63 (dq, J=7.6, 14.4 Hz, 1H). m/z: [ESI⁺] 391 (M+H)⁺, (C₂₂H₂₂N₄OS).

### 2-(4-(aminomethyl)-2-cyclopropylphenyl)-N-(3-(piperidin-1-yl)propyl)benzo[d]imidazo[2,1-b]thiazole-7-carboxamide diformate (Compound 370)

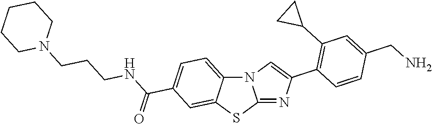 [SMILES: NCc1ccc(-c2cn3c(n2)sc2cc(C(=O)NCCCN4CCCCC4)ccc23)c(C2CC2)c1]

Compound 2-(4-(aminomethyl)-2-cyclopropylphenyl)-N-(3-(piperidin-1-yl)propyl)benzo[d]imidazo[2,1-b]thiazole-7-carboxamide diformate was prepared from tert-butyl (3-cyclopropyl-4-(7-((3-(piperidin-1-yl)propyl)carbamoyl)benzo[d]imidazo[2,1-b]thiazol-2-yl)benzyl)carbamate (100 mg, 0.170 mmol) following a similar procedure to that described for the synthesis of 4-amino-1-(4-bromo-3-fluorophenyl)butan-1-one hydrochloride and was isolated as a light yellow solid.

Yield 23 mg (24%). ¹H NMR (400 MHz, DMSO) δ 8.69-8.65 (m, 2H), 8.50 (d, J=1.6 Hz, 1H), 8.42-8.29 (m, 1.74H, formic acid), 8.23 (d, J=8.4 Hz, 1H), 8.08-7.99 (m, 1H), 7.93 (dd, J=2.0, 8.0 Hz, 1H), 7.36-7.27 (m, 1H), 7.25-7.17 (m, 1H), 3.98-3.94 (m, 2H), 3.38-3.31 (m, 2H), 2.55-2.53 (m, 7H), 1.80-1.66 (m, 2H), 1.55-1.47 (m, 4H), 1.45-1.35 (m, 2H), 1.17-1.07 (m, 2H), 0.84-0.72 (m, 2H). NH₂ protons not observed. m/z: [ESI⁺] 488 (M+H)⁺. (C₂₈H₃₃N₅OS).

### 2-(2-fluoro-4-(pyrrolidin-2-yl)phenyl)-N-(3-(piperidin-1-yl)propyl)benzo[d]imidazo[2,1-b]thiazole-7-carboxamide diformate (Compound 416)

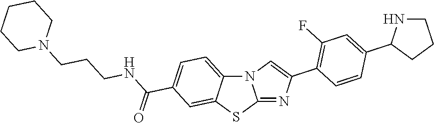 [SMILES: O=C(NCCCN1CCCCC1)c1ccc2c(c1)sc1nc(-c3ccc(C4CCCN4)cc3F)cn12]

Compound 2-(2-fluoro-4-(pyrrolidin-2-yl)phenyl)-N-(3-(piperidin-1-yl)propyl)benzo[d]imidazo[2,1-b]thiazole-7-carboxamide diformate was prepared from tert-butyl 2-(3-fluoro-4-(7-((3-(piperidin-1-yl)propyl)carbamoyl)benzo[d]imidazo[2,1-b]thiazol-2-yl)phenyl)pyrrolidine-1-carboxylate (300 mg, 0.495 mmol) following a similar procedure to that described for the synthesis of 4-amino-1-(4-bromo-3-fluorophenyl)butan-1-one hydrochloride and was isolated as a yellow solid.

Yield 32 mg (11%). ¹H NMR (400 MHz, DMSO) δ 8.75-8.67 (m, 2H), 8.50 (d, J=1.6 Hz, 1H), 8.36-8.29 (m, 2H, formic acid), 8.27 (dd, J=2.4, 8.4 Hz, 1H), 8.16-8.12 (m, 1H), 8.02 (dd, J=1.6, 8.4 Hz, 1H), 7.45 (dd, J=1.6, 12.4 Hz, 1H), 7.37 (d, J=8.0 Hz, 1H), 4.51-4.32 (m, 1H), 3.39-3.30 (m, 2H), 3.28-3.10 (m, 2H), 2.65-2.53 (m, 6H), 2.35-2.26 (m, 1H), 2.06-1.72 (m, 5H), 1.64-1.51 (m, 4H), 1.47-1.36 (m, 2H). Aliphatic NH proton not observed. ¹⁹F NMR (376 MHz, DMSO) δ −112.83. m/z: [ESI⁺] 506 (M+H)⁺. (C₂₈H₃₂FN₅OS).

### (R)-2-(2-fluoro-4-(pyrrolidin-2-yl)phenyl)-N-(3-(piperidin-1-yl)propyl)benzo[d]imidazo[2,1-b]thiazole-7-carboxamide dihydrochloride (Compound 417)

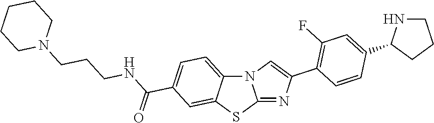 [SMILES: O=C(NCCCN1CCCCC1)c1ccc2c(c1)sc1nc(-c3ccc([C@H]4CCCN4)cc3F)cn12]

Compound (R)-2-(2-fluoro-4-(pyrrolidin-2-yl)phenyl)-N-(3-(piperidin-1-yl)propyl)benzo[d]imidazo[2,1-b]thiazole-7-carboxamide dihydrochloride was prepared from tert-butyl (R)-2-(3-fluoro-4-(7-((3-(piperidin-1-yl)propyl)carbamoyl)benzo[d]imidazo[2,1-b]thiazol-2-yl)phenyl)pyrrolidine-1-carboxylate (130 mg, 0.215 mmol) following a similar procedure to that described for the synthesis of 4-amino-1-(4-bromo-3-fluorophenyl)butan-1-one hydrochloride and was isolated as an off-white solid.

Yield 64 mg (52%). ¹H NMR (400 MHz, DMSO) δ 10.26 (br s, 2H, NH₂+), 9.18 (br s, 1H, NH⁺), 8.92 (t, J=5.6 Hz, 1H), 8.80 (d, J=3.6 Hz, 1H), 8.58 (d, J=1.6 Hz, 1H), 8.31 (d, J=8.4 Hz, 1H), 8.26-8.15 (m, 1H), 8.10 (dd, J=1.6, 8.4 Hz, 1H), 7.63 (dd, J=1.6, 12.4 Hz, 1H), 7.48 (dd, J=1.6, 8.4 Hz, 1H), 4.66-4.55 (m, 1H), 3.47-3.34 (m, 5H), 3.34-3.24 (m, 1H), 3.14-3.03 (m, 2H), 2.91-2.78 (m, 2H), 2.48-2.36 (m, 1H), 2.19-1.92 (m, 5H), 1.84-1.75 (m, 4H), 1.74-1.65 (m, 1H), 1.45-1.30 (m, 1H). ¹⁹F NMR (376 MHz, DMSO) δ −112.75. m/z: [ESI⁺] 506 (M+H)⁺. (C₂₈H₃₂FN₅OS).

### (S)-2-(2-fluoro-4-(pyrrolidin-2-yl)phenyl)-N-(3-(piperidin-1-yl)propyl)benzo[d]imidazo[2,1-b]thiazole-7-carboxamide dihydrochloride (Compound 418)

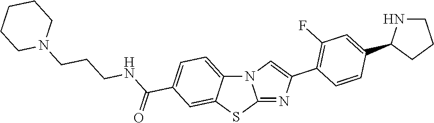 [SMILES: O=C(NCCCN1CCCCC1)c1ccc2c(c1)sc1nc(-c3ccc([C@@H]4CCCN4)cc3F)cn12]

Compound (S)-2-(2-fluoro-4-(pyrrolidin-2-yl)phenyl)-N-(3-(piperidin-1-yl)propyl)benzo[d]imidazo[2,1-b]thiazole-7-carboxamide dihydrochloride was prepared from tert-butyl (S)-2-(3-fluoro-4-(7-((3-(piperidin-1-yl)propyl)carbamoyl)benzo[d]imidazo[2,1-b]thiazol-2-yl)phenyl)pyrrolidine-1-carboxylate (130 mg, 0.215 mmol) following a similar procedure to that described for the synthesis of 4-amino-i-(4-bromo-3-fluorophenyl)butan-1-one hydrochloride, and was isolated as an off-white solid.

Yield 86 mg (69%). ¹H NMR (400 MHz, DMSO) δ 10.26 (br s, 2H, NH₂+), 9.18 (br s, 1H, NH⁺), 8.92 (t, J=5.6 Hz, 1H), 8.80 (d, J=3.6 Hz, 1H), 8.58 (d, J=1.6 Hz, 1H), 8.31 (d, J=8.4 Hz, 1H), 8.26-8.15 (m, 1H), 8.10 (dd, J=1.6, 8.4 Hz, 1H), 7.63 (dd, J=1.6, 12.4 Hz, 1H), 7.48 (dd, J=1.6, 8.4 Hz, 1H), 4.66-4.55 (m, 1H), 3.47-3.34 (m, 5H), 3.34-3.24 (m, 1H), 3.14-3.03 (m, 2H), 2.91-2.78 (m, 2H), 2.48-2.36 (m, 1H), 2.19-1.92 (m, 5H), 1.84-1.75 (m, 4H), 1.74-1.65 (m, 1H), 1.45-1.30 (m, 1H). ¹⁹F NMR (376 MHz, DMSO) δ −112.75. m/z: [ESI⁺] 506 (M+H)⁺. (C₂₈H₃₂FN₅OS).

### (R)-2-(2-fluoro-4-(pyrrolidin-2-yl)phenyl)-N-(3-(4-fluoropiperidin-1-yl)propyl)benzo[d]imidazo[2,1-b]thiazole-7-carboxamide dihydrochloride (Compound 419)

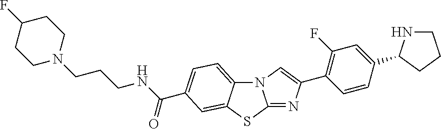 [SMILES: O=C(NCCCN1CCC(F)CC1)c1ccc2c(c1)sc1nc(-c3ccc([C@H]4CCCN4)cc3F)cn12]

Compound (R)-2-(2-fluoro-4-(pyrrolidin-2-yl)phenyl)-N-(3-(4-fluoropiperidin-1-yl)propyl)benzo[d]imidazo[2,1-b]thiazole-7-carboxamide dihydrochloride was prepared from tert-butyl (R)-2-(3-fluoro-4-(7-((3-(4-fluoropiperidin-1-yl)propyl)carbamoyl)benzo[d]imidazo[2,1-b]thiazol-2-yl)phenyl)pyrrolidine-1-carboxylate (200 mg, 0.321 mmol) following a similar procedure to that described for the synthesis of 4-amino-1-(4-bromo-3-fluorophenyl)butan-1-one hydrochloride and was isolated as a light yellow solid.

Yield 123 mg (64%). ¹H NMR (400 MHz, DMSO) δ 10.72 (br s, 1H, NH⁺), 10.20 (br s, 1H, NH⁺), 9.14 (br s, 1H, NH⁺), 8.88 (t, J=5.6 Hz, 1H), 8.80 (d, J=3.6 Hz, 1H), 8.57 (d, J=1.6 Hz, 1H), 8.31 (d, J=8.4 Hz, 1H), 8.27-8.15 (m, 1H), 8.09 (dd, J=1.6, 8.4 Hz, 1H), 7.62 (dd, J=1.6, 12.4 Hz, 1H), 7.48 (dd, J=1.6, 8.4 Hz, 1H), 5.01 (d, J=48.0 Hz, 0.70H), 4.93-4.70 (m, 0.30H), 4.67-4.55 (m, 1H), 3.58-3.48 (m, 1H), 3.48-3.35 (m, 4H), 3.35-3.22 (m, 1H), 3.21-2.96 (m, 4H), 2.47-2.36 (m, 1H), 2.30-1.95 (m, 9H). ¹⁹F NMR (376 MHz, DMSO) δ −112.76, −175.58 (0.30F), −186.62 (0.70F). m/z: [ESI⁺] 524 (M+H)⁺. (C₂₈H₃₁F₂N₅OS).

### (S)-2-(2-fluoro-4-(pyrrolidin-2-yl)phenyl)-N-(3-(4-fluoropiperidin-1-yl)propyl)benzo[d]imidazo[2,1-b]thiazole-7-carboxamide dihydrochloride (Compound 420)

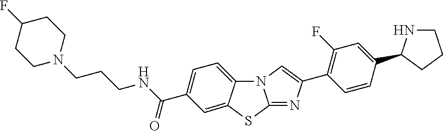 [SMILES: O=C(NCCCN1CCC(F)CC1)c1ccc2c(c1)sc1nc(-c3ccc([C@@H]4CCCN4)cc3F)cn12]

Compound (S)-2-(2-fluoro-4-(pyrrolidin-2-yl)phenyl)-N-(3-(4-fluoropiperidin-1-yl)propyl)benzo[d]imidazo[2,1-b]thiazole-7-carboxamide dihydrochloride was prepared from tert-butyl (S)-2-(3-fluoro-4-(7-((3-(4-fluoropiperidin-1-yl)propyl)carbamoyl)benzo[d]imidazo[2,1-b]thiazol-2-yl)phenyl)pyrrolidine-1-carboxylate (200 mg, 0.321 mmol) following a similar procedure to that described for the synthesis of 4-amino-1-(4-bromo-3-fluorophenyl)butan-1-one hydrochloride and was isolated as a light yellow solid.

Yield 116 mg (61%). ¹H NMR (400 MHz, DMSO) δ 10.72 (br s, 1H, NH⁺), 10.20 (br s, 1H, NH⁺), 9.14 (br s, 1H, NH⁺), 8.88 (t, J=5.6 Hz, 1H), 8.80 (d, J=3.6 Hz, 1H), 8.57 (d, J=1.6 Hz, 1H), 8.31 (d, J=8.4 Hz, 1H), 8.27-8.15 (m, 1H), 8.09 (dd, J=1.6, 8.4 Hz, 1H), 7.62 (dd, J=1.6, 12.4 Hz, 1H), 7.48 (dd, J=1.6, 8.4 Hz, 1H), 5.01 (d, J=48.0 Hz, 0.70H), 4.93-4.70 (m, 0.30H), 4.67-4.55 (m, 1H), 3.58-3.48 (m, 1H), 3.48-3.35 (m, 4H), 3.35-3.22 (m, 1H), 3.21-2.96 (m, 4H), 2.47-2.36 (m, 1H), 2.30-1.95 (m, 9H). ¹⁹F NMR (376 MHz, DMSO) δ −112.76, −175.58 (0.30F), −186.62 (0.70F). m/z: [ESI⁺] 524 (M+H)⁺. (C₂₈H₃₁F₂N₅OS).

### (R)—N-(3-(diethylamino)propyl)-2-(4-(pyrrolidin-2-yl)phenyl)benzo[d]imidazo[2,1-b]thiazole-7-carboxamide dihydrochloride (Compound 414)

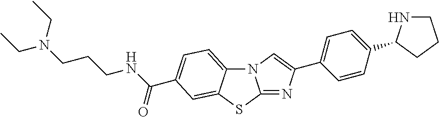 [SMILES: CCN(CC)CCCNC(=O)c1ccc2c(c1)sc1nc(-c3ccc([C@H]4CCCN4)cc3)cn12]

Compound (R)—N-(3-(diethylamino)propyl)-2-(4-(pyrrolidin-2-yl)phenyl)benzo[d]imidazo[2,1-b]thiazole-7-carboxamide dihydrochloride was prepared from tert-butyl (R)-2-(4-(7-((3-(diethylamino)propyl)carbamoyl)benzo[d]imidazo[2,1-b]thiazol-2-yl)phenyl)pyrrolidine-1-carboxylate (200 mg, 0.347 mmol) following a similar procedure to that described for the synthesis of 4-amino-i-(4-bromo-3-fluorophenyl)butan-1-one hydrochloride and was isolated as a white solid.

Yield 79 mg (41%). ¹H NMR (400 MHz, DMSO) δ 9.94 (br s, 2H, NH₂+), 8.91 (s, 1H), 8.99-8.82 (m, 2H), 8.56 (s, 1H), 8.14-8.06 (m, 2H), 7.95 (d, J=8.0 Hz, 2H), 7.61 (d, J=8.0 Hz, 2H), 4.63-4.52 (m, 1H), 3.46-3.26 (m, 4H), 3.19-3.05 (m, 6H), 2.45-2.37 (m, 1H), 2.20-2.00 (m, 3H), 1.99-1.88 (m, 2H), 1.22 (t, J=7.2 Hz, 6H). m/z: [ESI⁺] 476 (M+H)⁺. (C₂₇H₃₃N₅OS).

### (S)—N-(3-(diethylamino)propyl)-2-(4-(pyrrolidin-2-yl)phenyl)benzo[d]imidazo[2,1-b]thiazole-7-carboxamide dihydrochloride (Compound 415)

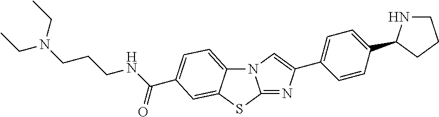 [SMILES: CCN(CC)CCCNC(=O)c1ccc2c(c1)sc1nc(-c3ccc([C@@H]4CCCN4)cc3)cn12]

Compound (S)—N-(3-(diethylamino)propyl)-2-(4-(pyrrolidin-2-yl)phenyl)benzo[d]imidazo[2,1-b]thiazole-7-carboxamide dihydrochloride was prepared from tert-butyl (S)-2-(4-(7-((3-(diethylamino)propyl)carbamoyl)benzo[d]imidazo[2,1-b]thiazol-2-yl)phenyl)pyrrolidine-1-carboxylate (200 mg, 0.347 mmol) following a similar procedure to that described for the synthesis of 4-amino-i-(4-bromo-3-fluorophenyl)butan-1-one hydrochloride, and was isolated as an off-white solid.

Yield 113 mg (59%). ¹H NMR (400 MHz, DMSO) δ 10.37-9.98 (m, 2H, NH₂+), 9.08-8.85 (m, 3H), 8.58 (s, 1H), 8.14-8.06 (m, 2H), 7.94 (d, J=8.0 Hz, 2H), 7.62 (d, J=8.0 Hz, 2H), 4.63-4.52 (m, 1H), 3.48-3.25 (m, 4H), 3.20-3.01 (m, 6H), 2.46-2.36 (m, 1H), 2.20-1.89 (m, 5H), 1.22 (t, J=7.2 Hz, 6H). m/z: [ESI⁺]476 (M+H)⁺. (C₂₇H₃₃N₅OS).

### 2-(4-((cyclopropylamino)methyl)-2-fluorophenyl)-N-(3-(diethylamino)propyl)benzo[d]imidazo[2,1-b]thiazole-7-carboxamide (Compound 331)

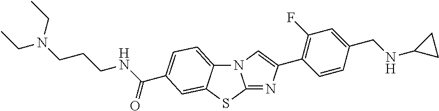 [SMILES: CCN(CC)CCCNC(=O)c1ccc2c(c1)sc1nc(-c3ccc(CNC4CC4)cc3F)cn12]

Compound 2-(4-((cyclopropylamino)methyl)-2-fluorophenyl)-N-(3-(diethylamino)propyl)benzo[d]imidazo[2,1-b]thiazole-7-carboxamide was prepared from tert-butyl cyclopropyl(4-(7-((3-(diethylamino)propyl)carbamoyl)benzo[d]imidazo[2,1-b]thiazol-2-yl)-3-fluorobenzyl)carbamate (2.40 g, 4.04 mmol,) following a similar procedure to that described for the synthesis of 4-amino-i-(4-bromo-3-fluorophenyl)butan-1-one hydrochloride, and was isolated as an off-white solid.

Yield 779 mg (39%). ¹H NMR (400 MHz, DMSO) δ 8.67 (d, J=3.6 Hz, 1H), 8.61 (t, J=5.6 Hz, 1H), 8.48 (d, J=1.6 Hz, 1H), 8.25 (d, J=8.4 Hz, 1H), 8.10-8.04 (m, 1H), 8.00 (dd, J=1.6, 8.4 Hz, 1H), 7.32-7.21 (m, 2H), 3.76 (s, 2H), 3.38-3.33 (m, 2H), 2.85 (br s, 1H), 2.49-2.42 (m, 6H), 2.10-2.01 (m, 1H), 1.74-1.60 (m, 2H), 0.96 (t, J=7.2 Hz, 6H), 0.40-0.33 (m, 2H), 0.30-0.22 (m, 2H). ¹⁹F NMR (376 MHz, DMSO) δ −114.23. m/z: [ESI⁺] 494 (M+H)⁺. (C₂₇H₃₂FN₅OS).

### 2-(4-(aminomethyl)-3-chloro-5-fluorophenyl)-N-(3-(piperidin-1-yl)propyl)benzo[d]imidazo[2,1-b]thiazole-7-carboxamide hemi-formate (Compound 384)

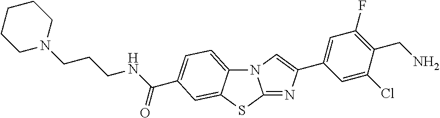 [SMILES: NCc1c(F)cc(-c2cn3c(n2)sc2cc(C(=O)NCCCN4CCCCC4)ccc23)cc1Cl]

Compound 2-(4-(aminomethyl)-3-chloro-5-fluorophenyl)-N-(3-(piperidin-1-yl)propyl)benzo[d]imidazo[2,1-b]thiazole-7-carboxamide hemi-formate was prepared from tert-butyl (2-chloro-6-fluoro-4-(7-((3-(piperidin-1-yl)propyl)carbamoyl)benzo[d]imidazo[2,1-b]thiazol-2-yl)benzyl)carbamate (200 mg, 0.333 mmol) following a similar procedure to that described for the synthesis of 4-amino-i-(4-bromo-3-fluorophenyl)butan-1-one hydrochloride, and was isolated as an off-white solid

Yield 24 mg (12%). ¹H NMR (400 MHz, DMSO) δ 9.00 (s, 1H), 8.75-8.65 (m, 1H), 8.51 (d, J=1.6 Hz, 1H), 8.31-8.22 (m, 1.98H, formic acid), 8.07-7.95 (m, 2H), 7.81 (s, 1H), 7.69-7.63 (m, 1H), 3.99-3.85 (m, 2H), 3.38-3.26 (m, 2H), 2.48-2.36 (m, 6H), 1.81-1.66 (m, 2H), 1.61-1.47 (m, 4H), 1.45-1.34 (m, 2H). NH₂ protons not observed. ¹⁹F NMR (376 MHz, DMSO) δ −113.53. m/z: [ESI⁺] 500, 502 (M+H)⁺. (C₂₅H₂₇ClFN₅OS).

### 2-(4-(aminomethyl)-3,5-difluorophenyl)-N-(3-(piperidin-1-yl)propyl)benzo[d]imidazo[2,1-b]thiazole-7-carboxamide hemi-formate (Compound 383)

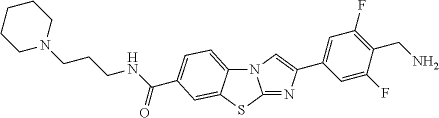 [SMILES: NCc1c(F)cc(-c2cn3c(n2)sc2cc(C(=O)NCCCN4CCCCC4)ccc23)cc1F]

Compound 2-(4-(aminomethyl)-3,5-difluorophenyl)-N-(3-(piperidin-1-yl)propyl)benzo[d]imidazo[2,1-b]thiazole-7-carboxamide hemi-formate was prepared from tert-butyl (2,6-difluoro-4-(7-((3-(piperidin-1-yl)propyl)carbamoyl)benzo[d]imidazo[2,1-b]thiazol-2-yl)benzyl)carbamate (300 mg, 0.514 mmol) following a similar procedure to that described for the synthesis of 4-amino-i-(4-bromo-3-fluorophenyl)butan-1-one hydrochloride, and was isolated as an off-white solid

Yield 35 mg (12%). ¹H NMR (400 MHz, DMSO) δ 8.96 (s, 1H), 8.68 (t, J=5.6 Hz, 1H), 8.51 (d, J=1.6 Hz, 1H), 8.26 (s, 2.17H, formic acid), 8.04 (dd, J=1.6, 8.4 Hz, 1H), 7.98 (d, J=8.4 Hz, 1H), 7.56 (m, 2H), 3.88 (s, 2H), 3.39-3.27 (m, 2H), 2.50-2.32 (m, 6H), 1.83-1.69 (m, 2H), 1.63-1.49 (m, 4H), 1.46-1.32 (m, 2H). NH₂ protons not observed. ¹⁹F NMR (376 MHz, DMSO) δ −115.07. m/z: [ESI⁺] 484 (M+H)⁺. (C₂₅H₂₇F₂N₅OS).

### 2-(4-(aminomethyl)-3-methylphenyl)-N-(3-(piperidin-1-yl)propyl)benzo[d]imidazo[2,1-b]thiazole-7-carboxamide hemi-formate (Compound 381)

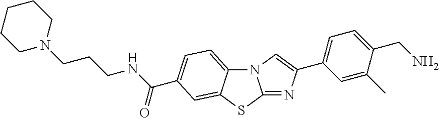 [SMILES: Cc1cc(-c2cn3c(n2)sc2cc(C(=O)NCCCN4CCCCC4)ccc23)ccc1CN]

Compound 2-(4-(aminomethyl)-3-methylphenyl)-N-(3-(piperidin-1-yl)propyl)benzo[d]imidazo[2,1-b]thiazole-7-carboxamide hemi-formate was prepared from tert-butyl (2-methyl-4-(7-((3-(piperidin-1-yl)propyl)carbamoyl)benzo[d]imidazo[2,1-b]thiazol-2-yl)benzyl)carbamate (251 mg, 0.447 mmol) following a similar procedure to that described for the synthesis of 4-amino-i-(4-bromo-3-fluorophenyl)butan-1-one hydrochloride, and was isolated as a brown solid

Yield 82 mg (33%). ¹H NMR (400 MHz, DMSO) δ 8.82 (s, 1H), 8.67 (t, J=5.6 Hz, 1H), 8.49 (s, 1H), 8.32 (s, 2.18H, formic acid), 8.04 (s, 2H), 7.76-7.70 (m, 2H), 7.45 (d, J=8.0 Hz, 1H), 3.98 (s, 2H), 3.38-3.28 (m, 2H), 2.48-2.36 (m, 9H), 1.78-1.69 (m, 2H), 1.58-1.48 (m, 4H), 1.45-1.35 (m, 2H). NH₂ protons not observed. m/z: [ESI⁺] 462 (M+H)⁺. (C₂₆H₃₁N₅OS).

### 2-(4-(aminomethyl)-3-(difluoromethyl)phenyl)-N-(3-(piperidin-1-yl)propyl)benzo[d]imidazo[2,1-b]thiazole-7-carboxamide hemi-formate (Compound 379)

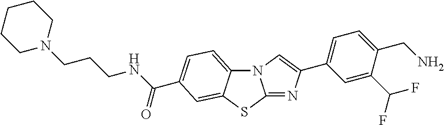 [SMILES: NCc1ccc(-c2cn3c(n2)sc2cc(C(=O)NCCCN4CCCCC4)ccc23)cc1C(F)F]

Compound 2-(4-(aminomethyl)-3-(difluoromethyl)phenyl)-N-(3-(piperidin-1-yl)propyl)benzo[d]imidazo[2,1-b]thiazole-7-carboxamide hemi-formate was prepared from tert-butyl (2-(difluoromethyl)-4-(7-((3-(piperidin-1-yl)propyl)carbamoyl)benzo[d]imidazo[2,1-b]thiazol-2-yl)benzyl)carbamate (100 mg, 0.167 mmol) following a similar procedure to that described for the synthesis of 4-amino-i-(4-bromo-3-fluorophenyl)butan-1-one hydrochloride, and was isolated as alight yellow solid.

Yield 20 mg (21%). ¹H NMR (400 MHz, DMSO) δ 8.95 (s, 1H), 8.69 (t, J=5.6 Hz, 1H), 8.50 (d, J=1.6 Hz, 1H), 8.29 (s, 1.42H, formic acid), 8.13-8.00 (m, 4H), 7.67 (d, J=8.0 Hz, 1H), 7.38 (t, J=54.8 Hz, 1H), 4.04 (s, 2H), 3.39-3.28 (m, 2H), 2.49-2.42 (m, 6H), 1.79-1.69 (m, 2H), 1.58-1.48 (m, 4H), 1.45-1.34 (m, 2H). NH₂ protons not observed. ¹⁹F NMR (376 MHz, DMSO) δ −111.50. m/z: [ESI⁺] 498 (M+H)⁺. (C₂₆H₂₉F₂N₅OS).

### 2-(4-(aminomethyl)-2-(difluoromethyl)phenyl)-N-(3-(piperidin-1-yl)propyl)benzo[d]imidazo[2,1-b]thiazole-7-carboxamide hemi-formate (Compound 371)

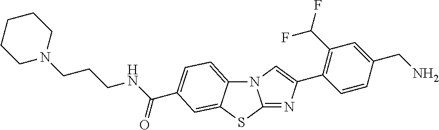 [SMILES: NCc1ccc(-c2cn3c(n2)sc2cc(C(=O)NCCCN4CCCCC4)ccc23)c(C(F)F)c1]

Compound 2-(4-(aminomethyl)-2-(difluoromethyl)phenyl)-N-(3-(piperidin-1-yl)propyl)benzo[d]imidazo[2,1-b]thiazole-7-carboxamide hemi-formate was prepared from tert-butyl (3-(difluoromethyl)-4-(7-((3-(piperidin-1-yl)propyl)carbamoyl)benzo[d]imidazo[2,1-b]thiazol-2-yl)benzyl)carbamate (200 mg, 0.335) following a similar procedure to that described for the synthesis of 4-amino-1-(4-bromo-3-fluorophenyl)butan-1-one hydrochloride, and was isolated as an off-white solid.

Yield 66 mg (33%). ¹H NMR (400 MHz, DMSO) δ 8.81-8.65 (m, 2H), 8.52 (s, 1H), 8.41-8.27 (m, 2.11H, formic acid), 8.16 (d, J=8.4 Hz, 1H), 8.05 (d, J=8.4 Hz, 1H), 7.92-7.82 (m, 2H), 7.75 (t, J=55.2 Hz, 1H), 7.72-7.65 (m, 1H), 4.12-4.01 (m, 2H), 3.39-3.29 (m, 2H), 2.49-2.42 (m, 6H), 1.85-1.69 (m, 2H), 1.63-1.48 (m, 4H), 1.45-1.27 (m, 2H). NH₂ protons not observed. ¹⁹F NMR (376 MHz, DMSO) δ −109.73. m/z: [ESI⁺] 498 (M+H)⁺. (C₂₆H₂₉F₂N₅OS).

### 2-(4-(aminomethyl)-3-chlorophenyl)-N-(3-(piperidin-1-yl)propyl)benzo[d]imidazo[2,1-b]thiazole-7-carboxamide hemi formate (Compound 376)

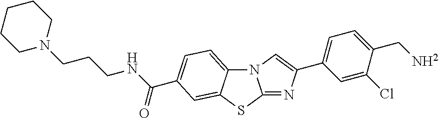 [SMILES: NCc1ccc(-c2cn3c(n2)sc2cc(C(=O)NCCCN4CCCCC4)ccc23)cc1Cl]

Compound 2-(4-(aminomethyl)-3-chlorophenyl)-N-(3-(piperidin-1-yl)propyl)benzo[d]imidazo[2,1-b]thiazole-7-carboxamide hemi-formate was prepared from tert-butyl (2-chloro-4-(7-((3-(piperidin-1-yl)propyl)carbamoyl)benzo[d]imidazo[2,1-b]thiazol-2-yl)benzyl)carbamate (139 mg, 0.239 mmol) following a similar procedure to that described for the synthesis of 4-amino-1-(4-bromo-3-fluorophenyl)butan-1-one hydrochloride, and was isolated as a white solid.

Yield 71 mg (55%). ¹H NMR (400 MHz, DMSO) δ 8.93 (s, 1H), 8.65 (t, J=5.6 Hz, 1H), 8.50 (d, J=1.6 Hz, 1H), 8.19 (s, 1.37H, formic acid), 8.06-8.00 (m, 2H), 7.93 (d, J=2.0 Hz, 1H), 7.86 (dd, J=1.6, 8.0 Hz, 1H), 7.64 (d, J=8.0 Hz, 1H), 3.95 (s, 2H), 3.34-3.32 (m, 2H), 2.45-2.36 (m, 6H), 1.78-1.68 (m, 2H), 1.57-1.48 (m, 4H), 1.44-1.35 (m, 2H). NH₂ protons not observed. m/z: [ESI⁺] 482, 484 (M+H)⁺. (C₂₅H₂₅ClN₅OS).

### 2-(4-(aminomethyl)-3-(trifluoromethyl)phenyl)-N-(3-(piperidin-1-yl)propyl)benzo[d]imidazo[2,1-b]thiazole-7-carboxamide hemi-formate (Compound 378)

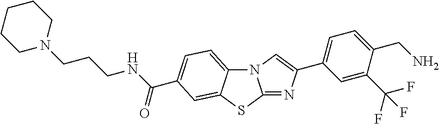 [SMILES: NCc1ccc(-c2cn3c(n2)sc2cc(C(=O)NCCCN4CCCCC4)ccc23)cc1C(F)(F)F]

Compound 2-(4-(aminomethyl)-3-(trifluoromethyl)phenyl)-N-(3-(piperidin-1-yl)propyl)benzo[d]imidazo[2,1-b]thiazole-7-carboxamide hemi-formate was prepared from tert-butyl (4-(7-((3-(piperidin-1-yl)propyl)carbamoyl)benzo[d]imidazo[2,1-b]thiazol-2-yl)-2-(trifluoromethyl)benzyl)carbamate (160 mg, 0.260 mmol) following a similar procedure to that described for the synthesis of 4-amino-i-(4-bromo-3-fluorophenyl)butan-1-one hydrochloride, and was isolated as a light yellow solid.

Yield 83 mg (47%). ¹H NMR (400 MHz, DMSO) δ 9.00 (s, 1H), 8.71 (d, J=5.6 Hz, 1H), 8.51 (s, 1H), 8.36 (s, 3.58H, formic acid), 8.19-8.12 (m, 2H), 8.06-8.01 (m, 2H), 7.88 (d, J=8.0 Hz, 1H), 3.98 (s, 2H), 3.37-3.28 (m, 2H), 2.47-2.35 (m, 6H), 1.78-1.68 (m, 2H), 1.56-1.48 (m, 4H), 1.44-1.36 (m, 2H). NH₂ protons not observed. ¹⁹F NMR (376 MHz, DMSO) δ −58.70. m/z: [ESI⁺] 516 (M+H)⁺. (C₂₆H₂₈F₃N₅OS).

### 2-(4-(aminomethyl)-2-chlorophenyl)-N-(3-(piperidin-1-yl)propyl)benzo[d]imidazo[2,1-b]thiazole-7-carboxamide dihydrochloride (Compound 369)

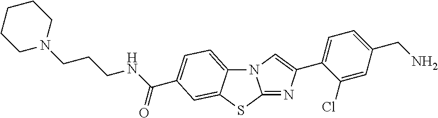 [SMILES: NCc1ccc(-c2cn3c(n2)sc2cc(C(=O)NCCCN4CCCCC4)ccc23)c(Cl)c1]

Compound 2-(4-(aminomethyl)-2-chlorophenyl)-N-(3-(piperidin-1-yl)propyl)benzo[d]imidazo[2,1-b]thiazole-7-carboxamide dihydrochloride was prepared from tert-butyl (3-chloro-4-(7-((3-(piperidin-1-yl)propyl)carbamoyl)benzo[d]imidazo[2,1-b]thiazol-2-yl)benzyl)carbamate (230 mg, 0.395 mmol) following a similar procedure to that described for the synthesis of 4-amino-1-(4-bromo-3-fluorophenyl)butan-1-one hydrochloride, and was isolated as a light yellow solid.

Yield 19 mg (9%). ¹H NMR (400 MHz, DMSO) δ 10.29 (br s, 1H, NH⁺), 9.04 (s, 1H), 8.92 (d, J=5.6 Hz, 1H), 8.58 (t, J=1.6 Hz, 1H), 8.53 (s, 3H, NH₃*), 8.32 (d, J=8.4 Hz, 1H), 8.23 (d, J=8.0 Hz, 1H), 8.11 (dd, J=1.6, 8.4 Hz, 1H), 7.77 (d, J=1.6 Hz, 1H), 7.57 (dd, J=1.6, 8.0 Hz, 1H), 4.13-4.02 (m, 2H), 3.47-3.34 (m, 4H), 3.14-3.05 (m, 2H), 2.91-2.79 (m, 2H), 2.07-1.97 (m, 2H), 1.85-1.66 (m, 5H), 1.45-1.29 (m, 1H). m/z: [ESI⁺] 482, 484 (M+H)⁺. (C₂₅H₂₈ClN₅OS).

### 2-(4-(aminomethyl)-2-fluorophenyl)-N-(3-(piperidin-1-yl)propyl)benzo[d]imidazo[2,1-b]thiazole-7-carboxamide (Compound 374)

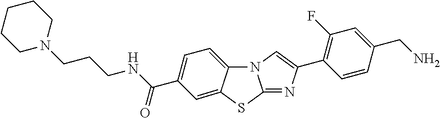 [SMILES: NCc1ccc(-c2cn3c(n2)sc2cc(C(=O)NCCCN4CCCCC4)ccc23)c(F)c1]

Compound 2-(4-(aminomethyl)-2-fluorophenyl)-N-(3-(piperidin-1-yl)propyl)benzo[d]imidazo[2,1-b]thiazole-7-carboxamide was prepared from tert-butyl (3-fluoro-4-(7-((3-(piperidin-1-yl)propyl)carbamoyl)benzo[d]imidazo[2,1-b]thiazol-2-yl)benzyl)carbamate (300 mg, 0.530 mmol) following a similar procedure to that described for the synthesis of 4-amino-i-(4-bromo-3-fluorophenyl)butan-1-one hydrochloride, and was isolated as a white solid.

Yield 20 mg (8%). ¹H NMR (400 MHz, DMSO) δ 8.68 (d, J=3.6 Hz, 1H), 8.64 (t, J=5.6 Hz, 1H), 8.49 (d, J=1.6 Hz, 1H), 8.26 (d, J=8.4 Hz, 1H), 8.08 (t, J=8.0 Hz, 1H), 8.01 (dd, J=1.6, 8.4 Hz, 1H), 7.33 (d, J=12.4 Hz, 1H), 7.26 (dd, J=1.6, 8.0 Hz, 1H), 3.78 (s, 2H), 3.33-3.30 (m, 2H), 2.52-2.50 (m, 2H), 2.39-2.31 (m, 4H), 1.78-1.63 (m, 2H), 1.58-1.40 (m, 4H), 1.43-1.28 (m, 2H). NH₂ protons not observed. ¹⁹F NMR (376 MHz, DMSO) δ −114.11. m/z: [ESI⁺] 466 (M+H)⁺. (C₂₅H₂₈FN₅OS).

### 2-(4-((cyclopropylamino)methyl)-2,5-difluorophenyl)-N-(3-(diethylamino)propyl)benzo[d]imidazo[2,1-b]thiazole-7-carboxamide hemi-formate (Compound 334)

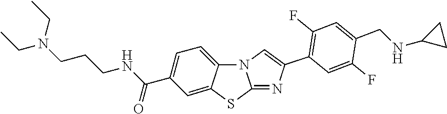 [SMILES: CCN(CC)CCCNC(=O)c1ccc2c(c1)sc1nc(-c3cc(F)c(CNC4CC4)cc3F)cn12]

Compound 2-(4-((cyclopropylamino)methyl)-2,5-difluorophenyl)-N-(3-(diethylamino)propyl)benzo[d]imidazo[2,1-b]thiazole-7-carboxamide hemi-formate was prepared from tert-butyl cyclopropyl(4-(7-((3-(diethylamino)propyl)carbamoyl)benzo[d]imidazo[2,1-b]thiazol-2-yl)-2,5-difluorobenzyl)carbamate (300 mg, 0.490 mmol) following a similar procedure to that described for the synthesis of 4-amino-i-(4-bromo-3-fluorophenyl)butan-1-one hydrochloride, and was isolated as an off-white solid.

Yield 25 mg (9%). ¹H NMR (400 MHz, DMSO) δ 8.75 (d, J=3.6 Hz, 1H), 8.70 (t, J=5.6 Hz, 1H), 8.50 (d, J=1.6 Hz, 1H), 8.28 (d, J=8.4 Hz, 1H), 8.22 (s, 0.68H, formic acid), 8.02 (dd, J=1.6, 8.4 Hz, 1H), 7.79 (dd, J=6.0, 10.4 Hz, 1H), 7.42 (dd, J=6.0, 11.2 Hz, 1H), 3.78 (s, 2H), 3.38-3.26 (m, 2H), 2.78-2.60 (m, 6H), 2.16-2.01 (m, 1H), 1.86-1.69 (m, 2H), 1.04 (t, J=7.2 Hz, 6H), 0.43-0.34 (m, 2H), 0.29-0.22 (m, 2H). Aliphatic NH proton not observed. ¹⁹F NMR (376 MHz, DMSO) δ −119.06, −119.11, −124.25, −124.29. m/z: [ESI⁺] 512 (M+H)⁺. (C₂₇H₃₁F₂N₅OS).

### N-(3-(piperidin-1-yl)propyl)-2-(4-(pyrrolidin-2-yl)phenyl)benzo[d]imidazo[2,1-b]thiazole-7-carboxamide diformate (Compound 373)

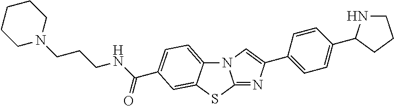 [SMILES: O=C(NCCCN1CCCCC1)c1ccc2c(c1)sc1nc(-c3ccc(C4CCCN4)cc3)cn12]

Compound N-(3-(piperidin-1-yl)propyl)-2-(4-(pyrrolidin-2-yl)phenyl)benzo[d]imidazo[2,1-b]thiazole-7-carboxamide diformate was prepared from tert-butyl 2-(4-(7-((3-(piperidin-1-yl)propyl)carbamoyl)benzo[d]imidazo[2,1-b]thiazol-2-yl)phenyl)pyrrolidine-1-carboxylate (400 mg, 0.681 mmol) following a similar procedure to that described for the synthesis of 4-amino-1-(4-bromo-3-fluorophenyl)butan-1-one hydrochloride, and was isolated as an off-white solid.

Yield 22 mg (6%). ¹H NMR (400 MHz, DMSO) δ 8.85 (s, 1H), 8.70 (t, J=5.6 Hz, 1H), 8.50 (d, J=1.6 Hz, 1H), 8.32 (s, 2H, formic acid), 8.10-8.01 (m, 2H), 7.90 (d, J=8.0 Hz, 2H), 7.54 (d, J=8.0 Hz, 2H), 4.50-4.40 (m, 1H), 3.45-3.13 (m, 4H), 2.50-2.44 (m, 6H), 2.38-2.25 (m, 1H), 2.11-1.86 (m, 3H), 1.80-1.73 (m, 2H), 1.59-1.52 (m, 4H), 1.45-1.35 (m, 2H). Aliphatic NH proton not observed. m/z: [ESI⁺] 488 (M+H)⁺. (C₂₈H₃₃N₅OS).

### (R)—N-(3-(piperidin-1-yl)propyl)-2-(4-(pyrrolidin-2-yl)phenyl)benzo[d]imidazo[2,1-b]thiazole-7-carboxamide hemi-formate (Compound 412)

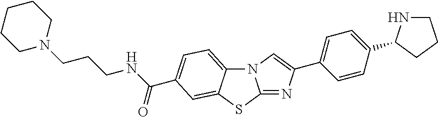 [SMILES: O=C(NCCCN1CCCCC1)c1ccc2c(c1)sc1nc(-c3ccc([C@H]4CCCN4)cc3)cn12]

Compound (R)—N-(3-(piperidin-1-yl)propyl)-2-(4-(pyrrolidin-2-yl)phenyl)benzo[d]imidazo[2,1-b]thiazole-7-carboxamide hemi-formate was prepared from tert-butyl (R)-2-(4-(7-((3-(piperidin-1-yl)propyl)carbamoyl)benzo[d]imidazo[2,1-b]thiazol-2-yl)phenyl)pyrrolidine-1-carboxylate (350 mg, 0.595 mmol) following a similar procedure to that described for the synthesis of 4-amino-1-(4-bromo-3-fluorophenyl)butan-1-one hydrochloride, and was isolated as a light yellow solid.

Yield 141 mg (42%). ¹H NMR (400 MHz, DMSO) δ 8.86 (s, 1H), 8.74 (t, J=5.6 Hz, 1H), 8.51 (s, 1H), 8.32 (s, 1.79H, formic acid), 8.08-8.02 (m, 2H), 7.90 (d, J=8.0 Hz, 2H), 7.56 (d, J=8.0 Hz, 2H), 4.59-4.30 (m, 1H), 3.41-3.14 (m, 4H), 2.62-2.53 (m, 6H), 2.39-2.27 (m, 1H), 2.13-1.91 (m, 3H), 1.89-1.70 (m, 2H), 1.66-1.48 (m, 4H), 1.49-1.35 (m, 2H). Aliphatic NH proton not observed. m/z: [ESI⁺] 488 (M+H)⁺. (C₂₈H₃₃N₅OS).

### (S)—N-(3-(piperidin-1-yl)propyl)-2-(4-(pyrrolidin-2-yl)phenyl)benzo[d]imidazo[2,1-b]thiazole-7-carboxamide diformate (Compound 413)

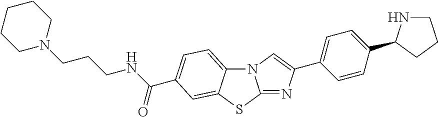 [SMILES: O=C(NCCCN1CCCCC1)c1ccc2c(c1)sc1nc(-c3ccc([C@@H]4CCCN4)cc3)cn12]

Compound (S)—N-(3-(piperidin-1-yl)propyl)-2-(4-(pyrrolidin-2-yl)phenyl)benzo[d]imidazo[2,1-b]thiazole-7-carboxamide diformate was prepared from tert-butyl (S)-2-(4-(7-((3-(piperidin-1-yl)propyl)carbamoyl)benzo[d]imidazo[2,1-b]thiazol-2-yl)phenyl)pyrrolidine-1-carboxylate (350 mg, 0.595 mmol) following a similar procedure to that described for the synthesis of 4-amino-1-(4-bromo-3-fluorophenyl)butan-1-one hydrochloride, and was isolated as a light yellow solid

Yield 107 mg (31%). ¹H NMR (400 MHz, DMSO) δ 8.86 (s, 1H), 8.74 (t, J=5.6 Hz, 1H), 8.51 (s, 1H), 8.32 (s, 2H, formic acid), 8.10-8.00 (m, 2H), 7.90 (d, J=8.0 Hz, 2H), 7.56 (d, J=8.0 Hz, 2H), 4.59-4.30 (m, 1H), 3.41-3.14 (m, 4H), 2.55-2.52 (m, 6H), 2.39-2.27 (m, 1H), 2.13-1.91 (m, 3H), 1.89-1.70 (m, 2H), 1.66-1.48 (m, 4H), 1.49-1.35 (m, 2H). Aliphatic NH proton not observed. m/z: [ESI⁺] 488 (M+H)⁺. (C₂₈H₃₃N₅OS).

### 2-(4-(1-aminocyclopropyl)phenyl)-N-(3-(piperidin-1-yl)propyl)benzo[d]imidazo[2,1-b]thiazole-7-carboxamide (Compound 332)

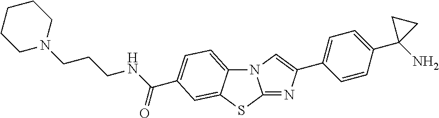 [SMILES: NC1(c2ccc(-c3cn4c(n3)sc3cc(C(=O)NCCCN5CCCCC5)ccc34)cc2)CC1]

Compound 2-(4-(1-aminocyclopropyl)phenyl)-N-(3-(piperidin-1-yl)propyl)benzo[d]imidazo[2,1-b]thiazole-7-carboxamide was prepared from tert-butyl (1-(4-(7-((3-(piperidin-1-yl)propyl)carbamoyl)benzo[d]imidazo[2,1-b]thiazol-2-yl)phenyl)cyclopropyl)carbamate (1.50 g, 2.61 mmol) following a similar procedure to that described for the synthesis of 4-amino-i-(4-bromo-3-fluorophenyl)butan-1-one hydrochloride, and was isolated as a light yellow solid.

Yield 974 mg (79%). ¹H NMR (400 MHz, DMSO) δ 8.77 (s, 1H), 8.64 (t, J=5.6 Hz, 1H), 8.48 (d, J=1.6 Hz, 1H), 8.09-7.98 (m, 2H), 7.77 (d, J=8.4 Hz, 2H), 7.37 (d, J=8.4 Hz, 2H), 3.35-3.25 (m, 2H), 2.41-2.30 (m, 6H), 1.85-1.63 (m, 2H), 1.58-1.43 (m, 4H), 1.43-1.33 (m, 2H), 0.99 (t, J=2.4 Hz, 2H), 0.96 (t, J=2.4 Hz, 2H). NH₂ protons not observe. m/z: [ESI⁺] 474 (M+H)⁺. (C₂₇H₃₁N₅OS).

### 2-(4-(1-aminocyclopropyl)phenyl)-N-(3-(diethylamino)propyl)benzo[d]imidazo[2,1-b]thiazole-7-carboxamide diformate (Compound 318)

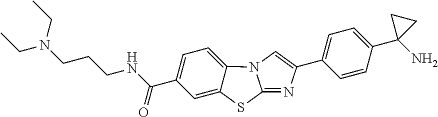 [SMILES: CCN(CC)CCCNC(=O)c1ccc2c(c1)sc1nc(-c3ccc(C4(N)CC4)cc3)cn12]

Compound 2-(4-(1-aminocyclopropyl)phenyl)-N-(3-(diethylamino)propyl)benzo[d]imidazo[2,1-b]thiazole-7-carboxamide diformate was prepared from tert-butyl (1-(4-(7-((3-(diethylamino)propyl)carbamoyl)benzo[d]imidazo[2,1-b]thiazol-2-yl)phenyl)cyclopropyl)carbamate (200 mg, 0.356 mmol) following a similar procedure to that described for the synthesis of 4-amino-1-(4-bromo-3-fluorophenyl)butan-1-one hydrochloride, and was isolated as a brown solid.

Yield 40 mg (20%). ¹H NMR (400 MHz, DMSO) δ 8.78 (s, 1H), 8.68 (t, J=5.6 Hz, 1H), 8.49 (d, J=1.6 Hz, 1H), 8.24 (s, 2H, formic acid), 8.09-7.99 (m, 2H), 7.80 (d, J=8.4 Hz, 2H), 7.39 (d, J=8.4 Hz, 2H), 3.45-3.16 (m, 2H), 2.67-2.62 (m, 6H), 1.81-1.69 (m, 2H), 1.14-0.97 (m, 10H). NH₂ protons not observed. m/z: [ESI⁺] 462 (M+H)⁺. (C₂₆H₃₁N₅OS).

### 2-(3-(aminomethyl)phenyl)-N-(3-(diethylamino)propyl)benzo[d]imidazo[2,1-b]thiazole-7-carboxamide hemi-formate (Compound 299)

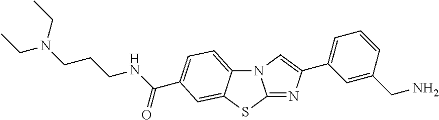 [SMILES: CCN(CC)CCCNC(=O)c1ccc2c(c1)sc1nc(-c3cccc(CN)c3)cn12]

Compound 2-(3-(aminomethyl)phenyl)-N-(3-(diethylamino)propyl)benzo[d]imidazo[2,1-b]thiazole-7-carboxamide hemi-formate was prepared from tert-butyl (3-(7-((3-(diethylamino)propyl)carbamoyl)benzo[d]imidazo[2,1-b]thiazol-2-yl)benzyl)carbamate (400 mg, 0.747 mmol) following a similar procedure to that described for the synthesis of 4-amino-i-(4-bromo-3-fluorophenyl)butan-1-one hydrochloride, and was isolated as a white solid.

Yield 4 mg (1%). ¹H NMR (400 MHz, DMSO) δ 8.96-8.74 (m, 2H), 8.51 (s, 1H), 8.42 (s, 1.67H, formic acid), 8.16-7.95 (m, 3H), 7.83 (d, J=7.6 Hz, 1H), 7.60-7.42 (m, 1H), 7.39 (d, J=7.6 Hz, 1H), 4.15-3.94 (m, 2H), 3.42-3.16 (m, 2H), 2.81-2.63 (m, 6H), 1.97-1.58 (m, 2H), 1.03 (t, J=7.2 Hz, 6H). NH₂ protons not observed. m/z: [ESI⁺] 436 (M+H)⁺. (C₂₄H₂₉N₅OS).

### 2-(4-(aminomethyl)phenyl)-N-(3-(piperidin-1-yl)propyl)benzo[d]imidazo[2,1-b]thiazole-7-carboxamide diformate (Compound 290)

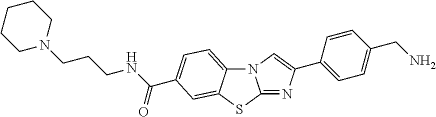 [SMILES: NCc1ccc(-c2cn3c(n2)sc2cc(C(=O)NCCCN4CCCCC4)ccc23)cc1]

Compound 2-(4-(aminomethyl)phenyl)-N-(3-(piperidin-1-yl)propyl)benzo[d]imidazo[2,1-b]thiazole-7-carboxamide diformate was prepared from tert-butyl (4-(7-((3-(piperidin-1-yl)propyl)carbamoyl)benzo[d]imidazo[2,1-b]thiazol-2-yl)benzyl)carbamate (200 mg, 0.365 mmol) following a similar procedure to that described for the synthesis of 4-amino-1-(4-bromo-3-fluorophenyl)butan-1-one hydrochloride, and was isolated as an off-white solid.

Yield 36 mg (18%). ¹H NMR (400 MHz, DMSO) δ 8.84 (s, 1H), 8.67 (t, J=5.6 Hz, 1H), 8.50 (s, 1H), 8.42-8.20 (m, 2H, formic acid), 8.10-7.95 (m, 2H), 7.89 (d, J=7.6 Hz, 2H), 7.51 (d, J=7.6 Hz, 2H), 4.06-3.92 (m, 2H), 3.39-3.31 (m, 2H), 2.43-2.29 (m, 6H), 1.79-1.66 (m, 2H), 1.63-1.48 (m, 4H), 1.47-1.29 (m, 2H). NH₂ protons not observed. m/z: [ESI⁺] 448 (M+H)⁺. (C₂₅H₂₉N₅OS).

### 2-(4-(aminomethyl)-3-fluorophenyl)-N-(3-(piperidin-1-yl)propyl)benzo[d]imidazo[2,1-b]thiazole-7-carboxamide (Compound 375)

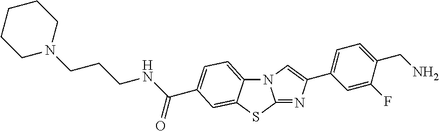 [SMILES: NCc1ccc(-c2cn3c(n2)sc2cc(C(=O)NCCCN4CCCCC4)ccc23)cc1F]

Compound 2-(4-(aminomethyl)-3-fluorophenyl)-N-(3-(piperidin-1-yl)propyl)benzo[d]imidazo[2,1-b]thiazole-7-carboxamide was prepared from tert-butyl (2-fluoro-4-(7-((3-(piperidin-1-yl)propyl)carbamoyl)benzo[d]imidazo[2,1-b]thiazol-2-yl)benzyl)carbamate (100 mg, 0.177 mmol) following a similar procedure to that described for the synthesis of 4-amino-i-(4-bromo-3-fluorophenyl)butan-1-one hydrochloride, and was isolated as an off-white solid.

Yield 26 mg (32%). ¹H NMR (400 MHz, DMSO) δ 8.73 (d, J=2.4 Hz, 1H), 8.42 (s, 1H), 8.36 (t, J=5.6 Hz, 1H), 8.03-7.96 (m, 2H), 7.66 (d, J=8.0 Hz, 1H), 7.61-7.45 (m, 2H), 3.80 (s, 2H), 3.39-3.24 (m, 2H), 2.36-2.33 (m, 6H), 1.78-1.66 (m, 2H), 1.51-1.44 (m, 4H), 1.44-1.32 (m, 2H). NH₂ protons not observed. ¹⁹F NMR (376 MHz, DMSO) δ −119.78. m/z: [ESI⁺] 466 (M+H)⁺. (C₂₅H₂₈FN₅OS).

### 2-(4-(aminomethyl)-2-(trifluoromethyl)phenyl)-N-(3-(piperidin-1-yl)propyl)benzo[d]imidazo[2,1-b]thiazole-7-carboxamide diformate (Compound 372)

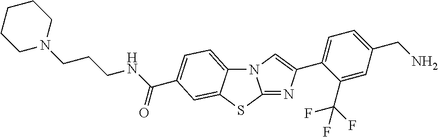 [SMILES: NCc1ccc(-c2cn3c(n2)sc2cc(C(=O)NCCCN4CCCCC4)ccc23)c(C(F)(F)F)c1]

Compound 2-(4-(aminomethyl)-2-(trifluoromethyl)phenyl)-N-(3-(piperidin-1-yl)propyl)benzo[d]imidazo[2,1-b]thiazole-7-carboxamide diformate was prepared from tert-butyl (4-(7-((3-(piperidin-1-yl)propyl)carbamoyl)benzo[d]imidazo[2,1-b]thiazol-2-yl)-3-(trifluoromethyl)benzyl)carbamate (200 mg, 0.325 mmol) following a similar procedure to that described for the synthesis of 4-amino-i-(4-bromo-3-fluorophenyl)butan-1-one hydrochloride, and was isolated as a light yellow solid.

Yield 45 mg (23%). ¹H NMR (400 MHz, DMSO) δ 8.70 (t, J=5.6 Hz, 1H), 8.58-8.50 (m, 2H), 8.31 (s, 2H, formic acid), 8.24 (d, J=8.4 Hz, 1H), 8.03 (dd, J=1.6, 8.4 Hz, 1H), 7.96 (d, J=1.6 Hz, 1H), 7.89 (d, J=8.0 Hz, 1H), 7.79 (d, J=8.0 Hz, 1H), 4.08 (s, 2H), 3.36-3.28 (m, 2H), 2.48-2.43 (m, 6H), 1.79-1.71 (m, 2H), 1.56-1.50 (m, 4H), 1.45-1.35 (m, 2H). NH₂ protons not observed. ¹⁹F NMR (376 MHz, DMSO) δ −56.87. m/z: [ESI⁺] 516 (M+H)⁺. (C₂₆H₂₈F₃N₅OS).

### (R)-2-(2-fluoro-4-(pyrrolidin-2-yl)phenyl)-N-(3-(piperidin-1-yl)propyl)benzo[4,5]thiazolo[3,2-b][1,2,4]triazole-6-carboxamide dihydrochloride (Compound 421)

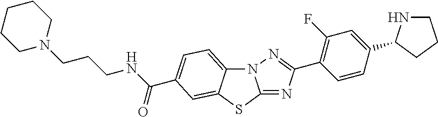 [SMILES: O=C(NCCCN1CCCCC1)c1ccc2c(c1)sc1nc(-c3ccc([C@H]4CCCN4)cc3F)nn12]

Compound (R)-2-(2-fluoro-4-(pyrrolidin-2-yl)phenyl)-N-(3-(piperidin-1-yl)propyl)benzo[4,5]thiazolo[3,2-b][1,2,4]triazole-6-carboxamide dihydrochloride was prepared from tert-butyl (R)-2-(3-fluoro-4-(6-((3-(piperidin-1-yl)propyl)carbamoyl)benzo[4,5]thiazolo[3,2-b][1,2,4]triazol-2-yl)phenyl)pyrrolidine-1-carboxylate (200 mg, 0.330 mmol) following a similar procedure to that described for the synthesis of 4-amino-i-(4-bromo-3-fluorophenyl)butan-1-one hydrochloride, and was isolated as a brown solid.

Yield 78 mg (41%). ¹H NMR (400 MHz, DMSO) δ 10.32 (br s, 1H, NH⁺), 10.21 (br s, 1H, NH⁺), 9.28 (br s, 1H, NH⁺), 8.99 (t, J=5.6 Hz, 1H), 8.76 (d, J=1.2 Hz, 1H), 8.25-8.18 (m, 1H), 8.17-8.15 (m, 2H), 7.70 (dd, J=1.6, 12.0 Hz, 1H), 7.57 (dd, J=1.6, 8.0 Hz, 1H), 4.69-4.61 (m, 1H), 3.47-3.27 (m, 6H), 3.14-3.04 (m, 2H), 2.93-2.79 (m, 2H), 2.48-2.41 (m, 1H), 2.19-1.95 (m, 5H), 1.84-1.74 (m, 4H), 1.73-1.66 (m, 1H), 1.47-1.31 (m, 1H). ¹⁹F NMR (376 MHz, DMSO) δ −110.54. m/z: [ESI⁺] 507 (M+H)⁺. (C₂₇H₃₁FN₆OS).

### (S)-2-(2-fluoro-4-(pyrrolidin-2-yl)phenyl)-N-(3-(piperidin-1-yl)propyl)benzo[4,5]thiazolo[3,2-b][1,2,4]triazole-6-carboxamide dihydrochloride (Compound 422)

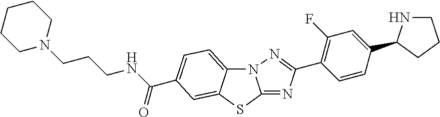 [SMILES: O=C(NCCCN1CCCCC1)c1ccc2c(c1)sc1nc(-c3ccc([C@@H]4CCCN4)cc3F)nn12]

Compound (S)-2-(2-fluoro-4-(pyrrolidin-2-yl)phenyl)-N-(3-(piperidin-1-yl)propyl)benzo[4,5]thiazolo[3,2-b][1,2,4]triazole-6-carboxamide dihydrochloride was prepared from tert-butyl (S)-2-(3-fluoro-4-(6-((3-(piperidin-1-yl)propyl)carbamoyl)benzo[4,5]thiazolo[3,2-b][1,2,4]triazol-2-yl)phenyl)pyrrolidine-1-carboxylate (200 mg, 0.330 mmol) following a similar procedure to that described for the synthesis of 4-amino-i-(4-bromo-3-fluorophenyl)butan-1-one hydrochloride, and was isolated as a brown solid.

Yield 63 mg (33%). ¹H NMR (400 MHz, DMSO) δ 10.53-10.24 (m, 2H, NH₂+), 9.35 (br s, 1H, NH⁺), 9.00 (t, J=5.6 Hz, 1H), 8.77 (d, J=1.6 Hz, 1H), 8.26-8.11 (m, 3H), 7.71 (dd, J=2.0, 12.0 Hz, 1H), 7.57 (dd, J=1.6, 8.0 Hz, 1H), 4.72-4.59 (m, 1H), 3.48-3.31 (m, 6H), 3.14-3.04 (m, 2H), 2.92-2.79 (m, 2H), 2.49-2.43 (m, 1H), 2.22-1.97 (m, 5H), 1.85-1.75 (m, 4H), 1.73-1.64 (m, 1H), 1.46-1.29 (m, 1H). ¹⁹F NMR (376 MHz, DMSO) δ −110.54. m/z: [ESI⁺] 507 (M+H)⁺. (C₂₇H₃₁FN₆OS).

### 2-(2-fluoro-4-(methylcarbamoyl)phenyl)-N-(piperidin-4-yl)benzo[d]imidazo[2,1-b]thiazole-7-carboxamide (Compound 363)

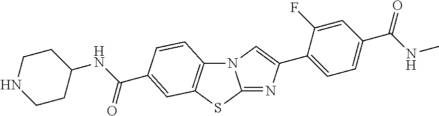 [SMILES: CNC(=O)c1ccc(-c2cn3c(n2)sc2cc(C(=O)NC4CCNCC4)ccc23)c(F)c1]

Compound 2-(2-fluoro-4-(methylcarbamoyl)phenyl)-N-(piperidin-4-yl)benzo[d]imidazo[2,1-b]thiazole-7-carboxamide was prepared from tert-butyl 4-(2-(2-fluoro-4-(methylcarbamoyl)phenyl)benzo[d]imidazo[2,1-b]thiazole-7-carboxamido)piperidine-1-carboxylate (270 mg, 0.489 mmol) following a similar procedure to that described for the synthesis of 4-amino-i-(4-bromo-3-fluorophenyl)butan-1-one hydrochloride, and was isolated as a white solid.

Yield 9 mg (4%). ¹H NMR (400 MHz, DMSO) δ 8.83 (d, J=3.6 Hz, 1H), 8.57 (q, J=4.4 Hz, 1H), 8.51 (s, 1H), 8.40 (d, J=7.6 Hz, 1H), 8.29 (d, J=8.4 Hz, 1H), 8.26-8.20 (m, 1H), 8.05 (d, J=8.4 Hz, 1H), 7.82-7.73 (m, 2H), 3.99 (br s, 1H), 3.92-3.85 (m, 1H), 3.01-2.96 (m, 2H), 2.82 (d, J=4.4 Hz, 3H), 2.57-2.54 (m, 2H), 1.79-1.75 (m, 2H), 1.49-1.39 (m, 2H). ¹⁹F NMR (376 MHz, DMSO) δ −113.11. m/z: [ESI⁺]452 (M+H)⁺. (C₂₃H₂₂FN₅O₂S).

### N-(2-(2-fluoro-4-(methylcarbamoyl)phenyl)benzo[d]imidazo[2,1-b]thiazol-7-yl)piperidine-4-carboxamide (Compound 364)

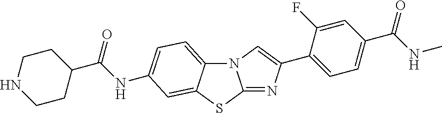 [SMILES: CNC(=O)c1ccc(-c2cn3c(n2)sc2cc(NC(=O)C4CCNCC4)ccc23)c(F)c1]

Compound N-(2-(2-fluoro-4-(methylcarbamoyl)phenyl)benzo[d]imidazo[2,1-b]thiazol-7-yl)piperidine-4-carboxamide was prepared from tert-butyl 4-((2-(2-fluoro-4-(methylcarbamoyl)phenyl)benzo[d]imidazo[2,1-b]thiazol-7-yl)carbamoyl)piperidine-1-carboxylate (300 mg, 0.544 mmol) following a similar procedure to that described for the synthesis of 4-amino-1-(4-bromo-3-fluorophenyl)butan-1-one hydrochloride, and was isolated as a white solid.

Yield 25.7 mg (10%). ¹H NMR (400 MHz, DMSO) δ 10.35 (s, 1H), 8.70-8.68 (m, 1H), 8.58-8.54 (m, 1H), 8.41-8.34 (m, 2H), 8.21 (t, J=8.0 Hz, 1H), 8.12 (d, J=8.8 Hz, 1H), 7.82-7.73 (m, 2H), 7.67 (d, J=8.8 Hz, 1H), 3.25-3.18 (m, 2H), 2.81 (d, J=4.4 Hz, 3H), 2.78-2.73 (m, 2H), 2.63-2.57 (m, 1H), 1.91-1.85 (m, 2H), 1.79-1.70 (m, 2H). m/z: [ESI⁺] 452 (M+H)⁺. (C₂₃H₂₂FN₅O₂S).

### 2-(4-(aminomethyl)-2-fluorophenyl)-N-(3-(piperidin-1-yl)propyl)benzo[4,5]thiazolo[3,2-b][1,2,4]triazole-6-carboxamide hemi-formate (Compound 387)

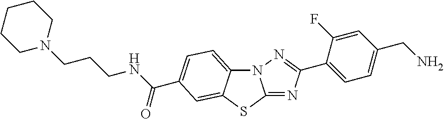 [SMILES: NCc1ccc(-c2nc3sc4cc(C(=O)NCCCN5CCCCC5)ccc4n3n2)c(F)c1]

Compound 2-(4-(aminomethyl)-2-fluorophenyl)-N-(3-(piperidin-1-yl)propyl)benzo[4,5]thiazolo[3,2-b][1,2,4]triazole-6-carboxamide hemi-formate was prepared from tert-butyl (3-fluoro-4-(6-((3-(piperidin-1-yl)propyl)carbamoyl)benzo[4,5]thiazolo[3,2-b][1,2,4]triazol-2-yl)benzyl)carbamate (100 mg, 0.176 mmol) following a similar procedure to that described for the synthesis of 4-amino-I-(4-bromo-3-fluorophenyl)butan-1-one hydrochloride, and was isolated as a light yellow solid.

Yield 32 mg (34%). ¹H NMR (400 MHz, DMSO) δ 8.75 (t, J=5.6 Hz, 1H), 8.68 (d, J=1.6 Hz, 1H), 8.28 (s, 1.47H, formic acid), 8.19-8.07 (m, 3H), 7.47 (d, J=12.0 Hz, 1H), 7.40 (d, J=8.0 Hz, 1H), 3.96 (s, 2H), 3.39-3.28 (m, 2H), 2.43-2.35 (m, 6H), 1.80-1.67 (m, 2H), 1.57-1.47 (m, 4H), 1.43-1.35 (m, 2H). NH₂ protons not observed ¹⁹F NMR (376 MHz, DMSO) δ −111.56. m/z: [ESI⁺] 467 (M+H)⁺ (C₂₄H₂₇FN₆OS).

### 2-(4-(aminomethyl)-2-fluorophenyl)-N-(3-(diethylamino)propyl)benzo[4,5]thiazolo[3,2-b][1,2,4]triazole-6-carboxamide hemi-formate (Compound 388)

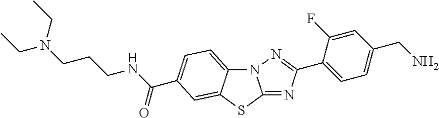 [SMILES: CCN(CC)CCCNC(=O)c1ccc2c(c1)sc1nc(-c3ccc(CN)cc3F)nn12]

Compound 2-(4-(aminomethyl)-2-fluorophenyl)-N-(3-(diethylamino)propyl)benzo[4,5]thiazolo[3,2-b][1,2,4]triazole-6-carboxamide hemi-formate was prepared from tert-butyl (4-(6-((3-(diethylamino)propyl)carbamoyl)benzo[4,5]thiazolo[3,2-b][1,2,4]triazol-2-yl)-3-fluorobenzyl)carbamate (160 mg, 0.288 mmol) following a similar procedure to that described for the synthesis of 4-amino-i-(4-bromo-3-fluorophenyl)butan-1-one hydrochloride, and was isolated as a light yellow solid.

Yield 61 mg (40%). ¹H NMR (400 MHz, DMSO) δ 8.80 (t, J=5.6 Hz, 1H), 8.70 (s, 1H), 8.28 (s, 1.49H, formic acid), 8.21-8.08 (m, 3H), 7.53 (d, J=12.0 Hz, 1H), 7.44 (d, J=8.0 Hz, 1H), 4.04 (s, 2H), 3.40-3.30 (m, 2H), 2.69-2.58 (m, 6H), 1.79-1.70 (m, 2H), 1.03 (t, J=7.2 Hz, 6H). NH₂ protons not observed. ¹⁹F NMR (376 MHz, DMSO) δ −111.35. m/z: [ESI⁺] 455 (M+H)⁺ (C₂₃H₂₇FN₆OS).

### 2-(4-(aminomethyl)-2-fluorophenyl)-N-(3-(4-fluoropiperidin-1-yl)propyl)benzo[4,5]thiazolo[3,2-b][1,2,4]triazole-6-carboxamide diformate (Compound 390)

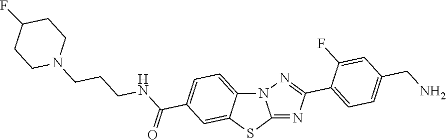 [SMILES: NCc1ccc(-c2nc3sc4cc(C(=O)NCCCN5CCC(F)CC5)ccc4n3n2)c(F)c1]

Compound 2-(4-(aminomethyl)-2-fluorophenyl)-N-(3-(4-fluoropiperidin-1-yl)propyl)benzo[4,5]thiazolo[3,2-b][1,2,4]triazole-6-carboxamide diformate was prepared from tert-butyl (3-fluoro-4-(6-((3-(4-fluoropiperidin-1-yl)propyl)carbamoyl)benzo[4,5]thiazolo[3,2-b][1,2,4]triazol-2-yl)benzyl)carbamate (200 mg, 0.342 mmol) following a similar procedure to that described for the synthesis of 4-amino-i-(4-bromo-3-fluorophenyl)butan-1-one hydrochloride, and was isolated as a light yellow solid.

Yield 16 mg (8%). ¹H NMR (400 MHz, DMSO) δ 8.79-8.57 (m, 2H), 8.30 (s, 2H, formic acid), 8.23-7.94 (m, 3H), 7.55-7.36 (m, 2H), 4.77-4.55 (m, 1H), 4.12-3.98 (m, 2H), 3.45-3.22 (m, 2H), 2.56-2.52 (m, 2H), 2.43-2.19 (m, 4H), 1.94-1.78 (m, 2H), 1.78-1.63 (m, 4H). NH₂ protons not observed. ¹⁹F NMR (376 MHz, DMSO) δ −111.26. ¹⁹F NMR signal of 4-fluoropiperidine was not observed. m/z: [ESI⁺] 485 (M+H)⁺ (C₂₄H₂₆F₂N₆OS).

### 2-(4-((cyclopropylamino)methyl)phenyl)-N-(3-(piperidin-1-yl)propyl)benzo[4,5]thiazolo[3,2-b][1,2,4]triazole-6-carboxamide (Compound 393)

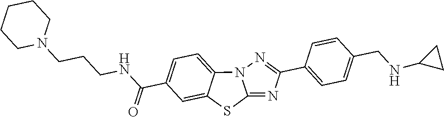 [SMILES: O=C(NCCCN1CCCCC1)c1ccc2c(c1)sc1nc(-c3ccc(CNC4CC4)cc3)nn12]

Compound 2-(4-((cyclopropylamino)methyl)phenyl)-N-(3-(piperidin-1-yl)propyl)benzo[4,5]thiazolo[3,2-b][1,2,4]triazole-6-carboxamide was prepared from tert-butyl cyclopropyl(4-(6-((3-(piperidin-1-yl)propyl)carbamoyl)benzo[4,5]thiazolo[3,2-b][1,2,4]triazol-2-yl)benzyl)carbamate (250 mg, 0.425 mmol) following a similar procedure to that described for the synthesis of 4-amino-i-(4-bromo-3-fluorophenyl)butan-1-one hydrochloride, and was isolated as a light yellow solid.

Yield 36 mg (17%). ¹H NMR (400 MHz, DMSO) δ 8.71 (t, J=5.6 Hz, 1H), 8.65 (s, 1H), 8.21-8.03 (m, 4H), 7.50 (d, J=8.0 Hz, 2H), 3.80 (s, 2H), 3.36-3.33 (m, 2H), 2.77 (br s, 1H), 2.38-2.27 (m, 6H), 2.13-2.05 (m, 1H), 1.75-1.65 (m, 2H), 1.54-1.45 (m, 4H), 1.42-1.32 (m, 2H), 0.40-0.33 (m, 2H), 0.30-0.25 (m, 2H). m/z: [ESI⁺] 489 (M+H)⁺ (C₂₇H₃₂N₆OS).

### 2-(4-((cyclopropylamino)methyl)phenyl)-N-(3-(diethylamino)propyl)benzo[4,5]thiazolo[3,2-b][1,2,4]triazole-6-carboxamide (Compound 394)

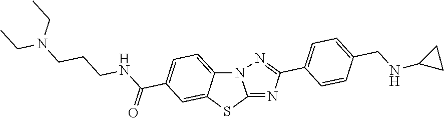 [SMILES: CCN(CC)CCCNC(=O)c1ccc2c(c1)sc1nc(-c3ccc(CNC4CC4)cc3)nn12]

Compound 2-(4-((cyclopropylamino)methyl)phenyl)-N-(3-(diethylamino)propyl)benzo[4,5]thiazolo[3,2-b][1,2,4]triazole-6-carboxamide was prepared from tert-butyl cyclopropyl(4-(6-((3-(diethylamino)propyl)carbamoyl)benzo[4,5]thiazolo[3,2-b][1,2,4]triazol-2-yl)benzyl)carbamate (300 mg, 0.520 mmol) following a similar procedure to that described for the synthesis of 4-amino-i-(4-bromo-3-fluorophenyl)butan-1-one hydrochloride, and was isolated as an off-white solid.

Yield 70 mg (28%). ¹H NMR (400 MHz, DMSO) δ 8.70 (t, J=5.6 Hz, 1H), 8.65 (s, 1H), 8.15-8.06 (m, 4H), 7.50 (d, J=8.0 Hz, 2H), 3.80 (s, 2H), 3.36-3.30 (m, 2H), 2.75 (br s, 1H), 2.55-2.40 (m, 6H), 2.12-2.05 (m, 1H), 1.72-1.63 (m, 2H), 0.96 (t, J=7.2 Hz, 6H), 0.40-0.32 (m, 2H), 0.30-0.24 (m, 2H). m/z: [ESI⁺] 477 (M+H)⁺ (C₂₆H₃₂N₆OS).

### 2-(4-((cyclopropylamino)methyl)-2-fluorophenyl)-N-(3-(diethylamino)propyl)benzo[4,5]thiazolo[3,2-b][1,2,4]triazole-6-carboxamide (Compound 395)

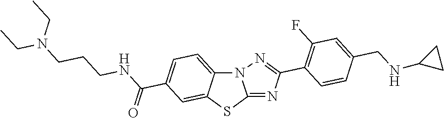 [SMILES: CCN(CC)CCCNC(=O)c1ccc2c(c1)sc1nc(-c3ccc(CNC4CC4)cc3F)nn12]

Compound 2-(4-((cyclopropylamino)methyl)-2-fluorophenyl)-N-(3-(diethylamino)propyl)benzo[4,5]thiazolo[3,2-b][1,2,4]triazole-6-carboxamide was prepared from tert-butyl cyclopropyl(4-(6-((3-(diethylamino)propyl)carbamoyl)benzo[4,5]thiazolo[3,2-b][1,2,4]triazol-2-yl)-3-fluorobenzyl)carbamate (150 mg, 0.252 mmol) following a similar procedure to that described for the synthesis of 4-amino-1-(4-bromo-3-fluorophenyl)butan-1-one hydrochloride, and was isolated as a light yellow solid.

Yield 30 mg (24%). ¹H NMR (400 MHz, DMSO) δ 8.71 (t, J=5.6 Hz, 1H), 8.65 (d, J=1.6 Hz, 1H), 8.14-8.00 (m, 3H), 7.42-7.24 (m, 2H), 3.80 (s, 2H), 3.31-3.22 (m, 2H), 2.91 (br s, 1H), 2.53-2.39 (m, 6H), 2.11-2.00 (m, 1H), 1.73-1.62 (m, 2H), 0.98 (d, J=7.2 Hz, 6H), 0.42-0.31 (m, 2H), 0.31-0.22 (m, 2H). ¹⁹F NMR (376 MHz, DMSO) δ −112.08. m/z: [ESI⁺] 495 (M+H)⁺. (C₂₆H₃₁FN₆OS).

### 2-(4-((cyclopropylamino)methyl)-2-fluorophenyl)-N-(3-(piperidin-1-yl)propyl)benzo[4,5]thiazolo[3,2-b][1,2,4]triazole-6-carboxamide (Compound 396)

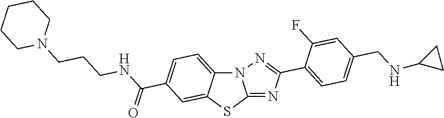 [SMILES: O=C(NCCCN1CCCCC1)c1ccc2c(c1)sc1nc(-c3ccc(CNC4CC4)cc3F)nn12]

Compound 2-(4-((cyclopropylamino)methyl)-2-fluorophenyl)-N-(3-(piperidin-1-yl)propyl)benzo[4,5]thiazolo[3,2-b][1,2,4]triazole-6-carboxamide was prepared from tert-butyl cyclopropyl(3-fluoro-4-(6-((3-(piperidin-1-yl)propyl)carbamoyl)benzo[4,5]thiazolo[3,2-b][1,2,4]triazol-2-yl)benzyl)carbamate (300 mg, 0.494 mmol) following a similar procedure to that described for the synthesis of 4-amino-i-(4-bromo-3-fluorophenyl)butan-1-one hydrochloride, and was isolated as alight yellow solid.

Yield 107 mg (43%). ¹H NMR (400 MHz, DMSO) δ 8.83-8.54 (m, 2H), 8.24-7.94 (m, 3H), 7.43-7.26 (m, 2H), 3.94-2.79 (m, 2H), 2.95-2.77 (m, 2H), 2.45-2.28 (m, 6H), 2.17-2.00 (m, 1H), 1.75-1.63 (m, 2H), 1.60-1.43 (m, 4H), 1.41-1.28 (m, 2H), 0.42-0.33 (m, 2H), 0.30-0.18 (m, 2H). Aliphatic NH proton not observed. ¹⁹F NMR (376 MHz, DMSO) δ −112.09. m/z: [ESI⁺]507 (M+H)⁺. (C₂₇H₃₁FN₆OS).

### 2-(4-(1-aminocyclopropyl)phenyl)-N-(3-(diethylamino)propyl)benzo[4,5]thiazolo[3,2-b][1,2,4]triazole-6-carboxamide hemi-formate (Compound 397)

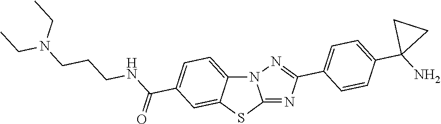 [SMILES: CCN(CC)CCCNC(=O)c1ccc2c(c1)sc1nc(-c3ccc(C4(N)CC4)cc3)nn12]

Compound 2-(4-(1-aminocyclopropyl)phenyl)-N-(3-(diethylamino)propyl)benzo[4,5]thiazolo[3,2-b][1,2,4]triazole-6-carboxamide hemi-formate was prepared from tert-butyl (1-(4-(6-((3-(diethylamino)propyl)carbamoyl)benzo[4,5]thiazolo[3,2-b][1,2,4]triazol-2-yl)phenyl)cyclopropyl)carbamate (195 mg, 0.347 mmol) following a similar procedure to that described for the synthesis of 4-amino-1-(4-bromo-3-fluorophenyl)butan-1-one hydrochloride, and was isolated as an off-white solid.

Yield 37 mg (20%). ¹H NMR (400 MHz, DMSO) δ 8.76 (t, J=5.6 Hz, 1H), 8.66 (s, 1H), 8.23 (s, 1.81H, formic acid), 8.16-8.10 (m, 2H), 8.08 (d, J=8.4 Hz, 2H), 7.48 (d, J=8.4 Hz, 2H), 3.41-3.30 (m, 2H), 2.70-2.60 (m, 6H), 1.82-1.69 (m, 2H), 1.14-0.96 (m, 10H). NH₂ protons not observed. m/z: [ESI⁺] 463 (M+H)⁺. (C₂₅H₃₀N₆OS).

### 2-(4-(1-aminocyclopropyl)phenyl)-N-(3-(piperidin-1-yl)propyl)benzo[4,5]thiazolo[3,2-b][1,2,4]triazole-6-carboxamide hemi-formate (Compound 398)

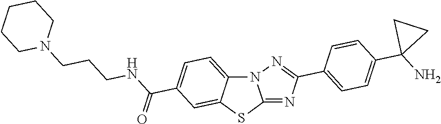 [SMILES: NC1(c2ccc(-c3nc4sc5cc(C(=O)NCCCN6CCCCC6)ccc5n4n3)cc2)CC1]

Compound 2-(4-(1-aminocyclopropyl)phenyl)-N-(3-(piperidin-1-yl)propyl)benzo[4,5]thiazolo[3,2-b][1,2,4]triazole-6-carboxamide hemi-formate was prepared from tert-butyl (1-(4-(6-((3-(piperidin-1-yl)propyl)carbamoyl)benzo[4,5]thiazolo[3,2-b][1,2,4]triazol-2-yl)phenyl)cyclopropyl)carbamate (200 mg, 0.348 mmol) following a similar procedure to that described for the synthesis of 4-amino-1-(4-bromo-3-fluorophenyl)butan-1-one hydrochloride, and was isolated as an off-white solid.

Yield 75 mg (39%). ¹H NMR (400 MHz, DMSO) δ 8.73 (t, J=5.6 Hz, 1H), 8.65 (d, J=1.6 Hz, 1H), 8.21 (s, 1.69H, formic acid), 8.14-8.10 (m, 2H), 8.08 (d, J=8.4 Hz, 2H), 7.48 (d, J=8.4 Hz, 2H), 3.38-3.31 (m, 2H), 2.50-2.37 (m, 6H), 1.79-1.67 (m, 2H), 1.58-1.50 (m, 4H), 1.44-1.36 (m, 2H), 1.09-1.05 (m, 2H), 1.05-1.02 (m, 2H). NH₂ protons not observed. m/z: [ESI⁺] 475 (M+H)⁺. (C₂₆H₃₀N₆OS).

### 2-(4-(aminomethyl)phenyl)-N-(3-(diethylamino)propyl)benzo[4,5]thiazolo[3,2-b][1,2,4]triazole-6-carboxamide dihydrochloride (Compound 348)

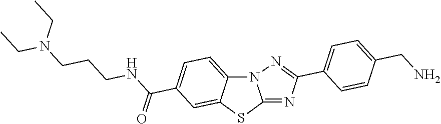 [SMILES: CCN(CC)CCCNC(=O)c1ccc2c(c1)sc1nc(-c3ccc(CN)cc3)nn12]

Compound 2-(4-(aminomethyl)phenyl)-N-(3-(diethylamino)propyl)benzo[4,5]thiazolo[3,2-b][1,2,4]triazole-6-carboxamide dihydrochloride was prepared from tert-butyl (4-(6-((3-(diethylamino)propyl)carbamoyl)benzo[4,5]thiazolo[3,2-b][1,2,4]triazol-2-yl)benzyl)carbamate (80 mg, 0.149 mmol) following a similar procedure to that described for the synthesis of 4-amino-1-(4-bromo-3-fluorophenyl)butan-1-one hydrochloride, and was isolated as an off-white solid.

Yield 43 mg (57%). ¹H NMR (400 MHz, DMSO) δ 10.51 (br s, 1H, NH⁺), 9.00 (t, J=5.6 Hz, 1H), 8.76 (d, J=1.6 Hz, 1H), 8.58 (br s, 3H, NH₃*), 8.19 (d, J=8.0 Hz, 2H), 8.18-8.17 (m, 1H), 8.13 (d, J=8.4 Hz, 1H), 7.69 (d, J=8.0 Hz, 2H), 4.16-4.05 (m, 2H), 3.49-3.35 (m, 2H), 3.20-3.03 (m, 6H), 2.08-1.91 (m, 2H), 1.23 (t, J=7.2 Hz, 6H). m/z: [ESI⁺] 437 (M+H)⁺. (C₂₃H₂₈N₆OS).

### 2-(4-(aminomethyl)phenyl)-N-(3-(piperidin-1-yl)propyl)benzo[4,5]thiazolo[3,2-b][1,2,4]triazole-6-carboxamide (Compound 392)

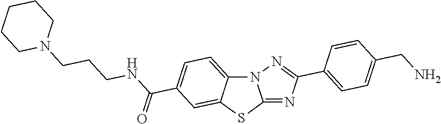 [SMILES: NCc1ccc(-c2nc3sc4cc(C(=O)NCCCN5CCCCC5)ccc4n3n2)cc1]

Compound 2-(4-(aminomethyl)phenyl)-N-(3-(piperidin-1-yl)propyl)benzo[4,5]thiazolo[3,2-b][1,2,4]triazole-6-carboxamide was prepared from tert-butyl (4-(6-((3-(piperidin-1-yl)propyl)carbamoyl)benzo[4,5]thiazolo[3,2-b][1,2,4]triazol-2-yl)benzyl)carbamate (270 mg, 0.492 mmol) following a similar procedure to that described for the synthesis of 4-amino-1-(4-bromo-3-fluorophenyl)butan-1-one hydrochloride, and was isolated as an off-white solid.

Yield 26 mg (12%). ¹H NMR (400 MHz, DMSO) δ 8.70 (t, J=5.6 Hz, 1H), 8.65 (s, 1H), 8.17-8.06 (m, 4H), 7.51 (d, J=8.0 Hz, 2H), 3.81 (s, 2H), 3.36-3.30 (m, 2H), 2.40-2.26 (m, 6H), 1.78-1.65 (m, 2H), 1.56-1.45 (m, 4H), 1.43-1.32 (m, 2H). NH₂ protons not observed. m/z: [ESI⁺] 449 (M+H)⁺. (C₂₄H₂₈N₆OS).

### N-(3-(diethylamino)propyl)-2-(4-(tetrahydro-2H-pyran-4-yl)phenyl)benzo[d]imidazo[2,1-b]thiazole-7-carboxamide (Compound 314)

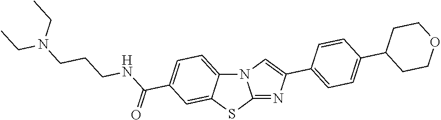 [SMILES: CCN(CC)CCCNC(=O)c1ccc2c(c1)sc1nc(-c3ccc(C4CCOCC4)cc3)cn12]

Compound N-(3-(diethylamino)propyl)-2-(4-(tetrahydro-2H-pyran-4-yl)phenyl)benzo[d]imidazo[2,1-b]thiazole-7-carboxamide was prepared from 2-bromo-N-(3-(diethylamino)propyl)benzo[d]imidazo[2,1-b]thiazole-7-carboxamide (200 mg, 0.489 mmol) and (4-(tetrahydro-2H-pyran-4-yl)phenyl)boronic acid (202 mg, 0.980 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as an off-white solid.

Yield 36 mg (15%). ¹H NMR (400 MHz, DMSO) δ 8.77 (s, 1H), 8.62 (t, J=5.6 Hz, 1H), 8.48 (d, J=1.6 Hz, 1H), 8.06 (d, J=8.4 Hz, 1H), 8.01 (dd, J=1.6, 8.4 Hz, 1H), 7.81 (d, J=8.4 Hz, 2H), 7.36 (d, J=8.4 Hz, 2H), 4.06-3.88 (m, 2H), 3.55-3.40 (m, 2H), 3.32-3.29 (m, 2H), 2.89-2.73 (m, 1H), 2.49-2.40 (m, 6H), 1.77-1.58 (m, 6H), 0.95 (t, J=7.2 Hz, 6H). m/z: [ESI⁺] 491 (M+H)⁺. (C₂₈H₃₄N₄O₂S).

### N-(3-(diethylamino)propyl)-2-(4-morpholinophenyl)benzo[d]imidazo[2,1-b]thiazole-7-carboxamide hemi-formate (Compound 307)

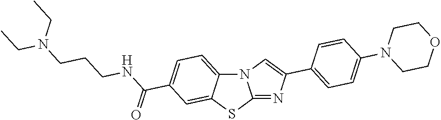 [SMILES: CCN(CC)CCCNC(=O)c1ccc2c(c1)sc1nc(-c3ccc(N4CCOCC4)cc3)cn12]

Compound N-(3-(diethylamino)propyl)-2-(4-morpholinophenyl)benzo[d]imidazo[2,1-b]thiazole-7-carboxamide hemi-formate was prepared from 2-bromo-N-(3-(diethylamino)propyl)benzo[d]imidazo[2,1-b]thiazole-7-carboxamide (300 mg, 0.733 mmol) and (4-morpholinophenyl)boronic acid (300 mg, 1.449 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a brown solid.

Yield 74 mg (19%). ¹H NMR (400 MHz, DMSO) δ 8.69 (t, J=5.6 Hz, 1H), 8.64 (d, J=1.6 Hz, 1H), 8.48 (s, 1H), 8.28-8.15 (m, 0.75H, formic acid), 8.06-8.00 (m, 2H), 7.73 (d, J=8.4 Hz, 2H), 7.02 (d, J=8.4 Hz, 2H), 3.85-3.66 (m, 4H), 3.36-3.35 (m, 2H), 3.27-3.10 (m, 4H), 2.82-2.68 (m, 6H), 1.83-1.72 (m, 2H), 1.06 (t, J=7.2 Hz, 6H). m/z: [ESI⁺] 492 (M+H)⁺. (C₂₇H₃₃N₅O₂S).

### 2-(2-fluoro-4-(methylcarbamoyl)phenyl)-N-(3-(piperidin-1-yl)propyl)benzo[d]imidazo[2,1-b]thiazole-7-carboxamide (Compound 281)

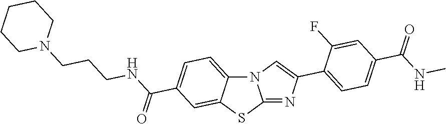 [SMILES: CNC(=O)c1ccc(-c2cn3c(n2)sc2cc(C(=O)NCCCN4CCCCC4)ccc23)c(F)c1]

Compound 2-(2-fluoro-4-(methylcarbamoyl)phenyl)-N-(3-(piperidin-1-yl)propyl)benzo[d]imidazo[2,1-b]thiazole-7-carboxamide was prepared from 2-bromo-N-(3-(piperidin-1-yl)propyl)benzo[d]imidazo[2,1-b]thiazole-7-carboxamide (300 mg, 0.712 mmol) and (2-fluoro-4-(methylcarbamoyl)phenyl)boronic acid (210 mg, 1.066 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as a white solid.

Yield 13 mg (4%). ¹H NMR (400 MHz, DMSO) δ 8.84-8.78 (m, 1H), 8.67 (s, 1H), 8.57 (q, J=5.6 Hz, 1H), 8.52-8.45 (m, 1H), 8.29 (t, J=7.6 Hz, 1H), 8.26-8.21 (m, 1H), 8.02 (dd, J=2.0, 8.4 Hz, 1H), 7.83-7.70 (m, 2H), 3.36-3.32 (m, 2H), 2.81 (d, J=5.6 Hz, 3H), 2.45-2.38 (m, 6H), 1.80-1.68 (m, 2H), 1.56-1.50 (m, 4H), 1.42-1.36 (m, 2H). ¹⁹F NMR (376 MHz, DMSO) δ −113.08. m/z: [ESI⁺] 494 (M+H)⁺. (C₂₆H₂₈FN₅O₂S).

### N-(3-(piperidin-1-yl)propyl)-2-(pyridin-4-yl)benzo[d]imidazo[2,1-b]thiazole-7-carboxamide (Compound 306)

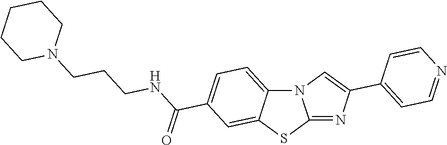 [SMILES: O=C(NCCCN1CCCCC1)c1ccc2c(c1)sc1nc(-c3ccncc3)cn12]

Compound N-(3-(piperidin-1-yl)propyl)-2-(pyridin-4-yl)benzo[d]imidazo[2,1-b]thiazole-7-carboxamide was prepared from 2-bromo-N-(3-(piperidin-1-yl)propyl)benzo[d]imidazo[2,1-b]thiazole-7-carboxamide (200 mg, 0.475 mmol) and pyridin-4-ylboronic acid (117 mg, 0.952 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as an off-white solid

Yield 27 mg (14%). ¹H NMR (400 MHz, DMSO) δ 9.09 (s, 1H), 8.66 (t, J=5.6 Hz, 1H), 8.63 (d, J=6.0 Hz, 2H), 8.51 (d, J=1.6 Hz, 1H), 8.09 (d, J=8.4 Hz, 1H), 8.04 (dd, J=1.6, 8.4 Hz, 1H), 7.81 (d, J=6.0 Hz, 2H), 3.32-3.30 (m, 2H), 2.43-2.32 (m, 6H), 1.82-1.67 (m, 2H), 1.60-1.45 (m, 4H), 1.45-1.34 (m, 2H). m/z: [ESI⁺] 420 (M+H)⁺. (C₂₃H₂₅N₅OS).

### 2-(4-(oxetan-3-yl)phenyl)-N-(3-(piperidin-1-yl)propyl)benzo[d]imidazo[2,1-b]thiazole-7-carboxamide (Compound 315)

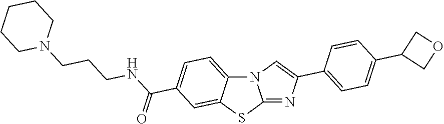 [SMILES: O=C(NCCCN1CCCCC1)c1ccc2c(c1)sc1nc(-c3ccc(C4COC4)cc3)cn12]

Compound 2-(4-(oxetan-3-yl)phenyl)-N-(3-(piperidin-1-yl)propyl)benzo[d]imidazo[2,1-b]thiazole-7-carboxamide was prepared from 2-bromo-N-(3-(piperidin-1-yl)propyl)benzo[d]imidazo[2,1-b]thiazole-7-carboxamide (300 mg, 0.712 mmol) and 4,4,5,5-tetramethyl-2-(4-(oxetan-3-yl)phenyl)-1,3,2-dioxaborolane (371 mg, 1.426 mmol) following a similar procedure to that described for the synthesis of tert-butyl (3-cyclopropyl-4-(7-((3-(piperidin-1-yl)propyl)carbamoyl)benzo[d]imidazo[2,1-b]thiazol-2-yl)benzyl)carbamate, and was isolated as an off-white solid

Yield 61 mg (18%). ¹H NMR (400 MHz, DMSO) δ 8.82 (s, 1H), 8.65 (t, J=5.6 Hz, 1H), 8.48 (d, J=1.6 Hz, 1H), 8.06 (d, J=8.4 Hz, 1H), 8.02 (dd, J=1.6, 8.4 Hz, 1H), 7.88 (d, J=8.4 Hz, 2H), 7.49 (d, J=8.4 Hz, 2H), 4.97 (dd, J=6.0, 8.4 Hz, 2H), 4.66 (dd, J=6.0, 6.8 Hz, 2H), 4.38-4.18 (m, 1H), 3.32-3.28 (m, 2H), 2.38-2.25 (m, 6H), 1.78-1.64 (m, 2H), 1.54-1.44 (m, 4H), 1.42-1.32 (m, 2H). m/z: [ESI⁺] 475 (M+H)⁺. (C₂₇H₃₀N₄O₂S).

### (S)—N-(3-(diethylamino)propyl)-2-(4-(2,2,2-trifluoro-1-(methylamino)ethyl)phenyl)benzo[d]imidazo[2,1-b]thiazole-7-carboxamide (Compound 294)

### (R)—N-(3-(diethylamino)propyl)-2-(4-(2,2,2-trifluoro-1-(methylamino)ethyl)phenyl)benzo[d]imidazo[2,1-b]thiazole-7-carboxamide (Compound 293)

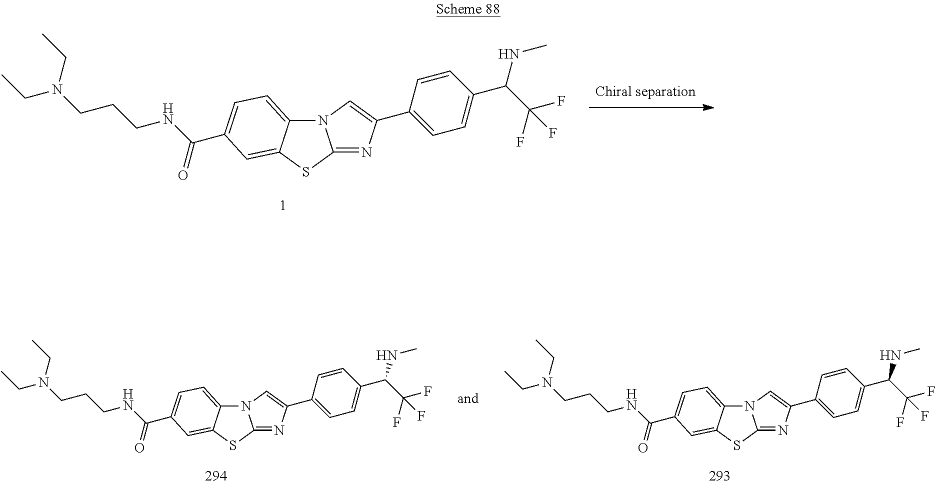 [SMILES: CCN(CC)CCCNC(=O)c1ccc2c(c1)sc1nc(-c3ccc(C(NC)C(F)(F)F)cc3)cn12 ; CCN(CC)CCCNC(=O)c1ccc2c(c1)sc1nc(-c3ccc([C@H](NC)C(F)(F)F)cc3)cn12 ; CCN(CC)CCCNC(=O)c1ccc2c(c1)sc1nc(-c3ccc([C@@H](NC)C(F)(F)F)cc3)cn12]

N-(3-(diethylamino)propyl)-2-(4-(2,2,2-trifluoro-1-(methylamino)ethyl)phenyl)benzo[d]imidazo[2,1-b]thiazole-7-carboxamide (550 mg, 1.063 mmol) was separated by chiral HPLC with the following conditions (Column: CHIRALPAK IG, 2×25 cm, 5 μm; Mobile Phase A: Hexane (0.2% diethylamine), Mobile Phase B:ethanol:dichloromethane=1:1; How rate:20 mL/min; Gradient:30 B to 30 B in 15 min; 220/254 nm; RT1:11.5; RT2:13.552). The faster eluting peak was concentrated under reduced pressure to afford (S)—N-(3-(diethylamino)propyl)-2-(4-(2,2,2-trifluoro-1-(methylamino)ethyl)phenyl)benzo[d] imidazo[2,1-b]thiazole-7-carboxamide as an off-white solid.

Yield 39 mg (7%). ¹H NMR (400 MHz, DMSO) δ 8.85 (s, 1H), 8.63 (t, J=5.6 Hz, 1H), 8.49 (d, J=1.6 Hz, 1H), 8.07 (d, J=8.4 Hz, 1H), 8.02 (dd, J=1.6, 8.4 Hz, 1H), 7.90 (d, J=8.0 Hz, 2H), 7.54 (d, J=8.0 Hz, 2H), 4.41-4.17 (m, 1H), 3.32-3.28 (m, 2H), 3.00-2.75 (m, 1H), 2.52-2.40 (m, 6H), 2.26 (d, J=5.6 Hz, 3H), 1.71-1.56 (m, 2H), 0.96 (t, J=7.2 Hz, 6H). ¹⁹F NMR (376 MHz, CDCl₃) δ −73.99. m/z: [ESI⁺] 518 (M+H)⁺. (C₂₆H₃₀F₃N₅OS).

The slower eluting peak was concentrated under reduced pressure to afford (R)—N-(3-(diethylamino)propyl)-2-(4-(2,2,2-trifluoro-1-(methylamino)ethyl)phenyl)benzo[d] imidazo[2,1-b]thiazole-7-carboxamide as an off-white solid.

Yield 39 mg (7%). ¹H NMR (400 MHz, DMSO) δ 8.85 (s, 1H), 8.63 (t, J=5.6 Hz, 1H), 8.49 (d, J=1.6 Hz, 1H), 8.07 (d, J=8.4 Hz, 1H), 8.02 (dd, J=1.6, 8.4 Hz, 1H), 7.90 (d, J=8.0 Hz, 2H), 7.54 (d, J=8.0 Hz, 2H), 4.41-4.17 (m, 1H), 3.32-3.28 (m, 2H), 3.00-2.75 (m, 1H), 2.52-2.40 (m, 6H), 2.26 (d, J=5.6 Hz, 3H), 1.71-1.56 (m, 2H), 0.96 (t, J=7.2 Hz, 6H). ¹⁹F NMR (376 MHz, CDCl₃) δ −73.99. m/z: [ESI⁺] 518 (M+H)⁺. (C₂₆H₃₀F₃N₅OS).

### 2-(4-((cyclopropylamino)methyl)-2-fluorophenyl)-N-(3-(diethylamino)propyl)benzo[d]imidazo[2,1-b]thiazole-7-carboxamide (Compound 331)

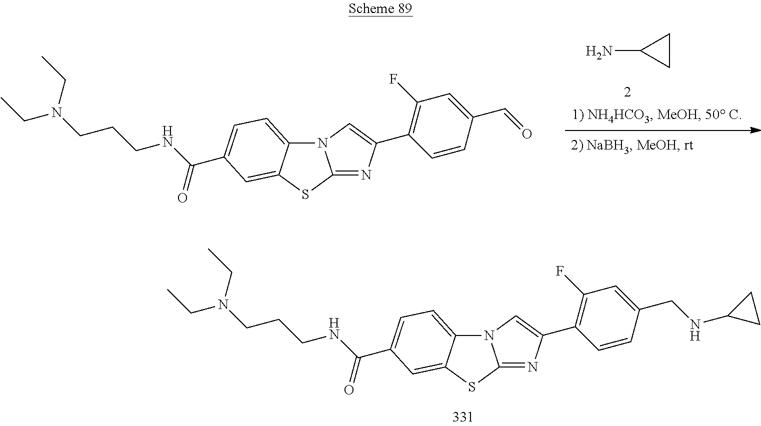

To a stirred solution of N-(3-(diethylamino)propyl)-2-(2-fluoro-4-formylphenyl)benzo[d]imidazo[2,1-b]thiazole-7-carboxamide (260 mg, 0.575 mmol) and cyclopropanamine (66 mg, 1.156 mmol) in methanol (3 mL) was added ammonium bicarbonate (91 mg, 1.151 mmol). The reaction mixture was stirred at 50° C. for 0.5 h. To the above mixture was added sodium borohydride (44 mg, 1.163 mmol) portion-wise over 5 min at room temperature. The mixture was stirred for an additional 16 h at room temperature. The resulting mixture was concentrated under reduced pressure. The residue was purified by prep HPLC with the following conditions (Column: XBridge Prep OBD C18 Column, 30×150 mm, 5 μm; Mobile Phase A: water (plus 10 mmol/L ammonium bicarbonate), Mobile Phase B: acetonitrile; Flow rate: 60 mL/min; Gradient: 23% B to 40% B in 8 min; Detector: UV 254/220 nm. The fractions contained desired product were collected, concentrated under reduced pressure and lyophilized to afford 2-(4-((cyclopropylamino)methyl)-2-fluorophenyl)-N-(3-(diethylamino)propyl)benzo[d]imidazo[2,1-b]thiazole-7-carboxamide as an off-white solid.

Yield 59 mg (21%). ¹H NMR (400 MHz, DMSO) δ 8.68 (d, J=3.6 Hz, 1H), 8.63 (t, J=5.6 Hz, 1H), 8.48 (d, J=1.6 Hz, 1H), 8.26 (d, J=8.4 Hz, 1H), 8.11-8.03 (m, 1H), 8.00 (dd, J=1.6, 8.4 Hz, 1H), 7.28 (dd, J=1.6, 12.4 Hz, 1H), 7.25 (dd, J=1.6, 8.0 Hz, 1H), 3.76 (s, 2H), 3.32-3.25 (m, 2H), 2.48-2.42 (m, 6H), 2.11-2.01 (m, 1H), 1.72-1.61 (m, 2H), 0.95 (t, J=7.2 Hz, 6H), 0.41-0.30 (m, 2H), 0.30-0.22 (m, 2H). Aliphatic NH proton not observed. ¹⁹F NMR (376 MHz, DMSO) δ −114.22. m/z: [ESI⁺] 494 (M+H)⁺. (C₂₇H₃₂FN₅OS).

### 2-(4-(aminomethyl)-2-fluorophenyl)-N-(3-(diethylamino)propyl)benzo[d]imidazo[2,1-b]thiazole-7-carboxamide diformate (Compound 298)

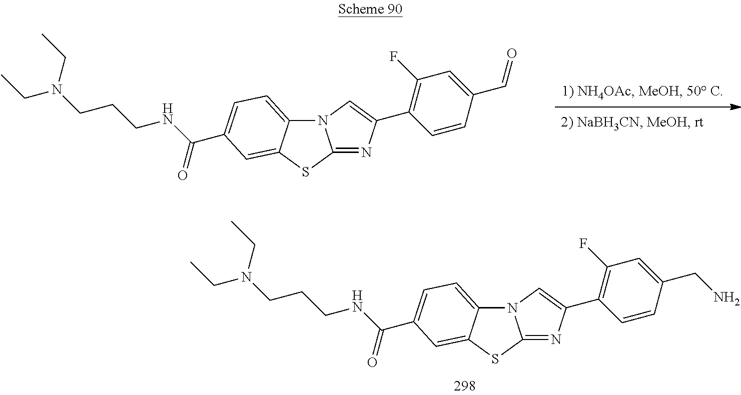 [SMILES: CCN(CC)CCCNC(=O)c1ccc2c(c1)sc1nc(-c3ccc(C=O)cc3F)cn12 ; CCN(CC)CCCNC(=O)c1ccc2c(c1)sc1nc(-c3ccc(CN)cc3F)cn12]

A mixture of N-(3-(diethylamino)propyl)-2-(2-fluoro-4-formylphenyl)benzo[d]imidazo[2,1-b]thiazole-7-carboxamide (600 mg, 1.326 mmol) and ammonium acetate (307 mg, 3.983 mmol) in methanol (20 mL) was stirred at 50° C. for 30 min. To the above mixture was added sodium cyanoborohydride (167 mg, 2.658 mmol) at room temperature. The mixture was stirred for additional 2 h at room temperature. The resulting mixture was purified by reverse flash chromatography with the following conditions: column, C18, 20-40 um, 330 g; Mobile Phase A: water (plus 10 mM formic acid); Mobile Phase B: acetonitrile; Flow rate: 80 mL/min; Gradient: 25% B-40% B in 25 min; Detector: UV 254/220 nm. The desired fractions were collected, concentrated under reduced pressure and lyophilized to afford 2-(4-(aminomethyl)-2-fluorophenyl)-N-(3-(diethylamino)propyl)benzo[d]imidazo[2,1-b]thiazole-7-carboxamide diformate as a white solid.

Yield 47 mg (7%). ¹H NMR (400 MHz, DMSO) δ 8.72 (d, J=3.6 Hz, 1H), 8.69 (t, J=5.6 Hz, 1H), 8.50 (d, J=1.6 Hz, 1H), 8.33 (s, 2H, formic acid), 8.27 (d, J=8.4 Hz, 1H), 8.18-8.12 (m, 1H), 8.02 (dd, J=1.6, 8.4 Hz, 1H), 7.44 (dd, J=1.6, 12.4 Hz, 1H), 7.35 (dd, J=1.6, 8.0 Hz, 1H), 3.99 (s, 2H), 3.38-3.29 (m, 2H), 2.65-2.52 (m, 6H), 1.78-1.67 (m, 2H), 1.01 (t, J=7.2 Hz, 6H). Aliphatic NH₂ protons not observed. ¹⁹F NMR (376 MHz, DMSO) δ −113.53. m/z: [ESI⁺] 454 (M+H)⁺. (C₂₄H₂₈FN₅OS).

### 2-(4-((cyclopropylamino)methyl)-2-fluorophenyl)-N-(3-(piperidin-1-yl)propyl)benzo[d]imidazo[2,1-b]thiazole-7-carboxamide (Compound 368)

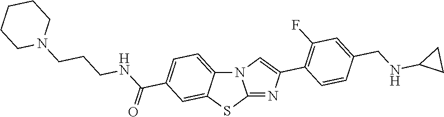 [SMILES: O=C(NCCCN1CCCCC1)c1ccc2c(c1)sc1nc(-c3ccc(CNC4CC4)cc3F)cn12]

Compound 2-(4-((cyclopropylamino)methyl)-2-fluorophenyl)-N-(3-(piperidin-1-yl)propyl)benzo[d]imidazo[2,1-b]thiazole-7-carboxamide was prepared from 2-(2-fluoro-4-formylphenyl)-N-(3-(piperidin-1-yl)propyl)benzo[d]imidazo[2,1-b]thiazole-7-carboxamide (210 mg, 0.452 mmol) and cyclopropanamine (39 mg, 0.683 mmol) following a similar procedure to that described for the synthesis of 2-(4-((cyclopropylamino)methyl)-2-fluorophenyl)-N-(3-(diethylamino)propyl)benzo[d]imidazo[2,1-b]thiazole-7-carboxamide, and was isolated as an off-white solid.

Yield 16 mg (7%). ¹H NMR (400 MHz, DMSO) δ 8.68 (d, J=3.6 Hz, 1H), 8.63 (t, J=5.6 Hz, 1H), 8.49 (d, J=1.6 Hz, 1H), 8.26 (d, J=8.4 Hz, 1H), 8.11-8.04 (m, 1H), 8.01 (dd, J=1.6, 8.4 Hz, 1H), 7.32-7.22 (m, 2H), 3.77 (s, 2H), 3.37-3.32 (m, 2H), 2.85 (br s, 1H), 2.40-2.27 (m, 6H), 2.12-2.02 (m, 1H), 1.76-1.64 (m, 2H), 1.57-1.43 (m, 4H), 1.42-1.32 (m, 2H), 0.41-0.33 (m, 2H), 0.34-0.23 (m, 2H). ¹⁹F NMR (376 MHz, DMSO) δ −114.24. m/z: [ESI⁺] 506 (M+H)⁺. (C₂₈H₃₂FN₅OS).

### N-(3-(diethylamino)propyl)-2-(4-((methylamino)methyl)phenyl)benzo[d]imidazo[2,1-b]thiazole-7-carboxamide hemi-formate (Compound 288)

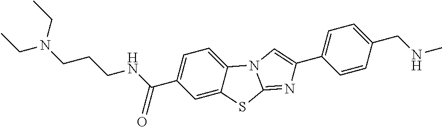 [SMILES: CCN(CC)CCCNC(=O)c1ccc2c(c1)sc1nc(-c3ccc(CNC)cc3)cn12]

Compound N-(3-(diethylamino)propyl)-2-(4-((methylamino)methyl)phenyl)benzo[d]imidazo[2,1-b]thiazole-7-carboxamide hemi-formate was prepared from N-(3-(diethylamino)propyl)-2-(4-formylphenyl)benzo[d]imidazo[2,1-b]thiazole-7-carboxamide (300 mg, 0.690 mmol) and methanamine hydrochloride (70 mg, 1.044 mmol) following a similar procedure to that described for the synthesis of tert-butyl (3-(4-fluoropiperidin-1-yl)propyl)carbamate, and was isolated as a white solid.

Yield 34 mg (9%). ¹H NMR (400 MHz, DMSO) δ 8.84 (s, 1H), 8.72 (t, J=5.6 Hz, 1H), 8.50 (d, J=1.6 Hz, 1H), 8.34 (s, 1.92H, formic acid), 8.09-8.00 (m, 2H), 7.89 (d, J=8.0 Hz, 2H), 7.51 (d, J=8.0 Hz, 2H), 3.98 (s, 2H), 3.39-3.30 (m, 2H), 2.70-2.60 (m, 6H), 2.47 (s, 3H), 1.81-1.69 (m, 2H), 1.03 (t, J=7.2 Hz, 6H). Aliphatic NH proton not observed. m/z: [ESI⁺] 450 (M+H)⁺. (C₂₅H₃₁N₅OS).

### N-(3-(diethylamino)propyl)-2-(4-(pyrrolidin-1-ylmethyl)phenyl)benzo[d]imidazo[2,1-b]thiazole-7-carboxamide hemi-formate (Compound 289)

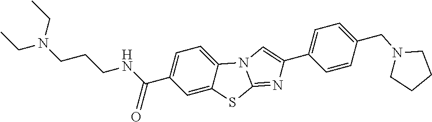 [SMILES: CCN(CC)CCCNC(=O)c1ccc2c(c1)sc1nc(-c3ccc(CN4CCCC4)cc3)cn12]

Compound N-(3-(diethylamino)propyl)-2-(4-(pyrrolidin-1-ylmethyl)phenyl)benzo[d]imidazo[2,1-b]thiazole-7-carboxamide hemi-formate was prepared from N-(3-(diethylamino)propyl)-2-(4-formylphenyl)benzo[d]imidazo[2,1-b]thiazole-7-carboxamide (200 mg, 0.460 mmol) and pyrrolidine (49 mg, 0.689 mmol) following a similar procedure to that described for the synthesis of tert-butyl (3-(4-fluoropiperidin-1-yl)propyl)carbamate, and was isolated as a white solid.

Yield 13 mg (5%). ¹H NMR (400 MHz, DMSO) δ 8.79 (s, 1H), 8.65 (d, J=5.6 Hz, 1H), 8.49 (d, J=1.6 Hz, 1H), 8.22 (s, 1.26H, formic acid), 8.12-7.98 (m, 2H), 7.82 (d, J=8.0 Hz, 2H), 7.38 (d, J=8.0 Hz, 2H), 3.63 (s, 2H), 3.35-3.30 (m, 2H), 2.64-2.56 (m, 10H), 1.78-1.66 (m, 6H), 1.04-0.96 (m, 6H). m/z: [ESI⁺] 490 (M+H)⁺. (C₂₈H₃₅N₅OS).

### 2-(4-((cyclopropylamino)methyl)phenyl)-N-(3-(diethylamino)propyl)benzo[d]imidazo[2,1-b]thiazole-7-carboxamide (Compound 300)

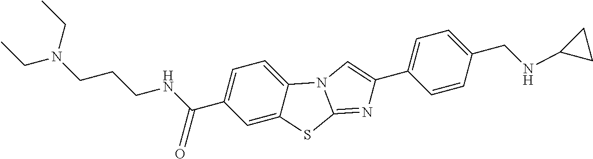 [SMILES: CCN(CC)CCCNC(=O)c1ccc2c(c1)sc1nc(-c3ccc(CNC4CC4)cc3)cn12]

Compound 2-(4-((cyclopropylamino)methyl)phenyl)-N-(3-(diethylamino)propyl)benzo[d]imidazo[2,1-b]thiazole-7-carboxamide was prepared from N-(3-(diethylamino)propyl)-2-(4-formylphenyl)benzo[d]imidazo[2,1-b]thiazole-7-carboxamide (200 mg, 0.460 mmol) and cyclopropanamine (39 mg, 0.683 mmol) following a similar procedure to that described for the synthesis of tert-butyl (3-(4-fluoropiperidin-1-yl)propyl)carbamate, and was isolated as a yellow solid.

Yield 82 mg (37%). ¹H NMR (400 MHz, DMSO) δ 8.78 (s, 1H), 8.63 (t, J=5.6 Hz, 1H), 8.48 (d, J=1.6 Hz, 1H), 8.09-7.98 (m, 2H), 7.81 (d, J=8.0 Hz, 2H), 7.39 (d, J=8.0 Hz, 2H), 3.75 (s, 2H), 3.33-3.28 (m, 2H), 2.73 (br s, 1H), 2.49-2.41 (m, 6H), 2.12-2.02 (m, 1H), 1.73-1.61 (m, 2H), 0.96 (t, J=7.2 Hz, 6H), 0.41-0.33 (m, 2H), 0.32-0.23 (m, 2H). m/z: [ESI⁺] 476 (M+H)⁺. (C₂₇H₃₃N₅OS).

### N-(3-(diethylamino)propyl)-2-(4-(((2-methoxyethyl)amino)methyl)phenyl)benzo[d]imidazo[2,1-b]thiazole-7-carboxamide hemi-formate (Compound 292)

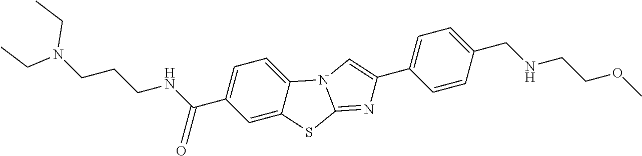 [SMILES: CCN(CC)CCCNC(=O)c1ccc2c(c1)sc1nc(-c3ccc(CNCCOC)cc3)cn12]

Compound N-(3-(diethylamino)propyl)-2-(4-(((2-methoxyethyl)amino)methyl)phenyl)benzo[d]imidazo[2,1-b]thiazole-7-carboxamide hemi-formate was prepared from N-(3-(diethylamino)propyl)-2-(4-formylphenyl)benzo[d]imidazo[2,1-b]thiazole-7-carboxamide (300 mg, 0.690 mmol) and 2-methoxyethan-1-amine (78 mg, 1.038 mmol) following a similar procedure to that described for the synthesis of tert-butyl (3-(4-fluoropiperidin-1-yl)propyl)carbamate, and was isolated as a yellow solid.

Yield 129 mg (32%). ¹H NMR (400 MHz, DMSO) δ 8.81 (s, 1H), 8.70 (t, J=5.6 Hz, 1H), 8.50 (d, J=1.6 Hz, 1H), 8.27 (s, 2.13H, formic acid), 8.09-8.00 (m, 2H), 7.83 (d, J=8.0 Hz, 2H), 7.44 (d, J=8.0 Hz, 2H), 3.84 (s, 2H), 3.47 (t, J=5.6 Hz, 2H), 3.37-3.31 (m, 2H), 3.26 (s, 3H), 2.77 (t, J=5.6 Hz, 2H), 2.72-2.62 (m, 6H), 1.82-1.70 (m, 2H), 1.03 (t, J=7.2 Hz, 6H). Aliphatic NH proton not observed. m/z: [ESI⁺] 494 (M+H)⁺. (C₂₇H₃₅N₅O₂S).

### 2-(4-(methylcarbamoyl)phenyl)-N-(3-(piperidin-1-yl)propyl)imidazo[2′,1′:2,3]thiazolo[4,5-c]pyridine-7-carboxamide (Compound 406)

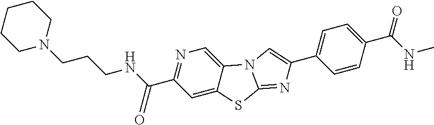 [SMILES: CNC(=O)c1ccc(-c2cn3c(n2)sc2cc(C(=O)NCCCN4CCCCC4)ncc23)cc1]

Compound 2-(4-(methylcarbamoyl)phenyl)-N-(3-(piperidin-1-yl)propyl)imidazo[2′,1′:2,3]thiazolo[4,5-c]pyridine-7-carboxamide was prepared from 4-(7-chloroimidazo[2′,1′:2,3]thiazolo[4,5-c]pyridin-2-yl)-N-methylbenzamide (2.00 g, 5.83 mmol) and 3-(piperidin-1-yl)propan-1-amine (3.32 g, 23.34 mmol) following a similar procedure to that described for the synthesis of tert-butyl (4-(7-((3-(piperidin-1-yl)propyl)carbamoyl)benzo[d]imidazo[2,1-b]thiazol-2-yl)-3-(trifluoromethyl)benzyl)carbamate, and was isolated as a white solid.

Yield 22 mg (1%). ¹H NMR (400 MHz, DMSO) δ 9.35 (t, J=5.6 Hz, 1H), 9.24 (s, 1H), 9.07 (s, 1H), 8.83 (s, 1H), 8.48 (q, J=4.4 Hz, 1H), 7.98-7.90 (m, 4H), 3.44-3.37 (m, 2H), 2.81 (d, J=4.4 Hz, 3H), 2.43-2.28 (m, 6H), 1.76-1.67 (m, 2H), 1.64-1.54 (m, 4H), 1.49-1.40 (m, 2H). m/z: [ESI⁺] 477 (M+H)⁺. (C₂₅H₂₈N₆O₂S).

### N-(3-(ethylamino)propyl)-2-(2-fluoro-4-(methylcarbamoyl)phenyl)benzo[d]imidazo[2,1-b]thiazole-7-carboxamide formate (Compound 362)

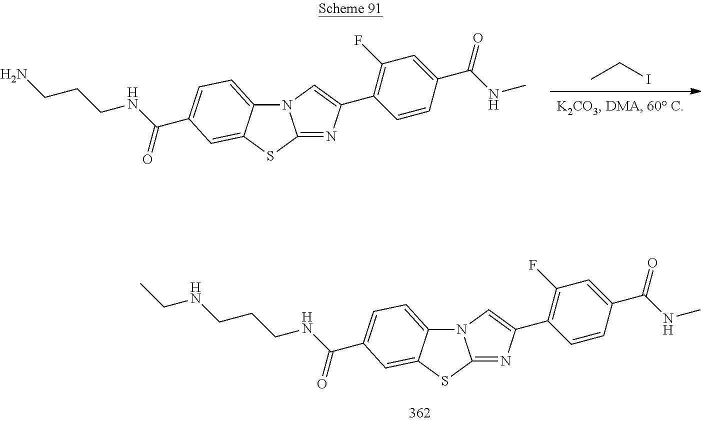

To a stirred solution of N-(3-aminopropyl)-2-(2-fluoro-4-(methylcarbamoyl)phenyl)benzo[d]imidazo[2,1-b]thiazole-7-carboxamide formate (650 mg, 1.379 mmol) and ethyl iodide (274 mg, 1.767 mmol) in N,N-dimethylacetamide (20 mL) was added potassium carbonate (486 mg, 3.517 mmol) portion-wise at room temperature. The resulting mixture was stirred for 2 h at 60° C. under a nitrogen atmosphere. The mixture was allowed to cool to room temperature and was purified by reverse phase flash chromatography with the following conditions: Column: WelHash™ C18-I, 20-40 m, 330 g; Eluent A: water (plus 10 mmol/L formic acid); Eluent B: acetonitrile; Gradient: 40%-60% B in 25 min; Flow rate: 80 mL/min; Detector: UV 220/254 nm. The fractions containing the desired product were collected, concentrated under reduced pressure and lyophilized to afford N-(3-(ethylamino)propyl)-2-(2-fluoro-4-(methylcarbamoyl)phenyl)benzo[d]imidazo[2,1-b]thiazole-7-carboxamide formate as a light yellow solid.

Yield 80 mg (12%). ¹H NMR (400 MHz, DMSO) δ 8.86-8.78 (m, 2H), 8.59-8.56 (m, 1H), 8.53 (d, J=1.6 Hz, 1H), 8.37 (s, 1H, formic acid), 8.30 (d, J=8.4 Hz, 1H), 8.24 (t, J=8.0 Hz, 1H), 8.04 (dd, J=1.6, 8.4 Hz, 1H), 7.83-7.75 (m, 2H), 3.40-3.35 (m, 2H), 2.87 (d, J=4.4 Hz, 3H), 2.86-2.71 (m, 4H), 1.82-1.76 (m, 2H), 1.11 (t, J=7.2 Hz, 3H). Aliphatic NH proton not observed. ¹⁹F NMR (376 MHz, DMSO) δ −113.10. m/z: [ESI⁺] 454 (M+H)⁺. (C₂₃H₂₄FN₅O₂S).

### 2-(2-(2-(3-(but-3-yn-1-yl)-3H-diazirin-3-yl)ethoxy)phenyl)-N-(3-(piperidin-1-yl)propyl)benzo[d]imidazo[2,1-b]thiazole-7-carboxamide hemi-formate (Compound 326)

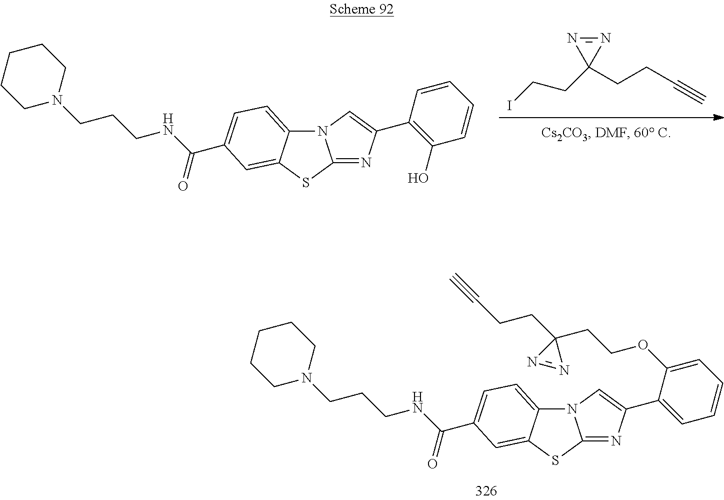

To a stirred solution of 2-(2-hydroxyphenyl)-N-(3-(piperidin-1-yl)propyl)benzo[d]imidazo[2,1-b]thiazole-7-carboxamide (100 mg, 0.230 mmol) and cesium carbonate (224 mg, 0.687 mmol) in N,N-dimethylformamide (2 mL) was added 3-(but-3-yn-1-yl)-3-(2-iodoethyl)-3H-diazirine (228 mg, 0.919 mmol) dropwise at room temperature. The mixture was stirred at 60° C. for 3 h. The resulting mixture was purified by reverse phase flash chromatography with the following conditions: Column: Spherical C18, 20-40 um, 330 g; Mobile Phase A: water (plus 10 mM formic acid); Mobile Phase B: acetonitrile; Flow rate: 40 mL/min; Gradient: 50% B-70% B in 20 min; Detector: UV 254/215 nm. The fractions containing desired product were collected at 60% B and concentrated under reduced pressure to afford 2-(2-(2-(3-(but-3-yn-1-yl)-3H-diazirin-3-yl)ethoxy)phenyl)-N-(3-(piperidin-1-yl)propyl)benzo[d]imidazo[2,1-b]thiazole-7-carboxamide hemi-formate as an off-white solid.

Yield 57 mg (44%). ¹H NMR (400 MHz, DMSO) δ 8.71 (s, 1H), 8.67 (t, J=5.6 Hz, 1H), 8.49 (d, J=1.6 Hz, 1H), 8.31 (s, 0.21H, formic acid), 8.19 (dd, J=1.6, 7.6 Hz, 1H), 8.13 (d, J=8.4 Hz, 1H), 8.04 (dd, J=1.6, 8.4 Hz, 1H), 7.34-7.22 (m, 1H), 7.11 (d, J=8.4 Hz, 1H), 7.06 (dd, J=7.6, 8.4 Hz, 1H), 4.07 (t, J=6.0 Hz, 2H), 3.38-3.28 (m, 2H), 2.76 (t, J=2.4 Hz, 1H), 2.48-2.36 (m, 6H), 2.13 (t, J=6.0 Hz, 2H), 2.06 (dt, J=2.4, 7.2 Hz, 2H), 1.79-1.67 (m, 4H), 1.59-1.46 (m, 4H), 1.45-1.33 (m, 2H). m/z: [ESI⁺]555 (M+H)⁺. (C₃₁H₃₄N₆O₂S).

### (R)—N-((1-(2-(3-(but-3-yn-1-yl)-3H-diazirin-3-yl)ethyl)pyrrolidin-3-yl)methyl)-2-(p-tolyl)benzo[d]imidazo[2,1-b]thiazole-7-carboxamide (Compound 324)

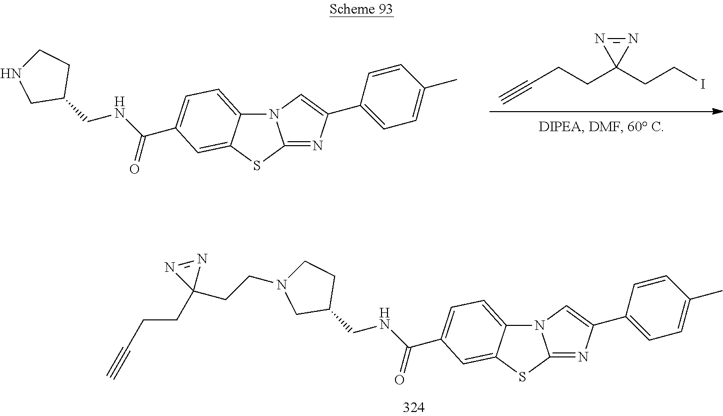

A solution of (S)—N-(pyrrolidin-3-ylmethyl)-2-(p-tolyl)benzo[d]imidazo[2,1-b]thiazole-7-carboxamide hydrochloride (143 mg, 0.335 mmol), 3-(but-3-yn-1-yl)-3-(2-iodoethyl)-3H-diazirine (70 mg, 0.282 mmol) and N-ethyl-N-isopropylpropan-2-amine (109 mg, 0.843 mmol) in N,N-dimethylformamide (5 mL) was stirred in the dark for 16 h at 60° C. under a nitrogen atmosphere. The resulting solution was cooled to room temperature and purified by reverse flash chromatography with the following conditions: column, C18 silica gel; Mobile Phase A: water (plus 5 mM ammonium bicarbonate); Mobile Phase B: acetonitrile; Flow rate: 40 mL/min; Gradient: 70% B-85% B in 20 min; Detector: UV 254/215 nm. The fractions containing desired product were collected, concentrated under reduced pressure and lyophilized to afford (R)—N-((1-(2-(3-(but-3-yn-1-yl)-3H-diazirin-3-yl)ethyl)pyrrolidin-3-yl)methyl)-2-(p-tolyl)benzo[d]imidazo[2,1-b]thiazole-7-carboxamide as an off-white solid.

Yield 68 mg (40%). ¹H NMR (300 MHz, DMSO) δ 8.75 (s, 1H), 8.60 (t, J=5.7 Hz, 1H), 8.46 (d, J=1.2 Hz, 1H), 8.06-7.97 (m, 2H), 7.76 (d, J=8.1 Hz, 2H), 7.25 (d, J=8.1 Hz, 2H), 3.31-3.20 (m, 3H), 2.81 (t, J=2.7 Hz, 1H), 2.43-2.24 (m, 7H), 2.18 (t, J=7.5 Hz, 2H), 2.00 (dt, J=2.7, 7.5 Hz, 2H), 1.93-1.79 (m, 1H), 1.62-1.51 (m, 4H), 1.52-1.39 (m, 1H). m/z: [ESI⁺]511 (M+H)⁺. (C₂₉H₃₀N₆OS).

### (S)—N-((1-(2-(3-(but-3-yn-1-yl)-3H-diazirin-3-yl)ethyl)pyrrolidin-3-yl)methyl)-2-(p-tolyl)benzo[d]imidazo[2,1-b]thiazole-7-carboxamide (Compound 325)

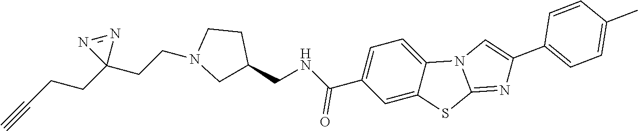 [SMILES: C#CCCC1(CCN2CC[C@@H](CNC(=O)c3ccc4c(c3)sc3nc(-c5ccc(C)cc5)cn34)C2)N=N1]

Compound (S)—N-((1-(2-(3-(but-3-yn-1-yl)-3H-diazirin-3-yl)ethyl)pyrrolidin-3-yl)methyl)-2-(p-tolyl)benzo[d]imidazo[2,1-b]thiazole-7-carboxamide was prepared from (R)—N-(pyrrolidin-3-ylmethyl)-2-(p-tolyl)benzo[d]imidazo[2,1-b]thiazole-7-carboxamide hydrochloride (143 mg, 0.335 mmol) and 3-(but-3-yn-1-yl)-3-(2-iodoethyl)-3H-diazirine (99 mg, 0.399 mmol) following a similar procedure to that described for the synthesis of (R)—N-((1-(2-(3-(but-3-yn-1-yl)-3H-diazirin-3-yl)ethyl)pyrrolidin-3-yl)methyl)-2-(p-tolyl)benzo[d]imidazo[2,1-b]thiazole-7-carboxamide, and was isolated as an off-white solid.

Yield 75 mg (44%). ¹H NMR (300 MHz, DMSO) δ 8.75 (s, 1H), 8.60 (t, J=5.7 Hz, 1H), 8.46 (d, J=1.2 Hz, 1H), 8.06-7.97 (m, 2H), 7.76 (d, J=8.1 Hz, 2H), 7.25 (d, J=8.1 Hz, 2H), 3.31-3.20 (m, 3H), 2.81 (t, J=2.7 Hz, 1H), 2.43-2.24 (m, 7H), 2.18 (t, J=7.5 Hz, 2H), 2.00 (dt, J=2.7, 7.5 Hz, 2H), 1.93-1.79 (m, 1H), 1.62-1.51 (m, 4H), 1.52-1.39 (m, 1H). m/z: [ESI⁺] 511 (M+H)⁺. (C₂₉H₃₀N₆OS).

### N-(3-((2-(3-(but-3-yn-1-yl)-3H-diazirin-3-yl)ethyl)(ethyl)amino)propyl)-2-(4-(methylcarbamoyl)phenyl)benzo[d]imidazo[2,1-b]thiazole-7-carboxamide hemi-formate (Compound 323)

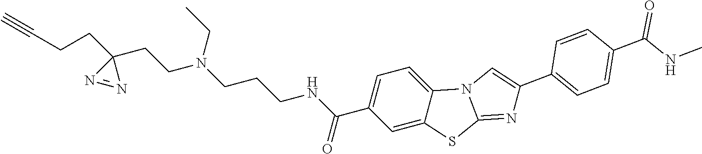 [SMILES: C#CCCC1(CCN(CC)CCCNC(=O)c2ccc3c(c2)sc2nc(-c4ccc(C(=O)NC)cc4)cn23)N=N1]

Compound N-(3-((2-(3-(but-3-yn-1-yl)-3H-diazirin-3-yl)ethyl)(ethyl)amino)propyl)-2-(4-(methylcarbamoyl)phenyl)benzo[d]imidazo[2,1-b]thiazole-7-carboxamide hemi-formate was prepared from N-(3-(ethylamino)propyl)-2-(4-(methylcarbamoyl)phenyl)benzo[d]imidazo[2,1-b]thiazole-7-carboxamide 2,2,2-trifluoroacetate (150 mg, 0.273 mmol) and 3-(but-3-yn-1-yl)-3-(2-iodoethyl)-3H-diazirine (76 mg, 0.306 mmol) following a similar procedure to that described for the synthesis of (R)—N-((1-(2-(3-(but-3-yn-1-yl)-3H-diazirin-3-yl)ethyl)pyrrolidin-3-yl)methyl)-2-(p-tolyl)benzo[d]imidazo[2,1-b]thiazole-7-carboxamide, and was isolated as a light yellow solid.

Yield 72 mg (46%). ¹H NMR (300 MHz, DMSO) δ 8.91 (s, 1H), 8.56 (t, J=5.7 Hz, 1H), 8.48 (s, 1H), 8.44 (q, J=4.5 Hz, 1H), 8.27 (s, 0.41H, formic acid), 8.09-7.98 (m, 2H), 7.98-7.87 (m, 4H), 3.37-3.25 (m, 2H), 2.80 (d, J=4.5 Hz, 3H), 2.79 (t, J=2.7 Hz, 1H), 2.47-2.35 (m, 4H), 2.22 (t, J=7.2 Hz, 2H), 2.00 (dt, J=2.7, 7.2 Hz, 2H), 1.71-1.44 (m, 6H), 0.92 (t, J=7.2 Hz, 3H). m/z: [ESI⁺]556 (M+H)⁺. (C₃₀H₃₃N₇O₂S).

### 2-(2-fluoro-4-(methylcarbamoyl)phenyl)-N-(3-(4-fluoropiperidin-1-yl)propyl)benzo[d]imidazo[2,1-b]thiazole-7-carboxamide (Compound 320)

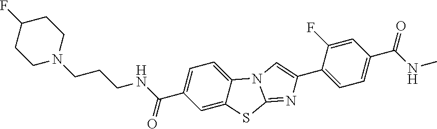 [SMILES: CNC(=O)c1ccc(-c2cn3c(n2)sc2cc(C(=O)NCCCN4CCC(F)CC4)ccc23)c(F)c1]

Compound 2-(2-fluoro-4-(methylcarbamoyl)phenyl)-N-(3-(4-fluoropiperidin-1-yl)propyl)benzo[d]imidazo[2,1-b]thiazole-7-carboxamide was prepared from 2-(2-fluoro-4-(methylcarbamoyl)phenyl)benzo[d]imidazo[2,1-b]thiazole-7-carboxylic acid (400 mg, 1.083 mmol) and 3-(4-fluoropiperidin-1-yl)propan-1-amine dihydrochloride (303 mg, 1.300 mmol) following a similar procedure to that described for the synthesis of 2-bromo-N-(3-(piperidin-1-yl)propyl)benzo[d]imidazo[2,1-b]thiazole-7-carboxamide, and was isolated as a dark yellow solid.

Yield 18 mg (3%). ¹H NMR (400 MHz, DMSO) δ 8.83 (d, J=3.6 Hz, 1H), 8.61 (t, J=5.6 Hz, 1H), 8.56 (q, J=4.4 Hz, 1H), 8.50 (d, J=1.6 Hz, 1H), 8.30 (d, J=8.4 Hz, 1H), 8.24 (t, J=8.0 Hz, 1H), 8.02 (dd, J=1.6, 8.4 Hz, 1H), 7.84-7.74 (m, 2H), 4.76-4.70 (m, 0.5H), 4.64-4.55 (m, 0.5H), 3.36-3.34 (m, 2H), 2.82 (d, J=4.4 Hz, 3H), 2.69-2.65 (m, 2H), 2.41-2.32 (m, 2H), 2.32-2.26 (m, 2H), 1.95-1.77 (m, 2H), 1.74-1.66 (m, 4H). ¹⁹F NMR (376 MHz, DMSO) δ −113.08. Fluorine signal of 4-fluoropiperidine not observed. m/z: [ESI⁺] 512 (M+H)⁺. (C₂₆H₂₇F₂N₅O₂S).

### 2-(2-fluoro-4-(methylcarbamoyl)phenyl)-N-(3-(pyrrolidin-1-yl)propyl)benzo[d]imidazo[2,1-b]thiazole-7-carboxamide (Compound 321)

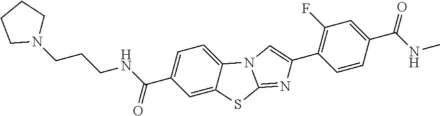 [SMILES: CNC(=O)c1ccc(-c2cn3c(n2)sc2cc(C(=O)NCCCN4CCCC4)ccc23)c(F)c1]

Compound 2-(2-fluoro-4-(methylcarbamoyl)phenyl)-N-(3-(pyrrolidin-1-yl)propyl)benzo[d]imidazo[2,1-b]thiazole-7-carboxamide was prepared from 2-(2-fluoro-4-(methylcarbamoyl)phenyl)benzo[d]imidazo[2,1-b]thiazole-7-carboxylic acid (400 mg, 1.083 mmol) and 3-(pyrrolidin-1-yl)propan-1-amine (167 mg, 1.302 mmol) following a similar procedure to that described for the synthesis of 2-bromo-N-(3-(piperidin-1-yl)propyl)benzo[d]imidazo[2,1-b]thiazole-7-carboxamide, and was isolated as an off-white solid.

Yield 13 mg (3%). ¹H NMR (400 MHz, DMSO) δ 8.82 (d, J=3.6 Hz, 1H), 8.69 (t, J=5.6 Hz, 1H), 8.57 (q, J=4.4 Hz, 1H), 8.49 (d, J=1.6 Hz, 1H), 8.29 (d, J=8.4 Hz, 1H), 8.23 (t, J=8.0 Hz, 1H), 8.01 (dd, J=1.6, 8.4 Hz, 1H), 7.82-7.74 (m, 2H), 3.38-3.31 (m, 2H), 2.81 (d, J=4.4 Hz, 3H), 2.49-2.39 (m, 6H), 1.77-1.64 (m, 6H). ¹⁹F NMR (376 MHz, DMSO) δ −113.08. m/z: [ESI⁺] 480 (M+H)⁺. (C₂₅H₂₆FN₅O₂S).

### N-(3-(diethylamino)propyl)-2-(4-((1-methylpyrrolidin-3-yl)carbamoyl)phenyl)benzo[d]imidazo[2,1-b]thiazole-7-carboxamide di-formate (Compound 286)

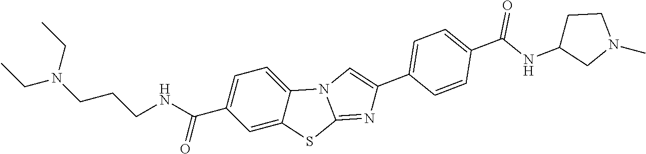 [SMILES: CCN(CC)CCCNC(=O)c1ccc2c(c1)sc1nc(-c3ccc(C(=O)NC4CCN(C)C4)cc3)cn12]

Compound N-(3-(diethylamino)propyl)-2-(4-((1-methylpyrrolidin-3-yl)carbamoyl)phenyl)benzo[d]imidazo[2,1-b]thiazole-7-carboxamide di-formate was prepared from 4-(7-((3-(diethylamino)propyl)carbamoyl)benzo[d]imidazo[2,1-b]thiazol-2-yl)benzoic acid (200 mg, 0.444 mmol) and 1-methylpyrrolidin-3-amine (45 mg, 0.449 mmol) following a similar procedure to that described for the synthesis of 2-bromo-N-(3-(piperidin-1-yl)propyl)benzo[d]imidazo[2,1-b]thiazole-7-carboxamide, and was isolated as a dark yellow solid.

Yield 63 mg (23%). ¹H NMR (400 MHz, DMSO) δ 8.94 (s, 1H), 8.69 (t, J=5.6 Hz, 1H), 8.56 (d, J=7.2 Hz, 1H), 8.51 (d, J=1.6 Hz, 1H), 8.24-8.22 (m, 2H, formic acid), 8.10-8.01 (m, 2H), 7.98-7.93 (m, 4H), 4.50-4.39 (m, 1H), 3.37-3.32 (m, 2H), 2.87-2.83 (m, 1H), 2.79-2.63 (m, 7H), 2.62-2.54 (m, 2H), 2.34 (s, 3H), 2.27-2.15 (m, 1H), 1.90-1.80 (m, 1H), 1.77-1.71 (m, 2H), 1.03 (t, J=7.2 Hz, 6H). m/z: [ESI⁺] 533 (M+H)⁺. (C₂₉H₃₆N₆O₂S).

### (S)-2-(4-(methylcarbamoyl)phenyl)-N-(2-(1-methylpyrrolidin-2-yl)ethyl)benzo[d]imidazo[2,1-b]thiazole-7-carboxamide (Compound 303)

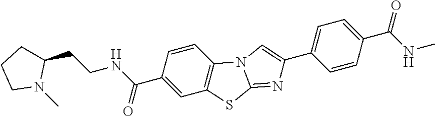 [SMILES: CNC(=O)c1ccc(-c2cn3c(n2)sc2cc(C(=O)NCC[C@@H]4CCCN4C)ccc23)cc1]

Compound (S)-2-(4-(methylcarbamoyl)phenyl)-N-(2-(1-methylpyrrolidin-2-yl)ethyl)benzo[d]imidazo[2,1-b]thiazole-7-carboxamide was prepared from 2-(4-(methylcarbamoyl)phenyl)benzo[d]imidazo[2,1-b]thiazole-7-carboxylic acid (500 mg, 1.423 mmol) and (S)-2-(1-methylpyrrolidin-2-yl)ethan-1-amine di-hydrochloride (343 mg, 1.705 mmol) following a similar procedure to that described for the synthesis of 2-bromo-N-(3-(piperidin-1-yl)propyl)benzo[d]imidazo[2,1-b]thiazole-7-carboxamide, and was isolated as a white solid.

Yield 71 mg (11%). ¹H NMR (400 MHz, DMSO) δ 8.93 (s, 1H), 8.62 (t, J=5.6 Hz, 1H), 8.49 (d, J=1.6 Hz, 1H), 8.45 (q, J=4.4 Hz, 1H), 8.08-8.01 (m, 2H), 7.97-7.89 (m, 4H), 3.36-3.30 (m, 2H), 2.97-2.92 (m, 1H), 2.81 (d, J=4.4 Hz, 3H), 2.22 (s, 3H), 2.13-2.01 (m, 2H), 2.01-1.85 (m, 2H), 1.70-1.58 (m, 2H), 1.51-1.41 (m, 2H). m/z: [ESI⁺] 462 (M+H)⁺. (C₂₅H₂₇N₅O₂S).

### N-(3-(4-fluoropiperidin-1-yl)propyl)-2-(4-(methylcarbamoyl)phenyl)benzo[d]imidazo[2,1-b]thiazole-7-carboxamide (Compound 309)

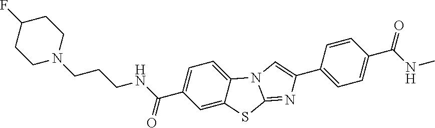 [SMILES: CNC(=O)c1ccc(-c2cn3c(n2)sc2cc(C(=O)NCCCN4CCC(F)CC4)ccc23)cc1]

Compound N-(3-(4-fluoropiperidin-1-yl)propyl)-2-(4-(methylcarbamoyl)phenyl)benzo[d]imidazo[2,1-b]thiazole-7-carboxamide was prepared from 2-(4-(methylcarbamoyl)phenyl)benzo[d]imidazo[2,1-b]thiazole-7-carboxylic acid (300 mg, 0.854 mmol) and 3-(4-fluoropiperidin-1-yl)propan-1-amine dihydrochloride (239 mg, 1.025 mmol) following a similar procedure to that described for the synthesis of 2-bromo-N-(3-(piperidin-1-yl)propyl)benzo[d]imidazo[2,1-b]thiazole-7-carboxamide, and was isolated as an off-white solid.

Yield 39 mg (9%). ¹H NMR (400 MHz, DMSO) δ 8.94 (s, 1H), 8.62 (t, J=5.6 Hz, 1H), 8.50 (d, J=1.6 Hz, 1H), 8.46 (q, J=4.4 Hz, 1H), 8.09-8.01 (m, 2H), 7.98-7.89 (m, 4H), 4.78-4.70 (m, 0.5H), 4.65-4.56 (m, 0.5H), 3.33-3.29 (m, 2H), 2.81 (d, J=4.4 Hz, 3H), 2.57-2.52 (m, 2H), 2.39-2.33 (m, 2H), 2.31-2.25 (m, 2H), 1.93-1.78 (m, 2H), 1.75-1.63 (m, 4H). Fluorine signal of 4-fluoropiperidine not observed. m/z: [ESI⁺] 494 (M+H)⁺. (C₂₆H₂₈FN₅O₂S).

### (R)-2-(4-(methylcarbamoyl)phenyl)-N-(1-methylpiperidin-3-yl)benzo[d]imidazo[2,1-b]thiazole-7-carboxamide (Compound 301)

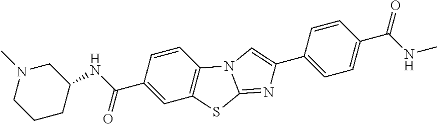

Compound (R)-2-(4-(methylcarbamoyl)phenyl)-N-(1-methylpiperidin-3-yl)benzo[d]imidazo[2,1-b]thiazole-7-carboxamide was prepared from 2-(4-(methylcarbamoyl)phenyl)benzo[d]imidazo[2,1-b]thiazole-7-carboxylic acid (300 mg, 0.854 mmol) and (R)-1-methylpiperidin-3-amine (120 mg, 1.05 mmol) following a similar procedure to that described for the synthesis of 2-bromo-N-(3-(piperidin-1-yl)propyl)benzo[d]imidazo[2,1-b]thiazole-7-carboxamide, and was isolated as an off-white solid.

Yield 120 mg (31%). ¹H NMR (400 MHz, DMSO) δ 8.94 (s, 1H), 8.53-8.51 (m, 1H), 8.47-8.43 (q, J=4.4 Hz, 1H), 8.32 (d, J=8.0 Hz, 1H), 8.07-8.04 (m, 2H), 7.98-7.89 (m, 4H), 4.01-3.94 (m, 1H), 2.91-2.83 (m, 1H), 2.81 (d, J=4.4 Hz, 3H), 2.71-2.64 (m, 1H), 2.22 (s, 3H), 1.97-1.88 (m, 2H), 1.84-1.79 (m, 1H), 1.75-1.70 (m, 1H), 1.60-1.51 (m, 1H), 1.42-1.28 (m, 1H). m/z: [ESI⁺] 448 (M+H)⁺. (C₂₄H₂₅N₅O₂S).

### 2-(4-(methylcarbamoyl)phenyl)-N-(3-methylcyclobutyl)benzo[d]imidazo[2,1-b]thiazole-7-carboxamide (Compound 280)

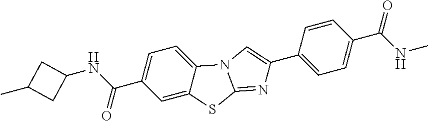

Compound 2-(4-(methylcarbamoyl)phenyl)-N-(3-methylcyclobutyl)benzo[d]imidazo[2,1-b]thiazole-7-carboxamide was prepared from 2-(4-(methylcarbamoyl)phenyl)benzo[d]imidazo[2,1-b]thiazole-7-carboxylic acid (150 mg, 0.427 mmol) and 3-methylcyclobutan-1-amine (44 mg, 0.517 mmol) following a similar procedure to that described for the synthesis of 2-bromo-N-(3-(piperidin-1-yl)propyl)benzo[d]imidazo[2,1-b]thiazole-7-carboxamide, and was isolated as an off-white solid.

Yield 15 mg (8%). ¹H NMR (400 MHz, DMSO) δ 8.94 (s, 1H), 8.75 (d, J=7.2 Hz, 0.6H), 8.68 (d, J=7.2 Hz, 0.4H), 8.52-8.49 (m, 1H), 8.45 (q, J=4.4 Hz, 1H), 8.06-8.04 (m, 2H), 7.98-7.88 (m, 4H), 4.63-4.52 (m, 0.6H), 4.32-4.23 (m, 0.4H), 2.81 (d, J=4.4 Hz, 3H), 2.48-2.39 (m, 1H), 2.38-2.33 (m, 0.6H), 2.32-2.19 (m, 1H), 2.23-2.00 (m, 0.4H), 2.00-1.88 (m, 1H), 1.73-1.65 (m, 1H), 1.17 (d, J=6.8 Hz, 1.8H), 1.09 (d, J=6.8 Hz, 1.2H). m/z: [ESI⁺] 419 (M+H)⁺. (C₂₃H₂₂N₄O₂S).

### 4-(7-(4-(diethylamino)butanamido)benzo[d]imidazo[2,1-b]thiazol-2-yl)-3-fluoro-N-methylbenzamide (Compound 365)

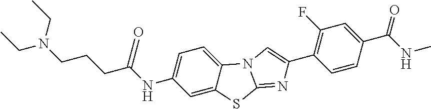 [SMILES: CCN(CC)CCCC(=O)Nc1ccc2c(c1)sc1nc(-c3ccc(C(=O)NC)cc3F)cn12]

Compound 4-(7-(4-(diethylamino)butanamido)benzo[d]imidazo[2,1-b]thiazol-2-yl)-3-fluoro-N-methylbenzamide was prepared from 4-(7-aminobenzo[d]imidazo[2,1-b]thiazol-2-yl)-3-fluoro-N-methylbenzamide (300 mg, 0.881 mmol) and 4-(diethylamino)butanoic acid (280 mg, 1.758 mmol) following a similar procedure to that described for the synthesis of 2-bromo-N-(3-(piperidin-1-yl)propyl)benzo[d]imidazo[2,1-b]thiazole-7-carboxamide, and was isolated as an off-white solid.

Yield 17 mg (4%). ¹H NMR (400 MHz, DMSO) δ 10.22 (br s, 1H), 8.71 (d, J=3.6 Hz, 1H), 8.56 (q, J=4.4 Hz, 1H), 8.35 (d, J=2.0 Hz, 1H), 8.22 (t, J=8.0 Hz, 1H), 8.12 (d, J=8.4 Hz, 1H), 7.82-7.73 (m, 2H), 7.64 (dd, J=2.0, 8.4, 1H), 2.81 (d, J=4.4 Hz, 3H), 2.48-2.36 (m, 8H), 1.77-1.69 (m, 2H), 0.96 (t, J=7.2 Hz, 6H). ¹⁹F NMR (376 MHz, DMSO) δ −113.30. m/z: [ESI⁺] 482 (M+H)⁺. (C₂₅H₂₈FN₅O₂S).

### N-methyl-4-(7-(4-(piperidin-1-yl)butanamido)benzo[d]imidazo[2,1-b]thiazol-2-yl)benzamide (Compound 312)

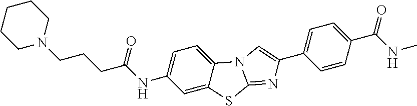 [SMILES: CNC(=O)c1ccc(-c2cn3c(n2)sc2cc(NC(=O)CCCN4CCCCC4)ccc23)cc1]

Compound N-methyl-4-(7-(4-(piperidin-1-yl)butanamido)benzo[d]imidazo[2,1-b]thiazol-2-yl)benzamide was prepared from 4-(7-aminobenzo[d]imidazo[2,1-b]thiazol-2-yl)-N-methylbenzamide (100 mg, 0.310 mmol) and 4-(piperidin-1-yl)butanoic acid hydrochloride (77 mg, 0.371 mmol) following a similar procedure to that described for the synthesis of 2-bromo-N-(3-(piperidin-1-yl)propyl)benzo[d]imidazo[2,1-b]thiazole-7-carboxamide, and was isolated as a white solid.

Yield 41 mg (28%). ¹H NMR (400 MHz, DMSO) δ 10.18 (br s, 1H), 8.82 (s, 1H), 8.44 (q, J=4.4 Hz, 1H), 8.34 (d, J=2.0 Hz, 1H), 7.99-7.88 (m, 5H), 7.66 (dd, J=2.0, 8.4, 1H), 2.80 (d, J=4.4 Hz, 3H), 2.37 (t, J=7.2 Hz, 2H), 2.31-2.25 (m, 6H), 1.80-1.72 (m, 2H), 1.53-1.44 (m, 4H), 1.41-1.32 (m, 2H). m/z: [ESI⁺] 476 (M+H)⁺. (C₂₆H₂₉N₅O₂S).

### 2-(2-fluoro-4-(methylcarbamoyl)phenyl)-N-(3-(piperidin-1-yl)propyl)benzo[4,5]thiazolo[3,2-b][1,2,4]triazole-6-carboxamide formate (Compound 386)

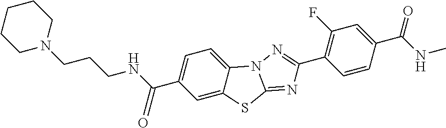 [SMILES: CNC(=O)c1ccc(-c2nc3sc4cc(C(=O)NCCCN5CCCCC5)ccc4n3n2)c(F)c1]

Compound 2-(2-fluoro-4-(methylcarbamoyl)phenyl)-N-(3-(piperidin-1-yl)propyl)benzo[4,5]thiazolo[3,2-b][1,2,4]triazole-6-carboxamide formate was prepared from 2-(2-fluoro-4-(methylcarbamoyl)phenyl)benzo[4,5]thiazolo[3,2-b][1,2,4]triazole-6-carboxylic acid (180 mg, 0.486 mmol) and 3-(piperidin-1-yl)propan-1-amine (104 mg, 0.731 mmol) following a similar procedure to that described for the synthesis of 2-bromo-N-(3-(piperidin-1-yl)propyl)benzo[d]imidazo[2,1-b]thiazole-7-carboxamide, and was isolated as a light yellow solid.

Yield 11 mg (4%). ¹H NMR (400 MHz, DMSO) δ 8.74 (t, J=5.6 Hz, 1H), 8.70-8.65 (m, 2H), 8.29-8.24 (m, 1H), 8.23 (s, 1H, formic acid), 8.17-8.09 (m, 2H), 7.89-7.80 (m, 2H), 3.36-3.28 (m, 2H), 2.83 (d, J=4.4 Hz, 3H), 2.47-2.36 (m, 6H), 1.80-1.68 (m, 2H), 1.58-1.48 (m, 4H), 1.44-1.34 (m, 2H). ¹⁹F NMR (376 MHz, DMSO) δ −110.74. m/z: [ESI⁺] 495 (M+H)⁺ (C₂₅H₂₇FN₆02S).

### 2-(2-fluoro-4-(methylcarbamoyl)phenyl)-N-(3-(4-fluoropiperidin-1-yl)propyl)benzo[4,5]thiazolo[3,2-b][1,2,4]triazole-6-carboxamide hemi-formate (Compound 389)

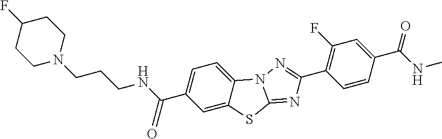 [SMILES: CNC(=O)c1ccc(-c2nc3sc4cc(C(=O)NCCCN5CCC(F)CC5)ccc4n3n2)c(F)c1]

Compound 2-(2-fluoro-4-(methylcarbamoyl)phenyl)-N-(3-(4-fluoropiperidin-1-yl)propyl)benzo[4,5]thiazolo[3,2-b][1,2,4]triazole-6-carboxamide hemi-formate was prepared from 2-(2-fluoro-4-(methylcarbamoyl)phenyl)benzo[4,5]thiazolo[3,2-b][1,2,4]triazole-6-carboxylic acid (200 mg, 0.540 mmol) and 3-(4-fluoropiperidin-1-yl)propan-1-amine di-hydrochloride (151 mg, 0.648 mmol) following a similar procedure to that described for the synthesis of 2-bromo-N-(3-(piperidin-1-yl)propyl)benzo[d]imidazo[2,1-b]thiazole-7-carboxamide, and was isolated as a brown yellow solid.

Yield 14 mg (5%). ¹H NMR (400 MHz, DMSO) δ 8.77-8.64 (m, 3H), 8.32 (s, 1.23H, formic acid), 8.29-8.21 (m, 1H), 8.18-8.08 (m, 2H), 7.89-7.79 (m, 2H), 4.80-4.70 (m, 0.5H), 4.68-4.56 (m, 0.5H), 3.33 (d, J=6.8 Hz, 2H), 2.83 (d, J=4.4 Hz, 3H), 2.61-2.58 (m, 2H), 2.41-2.35 (m, 2H), 2.34-2.25 (m, 2H), 1.93-1.78 (m, 2H), 1.77-1.63 (m, 4H). ¹⁹F NMR (376 MHz, DMSO) δ −110.73. Fluorine signal of 4-fluoropiperidine not observed. m/z: [ESI⁺] 513 (M+H)⁺ (C₂₅H₂₆F₂N₆02S).

### N-(3-(diethylamino)propyl)-2-(4-(methylcarbamoyl)phenyl)benzo[4,5]thiazolo[3,2-b][1,2,4]triazole-6-carboxamide (Compound 328)

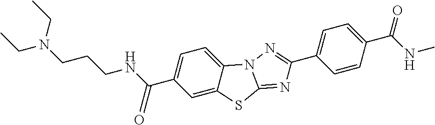 [SMILES: CCN(CC)CCCNC(=O)c1ccc2c(c1)sc1nc(-c3ccc(C(=O)NC)cc3)nn12]

Compound N-(3-(diethylamino)propyl)-2-(4-(methylcarbamoyl)phenyl)benzo[4,5]thiazolo[3,2-b][1,2,4]triazole-6-carboxamide was prepared from 2-(4-(methylcarbamoyl)phenyl)benzo[4,5]thiazolo[3,2-b][1,2,4]triazole-6-carboxylic acid (250 mg, 0.709 mmol) and N′,N¹-diethylpropane-1,3-diamine (120 mg, 0.921 mmol) following a similar procedure to that described for the synthesis of 2-bromo-N-(3-(piperidin-1-yl)propyl)benzo[d]imidazo[2,1-b]thiazole-7-carboxamide, and was isolated as a white solid.

Yield 54 mg (16%). ¹H NMR (400 MHz, DMSO) δ 8.71 (t, J=5.6 Hz, 1H), 8.66 (d, J=1.6 Hz, 1H), 8.58 (q, J=4.4 Hz, 1H), 8.23 (d, J=8.4 Hz, 2H), 8.16-8.07 (m, 2H), 8.00 (d, J=8.4 Hz, 2H), 3.33-3.28 (m, 2H), 2.82 (d, J=4.4 Hz, 3H), 2.48-2.41 (m, 6H), 1.72-1.60 (m, 2H), 0.95 (t, J=7.2 Hz, 6H). m/z: [ESI⁺] 465 (M+H)⁺. (C₂₄H₂₈N₆O₂S).

### 2-(4-(methylcarbamoyl)phenyl)-N-(3-(piperidin-1-yl)propyl)benzo[4,5]thiazolo[3,2-b][1,2,4]triazole-6-carboxamide (Compound 319)

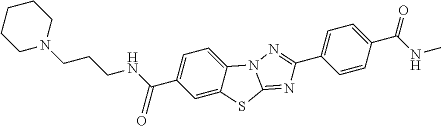 [SMILES: CNC(=O)c1ccc(-c2nc3sc4cc(C(=O)NCCCN5CCCCC5)ccc4n3n2)cc1]

Compound 2-(4-(methylcarbamoyl)phenyl)-N-(3-(piperidin-1-yl)propyl)benzo[4,5]thiazolo[3,2-b][1,2,4]triazole-6-carboxamide was prepared from 2-(4-(methylcarbamoyl)phenyl)benzo[4,5]thiazolo[3,2-b][1,2,4]triazole-6-carboxylic acid (250 mg, 0.709 mmol) and 3-(piperidin-1-yl)propan-1-amine (131 mg, 0.921 mmol) following a similar procedure to that described for the synthesis of 2-bromo-N-(3-(piperidin-1-yl)propyl)benzo[d]imidazo[2,1-b]thiazole-7-carboxamide, and was isolated as an off-white solid.

Yield 48 mg (14%). ¹H NMR (400 MHz, DMSO) δ 8.72 (t, J=5.6 Hz, 1H), 8.66 (d, J=1.6 Hz, 1H), 8.58 (q, J=4.4 Hz, 1H), 8.26-8.20 (m, 2H), 8.16-8.08 (m, 2H), 8.04-7.96 (m, 2H), 3.33-3.29 (m, 2H), 2.82 (d, J=4.4 Hz, 3H), 2.36-2.25 (m, 6H), 1.75-1.65 (m, 2H), 1.53-1.45 (m, 4H), 1.42-1.33 (m, 2H). m/z: [ESI⁺] 477 (M+H)⁺. (C₂₅H₂₈N₆O₂S).

### 2-(4-((2-(3-(but-3-yn-1-yl)-3H-diazirin-3-yl)ethyl)carbamoyl)phenyl)-N-(3-(diethylamino)propyl)benzo[d]imidazo[2,1-b]thiazole-7-carboxamide hemi-formate (Compound 322)

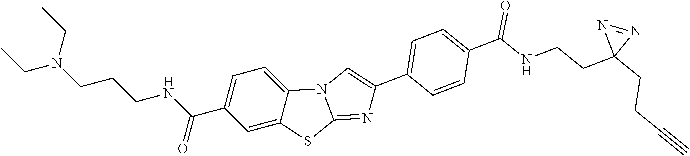 [SMILES: C#CCCC1(CCNC(=O)c2ccc(-c3cn4c(n3)sc3cc(C(=O)NCCCN(CC)CC)ccc34)cc2)N=N1]

Compound 2-(4-((2-(3-(but-3-yn-1-yl)-3H-diazirin-3-yl)ethyl)carbamoyl)phenyl)-N-(3-(diethylamino)propyl)benzo[d]imidazo[2,1-b]thiazole-7-carboxamide hemi-formate was prepared from 4-(7-((3-(diethylamino)propyl)carbamoyl)benzo[d]imidazo[2,1-b]thiazol-2-yl)benzoic acid (200 mg, 0.444 mmol) and 2-(3-(but-3-yn-1-yl)-3H-diazirin-3-yl)ethan-1-amine (61 mg, 0.445 mmol) following a similar procedure to that described for the synthesis of 2-bromo-N-(3-(piperidin-1-yl)propyl)benzo[d]imidazo[2,1-b]thiazole-7-carboxamide, and was isolated as a white solid.

Yield 45 mg (17%). ¹H NMR (300 MHz, DMSO) δ 8.96 (s, 1H), 8.69 (t, J=5.7 Hz, 1H), 8.55-8.47 (m, 2H), 8.26 (s, 0.63H, formic acid), 8.12-8.01 (m, 2H), 7.98 (d, J=8.4 Hz, 2H), 7.93 (d, J=8.4 Hz, 2H), 3.41-3.30 (m, 2H), 3.25-3.16 (m, 2H), 2.87 (t, J=2.7 Hz, 1H), 2.62-2.50 (m, 6H), 2.05 (dt, J=2.7, 7.2 Hz, 2H), 1.81-1.62 (m, 6H), 1.01 (t, J=7.2 Hz, 6H). m/z: [ESI⁺] 570 (M+H)⁺. (C₃₁H₃₅N₇O₂S).

### Example 6

Synthetic details of Additional Compounds of the Invention Synthesis of (S)-2-(2-fluoro-4-(tetrahydrofuran-2-yl)phenyl)-N-(3-(4-fluoropiperidin-1-yl)propyl)benzo[d]imidazo[2,1-b]thiazole-7-carboxamide (Compound 428S)

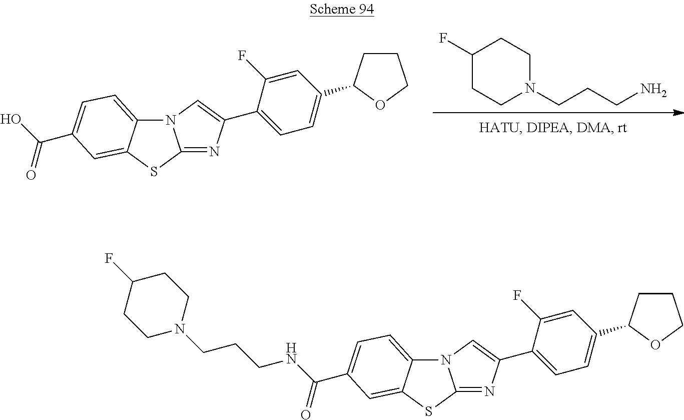 [SMILES: O=C(O)c1ccc2c(c1)sc1nc(-c3ccc([C@@H]4CCCO4)cc3F)cn12 ; NCCCN1CCC(F)CC1 ; O=C(NCCCN1CCC(F)CC1)c1ccc2c(c1)sc1nc(-c3ccc([C@@H]4CCCO4)cc3F)cn12]

To a stirred solution of (S)-2-(2-fluoro-4-(tetrahydrofuran-2-yl)phenyl)benzo[d]imidazo[2,1-b]thiazole-7-carboxylic acid (110 mg, 0.288 mmol) and N,N,N′,N′-tetramethyl-O-(7-azabenzotriazol-1-yl)uronium hexafluorophospate (164 mg, 0.431 mmol) in N,N-dimethylacetamide (5 mL) were added N-ethyl-N-isopropylpropan-2-amine (112 mg, 0.867 mmol) and 3-(4-fluoropiperidin-1-yl)propan-1-amine (92 mg, 0.574 mmol) at room temperature under a nitrogen atmosphere. The reaction solution was stirred for 3 h at room temperature under a nitrogen atmosphere and was purified by reverse phase flash chromatography with the following conditions: Column: Spherical C18, 20-40 um, 120 g; Mobile Phase A: water (10 mM NH₄HCO₃); Mobile Phase B: acetonitrile; Flow rate: 80 mL/min; Gradient: 40% B-60% B in 20 min; Detector: 254 nm. The fractions containing desired product were collected at 50% B and concentrated under reduced pressure to afford (S)-2-(2-fluoro-4-(tetrahydrofuran-2-yl)phenyl)-N-(3-(4-fluoropiperidin-1-yl)propyl)benzo[d]imidazo[2,1-b]thiazole-7-carboxamide as a white solid.

Yield 30 mg (20%). ¹H NMR (400 MHz, DMSO) δ 8.70 (d, J=3.6 Hz, 1H), 8.61 (t, J=5.6 Hz, 1H), 8.49 (d, J=1.6 Hz, 1H), 8.27 (d, J=8.4 Hz, 1H), 8.11 (dd, J=8.0 Hz, 1H), 8.01 (dd, J=1.6, 8.4 Hz, 1H), 7.27 (d, J=2.8 Hz, 1H), 7.25 (s, 1H), 4.91-4.84 (m, 1H), 4.77-4.58 (m, 1H), 4.07-3.97 (m, 1H), 3.89-3.78 (m, 1H), 3.36-3.29 (m, 2H), 2.56-2.48 (m, 2H), 2.40-2.24 (m, 5H), 2.01-1.91 (m, 2H), 1.91-1.77 (m, 2H), 1.76-1.64 (m, 5H). ¹⁹F NMR (376 MHz, CD₃OD) δ −115.13. m/z: [ESI⁺] 525 (M+H)⁺. (C₂₈H₃₀F₂N₄O₂S)

### Synthesis of (R)-2-(2-fluoro-4-(tetrahydrofuran-2-yl)phenyl)-N-(3-(4-fluoropiperidin-1-yl)propyl)benzo[d]imidazo[2,1-b]thiazole-7-carboxamide (Compound 428R)

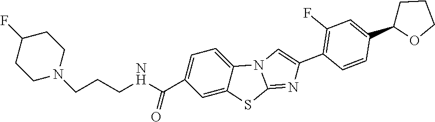 [SMILES: O=C(NCCCN1CCC(F)CC1)c1ccc2c(c1)sc1nc(-c3ccc([C@H]4CCCO4)cc3F)cn12]

Compound (R)-2-(2-fluoro-4-(tetrahydrofuran-2-yl)phenyl)-N-(3-(4-fluoropiperidin-1-yl)propyl)benzo[d]imidazo[2,1-b]thiazole-7-carboxamide was prepared from (R)-2-(2-fluoro-4-(tetrahydrofuran-2-yl)phenyl)benzo[d]imidazo[2,1-b]thiazole-7-carboxylic acid (100 mg, 0.261 mmol) and 3-(4-fluoropiperidin-1-yl)propan-1-amine (84 mg, 0.524 mmol) following a similar procedure to that described for the synthesis of (S)-2-(2-fluoro-4-(tetrahydrofuran-2-yl)phenyl)-N-(3-(4-fluoropiperidin-1-yl)propyl)benzo[d]imidazo[2,1-b]thiazole-7-carboxamide, and was isolated as a white solid.

Yield 30 mg (22%). ¹H NMR (400 MHz, DMSO) δ 8.70 (d, J=3.6 Hz, 1H), 8.61 (t, J=5.6 Hz, 1H), 8.49 (d, J=1.6 Hz, 1H), 8.27 (d, J=8.4 Hz, 1H), 8.11 (dd, J=8.0 Hz, 1H), 8.01 (dd, J=1.6, 8.4 Hz, 1H), 7.27 (d, J=2.8 Hz, 1H), 7.25 (s, 1H), 4.91-4.84 (m, 1H), 4.77-4.58 (m, 1H), 4.07-3.97 (m, 1H), 3.89-3.78 (m, 1H), 3.36-3.29 (m, 2H), 2.56-2.48 (m, 2H), 2.40-2.24 (m, 5H), 2.01-1.91 (m, 2H), 1.91-1.77 (m, 2H), 1.76-1.64 (m, 5H). ¹⁹F NMR (376 MHz, CD₃OD) δ −115.13. m/z: [ESI⁺] 525 (M+H)⁺. (C₂₈H₃₀F₂N₄O₂S)

### Synthesis of (S)-2-(2-fluoro-4-(5-oxopyrrolidin-2-yl)phenyl)-N-(3-(4-fluoropiperidin-1-yl)propyl)benzo[d]imidazo[2,1-b]thiazole-7-carboxamide (Compound 432S)

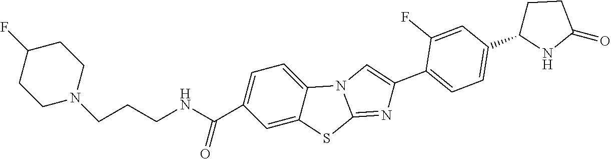 [SMILES: O=C1CC[C@@H](c2ccc(-c3cn4c(n3)sc3cc(C(=O)NCCCN5CCC(F)CC5)ccc34)c(F)c2)N1]

Compound (S)-2-(2-fluoro-4-(5-oxopyrrolidin-2-yl)phenyl)-N-(3-(4-fluoropiperidin-1-yl)propyl)benzo[d]imidazo[2,1-b]thiazole-7-carboxamide was prepared from (S)-2-(2-fluoro-4-(5-oxopyrrolidin-2-yl)phenyl)benzo[d]imidazo[2,1-b]thiazole-7-carboxylic acid (140 mg, 0.354 mmol) and 3-(4-fluoropiperidin-1-yl)propan-1-amine (68 mg, 0.424 mmol) following a similar procedure to that described for the synthesis of (S)-2-(2-fluoro-4-(tetrahydrofuran-2-yl)phenyl)-N-(3-(4-fluoropiperidin-1-yl)propyl)benzo[d]imidazo[2,1-b]thiazole-7-carboxamide, and was isolated as a white solid.

Yield 14 mg (7%). ¹H NMR (400 MHz, DMSO) δ 8.72 (d, J=3.6 Hz, 1H), 8.61 (t, J=5.6 Hz, 1H), 8.49 (d, J=1.6 Hz, 1H), 8.27 (d, J=8.4 Hz, 1H), 8.20-8.10 (m, 2H), 8.07-7.98 (m, 1H), 7.32-7.21 (m, 2H), 4.79-4.55 (m, 2H), 3.39-3.32 (m, 2H), 2.59-2.52 (m, 3H), 2.41-2.33 (m, 2H), 2.33-2.22 (m, 4H), 1.93-1.77 (m, 3H), 1.77-1.62 (m, 4H). ¹⁹F NMR (376 MHz, DMSO) δ −113.26. m/z: [ESI⁺] 538 (M+H)⁺. (C₂₈H₂₉F₂N₅O₂S)

### Synthesis of (R)-2-(2-fluoro-4-(5-oxopyrrolidin-2-yl)phenyl)-N-(3-(4-fluoropiperidin-1-yl)propyl)benzo[d]imidazo[2,1-b]thiazole-7-carboxamide (Compound 432R)

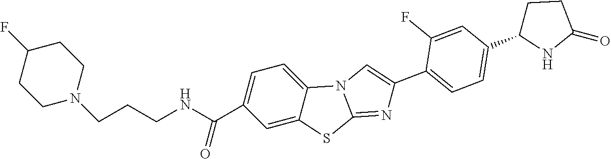 [SMILES: O=C1CC[C@@H](c2ccc(-c3cn4c(n3)sc3cc(C(=O)NCCCN5CCC(F)CC5)ccc34)c(F)c2)N1]

Compound (R)-2-(2-fluoro-4-(5-oxopyrrolidin-2-yl)phenyl)-N-(3-(4-fluoropiperidin-1-yl)propyl)benzo[d]imidazo[2,1-b]thiazole-7-carboxamide was prepared from (R)-2-(2-fluoro-4-(5-oxopyrrolidin-2-yl)phenyl)benzo[d]imidazo[2,1-b]thiazole-7-carboxylic acid (100 mg, 0.253 mmol) and 3-(4-fluoropiperidin-1-yl)propan-1-amine (61 mg, 0.381 mmol) following a similar procedure to that described for the synthesis of (S)-2-(2-fluoro-4-(tetrahydrofuran-2-yl)phenyl)-N-(3-(4-fluoropiperidin-1-yl)propyl)benzo[d]imidazo[2,1-b]thiazole-7-carboxamide, and was isolated as a white solid.

Yield 12 mg (9%). ¹H NMR (400 MHz, DMSO) δ 8.72 (d, J=3.6 Hz, 1H), 8.61 (t, J=5.6 Hz, 1H), 8.49 (d, J=1.6 Hz, 1H), 8.27 (d, J=8.4 Hz, 1H), 8.20-8.10 (m, 2H), 8.07-7.98 (m, 1H), 7.32-7.21 (m, 2H), 4.79-4.55 (m, 2H), 3.39-3.32 (m, 2H), 2.59-2.52 (m, 3H), 2.41-2.33 (m, 2H), 2.33-2.22 (m, 4H), 1.93-1.77 (m, 3H), 1.77-1.62 (m, 4H). ¹⁹F NMR (376 MHz, DMSO) δ −113.26. m/z: [ESI⁺] 538 (M+H)⁺. (C₂₈H₂₉F₂N₅O₂S)

### Example 7

### Biological Activity of Compounds of the Invention

The biological activity results of compounds of the invention are summarized in Table 2.

TABLE 2
Cellular −LogEC₅₀ values of compounds of
the invention in the immunofluorescence assay.

| Compound | Myc Efficacy |
|---|---|
| No. | (−LogEC₅₀) |
| 100 | ++ |
| 101 | +++ |
| 102 | + |
| 103 | +++ |
| 104 | + |
| 105 | + |
| 106 | + |
| 107 | ++ |
| 108 | ++ |
| 109 | ++ |
| 110 | − |
| 111 | ++ |
| 112 | + |
| 114 | ++ |
| 115 | + |
| 116 | ++ |
| 117 | + |
| 118 | +++ |
| 119 | ++ |
| 122 | + |
| 123 | ++ |
| 124 | ++ |
| 125 | ++ |
| 126 | ++ |
| 127 | + |
| 129 | ++ |
| 130 | ++ |
| 131 | ++ |
| 132 | ++ |
| 133 | ++ |
| 134 | + |
| 135 | + |
| 136 | ++ |
| 137 | ++ |
| 138 | ++ |
| 139 | + |
| 140 | ++ |
| 141 | + |
| 142 | ++ |
| 143 | ++ |
| 144 | + |
| 145 | ++ |
| 149 | ++ |
| 150 | + |
| 151 | + |
| 152 | ++ |
| 153 | + |
| 154 | + |
| 155 | ++ |
| 156 | ++ |
| 157 | ++ |
| 158 | ++ |
| 159 | + |
| 160 | + |
| 161 | +++ |
| 162 | − |
| 163 | +++ |
| 164 | +++ |
| 165 | + |
| 166 | +++ |
| 167 | + |
| 168 | ++ |
| 169 | ++ |
| 170 | ++ |
| 171 | ++ |
| 172 | − |
| 173 | ++ |
| 174 | +++ |
| 175 | − |
| 176 | − |
| 177 | + |
| 178 | + |
| 179 | + |
| 180 | + |
| 181 | ++ |
| 182 | − |
| 183 | + |
| 184 | + |
| 185 | ++ |
| 186 | + |
| 187 | − |
| 188 | + |
| 189 | − |
| 190 | + |
| 191 | + |
| 192 | − |
| 193 | + |
| 194 | + |
| 195 | − |
| 196 | + |
| 197 | + |
| 198 | + |
| 199 | + |
| 200 | + |
| 201 | + |
| 202 | − |
| 203 | + |
| 204 | + |
| 205 | + |
| 206 | − |
| 207 | ++ |
| 208 | + |
| 209 | + |
| 210 | + |
| 211 | − |
| 212 | + |
| 213 | + |
| 214 | + |
| 215 | − |
| 216 | + |
| 217 | + |
| 218 | ++ |
| 219 | + |
| 220 | + |
| 221 | + |
| 222 | + |
| 223 | − |
| 224 | − |
| 225 | + |
| 226 | − |
| 227 | ++ |
| 228 | + |
| 229 | + |
| 230 | + |
| 231 | − |
| 232 | − |
| 233 | − |
| 234 | + |
| 235 | + |
| 236 | − |
| 237 | − |
| 238 | − |
| 239 | + |
| 240 | + |
| 241 | + |
| 242 | + |
| 243 | + |
| 244 | − |
| 245 | − |
| 246 | + |
| 247 | − |
| 248 | + |
| 249 | − |
| 250 | + |
| 251 | − |
| 252 | + |
| 253 | + |
| 254 | + |
| 255 | − |
| 256 | − |
| 257 | ++ |
| 258 | + |
| 259 | + |
| 260 | − |
| 261 | − |
| 262 | + |
| 263 | − |
| 264 | +++ |
| 265 | + |
| 266 | + |
| 267 | + |
| 268 | + |
| 269 | + |
| 270 | + |
| 271 | + |
| 272 | − |
| 273 | − |
| 274 | + |
| 275 | − |
| 276 | +++ |
| 277 | ++ |
| 278 | − |
| 279 | − |
| 280 | ++ |
| 281 | ++ |
| 282 | + |
| 283 | + |
| 284 | + |
| 285 | + |
| 286 | ++ |
| 287 | + |
| 288 | +++ |
| 289 | +++ |
| 290 | +++ |
| 291 | + |
| 292 | +++ |
| 293 | ++ |
| 294 | ++ |
| 295 | ++ |
| 296 | + |
| 297 | + |
| 298 | +++ |
| 299 | ++ |
| 300 | +++ |
| 301 | + |
| 303 | + |
| 306 | ++ |
| 307 | ++ |
| 308 | + |
| 309 | ++ |
| 310 | + |
| 312 | ++ |
| 314 | + |
| 315 | ++ |
| 317 | + |
| 318 | ++ |
| 319 | ++ |
| 320 | +++ |
| 321 | ++ |
| 322 | + |
| 323 | +++ |
| 324 | ++ |
| 325 | ++ |
| 326 | ++ |
| 327 | + |
| 328 | ++ |
| 329 | + |
| 330 | + |
| 331 | +++ |
| 332 | +++ |
| 333 | + |
| 334 | ++ |
| 348 | ++ |
| 362 | ++ |
| 363 | ++ |
| 364 | ++ |
| 365 | ++ |
| 368 | +++ |
| 369 | +++ |
| 370 | +++ |
| 371 | ++ |
| 372 | ++ |
| 373 | +++ |
| 374 | +++ |
| 375 | +++ |
| 376 | +++ |
| 378 | +++ |
| 379 | +++ |
| 381 | +++ |
| 383 | ++ |
| 384 | ++ |
| 386 | +++ |
| 387 | +++ |
| 388 | ++ |
| 389 | ++ |
| 390 | ++ |
| 392 | +++ |
| 393 | +++ |
| 394 | ++ |
| 395 | ++ |
| 396 | ++ |
| 397 | ++ |
| 398 | ++ |
| 406 | ++ |
| 412 | +++ |
| 413 | +++ |
| 414 | +++ |
| 415 | +++ |
| 416 | +++ |
| 417 | +++ |
| 418 | +++ |
| 419 | +++ |
| 420 | +++ |
| 421 | ++ |
| 422 | +++ |

Activity (−LogEC50):
− ≤3
+ >3 and <5
++ ≥5 and <6
+++ ≥6

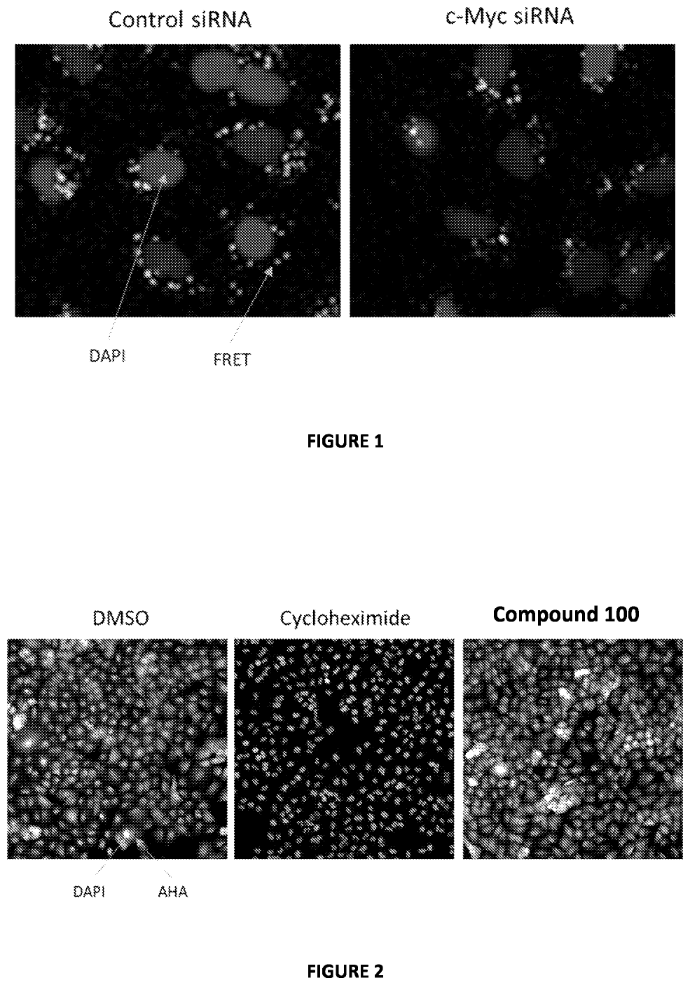

Compounds activity was tested in tumor cell lines expressing c-Myc by using high content image analysis. c-Myc mRNA rate of translation was assays using PSM assay, c-Myc protein levels and intracellular localization were assayed by immunofluorescence using a c-Myc specific antibody and c-Myc mRNA levels and intracellular localization was tested using specific fluorescent probes, as detailed in the Experimental Methods below (Example 9). The di-tRNA translation rate measurement specificity to c-Myc was shown by co-transfecting c-Myc specific siRNA. Transfection of labelled di-tRNA with c-Myc specific siRNA reduced the FRET signal originating from ribosome translating c-Myc, relative to cells transfected with nonrelevant siRNA (FIG. 1).

Compounds did not affect global translation. A549 cells were incubated with active compounds and metabolically labelled with fluorescent methionine for a 4 hour pulse (click-chemistry modified methionine). Cells were fixed and newly synthesized proteins detected by using click-chemistry with a fluorescent detector (FIG. 2). Global ribosome inhibitor, cycloheximide (CHX) completely reduced incorporation of modified methionine (FIG. 2, compare middle and left panels). However, a reperentative compound did not inhibit incorporation of modified methionine, indicating that global translation is not affected by the compounds (FIG. 2, compare right and left panels, respectively).

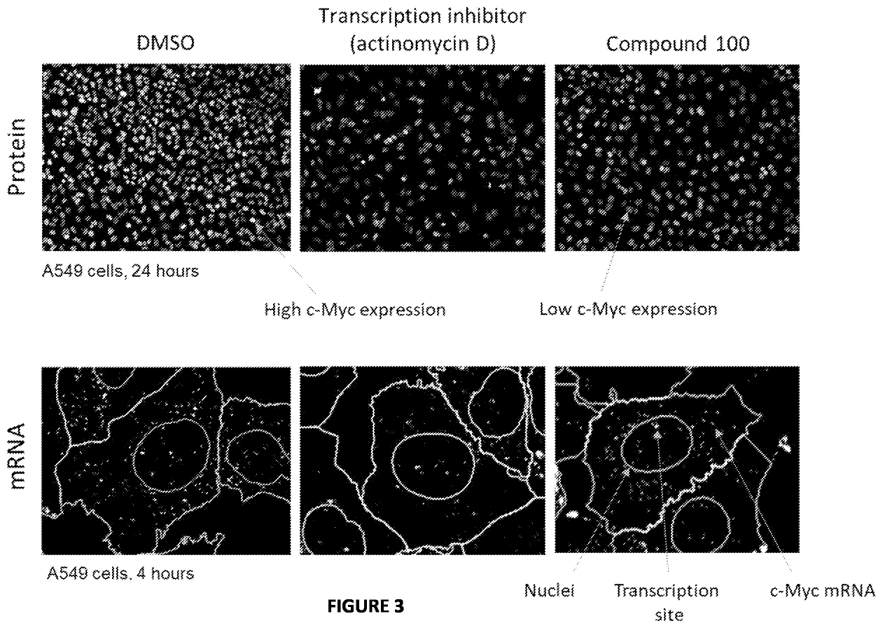

Compounds reduced c-Myc protein accumulation in A549 cells without affecting c-Myc transcription. A549 cells were incubated with compounds for 24 hours (FIG. 3, upper panel) and c-Myc protein detected by immunofluorescence. In parallel, A549 cells were incubated with compounds for 4 hours and c-Myc mRNA was visualized by micrscopy using c-Myc mRNA specific fluorescent-tagged probes (FIG. 3, lower panel). Both a general transcription inhibitor, Actinomycin D, and compounds of the invention, reduced c-Myc protein (FIG. 3, upper panel). Actinomycin D inhibited transcription site (FIG. 3, middle lower panel, spots inside the nucleus) and mRNA accumulation in the cytoplasm (FIG. 3, middle lower panel, spots in the cytoplasm). However, compound treated cells did not affect transcription site intensity or number (FIG. 3, right lower panel, spots inside the nucleus are evident), but did affect steady state levels of mRNA in the cytoplasm (FIG. 3, right lower panel, reduction of spots in the cytoplasm relative to DMSO control). This indicates that compounds of the invention affect c-Myc steady state mRNA levels, either by affecting turn over rate of c-Myc mRNA, or by inhibiting its recruitment by ribosomes.

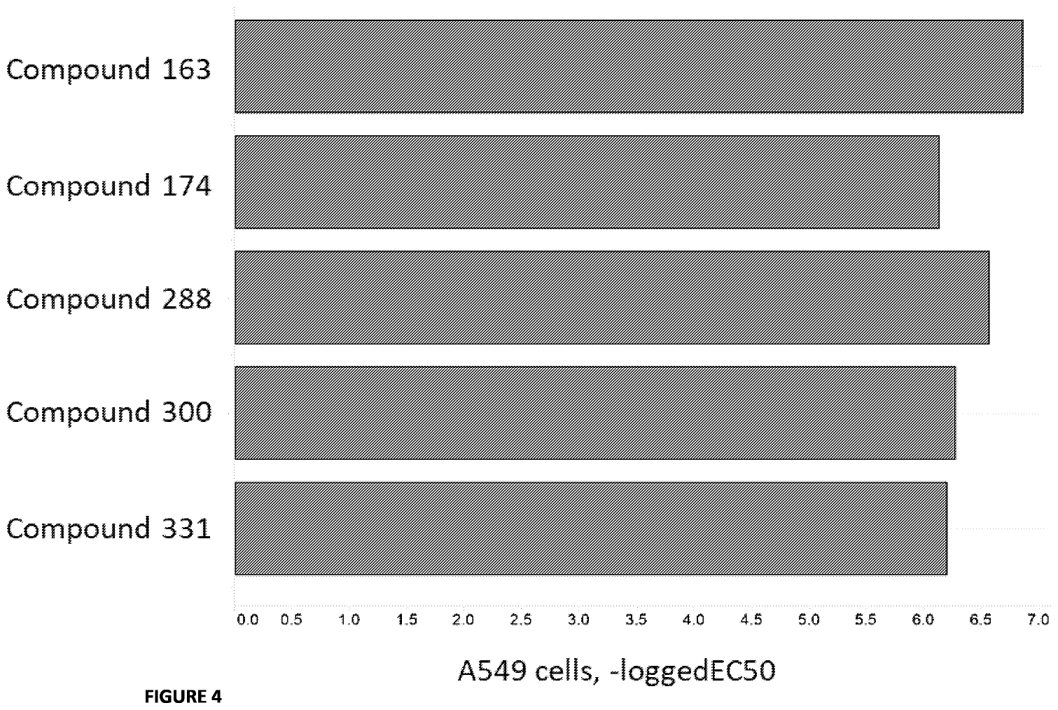

A549 human non-small cell lung carcinoma cells were treated for 24 hours with increasing compound concentration, cells were fixed and stained with a nuclei stain (DAPI) and anti-c-Myc fluorescent antibody. The c-Myc signal was quantified by image analysis, and data was exported and analyzed using TIBCO Spotfire® (TIBCO Corporation). Dose response curves were generated and fitted with logaristic regression to calculate potency (EC₅₀ values). Potency values are presented in Table 2 for all compounds and are shown for selected compounds (FIG. 4).

### Example 8

### In Vivo Activity of Compounds of the Invention

### A549 Xenograft Model in Nude Mice.

NMRI nude female mice of 6-8 weeks of age were acclimated after shipping for >4 days. A549 cells, 5×10⁶ in 100 ul Matrigel:PBS (50:50), were subcutaneously injected into flanks of mice. When the tumor size reached 80 to 200 mm³, mice were grouped with similar average tumor size in each group, 10 animals per group. Compounds were dissolved in 10% DMSO, 10% Solutol, 80% water. Compounds were given i.p. for 49 days at 3 mg/kg twice a week. Caliper measurement of tumor size was done twice a week.

Compound 332 inhibited c-Myc-dependent tumor growth in-vivo. FIG. 5 shows the relative tumor volumes of A549 xenografts in NMRI female nude mice after they were treated with compound 332, 3 mg/kg, twice a week, for 49 days. Error bars represent median±SEM, n=10 mice at each time point and analyzed by one-tailed T-TEST in Prism for *p<0.05.

### Example 9

### Experimental Methods

High Content Screen for the Identification of c-Myc Modulators

Compound effect on translation of c-Myc in A549, human non-small cell lung carcinoma cell line, was conducted using specific PSM assay using tRNAgln and tRNAser isoacceptors, as described below. A library of diverse small molecules, 90,000 compounds, was used at a final concentration of 30 μM. Image and data analyses were conducted using Anima's proprietary algorithms. False positive and toxic compounds were eliminated. A total of 3,307 compounds were identified as hits, compounds which increased or decreased the FRET signal generated by ribosomes during c-Myc translation.

Positive hits were re-screened in the specific PSM assay, using tRNAgln and tRNAser. Hits were scored using Anima's proprietary algorithms, and 348 compounds, which selectively inhibited c-Myc synthesis in specific PSM assay, were selected as confirmed hits. These compounds were purchased as powder to confirm activity. Re-purchased hits were tested in the specific PSM assay (tRNAgln-tRNAser) and anti-c-Myc immunofluorescence, and in counter assays to eliminate global translation modulators: (1) bulk tRNA and (2) metabolic labeling using Click-IT™ AHA (L-Azidohomoalanine).

### Cell Culture

A549 cells (ATCC® CCL-185™) were maintained in DMEM low glucose medium (Biological Industries, Cat. 01-050-1A), containing 10% fetal bovine serum, 1% L-glutamine and 1% penicillin-streptomycin solution.

SK-N-F1 cells (ATCC® CRL-2142™) were maintained in DMEM high glucose medium (Biological Industries, Cat. 01-055-1A), containing 10% fetal bovine serum, 2% L-glutamine, 1% penicillin-streptomycin solution, 1% sodium pyruvate and 1% non-essential amino acids.

Specific tRNA (tRNA Isoacceptor) Isolation and Labeling

The specific tRNAgln (TTG) and tRNAser (CGA) were isolated for from baker's yeast (Roche) using biotinylated oligos complimentary to sequences encompassing the D-loop and anti-codon. The biotinylated oligos were mixed with total yeast tRNA and heated up to 82° C. for 10 min, followed by addition of TMA buffer (20 mM Tris, pH 7.6, 1.8M tetramethylammonium chloride, 0.2 mM EDTA). The mixture was incubated at 68° C. for 10 min, and annealed by slow cooling to 37° C. tRNA:DNA oligo mixture then was incubated with streptavidin linked agarose beads at room temperature for 30 min while shaking. Unbound tRNA and tRNA:DNA complexes were removed by centrifugation and beads washed with 10 mM Tris-HCl (pH 7.6). The target tRNA was eluted from the resin by incubation at 45° C. or 55° C. for 7 min followed by centrifugation and collection of the supernatant to clean tubes.

The purity of the isolated tRNA isoacceptors was confirmed using fluorescent polarization assay. Purified tRNA was annealed to a complementary oligo tagged at the 3′-end with Cy3. The annealed purified tRNA isoacceptor FP signal was compared to the signal derived from annealing of a tRNA isoacceptor oligo annealed to the same Cy3-oligo. Samples with more than 80% purity were selected for labeling.

The dihydrouridines of the target tRNAs or total yeast tRNA were labeled as described in U.S. Pat. No. 8,785,119. Labeled tRNAs were purified by reverse phase HPLC and eluted with an ethanol gradient.

### Protein Synthesis Monitoring (PSM) Assays

Cy3 and Cy5 Labeled tRNA, bulk or specific, were transfected with 0.4 μl HiPerFect (Qiagen) per 384 well. First, HiPerFect was mixed with DMEM and incubated for 5 min; next, 6 nanograms Cy3-labeled tRNAgln and 6 ng Cy5-labeled tRNAser (or 9 ng each Cy3 and Cy5-labelled bulk tRNA) were diluted in 1×PBS and then added to the HiPerFect:DMEM cocktail and incubated at room temperature for 10 min. The transfection mixture was dispersed automatically into 384-well black plates. Cells were then seeded at 3,500 cells per well in complete culture medium and incubated at 37° C., 5% CO₂. Forty-eight hours after transfection compounds were added at a final concentration of 30 μM. Four hours post-treatment, cells were fixed with 4% paraformaldehyde and images were captured with Operetta microscope (Perkin Elmer) using ×20 high NA objective lens.

### Metabolic Labeling Assay

A549 cells were seeded at 3,200 cells per well in complete culture medium. Plates were incubated at 37° C., 5% CO₂ overnight. After 48 hours of incubation, the growth medium was aspirated, and cells were washed three times with HBSS. Metabolic labeling medium DMEM (-Cys -Met), containing 10% dialyzed FBS, 1% pencillin-streptomycin and 1% L-glutamine was added to the cells for 30 min. Then medium was replaced by metabolic labeling medium containing 25 μM L-Azidohomoalanine (AHA, ThermoFisher) and tested compounds at a final concentration of 30 μM, and cells were incubated for 4 hours at 37° C., 5% CO₂. Cells were washed by HBSS at 37° C. for 15 min before fixing with 4% paraformaldehyde. Cells were washed twice with 3% BSA in PBS before permeabilization with 0.5% Triton X-100 in PBS for 20 min. The AHA staining with Alexa Fluor™ 555 alkyne was performed according to the manufacturer protocol. Images were captured with Operetta microscope (Perkin Elmer) using ×20 high NA objective lens.

c-Myc Immunofluorescence Assay

A549 cells were grown in 384-wells plates (Perkin Elmer, Cat. 6057300) for 48 hours, treated with compounds and then fixed for 20 min in 4% paraformaldehyde. After that permeabilization was done using 0.1% Triton X-100 in PBS for 20 min. Primary anti-c-Myc antibody (Abcam, ab32072) staining was performed for 90 min at room temperature. Cells then were washed twice with PBS and incubated with secondary antibody (Abcam, ab150075) for 90 min at room temperature. Nuclei were stained with DAPI for 10 min and washed twice with PBS.

Cell images were taken with Operetta (Perkin Elmer, USA), a wide-field fluorescence microscope at 20× magnification. After acquisition, the images were transferred to Columbus software (Perkin-Elmer) for image analysis. In Columbus, cells were identified by their nucleus, using the “Find Nuceli” module and cytoplasm was detected based on the secondary antibody channel. Subsequently, the fluorescent signal was enumerated in the identified cell region. Then data was exported to a data analysis and visualization software, Tibco Spotfire, USA.

### Fluorescent In Situ Hybridization (FISH) Assay

A549 cells were grown in 384-wells plates (Perkin Elmer, Cat. 6057300) for 48 hours, treated with compounds for 4 hours and then fixed for 20 min in 4% paraformaldehyde. Next day, permeabilization was done for 90 min at 4° C., using 70% ethanol. Then, the cells were incubated for 10 min with 10% formamide in 10% saline-sodium citrate. Fluorescently labeled custom DNA probes that target c-Myc (Cy3, BioSearch Technologies, Cat. SMF-1063-5) and GAPDH (Cy5, BioSearch Technologies, Cat. SMF-2019-1) mRNAs were hybridized overnight at 37° C. in a dark chamber in 10% formamide. The next day, cells were washed twice with 10% formamide for 30 min. Next, nuclei were counterstained with DAPI (SIGMA, Cat. 5MG-D9542) and then cells were washed twice with PBS. FISH experiments were performed according to the probes manufacturer's protocol for adherent cells.

Following RNA FISH experiments, images of cells were taken with Operetta (Perkin Elmer, USA), a wide-field fluorescence microscope at ×63 magnification. After acquisition, the images were transferred to Columbus software for image analysis. In Columbus, cells were identified by their nucleus, using the “Find Nuceli” module, cytoplasm was detected based on the FISH-channel, and single mRNAs in the cytoplasm and transcription sites in the nucleus were detected using “Find Spots” module. Subsequently, fluorescent signals were collected for each channel in the identified regions: nucleus, cytoplasm and spots. Data was exported to a data analysis and visualization software, Tibco Spotfire, USA.

### A549 Xenograft Model in Nude Mice.

NMRI nude female mice of 6-8 weeks of age were acclimated after shipping for >4 days. A549 cells, 5×10⁶ in 100 ul Matrigel:PBS (50:50), were subcutaneously injected into flanks of mice. When the tumor size reached 80 to 200 mm³, mice were grouped with similar average tumor size in each group, 10 animals per group. Compounds were dissolved in 10% DMSO, 10% Solutol, 80% water. Compounds were given i.p. for 49 days at 3 mg/kg twice a week. Caliper measurement of tumor size was done twice a week.

What is claimed:

1. A compound represented by the structure of formula I(h): 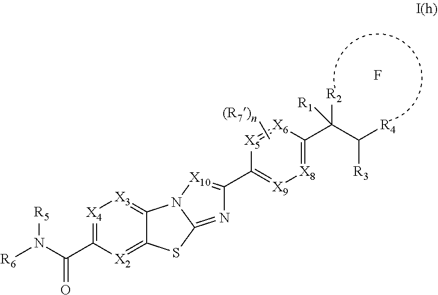
wherein
Ring F is absent or is a substituted or unsubstituted, saturated or unsaturated, 4-8 membered heterocyclic ring, (e.g., pyrrolidine, pyrrolidin-2-one, pyridine, piperidine, imidazole, pyrimidine, triazole, oxadiazole, pyrazole);
R₁ and R₂ are each independently H, F, Cl, Br, I, OH, SH, or CF₃, substituted or unsubstituted C₁-C₅ alkyl, C₁-C₅ linear or branched, or C₃-C₈ cyclic haloalkyl, substituted or unsubstituted C₁-C₅ linear or branched, or C₃-C₈ cyclic alkoxy;
or R₁ and R₂ are joined to form a C₃-C₈ carbocyclic or heterocyclic ring (e.g., cyclopropyl);
or R₂ and R₄ are joined to form Ring F as defined above (e.g., pyrrolidine, pyridine, pyrimidine, triazole, oxadiazole, pyrazole),
wherein if Ring F is aromatic, then R₁ and/or R₃ are absent;
R₃ and R₄ are each independently H, Me, substituted or unsubstituted C₁-C₅ alkyl (e.g., methoxyethylene, methylaminoethyl, aminoethyl), —R₈—O—R₁₀ (e.g., (CH₂)₂—O—CH₃), R₈—N(R₁₀)(R₁₁) (e.g., (CH₂)₂—NH(CH₃)), substituted or unsubstituted C₃-C₈ cycloalkyl (e.g., cyclopropyl), substituted or unsubstituted 5-7 membered heterocyclic ring (e.g., pyrrolidine, methylpyrrolidine, piperidine), or R₂₀; or
R₃ and R₄ are joined to form a 3-8 membered heterocyclic ring (e.g., pyrrolidine, pyrrolidone, 2-oxopyrrolidine, piperidine, morpholine, piperazine, imidazole);
X₂, X₃, and X₄, are each independently nitrogen or CH;
X₅, X₆, X₇, X₈ and X₉ are each independently nitrogen or carbon atoms;
X₁₀ is N, CH, or C(R);
R₅ is H or C₁-C₅ linear or branched alkyl (e.g. methyl);
R₆ is R₈-(substituted or unsubstituted, saturated, unsaturated or aromatic, single, fused or spiro 3-10 membered heterocyclic ring) (e.g., (CH₂)₃-piperidine, (CH₂)₃-4-fluoro-piperidine, (CH₂)₃-piperidine-2-one, (CH₂)₃-4-cyano-piperidine, (CH₂)₃-4-trifluoromethyl-piperidine), R₈—N(R₁₀)(R₁₁) (e.g., (CH₂)₃—N(CH₂CH₃)₂);
R₇′ is each independently H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O-R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl (e.g., isopropyl, methyl, ethyl), C₁-C₅ linear or branched, substituted or unsubstituted alkenyl, C₁-C₅ linear or branched, or C₃-C₈ cyclic haloalkyl (e.g., CHF₂), C₁-C₅ linear or branched, or C₃-C₈ cyclic alkoxy (e.g. methoxy), optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl (e.g., cyclopropyl), substituted or unsubstituted 3-8 membered heterocyclic ring (e.g., morpholine, pyran, oxetane, pyrrolidine, imidazole, piperazine, piperidine, diaoxazole, 2-oxopyrrolidine), substituted or unsubstituted aryl, substituted or unsubstituted benzyl;
R₂₀ is represented by the following structure: 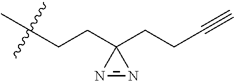 [SMILES: *CCC1(CCC#C)N=N1]
R is H, F, Cl, Br, I, OH, SH, OH, alkoxy, NH(R₁₀), NH—CH₂-cyclopropyl, N(R₁₀)(R₁₁), CF₃, CN, NO₂, COOH, C₁-C₅ linear or branched, substituted or unsubstituted alkyl, methyl, ethyl, CH₂—OH, CH₂—CH₂—OH, C₃-C₈ substituted or unsubstituted cycloalkyl, cyclopropyl, C₁-C₅ linear or branched alkoxy, isopropoxy, C₁-C₅ linear or branched haloalkyl, R₈-aryl, —R₈—O—R₈—O—R₁₀, —R₈—O—R₁₀, —R₈-R₁₀, substituted or unsubstituted aryl, substituted or unsubstituted heteroaryl;
R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl (e.g., methyl, ethyl, CH₂—CH₂—O—CH₂—CH₂—O—CH₃, CH₂—O—CH₂—CH₂—O—CH₃), C₁-C₅ linear or branched alkoxy, C₁-C₅ linear or branched haloalkyl (e.g., CHF₂, CF₃, CF₂CH₃, CH₂CF₃, CF₂CH₂CH₃, CH₂CH₂CF₃, CF₂CH(CH₃)₂, CF(CH₃)—CH(CH₃)₂), R₈-aryl (e.g., CH₂-Ph), —R₈—O—R₈—O—R₁₀ (e.g. (CH₂)₂—O—(CH₂)₂—O—CH₃), —R₈—O—R₁₀, —R₈-R₁₀ (e.g., (CH₂)₂—O—CH₃), substituted or unsubstituted aryl (e.g., phenyl), substituted or unsubstituted heteroaryl (e.g., pyridine (2, 3, and 4-pyridine);
each R₈ is independently [CH₂]ₚ
wherein p is between 1 and 10;
R₉ is [CH]_{q}, [C]_{q}
wherein q is between 2 and 10;
R₁₀ and R₁₁ are each independently H, C₁-C₅ substituted or unsubstituted linear or branched alkyl (e.g., methyl, ethyl, CH₂-cyclopropyl, CH₂—CH₂—O—CH₃, CH₂CF₃, C—C substituted or unsubstituted linear or branched haloalky, CH₂CF₃, C₁-C₅ linear or branched alkoxy (e.g., O—CH₃), R₂₀, C(O)R, or S(O)₂R;
or R₁₀ and R₁₁ are joined to form a substituted or unsubstituted 3-8 membered heterocyclic ring (e.g., piperazine, piperidine),
n is an integer between 0 and 4 (e.g., 1, 2);
or its pharmaceutically acceptable salt, stereoisomer, tautomer, hydrate, N-oxide, reverse amide analog, isotopic variant (e.g., deuterated analog), pharmaceutical product or any combination thereof.

2. The compound of claim 1, wherein R₁ is H; R₃ is H or CH₃; R₂ is H, R₄ is H or cyclopropyl, or R₂ and R₄ are joined to form a pyrrolidine, pyrrolidin-2-one, pyridine, or piperidine; R₇′ is H, CH₃, cyclopropyl, F, Cl, CF₃, or CHF₂; Rn is (CH₂)₃-piperidine, (CH₂)₃-4-fluoro-piperidine, piperidine-2-one, (CH₂)₃-4-cyano-piperidine, (CH₂)₃-4-trifluoromethyl-piperidine, or (CH₂)₃—N(CH₂CH₃)₂; wherein the compound is a substantially pure single stereoisomer; or any combination thereof.

3. The compound of claim 1, selected from the following:

| Compound | |
|---|---|
| No. | Compound Name |
| 163 | 2-(4-(aminomethyl)phenyl)-N-(3-(diethylamino)propyl)benzo[d]imidazo[2,1- |
| | b]thiazole-7-carboxamide |
| 194 | N-(3-(diethylamino)propyl)-2-(4-methylpyridin-2-yl)benzo[d]imidazo[2,1- |
| | b]thiazole-7-carboxamide |
| 218 | N-(3-(diethylamino)propyl)-2-(4-(2,2,2-trifluoro-1- |
| | (methylamino)ethyl)phenyl)benzo[d]imidazo[2,1-b]thiazole-7-carboxamide |
| 268 | 2-(4-(1H-imidazol-2-yl)phenyl)-N-(3-(diethylamino)propyl)benzo[d]imidazo[2,1- |
| | b]thiazole-7-carboxamide |
| 276 | N-(3-(diethylamino)propyl)-2-(4- |
| | ((dimethylamino)methyl)phenyl)benzo[d]imidazo[2,1-b]thiazole-7-carboxamide |
| 288 | N-(3-(diethylamino)propyl)-2-(4- |
| | ((methylamino)methyl)phenyl)benzo[d]imidazo[2,1-b]thiazole-7-carboxamide |
| 289 | N-(3-(diethylamino)propyl)-2-(4-(pyrrolidin-1- |
| | ylmethyl)phenyl)benzo[d]imidazo[2,1-b]thiazole-7-carboxamide |
| 290 | 2-(4-(aminomethyl)phenyl)-N-(3-(piperidin-1-yl)propyl)benzo[d]imidazo[2,1- |
| | b]thiazole-7-carboxamide |
| 292 | N-(3-(diethylamino)propyl)-2-(4-(((2- |
| | methoxyethyl)amino)methyl)phenyl)benzo[d]imidazo[2,1-b]thiazole-7-carboxamide |
| 293 | (R)-N-(3-(diethylamino)propyl)-2-(4-(2,2,2-trifluoro-1- |
| | (methylamino)ethyl)phenyl)benzo[d]imidazo[2,1-b]thiazole-7-carboxamide |
| 294 | (S)-N-(3-(diethylamino)propyl)-2-(4-(2,2,2-trifluoro-1- |
| | (methylamino)ethyl)phenyl)benzo[d]imidazo[2,1-b]thiazole-7-carboxamide |
| 298 | 2-(4-(aminomethyl)-2-fluorophenyl)-N-(3- |
| | (diethylamino)propyl)benzo[d]imidazo[2,1-b]thiazole-7-carboxamide |
| 300 | 2-(4-((cyclopropylamino)methyl)phenyl)-N-(3- |
| | (diethylamino)propyl)benzo[d]imidazo[2,1-b]thiazole-7-carboxamide |
| 318 | 2-(4-(1-aminocyclopropyl)phenyl)-N-(3-(diethylamino)propyl)benzo[d]imidazo[2,1- |
| | b]thiazole-7-carboxamide |
| 330 | N-(3-(diethylamino)propyl)-2-(pyrimidin-4-yl)benzo[d]imidazo[2,1-b]thiazole-7- |
| | carboxamide |
| 331 | 2-(4-((cyclopropylamino)methyl)-2-fluorophenyl)-N-(3- |
| | (diethylamino)propyl)benzo[d]imidazo[2,1-b]thiazole-7-carboxamide |
| 332 | 2-(4-(1-aminocyclopropyl)phenyl)-N-(3-(piperidin-1-yl)propyl)benzo[d]imidazo[2,1- |
| | b]thiazole-7-carboxamide |
| 334 | 2-(4-((cyclopropylamino)methyl)-2,5-difluorophenyl)-N-(3- |
| | (diethylamino)propyl)benzo[d]imidazo[2,1-b]thiazole-7-carboxamide |
| 338 | 2-(4-(aminofluoromethyl)phenyl)-N-(3-(diethylamino)propyl)benzo[d]imidazo[2,1- |
| | b]thiazole-7-carboxamide |
| 339 | N-(3-(diethylamino)propyl)-2-(4-(5-methyl-4H-1,2,4-triazol-3- |
| | yl)phenyl)benzo[d]imidazo[2,1-b]thiazole-7-carboxamide |
| 340 | N-(3-(diethylamino)propyl)-2-(4-(3-methyl-1,2,4-oxadiazol-5- |
| | yl)phenyl)benzo[d]imidazo[2,1-b]thiazole-7-carboxamide |
| 341 | 2-(4-(1H-pyrazol-5-yl)phenyl)-N-(3-(diethylamino)propyl)benzo[d]imidazo[2,1- |
| | b]thiazole-7-carboxamide |
| 342 | N-(3-(diethylamino)propyl)-2-(4-((2-oxopyrrolidin-1- |
| | yl)methyl)phenyl)benzo[d]imidazo[2,1-b]thiazole-7-carboxamide |
| 343 | N-(3-(diethylamino)propyl)-2-(4-(((2- |
| | (methylamino)ethyl)amino)methyl)phenyl)benzo[d]imidazo[2,1-b]thiazole-7- |
| | carboxamide |
| 344 | N-(3-(diethylamino)propyl)-2-(4-(piperidin-1-ylmethyl)phenyl)benzo[d]imidazo[2,1- |
| | b]thiazole-7-carboxamide |
| 345 | N-(3-(diethylamino)propyl)-2-(4-(morpholinomethyl)phenyl)benzo[d]imidazo[2,1- |
| | b]thiazole-7-carboxamide |
| 346 | N-(3-(diethylamino)propyl)-2-(4-(piperazin-1-ylmethyl)phenyl)benzo[d]imidazo[2,1- |
| | b]thiazole-7-carboxamide |
| 348 | 2-(4-(aminomethyl)phenyl)-N-(3-(diethylamino)propyl)benzo[4,5]thiazolo[3,2- |
| | b][1,2,4]triazole-6-carboxamide |
| 351 | 2-(4-(aminomethyl)phenyl)-N-(3-(diethylamino)propyl)benzo[4,5]imidazo[2,1- |
| | b]thiazole-7-carboxamide |
| 354 | 2-(4-(aminomethyl)phenyl)-N-(3- |
| | (diethylamino)propyl)imidazo[2′,1′:2,3]thiazolo[4,5-b]pyridine-7-carboxamide |
| 356 | 2-(4-(aminomethyl)phenyl)-N-(3- |
| | (diethylamino)propyl)imidazo[2′,1′:2,3]thiazolo[5,4-b]pyridine-7-carboxamide |
| 359 | 7-(4-(aminomethyl)phenyl)-N-(3- |
| | (diethylamino)propyl)imidazo[2′,1′:2,3]thiazolo[5,4-d]pyrimidine-2-carboxamide |
| 368 | 2-(4-((cyclopropylamino)methyl)-2-fluorophenyl)-N-(3-(piperidin-1- |
| | yl)propyl)benzo[d]imidazo[2,1-b]thiazole-7-carboxamide |
| 369 | 2-(4-(aminomethyl)-2-chlorophenyl)-N-(3-(piperidin-1- |
| | yl)propyl)benzo[d]imidazo[2,1-b]thiazole-7-carboxamide |
| 370 | 2-(4-(aminomethyl)-2-cyclopropylphenyl)-N-(3-(piperidin-1- |
| | yl)propyl)benzo[d]imidazo[2,1-b]thiazole-7-carboxamide |
| 371 | 2-(4-(aminomethyl)-2-(difluoromethyl)phenyl)-N-(3-(piperidin-1- |
| | yl)propyl)benzo[d]imidazo[2,1-b]thiazole-7-carboxamide |
| 372 | 2-(4-(aminomethyl)-2-(trifluoromethyl)phenyl)-N-(3-(piperidin-1- |
| | yl)propyl)benzo[d]imidazo[2,1-b]thiazole-7-carboxamide |
| 373 | N-(3-(piperidin-1-yl)propyl)-2-(4-(pyrrolidin-2-yl)phenyl)benzo[d]imidazo[2,1- |
| | b]thiazole-7-carboxamide |
| 374 | 2-(4-(aminomethyl)-2-fluorophenyl)-N-(3-(piperidin-1- |
| | yl)propyl)benzo[d]imidazo[2,1-b]thiazole-7-carboxamide |
| 375 | 2-(4-(aminomethyl)-3-fluorophenyl)-N-(3-(piperidin-1- |
| | yl)propyl)benzo[d]imidazo[2,1-b]thiazole-7-carboxamide |
| 376 | 2-(4-(aminomethyl)-3-chlorophenyl)-N-(3-(piperidin-1- |
| | yl)propyl)benzo[d]imidazo[2,1-b]thiazole-7-carboxamide |
| 377 | 2-(4-(aminomethyl)-3-cyclopropylphenyl)-N-(3-(piperidin-1- |
| | yl)propyl)benzo[d]imidazo[2,1-b]thiazole-7-carboxamide |
| 378 | 2-(4-(aminomethyl)-3-(trifluoromethyl)phenyl)-N-(3-(piperidin-1- |
| | yl)propyl)benzo[d]imidazo[2,1-b]thiazole-7-carboxamide |
| 379 | 2-(4-(aminomethyl)-3-(difluoromethyl)phenyl)-N-(3-(piperidin-1- |
| | yl)propyl)benzo[d]imidazo[2,1-b]thiazole-7-carboxamide |
| 380 | 2-(4-(aminomethyl)-3-isopropylphenyl)-N-(3-(piperidin-1- |
| | yl)propyl)benzo[d]imidazo[2,1-b]thiazole-7-carboxamide |
| 381 | 2-(4-(aminomethyl)-3-methylphenyl)-N-(3-(piperidin-1- |
| | yl)propyl)benzo[d]imidazo[2,1-b]thiazole-7-carboxamide |
| 382 | 2-(4-(aminomethyl)-3,5-dimethylphenyl)-N-(3-(piperidin-1- |
| | yl)propyl)benzo[d]imidazo[2,1-b]thiazole-7-carboxamide |
| 383 | 2-(4-(aminomethyl)-3,5-difluorophenyl)-N-(3-(piperidin-1- |
| | yl)propyl)benzo[d]imidazo[2,1-b]thiazole-7-carboxamide |
| 385 | 2-(4-(aminomethyl)-3,5-diisopropylphenyl)-N-(3-(piperidin-1- |
| | yl)propyl)benzo[d]imidazo[2,1-b]thiazole-7-carboxamide |
| 387 | 2-(4-(aminomethyl)-2-fluorophenyl)-N-(3-(piperidin-1- |
| | yl)propyl)benzo[4,5]thiazolo[3,2-b][1,2,4]triazole-6-carboxamide |
| 388 | 2-(4-(aminomethyl)-2-fluorophenyl)-N-(3- |
| | (diethylamino)propyl)benzo[4,5]thiazolo[3,2-b][1,2,4]triazole-6-carboxamide |
| 390 | 2-(4-(aminomethyl)-2-fluorophenyl)-N-(3-(4-fluoropiperidin-1- |
| | yl)propyl)benzo[4,5]thiazolo[3,2-b][1,2,4]triazole-6-carboxamide |
| 392 | 2-(4-(aminomethyl)phenyl)-N-(3-(piperidin-1-yl)propyl)benzo[4,5]thiazolo[3,2- |
| | b][1,2,4]triazole-6-carboxamide |
| 393 | 2-(4-((cyclopropylamino)methyl)phenyl)-N-(3-(piperidin-1- |
| | yl)propyl)benzo[4,5]thiazolo[3,2-b][1,2,4]triazole-6-carboxamide |
| 394 | 2-(4-((cyclopropylamino)methyl)phenyl)-N-(3- |
| | (diethylamino)propyl)benzo[4,5]thiazolo[3,2-b][1,2,4]triazole-6-carboxamide |
| 395 | 2-(4-((cyclopropylamino)methyl)-2-fluorophenyl)-N-(3- |
| | (diethylamino)propyl)benzo[4,5]thiazolo[3,2-b][1,2,4]triazole-6-carboxamide |
| 396 | 2-(4-((cyclopropylamino)methyl)-2-fluorophenyl)-N-(3-(piperidin-1- |
| | yl)propyl)benzo[4,5]thiazolo[3,2-b][1,2,4]triazole-6-carboxamide |
| 397 | 2-(4-(1-aminocyclopropyl)phenyl)-N-(3- |
| | (diethylamino)propyl)benzo[4,5]thiazolo[3,2-b][1,2,4]triazole-6-carboxamide |
| 398 | 2-(4-(1-aminocyclopropyl)phenyl)-N-(3-(piperidin-1- |
| | yl)propyl)benzo[4,5]thiazolo[3,2-b][1,2,4]triazole-6-carboxamide |
| 399 | 2-(4-(1-aminocyclopropyl)-2-fluorophenyl)-N-(3- |
| | (diethylamino)propyl)benzo[4,5]thiazolo[3,2-b][1,2,4]triazole-6-carboxamide |
| 401 | 2-(4-(aminomethyl)phenyl)-N-(3- |
| | (diethylamino)propyl)imidazo[2′,1′:2,3[thiazolo[4,5-c]pyridine-7-carboxamide |
| 402 | 2-(4-((cyclopropylamino)methyl)phenyl)-N-(3- |
| | (diethylamino)propyl)imidazo[2′,1′:2,3]thiazolo[4,5-c]pyridine-7-carboxamide |
| 404 | 2-(4-(aminomethyl)-2-fluorophenyl)-N-(3- |
| | (diethylamino)propyl)imidazo[2′,1′:2,3]thiazolo[4,5-c]pyridine-7-carboxamide |
| 405 | 2-(4-((cyclopropylamino)methyl)-2-fluorophenyl)-N-(3- |
| | (diethylamino)propyl)imidazo[2′,1′:2,3]thiazolo[4,5-c]pyridine-7-carboxamide |
| 407 | 2-(4-(aminomethyl)phenyl)-N-(3-(piperidin-1- |
| | yl)propyl)imidazo[2′,1′:2,3]thiazolo[4,5-c]pyridine-7-carboxamide |
| 408 | 2-(4-((cyclopropylamino)methyl)phenyl)-N-(3-(piperidin-1- |
| | yl)propyl)imidazo[2′,1′:2,3]thiazolo[4,5-c]pyridine-7-carboxamide |
| 410 | 2-(4-(aminomethyl)-2-fluorophenyl)-N-(3-(piperidin-1- |
| | yl)propyl)imidazo[2′,1′:2,3]thiazolo[4,5-c]pyridine-7-carboxamide |
| 411 | 2-(4-((cyclopropylamino)methyl)-2-fluorophenyl)-N-(3-(piperidin-1- |
| | yl)propyl)imidazo[2′,1′:2,3]thiazolo[4,5-c]pyridine-7-carboxamide |
| 412 | (R)-N-(3-(piperidin-1-yl)propyl)-2-(4-(pyrrolidin-2-yl)phenyl)benzo[d]imidazo[2,1- |
| | b]thiazole-7-carboxamide |
| 413 | (S)-N-(3-(piperidin-1-yl)propyl)-2-(4-(pyrrolidin-2-yl)phenyl)benzo[d]imidazo[2,1- |
| | b]thiazole-7-carboxamide |
| 414 | (R)-N-(3-(diethylamino)propyl)-2-(4-(pyrrolidin-2-yl)phenyl)benzo[d]imidazo[2,1- |
| | b]thiazole-7-carboxamide |
| 415 | (S)-N-(3-(diethylamino)propyl)-2-(4-(pyrrolidin-2-yl)phenyl)benzo[d]imidazo[2,1- |
| | b]thiazole-7-carboxamide |
| 416 | 2-(2-fluoro-4-(pyrrolidin-2-yl)phenyl)-N-(3-(piperidin-1- |
| | yl)propyl)benzo[d]imidazo[2,1-b]thiazole-7-carboxamide |
| 417 | (R)-2-(2-fluoro-4-(pyrrolidin-2-yl)phenyl)-N-(3-(piperidin-1- |
| | yl)propyl)benzo[d]imidazo[2,1-b]thiazole-7-carboxamide |
| 418 | (S)-2-(2-fluoro-4-(pyrrolidin-2-yl)phenyl)-N-(3-(piperidin-1- |
| | yl)propyl)benzo[d]imidazo[2,1-b]thiazole-7-carboxamide |
| 419 | (R)-2-(2-fluoro-4-(pyrrolidin-2-yl)phenyl)-N-(3-(4-fluoropiperidin-1- |
| | yl)propyl)benzo[d]imidazo[2,1-b]thiazole-7-carboxamide |
| 420 | (S)-2-(2-fluoro-4-(pyrrolidin-2-yl)phenyl)-N-(3-(4-fluoropiperidin-1- |
| | yl)propyl)benzo[d]imidazo[2,1-b]thiazole-7-carboxamide |
| 421 | (R)-2-(2-fluoro-4-(pyrrolidin-2-yl)phenyl)-N-(3-(piperidin-1- |
| | yl)propyl)benzo[4,5]thiazolo[3,2-b][1,2,4]triazole-6-carboxamide |
| 422 | (S)-2-(2-fluoro-4-(pyrrolidin-2-yl)phenyl)-N-(3-(piperidin-1- |
| | yl)propyl)benzo[4,5]thiazolo[3,2-b][1,2,4]triazole-6-carboxamide |
| 423S | (S)-2-(2-fluoro-4-(pyrrolidin-2-yl)phenyl)-N-(3-(2-oxopiperidin-1- |
| | yl)propyl)benzo[d]imidazo[2,1-b]thiazole-7-carboxamide |
| 423R | (R)-2-(2-fluoro-4-(pyrrolidin-2-yl)phenyl)-N-(3-(2-oxopiperidin-1- |
| | yl)propyl)benzo[d]imidazo[2,1-b]thiazole-7-carboxamide |
| 424 | N-(3-(4-cyanopiperidin-1-yl)propyl)-2-(2-fluoro-4-(pyrrolidin-2- |
| | yl)phenyl)benzo[d]imidazo[2,1-b]thiazole-7-carboxamide |
| 426 | 2-(2-fluoro-4-(pyrrolidin-2-yl)phenyl)-N-(3-(4-(trifluoromethyl)piperidin-1- |
| | yl)propyl)benzo[d]imidazo[2,1-b]thiazole-7-carboxamide |
| 427S | (S)-N-(3-(4-fluoropiperidin-1-yl)propyl)-2-(4-(pyrrolidin-2-yl)-3- |
| | (trifluoromethyl)phenyl)benzo[d]imidazo[2,1-b]thiazole-7-carboxamide |
| 427R | (R)-N-(3-(4-fluoropiperidin-1-yl)propyl)-2-(4-(pyrrolidin-2-yl)-3- |
| | (trifluoromethyl)phenyl)benzo[d]imidazo[2,1-b]thiazole-7-carboxamide |
| 429S | N-(3-(4-fluoro-2-methylpiperidin-1-yl)propyl)-2-(2-fluoro-4-((S)-pyrrolidin-2- |
| | yl)phenyl)benzo[d]imidazo[2,1-b]thiazole-7-carboxamide |
| 429R | N-(3-(4-fluoro-2-methylpiperidin-1-yl)propyl)-2-(2-fluoro-4-((R)-pyrrolidin-2- |
| | yl)phenyl)benzo[d]imidazo[2,1-b]thiazole-7-carboxamide |
| 430S | N-(3-(4-fluoro-2-oxopiperidin-1-yl)propyl)-2-(2-fluoro-4-((S)-pyrrolidin-2- |
| | yl)phenyl)benzo[d]imidazo[2,1-b]thiazole-7-carboxamide |
| 430R | N-(3-(4-fluoro-2-oxopiperidin-1-yl)propyl)-2-(2-fluoro-4-((R)-pyrrolidin-2- |
| | yl)phenyl)benzo[d]imidazo[2,1-b]thiazole-7-carboxamide |
| 431S | N-(3-(6-fluoro-3-azabicyclo[3.1.1]heptan-3-yl)propyl)-2-(2-fluoro-4-((S)pyrrolidin- |
| | 2-yl)phenyl)benzo[d]imidazo[2,1-b]thiazole-7-carboxamide |
| 431R | N-(3-(6-fluoro-3-azabicyclo[3.1.1]heptan-3-yl)propyl)-2-(2-fluoro-4-((R)-pyrrolidin- |
| | 2-yl)phenyl)benzo[d]imidazo[2,1-b]thiazole-7-carboxamide |
| 432S | (S)-2-(2-fluoro-4-(5-oxopyrrolidin-2-yl)phenyl)-N-(3-(4-fluoropiperidin-1- |
| | yl)propyl)benzo[d]imidazo[2,1-b]thiazole-7-carboxamide |
| 432R | (R)-2-(2-fluoro-4-(5-oxopyrrolidin-2-yl)phenyl)-N-(3-(4-fluoropiperidin-1- |
| | yl)propyl)benzo[d]imidazo[2,1-b]thiazole-7-carboxamide |
| 433S | (S)-2-(2-fluoro-4-(1-methylpyrrolidin-2-yl)phenyl)-N-(3-(4-fluoropiperidin-1- |
| | yl)propyl)benzo[d]imidazo[2,1-b]thiazole-7-carboxamide |
| 433R | (R)-2-(2-fluoro-4-(1-methylpyrrolidin-2-yl)phenyl)-N-(3-(4-fluoropiperidin-1- |
| | yl)propyl)benzo[d]imidazo[2,1-b]thiazole-7-carboxamide |
| 436S | (S)-2-(2-fluoro-4-(piperidin-2-yl)phenyl)-N-(3-(4-fluoropiperidin-1- |
| | yl)propyl)benzo[d]imidazo[2,1-b]thiazole-7-carboxamide |
| 436R | (R)-2-(2-fluoro-4-(piperidin-2-yl)phenyl)-N-(3-(4-fluoropiperidin-1- |
| | yl)propyl)benzo[d]imidazo[2,1-b]thiazole-7-carboxamide |
| 442 | (S)-3-((cyclopropylmethyl)amino)-N-(3-(4-fluoropiperidin-1-yl)propyl)-2-(4- |
| | (pyrrolidin-2-yl)phenyl)benzo[d]imidazo[2,1-b]thiazole-7-carboxamide |
| 443 | (R)-3-((cyclopropylmethyl)amino)-N-(3-(4-fluoropiperidin-1-yl)propyl)-2-(4- |
| | (pyrrolidin-2-yl)phenyl)benzo[d]imidazo[2,1-b]thiazole-7-carboxamide |
| 444 | (S)-3-((cyclopropylmethyl)amino)-2-(2-fluoro-4-(pyrrolidin-2-yl)phenyl)-N-(3-(4- |
| | fluoropiperidin-1-yl)propyl)benzo[d]imidazo[2,1-b]thiazole-7-carboxamide |
| 445 | (R)-3-((cyclopropylmethyl)amino)-2-(2-fluoro-4-(pyrrolidin-2-yl)phenyl)-N-(3-(4- |
| | fluoropiperidin-1-yl)propyl)benzo[d]imidazo[2,1-b]thiazole-7-carboxamide |
| 448 | N-(3-(4-fluoro-2-oxopiperidin-1-yl)propyl)-2-(2-fluoro-4-((S)-5-oxopyrrolidin-2- |
| | yl)phenyl)benzo[d]imidazo[2,1-b]thiazole-7-carboxamide |
| 449 | N-(3-(4-fluoro-2-oxopiperidin-1-yl)propyl)-2-(2-fluoro-4-((R)-5-oxopyrrolidin-2- |
| | yl)phenyl)benzo[d]imidazo[2,1-b]thiazole-7-carboxamide |
| 450S | (S)-2-(2,6-difluoro-4-(pyrrolidin-2-yl)phenyl)-N-(3-(4-fluoropiperidin-1- |
| | yl)propyl)benzo[d]imidazo[2,1-b]thiazole-7-carboxamide |
| 450R | (R)-2-(2,6-difluoro-4-(pyrrolidin-2-yl)phenyl)-N-(3-(4-fluoropiperidin-1- |
| | yl)propyl)benzo[d]imidazo[2,1-b]thiazole-7-carboxamide |
| 451S | (S)-2-(2,6-dimethyl-4-(pyrrolidin-2-yl)phenyl)-N-(3-(4-fluoropiperidin-1- |
| | yl)propyl)benzo[d]imidazo[2,1-b]thiazole-7-carboxamide |
| 451R | (R)-2-(2,6-dimethyl-4-(pyrrolidin-2-yl)phenyl)-N-(3-(4-fluoropiperidin-1- |
| | yl)propyl)benzo[d]imidazo[2,1-b]thiazole-7-carboxamide |
| 452S | (S)-2-(2-cyclopropyl-4-(pyrrolidin-2-yl)phenyl)-N-(3-(4-fluoropiperidin-1- |
| | yl)propyl)benzo[d]imidazo[2,1-b]thiazole-7-carboxamide |
| 452R | (R)-2-(2-cyclopropyl-4-(pyrrolidin-2-yl)phenyl)-N-(3-(4-fluoropiperidin-1- |
| | yl)propyl)benzo[d]imidazo[2,1-b]thiazole-7-carboxamide |
| 453S | (S)-2-(2,3-dimethyl-4-(pyrrolidin-2-yl)phenyl)-N-(3-(4-fluoropiperidin-1- |
| | yl)propyl)benzo[d]imidazo[2,1-b]thiazole-7-carboxamide |
| 453R | (R)-2-(2,3-dimethyl-4-(pyrrolidin-2-yl)phenyl)-N-(3-(4-fluoropiperidin-1- |
| | yl)propyl)benzo[d]imidazo[2,1-b]thiazole-7-carboxamide |
| 462S | (S)-2-(2-fluoro-4-(pyrrolidin-2-yl)phenyl)-N-(3-(4-fluoropiperidin-1-yl)propyl)-3- |
| | (hydroxymethyl)benzo[d]imidazo[2,1-b]thiazole-7-carboxamide |
| 462R | (R)-2-(2-fluoro-4-(pyrrolidin-2-yl)phenyl)-N-(3-(4-fluoropiperidin-1-yl)propyl)-3- |
| | (hydroxymethyl)benzo[d]imidazo[2,1-b]thiazole-7-carboxamide |
| 463S | (S)-2-(2-fluoro-4-(pyrrolidin-2-yl)phenyl)-N-(3-(4-fluoropiperidin-1-yl)propyl)-3-(2- |
| | hydroxyethyl)benzo[d]imidazo[2,1-b]thiazole-7-carboxamide |
| 463R | (R)-2-(2-fluoro-4-(pyrrolidin-2-yl)phenyl)-N-(3-(4-fluoropiperidin-1-yl)propyl)-3-(2- |
| | hydroxyethyl)benzo[d]imidazo[2,1-b]thiazole-7-carboxamide |
| 464S | (S)-2-(2-fluoro-4-(pyrrolidin-2-yl)phenyl)-N-(3-(4-fluoropiperidin-1-yl)propyl)-3- |
| | methylbenzo[d]imidazo[2,1-b]thiazole-7-carboxamide |
| 464R | (R)-2-(2-fluoro-4-(pyrrolidin-2-yl)phenyl)-N-(3-(4-fluoropiperidin-1-yl)propyl)-3- |
| | methylbenzo[d]imidazo[2,1-b]thiazole-7-carboxamide |
| 467 | (S)-3-cyclopropyl-N-(3-(4-fluoropiperidin-1-yl)propyl)-2-(4-(pyrrolidin-2- |
| | yl)phenyl)benzo[d]imidazo[2,1-b]thiazole-7-carboxamide |
| 468 | (R)-3-cyclopropyl-N-(3-(4-fluoropiperidin-1-yl)propyl)-2-(4-(pyrrolidin-2- |
| | yl)phenyl)benzo[d]imidazo[2,1-b]thiazole-7-carboxamide |
| 469S | (S)-2-(2-fluoro-4-(pyrrolidin-2-yl)phenyl)-N-(3-(4-fluoropiperidin-1-yl)propyl)-3- |
| | isopropoxybenzo[d]imidazo[2,1-b]thiazole-7-carboxamide |
| 469R | (R)-2-(2-fluoro-4-(pyrrolidin-2-yl)phenyl)-N-(3-(4-fluoropiperidin-1-yl)propyl)-3- |
| | isopropoxybenzo[d]imidazo[2,1-b]thiazole-7-carboxamide |
| 473S | (S)-2-(2-fluoro-4-(pyrrolidin-2-yl)phenyl)-7-((3-(4-fluoropiperidin-1- |
| | yl)propyl)carbamoyl)benzo[d]imidazo[2,1-b]thiazole-3-carboxylic acid |
| 473R | (R)-2-(2-fluoro-4-(pyrrolidin-2-yl)phenyl)-7-((3-(4-fluoropiperidin-1- |
| | yl)propyl)carbamoyl)benzo[d]imidazo[2,1-b]thiazole-3-carboxylic acid |

4. A compound represented by the structure of formula (I): 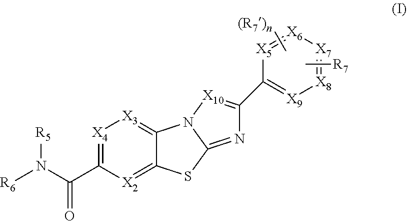
wherein
X₂, X₃, and X₄, are each independently nitrogen or CH;
X₅, X₆, X₇, X₈ and X₉ are each independently nitrogen or carbon atoms;
X₁₀ is N, CH, or C(R);
R₅ is H or C₁-C₅ linear or branched alkyl;
R is H, F, Cl, Br, I, OH, SH, OH, alkoxy, NH(R₁₀), NH—CH₂-cyclopropyl, N(R₁₀)(R₁₁), CF₃, CN, NO₂, COOH, C₁-C₅ linear or branched, substituted or unsubstituted alkyl, methyl, ethyl, CH₂—OH, CH₂—CH₂—OH, C₃-C₈ substituted or unsubstituted cycloalkyl, cyclopropyl, C₁-C₅ linear or branched alkoxy, isopropoxy, C₁-C₅ linear or branched haloalkyl, R₈-aryl, —R₈—O—R₈—O—R₁₀, —R₈—O—R₁₀, —R₈-R₁₀, substituted or unsubstituted aryl, substituted or unsubstituted heteroaryl;
each R₈ is independently [CH₂]ₚ
wherein p is between 1 and 10;
R₉ is [CH]_{q}, [C]_{q}
wherein q is between 2 and 10;
R₁₀ and Ru are each independently H, C₁-C₅ substituted or unsubstituted linear or branched alkyl, methyl, CH₂-cyclopropyl, C₁-C₅ substituted or unsubstituted linear or branched haloalky, CH₂CF₃, C₁-C₅ linear or branched alkoxy, R₂₀, C(O)R, or S(O)₂R;
or R₁₀ and Ru are joined to form a substituted or unsubstituted 3-8 membered heterocyclic ring;
R₂₀ is represented by the following structure: 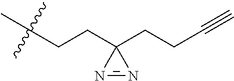 [SMILES: *CCC1(CCC#C)N=N1]
n is an integer between 0 and 4 (e.g., 1, 2);
AND
EITHER ONE OF ALTERNATIVES 1-5 TAKES PLACE:
1.
R₆ is H, F, Cl, Br, I, OH, SH, R₈—OH, R₈—SH, —R₈—O—R₁₀, R₈—S—R₁₀, (CH₂)₃—S—(CH₂)₂CH₃), R₈—NHC(O)—R₁₀, —O—R₈—R₁₀, R₈-(substituted or unsubstituted C₃-C₈ cycloalkyl), R₈-(substituted or unsubstituted saturated, unsaturated or aromatic, single, fused or spiro 3-10 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), (CH₂)₃—N(CH₂CH₃)₂, (CH₂)₃—NH₂, (CH₂)₃—N(CH₂CH₃)(CH₂CF₃, R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, R₈-(substituted or unsubstituted C₃-C₈ cycloalkyl), substituted or unsubstituted 3-8 membered heterocyclic ring, substituted or unsubstituted aryl, substituted or unsubstituted R₈-aryl, or substituted or unsubstituted benzyl;
or R₆ is represented by the structure of formula Bi: 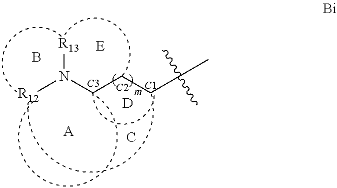
wherein
m is 0 or 1; and
R₁₂ is R₂₀ or C₁-C₅ C(O)-alkyl, and R₁₃ is R₃₀; or
R₁₂ and R₁₃ are both H; or
R₁₂ and R₁₃ are each independently H or substituted or unsubstituted C₁-C₅ alkyl; or
R₁₂ and C3 are joined to form ring A and R₁₃ is R₃₀; or
R₁₂ and R₁₃ are joined to form ring B; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
Ring A, C and E are each independently a substituted or unsubstituted single, spiro or fused 3-8 membered heterocyclic ring;
Ring B is a substituted or unsubstituted single, spiro or fused 3-8 membered heterocyclic ring; and
Ring D is a substituted or unsubstituted C₃-C₈ cycloalkyl;
or R₆ and R₅ are joined to for a substituted or unsubstituted 5-8 membered heterocyclic ring;
R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl, C₁-C₅ linear or branched alkoxy, C₁-C₅ linear or branched haloalkyl, R₈-aryl, —R₈—O—R₈—O—R₁₀, —R₈—O—R₁₀, —R₈-R₁₀, substituted or unsubstituted aryl, substituted or unsubstituted heteroaryl;
AND
R₇ is O—R₂₀, substituted or unsubstituted 4-7 membered heterocyclic ring (pyrrolidine, pyrrolidinone, morpholine, oxetane, imidazole, tetrahydropyran, triazole, oxadiazole, pyrazole), substituted or unsubstituted aryl, R₈-(substituted or unsubstituted, saturated, unsaturated or aromatic, single, fused or spiro 3-8 membered heterocyclic ring);
or R₇ is represented by the structure of formula A: 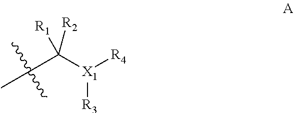
wherein
X₁ is N or 0;
R₁ and R₂ are each independently H, F, or CF₃; or R₁ and R₂ are joined to form a C₃-C₈ carbocyclic (e.g., cyclopropyl) or heterocyclic ring; and
R₃ and R₄ are each independently H, Me, substituted or unsubstituted C₁-C₅ alkyl, substituted or unsubstituted C₃-C₈ cycloalkyl, cyclopropyl, substituted or unsubstituted 5-7 membered heterocyclic ring, or R₂₀;
or R₃ and R₄ are joined to form a 3-8 membered heterocyclic ring;
wherein if X₁ is O then R₄ is absent;
AND
R₇′ is H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OCH₂Ph, COOH, C(O)H, —C(O)NH₂, SO₂R, SO₂N(R₁₀)(R₁₁), CH(CF₃)(NH—R₁₀), C₁-C₅ linear or branched, substituted or unsubstituted alkyl, methyl, C₁-C₅ linear or branched, substituted or unsubstituted alkenyl, C₁-C₅ linear or branched, or C₃-C₈ cyclic haloalkyl, CHF₂, C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, cyclopropyl, substituted or unsubstituted 3-8 membered heterocyclic ring, substituted or unsubstituted aryl, substituted or unsubstituted benzyl;
or R₇ and R₇′ are joined to form a 5 or 6 membered substituted or unsubstituted, saturated, unsaturated or aromatic, carbocyclic or heterocyclic ring, piperidine, pyrrolidine, tetrahydrofuran, or tetrahydropyran;
OR
2.
R₆ is —R₈—O—R₁₀, R₈—S—R₁₀, R₈—NH₂, R₈—NHC(O)—R₁₀, R₈—C(O)N(R₁₀)(R₁₁), R₈-(substituted or unsubstituted C₃-C₈ cycloalkyl), (CH₂)₃-pyran, CH₂-tetrahydrofurane, CH₂-dioxane, CH₂-methyl-THF, CH₂-tetrahydrofurane, CH₂-oxa-azaspirodecane, CH₂-azaspiroheptane, (CH₂)₃-dimethylpyrazole, CH₂-methyl-azetidine, CH₂-azaspiroheptane, CH₂-pyrrolidine, (CH₂)₃-pyrrolidine, which may all be substituted or unsubstituted, benzyl, C₁-C₅ linear or branched alkoxyalkyl, substituted or unsubstituted C₃-C₈ cycloalkyl;
or R₆ is represented by the structure of formula Bi: 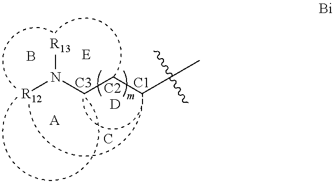
wherein
m is 0 or 1; and
R₁₂ is R₂₀ or C₁-C₅ C(O)-alkyl, and R₁₃ is R₃₀; or
R₁₂ and R₁₃ are both H; or
R₁₂ and C3 are joined to form ring A and R₁₃ is R₃₀; or
R₁₂ and R₁₃ are joined to form a substituted or unsubstituted piperidin-2-one, 4-fluoropiperidin-2-one, piperidine-4-carbonitrile, 4-fluoropiperidine, 4-fluoro-2-methylpiperidine, pyrrolidine ring, piperazine, thiomorpholine 1,1-dioxide, 2-oxa-6-azaspiro[3.3]heptane, pyrazole, imidazole, 2,5-diazabicyclo[2.2.1]heptane diazabicyclo[2.2.1]heptane, or a 6-fluoro-3-azabicyclo[3.1.1]heptane; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
Ring A, C and E are each independently a substituted or unsubstituted single, spiro or fused 3-8 membered heterocyclic ring;
Ring B is a substituted or unsubstituted single, spiro or fused 3-8 membered heterocyclic ring; and
Ring D is a substituted or unsubstituted C₃-C₈ cycloalkyl;
R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl, C₁-C₅ linear or branched alkoxy, C₁-C₅ linear or branched haloalkyl, R₈-aryl, —R₈—O—R₈—O—R₁₀, —R₈—O—R₁₀, —R₈-R₁₀, substituted or unsubstituted aryl, substituted or unsubstituted heteroaryl;
AND
R₇ is F, Cl, Br, I, OH, O—R₂₀, SH, R₈—OH, R₈—SH, SR₁₀, —R₈—O—R₁₀, —R₈—S—R₁₀, R₈—(C₃-C₈ cycloalkyl), R₈-(substituted or unsubstituted saturated, unsaturated or aromatic, single, fused or spiro 3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OCH₂Ph, COOH, C(O)H, —C(O)NH₂, SO₂R, SO₂N(R₁₀)(R₁₁), CH(CF₃)(NH—R₁₀), C₁-C₅ linear or branched, substituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, cyclopropanol, cyclohexyl, substituted or unsubstituted 4-7 membered heterocyclic ring, tetrahydrofuran, oxetane, oxetan-3-ol, pyrrolidine, 1-methylpyrrolidine, pyrrolidin-2-one, piperidine, piperidine-4-carbonitrile, 4-fluoropiperidine, substituted or unsubstituted aryl, substituted or unsubstituted benzyl;
or R₇ is represented by the structure of formula A: 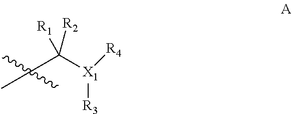
wherein
X₁ is N or O;
R₁ and R₂ are each independently H, F, or CF₃; or R₁ and R₂ are joined to form a C₃-C₈ carbocyclic or heterocyclic ring;
R₃ and R₄ are each independently H, Me, substituted or unsubstituted C₁-C₅ alkyl, substituted or unsubstituted C₃-C₈ cycloalkyl, substituted or unsubstituted 5-6 membered heterocyclic ring, or R₂₀; or R₃ and R₄ are joined to form a 3-8 membered heterocyclic ring;
wherein if X₁ is O then R₄ is absent;
AND
R₇′ is H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OCH₂Ph, COOH, C(O)H, —C(O)NH₂, SO₂R, SO₂N(R₁₀)(R₁₁), CH(CF₃)(NH—R₁₀), C₁-C₅ linear or branched, substituted or unsubstituted alkyl, methyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, cyclopropyl, substituted or unsubstituted 3-8 membered heterocyclic ring, substituted or unsubstituted aryl, substituted or unsubstituted benzyl;
or R₇ and R₇′ are joined to form a 5 or 6 membered substituted or unsubstituted, saturated, unsaturated or aromatic, carbocyclic or heterocyclic ring, piperidine, pyrrolidine, tetrahydrofuran, or tetrahydropyran;
OR
3.
R₆ is H, F, Cl, Br, I, OH, SH, R₈—OH, R₈—SH, —R₈—O—R₁₀, R₈—S—R₁₀, (CH₂)₃—S—(CH₂)₂CH₃), R₈—NHC(O)—R₁₀, —O—R₈—R₁₀, R₈-(substituted or unsubstituted C₃-C₈ cycloalkyl), R₈-(substituted or unsubstituted saturated, unsaturated or aromatic, single, fused or spiro 3-10 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), (CH₂)₃—N(CH₂CH₃)₂, (CH₂)₃—NH₂, (CH₂)₃—N(CH₂CH₃)(CH₂CF₃, R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, R₈-(substituted or unsubstituted C₃-C₈ cycloalkyl), substituted or unsubstituted 3-8 membered heterocyclic ring, substituted or unsubstituted aryl, substituted or unsubstituted R₈-aryl, or substituted or unsubstituted benzyl;
or R₆ is represented by the structure of formula Bi: 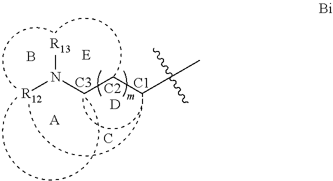
wherein
m is 0 or 1; and
R₁₂ is C20 or C₁-C₅ C(O)-alkyl, and R₁₃ is R₃₀; or
R₁₂ and R₁₃ are both H; or
R₁₂ and R₁₃ are each independently H or substituted or unsubstituted C₁-C₅ alkyl; or
R₁₂ and C3 are joined to form ring A and R₁₃ is R₃₀; or
R₁₂ and R₁₃ are joined to form ring B; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
Ring A, C and E are each independently a substituted or unsubstituted single, spiro or fused 3-8 membered heterocyclic ring;
Ring B is a substituted or unsubstituted single, spiro or fused 3-8 membered heterocyclic ring; and
Ring D is a substituted or unsubstituted C₃-C₈ cycloalkyl;
or R₆ and R₅ are joined to for a substituted or unsubstituted 5-8 membered heterocyclic ring;
R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl, C₁-C₅ linear or branched alkoxy, C₁-C₅ linear or branched haloalkyl, R₈-aryl, —R₈—O—R₈—O—R₁₀, —R₈—O—R₁₀, —R₈-R₁₀, substituted or unsubstituted aryl, substituted or unsubstituted heteroaryl;
AND
R₇ is Br, I, OH, O—R₂₀, SH, R₈—OH, R₈—SH, SR₁₀, —R₈—O—R₁₀, —R₈—S—R₁₀, R₈—(C₃-C₈ cycloalkyl), R₈-(substituted or unsubstituted single, fused or spiro 3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OCH₂Ph, COOH, C(O)H, —C(O)NH₂, SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched thioalkyl, C₁-C₅ linear or branched thioalkoxy, C₁-C₅ linear or branched alkoxyalkyl, substituted or unsubstituted C₃-C₈ cycloalkyl, cyclopropanol, cyclohexyl, substituted or unsubstituted 4-7 membered heterocyclic ring, tetrahydrofuran, oxetane, oxetan-3-ol, pyrrolidine, 1-methylpyrrolidine, pyrrolidin-2-one, piperidine, piperidine-4-carbonitrile, 4-fluoropiperidine, substituted or unsubstituted aryl, substituted or unsubstituted benzyl;
or R₇ is represented by the structure of formula A: 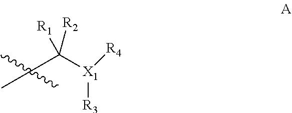
wherein
X₁ is N or 0;
R₁ and R₂ are each independently H, F, or CF₃; or R₁ and R₂ are joined to form a C₃-C₈ carbocyclic or heterocyclic ring;
R₃ and R₄ are each independently H, Me, substituted or unsubstituted C₁-C₅ alkyl, substituted or unsubstituted C₃-C₈ cycloalkyl, substituted or unsubstituted 5-7 membered heterocyclic ring, or R₂₀; or R₃ and R₄ are joined to form a 3-8 membered heterocyclic ring;
wherein if X₁ is O then R₄ is absent;
AND
R₇′ is F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OCH₂Ph, COOH, C(O)H, —C(O)NH₂, SO₂R, SO₂N(R₁₀)(Rn), CH(CF₃)(NH—R₁₀), C₁-C₅ linear or branched, substituted or unsubstituted alkyl, methyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, cyclopropyl, substituted or unsubstituted 3-8 membered heterocyclic ring, substituted or unsubstituted aryl, substituted or unsubstituted benzyl;
or R₇ and R₇′ are joined to form a 5 or 6 membered substituted or unsubstituted, saturated, unsaturated or aromatic, carbocyclic or heterocyclic ring, piperidine, pyrrolidine, tetrahydrofuran, or tetrahydropyran;
wherein R₇′ is different than R₇; and
wherein n is not 0;
OR
4.
R₆ is H, F, Cl, Br, I, OH, SH, R₈—OH, R₈—SH, —R₈—O—R₁₀, R₈—S—R₁₀, (CH₂)₃—S—(CH₂)₂CH₃), R₈—NHC(O)—R₁₀, —O—R₈—R₁₀, R₈-(substituted or unsubstituted C₃-C₈ cycloalkyl), R₈-(substituted or unsubstituted saturated, unsaturated or aromatic, single, fused or spiro 3-10 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), (CH₂)₃—N(CH₂CH₃)₂, (CH₂)₃—NH₂, (CH₂)₃—N(CH₂CH₃)(CH₂CF₃, R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, R₈-(substituted or unsubstituted C₃-C₈ cycloalkyl), substituted or unsubstituted 3-8 membered heterocyclic ring, substituted or unsubstituted aryl, substituted or unsubstituted R₈-aryl, or substituted or unsubstituted benzyl;
or R₆ is represented by the structure of formula Bi: 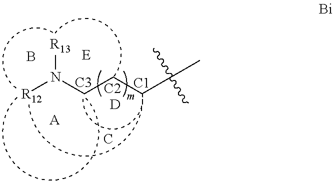
wherein
m is 0 or 1; and
R₁₂ is R₂₀ or C₁-C₅ C(O)-alkyl, and R₁₃ is R₃₀; or
R₁₂ and R₁₃ are both H; or
R₁₂ and R₁₃ are each independently H or substituted or unsubstituted C₁-C₅ alkyl; or
R₁₂ and C3 are joined to form ring A and R₁₃ is R₃₀; or
R₁₂ and R₁₃ are joined to form ring B; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
Ring A, C and E are each independently a substituted or unsubstituted single, spiro or fused 3-8 membered heterocyclic ring;
Ring B is a substituted or unsubstituted single, spiro or fused 3-8 membered heterocyclic ring; and
Ring D is a substituted or unsubstituted C₃-C₈ cycloalkyl;
or R₆ and R₅ are joined to for a substituted or unsubstituted 5-8 membered heterocyclic ring;
R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl, C₁-C₅ linear or branched alkoxy, C₁-C₅ linear or branched haloalkyl, R₈-aryl, —R₈—O—R₈—O—R₁₀, —R₈—O—R₁₀, —R₈-R₁₀, substituted or unsubstituted aryl, substituted or unsubstituted heteroaryl;
AND
R₇ is H, F, Cl, Br, I, OH, O—R₂₀, SH, R₈—OH, R₈—SH, SR₁₀, —R₈—O—R₁₀, —R₈—S—R₁₀, R₈—(C₃-C₈ cycloalkyl), R₈-(substituted or unsubstituted single, fused or spiro 3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, cyclopropanol, cyclohexyl, substituted or unsubstituted 4-7 membered heterocyclic ring, tetrahydrofuran, oxetane, oxetan-3-ol, pyrrolidine, 1-methylpyrrolidine, pyrrolidin-2-one, piperidine, piperidine-4-carbonitrile, 4-fluoropiperidine, substituted or unsubstituted aryl, substituted or unsubstituted benzyl;
or R₇ is represented by the structure of formula A: 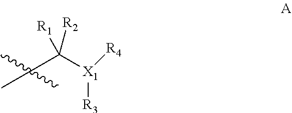
wherein
X₁ is N or O;
R₁ and R₂ are each independently H, F, or CF₃; or R₁ and R₂ are joined to form a C₃-C₈ carbocyclic or heterocyclic ring;
R₃ and R₄ are each independently H, Me, substituted or unsubstituted C₁-C₅ alkyl, substituted or unsubstituted C₃-C₈ cycloalkyl, cyclopropyl, substituted or unsubstituted 5-7 membered heterocyclic ring, or R₂₀; or R₃ and R₄ are joined to form a 3-8 membered heterocyclic ring;
wherein if X₁ is O then R₄ is absent;
AND
R₇′ is H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, methyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, cyclopropyl, substituted or unsubstituted 3-8 membered heterocyclic ring, substituted or unsubstituted aryl, substituted or unsubstituted benzyl;
or R₇ and R₇′ are joined to form a 5 or 6 membered substituted or unsubstituted, saturated, unsaturated or aromatic, carbocyclic or heterocyclic ring, piperidine, pyrrolidine, tetrahydrofuran, or tetrahydropyran;
wherein at least one of X₂, X₃, X₄, X₅, X₆, X₇, X₈, X₉ or X₁₀ is N;
OR
5.
R₅ and R₆ are joined to for a substituted or unsubstituted 5-7 membered heterocyclic ring (e.g., azepam, piperazine);
AND
R₇ is H, F, Cl, Br, I, OH, O—R₂₀, SH, R₈—OH, R₈—SH, SR₁₀, —R₈—O—R₁₀, —R₈—S—R₁₀, R₈—(C₃-C₈ cycloalkyl), R₈-(substituted or unsubstituted single, fused or spiro 3-8 membered heterocyclic ring), CF₃, CD₃, OCD₃, CN, —CH₂CN, —R₈CN, R₈—N(R₁₀)(R₁₁), R₉—R₈—N(R₁₀)(R₁₁), B(OH)₂, —OCH₂Ph, COOH, C(O)H, —C(O)NH₂, SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, cyclopropanol, cyclohexyl, substituted or unsubstituted 4-7 membered heterocyclic ring, tetrahydrofuran, oxetane, oxetan-3-ol, pyrrolidine, 1-methylpyrrolidine, pyrrolidin-2-one, piperidine, piperidine-4-carbonitrile, 4-fluoropiperidine, substituted or unsubstituted aryl, substituted or unsubstituted benzyl;
or R₇ is represented by the structure of formula A: 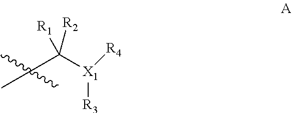
wherein
X₁ is N or O;
R₁ and R₂ are each independently H, F, or CF₃; or R₁ and R₂ are joined to form a C₃-C₈ carbocyclic or heterocyclic ring;
R₃ and R₄ are each independently H, Me, substituted or unsubstituted C₁-C₅ alkyl, substituted or unsubstituted C₃-C₈ cycloalkyl, substituted or unsubstituted 5-7 membered heterocyclic ring, or R₂₀; or R₃ and R₄ are joined to form a 3-8 membered heterocyclic ring;
wherein if X₁ is O then R₄ is absent;
AND
R₇′ is H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, methyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, cyclopropyl, substituted or unsubstituted 3-8 membered heterocyclic ring, substituted or unsubstituted aryl, substituted or unsubstituted benzyl;
or R₇ and R₇′ are joined to form a 5 or 6 membered substituted or unsubstituted, saturated, unsaturated or aromatic, carbocyclic or heterocyclic ring, piperidine, pyrrolidine, tetrahydrofuran, or tetrahydropyran;
or its pharmaceutically acceptable salt, optical isomer, tautomer, hydrate, N-oxide, reverse amide analog, isotopic variant such as: deuterated analog), pharmaceutical product or any combination thereof.

5. The compound of claim 4, selected from the following:

| Compound | |
|---|---|
| No. | Compound Name |
| 102 | Azepan-1-yl(2-(p-tolyl)benzo[d]imidazo[2,1-b]thiazol-7-yl)methanone |
| 104 | azepan-1-yl(2-(4-fluorophenyl)benzo[d]imidazo[2,1-b]thiazol-7-yl)methanone |
| 105 | 2-(4-fluorophenyl)-N-(3-(propylthio)propyl)benzo[d]imidazo[2,1-b]thiazole-7- |
| | carboxamide |
| 106 | azepan-1-yl(2-(4-ethoxyphenyl)benzo[d]imidazo[2,1-b]thiazol-7-yl)methanone |
| 110 | N-(3-acetamidopropyl)-2-(p-tolyl)benzo[d]imidazo[2,1-b]thiazole-7-carboxamide |
| 114 | (S)-N-(pyrrolidin-3-ylmethyl)-2-(p-tolyl)benzo[d]imidazo[2,1-b]thiazole-7- |
| | carboxamide formate |
| 115 | N-(3-aminopropyl)-2-(p-tolyl)benzo[d]imidazo[2,1-b]thiazole-7-carboxamide |
| 116 | (R)-N-(pyrrolidin-3-ylmethyl)-2-(p-tolyl)benzo[d]imidazo[2,1-b]thiazole-7- |
| | carboxamide formate |
| 117 | N-(azetidin-3-ylmethyl)-2-(p-tolyl)benzo[d]imidazo[2,1-b]thiazole-7-carboxamide |
| 119 | (S)-N-((1-ethylpyrrolidin-2-yl)methyl)-2-(3-methoxyphenyl)benzo[d]imidazo[2,1- |
| | b]thiazole-7-carboxamide |
| 122 | N-(2-aminoethyl)-2-(p-tolyl)benzo[d]imidazo[2,1-b]thiazole-7-carboxamide |
| 127 | (R)-N-((1-ethylpyrrolidin-2-yl)methyl)-2-(3-methoxyphenyl)benzo[d]imidazo[2,1- |
| | b]thiazole-7-carboxamide |
| 135 | N-(3-(1,1-dioxidothiomorpholino)propyl)-2-(m-tolyl)benzo[d]imidazo[2,1- |
| | b]thiazole-7-carboxamide |
| 138 | N-((1s,3s)-3-(piperidin-1-yl)cyclobutyl)-2-(m-tolyl)benzo[d]imidazo[2,1-b]thiazole- |
| | 7-carboxamide |
| 139 | N-(3-(tetrahydro-2H-pyran-4-yl)propyl)-2-(m-tolyl)benzo[d]imidazo[2,1-b]thiazole- |
| | 7-carboxamide |
| 140 | N-(piperidin-4-yl)-2-(m-tolyl)benzo[d]imidazo[2,1-b]thiazole-7-carboxamide |
| 141 | piperazin-1-yl(2-(m-tolyl)benzo[d]imidazo[2,1-b]thiazol-7-yl)methanone |
| 149 | N-(3-(pyrrolidin-1-yl)propyl)-2-(m-tolyl)benzo[d]imidazo[2,1-b]thiazole-7- |
| | carboxamide |
| 151 | N-((1r,3r)-3-(piperidin-1-yl)cyclobutyl)-2-(m-tolyl)benzo[d]imidazo[2,1-b]thiazole- |
| | 7-carboxamide |
| 153 | 2-([1,1′-biphenyl]-3-yl)-N-(3-(diethylamino)propyl)benzo[d]imidazo[2,1-b]thiazole- |
| | 7-carboxamide |
| 156 | N-(2-(pyrrolidin-2-yl)ethyl)-2-(m-tolyl)benzo[d]imidazo[2,1-b]thiazole-7- |
| | carboxamide |
| 157 | N-((1s,3s)-3-(methylamino)cyclobutyl)-2-(m-tolyl)benzo[d]imidazo[2,1-b]thiazole-7- |
| | carboxamide formate |
| 158 | N-((1r,3r)-3-(methylamino)cyclobutyl)-2-(m-tolyl)benzo[d]imidazo[2,1-b]thiazole-7- |
| | carboxamide formate |
| 159 | N-(3-oxo-3-(pyrrolidin-1-yl)propyl)-2-(m-tolyl)benzo[d]imidazo[2,1-b]thiazole-7- |
| | carboxamide |
| 161 | N-(3-(diethylamino)propyl)-2-(pyridin-4-yl)benzo[d]imidazo[2,1-b]thiazole-7- |
| | carboxamide |
| 165 | N-(3-(diethylamino)propyl)-2-(5-methylpyridin-3-yl)benzo[d]imidazo[2,1- |
| | b]thiazole-7-carboxamide |
| 168 | N-(2-(m-tolyl)benzo[d]imidazo[2,1-b]thiazol-7-yl)piperidine-4-carboxamide |
| 170 | N-(3-(diethylamino)propyl)-2-(3-morpholinophenyl)benzo[d]imidazo[2,1-b]thiazole- |
| | 7-carboxamide |
| 171 | N-(3-(diethylamino)propyl)-2-(3-(pyrrolidin-1-yl)phenyl)benzo[d]imidazo[2,1- |
| | b]thiazole-7-carboxamide |
| 173 | N-(3-(diethylamino)propyl)-2-(4-(oxetan-3-yl)phenyl)benzo[d]imidazo[2,1- |
| | b]thiazole-7-carboxamide |
| 179 | N-(3-(diethylamino)propyl)-2-(4-(2-oxopyrrolidin-1-yl)phenyl)benzo[d]imidazo[2,1- |
| | b]thiazole-7-carboxamide |
| 248 | N-(3-(5-methyl-2,5-diazabicyclo[2.2.1]heptan-2-yl)propyl)-2-(m- |
| | tolyl)benzo[d]imidazo[2,1-b]thiazole-7-carboxamide |
| 265 | N-(2-(m-tolyl)benzo[d]imidazo[2,1-b]thiazol-7-yl)piperazine-1-carboxamide |
| 266 | 4-(diethylamino)-N-(2-(m-tolyl)benzo[d]imidazo[2,1-b]thiazol-7-yl)butanamide |
| 267 | 1-(2-(diethylamino)ethyl)-3-(2-(m-tolyl)benzo[d]imidazo[2,1-b]thiazol-7-yl)urea |
| 277 | N-(3-(diethylamino)propyl)-2-(4-(hydroxymethyl)phenyl)benzo[d]imidazo[2,1- |
| | b]thiazole-7-carboxamide |
| 299 | 2-(3-(aminomethyl)phenyl)-N-(3-(diethylamino)propyl)benzo[d]imidazo[2,1- |
| | b]thiazole-7-carboxamide |
| 304 | N-(3-(diethylamino)propyl)-2-(4- |
| | (methylcarbamoyl)phenyl)imidazo[2′,1′:2,3]thiazolo[5,4-b]pyridine-7-carboxamide |
| 305 | 2-(4-(methylcarbamoyl)phenyl)-N-(3-(piperidin-1- |
| | yl)propyl)imidazo[2′,1′:2,3]thiazolo[5,4-b]pyridine-7-carboxamide |
| 306 | N-(3-(piperidin-1-yl)propyl)-2-(pyridin-4-yl)benzo[d]imidazo[2,1-b]thiazole-7- |
| | carboxamide |
| 307 | N-(3-(diethylamino)propyl)-2-(4-morpholinophenyl)benzo[d]imidazo[2,1-b]thiazole- |
| | 7-carboxamide |
| 308 | N-(3-(diethylamino)propyl)-2-(3-(oxetan-3-yl)phenyl)benzo[d]imidazo[2,1- |
| | b]thiazole-7-carboxamide |
| 313 | N-(3-(diethylamino)propyl)-2-(pyridazin-4-yl)benzo[d]imidazo[2,1-b]thiazole-7- |
| | carboxamide |
| 314 | N-(3-(diethylamino)propyl)-2-(4-(tetrahydro-2H-pyran-4- |
| | yl)phenyl)benzo[d]imidazo[2,1-b]thiazole-7-carboxamide |
| 315 | 2-(4-(oxetan-3-yl)phenyl)-N-(3-(piperidin-1-yl)propyl)benzo[d]imidazo[2,1- |
| | b]thiazole-7-carboxamide |
| 316 | N-(3-(diethylamino)propyl)-2-(4- |
| | (methylcarbamoyl)phenyl)imidazo[2′,1′:2,3]thiazolo[4,5-b]pyridine-7-carboxamide |
| 317 | 2-(4-(methylcarbamoyl)phenyl)-N-(3-(piperidin-1- |
| | yl)propyl)imidazo[2′,1′:2,3]thiazolo[4,5-b]pyridine-7-carboxamide |
| 319 | 2-(4-(methylcarbamoyl)phenyl)-N-(3-(piperidin-1-yl)propyl)benzo[4,5]thiazolo[3,2- |
| | b][1,2,4]triazole-6-carboxamide |
| 324 | (R)-N-((1-(2-(3-(but-3-yn-1-yl)-3H-diazirin-3-yl)ethyl)pyrrolidin-3-yl)methyl)-2-(p- |
| | tolyl)benzo[d]imidazo[2,1-b]thiazole-7-carboxamide |
| 325 | (S)-N-((1-(2-(3-(but-3-yn-1-yl)-3H-diazirin-3-yl)ethyl)pyrrolidin-3-yl)methyl)-2-(p- |
| | tolyl)benzo[d]imidazo[2,1-b]thiazole-7-carboxamide |
| 326 | 2-(2-(2-(3-(but-3-yn-1-yl)-3H-diazirin-3-yl)ethoxy)phenyl)-N-(3-(piperidin-1- |
| | yl)propyl)benzo[d]imidazo[2,1-b]thiazole-7-carboxamide |
| 327 | 2-(3-(2-(3-(but-3-yn-1-yl)-3H-diazirin-3-yl)ethoxy)phenyl)-N-(3- |
| | (diethylamino)propyl)benzo[d]imidazo[2,1-b]thiazole-7-carboxamide |
| 328 | N-(3-(diethylamino)propyl)-2-(4-(methylcarbamoyl)phenyl)benzo[4,5]thiazolo[3,2- |
| | b][1,2,4]triazole-6-carboxamide |
| 347 | 2-(4-((1H-imidazol-2-yl)methyl)phenyl)-N-(3- |
| | (diethylamino)propyl)benzo[d]imidazo[2,1-b]thiazole-7-carboxamide |
| 349 | N-(3-(diethylamino)propyl)-2-(2-fluoro-4- |
| | (methylcarbamoyl)phenyl)benzo[4,5]thiazolo[3,2-b][1,2,4]triazole-6-carboxamide |
| 355 | N-(3-(diethylamino)propyl)-2-(2-fluoro-4- |
| | (methylcarbamoyl)phenyl)imidazo[2′,1′:2,3]thiazolo[4,5-b]pyridine-7-carboxamide |
| 357 | N-(3-(diethylamino)propyl)-2-(2-fluoro-4- |
| | (methylcarbamoyl)phenyl)imidazo[2′,1′:2,3]thiazolo[5,4-b]pyridine-7-carboxamide |
| 358 | N-(3-(diethylamino)propyl)-7-(4- |
| | (methylcarbamoyl)phenyl)imidazo[2′,1′:2,3]thiazolo[5,4-d]pyrimidine-2- |
| | carboxamide |
| 360 | N-(3-(diethylamino)propyl)-7-(2-fluoro-4- |
| | (methylcarbamoyl)phenyl)imidazo[2′,1′:2,3]thiazolo[5,4-d]pyrimidine-2- |
| | carboxamide |
| 361 | 7-(4-(methylcarbamoyl)phenyl)-N-(3-(piperidin-1- |
| | yl)propyl)imidazo[2′,1′:2,3]thiazolo[5,4-d]pyrimidine-2-carboxamide |
| 366 | N-(3-(diethylamino)propyl)-2-(3-fluoropyridin-4-yl)benzo[d]imidazo[2,1-b[thiazole- |
| | 7-carboxamide |
| 367 | N-(3-(diethylamino)propyl)-2-(2-fluoro-4-(oxetan-3-yl)phenyl)benzo[d]imidazo[2,1- |
| | b]thiazole-7-carboxamide |
| 368 | 2-(4-((cyclopropylamino)methyl)-2-fluorophenyl)-N-(3-(piperidin-1- |
| | yl)propyl)benzo[d]imidazo[2,1-b]thiazole-7-carboxamide |
| 374 | 2-(4-(aminomethyl)-2-fluorophenyl)-N-(3-(piperidin-1- |
| | yl)propyl)benzo[d]imidazo[2,1-b]thiazole-7-carboxamide |
| 376 | 2-(4-(aminomethyl)-3-chlorophenyl)-N-(3-(piperidin-1- |
| | yl)propyl)benzo[d]imidazo[2,1-b]thiazole-7-carboxamide |
| 379 | 2-(4-(aminomethyl)-3-(difluoromethyl)phenyl)-N-(3-(piperidin-1- |
| | yl)propyl)benzo[d]imidazo[2,1-b]thiazole-7-carboxamide |
| 386 | 2-(2-fluoro-4-(methylcarbamoyl)phenyl)-N-(3-(piperidin-1- |
| | yl)propyl)benzo[4,5]thiazolo[3,2-b][1,2,4]triazole-6-carboxamide |
| 390 | 2-(4-(aminomethyl)-2-fluorophenyl)-N-(3-(4-fluoropiperidin-1- |
| | yl)propyl)benzo[4,5]thiazolo[3,2-b][1,2,4]triazole-6-carboxamide |
| 391 | 2-(2-fluoro-4-(methylcarbamoyl)phenyl)-N-(3-(pyrrolidin-1- |
| | yl)propyl)benzo[4,5]thiazolo[3,2-b][1,2,4]triazole-6-carboxamide |
| 400 | N-(3-(diethylamino)propyl)-2-(4- |
| | (methylcarbamoyl)phenyl)imidazo[2′,1′:2,3]thiazolo[4,5-c]pyridine-7-carboxamide |
| 403 | N-(3-(diethylamino)propyl)-2-(2-fluoro-4- |
| | (methylcarbamoyl)phenyl)imidazo[2′,1′:2,3]thiazolo[4,5-c]pyridine-7-carboxamide |
| 406 | 2-(4-(methylcarbamoyl)phenyl)-N-(3-(piperidin-1- |
| | yl)propyl)imidazo[2′,1′:2,3]thiazolo[4,5-c]pyridine-7-carboxamide |
| 409 | 2-(2-fluoro-4-(methylcarbamoyl)phenyl)-N-(3-(piperidin-1- |
| | yl)propyl)imidazo[2′,1′:2,3]thiazolo[4,5-c]pyridine-7-carboxamide |
| 412 | (R)-N-(3-(piperidin-1-yl)propyl)-2-(4-(pyrrolidin-2-yl)phenyl)benzo[d]imidazo[2,1- |
| | b]thiazole-7-carboxamide |
| 413 | (S)-N-(3-(piperidin-1-yl)propyl)-2-(4-(pyrrolidin-2-yl)phenyl)benzo[d]imidazo[2,1- |
| | b]thiazole-7-carboxamide |
| 414 | (R)-N-(3-(diethylamino)propyl)-2-(4-(pyrrolidin-2-yl)phenyl)benzo[d]imidazo[2,1- |
| | b]thiazole-7-carboxamide |
| 415 | (S)-N-(3-(diethylamino)propyl)-2-(4-(pyrrolidin-2-yl)phenyl)benzo[d]imidazo[2,1- |
| | b]thiazole-7-carboxamide |
| 416 | 2-(2-fluoro-4-(pyrrolidin-2-yl)phenyl)-N-(3-(piperidin-1- |
| | yl)propyl)benzo[d]imidazo[2,1-b]thiazole-7-carboxamide |
| 417 | (R)-2-(2-fluoro-4-(pyrrolidin-2-yl)phenyl)-N-(3-(piperidin-1- |
| | yl)propyl)benzo[d]imidazo[2,1-b]thiazole-7-carboxamide |
| 418 | (S)-2-(2-fluoro-4-(pyrrolidin-2-yl)phenyl)-N-(3-(piperidin-1- |
| | yl)propyl)benzo[d]imidazo[2,1-b]thiazole-7-carboxamide |
| 419 | (R)-2-(2-fluoro-4-(pyrrolidin-2-yl)phenyl)-N-(3-(4-fluoropiperidin-1- |
| | yl)propyl)benzo[d]imidazo[2,1-b]thiazole-7-carboxamide |
| 420 | (S)-2-(2-fluoro-4-(pyrrolidin-2-yl)phenyl)-N-(3-(4-fluoropiperidin-1- |
| | yl)propyl)benzo[d]imidazo[2,1-b]thiazole-7-carboxamide |
| 422 | (S)-2-(2-fluoro-4-(pyrrolidin-2-yl)phenyl)-N-(3-(piperidin-1- |
| | yl)propyl)benzo[4,5]thiazolo[3,2-b][1,2,4]triazole-6-carboxamide |
| 425 | 2-(2-fluoro-4-(3-hydroxyoxetan-3-yl)phenyl)-N-(3-(4-fluoropiperidin-1- |
| | yl)propyl)benzo[d]imidazo[2,1-b]thiazole-7-carboxamide |
| 428S | (S)-2-(2-fluoro-4-(tetrahydrofuran-2-yl)phenyl)-N-(3-(4-fluoropiperidin-1- |
| | yl)propyl)benzo[d]imidazo[2,1-b]thiazole-7-carboxamide |
| 428R | (R)-2-(2-fluoro-4-(tetrahydrofuran-2-yl)phenyl)-N-(3-(4-fluoropiperidin-1- |
| | yl)propyl)benzo[d]imidazo[2,1-b]thiazole-7-carboxamide |
| 434S | (S)-2-(2-fluoro-4-(pyrrolidin-3-yl)phenyl)-N-(3-(4-fluoropiperidin-1- |
| | yl)propyl)benzo[d]imidazo[2,1-b]thiazole-7-carboxamide |
| 434R | (R)-2-(2-fluoro-4-(pyrrolidin-3-yl)phenyl)-N-(3-(4-fluoropiperidin-1- |
| | yl)propyl)benzo[d]imidazo[2,1-b]thiazole-7-carboxamide |
| 435S | (S)-2-(2-fluoro-4-(tetrahydrofuran-3-yl)phenyl)-N-(3-(4-fluoropiperidin-1- |
| | yl)propyl)benzo[d]imidazo[2,1-b]thiazole-7-carboxamide |
| 435R | (R)-2-(2-fluoro-4-(tetrahydrofuran-3-yl)phenyl)-N-(3-(4-fluoropiperidin-1- |
| | yl)propyl)benzo[d]imidazo[2,1-b]thiazole-7-carboxamide |
| 437S | (S)-2-(2-fluoro-5-(pyrrolidin-2-yl)phenyl)-N-(3-(4-fluoropiperidin-1- |
| | yl)propyl)benzo[d]imidazo[2,1-b]thiazole-7-carboxamide |
| 437R | (R)-2-(2-fluoro-5-(pyrrolidin-2-yl)phenyl)-N-(3-(4-fluoropiperidin-1- |
| | yl)propyl)benzo[d]imidazo[2,1-b]thiazole-7-carboxamide |
| 438S | (S)-2-(2-fluoro-6-(pyrrolidin-2-yl)phenyl)-N-(3-(4-fluoropiperidin-1- |
| | yl)propyl)benzo[d]imidazo[2,1-b]thiazole-7-carboxamide |
| 438R | (R)-2-(2-fluoro-6-(pyrrolidin-2-yl)phenyl)-N-(3-(4-fluoropiperidin-1- |
| | yl)propyl)benzo[d]imidazo[2,1-b]thiazole-7-carboxamide |
| 439S | (S)-2-(2-fluoro-3-(pyrrolidin-2-yl)phenyl)-N-(3-(4-fluoropiperidin-1- |
| | yl)propyl)benzo[d]imidazo[2,1-b]thiazole-7-carboxamide |
| 439R | (R)-2-(2-fluoro-3-(pyrrolidin-2-yl)phenyl)-N-(3-(4-fluoropiperidin-1- |
| | yl)propyl)benzo[d]imidazo[2,1-b]thiazole-7-carboxamide |
| 440 | 2-(2-fluoro-4-(1-hydroxycyclopropyl)phenyl)-N-(3-(4-fluoropiperidin-1- |
| | yl)propyl)benzo[d]imidazo[2,1-b]thiazole-7-carboxamide |
| 441 | 2-(2-fluoro-4-(3-hydroxyoxetan-3-yl)phenyl)-N-(3-(4-fluoropiperidin-1- |
| | yl)propyl)benzo[d]imidazo[2,1-b]thiazole-7-carboxamide |
| 446 | N-(3-(4-fluoro-2-oxopiperidin-1-yl)propyl)-2-(2-fluoro-4-((S)-tetrahydrofuran-2- |
| | yl)phenyl)benzo[d]imidazo[2,1-b]thiazole-7-carboxamide |
| 447 | N-(3-(4-fluoro-2-oxopiperidin-1-yl)propyl)-2-(2-fluoro-4-((S)-tetrahydrofuran-2- |
| | yl)phenyl)benzo[d]imidazo[2,1-b]thiazole-7-carboxamide |
| 465 | (S)-3-cyclopropyl-N-(3-(4-fluoropiperidin-1-yl)propyl)-2-(4-(tetrahydrofuran-2- |
| | yl)phenyl)benzo[d]imidazo[2,1-b]thiazole-7-carboxamide |
| 466 | (R)-3-cyclopropyl-N-(3-(4-fluoropiperidin-1-yl)propyl)-2-(4-(tetrahydrofuran-2- |
| | yl)phenyl)benzo[d]imidazo[2,1-b]thiazole-7-carboxamide |

6. The compound according to claim 4, wherein R₆ is represented by the structure of formula Bi; R₇ is represented by the structure of formula A; or combination thereof.

7. The compound of claim 6, wherein
R₁₂ and R₁₃ are joined to form a pyrrolidine ring or a substituted or unsubstituted piperidine ring;
C3 and R₁₂ are joined to form a pyrrolidine (ring A); or
C2 and R₁₃ are joined to form a piperidine (ring E).

8. The compound of claim 7, wherein R₁ is H; R₂ is H or CF₃, or R₁ and R₂ are joined to form a cyclopropyl; X₁ is N; R₃ is H; and R₄ is H or cycloalkyl.

9. The compound of claim 4, wherein R₇ is CH₂—NH₂; R₇′ is H or F; R₅ is H; R₆ is R₈—N(R₁₀)(R₁₁), preferably wherein R₈ is (CH₂CH₂CH₂); R₁₀ and R₁₁ are joined to form a substituted or unsubstituted 3-8 membered heterocyclic ring, preferably piperidine, or are C₁-C₅ unsubstituted linear alkyl, preferably ethyl; or any combination thereof.

10. A compound represented by the structure of formula I(f): 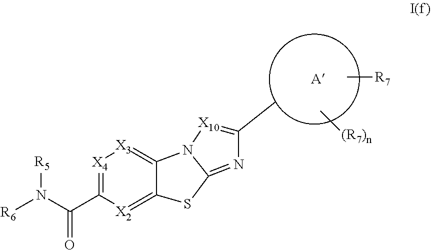
wherein
A′ is a 3-8 membered single or fused saturated, unsaturated or aromatic heterocyclic ring;
X₂, X₃, and X₄, are each independently nitrogen or CH;
X₁₀ is N, CH, or C(R);
R₅ is H or C₁-C₅ linear or branched alkyl;
R₆ is H, F, Cl, Br, I, OH, SH, R₈—OH, R₈—SH, —R₈—O—R₁₀, R₈—S—R₁₀, (CH₂)₃—S—(CH₂)₂CH₃), R₈—NHC(O)—R₁₀, —O—R₈—R₁₀, R₈-(substituted or unsubstituted C₃-C₈ cycloalkyl), R₈-(substituted or unsubstituted saturated, unsaturated or aromatic, single, fused or spiro 3-10 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), (CH₂)₃—N(CH₂CH₃)₂, (CH₂)₃—NH₂, (CH₂)₃—N(CH₂CH₃)(CH₂CF₃, R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, R₈-(substituted or unsubstituted C₃-C₈ cycloalkyl), substituted or unsubstituted 3-8 membered heterocyclic ring, substituted or unsubstituted aryl, substituted or unsubstituted R₈-aryl, or substituted or unsubstituted benzyl;
or R₆ is represented by the structure of formula B: 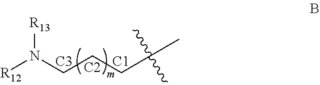
wherein
m is 0 or 1; and
R₁₂ is R₂₀ or C₁-C₅ C(O)-alkyl, and R₁₃ is R₃₀; or
R₁₂ and R₁₃ are both H; or
R₁₂ and R₁₃ are each independently H or substituted or unsubstituted C₁-C₅ alkyl; or
R₁₂ and C3 are joined to form ring A and R₁₃ is R₃₀; or
R₁₂ and R₁₃ are joined to form ring B; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
Ring A, C and E are each independently a substituted or unsubstituted single, spiro or fused 3-8 membered heterocyclic ring;
Ring B is a substituted or unsubstituted single, spiro or fused 3-8 membered heterocyclic ring; and
Ring D is a substituted or unsubstituted C₃-C₈ cycloalkyl;
or R₆ and R₅ are joined to for a substituted or unsubstituted 5-8 membered heterocyclic ring;
R₇ is H, F, Cl, Br, I, OH, O—R₂₀, SH, R₈—OH, R₈—SH, SR₁₀, —R₈—O—R₁₀, —R₈—S—R₁₀, R₈—(C₃-C₈ cycloalkyl), R₈-(substituted or unsubstituted single, fused or spiro 3-8 membered heterocyclic ring), CF₃, CD₃, OCD₃, CN, —CH₂CN, —R₈CN, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O-R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkyl, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted 4-7 membered heterocyclic ring, substituted or unsubstituted aryl, substituted or unsubstituted benzyl;
or R₇ is represented by the structure of formula A: 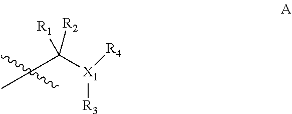
wherein
X₁ is N or O;
R₁ and R₂ are each independently H, F, or CF₃; or R₁ and R₂ are joined to form ═O, or a C₃-C₈ carbocyclic or heterocyclic ring;
R₃ and R₄ are each independently H, Me, substituted or unsubstituted C₁-C₅ alkyl, substituted or unsubstituted C₃-C₈ cycloalkyl, substituted or unsubstituted 5-7 membered heterocyclic ring, or R₂₀; or R₃ and R₄ are joined to form a 3-8 membered heterocyclic ring;
wherein if X₁ is O then R₄ is absent;
R₇′ is H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, —CH₂CN, —R₈CN, R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted 3-8 membered heterocyclic ring, substituted or unsubstituted aryl, substituted or unsubstituted benzyl;
or R₇ and R₇′ are joined to form a 5 or 6 membered substituted or unsubstituted, saturated, unsaturated or aromatic, carbocyclic or heterocyclic ring;
R₂₀ is represented by the following structure: 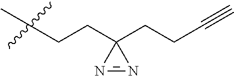 [SMILES: *CCC1(CCC#C)N=N1]
R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl, C₁-C₅ linear or branched alkoxy, C₁-C₅ linear or branched haloalkyl, R₈-aryl, —R₈—O—R₈—O—R₁₀, —R₈—O—R₁₀, —R₈-R₁₀, substituted or unsubstituted aryl, substituted or unsubstituted heteroaryl;
R is H, F, Cl, Br, I, OH, SH, OH, alkoxy, NH(R₁₀), NH—CH₂-cyclopropyl, N(R₁₀)(R₁₁), CF₃, CN, NO₂, COOH, C₁-C₅ linear or branched, substituted or unsubstituted alkyl, methyl, ethyl, CH₂—OH, CH₂—CH₂—OH, C₃-C₈ substituted or unsubstituted cycloalkyl, cyclopropyl, C₁-C₅ linear or branched alkoxy, isopropoxy, C₁-C₅ linear or branched haloalkyl, R₈-aryl, —R₈—O—R₈—O—R₁₀, —R₈—O—R₁₀, —R₈-R₁₀, substituted or unsubstituted aryl, substituted or unsubstituted heteroaryl;
each R₈ is independently [CH₂]ₚ
wherein p is between 1 and 10;
R₉ is [CH]_{q}, [C]_{q}
wherein q is between 2 and 10;
R₁₀ and R₁₁ are each independently H, C₁-C₅ substituted or unsubstituted linear or branched alkyl, methyl, CH₂CF₃, C₁-C₅ linear or branched alkoxy, C(O)R, or S(O)₂R;
or R₁₀ and R₁₁ are joined to form a substituted or unsubstituted 3-8 membered heterocyclic ring such as: piperazine, piperidine),
n is an integer between 0 and 4 (e.g., 1, 2);
or its pharmaceutically acceptable salt, stereoisomer, tautomer, hydrate, N-oxide, reverse amide analog, isotopic variant (e.g., deuterated analog), pharmaceutical product or any combination thereof.

11. The compound of claim 10, represented by the following structure:

| Compound | |
|---|---|
| No. | Compound name |
| 162 | N-(3-(diethylamino)propyl)-2-morpholinobenzo[d]imidazo[2,1-b]thiazole-7- |
| | carboxamide |
| 335 | N-(3-(diethylamino)propyl)-2-(piperazin-1-yl)benzo[d]imidazo[2,1-b]thiazole-7- |
| | carboxamide |
| 336 | N-(3-(diethylamino)propyl)-2-(piperidin-1-yl)benzo[d]imidazo[2,1-b]thiazole-7- |
| | carboxamide |
| 337 | N-(3-(diethylamino)propyl)-2-(4-methylpiperazin-1-yl)benzo[d]imidazo[2,1- |
| | b]thiazole-7-carboxamide |
| 454 | 2-(6-fluoroisoindolin-5-yl)-N-(3-(4-fluoropiperidin-1- |
| | yl)propyl)benzo[d]imidazo[2,1-b]thiazole-7-carboxamide |
| 455 | 2-(7-fluoro-1,2,3,4-tetrahydroisoquinolin-6-yl)-N-(3-(4-fluoropiperidin-1- |
| | yl)propyl)benzo[d]imidazo[2,1-b]thiazole-7-carboxamide |
| 456 | 2-(6-fluoro-1,3-dihydroisobenzofuran-5-yl)-N-(3-(4-fluoropiperidin-1- |
| | yl)propyl)benzo[d]imidazo[2,1-b]thiazole-7-carboxamide |
| 457 | 2-(7-fluoroisochroman-6-yl)-N-(3-(4-fluoropiperidin-1- |
| | yl)propyl)benzo[d]imidazo[2,1-b]thiazole-7-carboxamide |
| 458 | 2-(7-fluoroindolin-6-yl)-N-(3-(4-fluoropiperidin-1-yl)propyl)benzo[d]imidazo[2,1- |
| | b]thiazole-7-carboxamide |
| 459 | 2-(4-fluoroindolin-5-yl)-N-(3-(4-fluoropiperidin-1-yl)propyl)benzo[d]imidazo[2,1- |
| | b]thiazole-7-carboxamide |
| 460S | (S)-2-(2-fluoro-4-(pyrrolidin-2-yl)-5,6,7,8-tetrahydronaphthalen-1-yl)-N-(3-(4- |
| | fluoropiperidin-1-yl)propyl)benzo[d]imidazo[2,1-b]thiazole-7-carboxamide |
| 460R | (R)-2-(2-fluoro-4-(pyrrolidin-2-yl)-5,6,7,8-tetrahydronaphthalen-1-yl)-N-(3-(4- |
| | fluoropiperidin-1-yl)propyl)benzo[d]imidazo[2,1-b]thiazole-7-carboxamide |
| 461S | (S)-2-(5-fluoro-7-(pyrrolidin-2-yl)-2,3-dihydro-1H-inden-4-yl)-N-(3-(4- |
| | fluoropiperidin-1-yl)propyl)benzo[d]imidazo[2,1-b]thiazole-7-carboxamide |
| 461R | (R)-2-(5-fluoro-7-(pyrrolidin-2-yl)-2,3-dihydro-1H-inden-4-yl)-N-(3-(4- |
| | fluoropiperidin-1-yl)propyl)benzo[d]imidazo[2,1-b]thiazole-7-carboxamide |

12. A compound represented by the structure of formula I(g): 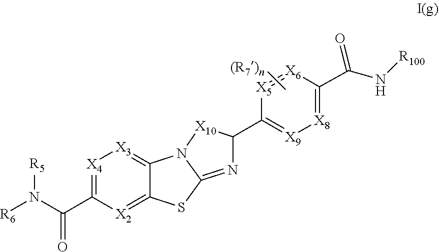
wherein
X₂, X₃, and X₄, are each independently nitrogen or CH;
X₅, X₆, X₇, X₈ and X₉ are each independently nitrogen or carbon atoms;
X₁₀ is N, CH, or C(R);
R₁₀₀ is a C₁-C₅ linear or branched, substituted or unsubstituted alkyl, R₈—OH, —R₈—O—R₁₀, R₈—N(R₁₀)(R₁₁), R₂₀, or a substituted or unsubstituted 3-8 membered heterocyclic ring;
R₅ is H or C₁-C₅ linear or branched alkyl;
R₆ is F, Cl, Br, I, OH, SH, R₈—OH, R₈—SH, —R₈—O—R₁₀, R₈—S—R₁₀, R₈—NHC(O)—R₁₀, —O—R₈—R₁₀, R₈-(substituted or unsubstituted C₃-C₈ cycloalkyl), R₈-(substituted or unsubstituted 3 to 8 membered single, fused or spiro heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₈—C(O)N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)-R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, substituted or unsubstituted C₁-C₅ linear or branched, or C₃-C₈ cyclic alkoxy optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, R₈-(substituted or unsubstituted C₃-C₈ cycloalkyl), substituted or unsubstituted 3-8 membered heterocyclic ring, substituted or unsubstituted aryl, substituted or unsubstituted R₈-aryl, substituted or unsubstituted benzyl;
or R₆ and R₅ are joined to for a substituted or unsubstituted 5-8 membered heterocyclic ring;
or R₆ is represented by the structure of formula C: 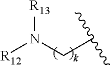
wherein
k is between 1 and 4;
R₁₂ and R₁₃ are each independently H, C₁-C₅ linear or branched, substituted or unsubstituted alkyl, R₂₀, or
R₁₂ and R₁₃ are joined to form a substituted or unsubstituted 4-7 membered heterocyclic ring;
or R₆ is represented by the structure of formula Bi: 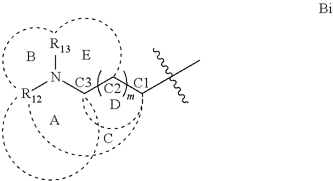
wherein
m is 0 or 1; and
R₁₂ is R₂₀ or C₁-C₅ C(O)-alkyl, and R₁₃ is R₃₀; or
R₁₂ and R₁₃ are both H;
R₁₂ and R₁₃ are each independently H or substituted or unsubstituted C₁-C₅ alkyl;
R₁₂ and C3 are joined to form ring A and R₁₃ is R₃₀; or
R₁₂ and R₁₃ are joined to form ring B; or
R₁₂ and C1 are joined to form ring C and R₁₃ is R₃₀; or
C1 and C3 are joined to form ring D and R₁₂ and R₁₃ are each independently R₃₀; or
R₁₃ and C2 are joined to form ring E, m is 1, and R₁₂ is R₃₀; or
R₁₂ and R₁₃ are joined to form ring B and C1 and C3 are joined to form ring D;
wherein
Ring A, C and E are each independently a substituted or unsubstituted single spiro or fused 3-8 membered heterocyclic ring;
Ring B is a substituted or unsubstituted single, spiro or fused 3-8 membered heterocyclic ring; and
Ring D is a substituted or unsubstituted C₃-C₈ cycloalkyl;
R₇′ is H, F, Cl, Br, I, OH, O—R₂₀, SH, R₈—OH, R₈—SH, —R₈—O—R₁₀, R₈—(C₃-C₈ cycloalkyl), R₈-(3-8 membered heterocyclic ring), CF₃, CD₃, OCD₃, CN, NO₂, —CH₂CN, —R₈CN, NH₂, NHR, N(R)₂, NH(R₁₀), N(R₁₀)(R₁₁), R₈—N(R₁₀)(R₁₁), R₉—R₈—N(R₁₀)(R₁₁), B(OH)₂, —OC(O)CF₃, —OCH₂Ph, NHC(O)—R₁₀, NHCO—N(R₁₀)(R₁₁), COOH, —C(O)Ph, C(O)O—R₁₀, R₈—C(O)—R₁₀, C(O)H, C(O)—R₁₀, C₁-C₅ linear or branched C(O)-haloalkyl, —C(O)NH₂, C(O)NHR, C(O)N(R₁₀)(R₁₁), SO₂R, SO₂N(R₁₀)(R₁₁), CH(CF₃)(NH—R₁₀), C₁-C₅ linear or branched, substituted or unsubstituted alkyl, C₁-C₅ linear or branched, substituted or unsubstituted alkenyl, C₁-C₅ linear or branched, or C₃-C₈ cyclic haloalkyl, C₁-C₅ linear or branched, or C₃-C₈ cyclic alkoxy, optionally wherein at least one methylene group (CH₂) in the alkoxy is replaced with an oxygen atom, C₁-C₅ linear or branched thioalkoxy, C₁-C₅ linear or branched haloalkoxy, C₁-C₅ linear or branched alkoxyalkyl, substituted or unsubstituted C₃-C₈ cycloalkyl, substituted or unsubstituted 3-8 membered heterocyclic ring, substituted or unsubstituted aryl, substituted or unsubstituted benzyl;
R₂₀ is represented by the following structure: 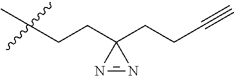 [SMILES: *CCC1(CCC#C)N=N1]
R₃₀ is H, R₂₀, F, Cl, Br, I, OH, SH, OH, alkoxy, N(R)₂, NH(R₁₀), N(R₁₀)(R₁₁), CF₃, CN, NO₂, C₁-C₅ linear or branched, substituted or unsubstituted alkyl, C₁-C₅ linear or branched alkoxy, C₁-C₅ linear or branched haloalkyl, R₈-aryl, —R₈—O—R₈—O—R₁₀, —R₈—O—R₁₀, —R₈-R₁₀, substituted or unsubstituted aryl, substituted or unsubstituted heteroaryl;
R is H, F, Cl, Br, I, OH, SH, OH, alkoxy, NH(R₁₀), NH—CH₂-cyclopropyl, N(R₁₀)(R₁₁), CF₃, CN, NO₂, COOH, C₁-C₅ linear or branched, substituted or unsubstituted alkyl, methyl, ethyl, CH₂—OH, CH₂—CH₂—OH, C₃-C₈ substituted or unsubstituted cycloalkyl, cyclopropyl, C₁-C₅ linear or branched alkoxy, isopropoxy, C₁-C₅ linear or branched haloalkyl, R₈-aryl, —R₈—O—R₈—O—R₁₀, —R₈—O—R₁₀, —R₈-R₁₀, substituted or unsubstituted aryl, substituted or unsubstituted heteroaryl;
each R₈ is independently [CH₂]ₚ
wherein p is between 1 and 10;
R₉ is [CH]_{q}, [C]_{q}
wherein q is between 2 and 10;
R₁₀ and R₁₁ are each independently H, C₁-C₅ substituted or unsubstituted linear or branched alkyl, C₁-C₅ substituted or unsubstituted linear or branched haloalky, C₁-C₅ linear or branched alkoxy, R₂₀, C(O)R, or S(O)₂R;
or R₁₀ and R₁₁ are joined to form a substituted or unsubstituted 3-8 membered heterocyclic ring,
n is an integer between 0 and 4;
or its pharmaceutically acceptable salt, stereoisomer, tautomer, hydrate, N-oxide, reverse amide analog, prodrug, isotopic variant, PROTAC, pharmaceutical product or any combination thereof.

13. The compound of claim 12, wherein R₁₀₀ is a methyl; R₅ is H; R₆ is substituted or unsubstituted 3 to 8 membered single, fused or spiro heterocyclic ring, R₈-(substituted or unsubstituted 3 to 8 membered single, fused or spiro heterocyclic ring), substituted or unsubstituted C₃-C₈ cycloalkyl, R₈-(substituted or unsubstituted C₃-C₈ cycloalkyl), or R₈—N(R₁₀)(R₁₁); R₇′ is H or F; or any combination thereof.

14. The compound of claim 12, represented by the structure of any one of the following compounds:

| Compound | |
|---|---|
| No. | Compound Name |
| 125 | N-(3-(diethylamino)propyl)-2-(4-(methylcarbamoyl)phenyl)benzo[d]imidazo[2,1- |
| | b]thiazole-7-carboxamide |
| 166 | N-(3-(diethylamino)propyl)-2-(2-fluoro-4- |
| | (methylcarbamoyl)phenyl)benzo[d]imidazo[2,1-b]thiazole-7-carboxamide |
| 174 | 2-(4-(methylcarbamoyl)phenyl)-N-(3-(piperidin-1-yl)propyl)benzo[d]imidazo[2,1- |
| | b]thiazole-7-carboxamide |
| 175 | 2-(4-(methylcarbamoyl)phenyl)-N-(3-(piperazin-1-yl)propyl)benzo[d]imidazo[2,1- |
| | b]thiazole-7-carboxamide |
| 176 | 2-(4-(methylcarbamoyl)phenyl)-N-((1-methylpyrrolidin-3- |
| | yl)methyl)benzo[d]imidazo[2,1-b]thiazole-7-carboxamide |
| 180 | N-(3-(diethylamino)propyl)-N-methyl-2-(4- |
| | (methylcarbamoyl)phenyl)benzo[d]imidazo[2,1-b]thiazole-7-carboxamide |
| 182 | (S)-N-((1,4-dioxan-2-yl)methyl)-2-(4- |
| | (methylcarbamoyl)phenyl)benzo[d]imidazo[2,1-b]thiazole-7-carboxamide |
| 183 | N-(2-(2-oxa-6-azaspiro[3.3]heptan-6-yl)ethyl)-2-(4- |
| | (methylcarbamoyl)phenyl)benzo[d]imidazo[2,1-b]thiazole-7-carboxamide |
| 184 | 2-(4-(methylcarbamoyl)phenyl)-N-(2-(4-methylpiperazin-1- |
| | yl)ethyl)benzo[d]imidazo[2,1-b]thiazole-7-carboxamide |
| 185 | 2-(4-(methylcarbamoyl)phenyl)-N-(piperidin-4-yl)benzo[d]imidazo[2,1-b]thiazole- |
| | 7-carboxamide |
| 186 | (S)-N-(1-methoxypropan-2-yl)-2-(4- |
| | (methylcarbamoyl)phenyl)benzo[d]imidazo[2,1-b]thiazole-7-carboxamide |
| 187 | N-(4-hydroxybutan-2-yl)-2-(4-(methylcarbamoyl)phenyl)benzo[d]imidazo[2,1- |
| | b]thiazole-7-carboxamide |
| 188 | (S)-N-(1-hydroxybutan-2-yl)-2-(4-(methylcarbamoyl)phenyl)benzo[d]imidazo[2,1- |
| | b]thiazole-7-carboxamide |
| 189 | N-(3-methoxypropyl)-2-(4-(methylcarbamoyl)phenyl)benzo[d]imidazo[2,1- |
| | b]thiazole-7-carboxamide |
| 190 | N-(1-(cyclopropanecarbonyl)piperidin-4-yl)-2-(4- |
| | (methylcarbamoyl)phenyl)benzo[d]imidazo[2,1-b]thiazole-7-carboxamide |
| 191 | 2-(4-(methylcarbamoyl)phenyl)-N-(3-oxo-3-(piperidin-1- |
| | yl)propyl)benzo[d]imidazo[2,1-b]thiazole-7-carboxamide |
| 192 | (R)-N-(1-hydroxy-4-methylpentan-2-yl)-2-(4- |
| | (methylcarbamoyl)phenyl)benzo[d]imidazo[2,1-b]thiazole-7-carboxamide |
| 195 | N-((3-hydroxyoxetan-3-yl)methyl)-2-(4- |
| | (methylcarbamoyl)phenyl)benzo[d]imidazo[2,1-b]thiazole-7-carboxamide |
| 196 | N-(((3R,4R)-3-hydroxypiperidin-4-yl)methyl)-2-(4- |
| | (methylcarbamoyl)phenyl)benzo[d]imidazo[2,1-b]thiazole-7-carboxamide |
| 197 | N-(1-(dimethylamino)-1-oxopropan-2-yl)-2-(4- |
| | (methylcarbamoyl)phenyl)benzo[d]imidazo[2,1-b]thiazole-7-carboxamide |
| 198 | N-(1-methylazetidin-3-yl)-2-(4-(methylcarbamoyl)phenyl)benzo[d]imidazo[2,1- |
| | b]thiazole-7-carboxamide |
| 199 | N-(1-(aminomethyl)cyclobutyl)-2-(4- |
| | (methylcarbamoyl)phenyl)benzo[d]imidazo[2,1-b]thiazole-7-carboxamide |
| 200 | (S)-N-(3-aminobutyl)-2-(4-(methylcarbamoyl)phenyl)benzo[d]imidazo[2,1- |
| | b]thiazole-7-carboxamide |
| 201 | N-(2-methoxyethyl)-2-(4-(methylcarbamoyl)phenyl)benzo[d]imidazo[2,1- |
| | b]thiazole-7-carboxamide |
| 202 | N-(2-(1-methyl-1H-pyrazol-4-yl)ethyl)-2-(4- |
| | (methylcarbamoyl)phenyl)benzo[d]imidazo[2,1-b]thiazole-7-carboxamide |
| 203 | N-(2-methoxypropyl)-2-(4-(methylcarbamoyl)phenyl)benzo[d]imidazo[2,1- |
| | b]thiazole-7-carboxamide |
| 204 | 2-(4-(methylcarbamoyl)phenyl)-N-((tetrahydrofuran-2- |
| | yl)methyl)benzo[d]imidazo[2,1-b]thiazole-7-carboxamide |
| 205 | N-(2-aminocyclohexyl)-2-(4-(methylcarbamoyl)phenyl)benzo[d]imidazo[2,1- |
| | b]thiazole-7-carboxamide |
| 206 | 2-(4-(methylcarbamoyl)phenyl)-N-(3-(trifluoromethyl)oxetan-3- |
| | yl)benzo[d]imidazo[2,1-b]thiazole-7-carboxamide |
| 207 | (S)-2-(4-(methylcarbamoyl)phenyl)-N-(1-methylpiperidin-3- |
| | yl)benzo[d]imidazo[2,1-b]thiazole-7-carboxamide |
| 208 | N-(2-(dimethylamino)butyl)-2-(4-(methylcarbamoyl)phenyl)benzo[d]imidazo[2,1- |
| | b]thiazole-7-carboxamide |
| 209 | (S)-2-(4-(methylcarbamoyl)phenyl)-N-(1-(tetrahydro-2H-pyran-4-yl)pyrrolidin-3- |
| | yl)benzo[d]imidazo[2,1-b]thiazole-7-carboxamide |
| 210 | 2-(4-(methylcarbamoyl)phenyl)-N-((3-methyltetrahydrofuran-3- |
| | yl)methyl)benzo[d]imidazo[2,1-b]thiazole-7-carboxamide |
| 211 | N-(2-isopropoxyethyl)-2-(4-(methylcarbamoyl)phenyl)benzo[d]imidazo[2,1- |
| | b]thiazole-7-carboxamide |
| 212 | (R)-2-(4-(methylcarbamoyl)phenyl)-N-((1-methylpiperidin-3- |
| | yl)methyl)benzo[d]imidazo[2,1-b]thiazole-7-carboxamide |
| 213 | (R)-N-(2-hydroxy-1-phenylethyl)-2-(4- |
| | (methylcarbamoyl)phenyl)benzo[d]imidazo[2,1-b]thiazole-7-carboxamide |
| 214 | (R)-N-((1-ethylpyrrolidin-2-yl)methyl)-2-(4- |
| | (methylcarbamoyl)phenyl)benzo[d]imidazo[2,1-b]thiazole-7-carboxamide |
| 215 | N-((1-ethylpiperidin-4-yl)methyl)-2-(4- |
| | (methylcarbamoyl)phenyl)benzo[d]imidazo[2,1-b]thiazole-7-carboxamide |
| 216 | N-((1-(dimethylamino)cyclohexyl)methyl)-2-(4- |
| | (methylcarbamoyl)phenyl)benzo[d]imidazo[2,1-b]thiazole-7-carboxamide |
| 217 | N-(2-(diisopropylamino)ethyl)-2-(4- |
| | (methylcarbamoyl)phenyl)benzo[d]imidazo[2,1-b]thiazole-7-carboxamide |
| 219 | N-((3S,4R)-4-hydroxytetrahydrofuran-3-yl)-2-(4- |
| | (methylcarbamoyl)phenyl)benzo[d]imidazo[2,1-b]thiazole-7-carboxamide |
| 220 | (S)-N-(1-aminopropan-2-yl)-2-(4-(methylcarbamoyl)phenyl)benzo[d]imidazo[2,1- |
| | b]thiazole-7-carboxamide |
| 221 | N-(1-(1H-pyrazol-1-yl)propan-2-yl)-2-(4- |
| | (methylcarbamoyl)phenyl)benzo[d]imidazo[2,1-b]thiazole-7-carboxamide |
| 222 | (S)-2-(4-(methylcarbamoyl)phenyl)-N-(pyrrolidin-3-yl)benzo[d]imidazo[2,1- |
| | b]thiazole-7-carboxamide |
| 223 | N-((4-cyclopropyl-4H-1,2,4-triazol-3-yl)methyl)-2-(4- |
| | (methylcarbamoyl)phenyl)benzo[d]imidazo[2,1-b]thiazole-7-carboxamide |
| 224 | (R)-2-(4-(methylcarbamoyl)phenyl)-N-(pyrrolidin-2- |
| | ylmethyl)benzo[d]imidazo[2,1-b]thiazole-7-carboxamide |
| 225 | (S)-2-(4-(methylcarbamoyl)phenyl)-N-(1-methylpyrrolidin-3- |
| | yl)benzo[d]imidazo[2,1-b]thiazole-7-carboxamide |
| 226 | N-((3-hydroxycyclobutyl)methyl)-2-(4- |
| | (methylcarbamoyl)phenyl)benzo[d]imidazo[2,1-b]thiazole-7-carboxamide |
| 227 | (S)-2-(4-(methylcarbamoyl)phenyl)-N-(piperidin-3-yl)benzo[d]imidazo[2,1- |
| | b]thiazole-7-carboxamide |
| 228 | (S)-N-(1-methyl-2-oxoazepan-3-yl)-2-(4- |
| | (methylcarbamoyl)phenyl)benzo[d]imidazo[2,1-b]thiazole-7-carboxamide |
| 229 | N-(4-(methylamino)butyl)-2-(4-(methylcarbamoyl)phenyl)benzo[d]imidazo[2,1- |
| | b]thiazole-7-carboxamide |
| 230 | N-((1-oxa-8-azaspiro[4.5]decan-2-yl)methyl)-2-(4- |
| | (methylcarbamoyl)phenyl)benzo[d]imidazo[2,1-b]thiazole-7-carboxamide |
| 231 | (R)-2-(4-(methylcarbamoyl)phenyl)-N-(quinuclidin-3-yl)benzo[d]imidazo[2,1- |
| | b]thiazole-7-carboxamide |
| 232 | N-(3-(3,5-dimethyl-1H-pyrazol-1-yl)propyl)-2-(4- |
| | (methylcarbamoyl)phenyl)benzo[d]imidazo[2,1-b]thiazole-7-carboxamide |
| 233 | N-((1-ethylpyrrolidin-3-yl)methyl)-2-(4- |
| | (methylcarbamoyl)phenyl)benzo[d]imidazo[2,1-b]thiazole-7-carboxamide |
| 234 | 2-(4-(methylcarbamoyl)phenyl)-N-(1-(tetrahydro-2H-pyran-4- |
| | yl)ethyl)benzo[d]imidazo[2,1-b]thiazole-7-carboxamide |
| 235 | N-(3-(1H-imidazol-1-yl)propyl)-2-(4- |
| | (methylcarbamoyl)phenyl)benzo[d]imidazo[2,1-b]thiazole-7-carboxamide |
| 236 | N-(1-methyl-5-oxopyrrolidin-3-yl)-2-(4- |
| | (methylcarbamoyl)phenyl)benzo[d]imidazo[2,1-b]thiazole-7-carboxamide |
| 237 | N-(4-(hydroxymethyl)tetrahydro-2H-pyran-4-yl)-2-(4- |
| | (methylcarbamoyl)phenyl)benzo[d]imidazo[2,1-b]thiazole-7-carboxamide |
| 238 | N-((1R,4R,5S)-2-azabicyclo[2.1.1]hexan-5-yl)-2-(4- |
| | (methylcarbamoyl)phenyl)benzo[d]imidazo[2,1-b]thiazole-7-carboxamide |
| 239 | N-(2-(dimethylamino)propyl)-2-(4- |
| | (methylcarbamoyl)phenyl)benzo[d]imidazo[2,1-b]thiazole-7-carboxamide |
| 240 | N-(2-methoxycyclopropyl)-2-(4-(methylcarbamoyl)phenyl)benzo[d]imidazo[2,1- |
| | b]thiazole-7-carboxamide |
| 241 | 2-(4-(methylcarbamoyl)phenyl)-N-(2-azaspiro[3.3]heptan-6- |
| | yl)benzo[d]imidazo[2,1-b]thiazole-7-carboxamide |
| 242 | (S)-N-(1-(1-methyl-1H-pyrazol-5-yl)propyl)-2-(4- |
| | (methylcarbamoyl)phenyl)benzo[d]imidazo[2,1-b]thiazole-7-carboxamide |
| 243 | N-(2-(2-ethyl-1H-imidazol-1-yl)ethyl)-2-(4- |
| | (methylcarbamoyl)phenyl)benzo[d]imidazo[2,1-b]thiazole-7-carboxamide |
| 244 | N-(2-methyl-2-morpholinopropyl)-2-(4- |
| | (methylcarbamoyl)phenyl)benzo[d]imidazo[2,1-b]thiazole-7-carboxamide |
| 245 | N-((1s,3s)-3-methoxycyclobutyl)-2-(4- |
| | (methylcarbamoyl)phenyl)benzo[d]imidazo[2,1-b]thiazole-7-carboxamide |
| 246 | N-((1s,3s)-3-(methylamino)cyclobutyl)-2-(4- |
| | (methylcarbamoyl)phenyl)benzo[d]imidazo[2,1-b]thiazole-7-carboxamide |
| 247 | N-((1r,3r)-3-(methylamino)cyclobutyl)-2-(4- |
| | (methylcarbamoyl)phenyl)benzo[d]imidazo[2,1-b]thiazole-7-carboxamide |
| 249 | N-(3-(5-methyl-2,5-diazabicyclo[2.2.1]heptan-2-yl)propyl)-2-(4- |
| | (methylcarbamoyl)phenyl)benzo[d]imidazo[2,1-b]thiazole-7-carboxamide |
| 250 | 4-(7-(4-(2-amino-2-oxoethyl)piperazine-1-carbonyl)benzo[d]imidazo[2,1- |
| | b]thiazol-2-yl)-N-methylbenzamide |
| 251 | N-((1-aminocyclopropyl)methyl)-2-(4- |
| | (methylcarbamoyl)phenyl)benzo[d]imidazo[2,1-b]thiazole-7-carboxamide |
| 252 | N-((1-methyl-5-oxopyrrolidin-3-yl)methyl)-2-(4- |
| | (methylcarbamoyl)phenyl)benzo[d]imidazo[2,1-b]thiazole-7-carboxamide |
| 253 | N-((1R,5S,6s)-3-azabicyclo[3.1.0]hexan-6-yl)-2-(4- |
| | (methylcarbamoyl)phenyl)benzo[d]imidazo[2,1-b]thiazole-7-carboxamide |
| 254 | N-((1-methyl-5-oxopyrrolidin-2-yl)methyl)-2-(4- |
| | (methylcarbamoyl)phenyl)benzo[d]imidazo[2,1-b]thiazole-7-carboxamide |
| 255 | 2-(4-(methylcarbamoyl)phenyl)-N-(2-oxo-2-((2,2,2- |
| | trifluoroethyl)amino)ethyl)benzo[d]imidazo[2,1-b]thiazole-7-carboxamide |
| 256 | (R)-N-(2-hydroxy-2-phenylethyl)-2-(4- |
| | (methylcarbamoyl)phenyl)benzo[d]imidazo[2,1-b]thiazole-7-carboxamide |
| 257 | 2-(4-(methylcarbamoyl)phenyl)-N-(2-(1-methylpyrrolidin-2- |
| | yl)ethyl)benzo[d]imidazo[2,1-b]thiazole-7-carboxamide |
| 258 | N-(3-hydroxy-2,2-dimethylcyclobutyl)-2-(4- |
| | (methylcarbamoyl)phenyl)benzo[d]imidazo[2,1-b]thiazole-7-carboxamide |
| 259 | N-((2,2-difluorocyclopropyl)methyl)-2-(4- |
| | (methylcarbamoyl)phenyl)benzo[d]imidazo[2,1-b]thiazole-7-carboxamide |
| 260 | N-(2-(1-hydroxycyclopentyl)ethyl)-2-(4- |
| | (methylcarbamoyl)phenyl)benzo[d]imidazo[2,1-b]thiazole-7-carboxamide |
| 261 | 2-(4-(methylcarbamoyl)phenyl)-N-((1- |
| | methylcyclopropyl)methyl)benzo[d]imidazo[2,1-b]thiazole-7-carboxamide |
| 262 | 2-(4-(methylcarbamoyl)phenyl)-N-(2-(2-methylpiperidin-1- |
| | yl)ethyl)benzo[d]imidazo[2,1-b]thiazole-7-carboxamide |
| 263 | N-((1S,2R)-2-hydroxy-2,3-dihydro-1H-inden-1-yl)-2-(4- |
| | (methylcarbamoyl)phenyl)benzo[d]imidazo[2,1-b]thiazole-7-carboxamide |
| 264 | 2-(4-(methylcarbamoyl)phenyl)-N-(1-propylpiperidin-4-yl)benzo[d]imidazo[2,1- |
| | b]thiazole-7-carboxamide |
| 269 | N-(3-(dimethylamino)cyclobutyl)-2-(4- |
| | (methylcarbamoyl)phenyl)benzo[d]imidazo[2,1-b]thiazole-7-carboxamide |
| 270 | N-(3-aminocyclohexyl)-2-(4-(methylcarbamoyl)phenyl)benzo[d]imidazo[2,1- |
| | b]thiazole-7-carboxamide |
| 271 | N-(3-(2-(hydroxymethyl)pyrrolidin-1-yl)propyl)-2-(4- |
| | (methylcarbamoyl)phenyl)benzo[d]imidazo[2,1-b]thiazole-7-carboxamide |
| 272 | N-((2-azaspiro[3.3]heptan-6-yl)methyl)-2-(4- |
| | (methylcarbamoyl)phenyl)benzo[d]imidazo[2,1-b]thiazole-7-carboxamide |
| 273 | N-(((1r,4r)-4-hydroxycyclohexyl)methyl)-2-(4- |
| | (methylcarbamoyl)phenyl)benzo[d]imidazo[2,1-b]thiazole-7-carboxamide |
| 274 | (R)-N-(1-cyclopropylethyl)-2-(4-(methylcarbamoyl)phenyl)benzo[d]imidazo[2,1- |
| | b]thiazole-7-carboxamide |
| 275 | N-benzyl-2-(4-(methylcarbamoyl)phenyl)benzo[d]imidazo[2,1-b]thiazole-7- |
| | carboxamide |
| 278 | N-((3-methylazetidin-3-yl)methyl)-2-(4- |
| | (methylcarbamoyl)phenyl)benzo[d]imidazo[2,1-b]thiazole-7-carboxamide |
| 279 | N-((5-azaspiro[2.4]heptan-6-yl)methyl)-2-(4- |
| | (methylcarbamoyl)phenyl)benzo[d]imidazo[2,1-b]thiazole-7-carboxamide |
| 280 | 2-(4-(methylcarbamoyl)phenyl)-N-(3-methylcyclobutyl)benzo[d]imidazo[2,1- |
| | b]thiazole-7-carboxamide |
| 281 | 2-(2-fluoro-4-(methylcarbamoyl)phenyl)-N-(3-(piperidin-1- |
| | yl)propyl)benzo[d]imidazo[2,1-b]thiazole-7-carboxamide |
| 282 | N-(3-(diethylamino)propyl)-2-(4-(ethylcarbamoyl)phenyl)benzo[d]imidazo[2,1- |
| | b]thiazole-7-carboxamide |
| 283 | N-(3-(diethylamino)propyl)-2-(4- |
| | (isopropylcarbamoyl)phenyl)benzo[d]imidazo[2,1-b]thiazole-7-carboxamide |
| 284 | N-(3-(diethylamino)propyl)-2-(4-((2- |
| | methoxyethyl)carbamoyl)phenyl)benzo[d]imidazo[2,1-b]thiazole-7-carboxamide |
| 285 | N-(3-(diethylamino)propyl)-2-(4-((2- |
| | hydroxyethyl)carbamoyl)phenyl)benzo[d]imidazo[2,1-b]thiazole-7-carboxamide |
| 286 | N-(3-(diethylamino)propyl)-2-(4-((1-methylpyrrolidin-3- |
| | yl)carbamoyl)phenyl)benzo[d]imidazo[2,1-b]thiazole-7-carboxamide |
| 287 | N-(3-(diethylamino)propyl)-2-(4-(piperidin-4- |
| | ylcarbamoyl)phenyl)benzo[d]imidazo[2,1-b]thiazole-7-carboxamide |
| 291 | N-(3-(diethylamino)propyl)-2-(3-fluoro-4- |
| | (methylcarbamoyl)phenyl)benzo[d]imidazo[2,1-b]thiazole-7-carboxamide |
| 295 | N-(3-(diethylamino)propyl)-2-(4-((2- |
| | (methylamino)ethyl)carbamoyl)phenyl)benzo[d]imidazo[2,1-b]thiazole-7- |
| | carboxamide |
| 296 | N-(3-(diethylamino)propyl)-2-(4-(pyrrolidin-3- |
| | ylcarbamoyl)phenyl)benzo[d]imidazo[2,1-b]thiazole-7-carboxamide |
| 297 | 2-(4-((2-aminoethyl)carbamoyl)phenyl)-N-(3- |
| | (diethylamino)propyl)benzo[d]imidazo[2,1-b]thiazole-7-carboxamide |
| 301 | (R)-2-(4-(methylcarbamoyl)phenyl)-N-(1-methylpiperidin-3- |
| | yl)benzo[d]imidazo[2,1-b]thiazole-7-carboxamide |
| 302 | (R)-2-(4-(methylcarbamoyl)phenyl)-N-(2-(1-methylpyrrolidin-2- |
| | yl)ethyl)benzo[d]imidazo[2,1-b]thiazole-7-carboxamide |
| 303 | (S)-2-(4-(methylcarbamoyl)phenyl)-N-(2-(1-methylpyrrolidin-2- |
| | yl)ethyl)benzo[d]imidazo[2,1-b]thiazole-7-carboxamide |
| 304 | N-(3-(diethylamino)propyl)-2-(4- |
| | (methylcarbamoyl)phenyl)imidazo[2′,1′:2,3]thiazolo[5,4-b]pyridine-7- |
| | carboxamide |
| 305 | 2-(4-(methylcarbamoyl)phenyl)-N-(3-(piperidin-1- |
| | yl)propyl)imidazo[2′,1′:2,3]thiazolo[5,4-b]pyridine-7-carboxamide |
| 309 | N-(3-(4-fluoropiperidin-1-yl)propyl)-2-(4- |
| | (methylcarbamoyl)phenyl)benzo[d]imidazo[2,1-b]thiazole-7-carboxamide |
| 310 | 2-(4-(methylcarbamoyl)phenyl)-N-(3-(pyrrolidin-1- |
| | yl)propyl)benzo[d]imidazo[2,1-b]thiazole-7-carboxamide |
| 311 | N-(2-(4-(methylcarbamoyl)phenyl)benzo[d]imidazo[2,1-b]thiazol-7-yl)piperidine- |
| | 4-carboxamide |
| 312 | N-methyl-4-(7-(4-(piperidin-1-yl)butanamido)benzo[d]imidazo[2,1-b[thiazol-2- |
| | yl)benzamide |
| 316 | N-(3-(diethylamino)propyl)-2-(4- |
| | (methylcarbamoyl)phenyl)imidazo[2′,1′:2,3]thiazolo[4,5-b]pyridine-7- |
| | carboxamide |
| 317 | 2-(4-(methylcarbamoyl)phenyl)-N-(3-(piperidin-1- |
| | yl)propyl)imidazo[2′,1′:2,3]thiazolo[4,5-b]pyridine-7-carboxamide |
| 319 | 2-(4-(methylcarbamoyl)phenyl)-N-(3-(piperidin-1- |
| | yl)propyl)benzo[4,5]thiazolo[3,2-b][1,2,4]triazole-6-carboxamide |
| 320 | 2-(2-fluoro-4-(methylcarbamoyl)phenyl)-N-(3-(4-fluoropiperidin-1- |
| | yl)propyl)benzo[d]imidazo[2,1-b]thiazole-7-carboxamide |
| 321 | 2-(2-fluoro-4-(methylcarbamoyl)phenyl)-N-(3-(pyrrolidin-1- |
| | yl)propyl)benzo[d]imidazo[2,1-b]thiazole-7-carboxamide |
| 322 | 2-(4-((2-(3-(but-3-yn-1-yl)-3H-diazirin-3-yl)ethyl)carbamoyl)phenyl)-N-(3- |
| | (diethylamino)propyl)benzo[d]imidazo[2,1-b]thiazole-7-carboxamide |
| 323 | N-(3-((2-(3-(but-3-yn-1-yl)-3H-diazirin-3-yl)ethyl)(ethyl)amino)propyl)-2-(4- |
| | (methylcarbamoyl)phenyl)benzo[d]imidazo[2,1-b]thiazole-7-carboxamide |
| 328 | N-(3-(diethylamino)propyl)-2-(4- |
| | (methylcarbamoyl)phenyl)benzo[4,5]thiazolo[3,2-b][1,2,4]triazole-6-carboxamide |
| 329 | N-methyl-2-(4-(methylcarbamoyl)phenyl)-N-(3-(piperidin-1- |
| | yl)propyl)benzo[d]imidazo[2,1-b]thiazole-7-carboxamide |
| 333 | N-(3-(diethylamino)propyl)-2-(2-fluoro-4-(methylcarbamoyl)phenyl)-N- |
| | methylbenzo[d]imidazo[2,1-b]thiazole-7-carboxamide |
| 349 | N-(3-(diethylamino)propyl)-2-(2-fluoro-4- |
| | (methylcarbamoyl)phenyl)benzo[4,5]thiazolo[3,2-b][1,2,4]triazole-6-carboxamide |
| 350 | N-(3-(diethylamino)propyl)-2-(4- |
| | (methylcarbamoyl)phenyl)benzo[4,5]imidazo[2,1-b]thiazole-7-carboxamide |
| 352 | N-(3-(diethylamino)propyl)-2-(2-fluoro-4- |
| | (methylcarbamoyl)phenyl)benzo[4,5]imidazo[2,1-b]thiazole-7-carboxamide |
| 353 | 2-(4-(methylcarbamoyl)phenyl)-N-(3-(piperidin-1- |
| | yl)propyl)benzo[4,5]imidazo[2,1-b]thiazole-7-carboxamide |
| 355 | N-(3-(diethylamino)propyl)-2-(2-fluoro-4- |
| | (methylcarbamoyl)phenyl)imidazo[2′,1′:2,3]thiazolo[4,5-b]pyridine-7- |
| | carboxamide |
| 357 | N-(3-(diethylamino)propyl)-2-(2-fluoro-4- |
| | (methylcarbamoyl)phenyl)imidazo[2′,1′:2,3]thiazolo[5,4-b]pyridine-7- |
| | carboxamide |
| 358 | N-(3-(diethylamino)propyl)-7-(4- |
| | (methylcarbamoyl)phenyl)imidazo[2′,1′:2,3]thiazolo[5,4-d]pyrimidine-2- |
| | carboxamide |
| 360 | N-(3-(diethylamino)propyl)-7-(2-fluoro-4- |
| | (methylcarbamoyl)phenyl)imidazo[2′,1′:2,3]thiazolo[5,4-d]pyrimidine-2- |
| | carboxamide |
| 361 | 7-(4-(methylcarbamoyl)phenyl)-N-(3-(piperidin-1- |
| | yl)propyl)imidazo[2′,1′:2,3]thiazolo[5,4-d]pyrimidine-2-carboxamide |
| 362 | N-(3-(ethylamino)propyl)-2-(2-fluoro-4- |
| | (methylcarbamoyl)phenyl)benzo[d]imidazo[2,1-b]thiazole-7-carboxamide |
| 363 | 2-(2-fluoro-4-(methylcarbamoyl)phenyl)-N-(piperidin-4-yl)benzo[d]imidazo[2,1- |
| | b]thiazole-7-carboxamide |
| 364 | N-(2-(2-fluoro-4-(methylcarbamoyl)phenyl)benzo[d]imidazo[2,1-b]thiazol-7- |
| | yl)piperidine-4-carboxamide |
| 365 | 4-(7-(4-(diethylamino)butanamido)benzo[d]imidazo[2,1-b]thiazol-2-yl)-3-fluoro- |
| | N-methylbenzamide |
| 386 | 2-(2-fluoro-4-(methylcarbamoyl)phenyl)-N-(3-(piperidin-1- |
| | yl)propyl)benzo[4,5]thiazolo[3,2-b][1,2,4]triazole-6-carboxamide |
| 389 | 2-(2-fluoro-4-(methylcarbamoyl)phenyl)-N-(3-(4-fluoropiperidin-1- |
| | yl)propyl)benzo[4,5]thiazolo[3,2-b][1,2,4]triazole-6-carboxamide |
| 391 | 2-(2-fluoro-4-(methylcarbamoyl)phenyl)-N-(3-(pyrrolidin-1- |
| | yl)propyl)benzo[4,5]thiazolo[3,2-b][1,2,4]triazole-6-carboxamide |
| 400 | N-(3-(diethylamino)propyl)-2-(4- |
| | (methylcarbamoyl)phenyl)imidazo[2′,1′:2,3]thiazolo[4,5-c]pyridine-7- |
| | carboxamide |
| 403 | N-(3-(diethylamino)propyl)-2-(2-fluoro-4- |
| | (methylcarbamoyl)phenyl)imidazo[2′,1′:2,3]thiazolo[4,5-c]pyridine-7- |
| | carboxamide |
| 406 | 2-(4-(methylcarbamoyl)phenyl)-N-(3-(piperidin-1- |
| | yl)propyl)imidazo[2′,1′:2,3]thiazolo[4,5-c]pyridine-7-carboxamide |
| 409 | 2-(2-fluoro-4-(methylcarbamoyl)phenyl)-N-(3-(piperidin-1- |
| | yl)propyl)imidazo[2′,1′:2,3]thiazolo[4,5-c]pyridine-7-carboxamide |

15. A compound represented by the structure ofany one of the following compounds:

| Compound | |
|---|---|
| No. | Compound Name |
| 101 | 2-phenyl-N-(3-(piperidin-1-yl)propyl)benzo[d]imidazo[2,1-b]thiazole-7- |
| | carboxamide |
| 103 | N-(3-(azepan-1-yl)propyl)-2-phenylbenzo[d]imidazo[2,1-b]thiazole-7-carboxamide |
| 107 | N-(3-(diethylamino)propyl)-2-phenylbenzo[d]imidazo[2,1-b]thiazole-7-carboxamide |
| 108 | N-propyl-2-(p-tolyl)benzo[d]imidazo[2,1-b]thiazole-7-carboxamide |
| 109 | N-ethyl-2-(p-tolyl)benzo[d]imidazo[2,1-b]thiazole-7-carboxamide |
| 111 | 2-(4-chlorophenyl)-N-(3-(diethylamino)propyl)benzo[d]imidazo[2,1-b]thiazole-7- |
| | carboxamide |
| 118 | N-(3-(diethylamino)propyl)-2-(m-tolyl)benzo[d]imidazo[2,1-b]thiazole-7- |
| | carboxamide |
| 123 | N-(3-(diethylamino)propyl)-2-(o-tolyl)benzo[d]imidazo[2,1-b]thiazole-7- |
| | carboxamide |
| 124 | 2-(2-chlorophenyl)-N-(3-(diethylamino)propyl)benzo[d]imidazo[2,1-b]thiazole-7- |
| | carboxamide |
| 125 | N-(3-(diethylamino)propyl)-2-(4-(methylcarbamoyl)phenyl)benzo[d]imidazo[2,1- |
| | b]thiazole-7-carboxamide |
| 126 | N-(3-(diethylamino)propyl)-2-(4-ethylphenyl)benzo[d]imidazo[2,1-b]thiazole-7- |
| | carboxamide |
| 129 | N-(3-(diethylamino)propyl)-2-(2-fluorophenyl)benzo[d]imidazo[2,1-b]thiazole-7- |
| | carboxamide |
| 130 | N-(3-(diethylamino)propyl)-2-(3-fluorophenyl)benzo[d]imidazo[2,1-b]thiazole-7- |
| | carboxamide |
| 131 | 2-(3-chlorophenyl)-N-(3-(diethylamino)propyl)benzo[d]imidazo[2,1-b]thiazole-7- |
| | carboxamide |
| 132 | 2-(4-chlorophenyl)-N-(3-(piperidin-1-yl)propyl)benzo[d]imidazo[2,1-b]thiazole-7- |
| | carboxamide |
| 133 | N-(3-(4,4-difluoropiperidin-1-yl)propyl)-2-(m-tolyl)benzo[d]imidazo[2,1-b]thiazole- |
| | 7-carboxamide |
| 134 | N-(3-morpholinopropyl)-2-(m-tolyl)benzo[d]imidazo[2,1-b]thiazole-7-carboxamide |
| 136 | N-(3-(diethylamino)propyl)-2-(4-isopropylphenyl)benzo[d]imidazo[2,1-b]thiazole-7- |
| | carboxamide formate |
| 137 | N-(3-(4-fluoropiperidin-1-yl)propyl)-2-(m-tolyl)benzo[d]imidazo[2,1-b]thiazole-7- |
| | carboxamide |
| 142 | N-(3-(diethylamino)propyl)-2-(4-methoxyphenyl)benzo[d]imidazo[2,1-b]thiazole-7- |
| | carboxamide |
| 143 | N-(3-(diethylamino)propyl)-2-(2-fluoro-3-methylphenyl)benzo[d]imidazo[2,1- |
| | b]thiazole-7-carboxamide |
| 144 | N-(3-(diethylamino)propyl)-2-(2-fluoro-5-methylphenyl)benzo[d]imidazo[2,1- |
| | b]thiazole-7-carboxamide |
| 145 | 2-(3-cyanophenyl)-N-(3-(diethylamino)propyl)benzo[d]imidazo[2,1-b]thiazole-7- |
| | carboxamide |
| 150 | N-(3-(2-oxopyrrolidin-1-yl)propyl)-2-(m-tolyl)benzo[d]imidazo[2,1-b]thiazole-7- |
| | carboxamide |
| 152 | N-(3-(diethylamino)propyl)-2-(3-methoxyphenyl)benzo[d]imidazo[2,1-b]thiazole-7- |
| | carboxamide |
| 154 | N-(3-(diethylamino)propyl)-2-(4-(dimethylcarbamoyl)phenyl)benzo[d]imidazo[2,1- |
| | b]thiazole-7-carboxamide formate |
| 155 | N-(3-(ethylamino)propyl)-2-(m-tolyl)benzo[d]imidazo[2,1-b]thiazole-7-carboxamide |
| 160 | 2-(4-cyanophenyl)-N-(3-(diethylamino)propyl)benzo[d]imidazo[2,1-b]thiazole-7- |
| | carboxamide |
| 164 | N-(3-(diethylamino)propyl)-2-(4-(methylamino)phenyl)benzo[d]imidazo[2,1- |
| | b]thiazole-7-carboxamide |
| 167 | N-(3-(diethylamino)propyl)-2-(3-(methylcarbamoyl)phenyl)benzo[d]imidazo[2,1- |
| | b]thiazole-7-carboxamide |
| 169 | N-(3-(diethylamino)propyl)-2-(3-isopropylphenyl)benzo[d]imidazo[2,1-b]thiazole-7- |
| | carboxamide |
| 172 | 4-(7-((3-(diethylamino)propyl)carbamoyl)benzo[d]imidazo[2,1-b]thiazol-2- |
| | yl)benzoic acid |
| 177 | N-(3-(ethyl(2,2,2-trifluoroethyl)amino)propyl)-2-(m-tolyl)benzo[d]imidazo[2,1- |
| | b]thiazole-7-carboxamide |
| 178 | 2-(m-tolyl)-N-(3-((2,2,2-trifluoroethyl)amino)propyl)benzo[d]imidazo[2,1- |
| | b]thiazole-7-carboxamide |
| 181 | N-(3-(diethylamino)propyl)-N-methyl-2-(m-tolyl)benzo[d]imidazo[2,1-b]thiazole-7- |
| | carboxamide |
| 193 | N-(3-(ethyl(2,2,2-trifluoroethyl)amino)propyl)-N-methyl-2-(m- |
| | tolyl)benzo[d]imidazo[2,1-b]thiazole-7-carboxamide |
| 384 | 2-(4-(aminomethyl)-3-chloro-5-fluorophenyl)-N-(3-(piperidin-1- |
| | yl)propyl)benzo[d]imidazo[2,1-b]thiazole-7-carboxamide |

16. The compound according to claim 1, wherein the compound is a c-MYC mRNA translation modulator, a c-MYC mRNA transcription regulator, a c-MYC inhibitor or any combination thereof.

17. A pharmaceutical composition comprising the compound of claim 1 and a pharmaceutically acceptable carrier.

18. A method of treating, suppressing, reducing the severity, reducing the risk of developing or inhibiting cancer in a subject, comprising administering a compound according to claim 1, to a subject suffering from cancer under conditions effective to treat, suppress, reduce the severity, reduce the risk of developing, or inhibit cancer in said subject.

19. The method of claim 18,
wherein the cancer is selected from the list of: breast cancer, ovarian carcinoma, acute myeloid leukemia, chronic myelogenous leukemia, Hodgkin's and Burkitt's lymphoma, diffuse large Bcell lymphoma, prostate cancer, colon cancer, gastric cancer, primary central nervous system lymphoma, glioblastoma, medulloblastoma, melanoma, non-small cell lung carcinoma, germinal center-derived lymphomas, esophageal squamous cell carcinoma, osteosarcoma, bladder cancer, pancreatic cancer, lung adenocarcinoma, BRAF V600E thyroid cancer, choroid plexus carcinoma, colitis-associated cancer, epithelial ovarian cancer, colorectal cancer, pancreatic cancer and uterine cancer;
wherein the cancer is early cancer, advanced cancer, invasive cancer, metastatic cancer, drug resistant cancer or any combination thereof;
wherein the subject has been previously treated with chemotherapy, immunotherapy, radiotherapy, biological therapy, surgical intervention, or any combination thereof;
wherein the compound is administered in combination with an anti-cancer therapy;
or any combination thereof.

20. The method of claim 19, wherein the anti-cancer therapy is chemotherapy, immunotherapy, radiotherapy, biological therapy, surgical intervention, or any combination thereof.

21. A method for suppressing, reducing or inhibiting tumor growth in a subject, comprising administering a compound according to claim 1, to a subject under conditions effective to suppress, reduce or inhibit tumor growth in said subject.

22. A method of modulating c-MYC mRNA translation, or regulating c-MYC mRNA transcription in a cell, comprising contacting a compound according to claim 1 with a cell, thereby modulating c-MYC mRNA translation, or regulating c-MYC mRNA transcription in said cell.

23. The method of claim 22, wherein said method is carried out
(a) by regulating c-MYC mRNA splicing (inclusion or exclusion of untranslated region or alternative usage of exons);
(b) by regulation of c-MYC mRNA modifications;
(c) by regulation of the interaction of RNA binding protein with c-MYC mRNA thereby changing mRNA localization;
(d) by regulating c-MYC mRNA localization in the cytoplasm;
(e) by regulating ribosomes or ribosome accessory factor to c-MYC mRNA;
(f) by reducing the amount of c-MYC protein in the cell;
or any combination thereof.
